SEQUENCE LISTING (64 sequences)

SEQ ID NO: 1 - protein, 16 aa, synthetic construct
  REGION 1..16: note = source = /note="Description of Artificial Sequence: Synthetic peptide"
RSSQSIVHSVGNTFLE

SEQ ID NO: 2 - protein, 7 aa, synthetic construct
  REGION 1..7: note = source = /note="Description of Artificial Sequence: Synthetic peptide"
KVSNRFS

SEQ ID NO: 3 - protein, 9 aa, synthetic construct
  REGION 1..9: note = source = /note="Description of Artificial Sequence: Synthetic peptide"
FQGSQFPYT

SEQ ID NO: 4 - protein, 10 aa, synthetic construct
  REGION 1..10: note = source = /note="Description of Artificial Sequence: Synthetic peptide"
GYEFSRSWMN

SEQ ID NO: 5 - protein, 18 aa, synthetic construct
  REGION 1..18: note = source = /note="Description of Artificial Sequence: Synthetic peptide"
GRIYPGDGDTNYSGKFKG

SEQ ID NO: 6 - protein, 11 aa, synthetic construct
  REGION 1..11: note = source = /note="Description of Artificial Sequence: Synthetic peptide"
DGSSWDWYFDV

SEQ ID NO: 7 - protein, 107 aa, synthetic construct
  REGION 1..107: note = source = /note="Description of Artificial Sequence: Synthetic polypeptide"
DIQMTQSPSSLSASVGDRVTITCSASQGISNYLNWYQQKPGKTVKLLIYYTSNLHSGVPSRFSGSGSGTDYTLTISSLQPEDFATYYCQQYSELPWTFGQGTKVEIK

SEQ ID NO: 8 - protein, 120 aa, synthetic construct
  REGION 1..120: note = source = /note="Description of Artificial Sequence: Synthetic polypeptide"
EVQLVESGPALVKPTQTLTLTCTVSGFSLTGYSVNWIRQPPGKALEWLGMIWGDGSTDYNSALKSRLTISKDTSKNQVVLTMTNMDPVDTATYYCARDYYFNYASWFAYWGQGTLVTVSS

SEQ ID NO: 9 - protein, 11 aa, synthetic construct
  REGION 1..11: note = source = /note="Description of Artificial Sequence: Synthetic peptide"
SASQGISNYLN

SEQ ID NO: 10 - protein, 7 aa, synthetic construct
  REGION 1..7: note = source = /note="Description of Artificial Sequence: Synthetic peptide"
YTSNLHS

SEQ ID NO: 11 - protein, 9 aa, synthetic construct
  REGION 1..9: note = source = /note="Description of Artificial Sequence: Synthetic peptide"
QQYSELPWT

SEQ ID NO: 12 - protein, 10 aa, synthetic construct
  REGION 1..10: note = source = /note="Description of Artificial Sequence: Synthetic peptide"
GFSLTGYSVN

SEQ ID NO: 13 - protein, 16 aa, synthetic construct
  REGION 1..16: note = source = /note="Description of Artificial Sequence: Synthetic peptide"
MIWGDGSTDYNSALKS

SEQ ID NO: 14 - protein, 12 aa, synthetic construct
  REGION 1..12: note = source = /note="Description of Artificial Sequence: Synthetic peptide"
DYYVNYASWFAY

SEQ ID NO: 15 - protein, 214 aa, synthetic construct
  REGION 1..214: note = source = /note="Description of Artificial Sequence: Synthetic polypeptide"
DIQMTQSPSSLSASVGDRVTITCSASQGISNYLNWYQQKPGKTVKLLIYYTSNLHSGVPSRFSGSGSGTDYTLTISSLQPEDFATYYCQQYSELPWTFGQGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 16 - protein, 450 aa, synthetic construct
  REGION 1..450: note = source = /note="Description of Artificial Sequence: Synthetic polypeptide"
EVQLVESGPALVKPTQTLTLTCTVSGFSLTGYSVNWIRQPPGKALEWLGMIWGDGSTDYNSALKSRLTISKDTSKNQVVLTMTNMDPVDTATYYCARDYYFNYASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 112 aa, synthetic construct
  REGION 1..112: note = source = /note="Description of Artificial Sequence: Synthetic polypeptide"
DIVMTQSPDSLAVSLGERATINCRASQSVSGSRFTYMHWYQQKPGQPPKLLIKYASILESGVPDRFSGSGSGTDFTLTISSLQAEDVAVYYCQHSWEIPPWTFGQGTKVEIK

SEQ ID NO: 18 - protein, 118 aa, synthetic construct
  REGION 1..118: note = source = /note="Description of Artificial Sequence: Synthetic polypeptide"
EVQLVQSGAEVKKPGASVKVSCKASGYSFTGYWMNWVRQAPGQGLEWIGMIHPLDAEIRANQKFRDRVTITVDTSTSTAYLELSSLRSEDTAVYYCARGTYDGGFEYWGQGTLVTVSS

SEQ ID NO: 19 - protein, 15 aa, synthetic construct
  REGION 1..15: note = source = /note="Description of Artificial Sequence: Synthetic peptide"
RASQSVSGSRFTYMH

SEQ ID NO: 20 - protein, 7 aa, synthetic construct
  REGION 1..7: note = source = /note="Description of Artificial Sequence: Synthetic peptide"
YASILES

SEQ ID NO: 21 - protein, 10 aa, synthetic construct
  REGION 1..10: note = source = /note="Description of Artificial Sequence: Synthetic peptide"
QHSWEIPPWT

SEQ ID NO: 22 - protein, 5 aa, synthetic construct
  REGION 1..5: note = source = /note="Description of Artificial Sequence: Synthetic peptide"
GYWMN

SEQ ID NO: 23 - protein, 17 aa, synthetic construct
  REGION 1..17: note = source = /note="Description of Artificial Sequence: Synthetic peptide"
MIHPLDAEIRANQKFRD

SEQ ID NO: 24 - protein, 9 aa, synthetic construct
  REGION 1..9: note = source = /note="Description of Artificial Sequence: Synthetic peptide"
GTYDGGFEY

SEQ ID NO: 25 - protein, 219 aa, synthetic construct
  REGION 1..219: note = source = /note="Description of Artificial Sequence: Synthetic polypeptide"
DIVMTQSPDSLAVSLGERATINCRASQSVSGSRFTYMHWYQQKPGQPPKLLIKYASILESGVPDRFSGSGSGTDFTLTISSLQAEDVAVYYCQHSWEIPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 448 aa, synthetic construct
  REGION 1..448: note = source = /note="Description of Artificial Sequence: Synthetic polypeptide"
EVQLVQSGAEVKKPGASVKVSCKASGYSFTGYWMNWVRQAPGQGLEWIGMIHPLDAEIRANQKFRDRVTITVDTSTSTAYLELSSLRSEDTAVYYCARGTYDGGFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 17 aa, synthetic construct
  REGION 1..17: note = source = /note="Description of Artificial Sequence: Synthetic peptide"
MIHPMDSEIRANQKFRD

SEQ ID NO: 28 - protein, 17 aa, synthetic construct
  REGION 1..17: note = source = /note="Description of Artificial Sequence: Synthetic peptide"
MIHPLDSEIRANQKFRD

SEQ ID NO: 29 - protein, 9 aa, synthetic construct
  REGION 1..9: note = source = /note="Description of Artificial Sequence: Synthetic peptide"
GTYDGGFKY

SEQ ID NO: 30 - protein, 219 aa, synthetic construct
  REGION 1..219: note = source = /note="Description of Artificial Sequence: Synthetic polypeptide"
DIVMTQSPDSLAVSLGERATINCRASQSVSGSRFTYMHWYQQKPGQPPKLLIKYASILESGVPDRFSGSGSGTDFTLTISSLQAEDVAVYYCQHSWEIPPWTFGQGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 31 - protein, 448 aa, synthetic construct
  REGION 1..448: note = source = /note="Description of Artificial Sequence: Synthetic polypeptide"
EVQLVQSGAEVKKPGASVKVSCKASGYSFTGYWMNWVRQAPGQGLEWIGMIHPLDAEIRANQKFRDRVTITVDTSTSTAYLELSSLRSEDTAVYYCARGTYDGGFEYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 11 aa, synthetic construct
  REGION 1..11: note = source = /note="Description of Artificial Sequence: Synthetic peptide"
KASDLIHNWLA

SEQ ID NO: 33 - protein, 8 aa, synthetic construct
  REGION 1..8: note = source = /note="Description of Artificial Sequence: Synthetic peptide"
YGATSLET

SEQ ID NO: 34 - protein, 9 aa, synthetic construct
  REGION 1..9: note = source = /note="Description of Artificial Sequence: Synthetic peptide"
QQYWTTPFT

SEQ ID NO: 35 - protein, 11 aa, synthetic construct
  REGION 1..11: note = source = /note="Description of Artificial Sequence: Synthetic peptide"
GYSITNDYAWN

SEQ ID NO: 36 - protein, 17 aa, synthetic construct
  REGION 1..17: note = source = /note="Description of Artificial Sequence: Synthetic peptide"
GYISYSGYTTYNPSLKS

SEQ ID NO: 37 - protein, 9 aa, synthetic construct
  REGION 1..9: note = source = /note="Description of Artificial Sequence: Synthetic peptide"
ARWASGLDY

SEQ ID NO: 38 - protein, 108 aa, synthetic construct
  REGION 1..108: note = source = /note="Description of Artificial Sequence: Synthetic polypeptide"
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIKR

SEQ ID NO: 39 - protein, 116 aa, synthetic construct
  REGION 1..116: note = source = /note="Description of Artificial Sequence: Synthetic polypeptide"
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWASGLDYWGQGTLVTVSS

SEQ ID NO: 40 - protein, 11 aa, synthetic construct
  REGION 1..11: note = source = /note="Description of Artificial Sequence: Synthetic peptide"
GYSITSDYAWN

SEQ ID NO: 41 - protein, 17 aa, synthetic construct
  REGION 1..17: note = source = /note="Description of Artificial Sequence: Synthetic peptide"
GYISNSGSTSYNPSLKS

SEQ ID NO: 42 - protein, 15 aa, synthetic construct
  REGION 1..15: note = source = /note="Description of Artificial Sequence: Synthetic peptide"
ERNYDYDDYYYAMDY

SEQ ID NO: 43 - protein, 17 aa, synthetic construct
  REGION 1..17: note = source = /note="Description of Artificial Sequence: Synthetic peptide"
KSSQSLLYRSNQKNYLA

SEQ ID NO: 44 - protein, 7 aa, synthetic construct
  REGION 1..7: note = source = /note="Description of Artificial Sequence: Synthetic peptide"
WASTRES

SEQ ID NO: 45 - protein, 9 aa, synthetic construct
  REGION 1..9: note = source = /note="Description of Artificial Sequence: Synthetic peptide"
QQYYNYPRT

SEQ ID NO: 46 - protein, 124 aa, synthetic construct
  REGION 1..124: note = source = /note="Description of Artificial Sequence: Synthetic polypeptide"
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSS

SEQ ID NO: 47 - protein, 113 aa, synthetic construct
  REGION 1..113: note = source = /note="Description of Artificial Sequence: Synthetic polypeptide"
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIK

SEQ ID NO: 48 - protein, 10 aa, synthetic construct
  REGION 1..10: note = source = /note="Description of Artificial Sequence: Synthetic peptide"
GFSFSDFAMS

SEQ ID NO: 49 - protein, 18 aa, synthetic construct
  REGION 1..18: note = source = /note="Description of Artificial Sequence: Synthetic peptide"
ATIGRVAFHTYYPDSMKG

SEQ ID NO: 50 - protein, 13 aa, synthetic construct
  REGION 1..13: note = source = /note="Description of Artificial Sequence: Synthetic peptide"
ARHRGFDVGHFDF

SEQ ID NO: 51 - protein, 16 aa, synthetic construct
  REGION 1..16: note = source = /note="Description of Artificial Sequence: Synthetic peptide"
RSSETLVHSSGNTYLE

SEQ ID NO: 52 - protein, 7 aa, synthetic construct
  REGION 1..7: note = source = /note="Description of Artificial Sequence: Synthetic peptide"
RVSNRFS

SEQ ID NO: 53 - protein, 9 aa, synthetic construct
  REGION 1..9: note = source = /note="Description of Artificial Sequence: Synthetic peptide"
FQGSFNPLT

SEQ ID NO: 54 - protein, 120 aa, synthetic construct
  REGION 1..120: note = source = /note="Description of Artificial Sequence: Synthetic polypeptide"
EVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSS

SEQ ID NO: 55 - protein, 113 aa, synthetic construct
  REGION 1..113: note = source = /note="Description of Artificial Sequence: Synthetic polypeptide"
DIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIKR

SEQ ID NO: 56 - protein, 117 aa, synthetic construct [sequence omitted]

SEQ ID NO: 57 - protein, 112 aa, synthetic construct
  REGION 1..112: note = source = /note="Description of Artificial Sequence: Synthetic polypeptide"
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKR

SEQ ID NO: 58 - protein, 10 aa, synthetic construct
  REGION 1..10: note = source = /note="Description of Artificial Sequence: Synthetic peptide"
GYTFSSYWIE

SEQ ID NO: 59 - protein, 18 aa, synthetic construct
  REGION 1..18: note = source = /note="Description of Artificial Sequence: Synthetic peptide"
GEILPGGGDTNYNEIFKG

SEQ ID NO: 60 - protein, 10 aa, synthetic construct
  REGION 1..10: note = source = /note="Description of Artificial Sequence: Synthetic peptide"
TRRVPIRLDY

SEQ ID NO: 61 - protein, 15 aa, synthetic construct
  REGION 1..15: note = source = /note="Description of Artificial Sequence: Synthetic peptide"
KASQSVDYEGDSFLN

SEQ ID NO: 62 - protein, 7 aa, synthetic construct
  REGION 1..7: note = source = /note="Description of Artificial Sequence: Synthetic peptide"
AASNLES

SEQ ID NO: 63 - protein, 9 aa, synthetic construct
  REGION 1..9: note = source = /note="Description of Artificial Sequence: Synthetic peptide"
QQSNEDPLT

SEQ ID NO: 64 - protein, 1255 aa, Homo sapiens [sequence omitted]

(19) United States
(12) Patent Application Publication
(10) Pub. No.: US 2024/0002379 A1
(43) Pub. Date: Jan. 4, 2024

(54) ASYMMETRIC CONJUGATE COMPOUNDS

(71) Applicant: Pheon Therapeutics Ltd., Hertfordshire (GB)

(72) Inventors: Paul Joseph Mark Jackson, Hertfordshire (GB); David Thurston, Hertfordshire (GB); Khondaker Mirazur Rahman, Hertfordshire (GB)

(21) Appl. No.: 18/066,137

(22) Filed: Dec. 14, 2022

Related U.S. Application Data
(63) Continuation of application No. 16/189,310, filed on Nov. 13, 2018, now abandoned.
Application No. 16/189,310 is a continuation-in-part of application No. PCT/GB2017/051331, filed on May 12, 2017.

(30) Foreign Application Priority Data
May 13, 2016 (GB) 1608408.9
Dec. 1, 2016 (GB) 1620407.5

(51) Int. Cl. C07D 471/04; A61K 47/68; A61P 35/00

(52) U.S. Cl. CPC C07D 471/04; A61K 47/6803; A61P 35/00

(57) ABSTRACT

The invention relates to compound of formula (I):

A-X₁-L-X₂-B (I)

and salts, solvates and tautomers thereof, which are useful as medicaments, in particular as anti-proliferative agents and for use as a drug in an antibody-drug conjugate; wherein A is a group selected from:

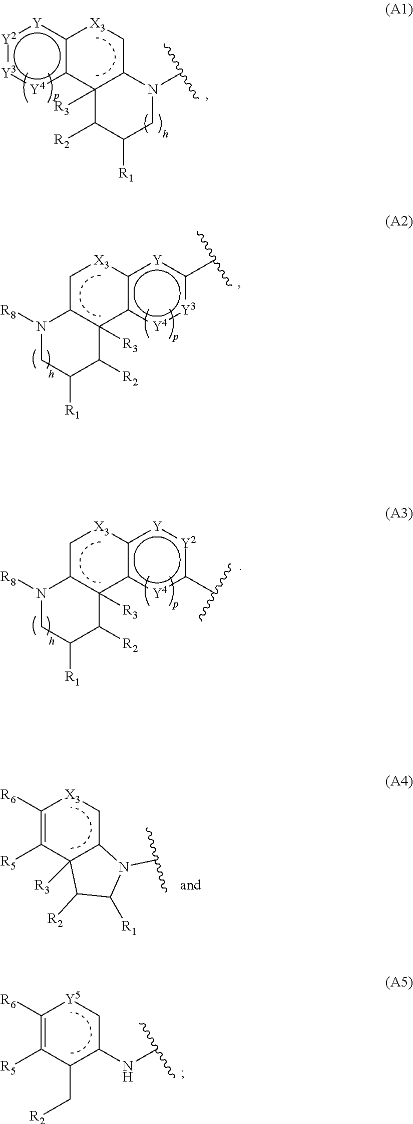

X₁ and X₂ are independently selected from O, S, NR₂₈, CR₂₈R₂₉, CR₂₈R₂₉O, C(═O), C(═O)NR₂₈, NR₂₈C(═O), C(O)—R^{A}—C(O)—NH, C(O)—R^{A}—NH—C(O), C(O)—NH—R^{A}—C(O), NH—C(O)—R^{A}—C(O), NH—C(O)—R^{A}—C(O)—NH, NH—C(O)—R^{A}—NH—C(O), C(O)—NH—R^{A}—NH—C(O), C(O)—NH—R^{A}—C(O)—NH, O—C(O) and C(O)—O or is absent;
L is selected from an amino acid, a peptide chain having from 2 to 12 amino acids, a paraformaldehyde chain —(OCH₂)₁₋₂₄—, a polyethylene glycol chain —(OCH₂CH₂)₁₋₁₂— and —(CH₂)ₘ—Y⁶—(CH₂)ₙ— wherein Y⁶ is selected from —(CH₂)_{z}— and a group (L1) a group (L1) that is selected from arylene, monocyclic heteroarylene, monocyclic cycloalkylene, monocyclic cycloalkenylene and monocyclic heterocyclylene groups optionally substituted with up to three optional substituent groups; and
B is a polycyclic group selected from:

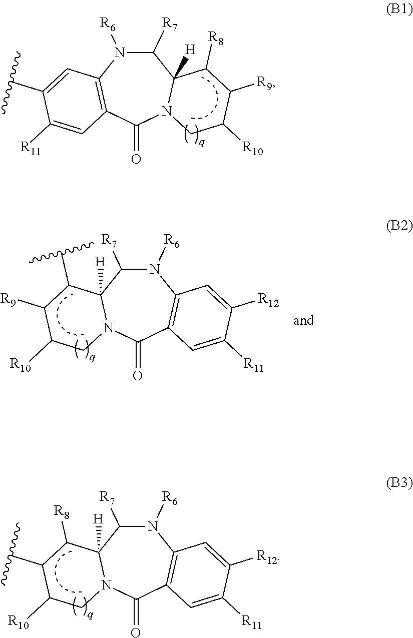

### CROSS-REFERENCE TO RELATED APPLICATIONS

This application is a continuation of U.S. patent application Ser. No. 16/189,310, filed Nov. 13, 2018, which is a continuation-in-part of International Patent Application number PCT/GB2017/051331, filed May 12, 2017 (currently published). International Patent Application number PCT/GB2017/051331 cites the priority of Great Britain Patent Application number GB1608408.9, filed May 13, 2016 (currently abandoned), and Great Britain Patent Application number GB1620407.5, filed Dec. 1, 2016 (currently abandoned).

### SEQUENCE LISTING

The instant application contains a Sequence Listing which has been submitted electronically in .XML format and is hereby incorporated by reference in its entirety.

Said .XML copy, created on Aug. 16, 2023, is named “133186-5009-US01_ASYMMETRIC_CONJUGATE_COMPOUNDS” and is 67,256 Bytes in size.

### FIELD OF THE INVENTION

The invention relates to asymmetric conjugate compounds comprising a guanine-alkylating moiety [e.g., pyrrolobenzodiazepine (PBD) or a Pyrridinobenzodiazepines (PDD)] linked to an adenine-alkylating moiety [e.g., a cyclopropylpyrolo[e]ndolone (CPI) or cyclopropyl[c]benzo[e]indolone (CBI)], and to salts, solvates and tautomers thereof, which are useful as medicaments, in particular as anti-proliferative agents.

### BACKGROUND TO THE INVENTION

Pyrridinobenzodiazepines (PDDs) and pyrrolobenzodiazepines (PBDs) are sequence-selective DNA minor-groove binding agents. The PBDs were originally discovered in Streptomyces species (1-5). They are tricyclic in nature, and are comprised of fused 6-7-5-membered rings that comprise an anthranilate (A ring), a diazepine (B ring) and a pyrrolidine (C ring) (3). The related PDDs are comprised of fused 6-7-6-membered rings. PBDs are characterized by an electrophilic N₁₀=C₁₁ imine group (as shown below) or the hydrated equivalent, a carbinolamine [NH—CH(OH)], or a carbinolamine alkyl ether ([NH—CH(OR, where R=alkyl)] which can form a covalent bond to a C₂-amino group of guanine in DNA to form a DNA adduct (6).

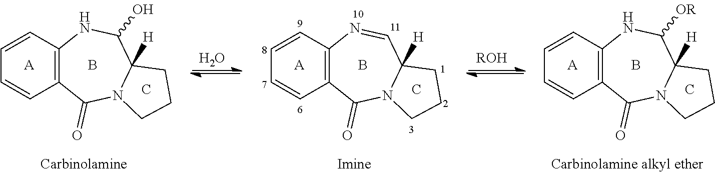

The natural products interact in the minor groove of the DNA helix with excellent fit (i.e., good “isohelicity”) due to a right-handed longitudinal twist induced by a chiral C₁₁ₐ-position which has the (S)-configuration (6). The DNA adduct has been reported to inhibit a number of biological processes including the binding of transcription factors (7-9) and the function of enzymes such as endonucleases (10, 11) and RNA polymerase (12). PBD monomers (e.g., anthramycin) have been shown by footprinting (6), NMR (13, 14), molecular modeling (15) and X-ray crystallography (16) to span three base pairs and to have a thermodynamic preference for the sequence 5′-Pu-G-Pu-3′ (where Pu=purine, and G is the reacting guanine) (17) and a kinetic preference for 5′-Py-G-Py-3′ (where Py=Pyrimidine).

PBDs are thought to interact with DNA by first locating at a low-energy binding sequence (i.e., a 5′-Pu-G-Pu-3′ triplet) through Van der Waals, hydrogen bonding and electrostatic interactions (7). Then, once in place, a nucleophilic attack by the exocyclic C₂-amino group of the central guanine occurs to form the covalent adduct (7). Once bound, the PBD remains anchored in the DNA minor groove, avoiding DNA repair by causing negligible distortion of the DNA helix (16). The ability of PBDs to form an adduct in the minor groove and cross-link DNA enables them to interfere with DNA processing and, hence, their potential for use as antiproliferative agents. PDDs are also minor groove-binding molecules with similar mechanism of action and cytotoxicity.

WO 2010/091150 discloses a dimer of a 6-7-6 ring system linked via their A-rings. WO 2015/028850 discloses 6-7-5 ring system PBD dimers that are linked via phosphine oxide containing linkers attached to their aromatic A-rings. In addition, WO 2015/028850 discloses a dimer compound containing a 6-7-6 ring system linked via the key phosphine oxide containing linkers. Such PBD dimers can form sequence selective G-G cross-links in the DNA minor groove (18).

Bizelesin and related dimeric CPI molecules have been investigated as stand-alone anticancer agents but they were abandoned as potential clinical agents due to significant liver toxicity (19). Such dimeric CPI molecules are capable of binding to adenine bases (A) and so forming sequence selective A-A cross-links in the DNA minor groove.

More recently PBD and CPI units have been joined together to create asymmetric molecules capable of forming cross-links to both G and A bases, the first example was UTA-6026 (20).

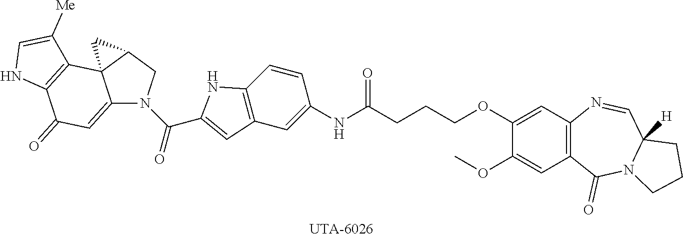

The most persuasive evidence for significant interstrand cross-linking ability and cytotoxicity of asymmetric molecules of this type relate to 27eS (21) which was significantly more cytotoxic than most PBD dimers.

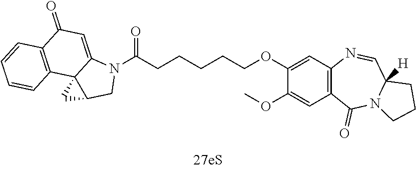 [SMILES: COc1cc2c(cc1OCCCCCC(=O)N1C[C@@H]3C[C@]34C1=CC(=O)c1ccccc14)N=C[C@@H]1CCCN1C2=O]

A related asymmetric molecule, shown below Compound 11, has also been disclosed but this has significantly lower cross-linking efficiency than 27eS or UTA-6026 (22).

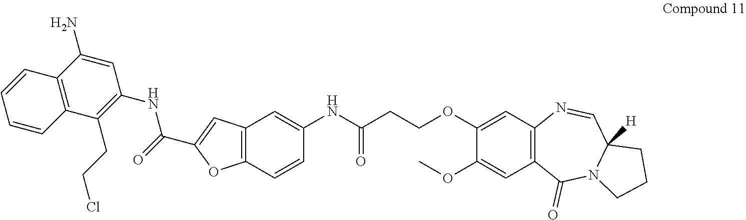 [SMILES: COc1cc2c(cc1OCCC(=O)Nc1ccc3oc(C(=O)Nc4cc(N)c5ccccc5c4CCCl)cc3c1)N=C[C@@H]1CCCN1C2=O]

WO2015023355 discloses drug moieties comprising CBI dimers and also drug moieties comprising a CBI linked to an unsubstituted PBD. WO2015023355 also discloses antibody-drug conjugates comprising such drug moieties; furthermore, immunoconjugates comprising such drug moieties linked to antibodies that bind HER2 are disclosed in WO2016040723.

No agents that act through cross-linking A to G base pairs have been developed for clinical use.

A number of clinically-used cancer therapeutics (e.g., cisplatin) work by forming intra- and/or interstrand covalent DNA cross-links. Although the molecular steps that lead from cross-linking to cell death, and the reasons for tumour cell selectivity, are not fully understood, potency and selectivity are known to relate to DNA repair deficiencies in tumour cells. All clinically-used agents of this type form inter- or intrastrand cross-links between guanine (G) bases. Cells do not usually encounter bis-links between guanine (G) and adenine (A) base pairs, and agents forming these lesions have not been developed for clinical use. Thus, there exists a need for further asymmetric compounds and related derivatives that are therapeutically active for treating a variety of different diseases (in particular, proliferative diseases) as G-A lesions should be more difficult to repair, leading to enhanced lethality.

The present application reports asymmetric conjugate compounds comprising a PBD/PDD and a CPI/CBI. The inventors have discovered asymmetric conjugate compounds providing properties, such as improved cross-linking efficiency, cytoxicity and modified sequence-selectivity that results in effective compounds. Furthermore, extensive rational design based on proprietary molecular modelling techniques has suggested that modification of the central linker between the alkylating moieties may further enhance DNA-binding and cytotoxicity.

The present invention seeks to overcome problem(s) associated with the prior art.

### SUMMARY OF THE INVENTION

The present invention provides a compound of formula (I):

A-X₁-L-X₂-B (I)

and salts, solvates and tautomers thereof, for use as a drug in an antibody-drug conjugate,
wherein;
A is a group selected from:

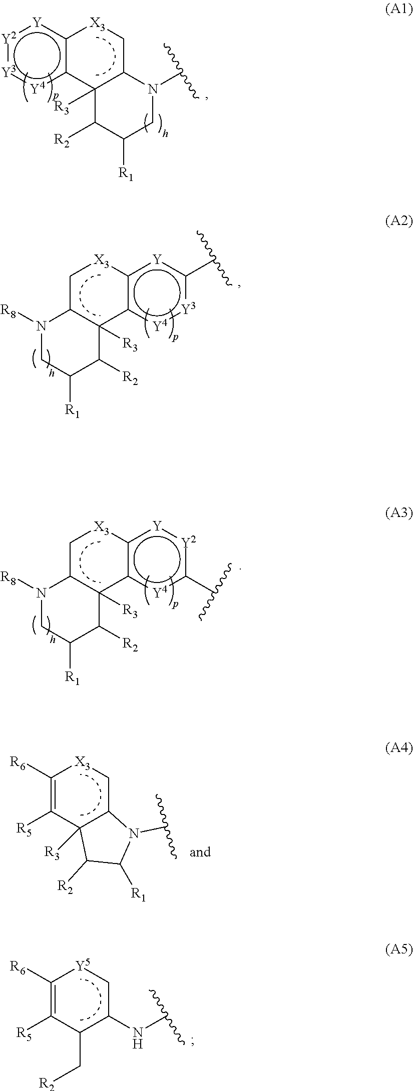

h is 0 or 1;
R₁ is selected from H and halogen;
either R₂ is selected from —CH₂-halogen, C₁₋₆ alkyl and H, and R₃ is H; or R₂ and R₃ together with the carbon atoms to which they are attached form a cyclopropyl ring;
p is 0 or 1; and when p is 1 then Y is C—R₇, Y² is C—R₆, Y3 is C—R₅ and Y4 is C—R₄; and for (A1) and (A2) when p is 0 either (a) Y is selected from N—R₁₉, O and S; Y² is selected from C—R₆ and N; and Y³ is C—R₅; or (b) Y³ is selected from N—R₁₉, O and S; Y² is selected from C—R₆ and N; and Y is C—R₇; and for (A3) when p is 0, Y is selected from N—R₁₉, O and S; and Y² is selected from C—R₆ and N;
R₄, R₅, R₆ and R₇ are each independently selected from H and R₂₀, or one of R₄ and R₅, or R₅ and R₆, or R₆ and R₇ together with the carbon atoms to which they are attached form a 6-membered aryl, or a 5- or 6-membered cyclic, heterocyclic, or heteroaryl ring optionally substituted with up to three independently selected optional R₂₀ groups;
R₈ is selected from selected from H, nitrogen protecting groups and R₂₀;
X₃ is selected from C═O, C—OH and C—R′″; or Y⁵ is selected from C═O, C—OH, C—NH₂ and C—R′″; with the carbon forming part of the ring; and
   when X₃ or Y⁵ is C═O then [symbol] represents an α,β-unsaturated double bond conjugated with the C═O; and when X₃ is C—OH or C—R′″ or Y⁵ is C—OH, C—NH₂ or C—R′″ then [symbol] represents the double bonds of an aromatic 6-membered ring and R₃ is absent;
wherein R′″ is a prodrug moiety containing carbonyl, carbamoyl, glycosyl, O-amino, O-acylamino, para-aminobenzyl ether, peptidyl or phosphate groups;
X₁ is selected from O, S, NR₂₁, CR₂₁R₂₂, CR₂₁R₂₂O, C(═O), C(═O)NR₂₁, NR₂₁C(═O), C(O)—R^{A}—C(O)—NH, C(O)—R^{A}—NH—C(O), C(O)—NH—R^{A}—C(O), NH—C(O)—R^{A}—C(O), NH—C(O)—R^{A}—C(O)—NH, NH—C(O)—R^{A}—NH—C(O), C(O)—NH—R^{A}—NH—C(O), C(O)—NH—R^{A}—C(O)—NH, O—C(O) and C(O)—O or is absent;
L is selected from an amino acid, a peptide chain having from 2 to 12 amino acids, a paraformaldehyde chain —(OCH₂)₁₋₂₄—, a polyethylene glycol chain —(OCH₂CH₂)₁₋₁₂— and —(CH₂)ₘ—Y⁶—(CH₂)ₙ— wherein
   m is an integer selected from 0 to 12,
   n is an integer selected from 0 to 12, and
   Y⁶ is selected from —(CH₂)_{z}— and a group (L1) that is selected from arylene, monocyclic heteroarylene, monocyclic cycloalkylene, monocyclic cycloalkenylene and monocyclic heterocyclylene groups optionally substituted with up to three independently selected optional R₂₀ groups;
   z is an integer selected from 1 to 5;
X₂ is selected from O, S, NR₂₃, CR₂₃R₂₄, CR₂₃R₂₄O, C(═O), C(═O)NR₂₃, NR₂₄C(═O), C(O)—R^{A}—C(O)—NH, C(O)—R^{A}—NH—C(O), C(O)—NH—R^{A}—C(O), NH—C(O)—R^{A}—C(O), NH—C(O)—R^{A}—C(O)—NH, NH—C(O)—R^{A}—NH—C(O), C(O)—NH—R^{A}—NH—C(O), C(O)—NH—R^{A}—C(O)—NH, O—C(O) and C(O)—O or is absent;
B is a polycyclic group selected from:

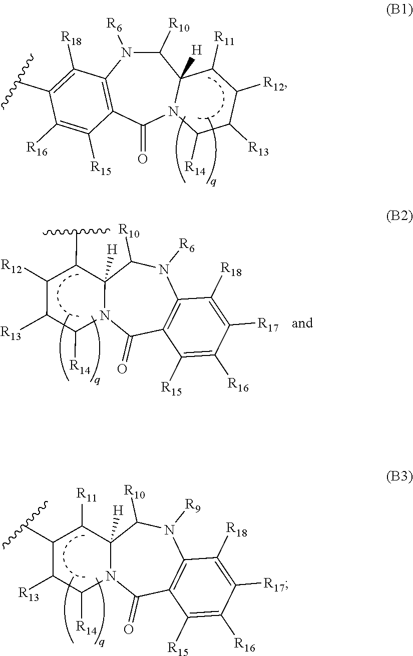

the dotted lines indicate the optional presence of one or more double bonds; q is 0 or 1;
and R₉ and R₁₀ are selected such that either:
   (i) R₉ and R₁₀ together form a double bond;
   (ii) R₉ is H and R₁₀ is OH;
   (iii) R₉ is H and R₁₀ is OC₁₋₆ alkyl;
   (iv) R₉ is selected from SO₃H, nitrogen protecting groups and R₂₀; and R₁₀ is H; or
   (v) R₉ is H or C₁₋₆ alkyl, and R₁₀ is oxo or H;
R₁₁, R₁₂, R₁₃ and R₁₄ are independently selected from H, R₂₀, R₂₅, ═CH₂, ═CH—(CH₂)ₛ—CH₃, ═CH—(CH₂)ₛ—R₂₅, ═O, (CH₂)ₛ—OR₂₅, (CH₂)ₛ—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)ₛ—SO₂R₂₅, O—SO₂R₂₅, (CH₂)ₛ—C(O)R₂₅ and (CH₂)ₛ—C(O)NR₂₅R₂₆;
   or one of R₁₁ and R₁₂, R₁₂ and R₁₃, or R₁₃ and R₁₄ together with the carbon atoms to which they are attached form a 6-membered aryl, or a 5- or 6-membered cyclic, heterocyclic, or heteroaryl ring optionally substituted with up to three independently selected optional R₂₀ groups;
each s is an integer independently selected from 0 to 6;
each t is an integer independently selected from 1 to 6;
R₁₅, R₁₆, R₁₇ and R₁₈ are independently selected from H and R₂₀;
each R₂₀ is independently selected from (CH₂)ⱼ—OH, C₁₋₆ alkyl, OC₁₋₆ alkyl, OCH₂Ph, (CH₂)ⱼ—CO₂R₂₇, O—(CH₂)ₖ—NR₂₇R₂₈, (CH₂)ⱼ—NR₂₇R₂₈, C(═O)—NH—(CH₂)ₖ—NR₂₇R₂₈, C(═O)—NH—C₆H₄—(CH₂)ⱼ—R₂₇ and C(═O)—NH—(CH₂)ₖ—C(═NH)NR₂₇R₂₈;
   each j is an integer independently selected from 0 to 6;
   each k is an integer independently selected from 1 to 6;
each R₁₉, R₂₁, R₂₂, R₂₃, R₂₄, R₂₆, R₂₇ and R₂₈ is independently selected from H and C₁₋₆ alkyl; and
each R₂₅ is independently selected from H, C₁₋₁₂ alkyl, C₅₋₉ heteroaryl, C₆₋₁₅ heteroarylalkyl, phenyl and C₇₋₁₂ aralkyl groups; wherein the heteroaryl, heteroarylalkyl, phenyl and aralkyl groups are optionally substituted with up to three independently selected optional R₂₀ groups;
   each R^{A} is independently selected from:
      —NR^{B}—T¹—NR^{C}— where R^{B} and R^{C} are each independently selected from H and C₁₋₈ alkyl, or together R^{B} and R^{c} join to form a ring and together are (CH₂)₂₋₃, where T¹ is selected from —C(O), —C(O)(CH₂)₀₋₅₀C(O)—, —C(O)PhC(O)— where Ph is 1,3- or 1,4-phenylene;
      -het- wherein het is a mono-, bi-, or tricyclic heteroarylene of 5 to 12 members, containing one, two, or three heteroatoms independently selected from O, N, S, P and B, wherein het is optionally substituted up to three independently selected optional R₂₀ groups;
      -X^{A}-T²—X^{A}—, where T² is:

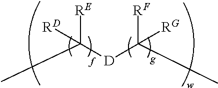

wherein each X^{A} is independently selected from a bond, —NH—, —N(C₁₋₈ alkyl)-, —O— and —S—,
each R^{D}, R^{E}, R^{F}, and R^{G} are each independently H or R₂₀, or R^{D} and R^{E} form a ring system, or R^{F} and R^{G} form a ring system, or both R^{D} and R^{E}, and R^{F} and R^{G} independently form ring systems, where said ring systems are independently selected from —C₁-C₁₀ heterocyclyl or —C₃-C₈ carbocyclycl, or R^{D}, R^{E}, R^{F}, and R^{G} are each bonds to different carbons on D, wherein f and g are each independently an integer from 0 to 50 and w is an integer from 1 to 50, and wherein D is a bond or is selected from the group consisting of —S—, —C₁-C₅ alkylene-, —C₆-C₁₄ arylene-, —C₆-C₁₄ heteroarylene-, —C₁-C₅ heteroalkylene-, —C₇-C₂₂ aralkylene, —C₁-C₁₀ heterocyclo and —C₃-C₈ carbocyclo, where said —C₁-C₅ alkylene-, —C₆-C₁₄ arylene-, —C₆-C₁₄ heteroarylene-, —C₁-C₅ heteroalkylene-, —C₇-C₂₂ aralkylene, —C₁-C₁₀ heterocyclo and —C₃-C₈ carbocyclo are optionally substituted up to three independently selected optional R₂₀ groups;
with the proviso that when the compound is:

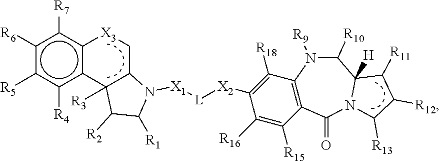

that at least one of R₁₁, R₁₂ and R₁₃ is independently selected from C₅₋₉ heteroaryl, C₆₋₁₅ heteroarylalkyl, phenyl and C₇₋₁₂ aralkyl groups and these groups are optionally substituted with up to three independently selected optional R₂₀ groups, or that one of R₁₁ and R₁₂ or R₁₂ and R₁₃, or R₁₃ together with the carbon atoms to which they are attached form a 6-membered aryl, or a 5- or 6-membered cyclic, heterocyclic, or heteroaryl ring optionally substituted with up to three independently selected optional R₂₀ groups;
with the proviso that R₅ and R₆ are each independently selected from H and R₂₀ when B, q and A are selected as (B1), 0 and (A4) respectively;
with the proviso that when R₂ is C₁₋₆ alkyl or H, that R₉ and R₁₀ are selected from options (i), (ii), (iii) or (iv); and
with the proviso that when (v) R₉ is H or C₁₋₆ alkyl, and R₁₀ is oxo or H; then either R₂ is CH halogen and R₃ is H;
   or R₂ and R₃ together with the carbon atoms to which they are attached form a cyclopropyl ring.

In a further aspect there is provided a compound of formula (I):

A-X₁-L-X₂-B (I)
and salts, solvates and tautomers thereof,
wherein;
A is a group selected from:

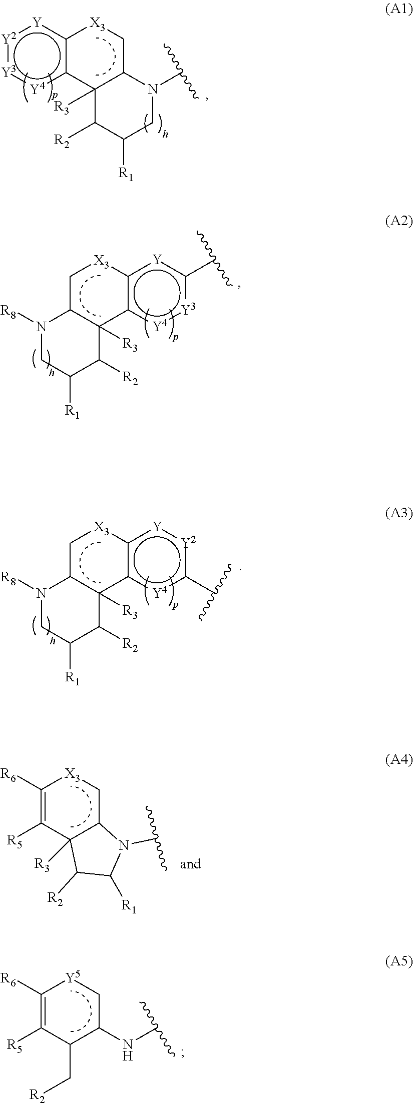

h is 0 or 1;
R₁ is selected from H and halogen;
either R₂ is selected from —CH₂-halogen, C₁₋₆ alkyl and H, and R₃ is H; or R₂ and R₃ together with the carbon atoms to which they are attached form a cyclopropyl ring;
p is 0 or 1; and when p is 1 then Y is C—R₇, Y² is C—R₆, Y³ is C—R_{S} and Y⁴ is C—R₄; and for (A1) and (A2) when p is 0 either (a) Y is selected from N—R₁₉, O and S; Y² is selected from C—R₆ and N; and Y³ is C—R₅; or (b) Y³ is selected from N—R₁₉, O and S; Y² is selected from C—R₆ and N; and Y is C—R₇; and for (A3) when p is 0, Y is selected from N—R₁₉, O and S; and Y² is selected from C—R₆ and N;
R₄, R₅, R₆ and R₇ are each independently selected from H and R₂₀, or one of R₄ and R₅, or R₅ and R₆, or R₆ and R₇ together with the carbon atoms to which they are attached form a 6-membered aryl, or a 5- or 6-membered cyclic, heterocyclic, or heteroaryl ring optionally substituted with up to three independently selected optional R₂₀ groups;
R₈ is selected from selected from H, nitrogen protecting groups and R₂₀;
X₃ is selected from C═O, C—OH and C—R′″; or Y⁵ is selected from C═O, C—OH, C—NH₂ and C—R′″; with the carbon forming part of the ring; and
   when X₃ or Y⁵ is C═O then [symbol] represents an α,β-unsaturated double bond conjugated with the C═O; and when X₃ is C—OH or C—R′″; or Y⁵ is C—OH, C—NH₂ or C—R′″ then [symbol] represents the double bonds of an aromatic 6-membered ring and R₃ is absent;
wherein R′″ is a prodrug moiety containing carbonyl, carbamoyl, glycosyl, O-amino, O-acylamino, para-aminobenzyl ether, peptidyl or phosphate groups
X₁ is selected from O, S, NR₂₁, CR₂₁R₂₂, CR₂₁R₂₂O, C(═O), C(═O)NR₂₁, NR₂₁C(═O), C(O)—R^{A}—C(O)—NH, C(O)—R^{A}—NH—C(O), C(O)—NH—R^{A}—C(O), NH—C(O)—R^{A}—C(O), NH—C(O)—R^{A}—C(O)—NH, NH—C(O)—R^{A}—NH—C(O), C(O)—NH—R^{A}—NH—C(O), C(O)—NH—R^{A}—C(O)—NH, O—C(O) and C(O)—O or is absent;
L is selected from an amino acid, a peptide chain having from 2 to 12 amino acids, a paraformaldehyde chain —(OCH₂)₁₋₂₄—, a polyethylene glycol chain —(OCH₂CH₂)₁₋₁₂— and —(CH₂)ₘ—Y⁶—(CH₂)ₙ— wherein
   m is an integer selected from 0 to 12,
   n is an integer selected from 0 to 12, and
   Y⁶ is selected from —(CH₂)_{z}— and a group (L1) that is selected from arylene, monocyclic heteroarylene, monocyclic cycloalkylene, monocyclic cycloalkenylene and monocyclic heterocyclylene groups optionally substituted with up to three independently selected optional R₂₀ groups;
   z is an integer selected from 1 to 5;
X₂ is selected from O, S, NR₂₃, CR₂₃R₂₄, CR₂₃R₂₄O, C(═O), C(═O)NR₂₃, NR₂₄C(═O), C(O)—R^{A}—C(O)—NH, C(O)—R^{A}—NH—C(O), C(O)—NH—R^{A}—C(O), NH—C(O)—R^{A}—C(O), NH—C(O)—R^{A}—C(O)—NH, NH—C(O)—R^{A}—NH—C(O), C(O)—NH—R^{A}—NH—C(O), C(O)—NH—R^{A}—C(O)—NH, O—C(O) and C(O)—O or is absent;
B is a polycyclic group selected from:

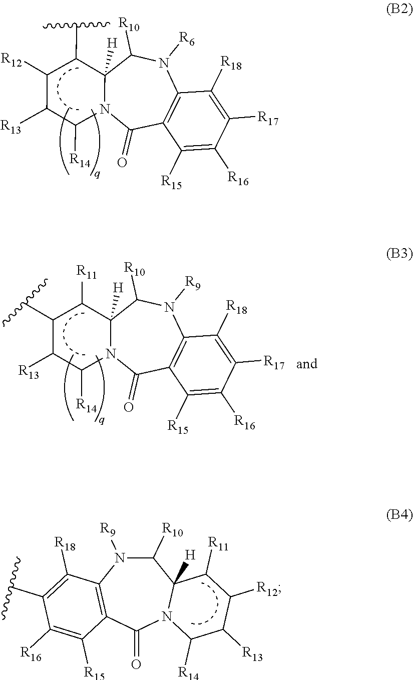

the dotted lines indicate the optional presence of one or more double bonds; q is 0 or 1;
and R₉ and R₁₀ are selected such that either:
   (i) R₉ and R₁₀ together form a double bond;
   (ii) R₉ is H and R₁₀ is OH;
   (iii) R₉ is H and R₁₀ is OC₁₋₆ alkyl;
   (iv) R₉ is selected from SO₃H, nitrogen protecting groups and R₂₀; and R₁₀ is H; or
   (v) R₉ is H or C₁₋₆ alkyl, and R₁₀ is oxo or H
R₁₁, R₁₂, R₁₃ and R₁₄ are independently selected from H, R₂₀, R₂₅, ═CH₂, ═CH—(CH₂)ₛ—CH₃, ═CH—(CH₂)ₛ—R₂₅, ═O, (CH₂)ₛ—OR₂₅, (CH₂)ₛ—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)ₛ—SO₂R₂₅, O—SO₂R₂₅, (CH₂)ₛ—C(O)R₂₅ and (CH₂)ₛ—C(O)NR₂₅R₂₆;
   or one of R₁₁ and R₁₂, R₁₂ and R₁₃, or R₁₃ and R₁₄ together with the carbon atoms to which they are attached form a 6-membered aryl, or a 5- or 6-membered cyclic, heterocyclic, or heteroaryl ring optionally substituted with up to three independently selected optional R₂₀ groups;
each s is an integer independently selected from 0 to 6;
each t is an integer independently selected from 1 to 6;
R₁₅, R₁₆, R₁₇ and R₁₈ are independently selected from H and R₂₀;
each R₂₀ is independently selected from (CH₂)ⱼ—OH, C₁₋₆ alkyl, OC₁₋₆ alkyl, OCH₂Ph, (CH₂)ⱼ—CO₂R₂₇, O—(CH₂)ₖ—NR₂₇R₂₈, (CH₂)ⱼ—NR₂₇R₂₈, C(═O)—NH—(CH₂)ₖ—NR₂₇R₂₈; C(═O)—NH—C₆H₄—(CH₂)ⱼ—R₂₇ and C(═O)—NH—(CH₂)ₖ—C(═NH)NR₂₇R₂₈;
   each j is an integer independently selected from 0 to 6;
   each k is an integer independently selected from 1 to 6;
each R₁₉, R₂₁, R₂₂, R₂₃, R₂₄, R₂₆, R₂₇ and R₂₈ is independently selected from H and C₁₋₆ alkyl; and
each R₂₅ is independently selected from H, C₁₋₁₂ alkyl, C₅₋₉ heteroaryl, C₆₋₁₅ heteroarylalkyl, phenyl and C₇₋₁₂ aralkyl groups; wherein the heteroaryl, heteroarylalkyl, phenyl and aralkyl groups are optionally substituted with up to three independently selected optional R₂₀ groups;
each R^{A} is independently selected from:
   —NR^{B}—T¹—NR^{C}— where R^{B} and R^{C} are each independently selected from H or C_{1≢}alkyl, or together R^{B} and R^{c} join to form a ring and together are (CH₂)₂₋₃, where T¹ is selected from —C(O), —C(O)(CH₂)₀₋₅₀C(O)—, —C(O)PhC(O)— where Ph is 1,3- or 1,4-phenylene;
   -het- wherein het is a mono-, bi-, or tricyclic heteroarylene of 5 to 12 members, containing one, two, or three heteroatoms independently selected from O, N, S, P and B, wherein het is optionally substituted up to three independently selected optional R₂₀ groups;
   -X^{A}-T²—X^{A}-, where T² is:

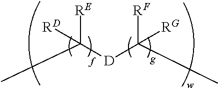

wherein each X^{A} is independently selected from a bond, —NH—, —N(C₁₋ₛ alkyl)-, —O— and —S—,
each R^{D}, R^{E}, R^{F}, and R^{G} are each independently H or R₂₀, or R^{D} and R^{E} form a ring system, or R^{F} and R^{G} form a ring system, or both R^{D} and R^{E}, and R^{F} and R^{G} independently form ring systems, where said ring systems are independently selected from —C₁-C₁₀ heterocyclyl or —C₃-C₈ carbocyclycl, or R^{D}, R^{E}, R^{F}, and R^{G} are each bonds to different carbons on D, wherein f and g are each independently an integer from 0 to 50 and w is an integer from 1 to 50, and wherein D is a bond or is selected from the group consisting of —S—, —C₁-C₅ alkylene-, —C₆-C₁₄ arylene-, —C₆-C₁₄ heteroarylene-, —C₁-C₅ heteroalkylene-, —C₇-C₂₂ aralkylene, —C₁-C₁₀ heterocyclo and —C₃-C₈ carbocyclo, where said —C₁-C₅ alkylene-, —C₆-C₁₄ arylene-, —C₆-C₁₄ heteroarylene-, —C₁-C₅ heteroalkylene-, —C₇-C₂₂ aralkylene, —C₁-C₁₀ heterocyclo and —C₃-C₈ carbocyclo are optionally substituted up to three independently selected optional R₂₀ groups;
with the proviso that when R₂ is C₁₋₆ alkyl or H, that R₉ and R₁₀ are selected from options (i), (ii), (iii) or (iv); and
with the proviso that when (v) R₉ is H or C₁₋₆ alkyl, and R₁₀ is oxo or H; then either R₂ is —CH₂-halogen and R₃ is H;
   or R₂ and R₃ together with the carbon atoms to which they are attached form a cyclopropyl ring.

In a further aspect, there is provided a compound of formula (I):

A-X₁-L-X₂-B (I)
and salts, solvates and tautomers thereof, for use as a drug in an antibody-drug conjugate,
wherein;
A is a group selected from:

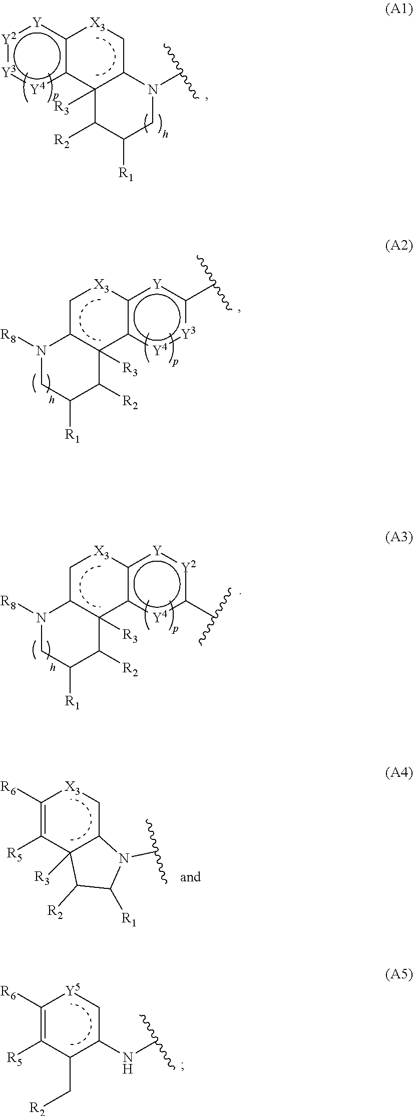

h is 0 or 1;
R₁ is selected from H and halogen;
either R₂ is selected from —CH₂-halogen and H, and R₃ is H;
   or R₂ and R₃ together with the carbon atoms to which they are attached form a cyclopropyl ring;
p is 0 or 1; and when p is 1 then Y is C—R₇, Y² is C—R₆, Y³ is C—R_{S} and Y⁴ is C—R₄; and for (A1) and (A2) when p is 0 either (a) Y is selected from N—R₁₉, O and S; Y² is selected from C—R₆ and N; and Y³ is C—R₅; or (b) Y³ is selected from N—R₁₉, O and S; Y² is selected from C—R₆ and N; and Y is C—R₇; and for (A3) when p is 0, Y is selected from N—R₁₉, O and S; and Y² is selected from C—R₆ and N;
R₄, R₅, R₆ and R₇ are each independently selected from H and R₂₀,
   or one of R₄ and R₅, or R₅ and R₆, or R₆ and R₇ together with the carbon atoms to which they are attached form a 6-membered aryl, or a 5- or 6-membered cyclic, heterocyclic, or heteroaryl ring optionally substituted with up to three independently selected optional R₂₀ groups;
R₈ is selected from H, nitrogen protecting groups and R₂₀;
X₃ is selected from C═O and C—OH, or Y⁵ is selected from C═O, C—OH and C—NH₂, with the carbon forming part of the ring; and
   when X₃ or Y⁵ is C═O then [symbol] represents an α,β-unsaturated double bond conjugated with the C═O; and when X₃ is C—OH or Y⁵ is C—OH or C—NH₂ then [symbol] represents the double bonds of an aromatic 6-membered ring and R₃ is absent;
X₁ is selected from O, S, NR₂₁, CR₂₁R₂₂, CR₂₁R₂₂O, C(═O), C(═O)NR₂₁, NR₂₁C(═O), C(O)—R^{A}—C(O)—NH, C(O)—R^{A}—NH—C(O), C(O)—NH—R^{A}—C(O), NH—C(O)—R^{A}—C(O), NH—C(O)—R^{A}—C(O)—NH, NH—C(O)—R^{A}—NH—C(O), C(O)—NH—R^{A}—NH—C(O), C(O)—NH—R^{A}—C(O)—NH, O—C(O) and C(O)—O or is absent;
L is selected from an amino acid, a peptide chain having from 2 to 12 amino acids, a paraformaldehyde chain —(OCH₂)₁₋₂₄—, a polyethylene glycol chain —(OCH₂CH₂)₁₋₁₂— and —(CH₂)ₘ—Y⁶—(CH₂)ₙ— wherein
   m is an integer selected from 0 to 12,
   n is an integer selected from 0 to 12, and
   Y⁶ is selected from —(CH₂)_{z}— and a group (L1) that is selected from arylene, monocyclic heteroarylene, monocyclic cycloalkylene, monocyclic cycloalkenylene and monocyclic heterocyclylene groups optionally substituted with up to three independently selected optional R₂₀ groups;
   z is an integer selected from 1 to 5;
X₂ is selected from O, S, NR₂₃, CR₂₃R₂₄, CR₂₃R₂₄O, C(═O), C(═O)NR₂₃, NR₂₄C(═O), C(O)—R^{A}—C(O)—NH, C(O)—R^{A}—NH—C(O), C(O)—NH—R^{A}—C(O), NH—C(O)—R^{A}—C(O), NH—C(O)—R^{A}—C(O)—NH, NH—C(O)—R^{A}—NH—C(O), C(O)—NH—R^{A}—NH—C(O), C(O)—NH—R^{A}—C(O)—NH, O—C(O) and C(O)—O or is absent;
B is a polycyclic group selected from:

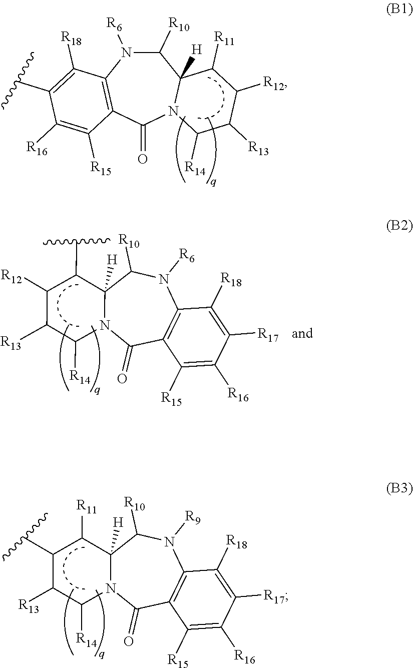

the dotted lines indicate the optional presence of one or more double bonds;
q is 0 or 1;
and either:
   (i) R₉ and R₁₀ together form a double bond;
   (ii) R₉ is H and R₁₀ is OH;
   (iii) R₉ is H and R₁₀ is OC₁₋₆ alkyl; or
   (iv) R₉ is selected from SO₃H, nitrogen protecting groups and R₂₀; and R₁₀ is H;
R₁₁, R₁₂, R₁₃ and R₁₄ are independently selected from H, R₂₀, R₂₅, ═CH₂, ═CH—(CH₂)ₛ—CH₃, ═CH—(CH₂)ₛ—R₂₅, ═O, (CH₂)ₛ—OR₂₅, (CH₂)ₛ—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)ₛ—SO₂R₂₅, O—SO₂R₂₅, (CH₂)ₛ—C(O)R₂₅ and (CH₂)ₛ—C(O)NR₂₅R₂₆;
   or one of R₁₁ and R₁₂, R₁₂ and R₁₃, or R₁₃ and R₁₄ together with the carbon atoms to which they are attached form a 6-membered aryl, or a 5- or 6-membered cyclic, heterocyclic, or heteroaryl ring optionally substituted with up to three independently selected optional R₂₀ groups;
each s is an integer independently selected from 0 to 6;
each t is an integer independently selected from 1 to 6;
R₁₅, R₁₆, R₁₇ and R₁₈ are independently selected from H and R₂₀;
each R₂₀ is independently selected from (CH₂)ⱼ—OH, C₁₋₆ alkyl, OC₁₋₆ alkyl, OCH₂Ph, (CH₂)ⱼ—CO₂R₂₇, O—(CH₂)ₖ—NR₂₇R₂₈, (CH₂)ⱼ—NR₂₇R₂₈, C(═O)—NH—(CH₂)ₖ—NR₂₇R₂₈, C(═O)—NH—C₆H₄—(CH₂)ⱼ—R₂₇ and C(═O)—NH—(CH₂)ₖ—C(═NH)NR₂₇R₂₈;
   each j is an integer independently selected from 0 to 6;
   each k is an integer independently selected from 1 to 6;
each R₁₉, R₂₁, R₂₂, R₂₃, R₂₄, R₂₆, R₂₇ and R₂₈ is independently selected from H and C₁₋₆ alkyl; and each R₂₅ is independently selected from H, C₁₋₁₂ alkyl, C₅₋₉ heteroaryl, C₆₋₁₅ heteroarylalkyl, phenyl and C₇₋₁₂ aralkyl groups; wherein the heteroaryl, heteroarylalkyl, phenyl and aralkyl groups are optionally substituted with up to three independently selected optional R₂₀ groups;
each R^{A} is independently selected from:
   —NR^{B}—T¹—NR^{C}— where R^{B} and R^{C} are each independently selected from H and C₁₋₈ alkyl, or together R^{B} and R^{c} join to form a ring and together are (CH₂)₂₋₃, where T¹ is selected from —C(O), —C(O)(CH₂)₀₋₅₀C(O)—, —C(O)PhC(O)— where Ph is 1,3- or 1,4-phenylene;
   -het- wherein het is a mono-, bi-, or tricyclic heteroarylene of 5 to 12 members, containing one, two, or three heteroatoms independently selected from O, N, S, P and B, wherein het is optionally substituted up to three independently selected optional R₂₀ groups;
   -X^{A}-T²-X^{A}-, where T² is:

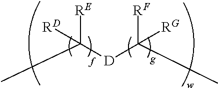

wherein each X^{A} is independently selected from a bond, —NH—, —N(C₁₋₈ alkyl)-, —O— and —S—,
each R^{D}, R^{E}, R^{F}, and R^{G} are each independently H or R₂₀, or R^{D} and R^{E} form a ring system, or R^{F} and R^{G} form a ring system, or both R^{D} and R^{E}, and R^{F} and R^{G} independently form ring systems, where said ring systems are independently selected from —C₁-C₁₀ heterocyclyl or —C₃-C₈ carbocyclycl, or R^{D}, R^{E}, R^{F}, and R^{G} are each bonds to different carbons on D, wherein f and g are each independently an integer from 0 to 50 and w is an integer from 1 to 50, and wherein D is a bond or is selected from the group consisting of —S—, —C₁-C₅ alkylene-, —C₆-C₁₄ arylene-, —C₆-C₁₄ heteroarylene-, —C₁-C₅ heteroalkylene-, —C₇-C₂₂ aralkylene, —C₁-C₁₀ heterocyclo and —C₃-C₈ carbocyclo, where said —C₁-C₅ alkylene-, —C₆-C₁₄ arylene-, —C₆-C₁₄ heteroarylene-, —C₁-C₅ heteroalkylene-, —C₇-C₂₂ aralkylene, —C₁-C₁₀ heterocyclo and —C₃-C₈ carbocyclo are optionally substituted up to three independently selected optional R₂₀ groups;
with the proviso that when the compound is:

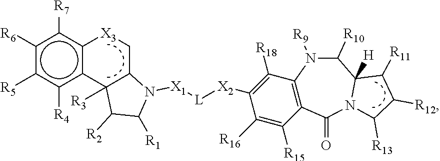

at least one of R₁₁, R₁₂ and R₁₃ is independently selected from C₅₋₉ heteroaryl, C₆₋₁₅ heteroarylalkyl, phenyl and C₇₋₁₂ aralkyl groups and these groups are optionally substituted with up to three independently selected optional R₂₀ groups, or that one of R₁₁ and R₁₂ or R₁₂ and R₁₃, or R₁₃ together with the carbon atoms to which they are attached form a 6-membered aryl, or a 5- or 6-membered cyclic, heterocyclic, or heteroaryl ring optionally substituted with up to three independently selected optional R₂₀ groups;
and with the proviso that R₅ and R₆ are each independently selected from H and R₂₀ when B, q and A are selected as (B1), 0 and (A4) respectively.

In a further aspect, there is provided a compound of formula (I):

A-X₁-L-X₂-B (I)
and salts, solvates and tautomers thereof,
wherein:
A is a group selected from:

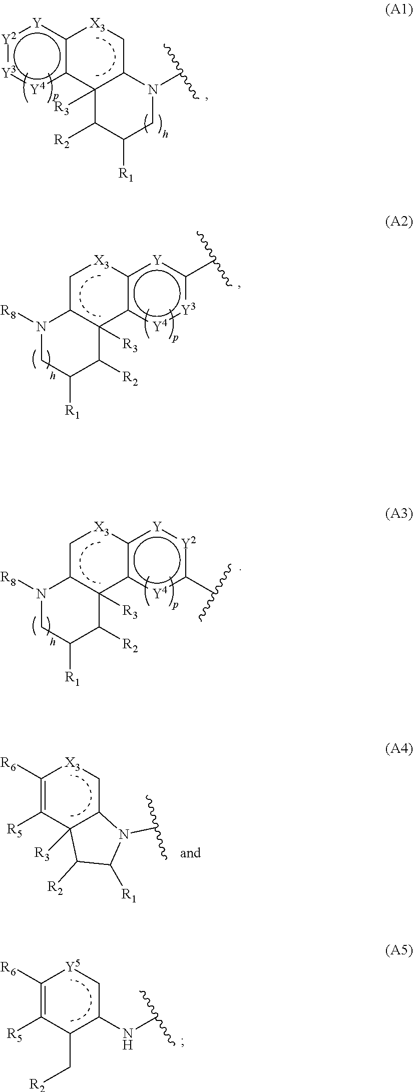

h is 0 or 1;
R₁ is selected from H and halogen;
either R₂ is selected from —CH₂-halogen and H, and R₃ is H;
   or R₂ and R₃ together with the carbon atoms to which they are attached form a cyclopropyl ring;
p is 0 or 1; and when p is 1 then Y is C—R₇, Y² is C—R₆, Y³ is C—R_{S} and Y⁴ is C—R₄; and for (A1) and (A2) when p is 0 either (a) Y is selected from N—R₁₉, O and S; Y² is selected from C—R₆ and N; and Y³ is C—R₅; or (b) Y³ is selected from N—R₁₉, O and S; Y² is selected from C—R₆ and N; and Y is C—R₇; and for (A3) when p is 0, Y is selected from N—R₁₉, O and S; and Y² is selected from C—R₆ and N;
R₄, R₅, R₆ and R₇ are each independently selected from H and R₂₀, or one of R₄ and R₅, or R₅ and R₆, or R₆ and R₇ together with the carbon atoms to which they are attached form a 6-membered aryl, or a 5- or 6-membered cyclic, heterocyclic, or heteroaryl ring optionally substituted with up to three independently selected optional R₂₀ groups;
R₈ is selected from selected from H, nitrogen protecting groups and R₂₀;
X₃ is selected from C═O and C—OH, or Y⁵ is selected from C═O, C—OH and C—NH₂, with the carbon forming part of the ring and
   when X₃ or Y⁵ is C═O then [symbol] represents an α,β-unsaturated double bond conjugated with the C═O; and when X₃ is C—OH or Y⁵ is C—OH or C—NH₂ then [symbol] represents the double bonds of an aromatic 6-membered ring and R₃ is absent;
X₁ is selected from O, S, NR₂₁, CR₂₁R₂₂, CR₂₁R₂₂O, C(═O), C(═O)NR₂₁, NR₂₁C(═O), C(O)—R^{A}—C(O)—NH, C(O)—R^{A}—NH—C(O), C(O)—NH—R^{A}—C(O), NH—C(O)—R^{A}—C(O), NH—C(O)—R^{A}—C(O)—NH, NH—C(O)—R^{A}—NH—C(O), C(O)—NH—R^{A}—NH—C(O), C(O)—NH—R^{A}—C(O)—NH, O—C(O) and C(O)—O or is absent;
L is selected from an amino acid, a peptide chain having from 2 to 12 amino acids, a paraformaldehyde chain —(OCH₂)₁₋₂₄—, a polyethylene glycol chain —(OCH₂CH₂)₁₋₁₂— and —(CH₂)ₘ—Y⁶—(CH₂)ₙ— wherein
   m is an integer selected from 0 to 12,
   n is an integer selected from 0 to 12, and
   Y⁶ is selected from —(CH₂)_{z}— and a group (L1) that is selected from arylene, monocyclic heteroarylene, monocyclic cycloalkylene, monocyclic cycloalkenylene and monocyclic heterocyclylene groups optionally substituted with up to three independently selected optional R₂₀ groups;
   z is an integer selected from 1 to 5;
X₂ is selected from O, S, NR₂₃, CR₂₃R₂₄, CR₂₃R₂₄O, C(═O), C(═O)NR₂₃, NR₂₄C(═O), C(O)—R^{A}—C(O)—NH, C(O)—R^{A}—NH—C(O), C(O)—NH—R^{A}—C(O), NH—C(O)—R^{A}—C(O), NH—C(O)—R^{A}—C(O)—NH, NH—C(O)—R^{A}—NH—C(O), C(O)—NH—R^{A}—NH—C(O), C(O)—NH—R^{A}—C(O)—NH, O—C(O) and C(O)—O or is absent;
B is a polycyclic group selected from:

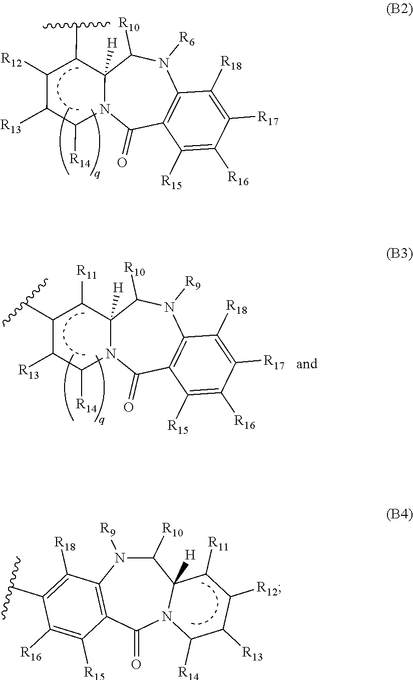

the dotted lines indicate the optional presence of one or more double bonds; q is 0 or 1;
and either:
   (i) R₉ and R₁₀ together form a double bond;
   (ii) R₉ is H and R₁₀ is OH;
   (iii) R₉ is H and R₁₀ is OC₁₋₆ alkyl; or
   (iv) R₉ is selected from SO₃H, nitrogen protecting groups and R₂₀; and R₁₀ is H;
R₁₁, R₁₂, R₁₃ and R₁₄ are independently selected from H, R₂₀, R₂₅, ═CH₂, ═CH—(CH₂)ₛ—CH₃, ═CH—(CH₂)ₛ—R₂₅, ═O, (CH₂)ₛ—OR₂₅, (CH₂)ₛ—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)ₛ—SO₂R₂₅, O—SO₂R₂₅, (CH₂)ₛ—C(O)R₂₅ and (CH₂)ₛ—C(O)NR₂₅R₂₆;
   or one of R₁₁ and R₁₂, R₁₂ and R₁₃, or R₁₃ and R₁₄ together with the carbon atoms to which they are attached form a 6-membered aryl, or a 5- or 6-membered cyclic, heterocyclic, or heteroaryl ring optionally substituted with up to three independently selected optional R₂₀ groups;
each s is an integer independently selected from 0 to 6;
each t is an integer independently selected from 1 to 6;
R₁₅, R₁₆, R₁₇ and R₁₈ are independently selected from H and R₂₀;
each R₂₀ is independently selected from (CH₂)ⱼ—OH, C₁₋₆ alkyl, OC₁₋₆ alkyl, OCH₂Ph, (CH₂)ⱼ—CO₂R₂₇, O—(CH₂)ₖ—NR₂₇R₂₈, (CH₂)ⱼ—NR₂₇R₂₈, C(═O)—NH—(CH₂)ₖ—NR₂₇R₂₈; C(═O)—NH—C₆H₄—(CH₂)ⱼ—R₂₇ and C(═O)—NH—(CH₂)ₖ—C(═NH)NR₂₇R₂₈;
   each j is an integer independently selected from 0 to 6;
   each k is an integer independently selected from 1 to 6;
each R₁₉, R₂₁, R₂₂, R₂₃, R₂₄, R₂₆, R₂₇ and R₂₈ is independently selected from H and C₁₋₆ alkyl; and
each R₂₅ is independently selected from H, C₁₋₁₂ alkyl, C₅₋₉ heteroaryl, C₆₋₁₅ heteroarylalkyl, phenyl and C₇₋₁₂ aralkyl groups; wherein the heteroaryl, heteroarylalkyl, phenyl and aralkyl groups are optionally substituted with up to three independently selected optional R₂₀ groups
each R^{A} is independently selected from:
   —NR^{B}—T¹—NR^{C}— where R^{B} and R^{C} are each independently selected from H and C₁₋₈ alkyl, or together R^{B} and R^{c} join to form a ring and together are (CH₂)₂₋₃, where T¹ is selected from —C(O), —C(O)(CH₂)₀₋₅₀C(O)—, —C(O)PhC(O)— where Ph is 1,3- or 1,4-phenylene;
   -het- wherein het is a mono-, bi-, or tricyclic heteroarylene of 5 to 12 members, containing one, two, or three heteroatoms independently selected from O, N, S, P and B, wherein het is optionally substituted up to three independently selected optional R₂₀ groups;
   -X^{A}-T²—X^{A}-, where T² is:

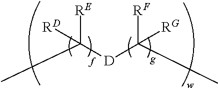

wherein each X^{A} is independently selected from a bond, —NH—, —N(C₁₋ₛ alkyl)-, —O— and —S—,
each R^{D}, R^{E}, R^{F}, and R^{G} are each independently H or R₂₀, or R^{D} and R^{E} form a ring system, or R^{F} and R^{G} form a ring system, or both R^{D} and R^{E}, and R^{F} and R^{G} independently form ring systems, where said ring systems are independently selected from —C₁-C₁₀ heterocyclyl or —C₃-C₈ carbocyclycl, or R^{D}, R^{E}, R^{F}, and R^{G} are each bonds to different carbons on D, wherein f and g are each independently an integer from 0 to 50 and w is an integer from 1 to 50, and wherein D is a bond or is selected from the group consisting of —S—, —C₁-C₅ alkylene-, —C₆-C₁₄ arylene-, —C₆-C₁₄ heteroarylene-, —C₁-C₅ heteroalkylene-, —C₇-C₂₂ aralkylene, —C₁-C₁₀ heterocyclo and —C₃-C₈ carbocyclo, where said —C₁-C₅ alkylene-, —C₆-C₁₄ arylene-, —C₆-C₁₄ heteroarylene-, —C₁-C₅ heteroalkylene-, —C₇-C₂₂ aralkylene, —C₁-C₁₀ heterocyclo and —C₃-C₈ carbocyclo are optionally substituted up to three independently selected optional R₂₀ groups.

In a further aspect, there is provided a compound of formula (I) and salts, solvates and tautomers thereof for use in a method of therapy.

In a further aspect, there is provided a compound of formula (I) and salts, solvates and tautomers thereof for use as a medicament.

In a further aspect, there is provided a compound of formula (I) and salts, solvates and tautomers thereof for use in the treatment of a proliferative disease.

In a further aspect, there is provided a pharmaceutical composition comprising a compound of formula (I) and salts and solvates thereof and a pharmaceutically acceptable carrier or diluent. The pharmaceutical composition of the present invention may further comprise one or more (e.g. two, three or four) further active agents.

In a further aspect, the present invention provides the use of a compound of formula (I) and salts, solvates and tautomers thereof in the manufacture of a medicament for treating a proliferative disease.

In a further aspect, the present invention provides a method of treatment of a patient suffering from a proliferative disease, comprising administering to said patient a therapeutically effective amount of a compound of formula (I) and salts, solvates and tautomers thereof or a pharmaceutical composition of the present invention.

In a further aspect, the compound of formula (I) and salts, solvates and tautomers thereof may be administered alone or in combination with other treatments, either simultaneously or sequentially depending upon the condition to be treated.

In a further aspect, the compound of formula (I) and salts, solvates and tautomers thereof, may be used as a payload on a tumour-targeting agent (e.g., antibody, antibody fragment, hormone, etc.).

### Definitions

The following abbreviations are used throughout the specification: Ac acetyl; AIBN Azobisisobutyronitrile; Alloc allyloxycarbonyl; BAIB bis(acetoxy)iodobenzene/(diacetoxyiodo)benzene; Boc tert-butoxycarbonyl; BPDs benzopyrridodiazecines; CBz benzyloxycarbonyl; CBI cyclopropyl[c]benzo[e]indolone, CPI cyclopropylpyrolo[e]-indolone, DBU 1,8-diazabicyclo[5.4.0]undec-7-ene; DHP dihydropyran; DMAP 4-dimethylaminopyridine; DMF dimethylformamide; DMSO dimethylsulfoxide; DPPA diphenylphosphory azide; EDCl 1-Ethyl-3-(3-dimethylaminopropyl)carbodiimide; Et ethyl; Et₂O diethyl ether; EtOAc ethyl acetate; EtOH ethanol; Fmoc 9-fluorenylmethyl-oxycarbonyl; HATU (1-[Bis(dimethylamino)methylene]-1H-1,2,3-triazolo[4,5-b]-pyridinium 3-oxid hexafluorophosphate); HMDST hexamethyldisilathiane; iBu iso-butyl; KOtBu potassium t-butoxide; L-Selectride Lithium tri-sec-butyl(hydride)borate; Me methyl; MeOH methanol; PBDs pyrrolo[2,1-c][11,4]benzo-diazepines; PDDs pyrridinobenzodiazepines; PIFA phenyliodine (III) bis[trifluoroacetate]; Ph phenyl; p-TSA/PTSA p-Toluenesulfonic acid; Pyr pyridine; TBAF tetrabutylammonium fluoride; TBAI tetrabutylammonium iodide; TBS-Cl/TBDMSCl tert-butyldimethylsilyl chloride; TEA triethylamine; TEMPO (2,2,6,6-tetramethyl-piperidin-1-yl)oxyl; TFA trifluoro-acetic acid; THF tetrahydrofuran; THP tetrahydropyranyl; Troc 2,2,2-Trichloroethyl carbonate and Ts (tosylate) p-toluene sulfonic acid.

“Substituted”, when used in connection with a chemical substituent or moiety (e.g., an alkyl group), means that one or more hydrogen atoms of the substituent or moiety have been replaced with one or more non-hydrogen atoms or groups, provided that valence requirements are met and that a chemically stable compound results from the substitution.

“Optionally substituted” refers to a parent group which may be unsubstituted or which may be substituted with one or more substituents. Suitably, unless otherwise specified, when optional substituents are present the optional substituted parent group comprises from one to three optional substituents. Where a group may be “optionally substituted with up to three groups”, this means that the group may be substituted with 0, 1, 2 or 3 of the optional substituents. Where a group may be “optionally substituted with one or two optional substituents”, this means that the group may be substituted with 0, 1 or 2 of the optional substituents. Suitably groups may be optionally substituted with 0 or 1 optional substituents.

Optional substituents may be selected from C₁₋₇ alkyl, C₂₋₇ alkenyl, C₂₋₇ alkynyl, C₅₋₂₀ aryl, C₃₋₁₀ cycloalkyl, C₃₋₁₀ cycloalkenyl, C₃₋₁₀ cycloalkynyl, C₃₋₂₀ heterocyclyl, C₃₋₂₀ heteroaryl, acetal, acyl, acylamido, acyloxy, amidino, amido, amino, aminocarbonyloxy, azido, carboxy, cyano, ether, formyl, guanidino, halo, hemiacetal, hemiketal, hydroxamic acid, hydroxyl, imidic acid, imino, ketal, nitro, nitroso, oxo, oxycarbonyl, oxycarboyloxy, sulfamino, sulfamyl, sulfate, sulfhydryl, sulfinamino, sulfinate, sulfino, sulfinyl, sulfinyloxy, sulfo, sulfonamido, sulfonamino, sulfonate, sulfonyl, sulfonyloxy, uredio groups.

“Independently selected” is used in the context of statement that, for example, “each R₂₁ and R₂₂ are independently selected from H and C₁₋₆ alkyl, . . . ” and means that each instance of the functional group, e.g. R₂₁, is selected from the listed options independently of any other instance of R₂₁ or R₂₂ in the compound. Hence, for example, H may be selected for the first instance of R₂₁ in the compound; methyl may be selected for the next instance of R₂₁ in the compound; and ethyl may be selected for the first instance of R₂₂ in the compound.

C₁₋₁₂ alkyl: refers to straight chain and branched saturated hydrocarbon groups, generally having from 1 to 12 carbon atoms; more suitably C₁₋₇ alkyl; more suitably C₁₋₆ alkyl; more suitably C₁₋₃ alkyl. Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, n-heptyl, and the like.

“Alkylene” refers to a divalent radical derived from an alkane which may be a straight chain or branched, as exemplified by —CH₂CH₂CH₂CH₂—.

“Monocyclic cycloalkylene” refers to a divalent radical derived from a saturated monocyclic hydrocarbon group (or cycloalkane). The cycloalkylene group may be attached to the rest of the compound at any ring atom unless such attachment would violate valence requirements. Suitably, the monocylic cycloalkylene group is a C₃₋₁₀ cycloalkylene group that is a cycloalkyl group having from 3 to 10 carbon atoms that comprise the ring. Suitably the monocylic cycloalkylene group is a C₃₋₇ cycloalkylene group, more suitably a C₆ cycloalkylene group (i.e. a cyclohexylene group).

“Amino acid” refers to organic compounds containing amine (—NH₂) and carboxyl (—COOH) functional groups, along with a side chain (R group) specific to each amino acid. Each amino acid may be independently selected from any amino acid. Suitably, each amino acid is an alpha amino acid, where the amine and the carboxylic acid groups are attached to the first (alpha-) carbon atom. Suitably each amino acid may be selected from alanine, arginine, asparagine, aspartic acid, citrulline, cysteine, glutamic acid, glutamine, glycine, histidine, isoleucine, leucine, lysine, methionine, phenylalanine, proline, serine, threonine, tryptophan, tyrosine and valine.

“Aryl”: refers to fully unsaturated monocyclic, bicyclic and polycyclic aromatic hydrocarbons having at least one aromatic ring and having a specified number of carbon atoms that comprise their ring members (e.g., 6-membered aryl refers to an aryl group having 6 carbon atoms as ring members and C₆₋₁₄ aryl refers to an aryl group having 6 to 14 carbon atoms as ring members). The aryl group may be attached to a parent group or to a substrate at any ring atom and may include one or more non-hydrogen substituents unless such attachment or substitution would violate valence requirements. Suitably, a C₆₋₁₄ aryl is selected from a C₆₋₁₂ aryl, more suitably, a C₆₋₁₀ aryl. Examples of aryl groups include phenyl.

“Arylene” refers to a divalent radical derived from an aryl group, e.g. —C₆H₄— which is the arylene derived from phenyl.

“C₇₋₁₂ aralkyl” refers to an arylalkyl group having 7 to 12 carbon atoms and comprising an alkyl group substituted with an aryl group. Suitably the alkyl group is a C₁₋₆ alkyl group and the aryl group is phenyl. Examples of C₇₋₁₂ aralkyl include benzyl and phenethyl. In some cases the C₇₋₁₂ aralkyl group may be optionally substituted and an example of an optionally substituted C₇₋₁₂ aralkyl group is 4-methoxylbenzyl.

“C₃-C₈ carbocyclyl” by itself or as part of another term, is a 3-, 4-, 5-, 6-, 7- or 8-membered monovalent, substituted or unsubstituted, saturated or unsaturated non-aromatic monocyclic or bicyclic carbocyclic ring derived by the removal of one hydrogen atom from a ring atom of a parent ring system. Representative C₃-C₈ carbocyclyl include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, cyclooctadienyl, bicyclo(1.1.1.)pentane, and bicyclo(2.2.2.)octane. A C₃-C₈ carbocyclyl group can be optionally substituted.

Halogen: refers to a group selected from F, Cl, Br, and I. Suitably, the halogen is Cl.

“heteroalkyl,” refers to a stable straight or branched chain hydrocarbon, or combinations thereof, fully saturated or containing from 1 to 3 degrees of unsaturation, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Up to two heteroatoms may be consecutive. Heteroalkyl groups typically comprise from 1 to 15 carbon atoms, preferably from 1 to 12 carbon atoms, more preferably from 1 to 8 carbon atoms, and most preferably from 1 to 4 carbon atoms. Heteroalkyl groups may be optionally substituted.

“heteroalkylene” refers to a divalent group derived from heteroalkyl (as discussed above). For heteroalkylene groups, heteroatoms can also occupy either or both of the chain termini. Heteroalkylene groups may be optionally substituted.

“C₅₋₉ heteroaryl”: refers to unsaturated monocyclic or bicyclic aromatic groups comprising from 5 to 9 ring atoms, whether carbon or heteroatoms, of which from 1 to 5 are ring heteroatoms. Suitably, any monocyclic heteroaryl ring has from 5 to 6 ring atoms and from 1 to 3 ring heteroatoms. Suitably each ring heteroatom is independently selected from nitrogen, oxygen, and sulfur. The bicyclic rings include fused ring systems and, in particular, include bicyclic groups in which a monocyclic heterocycle comprising 5 ring atoms is fused to a benzene ring. The heteroaryl group may be attached to a parent group or to a substrate at any ring atom and may include one or more non-hydrogen substituents unless such attachment or substitution would violate valence requirements or result in a chemically unstable compound.

Examples of monocyclic heteroaryl groups include, but are not limited to, those derived from:
N₁: pyrrole, pyridine;
O₁: furan;
S₁: thiophene;
N₁O₁: oxazole, isoxazole, isoxazine;
N₂O₁: oxadiazole (e.g. 1-oxa-2,3-diazolyl, 1-oxa-2,4-diazolyl, 1-oxa-2,5-diazolyl, 1-oxa-3,4-diazolyl);
N₃O₁: oxatriazole;
N₁S₁: thiazole, isothiazole;
N₂: imidazole, pyrazole, pyridazine, pyrimidine, pyrazine;
N₃: triazole, triazine; and,
N₄: tetrazole.

Examples of heteroaryl which comprise fused rings, include, but are not limited to, those derived from:
O₁: benzofuran, isobenzofuran;
N₁: indole, isoindole, indolizine, isoindoline;
S₁: benzothiofuran;
N₁O₁: benzoxazole, benzisoxazole;
N₁S₁: benzothiazole;
N₂: benzimidazole, indazole;
O₂: benzodioxole;
N₂O₁: benzofurazan;
N₂S₁: benzothiadiazole;
N₃: benzotriazole; and
N₄: purine (e.g., adenine, guanine), pteridine;

“5- or 6-membered heteroaryl”: refers to unsaturated monocyclic aromatic groups comprising from 5 or 6 ring atoms, whether carbon or heteroatoms, of which from 1 to 5 are ring heteroatoms. Suitably, any monocyclic heteroaryl ring has from 5 to 6 ring atoms and from 1 to 3 ring heteroatoms. Suitably each ring heteroatom is independently selected from nitrogen, oxygen, and sulfur. The heteroaryl group may be attached to a parent group or to a substrate at any ring atom and may include one or more non-hydrogen substituents unless such attachment or substitution would violate valence requirements or result in a chemically unstable compound. Examples of monocyclic heteroaryl groups include, but are not limited to, those derived from the list given above in relation to the definition for C₅₋₉ heteroaryl.

“heteroarylene” refers to a divalent radical derived from a heteroaryl group (such as those described above) and preferably contain 5-14, 6-14, or 6-20 carbon atoms in addition to one, two or three heteroatoms. Heteroarylenes may be monocyclic, bicyclic, or tricyclic ring systems. Representative heteroarylenes, are not limited to, but may be selected from triazolylene, tetrazolylene, oxadiazolylene, pyridylene, furylene, benzofuranylene, thiophenylene, benzothiophenylene, quinolinylene, pyrrolylene, indolylene, oxazolylene, benzoxazolylene, imidazolylene, benzimidazolylene, thiazolylene, benzothiazolylene, isoxazolylene, pyrazolylene, isothiazolylene, pyridazinylene, pyrimidinylene, pyrazinylene, triazinylene, cinnolinylene, phthalazinylene, quinazolinylene, pyrimidylene, azepinylene, oxepinylene, and quinoxalinylene. Heteroarylenes are optionally substituted.

“Monocyclic heteroarylene” refers to a divalent radical derived from a monocyclic heteroaryl group (in particular those derived from this list of monocyclic heteroaryl groups provided above).

“C₆₋₁₅ heteroarylalkyl” refers to an alkyl group substituted with a heteroaryl group. Suitably the alkyl is a C₁₋₆ alkyl group and the heteroaryl group is C₅₋₉ heteroaryl as defined above. Examples of C₆₋₁₅ heteroarylalkyl groups include pyrrol-2-ylmethyl, pyrrol-3-ylmethyl, pyrrol-4-ylmethyl, pyrrol-3-ylethyl, pyrrol-4-ylethyl, imidazol-2-ylmethyl, imidazol-4-ylmethyl, imidazol-4-ylethyl, thiophen-3-ylmethyl, furan-3-ylmethyl, pyridin-2-ylmethyl, pyridin-2-ylethyl, thiazol-2-ylmethyl, thiazol-4-ylmethyl, thiazol-2-ylethyl, pyrimidin-2-ylpropyl, and the like.

“C₃₋₂₀ heterocyclyl” or “heterocyclo”: refers to saturated or partially unsaturated monocyclic, bicyclic or polycyclic groups having ring atoms composed of 3 to 20 ring atoms, whether carbon atoms or heteroatoms, of which from 1 to 10 are ring heteroatoms. Suitably, each ring has from 3 to 7 ring atoms and from 1 to 4 ring heteroatoms (e.g., suitably C₃₋₅ heterocyclyl refers to a heterocyclyl group having 3 to 5 ring atoms and 1 to 4 heteroatoms as ring members). The ring heteroatoms are independently selected from nitrogen, oxygen, and sulphur.

As with bicyclic cycloalkyl groups, bicyclic heterocyclyl groups may include isolated rings, spiro rings, fused rings, and bridged rings. The heterocyclyl group may be attached to a parent group or to a substrate at any ring atom and may include one or more non-hydrogen substituents unless such attachment or substitution would violate valence requirements or result in a chemically unstable compound.

Examples of monocyclic heterocyclyl groups include, but are not limited to, those derived from:
N₁: aziridine, azetidine, pyrrolidine, pyrroline, 2H-pyrrole or 3H-pyrrole, piperidine, dihydropyridine, tetrahydropyridine, azepine;
O₁: oxirane, oxetane, tetrahydrofuran, dihydrofuran, tetrahydropyran, dihydropyran, pyran, oxepin;
S₁: thiirane, thietane, tetrahydrothiophene, tetrahydrothiopyran, thiepane;
O₂: dioxoiane, dioxane, and dioxepane;
O₃: trioxane;
N₂: imidazoiidine, pyrazolidine, imidazoline, pyrazoline, piperazine:
N₁O₁: tetrahydrooxazole, dihydrooxazole, tetrahydroisoxazole, dihydroisoxazole, morpholine, tetrahydrooxazine, dihydrooxazine, oxazine;
N₁S₁: thiazoline, thiazolidine, thiomorpholine;
N₂O₁: oxadiazine;
O₁S₁: oxathiole and oxathiane (thioxane); and
N₁O₁S₁: oxathiazine.

Examples of substituted monocyclic heterocyclyl groups include those derived from saccharides, in cyclic form, for example, furanoses, such as arabinofuranose, lyxofuranose, ribofuranose, and xylofuranse, and pyranoses, such as aliopyranose, altropyranose, glucopyranose, mannopyranose, gulopyranose, idopyranose, galactopyranose, and talopyranose.

“5- or 6-membered heterocyclic” refers to saturated or partially unsaturated monocyclic examples of “C₃₋₂₀ heterocyclyl” groups. 5- or 6-membered heterocyclic having ring atoms composed of 5 to 6 ring atoms, whether carbon atoms or heteroatoms, of which from 1 to 4 are ring heteroatoms. More suitably, each ring has from 5 to 6 ring atoms and from 1 to 2 ring heteroatoms. The ring heteroatoms are independently selected from nitrogen, oxygen, and sulphur.

“Monocyclic heterocyclylene” refers to a divalent radical derived from a monocyclic heterocyclyl group (in particular those derived from this list of monocyclic heterocyclyl groups provided above).

“Monocyclic cycloalkenylene” refers to a divalent radical derived from a cycloalkyl that contains at least one double bond. Suitably, the cycloalkenylene group comprises one or two double bonds. The cycloalkenylene group may be attached to the rest of the compound at any ring atom unless such attachment would violate valence requirements. Suitably the monocylic cycloalkenylene group is a C₃₋₇ cycloalkenylene group, more suitably a C₆ cycloalkenylene group (i.e. a cyclohexenylene group).

Nitrogen Protecting Groups

Nitrogen protecting groups are well known in the art and are groups that block or protect the nitrogen groups from further reaction. Nitrogen protecting groups are exemplified by carbamates, such as methyl or ethyl carbamate, 9-fluorenylmethyloxy-carbonyl (Fmoc), substituted ethyl carbamates, carbamates cleaved by 1,6-beta-elimination, ureas, amides, peptides, alkyl and aryl derivatives. Carbamate protecting groups have the general formula:

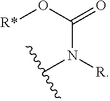

In this specification a zig-zag line indicates the point of attachment of the shown group (e.g. the protecting group above) to the rest of the compound of formula (I). Suitable nitrogen protecting groups may be selected from acetyl, trifluoroacetyl, t-butyloxy-carbonyl (BOC), benzyloxycarbonyl (Cbz) and 9-fluorenylmethyloxy-carbonyl (Fmoc).

A large number of possible carbamate nitrogen protecting groups are listed on pages 706 to 771 of Wuts, P. G. M. and Greene, T. W., Protective Groups in Organic Synthesis, 4^{th} Edition, Wiley-Interscience, 2007, and in P. Kocienski, Protective Groups, 3rd Edition (2005) which are incorporated herein by reference.

Particularly preferred protecting groups include Alloc (allyloxycarbonyl), Troc (2,2,2-Trichloroethyl carbonate), Teoc [2-(Trimethylsilyl)ethoxycarbony], BOC (tert-butyloxycarbonyl), Doc (2,4-dimethylpent-3-yloxycarbonyl), Hoc (cyclohexyloxy-carbonyl), TcBOC (2,2,2-trichloro-tert-butyloxycarbonyl), Fmoc (9-fluorenylmethyloxycarbonyl), 1-Adoc (1-Adamantyloxycarbonyl) and 2-Adoc (2-adamantyloxycarbonyl).

Hydroxyl Protecting Groups

Hydroxyl protecting groups are well known in the art, a large number of suitable groups are described on pages 16 to 366 of Wuts, P. G. M. and Greene, T. W., Protective Groups in Organic Synthesis, 4^{th} Edition, Wiley-lnterscience, 2007, and in P. Kocienski, Protective Groups, 3rd Edition (2005) which are incorporated herein by reference.

Classes of particular interest include silyl ethers, methyl ethers, alkyl ethers, benzyl ethers, esters, benzoates, carbonates, and sulfonates. Particularly preferred protecting groups include THP (tetrahydropyranyl ether).

When trade names are used herein, applicants intend to independently include the trade name product formulation, the generic drug, and the active pharmaceutical ingredient(s) of the trade name product.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgAi, and IgA2. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

By “co-administering” is meant intravenously administering two (or more) drugs during the same administration, rather than sequential infusions of the two or more drugs. Generally, this will involve combining the two (or more) drugs into the same IV bag prior to co-administration thereof.

A drug that is administered “concurrently” with one or more other drugs is administered during the same treatment cycle, on the same day of treatment as the one or more other drugs, and, optionally, at the same time as the one or more other drugs. For instance, for cancer therapies given every 3 weeks, the concurrently administered drugs are each administered on day-1 of a 3-week cycle.

A “chemotherapeutic agent” refers to a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-165 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammail and calicheamicin omegali (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethyl ornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g., celecoxib or etoricoxib), proteosome inhibitor (e.g., PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors; serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARFMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releasing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “epitope” refers to the particular site on an antigen molecule to which an antibody binds.

The “epitope 4D5” or “4D5 epitope” or “4D5” is the region in the extracellular domain of HER2 to which the antibody 4D5 (ATCC CRL 10463) and trastuzumab bind. This epitope is close to the transmembrane domain of HER2, and within domain IV of HER2. To screen for antibodies which bind to the 4D5 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 4D5 epitope of HER2 (e.g. any one or more residues in the region from about residue 550 to about residue 610, inclusive, of HER2 (SEQ ID NO: 39).

The “epitope 2C4” or “2C4 epitope” is the region in the extracellular domain of HER2 to which the antibody 2C4 binds. In order to screen for antibodies which bind to the 2C4 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 2C4 epitope of HER2. Epitope 2C4 comprises residues from domain II in the extracellular domain of HER2. The 2C4 antibody and pertuzumab bind to the extracellular domain of HER2 at the junction of domains I, II and III (Franklin et al. Cancer Cell 5:317-328 (2004)). Anti-HER2 murine antibody 7C2 binds to an epitope in domain I of HER2. See, e.g., PCT Publication No. WO 98/17797. This epitope is distinct from the epitope bound by trastuzumab, which binds to domain IV of HER2, and the epitope bound by pertuzumab, which binds to domain II of HER2. By binding domain IV, trastuzumab disrupts ligand-independent HER2-HER3 complexes, thereby inhibiting downstream signaling (e.g. PI3K/AKT). In contrast, pertuzumab binding to domain II prevents ligand-driven HER2 interaction with other HER family members (e.g. HER3, HER1 or HER4), thus also preventing downstream signal transduction. Binding of MAb 7C2 to domain I does not result in interference of trastuzumab or pertuzumab binding to domains IV and II, respectively, thereby offering the potential of combining a MAb 7C2 ADC with trastuzumab, trastuzumab emtansine (T-DM-1), and/or pertuzumab. Murine antibody 7C2, 7C2.B9, is described in PCT Publication No. WO 98/17797. An anti-HER2 7C2 humanized antibody is disclosed in WO2016/040723 A1.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, MD (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of LI, 50-55 of L2, 89-96 of L3, 31-35B of HI, 50-58 of H2, and 95-102 of H3. (See Almagro and Fransson, Front. Biosci. 13: 1619-1633 (2008).) Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); non-steroidal anti-inflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including SOLU-MEDROL® methylprednisolone sodium succinate, and dexamethasone; dihydrofolate reductase inhibitors such as methotrexate (oral or subcutaneous); anti-malarial agents such as chloroquine and hydroxychloroquine; sulfasalazine; leflunomide; cytokine or cytokine receptor antibodies including anti-interferon-alpha, -beta, or -gamma antibodies, anti-tumor necrosis factor(TNF)-alpha antibodies (infliximab (REMICADE®) or adalimumab), anti-TNF-alpha immunoadhesin (etanercept), anti-TNF-beta antibodies, anti-interleukin-2 (IL-2) antibodies and anti-IL-2 receptor antibodies, and anti-interleukin-6 (IL-6) receptor antibodies and antagonists (such as ACTEMRA™ (tocilizumab)); anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187); streptokinase; transforming growth factor-beta (TGF-beta); streptodornase; RNA or DNA from the host; FK506; RS-61443; chlorambucil; deoxyspergualin; rapamycin; T-cell receptor (Cohen et al, U.S. Pat. No. 5,114,721); T-cell receptor fragments (Offner et al, Science, 251: 430-432 (1991); WO 90/11294; Ianeway, Nature, 341: 482 (1989); and WO 91/01133); BAFF antagonists such as BAFF antibodies and BR3 antibodies and zTNF4 antagonists (for review, see Mackay and Mackay, Trends Immunol, 23: 113-5 (2002) and see also definition below); biologic agents that interfere with T cell helper signals, such as anti-CD40 receptor or anti-CD40 ligand (CD 154), including blocking antibodies to CD40-CD40 ligand (e.g., Durie et al, Science, 261: 1328-30 (1993); Mohan et al, J. Immunol, 154: 1470-80 (1995)) and CTLA4-Ig (Finck et al, Science, 265: 1225-7 (1994)); and T-cell receptor antibodies (EP 340,109) such as T10B9. Some preferred immunosuppressive agents herein include cyclophosphamide, chlorambucil, azathioprine, leflunomide, MMF, or methotrexate.

An “isolated antibody” is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “HER2,” as used herein, refers to any native, mature HER2 which results from processing of a HER2 precursor protein in a cell. The term includes HER2 from any vertebrate source, including mammals such as primates (e.g. humans and cynomolgus monkeys) and rodents (e.g., mice and rats), unless otherwise indicated.

The term also includes naturally occurring variants of HER2, e.g., splice variants or allelic variants. The amino acid sequence of an exemplary human HER2 precursor protein, with signal sequence (with signal sequence, amino acids 1-22) is shown in SEQ ID NO: 64. The amino acid sequence of an exemplary mature human HER2 is amino acids 23-1255 of SEQ ID NO: 64.

The term “HER2-positive cell” refers to a cell that expresses HER2 on its surface. The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CHI, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program

The term “PD-1 axis binding antagonist” refers to a molecule that inhibits the interaction of a PD-1 axis binding partner with either one or more of its binding partner, so as to remove T-cell dysfunction resulting from signaling on the PD-1 signaling axis—with a result being to restore or enhance T-cell function (e.g., proliferation, cytokine production, target cell killing). As used herein, a PD-1 axis binding antagonist includes a PD-1 binding antagonist, a PD-L1 binding antagonist and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is MDX-1106 (nivolumab) described herein. In another specific aspect, a PD-1 binding antagonist is MK-3475 (lambrolizumab) described herein. In another specific aspect, a PD-1 binding antagonist is CT-01 1 (pidilizumab) described herein. In another specific aspect, a PD-1 binding antagonist is AMP-224 described herein.

The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signalling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific aspect, an anti-PD-L1 antibody is YW243.55. S70 described herein. In another specific aspect, an anti-PD-L1 antibody is MDX-1105 described herein. In still another specific aspect, an anti-PD-L1 antibody is MPDL3280A described herein. In still another specific aspect, an anti-PD-L1 antibody is MEDI4736 described herein.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

A “fixed” or “flat” dose of a therapeutic agent herein refers to a dose that is administered to a human patient without regard for the weight (WT) or body surface area (BSA) of the patient. The fixed or flat dose is therefore not provided as a mg/kg dose or a mg/m² dose, but rather as an absolute amount of the therapeutic agent.

A “loading” dose herein generally comprises an initial dose of a therapeutic agent administered to a patient, and is followed by one or more maintenance dose(s) thereof. Generally, a single loading dose is administered, but multiple loading doses are contemplated herein. Usually, the amount of loading dose(s) administered exceeds the amount of the maintenance dose(s) administered and/or the loading dose(s) are administered more frequently than the maintenance dose(s), so as to achieve the desired steady-state concentration of the therapeutic agent earlier than can be achieved with the maintenance dose(s).

A “maintenance” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks, preferably every 3 weeks.

“Infusion” or “infusing” refers to the introduction of a drug-containing solution into the body through a vein for therapeutic purposes. Generally, this is achieved via an intravenous (IV) bag.

An “intravenous bag” or “IV bag” is a bag that can hold a solution which can be administered via the vein of a patient. In one embodiment, the solution is a saline solution (e.g. about 0.9% or about 0.45% NaCl). Optionally, the IV bag is formed from polyolefin or polyvinal chloride.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity.

Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

A “free cysteine amino acid” refers to a cysteine amino acid residue which has been engineered into a parent antibody, has a thiol functional group (—SH), and is not paired as an intramolecular or intermolecular disulfide bridge.

“Drug”, “drug substance”, “active pharmaceutical ingredient”, and the like, refer to a compound (e.g., compounds of Formula (I) and compounds specifically named above) that may be used for treating a subject in need of treatment.

“Excipient” refers to any substance that may influence the bioavailability of a drug, but is otherwise pharmacologically inactive.

“Pharmaceutically acceptable” substances refers to those substances which are within the scope of sound medical judgment suitable for use in contact with the tissues of subjects without undue toxicity, irritation, allergic response, and the like, commensurate with a reasonable benefit-to-risk ratio, and effective for their intended use.

“Pharmaceutical composition” refers to the combination of one or more drug substances and one or more excipients.

The term “subject” as used herein refers to a human or non-human mammal. Examples of non-human mammals include livestock animals such as sheep, horses, cows, pigs, goats, rabbits and deer; and companion animals such as cats, dogs, rodents, and horses.

“Therapeutically effective amount” of a drug refers to the quantity of the drug or composition that is effective in treating a subject and thus producing the desired therapeutic, ameliorative, inhibitory or preventative effect. The therapeutically effective amount may depend on the weight and age of the subject and the route of administration, among other things.

“Treating” refers to reversing, alleviating, inhibiting the progress of, or preventing a disorder, disease or condition to which such term applies, or to reversing, alleviating, inhibiting the progress of, or preventing one or more symptoms of such disorder, disease or condition.

“Treatment” refers to the act of “treating”, as defined immediately above.

As used herein the term “comprising” means “consisting at least in part of”. When interpreting each statement in this specification that includes the term “comprising”, features other than that or those prefaced by the term may also be present. Related terms such as “comprise” and “comprises” are to be interpreted in the same manner.

A

A is a group selected from:

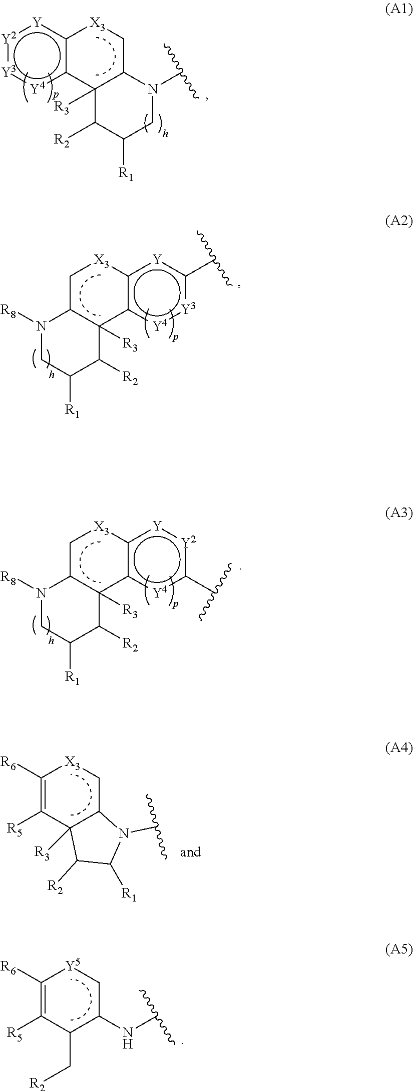

The ring containing Y in (A1), (A2) and (A3) is an aromatic ring and because of the limitations on the substituents is either a 6-membered aryl ring (when p is 1) or is a 5-membered heteroaryl ring (when p is 0).

Thus, when p is 1 (A1), (A2) and (A3) may be represented by (A6), (A7) and (A8):

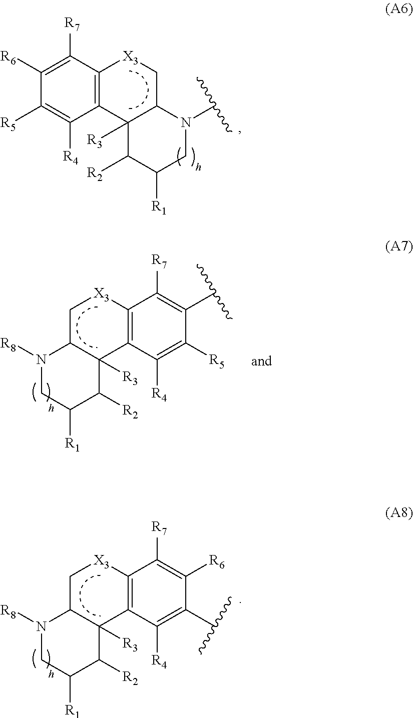

When p is 0 (A1), (A2) and (A3) may be represented by (A9), (A10), (A11), (A12) and (A13):

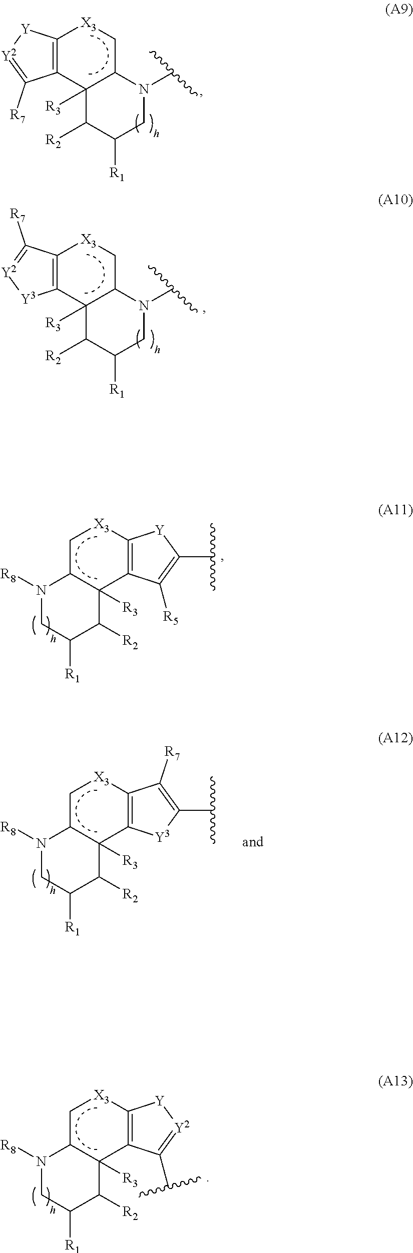

Suitably A is selected from (A4), (A5), (A6), (A7), (A8), (A9), (A10), (A11), (A12) and (A13).

In (A1), (A2), (A3), (A4) and (A5) when X₃ or Y⁵ is C═O with the carbon forming part of the ring, then [symbol] represents an α,β-unsaturated double bond conjugated with the C═O such that (A1), (A2), (A3), (A4) and (A5) are represented by (A14), (A15), (A16), (A17) and (A18) respectively:

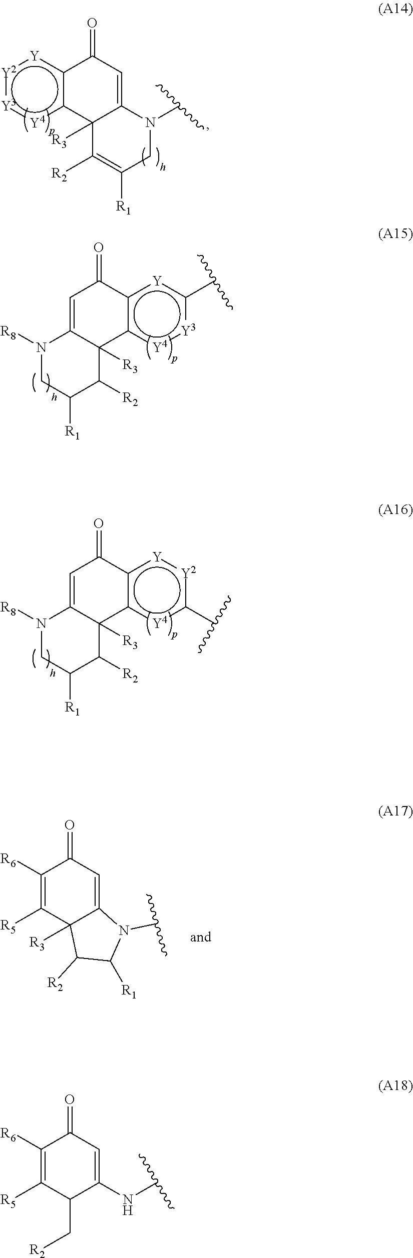

In (A1), (A2), (A3), (A4) and (A5) when X₃ is C—OH or Y⁵ is C—OH or C—NH₂ then [symbol] represents the double bonds of an aromatic 6-membered ring and R₃ is absent such that (A1), (A2), (A3), (A4) and (A5) are represented by (A19), (A20), (A21), (A22) and (A23) respectively:

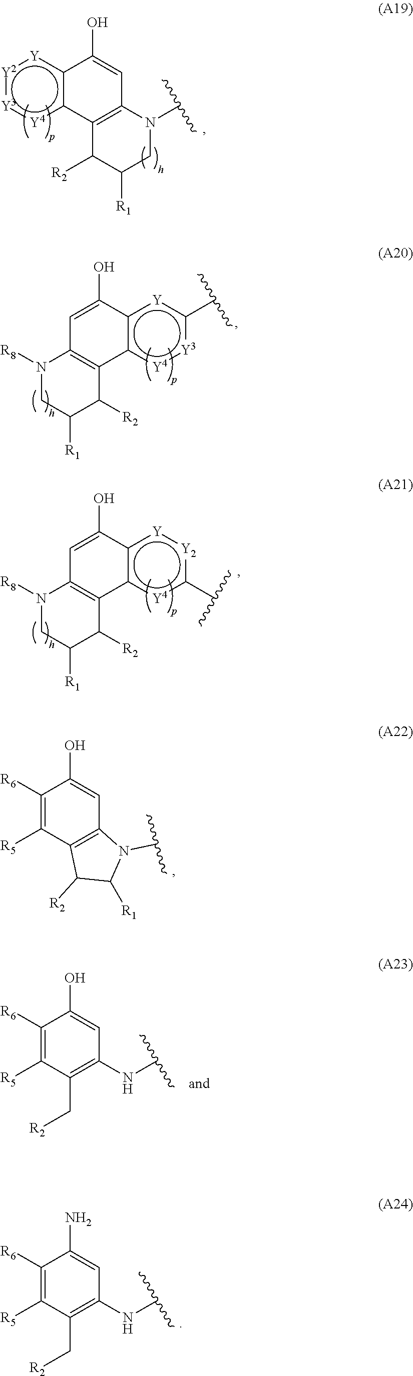

Suitably A is selected from (A4), (A5), (A6), (A7), (A8), (A9), (A10), (A11), (A12), (A13), (A14), (A15), (A16), (A17), (A18), (A19), (A20), (A21), (A22), (A23) and (A24).

Suitably A is selected from (A1), (A2), (A3) and (A4). Suitably A is selected from (A1), (A2) and (A3).

Suitably A is (A1). Suitably, (A1) is selected from:

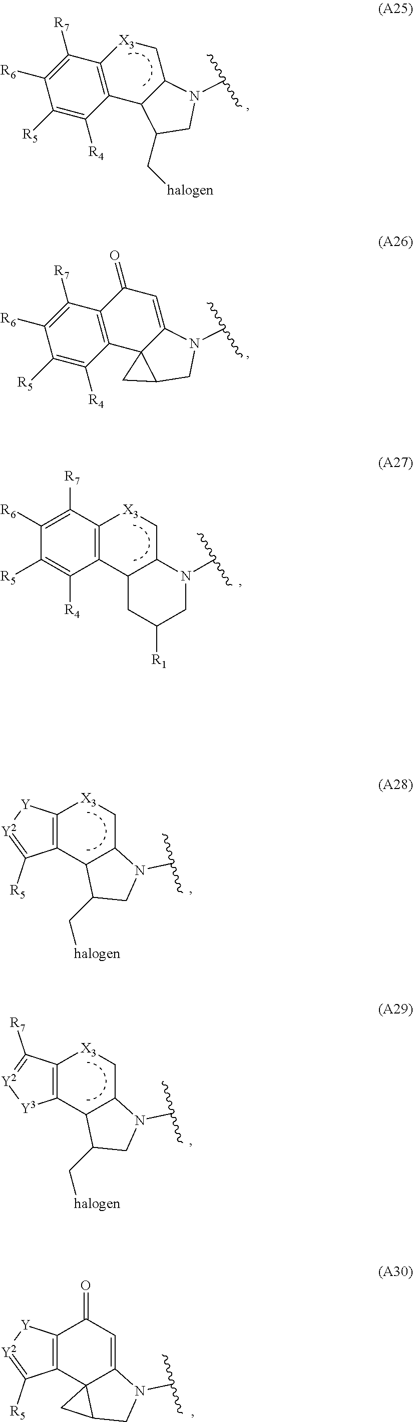

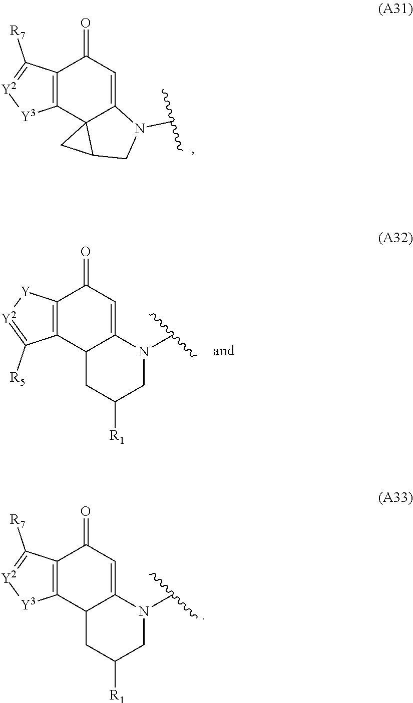

Hence, when p is 1 then (A1) is suitably selected from (A25), (A26) and (A27); and when p is 0 then (A1) is suitably selected from (A28), (A29), (A30), (A31), (A32) and (A33). More suitably, (A1) is selected from (A25), (A26), (A27) (A28), (A29), (A30) and (A31).

Suitably A is (A2). Suitably, (A2) is selected from:

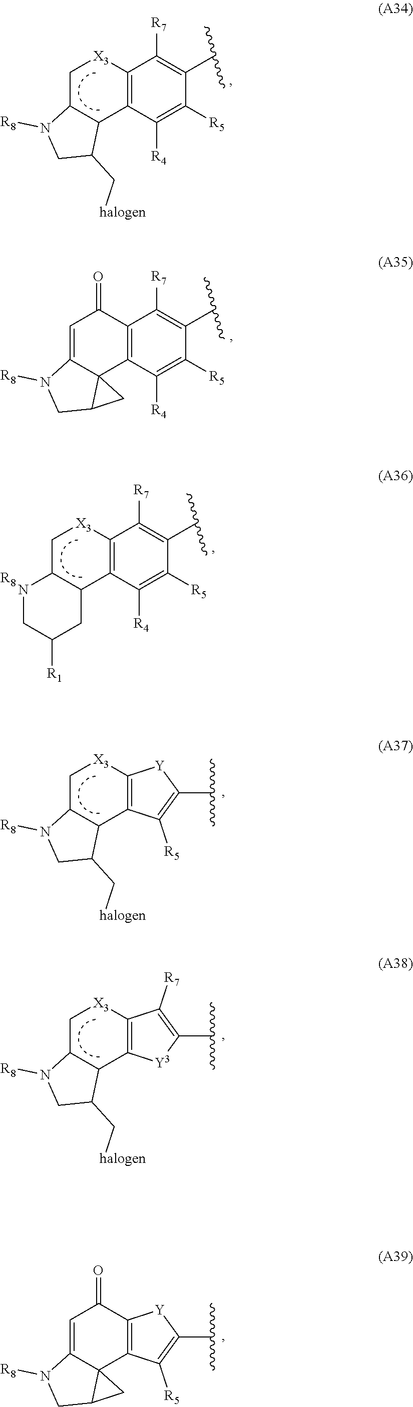

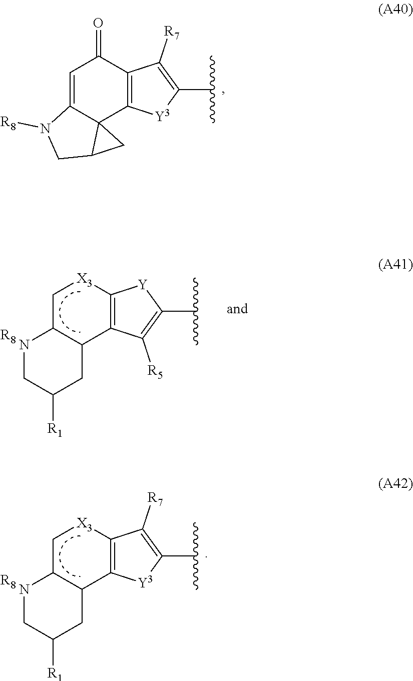

More suitably, (A2) is selected from (A34), (A35), (A37), (A38), (A39) and (A40).

Suitably A is (A3). Suitably, (A3) is selected from:

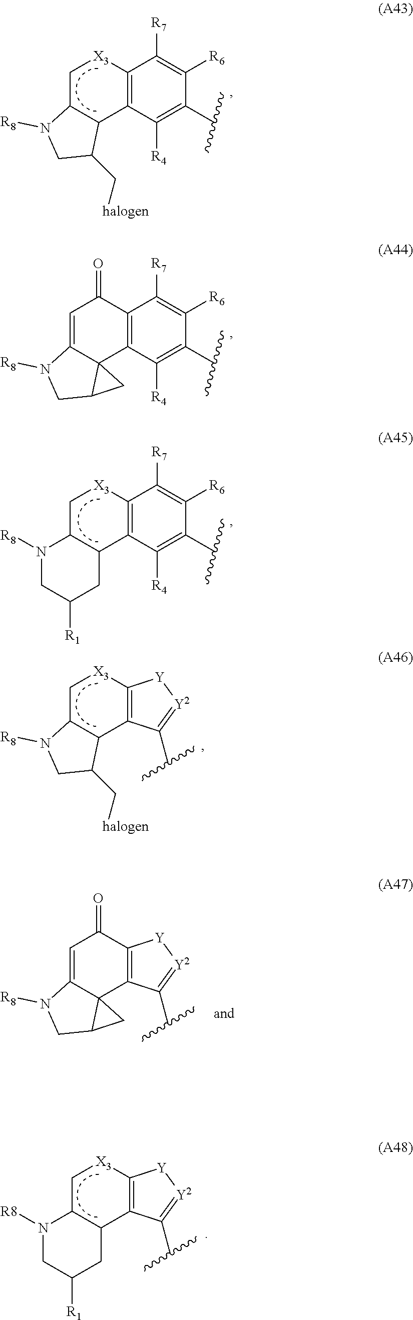

More suitably, (A3) is selected from (A43), (A44), (A46) and (A47).

Suitably A is (A4). Suitably, (A4) is selected from:

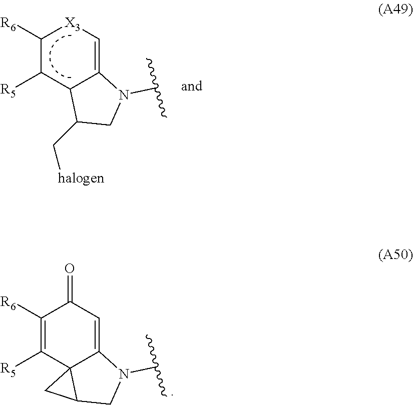

Suitably A is (A5). Suitably, (A5) is:

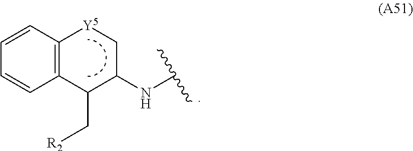

More suitably, A is selected from:

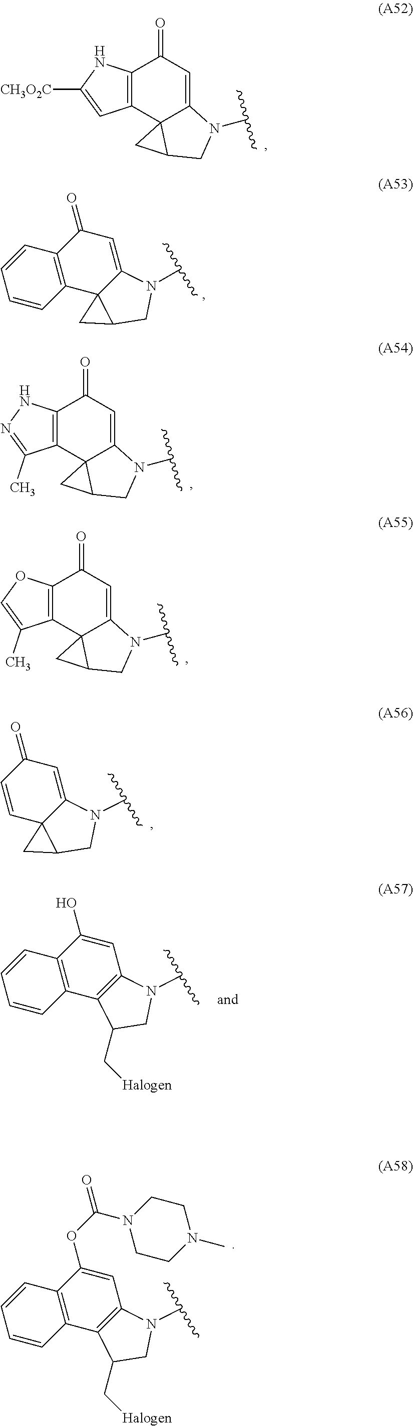

In one aspect, A is selected from

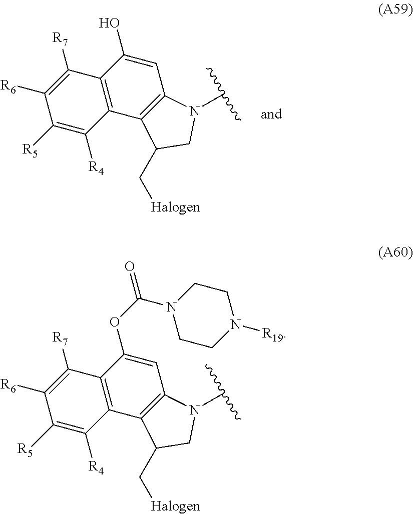

X₁

Suitably, X₁ is selected from O, S, NR₂₁, CR₂₁R₂₂, CR₂₁R₂₂O, C(═O), C(═O)NR₂₁, NR₂₁C(═O), O—C(O) and C(O)—O or is absent.

Suitably, X₁ is selected from O, S, NR₂₈, CR₂₈R₂₉, C(═O), C(═O)NR₂₈ and NR₂₈C(═O) or is absent. Hence, X₁ may be an ester that links group A to group L in either direction.

Thus, when X₁ is selected as C(═O)NR₂₈ then A is linked to L as follows: A-C(═O)NR₂₈-L-X₂-D, whereas when X₁ is NR₂₈C(═O) then A is linked to L as follows: A-NR₂₈C(═O)—L-X₂-D.

Suitably, X₁ is selected from C(═O), C(═O)NH and NHC(═O) or is absent.

Most suitably, when A is (A1) then X₁ is C(═O).

Most suitably, when A is (A2) then X₁ is NHC(═O).

L

Suitably, L is selected from —(CH₂)ₘ—(CH₂)_{z}—(CH₂)ₙ—,

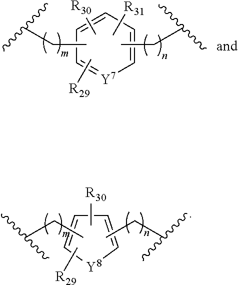

The above structures are drawn without specifying the positions of any of the groups, i.e. groups R₂₉, R₃₀, R₃₁, and the two groups (shown by bonds that end in a zig-zag line) where the ring is attached to the rest of the molecule. Hence, these groups may be present on any position of the ring except for Y⁷ or Y⁸ (as positioning a group, such as R₂₉ at Y⁷ or Y⁸ would not meet the valence requirements).

Suitably, L is selected from —(CH₂)ₘ—(CH₂)_{z}—(CH₂)ₙ—,

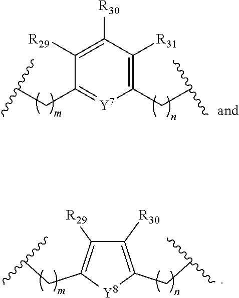

Suitably, L is selected from —(CH₂)₀₋₁₀—(CH₂)₁₋₅—(CH₂)₀₋₁₀— and

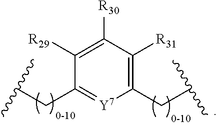

Suitably, L is selected from —(CH₂)₀₋₅—(CH₂)₁₋₅—(CH₂)₀₋₅— and

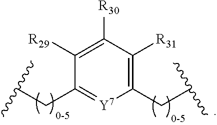

Suitably, L is selected from —(CH₂)₀₋₃—(CH₂)₁₋₃—(CH₂)₀₋₃— and

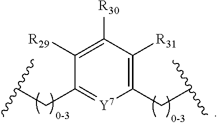

More suitably, L is selected from —CH₂—, —CH₂—CH₂—, —CH₂—CH₂—CH₂—, —CH₂—CH₂—CH₂—CH₂—, —CH₂—CH₂—CH₂—CH₂—CH₂—, —CH₂—CH₂—CH₂—CH₂—CH₂—CH₂—, —CH₂—CH₂—CH₂—CH₂—CH₂—CH₂—CH₂— and

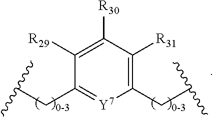

In one aspect, L is

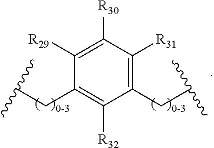

X₂

Suitably, X₂ is selected from O, S, NR₂₃, CR₂₃R₂₄, CR₂₃R₂₄O, C(═O), C(═O)NR₂₃, NR₂₄C(═O), O—C(O) and C(O)—O or is absent.

Suitably, X₂ is selected from O, S, CR₃₀R₃₁, C(═O), C(═O)NR₃₀, NR₃₀C(═O) or is absent.

Hence, X₂ may be an ester that links group L to group B in either direction. Thus, when X₂ is selected as C(═O)NR₃₀ then L is linked to B as follows: A-X₁-L-C(═O)NR₃₀-D, whereas when X₂ is NR₃₀C(═O) then L is linked to B as follows: A-X₁-L-NR₃₀C(═O)—D.

Suitably, X₂ is selected from 0, CR₃₀R₃₁, C(═O) or is absent.

Most suitably, when B is (B1) then X₂ is O.

Most suitably, when B is (B2) then X₂ is CR₃₀R₃₁ or is absent.

B

B is a polycyclic group selected from:

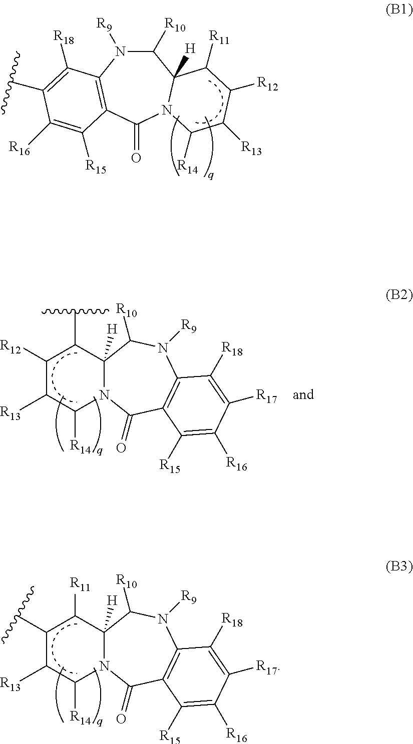

When q is 1, the C-ring is a 6-membered ring that contains the C—R₁₄ group. However, when q is 0, the C—R₁₄ group in the brackets is removed and the C-ring is a 5-membered ring.

Suitably, B is a polycyclic group selected from:

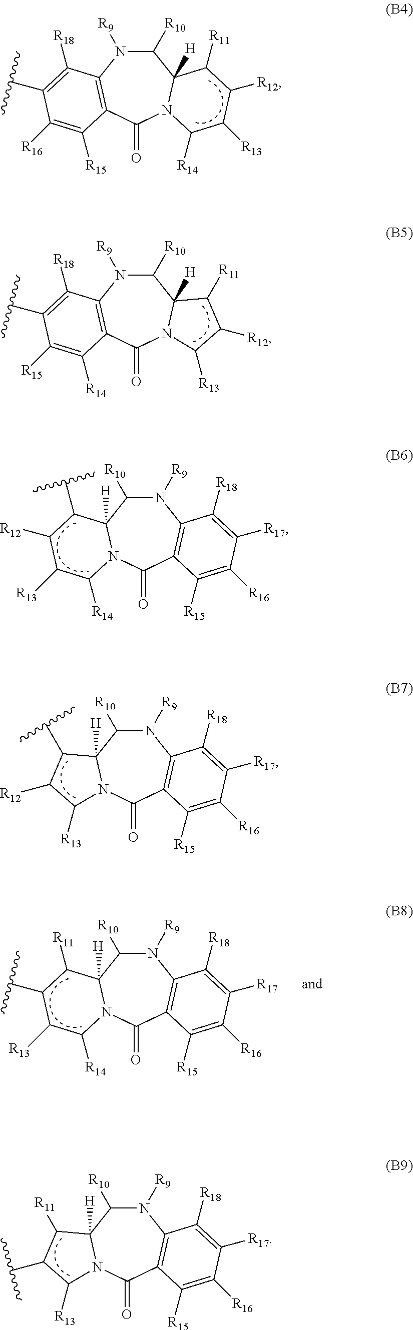

In some aspects, B is a polycyclic group selected from (B2), (B3) and (B4). Suitably, B is selected from (B4), (B6), (B7), (B8) and (B9). Suitably, B is selected from (B4), (B6) and (B8).

In some aspects, B is (B1). Suitably, (B1) is selected from:

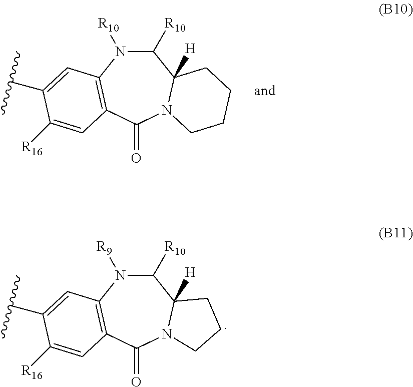

More suitably, (B1) is (B10).

In some aspect, B is (B2). Suitably, (B2) is selected from:

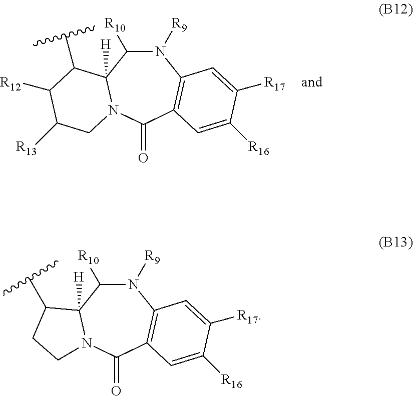

In some aspect, B is (B3). Suitably, (B3) is selected from:

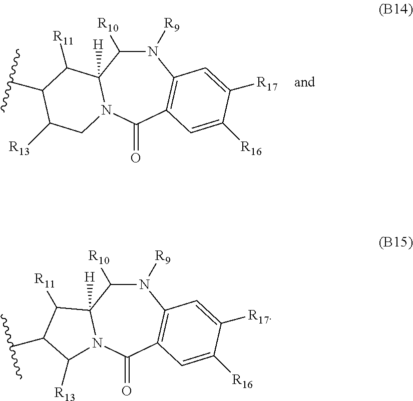

More suitably, B is selected from:

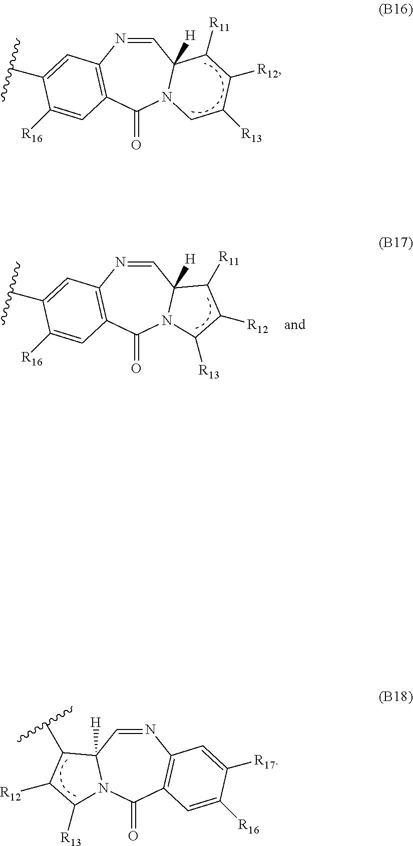

Optional Double Bonds in the C-Ring of Group B

The compounds of formula (I) comprise a group B selected from (B1), (B2) and (B3);

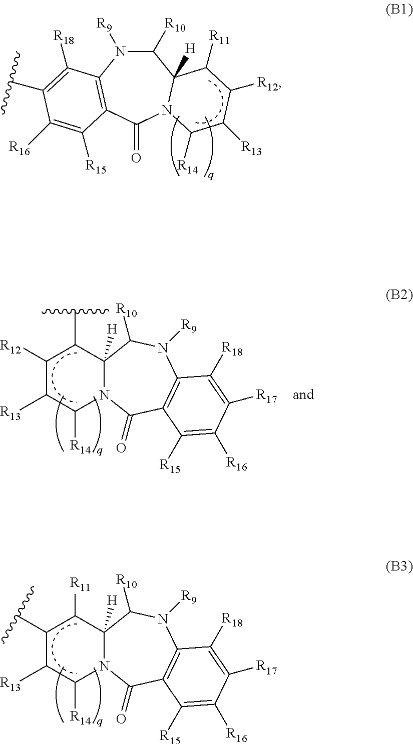

wherein the dotted lines indicate the optional presence of one or more double bonds.

Hence, when q=1 the compounds of formula (I) may be fully saturated or may optionally have one or two double bonds. When q=1, if one double bond is present it may be situated between any one of C1 and C2, C2 and C3, and C3 and C4. When q=1 if two double bonds are present they are situated between C1 and C2, and C3 and C4.

In one aspect, B is (B1), q is 1 and (B1) comprises one or more optional double bonds and is selected from (B19), which has a double bond between C1 and C2; (B20), which has a double bond between C2 and C3; (B21), which has a double bond between C3 and C4; and (B22) which has a double bond between C1 and C2 and a second double bond between C3 and C4:

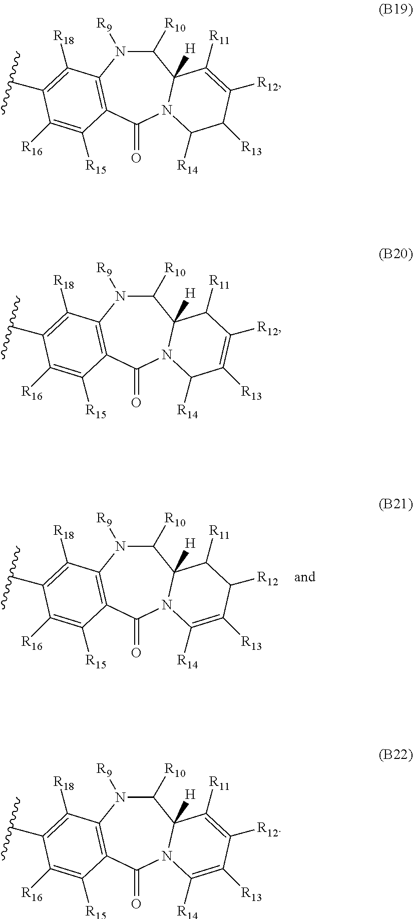

In another aspect, B is (B2), q is 1 and (B2) comprises one or more optional double bonds and is selected from:

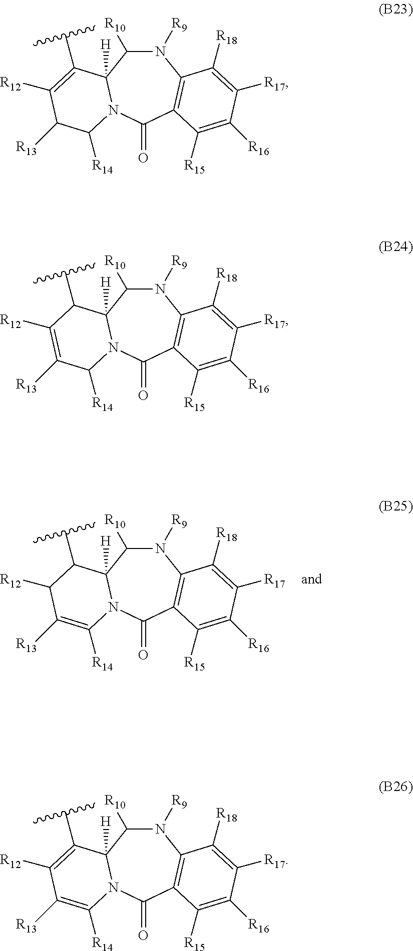

In another aspect, B is (B3), q is 1 and (B3) comprises one or more optional double bonds and is selected from:

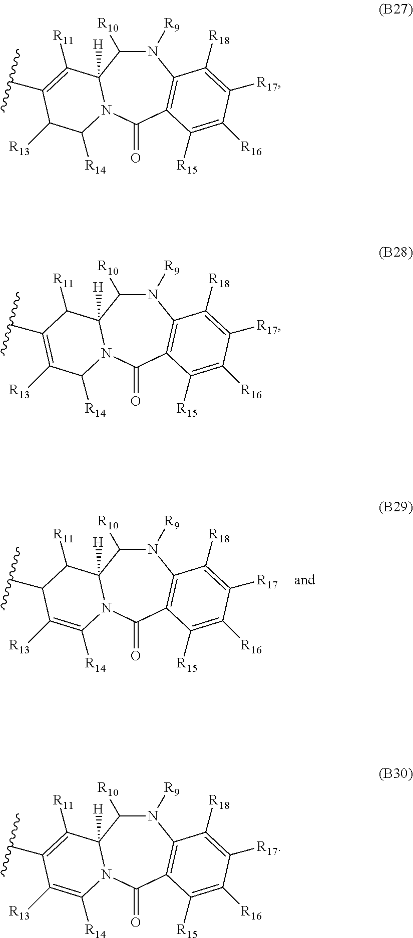

Hence, when q=0 the compounds of formula (I) may be fully saturated or may optionally have one double bond. When q=0 if a double bond is present it is situated between C1 and C2 or C2 and C3.

In another aspect, B is (B1), q is 0 and (B1) comprises an optional double bond and is selected from (B31), which has a double bond between C1 and C2; and (B32), which has a double bond between C2 and C3;

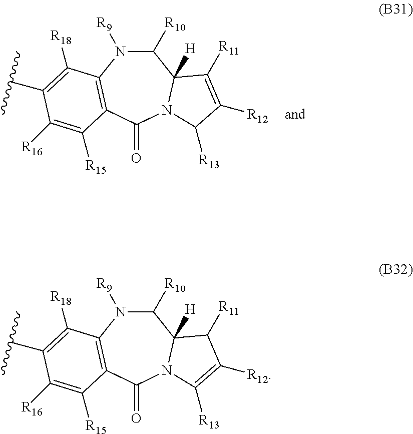

In another aspect, B is (B2) q is 0 and (B2) comprises an optional double bond and is selected from:

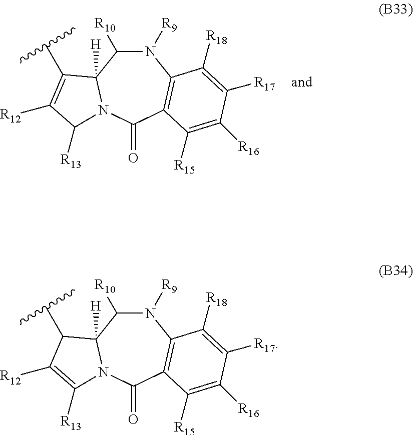

In another aspect, B is (B2) q is 0 and (B2) comprises an optional double bond and is selected from:

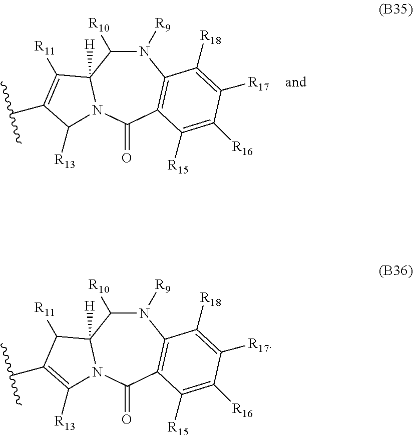

In another aspect, B is selected from:

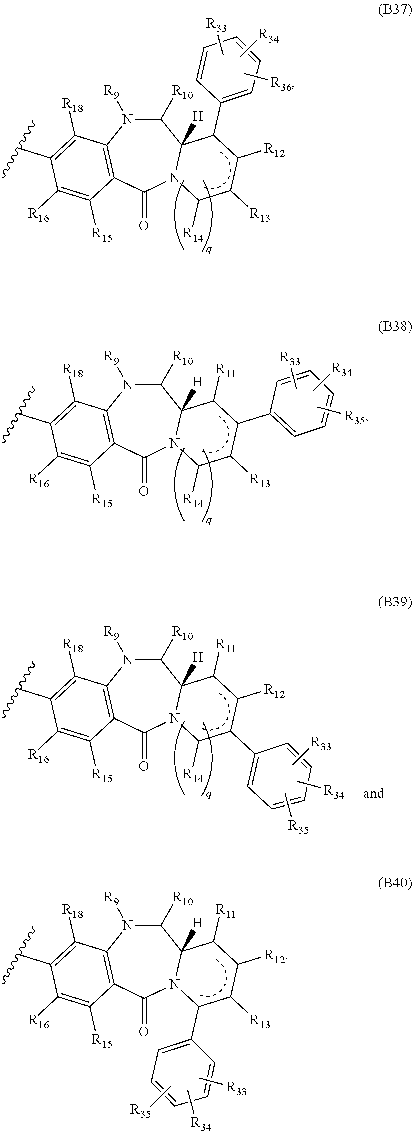

Suitable Structures

Suitably the compound of formula (I) is a compound that has the formula (II):

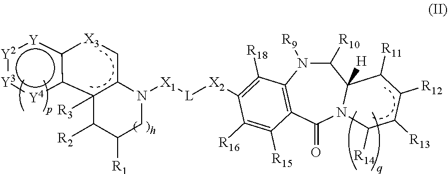

and salts, solvates and tautomers thereof.

Suitably the compound of formula (II) is a compound that has the formula (III):

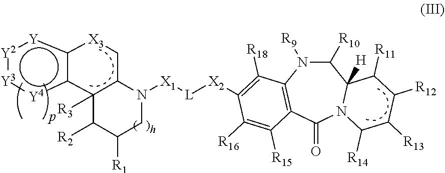

and salts, solvates and tautomers thereof.

Suitably the compound of formula (II) is a compound that has the formula (IV):

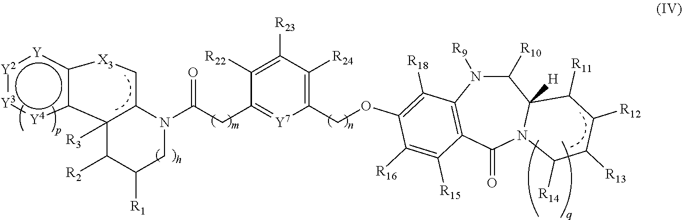

and salts, solvates and tautomers thereof.

Suitably the compound of formula (II) is a compound that has the formula (V):

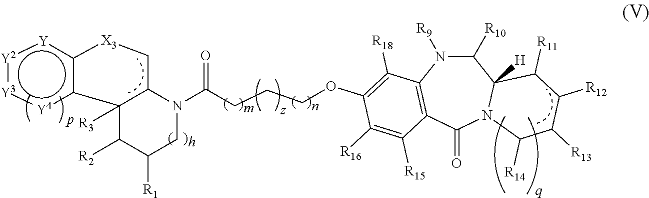

and salts, solvates and tautomers thereof.

Suitably the compound of formula (I) is a compound that has the formula (VI):

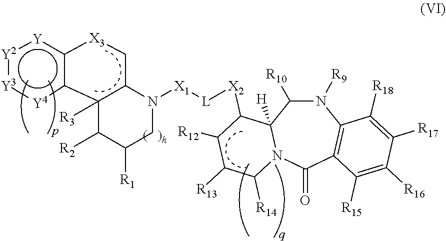

and salts, solvates and tautomers thereof.

Suitably the compound of formula (VI) is a compound that has the formula (VII):

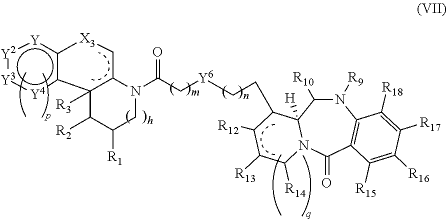

and salts, solvates and tautomers thereof.

Suitably the compound of formula (I) is a compound that has the formula (VIII):

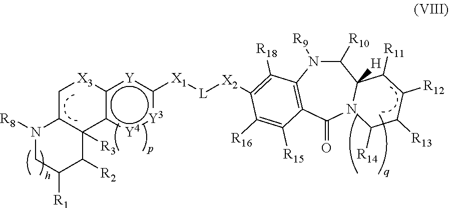

and salts, solvates and tautomers thereof.

Suitably the compound of formula (I) is a compound that has the formula (IX):

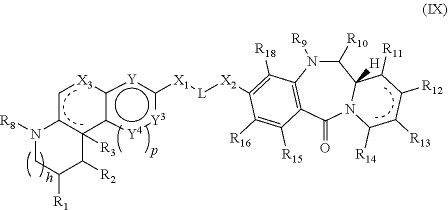

and salts, solvates and tautomers thereof.

Suitably the compound of formula (VIII) is a compound that has the formula (X):

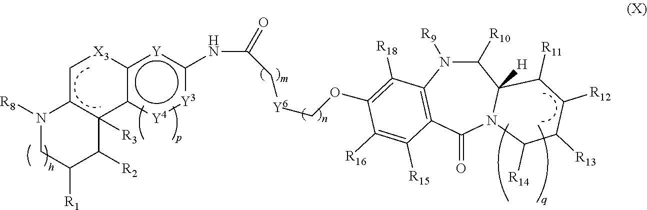

and salts, solvates and tautomers thereof.

Suitably the compound of formula (I) is a compound that has the formula (XI):

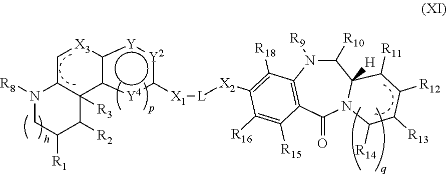

and salts, solvates and tautomers thereof.

Suitably the compound of formula (I) is a compound that has the formula (XII):

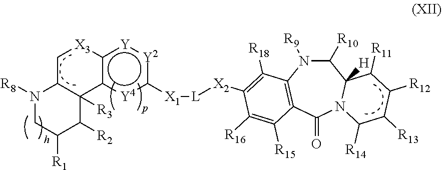

and salts, solvates and tautomers thereof.

Suitably the compound of formula (XI) is a compound that has the formula (XIII):

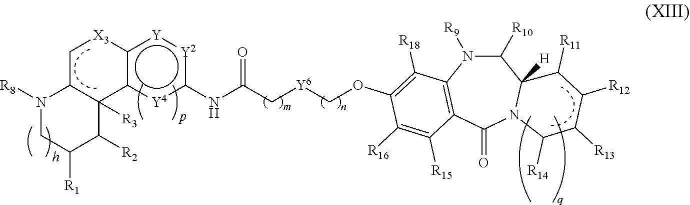

and salts, solvates and tautomers thereof.

Suitably the compound of formula (I) is a compound that has the formula (XIV):

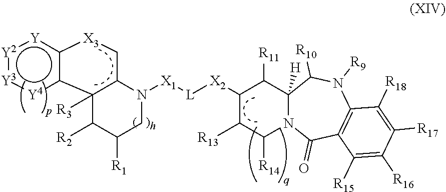

(XIV) and salts, solvates and tautomers thereof.

Suitably the compound of formula (I) is a compound that has the formula (XV):

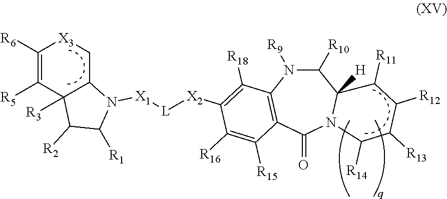

and salts, solvates and tautomers thereof.

Suitably the compound of formula (I) is a compound that has the formula (XVI):

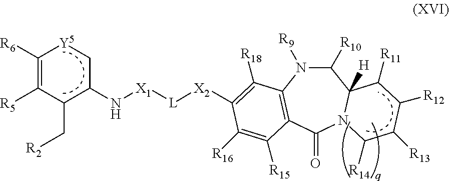

and salts, solvates and tautomers thereof.

Suitably the compound of formula (I) is selected from compounds of the formula (II), (III), (IV), (V), (VI), (VII), (VIII), (IX), (X), (XI) (XII), (XIII), (XIV), (XV) and (XVI) and salts, solvates and tautomers thereof.

X₃

X₃ is selected from C═O; C—OH; and C—R′″ wherein R′″ is a prodrug moiety containing carbonyl, carbamoyl, glycosyl, O-amino, O-acylamino, para-aminobenzyl ether, peptidyl or phosphate groups. Hence, the C of these groups C═O; C—OH; and C—R′″ is a carbon of the ring system of (A1), (A2), (A3) and (A4) in which X₃ appears and the groups ═O; —OH; and —R′″ are substituent groups attached to the ring carbon. For example, for (A1) the X₃ groups C═O; C—OH; C—O—C(═O)—NR′R″; and C—R′″ result in the following structures:

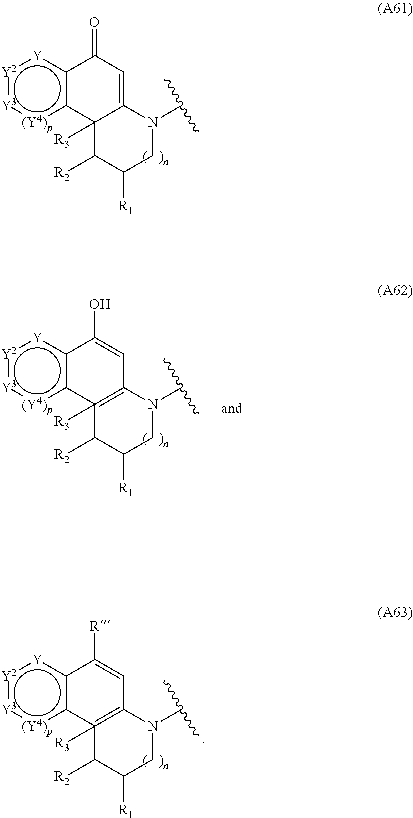

Suitably, X₃ is selected from C═O and C—OH.

R₁

Suitably, R₁ is selected from H, F, Cl, Br and I. More suitably, R₁ is selected from H and C1. More suitably, R₁ is H.

R₂ and R₃

In one aspect, R₂ is —CH₂-halogen and R₃ is H. Suitably in this aspect R₂ is selected from —CH₂—F, —CH₂—Cl, —CH₂—Br and —CH₂—I. More suitably, R₂ is selected from —CH₂—Cl and —CH₂—Br. Most suitably, R₂ is —CH₂—Cl.

In another aspect, R₂ is C₁₋₆ alkyl and R₃ is H. Suitably in this aspect, R₂ is methyl, ethyl, propyl.

In another aspect, R₂ and R₃ together with the carbon atoms to which they are attached form a cyclopropyl ring.

Y

In some aspects, Y is selected from N—R₁₉, O and S. In these aspects, more suitably Y is selected from N—R₁₉ and O. Most suitably, Y is N—R₁₉.

Y²

In some aspects, Y² is selected from C—R₆ and N. More suitable Y² is C—R₆.

Y³

In some aspects, Y³ is selected from N—R₁₉, O and S. In these aspects, more suitably Y³ is selected from N—R₁₉ and O. Most suitably, Y³ is N—R₁₉.

Y⁴

Suitably, Y⁴ is CH.

Y⁵

Y⁵ is selected from C═O; C—OH; C—NH₂; and C—R′″ wherein R′″ is a prodrug moiety containing carbonyl, carbamoyl, glycosyl, O-amino, O-acylamino, para-aminobenzyl ether, peptidyl or phosphate groups. Hence, the C of these groups C═O; C—OH; C—NH₂; and C—R′″ is a carbon of the ring system of (A5) in which Y⁵ appears and the groups ═O; —OH; and —R′″ are substituent groups attached to the ring carbon. Thus, for (A5) the Y⁵ groups C═O; C—OH; C—NH₂; C—O—C(═O)—NR′R″; and C—R′″ result in the following structures:

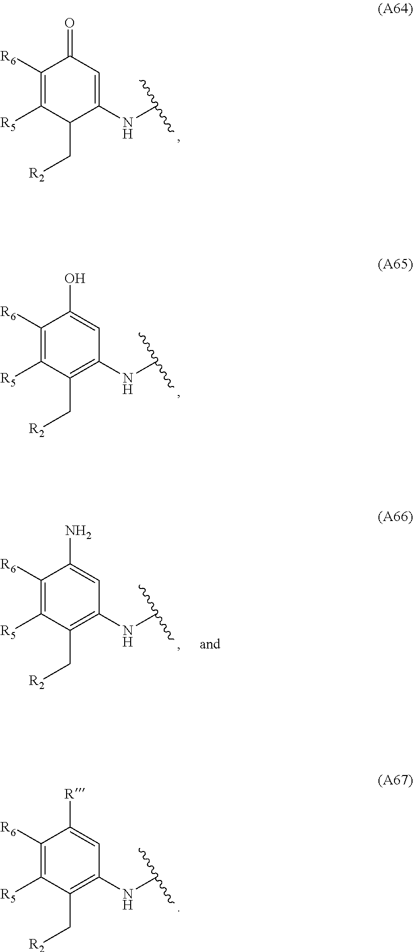

Suitably, Y⁵ is selected C═O; C—OH and C—NH₂.

More suitably, Y⁵ is C—OH.

Y⁶

Suitably, Y⁶ is selected from —(CH₂)_{z}— and a group (L1) that is selected from arylene and monocyclic heteroarylene optionally substituted with up to three independently selected optional R₂₀ groups.

Suitably, Y⁶ is selected from —(CH₂)_{z}— and a group (L1) that is selected from phenylene, pyridinylene, pyrrolylene, pyridylene, furanylene, thiphenylene optionally substituted with up to three independently selected optional R₂₀ groups.

Suitably, Y⁶ is selected from —CH₂—, —CH₂—CH₂—, —CH₂—CH₂—CH₂—, —CH₂—CH₂—CH₂—CH₂—, —CH₂—CH₂—CH₂—CH₂—CH₂— and a group (L1) that is selected from (L2) and (L3); wherein (L2) and (L3) have the following structures:

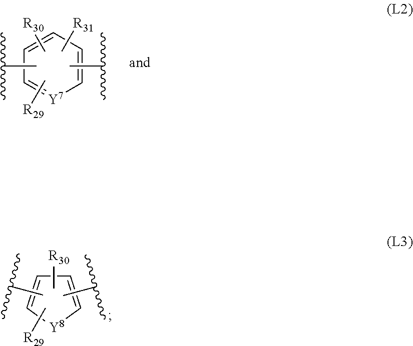

wherein Y⁷ is selected from C—R₃₂ and N;
Y⁸ is selected from N—R₂₅, O and S; and
R₂₉, R₃₀, R₃₁ and R₃₃ are independently selected from H and R₂₀.

Suitably, Y⁶ is selected from —CH₂—, —CH₂—CH₂—, —CH₂—CH₂—CH₂—, —CH₂—CH₂—CH₂—CH₂—, —CH₂—CH₂—CH₂—CH₂—CH₂— and a group (L1) that is selected from (L4) and (L5); wherein (L3) and (L4) have the following structures:

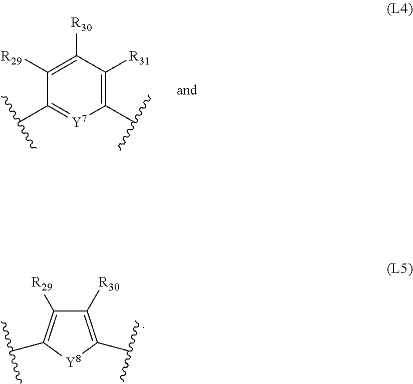

Suitably, Y⁶ is selected from —CH₂—, —CH₂—CH₂—, —CH₂—CH₂—CH₂—, —CH₂—CH₂—CH₂—CH₂—, —CH₂—CH₂—CH₂—CH₂—CH₂— and a group (L1) that has the following structure:

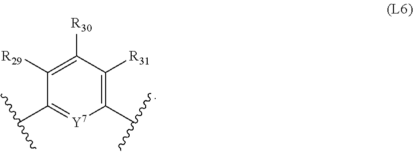

Suitably, Y⁶ is selected from —CH₂—, —CH₂—CH₂—, —CH₂—CH₂—CH₂—, —CH₂—CH₂—CH₂—CH₂—, —CH₂—CH₂—CH₂—CH₂—CH₂— and a group (L1) that has the following structure (L7):

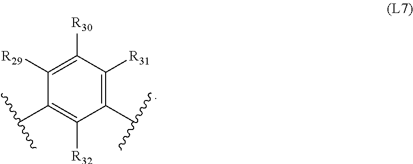

Y⁷

Y⁷ is selected from C—R₂₅ and N.

In one aspect, Y⁷ is C—R₂₅; suitably, Y⁷ is CH.

In another aspect, Y⁷ is N.

Y⁸

Y⁸ is selected from N—R₂₅, O and S.

Suitably, Y⁸ is N—R₂₅; more suitably, Y⁸ is selected from N—H and N—CH₃.

R₄, R₅, R₆ and R₇

In the aspects where one of R₄ and R₅, R₅ and R₆, or R₆ and R₇ together with the carbon atoms to which they are attached form a 6-membered aryl, or a 5- or 6-membered cyclic, heterocyclic, or heteroaryl ring optionally substituted with up to three independently selected optional R₂₀ groups, groups (A1)-(A5) contain a further fused ring (not drawn). In these aspects, then the remaining groups (from R₄, R₅, R₆ and R₇) that do not form the further fused ring are each independently selected from the normal specified list of groups, i.e. from H and R₂₀. For example, where A is (A1), p is 1 and R₅ and R₆ together with the carbon atoms to which they are attached form a 6-membered aryl ring the structure of the group A can be shown as follows:

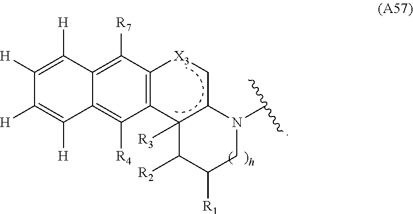

Groups R₄ and R₇ do not form the further fused ring and so are each independently selected from the normal specified list of groups for R₄, R₅, R₆ and R₇, i.e. from H and R₂₀. In addition, the H groups shown on the further fused ring of (A57) may be optionally substituted with up to three independently selected optional R₂₀ groups.

R₈

Suitably, Rₛ is selected from H and R₂₀.

R₉ and R₁₀

Suitably, R₉ and R₁₀ together form a double bond.

In one aspect (ii), R₉ is H and R₁₀ is OH.

In another aspect (iii), suitably R₉ is H and R₁₀ is OCH₃ or OCH₂CH₃.

In another aspect (iv), suitably R₉ is selected from OH, SO₃H, nitrogen protecting groups, methyl, ethyl, OCH₃, OCH₂CH₃, OCH₂Ph, (CH₂)ₛ—CO₂H, (CH₂)ₛ—CO₂CH₃, (CH₂)ₛ—CO₂CH₂CH₃, O—(CH₂)ₜ—NH₂, O—(CH₂)ₜ—NH—CH₃, (CH₂)ₛ—NH₂, (CH₂)ₛ—NH—CH₃, C(═O)—NH—(CH₂)ₜ—NH₂, C(═O)—NH—(CH₂)ₜ—NH—CH₃, C(═O)—NH—C₆H₄—(CH₂)ₛ—H, C(═O)—NH—(CH₂)ₜ—C(═NH)NH₂ and C(═O)—NH—(CH₂)ₜ—C(═NH)NH—CH₃ and R₁₀ is H. More suitably in this aspect (iv), R₉ is selected from OH, SO₃H, methyl, ethyl, OCH₃, OCH₂CH₃, CO₂H, CO₂CH₃, CO₂CH₂CH₃, O—(CH₂)ₜ—NH₂ and (CH₂)ₛ—NH₂ and R₁₀ is H.

In some aspects, R₉ is SO₃H and the compound of formula (I) is a salt thereof. Suitably, in this aspect, R₉ is SO₃H and the compound of formula (I) is an alkali metal salt thereof (AM)+; hence, in this aspect, R₉ may be written as SO₃⁻(AM)⁺. Suitably, R₉ is SO₃H and the compound of formula (I) is an alkali metal salt thereof chosen from Li⁺, Na⁺ and K⁺. More suitably, R₉ is SO₃H and the compound of formula (I) is a Na⁺ salt thereof; hence, in this aspect, R₉ may be written as SO₃⁻Na⁺.

R₁₁, R₁₂, R₁₃ and R₁₄

For the options where any of R₁₁, R₁₂, R₁₃ and R₁₄ are each independently selected from ═CH₂, ═CH—(CH₂)ₛ—CH₃, ═CH—(CH₂)ₛ—R₂₅ and ═O, the carbon of the C-ring to which it is attached cannot have an optional double bond in order for the valence requirements of the molecule to be met. For example, if B is (B1) and R₁₁ is ═CH₂ and is positioned at the C1 position of the C-ring adjacent to the fused carbon of the C-ring, and R₁₂ and R₁₃ are each H then the resulting B group may be represented as:

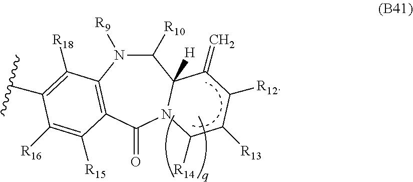

In the aspects where one of R₁₁ and R₁₂, R₁₂ and R₁₃, or R₁₃ and R₁₄ together with the carbon atoms to which they are attached form a 6-membered aryl, or a 5- or 6-membered cyclic, heterocyclic, or heteroaryl ring optionally substituted with up to three optional substituent groups, groups (B1)-(B3) contain a further fused ring (not drawn).

In these aspects, then the remaining groups (from R₁₁, R₁₂, R₁₃ and R₁₄) that do not form the further fused ring are each independently selected from the normal specified list of groups, i.e. from H, R₂₀, R₂₅, ═CH₂, ═CH—(CH₂)ₛ—CH₃, ═CH—(CH₂)ₛ—R₂₅, ═O, (CH₂)ₛ—OR₂₅, (CH₂)ₛ—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)ₛ—SO₂R₂₅, O—SO₂R₂₅, (CH₂)ₛ—C(O)R₂₅ and (CH₂)ₛ—C(O)NR₂₅R₂₆. For example, where B is (Bi), q is 1 and R₁₃ and R₁₄ together with the carbon atoms to which they are attached form a 6-membered aryl ring the structure of the group B can be shown as follows:

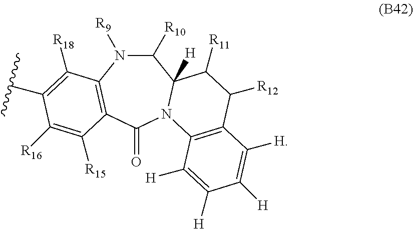

More suitably, in such aspects, B, (Bi) or (B4) is:

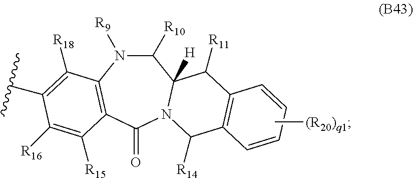

wherein q1 is 0, 1, 2 or 3.

Hence, there may be 0, 1, 2 or 3 optional independently selected R₂₀ groups present on the aromatic ring in (B43). More suitably q1 is 0 or 1.

In a more suitable aspect, B is (B1) and is (B10), (B11) or (B43).

Groups R₁₁ and R₁₂ do not form the further fused ring and so are each independently selected from the normal specified list of groups for R₁₁, R₁₂, R₁₃ and R₁₄. In addition, the H groups shown on the further fused ring of (B42) may be substituted with up to three independently selected optional R₂₀ groups.

Suitably, where one of R₁ and R₁₂, R₁₂ and R₁₃, or R₁₃ and R₁₄ together with the carbon atoms to which they are attached form an optionally substituted 5- or 6-membered heterocyclic or heteroaryl ring the heterocyclic or heteroaryl ring comprises one nitrogen atom.

Suitably, R₁₁, R₁₂, R₁₃ and R₁₄ are each independently selected from H, R₂₀, R₂₅, ═CH—(CH₂)ₛ—R₂₅, (CH₂)ₛ—OR₂₅, (CH₂)ₛ—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)ₛ—SO₂R₂₅, O—SO₂R₂₅, (CH₂)ₛ—C(O)R₂₅, (CH₂)ₛ—C(O)NR₂₅R₂₆;
or one of R₁ and R₁₂, R₁₂ and R₁₃, or R₁₃ and R₁₄ together with the carbon atoms to which they are attached form a 6-membered aryl, or a 5- or 6-membered cyclic, heterocyclic, or heteroaryl ring optionally substituted with up to three independently selected optional R₂₀ groups.

Suitably, R₁₁, R₁₂, R₁₃ and R₁₄ are each independently selected from H, R₂₀, R₂₅, (CH₂)ₛ—OR₂₅, (CH₂)ₛ—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)ₛ—C(O)R₂₅ and (CH₂)ₛ—C(O)NR₂₅R₂₆;
or one of R₁ and R₁₂, R₁₂ and R₁₃, or R₁₃ and R₁₄ together with the carbon atoms to which they are attached form a 6-membered aryl, or a 5- or 6-membered cyclic, heterocyclic, or heteroaryl ring optionally substituted with up to three independently selected optional R₂₀ groups.

Suitably at least one of R₁₁, R₁₂, R₁₃ and R₁₄ is H.

Suitably, R₁₁ is H.

Suitably, R₁₂ is H.

Suitably, R₁₃ is H.

Suitably, R₁₄ is H.

R₁₅,_R₁₆, R₁₇ and R₁₈

Suitably, R₁₅, R₁₆ R₁₇ and R₁₈ are each independently selected from H and R₂₀.

Suitably, R₁₅, R₁₆ R₁₇ and R₁₈ are each independently selected from H, (CH₂)ⱼ—OH, methyl, ethyl, OCH₃, OCH₂CH₃, OCH₂Ph, CO₂H, CO₂CH₃, CO₂CH₂CH₃, O—(CH₂)ₜ—NH₂ and (CH₂)ₛ—NH₂.

More suitably, R₁₅, R₁₆ R₁₇ and R₁₈ are each independently selected from H, (CH₂)ⱼ—OH, OCH₃, OCH₂CH₃, OCH₂Ph and (CH₂)ₛ—NH₂.

More suitably, R₁₅ is H.

More suitably, R₁₆ is OCH₃.

More suitably, R₁₇ is OCH₃.

More suitably, R₁₈ is H.

R₁₉, R₂₁, R₂₂, R₂₃, R₂₄, R₂₆, R₂₇ and R₂₈

Suitably each R₁₉, R₂₁, R₂₂, R₂₃, R₂₄, R₂₆, R₂₇ and R₂₈ is independently selected from H, methyl, ethyl, n-propyl, i-propyl, n-butyl, s-butyl, i-butyl and t-butyl.

Suitably each R₁₉, R₂₁, R₂₂, R₂₃, R₂₄, R₂₆, R₂₇ and R₂₈ is independently selected from H, methyl, and ethyl. More suitably each R₁₉, R₂₁, R₂₂, R₂₃, R₂₄, R₂₆, R₂₇ and R₂₈ is independently selected from H and methyl.

R₂₀

Suitably, each R₂₀ is independently selected from (CH₂)ⱼ—OH, methyl, ethyl, OCH₃, OCH₂CH₃, OCH₂Ph, (CH₂)ⱼ—CO₂R₂₇, O—(CH₂)ₖ—NR₂₇R₂₈, (CH₂)ⱼ—NR₂₇R₂₈, C(═O)—NH—(CH₂)ₖ—NR₂₇R₂₈, C(═O)—NH—C₆H₄—(CH₂)ⱼ—R₂₇ and C(═O)—NH—(CH₂)ₖ—C(═NH)NR₂₇R₂₈.

Suitably, each R₂₀ is independently selected from (CH₂)ⱼ—OH, methyl, ethyl, OCH₃, OCH₂CH₃, OCH₂Ph, (CH₂)ⱼ—CO₂H, (CH₂)ⱼ—CO₂CH₃, (CH₂)ⱼ—CO₂CH₂CH₃, O—(CH₂)ₖ—NH₂, O—(CH₂)ₖ—NH—CH₃, (CH₂)ⱼ—NH₂, (CH₂)ⱼ—NH—CH₃, C(═O)—NH—(CH₂)ₖ—NH₂, C(═O)—NH—(CH₂)ₖ—NH—CH₃, C(═O)—NH—C₆H₄—(CH₂)ⱼ—H, C(═O)—NH—(CH₂)ₖ—C(═NH)NH₂ and C(═O)—NH—(CH₂)ₖ—C(═NH)NH—CH₃.

More suitably, each R₂₀ is independently selected from (CH₂)ⱼ—OH, methyl, ethyl, OCH₃, OCH₂CH₃, CO₂H, CO₂CH₃, CO₂CH₂CH₃, O—(CH₂)ₖ—NH₂ and (CH₂)ⱼ—NH₂.

Suitably, one R₂₀ group is selected from O—(CH₂)ₖ—NR₂₇R₂₈, (CH₂)ⱼ—NR₂₇R₂₈, C(═O)—NH—(CH₂)ₖ—NR₂₇R₂₈; C(═O)—NH—C₆H₄—(CH₂)ⱼ—R₂₇ and C(═O)—NH—(CH₂)ₖ—C(═NH)NR₂₇R₂₈; and the remaining R₂₀ groups are each independently selected from (CH₂)ⱼ—OH, C₁₋₆ alkyl, OC₁₋₆ alkyl, OCH₂Ph and (CH₂)ⱼ—CO₂R₂₇.

More suitably, one R₂₀ group is selected from O—(CH₂)ₖ—NH₂, O—(CH₂)ₖ—NH—CH₃, (CH₂)ⱼ—NH₂, (CH₂)ⱼ—NH—CH₃, C(═O)—NH—(CH₂)ₖ—NH₂, C(═O)—NH—(CH₂)ₖ—NH—CH₃, C(═O)—NH—C₆H₄—(CH₂)ⱼ—H, C(═O)—NH—(CH₂)ₖ—C(═NH)NH₂ and C(═O)—NH—(CH₂)ₖ—C(═NH)NH—CH₃; and the remaining R₂₀ groups are each independently selected from (CH₂)ⱼ—OH, methyl, ethyl, OCH₃, OCH₂CH₃, OCH₂Ph, (CH₂)ⱼ—CO₂H, (CH₂)ⱼ—CO₂CH₃ and (CH₂)ⱼ—CO₂CH₂CH₃.

More suitably, one R₂₀ group is selected from O—(CH₂)ₖ—NH₂ and (CH₂)ⱼ—NH₂; and the remaining R₂₀ groups are each independently selected from (CH₂)ⱼ—OH, methyl, ethyl, OCH₃, OCH₂CH₃, CO₂H, CO₂CH₃, CO₂CH₂CH₃.

R₂₅

Suitably R₂₅ is selected from C₅₋₉ heteroaryl, C₆₋₁₅ heteroarylalkyl, phenyl, benzyl and phenethyl; wherein the heteroaryl, heteroarylalkyl, phenyl and aralkyl groups are optionally substituted with up to three independently selected optional R₂₀ groups.

Suitably R₂₅ is selected from H, C₁₋₁₂ alkyl, N-methylpyrrolyl, furanyl, thiophenyl, N-methylimidazolyl, oxazolyl, thiazolyl, pyridyl, indolyl, N-methylindolyl, benzofuranyl, benzothiophenyl, benzimidazolyl, N-methylbenzoimidazolyl, benzooxazolyl, benzothiazolyl, pyrrol-3-ylmethyl, pyrrol-4-ylmethyl, imidazol-2-ylmethyl, imidazol-4-ylmethyl, thiophen-3-ylmethyl, furan-3-ylmethyl, phenyl, benzyl and phenethyl; wherein the heteroaryl, heteroarylalkyl, phenyl and aralkyl groups are optionally substituted with up to three independently selected optional R₂₀ groups.

Suitably R₂₅ is selected from H, C₁₋₆ alkyl, N-methylpyrrolyl, furanyl, thiophenyl, N-methylimidazolyl, oxazolyl, thiazolyl, pyridyl, indolyl, N-methylindolyl, benzofuranyl, benzothiophenyl, benzimidazolyl, N-methylbenzoimidazolyl, benzooxazolyl, benzothiazolyl, pyrrol-3-ylmethyl, pyrrol-4-ylmethyl, imidazol-2-ylmethyl, imidazol-4-ylmethyl, thiophen-3-ylmethyl, furan-3-ylmethyl, phenyl, benzyl and phenethyl; wherein the heteroaryl, heteroarylalkyl, phenyl and aralkyl groups are optionally substituted with up to three independently selected optional R₂₀ groups.

Suitably R₂₅ is selected from H, methyl, ethyl, n-propyl, i-propyl, n-butyl, s-butyl, i-butyl, t-butyl, N-methylpyrrolyl, furanyl, thiophenyl, N-methylimidazolyl, oxazolyl, thiazolyl, pyridyl, indolyl, N-methylindolyl, benzofuranyl, benzothiophenyl, benzimidazolyl, N-methylbenzoimidazolyl, benzooxazolyl, benzothiazolyl, pyrrol-3-ylmethyl, pyrrol-4-ylmethyl, imidazol-2-ylmethyl, imidazol-4-ylmethyl, thiophen-3-ylmethyl, furan-3-ylmethyl, phenyl, benzyl and phenethyl optionally substituted with up to three independently selected optional R₂₀ groups.

Suitably R₂₅ is selected from H, methyl, ethyl, n-propyl, i-propyl, n-butyl, s-butyl, i-butyl, t-butyl, N-methylpyrrolyl, furanyl, thiophenyl, N-methylimidazolyl, oxazolyl, thiazolyl, pyridyl, indolyl, N-methylindolyl, benzofuranyl, benzothiophenyl, benzimidazolyl, N-methylbenzoimidazolyl, benzooxazolyl, benzothiazolyl, phenyl, benzyl and phenethyl optionally substituted with up to three independently selected optional R₂₀ groups.

In some embodiments, R₂₅ is selected from H, methyl, ethyl, n-propyl, i-propyl, n-butyl, s-butyl, i-butyl, t-butyl.

R₂₉, R₃₀, R₃₁ and R₃₂

R₂₉, R₃₀, R₃₁ and R₃₂ are each independently selected from H and R₂₀.

Suitably, R₂₉, R₃₀, R₃₁ and R₃₂ are each independently selected from H, (CH₂)ⱼ—OH, methyl, ethyl, OCH₃, OCH₂CH₃, OCH₂Ph, CO₂H, CO₂CH₃, CO₂CH₂CH₃, O—(CH₂)ₜ—NH₂ and (CH₂)ₛ—NH₂.

More suitably, R₂₉, R₃₀, R₃₁ and R₃₂ are each independently selected from H, (CH₂)ⱼ—OH, OCH₃, OCH₂CH₃, OCH₂Ph and (CH₂)ₛ—NH₂.

More suitably, R₂₉ is H.

More suitably, R₃₀ is H.

More suitably, R₃₁ is H.

More suitably, R₃₂ is H.

In some aspects, one of R₂₉, R₃₀, R₃₁ and R₃₂ is selected from O—(CH₂)ₖ—NR₂₇R₂₈, (CH₂)ⱼ—NR₂₇R₂₈, C(═O)—NH—(CH₂)ₖ—NR₂₇R₂₈; C(═O)—NH—C₆H₄—(CH₂)ⱼ—R₂₇ and C(═O)—NH—(CH₂)ₖ—C(═NH)NR₂₇R₂₈; and the remaining of R₂₉, R₃₀, R₃₁ and R₃₂ are each independently selected from H, OH, C₁₋₆ alkyl, OC₁₋₆ alkyl, OCH₂Ph and (CH₂)ⱼ—CO₂R₂₇.

In some aspects, one of R₂₉, R₃₀, R₃₁ and R₃₂ is selected from O—(CH₂)_{g}—NR₂₆R₂₇, (CH₂)_{f}—NR₂₆R₂₇, C(═O)—NH—(CH₂)_{g}—NR₂₆R₂₇, C(═O)—NH—C₆H₄—(CH₂)_{f}—R₂₀ and C(═O)—NH—(CH₂)_{g}—C(═NH)NR₂₆R₂₇; and the remaining of R₂₉, R₃₀, R₃₁ and R₃₂ are H.

R₃₃, R₃₄ and R₃₅

R₃₃, R₃₄ and R₃₅ are each independently selected from H and R₂₀.

Suitably, R₃₃, R₃₄ and R₃₅ are each independently selected from H, (CH₂)ⱼ—OH, methyl, ethyl, OCH₃, OCH₂CH₃, OCH₂Ph, CO₂H, CO₂CH₃, CO₂CH₂CH₃, O—(CH₂)ₜ—NH₂ and (CH₂)—NH₂.

More suitably, R₃₃, R₃₄ and R₃₅ are each independently selected from H, (CH₂)ⱼ—OH, OCH₃, OCH₂CH₃, OCH₂Ph and (CH₂)ₛ—NH₂.

More suitably, R₃₃ is H.

More suitably, R₃₄ is H.

More suitably, R₃₅ is H.

In some aspects, one of R₃₃, R₃₄ and R₃₅ is selected from O—(CH₂)ₖ—NR₂₇R₂₈, (CH₂)ⱼ—NR₂₇R₂₈, C(═O)—NH—(CH₂)ₖ—NR₂₇R₂₈; C(═O)—NH—C₆H₄—(CH)ⱼ—R₂₇ and C(═O)—NH—(CH₂)ₖ—C(═NH)NR₂₇R₂₈; and the remaining of R₃₃, R₃₄ and R₃₅ are each independently selected from H, (CH₂)ⱼ—OH, C₁₋₆ alkyl, OC₁₋₆ alkyl, OCH₂Ph and (CH₂)ⱼ—CO₂R₂₇.

In some aspects, one of R₃₃, R₃₄ and R₃₅ is selected from O—(CH₂)_{g}—NR₂₆R₂₇, (CH₂)_{f}—NR₂₆R₂₇, C(═O)—NH—(CH₂)_{g}—NR₂₆R₂₇, C(═O)—NH—C₆H₄—(CH₂)_{f}—R₂₀ and C(═O)—NH—(CH₂)_{g}—C(═NH)NR₂₆R₂₇; and the remaining of R₃₃, R₃₄ and R₃₅ are H.

Combinations

Suitably, when Y⁶ is —(CH₂)_{z}— at least one of R₄, R₅, R₆, R₇, R₈, R₉, R₁₁, R₁₂, R₁₃, R₁₄, R₁₅, R₁₆, R₁₇ and R₁₈ is selected from H, C₁₋₆ alkyl, OC₁₋₆ alkyl and OCH₂Ph; suitably, at least 30 two, three, four, five, six, seven, eight, nine, ten or eleven of R₄, R₅, R₆, R₇, R₈, R₉, R₁₁, R₁₂, R₁₃, R₁₄, R₁₅, R₁₆, R₁₇ and R₁₈ are selected from H, C₁₋₆ alkyl, OC₁₋₆ alkyl and OCH₂Ph.

Suitably, when Y⁶ is —(CH₂)_{z}— at least one of R₄, R₅, R₆, R₇, R₈, R₉, R₁₁, R₁₂, R₁₃, R₁₄, R₁₅, R₁₆, R₁₇ and R₁₈ is H; suitably, at least two, three, four, five, six, seven, eight, nine, ten or eleven of R₅, R₆, R₈, R₉, R₁₁, R₁₂, R₁₃, R₁₆ and R₁₇ are H.

In some aspects, suitably, when Y⁶ is —(CH₂)_{z}— one of R₄, R₅, R₆, R₇, R₈, R₉, R₁₁, R₁₂, R₁₃, R₁₄, R₁₅, R₁₆, R₁₇ and R₁₈ is selected from OH, (CH₂)ⱼ—CO₂R₂₇, O—(CH₂)ₖ—NR₂₇R₂₈, (CH₂)ⱼ—NR₂₇R₂₈, C(═O)—NH—(CH₂)ₖ—NR₂₇R₂₈, C(═O)—NH—C₆H₄—(CH₂)ⱼ—R₂₇ and C(═O)—NH—(CH₂)ₖ—C(═NH)NR₂₇R₂₈. Suitably the remaining of R₄, R₅, R₆, R₇, R₈, R₉, R₁₁, R₁₂, R₁₃, R₁₄, R₁₅, R₁₆, R₁₇ and R₁₈ are selected from H, C₁₋₆ alkyl, OC₁₋₆ alkyl and OCH₂Ph.

Suitably, when Y⁶ is (Li) at least one of R₄, R₅, R₆, R₇, R₈, R₉, R₁₁, R₁₂, R₁₃, R₁₄, R₁₅, R₁₆, R₁₇, R₁₈, R₂₉, R₃₀, R₃₁ and R₃₂ is selected from H, C₁₋₆ alkyl, OC₁₋₆ alkyl and OCH₂Ph; suitably, at least two, three, five, six, seven, eight, nine, ten, eleven, twelve, thirteen or fourteen of R₄, R₅, R₆, R₇, R₈, R₉, R₁₁, R₁₂, R₁₃, R₁₄, R₁₅, R₁₆, R₁₇, R₁₈, R₂₉, R₃₀, R₃₁ and R₃₂ are selected from H, C₁₋₆ alkyl, OC₁₋₆ alkyl and OCH₂Ph.

Suitably, when Y⁶ is (Li) at least one of R₄, R₅, R₆, R₇, R₈, R₉, R₁₁, R₁₂, R₁₃, R₁₄, R₁₅, R₁₆, R₁₇, R₁₈, R₂₉, R₃₀, R₃₁ and R₃₂ is H; suitably, at least two, three, five, six, seven, eight, nine, ten, eleven, twelve, thirteen or fourteen of R₄, R₅, R₆, R₇, R₈, R₉, R₁₁, R₁₂, R₁₃, R₁₄, R₁₅, R₁₆, R₁₇, R₁₈, R₂₉, R₃₀, R₃₁ and R₃₂ are H.

In some aspects, suitably, when Y⁶ is (L1) one R₄, R₅, R₆, R₇, R₈, R₉, R₁₁, R₁₂, R₁₃, R₁₄, R₁₅, R₁₆, R₁₇, R₁₈, R₂₉, R₃₀, R₃₁ and R₃₂ is selected from OH, (CH₂)ⱼ—CO₂R₂₇, O—(CH₂)ₖ—NR₂₇R₂₈, (CH₂)ⱼ—NR₂₇R₂₈, C(═O)—NH—(CH₂)ₖ—NR₂₇R₂₈, C(═O)—NH—C₆H₄—(CH₂)ⱼ—R₂₇ and C(═O)—NH—(CH₂)ₖ—C(═NH)NR₂₇R₂₈. Suitably the remaining of R₄, R₅, R₆, R₇, R₈, R₉, R₁₁, R₁₂, R₁₃, R₁₄, R₁₅, R₁₆, R₁₇, R₁₈, R₂₉, R₃₀, R₃₁ and R₃₂ are selected from H, C₁₋₆ alkyl, OC₁₋₆ alkyl and OCH₂Ph.

In some aspects, the compound of formula (I) and salts, solvates and tautomers thereof are selected with the proviso that Y⁶ is a group (Li) when A, B, p and q are selected as (A1), (B1), 1 and 0 respectively.

In some aspects, the compound of formula (I) and salts, solvates and tautomers thereof are selected with the proviso that Y⁶ is a group (L1) when A is selected from (A1), (A2) and (A3); and B, h, p and q are (B1), 0, 1 and 0 respectively. Suitably in this aspect, Y⁶ is selected from monocyclic heteroarylene, monocyclic cycloalkylene, monocyclic cycloalkenylene and monocyclic heterocyclylene groups optionally substituted with up to three independently selected optional R₂₀ groups.

R^{A}

Suitably, each R^{A} is independently selected from -het- and -X^{A}-T²—X^{A}-.

R^{B}

Suitably, each R^{B} is independently selected from H and C₁₋₈ alkyl. More suitably, each R^{B} is independently selected from H and C₁₋₆ alkyl. More suitably, each R^{B} is independently selected from H, methyl, ethyl, propyl and butyl.

R^{C}

Suitably, each R^{C} is independently selected from H and C₁₋₈ alkyl. More suitably, each R^{C} is independently selected from H and C₁₋₆ alkyl. More suitably, each R^{C} is independently selected from H, methyl, ethyl, propyl and butyl.

T¹

Suitably, each T¹ is selected from —C(O), —C(O)(CH₂)₀₋₂₀C(O)—, —C(O)PhC(O)—. Suitably, each T¹ is selected from —C(O), —C(O)(CH₂)₀₋₁₀C(O)—, —C(O)PhC(O)—. Suitably, each T¹ is selected from —C(O), —C(O)(CH₂)₀₋₅C(O)—, —C(O)PhC(O)—. Suitably, each T¹ is selected from —C(O), —C(O)C(O)—, —C(O)(CH₂)C(O)—, —C(O)(CH₂)₂C(O)—, —C(O)(CH₂)₃C(O)—, —C(O)(CH₂)₄C(O)—, —C(O)PhC(O)—.

X^{A}

Suitably, each X^{A} is independently selected from a bond, —NH—, —N(C₁₋₈ alkyl)- and —O—.

het

Suitably, het is a mono-, bi-, or tricyclic heteroarylene of 5 to 10 members, suitably, 5 to 9 members.

Suitably, het is a mono-, bi-, or tricyclic heteroarylene containing one or two, heteroatoms independently selected from O, N, S, P and B.

Suitably, het is a mono- or bicyclic heteroarylene of 5 to 12 members.

Suitably, mono-, bi-, or tricyclic heteroarylene containing one, two, or there heteroatoms independently selected from O, N and S.

Suitably het is substituted with up to three independently selected optional R₂₀ groups.

f

Suitably, each f is an integer independently selected from 0 to 40; suitably independently selected from 0 to 30; suitably, from 0 to 20; suitably, from 0 to 10; suitably, from 0 to 9; suitably, from 0 to 8; suitably, from 0 to 7; suitably, from 0 to 6; suitably, from 0 to 5; suitably, from 0 to 4; suitably, from 0 to 3; suitably, from 0 to 2; suitably, from 0 to 1.

g

Suitably, each g is an integer independently selected from 0 to 40; suitably independently selected from 0 to 30; suitably, from 0 to 20; suitably, from 0 to 10; suitably, from 0 to 9; suitably, from 0 to 8; suitably, from 0 to 7; suitably, from 0 to 6; suitably, from 0 to 5; suitably, from 0 to 4; suitably, from 0 to 3; suitably, from 0 to 2; suitably, from 0 to 1.

h

In some aspects, h is 1. In other aspects, h is 0. Suitably, h is 0.

i

Each j is an integer independently selected from 0 to 6; that is each j is independently selected from 0, 1, 2, 3, 4, 5 and 6.

Suitably, each j is an integer independently selected from 0 to 5; suitably independently selected from 0 to 4; suitably independently selected from 0 to 3; suitably independently selected from 0 to 2; suitably independently selected from 0 to 1.

In some aspects, j is 0.

k

Each k is an integer independently selected from 1 to 6; that is each k is independently selected from 1, 2, 3, 4, 5 and 6.

Suitably, each k is an integer independently selected from 1 to 5; suitably independently selected from 1 to 4; suitably independently selected from 1 to 3; suitably independently selected from 1 to 2.

In some aspects, k is 1.

m

m is an integer selected from 0 to 12; that is m is selected from 0, 1, 2, 3, 4, 5, 6, 7, 8, 9, 10, 11 and 12.

Suitably, m is an integer selected from 0 to 11; suitably selected from 0 to 10; suitably selected from 0 to 9; suitably selected from 0 to 8; suitably selected from 0 to 7; suitably selected from 0 to 6; suitably selected from 0 to 5; suitably selected from 0 to 4; suitably selected from 0 to 3; suitably selected from 0 to 2; suitably selected from 0 to 1.

In some aspects, m is 0.

n

n is an integer selected from 0 to 12; that is n is selected from 0, 1, 2, 3, 4, 5, 6, 7, 8, 9, 10, 11 and 12.

Suitably, n is an integer selected from 0 to 11; suitably selected from 0 to 10; suitably selected from 0 to 9; suitably selected from 0 to 8; suitably selected from 0 to 7; suitably selected from 0 to 6; suitably selected from 0 to 5; suitably selected from 0 to 4; suitably selected from 0 to 3; suitably selected from 0 to 2; suitably selected from 0 to 1.

In some aspects, n is 1.

p

In some aspects, p is 1. In other aspects, p is 0. Suitably, p is 0.

q

In some aspects, q is 1. In other aspects, q is 0. Suitably, q is 1.

s

Each s is an integer independently selected from 0 to 6; that is each s is independently selected from 0, 1, 2, 3, 4, 5 and 6.

Suitably, each s is an integer independently selected from 0 to 5; suitably independently selected from 0 to 4; suitably independently selected from 0 to 3; suitably independently selected from 0 to 2; suitably independently selected from 0 to 1.

In some aspects, s is 0.

t

Each t is an integer independently selected from 1 to 6; that is each t is independently selected from 1, 2, 3, 4, 5 and 6.

Suitably, each t is an integer independently selected from 1 to 5; suitably independently selected from 1 to 4; suitably independently selected from 1 to 3; suitably independently selected from 1 to 2.

In some aspects, t is 1.

w

Suitably, each w is an integer independently selected from 1 to 40; suitably independently selected from 1 to 30; suitably, from 1 to 20; suitably, from 1 to 10; suitably, from 1 to 9; suitably, from 1 to 8; suitably, from 1 to 7; suitably, from 1 to 6; suitably, from 1 to 5; suitably, from 1 to 4; suitably, from 1 to 3; suitably, from 1 to 2. Suitably, w is 1.

z

Each z is an integer selected from 1 to 5; that is z is selected from 1, 2, 3, 4 and 5.

Suitably, z is an integer selected from 1 to 4; suitably selected from 1 to 3; suitably selected from 1 to 2.

In some aspects, z is 1.

Prodrug Moiety R′″

A prodrug moiety is a masked form of an active drug that needs to be transformed before exhibiting its pharmacological action. Typically, such moieties are designed to be activated after an enzymatic or chemical reaction once they have been administered into the body. Activation of prodrugs typically involves the elimination of the prodrug moiety to release the drug. Prodrugs are considered to be inactive or at least significantly less active than the released drugs.

Several prodrug moieties are known for group A, such as CPI or CBI groups, in compounds of formula (I). In particular, prodrug moieties containing carbonyl, carbamoyl, glycosyl, O-amino, O-acylamino, para-aminobenzyl ether, peptidyl or phosphate groups have been reported in Wolff, I., et al, Clin. Cancer Res. 1996, 2, 1717-1723; Wang, Y., et al, Bioorg. Med. Chem. 2006, 14, 7854-7861; Tietze, L. F., et al, J. Med. Chem. 2009, 52, 537-543; Jin, W., et al, J. Am. Chem. Soc. 2007, 129, 15391-15397; Jeffrey, et al, J. Med. Chem. 2005, 48, 1344-1358; Boger, D. L., et al, Synthesis 1999, 1505-1509; Tercel, M., et al, J. Org. Chem. 1999, 64, 5946-5953; Nagamura, S., et al, Bioorg. Med. Chem. 1997, 5, 623-630; and Zhao, R. Y. et al, J. Med. Chem. 2011, 55, 766-782.

Suitably, the prodrug moiety R′″ is selected from —O—NHR₁₉, —O—NR₁₉Boc, P(O)(OH)₂, —O—NHSO₂R₁₉, —O—C(═O)—NR′R″, —O—NHC(O)C(CH₃)₃, —O—NHCO₂R₁₉, —NHCONH₂, —O—

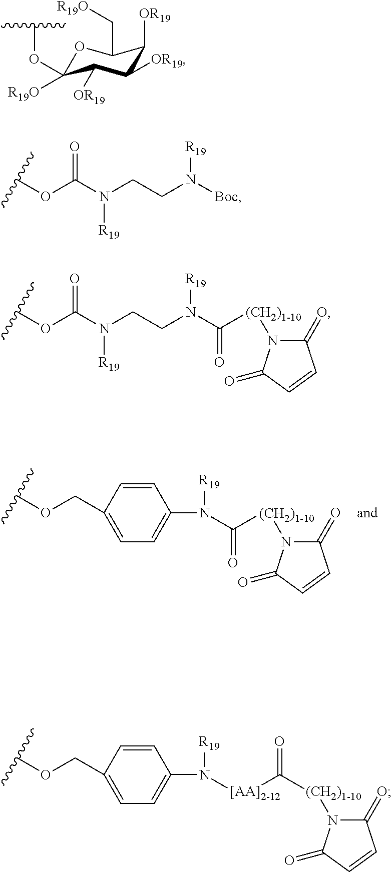

wherein R′ and R″ together with the nitrogen to which they are attached form a 5- or 6-membered heterocyclic ring optionally substituted with 1, 2 or 3 C₁₋₆ alkyl groups; and wherein each AA is an independently selected amino acid.

Hence, the —(CH₂)₁₋₁₀— linker consists of 1, 2, 3, 4, 5, 6, 7, 8, 9 or 10 CH₂ units. Suitably, such linkers consist of 3, 4, 5, 6 or 7 CH₂ units.

Hence, the -[AA]₂₋₁₂- is a peptide group consisting of 2, 3, 4, 5, 6, 7, 8, 9, 10, 11 or 12 amino acid units. Suitably, this peptide group consist of 2, 3, 4, 5, 6, 7 or 8 amino acid units.

More suitably, the prodrug moiety R′″ is selected from —O—NH₂, —O—NHCH₃, —O—P(O)(OH)₂, —O—NHBoc, —O—NCH₃Boc, —O—NHSO₂CH₃,

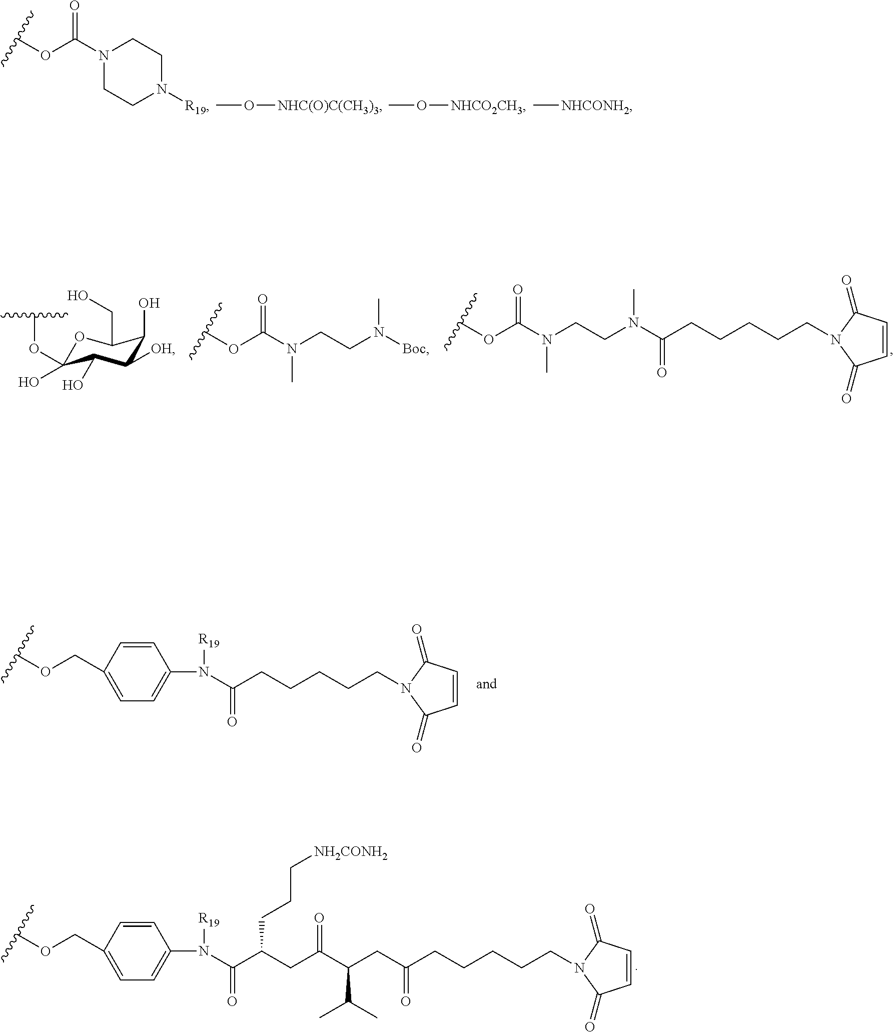

More suitably, the prodrug moiety is:

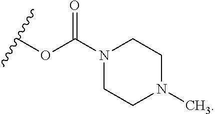 [SMILES: *OC(=O)N1CCN(C)CC1]

R′ and R″

Suitably, R′ and R″ together with the nitrogen to which they are attached form a 6-membered heterocyclic ring optionally substituted with 1, 2 or 3 C₁₋₆ alkyl groups.

More suitably, R′ and R″ together with the nitrogen to which they are attached form:

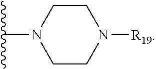

More suitably, R′ and R″ together with the nitrogen to which they are attached form:

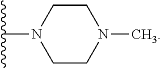 [SMILES: *N1CCN(C)CC1]

Other Aspects

In some aspects, the compound of formula (I) is selected with the proviso that when the compound is:

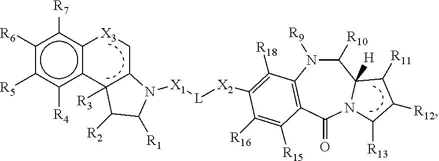

at least one of R₁₁, R₁₂ and R₁₃ is independently selected from C₅₋₉ heteroaryl, C₆₋₁₅ heteroarylalkyl, phenyl and C₇₋₁₂ aralkyl groups and these groups are optionally substituted with up to three independently selected optional R₂₀ groups. In such aspect, the remain groups of R₁₁, R₁₂ and R₁₃ that are not selected from C₅₋₉ heteroaryl, C₆₋₁₅ heteroarylalkyl, phenyl and C₇₋₁₂ aralkyl groups, are selected from the normal specified list of substituents, i.e. they are independently selected from H, R₂₀, R₂₅, ═CH₂, ═CH—(CH₂)ₛ—CH₃, ═CH—(CH₂)ₛ—R₂₅, ═O, (CH₂)ₛ—OR₂₅, (CH₂)ₛ—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)ₛ—SO₂R₂₅, O—SO₂R₂₅, (CH₂)ₛ—C(O)R₂₅ and (CH₂)ₛ—C(O)NR₂₅R₂₆; or one of R₁₁ and R₁₂, R₁₂ and R₁₃, or R₁₃ and R₁₄ together with the carbon atoms to which they are attached form a 6-membered aryl, or a 5- or 6-membered cyclic, heterocyclic, or heteroaryl ring optionally substituted with up to three independently selected optional R₂₀ groups.

In some aspects, the compound of formula (I) is selected with the proviso that when the compound is:

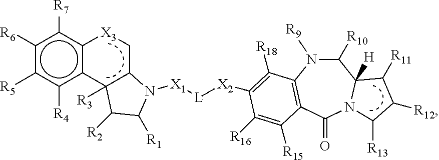

that one of R₁₁ and R₁₂ or R₁₂ and R₁₃, or R₁₃ together with the carbon atoms to which they are attached form a 6-membered aryl, or a 5- or 6-membered cyclic, heterocyclic, or heteroaryl ring optionally substituted with up to three independently selected optional R₂₀ groups. In such aspects, the PBD moiety comprises a further fused ring and the remaining group out of R₁₁, R₁₂ and R₁₃ that does not form part of this further fused ring is selected from the normal specified list of substituents, i.e. from H, R₂₀, R₂₅, ═CH₂, ═CH—(CH₂)ₛ—CH₃, ═CH—(CH₂)ₛ—R₂₅, ═O, (CH₂)ₛ—OR₂₅, (CH₂)ₛ—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)ₛ—SO₂R₂₅, O—SO₂R₂₅, (CH₂)ₛ—C(O)R₂₅ and (CH₂)ₛ—C(O)NR₂₅R₂₆.

In some aspects, the compound of formula (I) is selected with the proviso that R₅ and R₆ are each independently selected from H and R₂₀ when B, q and A are selected as (B1), 0 and (A4) respectively, hence, in these aspects when the compound of formula (I) has the following structure:

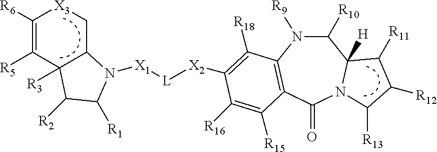

that R₅ and R₆ are each independently selected from H and R₂₀.

Suitably, the compounds of formula (I) and salts, solvates and tautomers thereof are selected with the proviso that at least one of R₁₁, R₁₂ and R₁₃ is independently selected from C₅₋₉ heteroaryl, C₆₋₁₅ heteroarylalkyl, phenyl and C₇₋₁₂ aralkyl groups and these groups are optionally substituted with up to three independently selected optional R₂₀ groups when B, q, A, p and h are selected as (B1), 0, (A1), 1 and 0 respectively; and with the proviso that R₅ and R₆ are each independently selected from H and R₂₀ when B, q and A are selected as (B1), 0 and (A4) respectively.

Suitably, the compounds of formula (I) and salts, solvates and tautomers thereof are selected with the proviso that either p is 0 or h is 1 when B, q and A are selected as (B1), 0, (A1), 1 and 0 respectively; and with the proviso that R₅ and R₆ are each independently selected from H and R₂₀ when B, q and A are selected as (B1), 0 and (A4) respectively.

Suitably, the compounds of formula (I) and salts, solvates and tautomers thereof are selected with the proviso that A is selected from (A2), (A3), (A4) and (A5) when B, q and A are selected as (B1), 0, (A1), 1 and 0 respectively; and with the proviso that R₅ and R₆ are each independently selected from H and R₂₀ when B, q and A are selected as (B1), 0 and (A4) respectively.

In some aspects, the compound of formula (I) is selected with the proviso that when R₂ is C₁₋₆ alkyl that R₉ and R₁₀ are selected from options (i), (ii), (iii) or (iv). When R₂ is C₁₋₆ alkyl then the moiety A of the compound of formula (I) will not alkylate DNA. In such aspects, the options for R₉ and R₁₀ are limited to those that ensure that the moiety B of the compound of formula (I) does alkylate with DNA. Examples of compounds that fall within this proviso are:

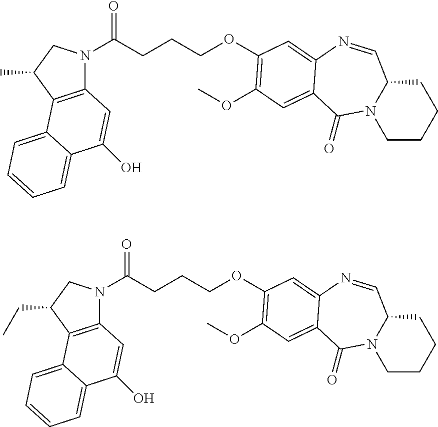 [SMILES: COc1cc2c(cc1OCCCC(=O)N1C[C@@H](C)c3c1cc(O)c1ccccc31)N=C[C@@H]1CCCCN1C2=O ; CC[C@@H]1CN(C(=O)CCCOc2cc3c(cc2OC)C(=O)N2CCCC[C@H]2C=N3)c2cc(O)c3ccccc3c21]

In some aspects, the compound of formula (I) is selected with the proviso that when (v) R₉ is H or C₁₋₆ alkyl, and R₁₀ is oxo or H; then either R₂ is selected from —CH₂-halogen and H, and R₃ is H; or R₂ and R₃ together with the carbon atoms to which they are attached form a cyclopropyl ring. When option (v) applies then the moiety B of the compound of formula (I) will not alkylate DNA. In such aspects, the options for R₂ are limited to those that ensure that the moiety A of the compound of formula (I) does alkylate with DNA. Examples of compounds that fall within this proviso are shown below:

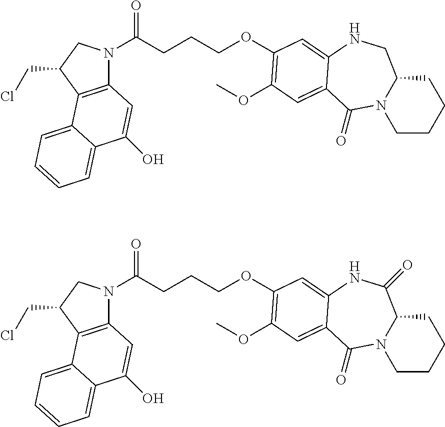 [SMILES: COc1cc2c(cc1OCCCC(=O)N1C[C@@H](CCl)c3c1cc(O)c1ccccc31)NC[C@@H]1CCCCN1C2=O ; COc1cc2c(cc1OCCCC(=O)N1C[C@@H](CCl)c3c1cc(O)c1ccccc31)NC(=O)[C@@H]1CCCCN1C2=O]

In the top compound R is H, R₁₀ is H; and R₂ is —CH₂—Cl. In the bottom compound, R) is H, R₁₀ is oxo and R₂ is —CH—Cl.

Applications

In some aspects, the present invention relates to a compound of formula (I) and salts, solvates and tautomers thereof, for use as a drug in an antibody-drug conjugate. Suitably, a compound of formula (I) and salts, solvates and tautomers thereof, for use as a drug in an antibody-drug conjugate by attaching to an antibody or an antibody fragment via an optional linker group. Suitably, the compound of formula (I) and salts, solvates and tautomers thereof, is attached to an antibody or an antibody fragment via a linker group. Suitably, the antibody-drug conjugate is for use in for treatment of a disease, more specifically of a proliferative disease.

In some aspects, the present invention relates to the use of a compound of formula (I) and salts, solvates and tautomers thereof, as a drug in an antibody-drug conjugate. Suitably, the use of a compound of formula (I) and salts, solvates and tautomers thereof, as a drug in an antibody-drug conjugate by attaching to an antibody or an antibody fragment via an optional linker group. Suitably, the compound of formula (I) and salts, solvates and tautomers thereof, is attached to an antibody or an antibody fragment via a linker group. Suitably, the antibody-drug conjugate is for use in for treatment of a disease, more specifically of a proliferative disease. Suitably, the drug may be attached by any suitable functional group that it contains to the antibody or antibody fragment optionally via a linker group. Typically, the drug contains one or more functional groups such as amine, hydroxyl or carboxylic acid groups for attaching the drug to the antibody or antibody fragment optionally via a linker group.

The invention finds application in the treatment of disease, more specifically of a proliferative disease.

The term “proliferative disease” refers to an unwanted or uncontrolled cellular proliferation of excessive or abnormal cells which is undesired, such as, neoplastic or hyperplastic growth, whether in vitro or in vivo. Examples of proliferative conditions include, but are not limited to, benign, pre-malignant, and malignant cellular proliferation, including but not limited to, neoplasms and tumours (e.g. histocytoma, glioma, astrocyoma, osteoma), cancers (e.g. lung cancer, small cell lung cancer, hepatocellular cancer, gastric or stomach cancer including gastrointestinal cancer, bowel cancer, colon cancer, hepatoma, breast cancer, glioblastoma, cervical cancer, ovarian cancer, oesophageal [or esophageal] cancer, oral cancer, prostate cancer, testicular cancer, liver cancer, rectal cancer, colorectal cancer, endometrial or uterine carcinoma, uterine cancer, salivary gland carcinoma, kidney or renal cancer, prostate cancer, vulval cancer, thyroid cancer, hepatic carcinoma, anal carcinoma, penile carcinoma, head and neck cancer, bladder cancer, pancreas cancer, brain cancer, sarcoma, osteosarcoma, Kaposi's sarcoma, melanoma), leukemias, psoriasis, bone diseases, fibroproliferative disorders (e.g. of connective tissues), and atherosclerosis. Suitably the proliferative disease is selected from bladder cancer, bone cancer, bowel cancer, brain cancer, breast cancer, cervical cancer, colon cancer, head and neck cancer, leukemia, liver cancer, lung cancer, lymphoma, melanoma, oesophageal cancer, oral cancer, ovarian cancer, pancreatic cancer, prostate cancer, rectal cancer, renal cancer, retinoblastoma, sarcoma, skin cancer, stomach cancer, testicular cancer, thyroid cancer and uterine cancer. Suitably the proliferative disease is selected from breast cancer and cervical cancer.

Any type of cell may be treated, including but not limited to, bone, eye, head and neck, lung, gastrointestinal (including, e.g. mouth, oesophagus, bowel, colon), breast (mammary), cervix, ovarian, uterus, prostate, liver (hepatic), kidney (renal), bladder, pancreas, brain, and skin.

A skilled person is readily able to determine whether or not a candidate compound treats a proliferative condition for any particular cell type.

Suitably subjects are human, livestock animals and companion animals.

The compounds of formula (I) find application as payloads for antibodies or antibody fragments or other targeting moieties (e.g. hormones, proteins and small molecule targeting agents such as folic acid). The compounds of formula (I) readily allow conjugation to antibodies or antibody fragments or other targeting moieties.

The substituent groups of the compounds of formula (I) may interact with DNA sequences and may be selected so as to target specific sequences.

Antibody and Antibody Fragments

The term “antibody” specifically covers monoclonal antibodies, polyclonal antibodies, dimers, multimers, multispecific antibodies (e.g., bispecific antibodies), intact antibodies and antibody fragments, so long as they exhibit the desired biological activity, for example, the ability to bind CD19 (Miller et al (2003) Journal, of Immunology 170:4854-4861). Antibodies may be murine, human, humanized, chimeric, or derived from other species. An antibody is a protein generated by the immune system that is capable of recognizing and binding to a specific antigen. (Janeway, C, Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. lgG1, lgG2, lgG3, lgG4, lgA1 and lgA2) or subclass, or allotype (e.g. human G1 m1, G1 m2, G1 m3, non-G1 m1 [that, is any allotype other than G1 m1], G1 m17, G2m23, G3m21, G3m28, G3m1, G3m5, G3m13, G3m14, G3m10, G3m15, G3m16, G3m6, G3m24, G3m26, G3m27, A2m1, A2m2, Km1, Km2 and Km3) of immunoglobulin molecule. The immunoglobulins can be derived from any species, including human, murine, or rabbit origin.

As used herein, “binds an epitope” is used to mean the antibody binds an epitope with a higher affinity than a non-specific partner such as Bovine Serum Albumin (BSA, Genbank accession no. CAA76847, version no. CAA76847.1 Gl:3336842, record update date: Jan. 7, 2011 02:30 PM). In some embodiments the antibody binds an epitope with an association constant (Ka) at least 2, 3, 4, 5, 10, 20, 50, 100, 200, 500, 1000, 2000, 5000, 104, 10⁵ or 10⁶-fold higher than the antibody's association constant for BSA, when measured at physiological conditions.

“Antibody fragments” comprise a portion of a full length antibody, generally the antigen binding or variable region thereof. Examples of antibody fragments include Fab, Fab′, F(ab′)2, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments. The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences. An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1 q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., lgG1, lgG2, lgG3, lgG4, IgA, and lgA2. The heavy-chain constant domains that correspond to the different classes of antibodies are called α, δ, ε, γ, and μ, respectively. The subunit structures and three-dimensional configurations of different classes of immunoglobulins are well known.

The antibodies disclosed herein may be modified. For example, to make them less immunogenic to a human subject. This may be achieved using any of a number of techniques familiar to the person skilled in the art, such as humanisation.

Antibody-Drug Conjugates

Antibody therapy has been established for the targeted treatment of patients with cancer, immunological and angiogenic disorders (Carter, P. (2006) Nature Reviews Immunology 6:343-357). The use of antibody-drug conjugates (ADC), i.e. immunoconjugates, for the local delivery of cytotoxic or cytostatic agents, i.e. drugs to kill or inhibit tumor cells in the treatment of cancer, targets delivery of the drug moiety to tumors, and intracellular accumulation therein, whereas systemic administration of these unconjugated drug agents may result in unacceptable levels of toxicity to normal cells (Xie et al (2006) Expert. Opin. Biol. Ther. 6(3):281-291; Kovtun ef a/ (2006) Cancer Res. 66(6):3214-3121; Law et al (2006) Cancer Res. 66(4):2328-2337; Wu et al (2005) Nature Biotech. 23(9): 1137-1145; Lambert J. (2005) Current Opin. in Pharmacol. 5:543-549; Hamann P. (2005) Expert Opin. Ther. Patents 15(9): 1087-1 103; Payne, G. (2003) Cancer Cell 3:207-212; Trail ef a/ (2003) Cancer Immunol. Immunother. 52:328-337; Syrigos and Epenetos (1999) Anticancer Research 19:605-614).

Maximal efficacy with minimal toxicity is sought thereby. Efforts to design and refine ADC have focused on the selectivity of monoclonal antibodies (mAbs) as well as drug mechanism of action, drug-linking, drug/antibody ratio (loading), and drug-releasing properties (Junutula, et al., 2008b Nature Biotech., 26(8):925-932; Doman ef a/(2009) Blood 114(13):2721-2729; U.S. Pat. Nos. 7,521,541; 7,723,485; WO2009/052249; McDonagh (2006) Protein Eng. Design & Sel. 19(7): 299-307; Doronina ef a/ (2006) Bioconj. Chem. 17:114-124; Erickson ef a/ (2006) Cancer Res. 66(8): 1-8; Sanderson et a/ (2005) Clin. Cancer Res. 11:843-852; Jeffrey et al (2005) J. Med. Chem. 48:1344-1358; Hamblett et al (2004) Clin. Cancer Res. 10:7063-7070). Drug moieties may impart their cytotoxic and cytostatic effects by mechanisms including tubulin binding, DNA binding, proteasome and/or topoisomerase inhibition. Some cytotoxic drugs tend to be inactive or less active when conjugated to large antibodies or protein receptor ligands.

Tumor-Associated Antigens:

(1) BMPR1B (bone morphogenetic protein receptor-type IB, Genbank accession no. NM_001203)

ten Dijke, P., et al Science 264 (5155): 101-104 (1994), Oncogene 14 (11): 1377-1382 (1997); WO2004063362 (Claim 2); WO2003042661 (Claim 12); US2003134790-A1 (Page 38-39); WO2002102235 (Claim 13; Page 296); WO2003055443 (Page 91-92); WO200299122 (Example 2; Page 528-530); WO2003029421 (Claim 6); WO2003024392 (Claim 2; FIG. 112); WO200298358 (Claim 1; Page 183); WO200254940 (Page 100-101); WO200259377 (Page 349-350); WO200230268 (Claim 27; Page 376); WO200148204 (Example; FIG. 4) NP_001194 bone morphogenetic protein receptor, type IB/pid=NP_001194.1—Cross-references: MIM:603248; NP_001194.1; AY065994

(2) E16 (LAT1, SLC7A5, Genbank accession no. NM_003486)

Biochem. Biophys. Res. Commun. 255 (2), 283-288 (1999), Nature 395 (6699):288-291 (1998), Gaugitsch, H. W., et al (1992) J. Biol. Chem. 267 (16): 11267-11273); WO2004048938 (Example 2); WO2004032842 (Example TV); WO2003042661 (Claim 12); WO2003016475 (Claim 1); WO200278524 (Example 2); WO200299074 (Claim 19; Page 127-129); WO200286443 (Claim 27; Pages 222, 393); WO2003003906 (Claim 1o; Page 293); WO200264798 (Claim 33; Page 93-95); WO200014228 (Claim 5; Page 133-136); US2003224454 (FIG. 3); WO2003025138 (Claim 12; Page 150); NP_003477 solute carrier family 7 (cationic amino acid transporter, y+system), member 5/pid=NP_003477.3 —Homo sapiens; Cross-references: MIM:600182; NP_003477.3; NM_015923; NM_o03486_1

(3) STEAP1 (six transmembrane epithelial antigen of prostate, Genbank accession no. NM_012449)

Cancer Res. 61 (15), 5857-5860 (2001), Hubert, R. S., et al (1999) Proc. Natl. Acad. Sci. U.S.A. 96 (25): 14523-14528); WO2004065577 (Claim 6); WO2004027049 (FIG. 1L); EP1394274 (Example 11); WO2004016225 (Claim 2); WO2003042661 (Claim 12); US2003157089 (Example 5); US2003185830 (Example 5); US2003064397 (FIG. 2); WO200289747 (Example 5; Page 618-619); WO2003022995 (Example 9; FIG. 13A, Example 53; Page 173, Example 2; FIG. 2A); NP_036581 six transmembrane epithelial antigen of the prostate; Cross-references: MIM:604415; NP_036581.1; NM_012449_1

(4) 0772P (CA125, MUC16, Genbank accession no. AF361486)

J. Biol. Chem. 276 (29):27371-27375 (2001)); WO2004045553 (Claim 14); WO200292836 (Claim 6; FIG. 12); WO200283866 (Claim 15; Page 116-121); US2003124140 (Example 16); US 798959; Cross-references: GI:34501467; AAK74120.3; AF361486_1

(5) MPF (MPF, MSLN, SMR, megakaryocyte potentiating factor, mesothelin, Genbank accession no. NM_005823) Yamaguchi, N., et al Biol. Chem. 269 (2), 805-808 (1994), Proc. Natl. Acad. Sci. U.S.A. 96 (20): 11531-11536 (1999), Proc. Nat. Acad. Sci. U.S.A. 93 (1): 136-140 (1996), J. Biol. Chem. 270 (37):21984-21990 (1995)); WO2003101283 (Claim 14); (WO2002102235 (Claim 13; Page 287-288); WO2002101075 (Claim 4; Page 308-309); WO200271928 (Page 320-321); WO9410312 (Page 52-57); Cross-references: MIM:601051; NP_005814.2; NM_005823_1

(6) Napi2b (Napi3b, NAPI-3B, NPTIIb, SLC34A2, solute carrier family 34 (sodium phosphate), member 2, type II sodium-dependent phosphate transporter 3b, Genbank accession no. NM_006424) J. Biol. Chem. 277 (22): 19665-19672 (2002), Genomics 62 (2):281-284 (1999), Feild, J. A., et al (1999) Biochem. Biophys. Res. Commun. 258 (3):578-582); WO2004022778 (Claim 2); EP1394274 (Example 11); WO2002102235 (Claim 13; Page 326); EP875569 (Claim 1; Page 17-19); WO200157188 (Claim 20; Page 329); WO2004032842 (Example IV); WO200175177 (Claim 24; Page 139-140); Cross-references: MIM:604217; NP_006415.1; NM_006424_1

(7) Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B, Genbank accession no. AB040878) Nagase T., et al (2000) DNA Res. 7 (2): 143-150); WO2004000997 (Claim 1); WO2003003984 (Claim 1); WO200206339 (Claim 1; Page 50); WO200188133 (Claim 1; Page 41-43, 48-58); WO2003054152 (Claim 20); WO2003101400 (Claim 11); Accession: Q9P283; EMBL; AB040878; BAA95969.1. Genew; HGNC: 10737;

(8) PSCA hlg (2700050C12Rik, C5300080O16Rik, RIKEN cDNA 2700050C12, RIKEN cDNA 2700050C12 gene, Genbank accession no. AY358628); Ross et al (2002) Cancer Res. 62:2546-2553; US2003129192 (Claim 2); US2004044180 (Claim 12); US2004044179 (Claim 11); US2003096961 (Claim 11); US2003232056 (Example 5); WO2003105758 (Claim 12); US2003206918 (Example 5); EP1347046 (Claim 1); WO2003025148 (Claim 20); Cross-references: GI:37182378; AAQ88991.1; AY358628_1

(9) ETBR (Endothelin type B receptor, Genbank accession no. AY275463); Nakamuta M., et al Biochem. Biophys. Res. Commun. 177, 34-39, 1991; Ogawa Y., et al Biochem. Biophys. Res. Commun. 178, 248-255, 1991; Arai H., et al Jpn. Circ. J. 56, 1303-1307, 1992; Arai H., et al J. Biol. Chem. 268, 3463-3470, 1993; Sakamoto A., Yanagisawa M., et al Biochem. Biophys. Res. Commun. 178, 656-663, 1991; Elshourbagy N. A., et al J. Biol. Chem. 268, 3873-3879, 1993; Haendler B., et al J. Cardiovasc. Pharmacol. 20, S1-S4, 1992; Tsutsumi M., et al Gene 228, 43-49, 1999; Strausberg R. L., et al Proc. Natl. Acad. Sci. U.S.A. 99, 16899-16903, 2002; Bourgeois C, et al J. Clin. Endocrinol. Metab. 82, 3116-3123, 1997; Okamoto Y., et al Biol. Chem. 272, 21589-21596, 1997; Verheij J. B., et al Am. J. Med. Genet. 108, 223-225, 2002; Hofstra R. M. W., et al Eur. J. Hum. Genet. 5, 180-185, 1997; Puffenberger E. G., et al Cell 79, 1257-1266, 1994; Attie T., et al, Hum. Mol. Genet. 4, 2407-2409, 1995; Auricchio A., et al Hum. Mol. Genet. 5:351-354, 1996; Amiel J., et al Hum. Mol. Genet. 5, 355-357, 1996; Hofstra R. M. W., et al Nat. Genet. 12, 445-447, 1996; Svensson P J., et al Hum. Genet. 103, 145-148, 1998; Fuchs S., et al Mol. Med. 7,115-124, 2001; Pingault V., et al (2002) Hum. Genet. 111, 198-206; WO2004045516 (Claim 1); WO2004048938 (Example 2); WO2004040000 (Claim 151); WO2003087768 (Claim 1); WO2003016475 (Claim 1); WO2003016475 (Claim 1); WO200261087 (FIG. 1); WO2003016494 (FIG. 6); WO2003025138 (Claim 12; Page 144); WO200198351 (Claim 1; Page 124-125); EP522868 (Claim 8; FIG. 2); WO200177172 (Claim 1; Page 297-299); US2003109676; U.S. Pat. No. 6,518,404 (FIG. 3); U.S. Pat. No. 5,773,223 (Claim 1a; Col 31-34); WO2004001004;

(10) MSG783 (RNF124, hypothetical protein FLJ20315, Genbank accession no. NM_017763); WO2003104275 (Claim 1); WO2004046342 (Example 2); WO2003042661 (Claim 12); WO2003083074 (Claim 14; Page 61); WO2003018621 (Claim 1); WO2003024392 (Claim 2; FIG. 93); WO200166689 (Example 6); Cross-references: LocusID: 54894; NP_060233.2; NM_017763_1

(11) STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein, Genbank accession no. AF455138)

Lab. Invest. 82 (11): 1573-1582 (2002); WO2003087306; US2003064397 (Claim 1; FIG. 1); WO200272596 (Claim 13; Page 54-55); WO200172962 (Claim 1; FIG. 4B); WO2003104270 (Claim 11); WO2003104270 (Claim 16); US2004005598 (Claim 22); WO2003042661 (Claim 12); US2003060612 (Claim 12; FIG. 10); WO200226822 (Claim 23; FIG. 2); WO200216429 (Claim 12; FIG. 10); Cross-references: GI:22655488; AAN04080.1; AF455138_1

(12) TrpM4 (BR22450, FLJ20041, TRPM4, TRPM4B, transient receptor potential cation channel, subfamily M, member 4, Genbank accession no. NM_017636) Xu, X. Z., et al Proc. Natl. Acad. Sci. U.S.A. 98 (19): 10692-10697 (2001), Cell 109 (3):397-407 (2002), J. Biol. Chem. 278 (33):30813-30820 (2003); US2003143557 (Claim 4); WO200040614 (Claim 14; Page 100-103); WO200210382 (Claim 1; FIG. 9A); WO2003042661 (Claim 12); WO200230268 (Claim 27; Page 391); US2003219806 (Claim 4); WO200162794 (Claim 14; FIG. 1A-D); Cross-references: MIM:606936; NP_060106.2; NM_017636_1

(13) CRIPTO (CR, CR1, CRGF, CRIPTO, TDGF1, teratocarcinoma-derived growth factor, Genbank accession no. NP_003203 or NM_003212)

Ciccodicola, A., et al EMBO J. 8 (7): 1987-1991 (1989), Am. J. Hum. Genet. 49 (3):555-565 (1991); US2003224411 (Claim 1); WO2003083041 (Example 1); WO2003034984 (Claim 12); WO200288170 (Claim 2; Page 52-53); WO2003024392 (Claim 2; FIG. 58); WO200216413 (Claim 1; Page 94-95, 105); WO200222808 (Claim 2; FIG. 1); U.S. Pat. No. 5,854,399 (Example 2; Col 17-18); U.S. Pat. No. 5,792,616 (FIG. 2); Cross-references: MIM: 187395; NP_003203.1; NM_003212_1

(14) CD21 (CR2 (Complement receptor 2) or C3DR (C3d/Epstein Barr virus receptor) or Hs.73792 Genbank accession no. M26004)

Fujisaku et al (1989) J. Biol. Chem. 264 (4):2118-2125); Weis J. J., et al J. Exp. Med. 167, 1047-1066, 1988; Moore M., et al Proc. Natl. Acad. Sci. U.S.A. 84, 9194-9198, 1987; Barel M., et al Mol. Immunol. 35, 1025-1031, 1998; Weis J. J., et al Proc. Natl. Acad. Sci. U.S.A. 83, 5639-5643, 1986; Sinha S. K., et al (1993) J. Immunol. 150, 5311-5320; WO2004045520 (Example 4); US2004005538 (Example 1); WO2003062401 (Claim 9); WO2004045520 (Example 4); WO9102536 (FIGS. 9.1-9.9); WO2004020595 (Claim 1); Accession: P20023; Q13866; Q14212; EMBL; M26004; AAA35786.1.

(15) CD79b (CD79B, CD79R, IGb (immunoglobulin-associated beta), B29, Genbank accession no. NM_000626 or 11038674)

Proc. Natl. Acad. Sci. U.S.A. (2003) 100 (7):4126-4131, Blood (2002) 100 (9):3068-3076, Muller et al (1992) Eur. J. Immunol. 22 (6): 1621-1625); WO2004016225 (claim 2, FIG. 140); WO2003087768, US2004101874 (claim 1, page 102); WO2003062401 (claim 9); WO200278524 (Example 2); US2002150573 (claim 5, page 15); U.S. Pat. No. 5,644,033; WO2003048202 (claim 1, pages 306 and 309); WO 99/558658, U.S. Pat. No. 6,534,482 (claim 13, FIG. 17A/B); WO200055351 (claim 11, pages 1145-1146); Cross-references: MIM: 147245; NP_000617.1; NM_000626_1

(16) FcRH2 (IFGP4, IRTA4, SPAP1A (SH2 domain containing phosphatase anchor protein 1a), SPAP1B, SPAP1C, Genbank accession no. NM_030764, AY358130) Genome Res. 13 (10):2265-2270 (2003), Immunogenetics 54 (2):87-95 (2002), Blood 99 (8):2662-2669 (2002), Proc. Natl. Acad. Sci. U.S.A. 98 (17):9772-9777 (2001), Xu, M. J., et al (2001) Biochem. Biophys. Res. Commun. 280 (3):768-775; WO2004016225 (Claim 2); WO2003077836; WO200138490 (Claim 5; FIG. 18D-1-18D-2); WO2003097803 (Claim 12); WO2003089624 (Claim 25); Cross-references: MIM:606509; NP_110391.2; NM_030764_1

(17) HER2 (ErbB2, Genbank accession no. M11730)

Coussens L., et al Science (1985) 230(4730): 1132-1139); Yamamoto T., et al Nature 319, 230-234, 1986; Semba K., et al Proc. Natl. Acad. Sci. U.S.A. 82, 6497-6501, 1985; Swiercz J. M., et al J. Cell Biol. 165, 869-880, 2004; Kuhns J. J., et al J. Biol. Chem. 274, 36422-36427, 1999; Cho H.-S., et al Nature 421, 756-760, 2003; Ehsani A, et al (1993) Genomics 15, 426-429; WO2004048938 (Example 2); WO2004027049 (FIG. 11); WO2004009622; WO2003081210; WO2003089904 (Claim 9); WO2003016475 (Claim 1); US2003118592; WO2003008537 (Claim 1); WO2003055439 (Claim 29; FIG. 1 A-B); WO2003025228 (Claim 37; FIG. 5C); WO200222636 (Example 13; Page 95-107); WO200212341 (Claim 68; FIG. 7); WO200213847 (Page 71-74); WO200214503 (Page 114-117); WO200153463 (Claim 2; Page 41-46); WO200141787 (Page 15); WO200044899 (Claim 52; FIG. 7); WO200020579 (Claim 3; FIG. 2); U.S. Pat. No. 5,869,445 (Claim 3; Col 31-38); WO9630514 (Claim 2; Page 56-61); EP1439393 (Claim 7); WO2004043361 (Claim 7); WO2004022709; WO200100244 (Example 3; FIG. 4); Accession: P04626; EMBL; M11767; AAA35808.1. EMBL; M11761; AAA35808.1.

(18) NCA (CEACAM6, Genbank accession no. M18728);

Barnett T., et al Genomics 3, 59-66, 1988; Tawaragi Y., et al Biochem. Biophys. Res. Commun. 150, 89-96, 1988; Strausberg R. L., et al Proc. Natl. Acad. Sci. U.S.A. 99: 16899-16903, 2002; WO2004063709; EP 1439393 (Claim 7); WO2004044178 (Example 4); WO2004031238; WO2003042661 (Claim 12); WO200278524 (Example 2); WO200286443 (Claim 27; Page 427); WO200260317 (Claim 2); Accession: P40199; Q14920; EMBL; M29541; AAA59915.1. EMBL; M18728;

(19) MDP (DPEP1, Genbank accession no. BCo17023)

Proc. Natl. Acad. Sci. U.S.A. 99 (26): 16899-16903 (2002); WO2003016475 (Claim 1); WO200264798 (Claim 33; Page 85-87); JP05003790 (FIG. 6-8); WO9946284 (FIG. 9); Cross-references: MIM: 179780; AAH17023.1; BCo17023_1

(20) IL20Rα (IL20Ra, ZCYTOR7, Genbank accession no. AF 184971); Clark H. F., et al Genome Res. 13, 2265-2270, 2003; Mungall A. J., et al Nature 425, 805-811, 2003; Blumberg H., et al Cell 104, 9-19, 2001; Dumoutier L., et al J. Immunol. 167, 3545-3549, 2001; Parrish-Novak J., et al J. Biol. Chem. 277, 47517-47523, 2002; Pletnev S., et al (2003) Biochemistry 42: 12617-12624; Sheikh F., et al (2004) J. Immunol. 172, 2006-2010; EP1394274 (Example 11); US2004005320 (Example 5); WO2003029262 (Page 74-75); WO2003002717 (Claim 2; Page 63); WO200222153 (Page 45-47); US2002042366 (Page 20-21); WO200146261 (Page 57-59); WO200146232 (Page 63-65); W09837193 (Claim 1; Page 55-59); Accession: Q9UHF4; Q6UWA9; Q96SH8; EMBL; AF 184971; AAF01320.1.

(21) Brevican (BCAN, BEHAB, Genbank accession no. AF229053)

Gary S. C., et al Gene 256, 139-147, 2000; Clark H. F., et al Genome Res. 13, 2265-2270, 2003; Strausberg R. L., et al Proc. Natl. Acad. Sci. U.S.A. 99, 16899-16903, 2002; US2003186372 (Claim 11); US2003186373 (Claim 11); US2003119131 (Claim 1; FIG. 52); US2003119122 (Claim 1; FIG. 52); US2003119126 (Claim 1); US2003119121 (Claim 1; FIG. 52); US2003119129 (Claim 1); US2003119130 (Claim 1); US2003119128 (Claim 1; FIG. 52); US2003119125 (Claim 1); WO2003016475 (Claim 1); WO200202634 (Claim 1);

(22) EphB2R (DRT, ERK, Hek5, EPHT3, Tyr05, Genbank accession no. NM_004442) Chan, J. and Watt, V. M., Oncogene 6 (6), 1057-1061 (1991) Oncogene 10 (5):897-905 (1995), Annu. Rev. Neurosci. 21:309-345 (1998), Int. Rev. Cytol. 196: 177-244 (2000); WO2003042661 (Claim 12); WO200053216 (Claim 1; Page 41); WO2004065576 (Claim 1); WO2004020583 (Claim 9); WO2003004529 (Page 128-132); WO200053216 (Claim 1; Page 42); Cross-references: MIM: 600997; NP_004433.2; NM_004442_1

(23) ASLG659 (B7h, Genbank accession no. AX092328)

US20040101899 (Claim 2); WO2003104399 (Claim 11); WO2004000221 (FIG. 3); US2003165504 (Claim 1); US2003124140 (Example 2); US2003065143 (FIG. 60); WO2002102235 (Claim 13; Page 299); US2003091580 (Example 2); WO200210187 (Claim 6; FIG. 10); WO200194641 (Claim 12; FIG. 7b); WO200202624 (Claim 13; FIG. 1A-1B); US2002034749 (Claim 54; Page 45-46); WO200206317 (Example 2; Page 320-321, Claim 34; Page 321-322); WO200271928 (Page 468-469); WO200202587 (Example 1; FIG. 1); WO200140269 (Example 3; Pages 190-192); WO200036107 (Example 2; Page 205-207); WO2004053079 (Claim 12); WO2003004989 (Claim 1); WO200271928 (Page 233-234, 452-453); WO 0116318;

(24) PSCA (Prostate stem cell antigen precursor, Genbank accession no. AJ297436) Reiter R. E., et al Proc. Nat. Acad. Sci. U.S.A. 95, 1735-1740, 1998; Gu Z., et al Oncogene 19, 1288-1296, 2000; Biochem. Biophys. Res. Commun. (2000) 275(3):783-788; WO2004022709; EP1394274 (Example 11); US2004018553 (Claim 17); WO2003008537 (Claim 1); WO200281646 (Claim 1; Page 164); WO2003003906 (Claim 10; Page 288); WO200140309 (Example 1; FIG. 17); US2001055751 (Example 1; FIG. 1b); WO200032752 (Claim 18; FIG. 1); WO9851805 (Claim 17; Page 97); WO9851824 (Claim 10; Page 94); WO9840403 (Claim 2; FIG. 1B); Accession: 043653; EMBL; AF043498; AAC39607.1.

(25) GEDA (Genbank accession No. AY260763);

AAP14954 lipoma HMGIC fusion-partner-like protein/pid=AAP14954.1 —Homo sapiens Species: Homo sapiens (human)

WO2003054152 (Claim 20); WO200300842 (Claim 1); WO2003023013 (Example 3, Claim 20); US2003194704 (Claim 45); Cross-references: GI:30102449; AAP14954.1; AY260763_1

(26) BAFF-R (B cell-activating factor receptor, BLyS receptor 3, BR3, Genbank accession No. AF116456); BAFF receptor/pid=NP_443177.1 —Homo sapiens Thompson, J. S., et al Science 293 (5537), 2108-2111 (2001); WO2004058309; WO2004011611; WO2003045422 (Example; Page 32-33); WO2003014294 (Claim 35; FIG. 6B); WO2003035846 (Claim 70; Page 615-616); WO200294852 (Col 136-137); WO200238766 (Claim 3; Page 133); WO200224909 (Example 3; FIG. 3); Cross-references: MIM:606269; NP_443177.1; NM_052945_1; AF132600

(27) CD22 (B-cell receptor CD22-B isoform, BL-CAM, Lyb-8, Lyb8, SIGLEC-2, FLJ22814, Genbank accession No. AK026467);

Wilson et al (1991) J. Exp. Med. 173: 137-146; WO2003072036 (Claim 1; FIG. 1); Cross-references: MIM: 107266; NP_001762.1; NM_001771_1

(28) CD79a (CD79A, CD79a, immunoglobulin-associated alpha, a B cell-specific protein that covalently interacts with Ig beta (CD79B) and forms a complex on the surface with Ig M molecules, transduces a signal involved in B-cell differentiation), pI: 4.84, MW: 25028 TM: 2 [P] Gene Chromosome: 19q13.2, Genbank accession No. NP_001774.10)

WO2003088808, US20030228319; WO2003062401 (claim 9); US2002150573 (claim 4, pages 13-14); WO9958658 (claim 13, FIG. 16); WO9207574 (FIG. 1); U.S. Pat. No. 5,644,033; Ha et al (1992) J. Immunol. 148(5): 1526-1531; Mueller et al (1992) Eur. J. Biochem. 22: 1621-1625; Hashimoto et al (1994) Immunogenetics 40(4):287-295; Preud'homme et al (1992) Clin. Exp. Immunol. 90(1): 141-146; Yu et al (1992) J. Immunol. 148(2) 633-637; Sakaguchi et al (1988) EMBO J. 7(11):3457-3464;

(29) CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia); 372 aa, pI: 8.54 MW: 41959 TM: 7 [P] Gene Chromosome: 1 1q23.3, Genbank accession No. NP_001707.1)

WO2004040000; WO2004015426; US2003105292 (Example 2); U.S. Pat. No. 6,555,339 (Example 2); WO200261087 (FIG. 1); WO200157188 (Claim 20, page 269); WO200172830 (pages 12-13); WO200022129 (Example 1, pages 152-153, Example 2, pages 254-256); WO9928468 (claim 1, page 38); U.S. Pat. No. 5,440,021 (Example 2, col 49-52); WO9428931 (pages 56-58); WO9217497 (claim 7, FIG. 5); Dobner et al (1992) Eur. J. Immunol. 22:2795-2799; Barella et al (1995) Biochem. J. 309:773-779;

(30) HLA-DOB (Beta subunit of MHC class II molecule (la antigen) that binds peptides and presents them to CD4+T lymphocytes); 273 aa, pI: 6.56 MW: 30820 TM: 1 [P] Gene Chromosome: 6p21.3, Genbank accession No. NP_002111.1) Tonnelle et al (1985) EMBO J. 4(11):2839-2847; Jonsson et al (1989) Immunogenetics 29(6):411-413; Beck et al (1992) J. Mol. Biol. 228:433-441; Strausberg et al (2002) Proc. Natl. Acad. Sci USA 99: 16899-16903; Servenius et al (1987) J. Biol. Chem. 262:8759-8766; Beck et al (1996) J. Mol. Biol. 255: 1-13; Naruse et al (2002) Tissue Antigens 59:512-519; WO9958658 (claim 13, FIG. 15); U.S. Pat. No. 6,153,408 (Col 35-38); U.S. Pat. No. 5,976,551 (col 168-170); US6011146 (col 145-146); Kasahara et al (1989) Immunogenetics 30(1):66-68; Larhammar et al (1985) J. Biol. Chem. 260(26): 14111-14119;

(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), pI: 7.63, MW: 47206 TM: 1 [P] Gene Chromosome: 17p13.3, Genbank accession No. NP_002552.2)

Le et al (1997) FEBS Lett. 418(1-2): 195-199; WO2004047749; WO2003072035 (claim 10); Touchman et al (2000) Genome Res. 10: 165-173; WO200222660 (claim 20); WO2003093444 (claim 1); WO2003087768 (claim 1); WO2003029277 (page 82);

(32) CD72 (B-cell differentiation antigen CD72, Lyb-2) PROTEIN SEQUENCE Full maeaity . . . tafrfpd (1..359; 359 aa), pI: 8.66, MW: 40225 TM: 1 [P] Gene Chromosome: 9p13.3, Genbank accession No. NP_001773.1)

WO2004042346 (claim 65); WO2003026493 (pages 51-52, 57-58); WO200075655 (pages 105-106); Von Hoegen et al (1990) J. Immunol. 144(12):4870-4877; Strausberg et al (2002) Proc. Natl. Acad. Sci USA 99: 16899-16903;

(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosis); 661 aa, pI: 6.20, MW: 74147 TM: 1 [P] Gene Chromosome: 5412, Genbank accession No. NP_005573.1) US2002193567; WO9707198 (claim 11, pages 39-42); Miura et al (1996) Genomics 38(3):299-304; Miura et al (1998) Blood 92:2815-2822; WO2003083047; WO9744452 (claim 8, pages 57-61); WO200012130 (pages 24-26);

(34) FcRH1 (Fc receptor-like protein 1, a putative receptor for the immunoglobulin Fc domain that contains C2 type Ig-like and ITAM domains, may have a role in B-lymphocyte differentiation); 429 aa, pI: 5.28, MW: 46925 TM: 1 [P] Gene Chromosome: 1q21-1q22, Genbank accession No. NP_443170.1) WO2003077836; WO200138490 (claim 6, FIG. 18E-1-18-E-2); Davis et al (2001) Proc. Natl. Acad. Sci USA 98(17):9772-9777; WO2003089624 (claim 8); EP1347046 (claim 1); WO2003089624 (claim 7);

(35) IRTA2 (Immunoglobulin superfamily receptor translocation associated 2, a putative immunoreceptor with possible roles in B cell development and lymphomagenesis; deregulation of the gene by translocation occurs in some B cell malignancies); 977 aa, pI: 6.88 MW: 106468 TM: 1 [P] Gene Chromosome: 1q21, Genbank accession No. Human: AF343662, AF343663, AF343664, AF343665, AF369794, AF397453, AK090423, AK090475, AL834187, AY358085; Mouse: AK089756, AY158090, AY506558; NP_112571.1 WO2003024392 (claim 2, FIG. 97); Nakayama et al (2000) Biochem. Biophys. Res. Commun. 277(1): 124-127; WO2003077836; WO200138490 (claim 3, FIG. 18B-1-18B-2);

(36) TENB2 (TMEFF2, tomoregulin, TPEF, HPP1, TR, putative transmembrane proteoglycan, related to the EGF/heregulin family of growth factors and follistatin); 374 aa, NCBI Accession: AAD55776, AAF91397, AAG49451, NCBI RefSeq: NP_057276; NCBI Gene: 23671; OMIM: 605734; SwissProt Q9UIK5; Genbank accession No. AF179274; AY358907, CAF85723, CQ782436

WO2004074320 (SEQ ID NO 810); JP2004113151 (SEQ ID NOS 2, 4, 8); WO2003042661 (SEQ ID NO 580); WO2003009814 (SEQ ID NO 411); EP1295944 (pages 69-70); WO200230268 (page 329); WO200190304 (SEQ ID NO 2706); US2004249130; US2004022727; WO2004063355; US2004197325; US2003232350; US2004005563; US2003124579; Horie et al (2000) Genomics 67: 146-152; Uchida et al (1999) Biochem. Biophys. Res. Commun. 266:593-602; Liang et al (2000) Cancer Res. 60:4907-12; Glynne-Jones et al (2001) Int J Cancer. October 15; 94(2): 178-84;

(37) PMEL17 (silver homolog; SILV; D12S53E; PMEL17; SI; SIL); ME20; gp100) BC001414; BT007202; M32295; M77348; NM_006928; McGlinchey, R. P. et al (2009) Proc. Natl. Acad. Sci. U.S.A. 106 (33), 13731-13736; Kummer, M. P. et al (2009) J. Biol. Chem. 284 (4), 2296-2306;

(38) TMEFF1 (transmembrane protein with EGF-like and two follistatin-like domains 1; Tomoregulin-1); H7365; C9orf2; C90RF2; U19878; X83961; NM_080655; NM_003692; Harms, P. W. (2003) Genes Dev. 17 (21), 2624-2629; Gery, S. et al (2003) Oncogene 22 (18):2723-2727;

(39) GDNF-Ra1 (GDNF family receptor alpha 1; GFRA1; GDNFR; GDNFRA; RETL1; TRNR1; RET1L; GDNFR-alpha1; GFR-ALPHA-1); U95847; BC014962; NM_145793 NM_005264; Kim, M. H. et al (2009) Mol. Cell. Biol. 29 (8), 2264-2277; Treanor, J. J. et al (1996) Nature 382 (6586):80-83;

(40) Ly6E (lymphocyte antigen 6 complex, locus E, Ly67, RIG-E, SCA-2, TSA-1); NP_002337.1; NM_002346.2; de Nooij-van Dalen, A G. et al (2003) Int. J. Cancer 103 (6), 768-774; Zammit, D. J. et al (2002) Mol. Cell. Biol. 22 (3):946-952; WO 2013/17705;

(41) TMEM46 (shisa homolog 2 (Xenopus laevis); SHISA2); NP_001007539.1; NM_001007538.1; Furushima, K. et al (2007) Dev. Biol. 306 (2), 480-492; Clark, H. F. et al (2003) Genome Res. 13 (10):2265-2270;

(42) Ly6G6D (lymphocyte antigen 6 complex, locus G6D; Ly6-D, MEGT1); NP_067079.2; NM_021246.2; Mallya, M. et al (2002) Genomics 80 (1): 113-123; Ribas, G. et al (1999) J. Immunol. 163 (1):278-287;

(43) LGR5 (leucine-rich repeat-containing G protein-coupled receptor 5; GPR49, GPR67); NP_003658.1; NM_003667.2; Salanti, G. et al (2009) Am. J. Epidemiol. 170 (5):537-545; Yamamoto, Y. et al (2003) Hepatology 37 (3):528-533;

(44) RET (ret proto-oncogene; MEN2A; HSCR1; MEN2B; MTC1; PTC; CDHF12; Hs.168114; RET51; RET-ELE1); NP_066124.1; NM_020975.4; Tsukamoto, H. et al (2009) Cancer Sci. 100 (10): 1895-1901; Narita, N. et al (2009) Oncogene 28 (34):3058-3068;

(45) LY6K (lymphocyte antigen 6 complex, locus K; LY6K; HSJ001348; FLJ35226); NP_059997.3; NM_017527.3; Ishikawa, N. et al (2007) Cancer Res. 67 (24): 11601-11611; de Nooij-van Dalen, A G. et al (2003) Int. J. Cancer 103 (6):768-774;

(46) GPR19 (G protein-coupled receptor 19; Mm.4787); NP_006134.1; NM_006143.2; Montpetit, A. and Sinnett, D. (1999) Hum. Genet. 105 (1-2): 162-164; O'Dowd, B. F. et al (1996) FEBS Lett. 394 (3):325-329;

(47) GPR54 (KISS1 receptor; KISSIR; GPR54; HOT7T175; AXOR12); NP_115940.2; NM 032551.4; Navenot, J. M. et al (2009) Mol. Pharmacol. 75 (6): 1300-1306; Hata, K. et al (2009) Anticancer Res. 29 (2):617-623;

(48) ASPHD1 (aspartate beta-hydroxylase domain containing 1; LOC253982); NP_859069.2; NM_181718.3; Gerhard, D. S. et al (2004) Genome Res. 14 (10B):2121-2127;

(49) Tyrosinase (TYR; OCA1A; OCA1A; tyrosinase; SHEP3); NP_000363.1; NM_000372.4; Bishop, D. T. et al (2009) Nat. Genet. 41 (8):920-925; Nan, H. et al (2009) Int. J. Cancer 125 (4): 909-917;

(50) TMEM118 (ring finger protein, transmembrane 2; RNFT2; FLJ14627); NP_001103373.1; NM 001109903.1; Clark, H. F. et al (2003) Genome Res. 13 (10):2265-2270; Scherer, S. E. et al (2006) Nature 440 (7082):346-351

(51) GPR172A (G protein-coupled receptor 172A; GPCR41; FLJ11856; D15Ertd747e); NP_078807.1; NM_024531.3; Ericsson, T. A. et al (2003) Proc. Natl. Acad. Sci. U.S.A. 100 (11):6759-6764; Takeda, S. et al (2002) FEBS Lett. 520 (1-3):97-101.

(52) CD33, a member of the sialic acid binding, immunoglobulin-like lectin family, is a 67-kDa glycosylated transmembrane protein. CD33 is expressed on most myeloid and monocytic leukemia cells in addition to committed myelomonocytic and erythroid progenitor cells. It is not seen on the earliest pluripotent stem cells, mature granulocytes, lymphoid cells, or nonhematopoietic cells (Sabbath et al., (1985) J. Clin. Invest. 75:756-56; Andrews et al., (1986) Blood 68: 1030-5). CD33 contains two tyrosine residues on its cytoplasmic tail, each of which is followed by hydrophobic residues similar to the immunoreceptor tyrosine-based inhibitory motif (ITIM) seen in many inhibitory receptors.

(53) CLL-1 (CLEC12A, MICL, and DCAL2), encodes a member of the C-type lectin/C-type lectin-like domain (CTL/CTLD) superfamily. Members of this family share a common protein fold and have diverse functions, such as cell adhesion, cell-cell signalling, glycoprotein turnover, and roles in inflammation and immune response. The protein encoded by this gene is a negative regulator of granulocyte and monocyte function. Several alternatively spliced transcript variants of this gene have been described, but the full-length nature of some of these variants has not been determined. This gene is closely linked to other CTL/CTLD superfamily members in the natural killer gene complex region on chromosome 12p13 (Drickamer K (1999) Curr. Opin. Struct. Biol. 9 (5):585-90; van Rhenen A, et al., (2007) Blood 110 (7):2659-66; Chen C H, et al. (2006) Blood 107 (4): 1459-67; Marshall A S, et al. (2006) Eur. J. Immunol. 36 (8):2159-69; Bakker A B, et al (2005) Cancer Res. 64 (22):8443-50; Marshall A S, et al (2004) J. Biol. Chem. 279 (15): 14792-802). CLL-1 has been shown to be a type II transmembrane receptor comprising a single C-type lectin-like domain (which is not predicted to bind either calcium or sugar), a stalk region, a transmembrane domain and a short cytoplasmic tail containing an ITIM motif.

Anti-CD22 Antibodies

In certain embodiments, the anti-CD22 antibodies of an ADC comprises three light chain hypervariable regions (HVR-L1, HVR-L2 and HVR-L3) and three heavy chain hypervariable regions (HVR-H1, HVR-H2 and HVR-H3), according to U.S. Pat. No. 8,226,945:

| (SEQ ID NO: 1) | |
|---|---|
| HVR-L₁ | RSSQSIVHSVGNTFLE |
| (SEQ ID NO: 2) | |
| HVR-L₂ | KVSNRFS |
| (SEQ ID NO: 3) | |
| HVR-L₃ | FQGSQFPYT |
| (SEQ ID NO: 4) | |
| HVR-H₁ | GYEFSRSWMN |
| (SEQ ID NO: 5) | |
| HVR-H₂ | GRIYPGDGDTNYSGKFKG |
| (SEQ ID NO: 6) | |
| HVR-H₃ | DGSSWDWYFDV |

Anti-Lv6E Antibodies

In certain embodiments, an ADC comprises anti-Ly6E antibodies. Lymphocyte antigen 6 complex, locus E (Ly6E), also known as retinoic acid induced gene E (RIG-E) and stem cell antigen 2 (SCA-2). It is a GPI linked, 131 amino acid length, ˜8.4 kDa protein of unknown function with no known binding partners. It was initially identified as a transcript expressed in immature thymocyte, thymic medullary epithelial cells in mice (Mao, et al. (1996) Proc. Natl. Acad. Sci. U.S.A. 93:5910-5914). In some embodiments, the invention provides an immunoconjugate comprising an anti-Ly6E antibody described in PCT Publication No. WO 2013/177055.

In some embodiments, the invention provides an antibody-drug conjugate comprising an anti-Ly6E antibody comprising at least one, two, three, four, five, or six HVRs selected from (a) HVR-H1 comprising the amino acid sequence of SEQ ID NO: 12; (b) HVR-H2 comprising the amino acid sequence of SEQ ID NO: 13; (c) HVR-H3 comprising the amino acid sequence of SEQ ID NO: 14; (d) HVR-L1 comprising the amino acid sequence of SEQ ID NO: 9; (e) HVR-L2 comprising the amino acid sequence of SEQ ID NO: 10; and (f) HVR-L3 comprising the amino acid sequence of SEQ ID NO: 11.

In one aspect, the invention provides an antibody-drug conjugate comprising an antibody that comprises at least one, at least two, or all three VH HVR sequences selected from (a) HVR-H1 comprising the amino acid sequence of SEQ ID NO: 12; (b) HVR-H2 comprising the amino acid sequence of SEQ ID NO: 13; and (c) HVR-H3 comprising the amino acid sequence of SEQ ID NO: 14. In a further embodiment, the antibody comprises (a) HVR-H1 comprising the amino acid sequence of SEQ ID NO: 12; (b) HVR-H2 comprising the amino acid sequence of SEQ ID NO: 13; and (c) HVR-H3 comprising the amino acid sequence of SEQ ID NO: 14.

In another aspect, the invention provides an antibody-drug conjugate comprising an antibody that comprises at least one, at least two, or all three VL HVR sequences selected from (a) HVR-L1 comprising the amino acid sequence of SEQ ID NO: 9; (b) HVR-L2 comprising the amino acid sequence of SEQ ID NO: 10; and (c) HVR-L3 comprising the amino acid sequence of SEQ ID NO: 11. In one embodiment, the antibody comprises (a) HVR-L1 comprising the amino acid sequence of SEQ ID NO: 9; (b) HVR-L2 comprising the amino acid sequence of SEQ ID NO: 10; and (c) HVR-L3 comprising the amino acid sequence of SEQ ID NO: 11.

In another aspect, an antibody-drug conjugate of the invention comprises an antibody comprising (a) a VH domain comprising at least one, at least two, or all three VH HVR sequences selected from (i) HVR-H1 comprising the amino acid sequence of SEQ ID NO: 12, (ii) HVR-H2 comprising the amino acid sequence of SEQ ID NO: 13, and (iii) HVR-H3 comprising an amino acid sequence selected from SEQ ID NO: 14; and (b) a VL domain comprising at least one, at least two, or all three VL HVR sequences selected from (i) HVR-L1 comprising the amino acid sequence of SEQ ID NO: 9, (ii) HVR-L2 comprising the amino acid sequence of SEQ ID NO: 10, and (c) HVR-L3 comprising the amino acid sequence of SEQ ID NO: 11.

In another aspect, the invention provides an antibody-drug conjugate comprising an antibody that comprises (a) HVR-H1 comprising the amino acid sequence of SEQ ID NO: 12; (b) HVR-H2 comprising the amino acid sequence of SEQ ID NO: 13; (c) HVR-H3 comprising the amino acid sequence of SEQ ID NO: 14; (d) HVR-L1 comprising the amino acid sequence of SEQ ID NO: 9; (e) HVR-L2 comprising the amino acid sequence of SEQ ID NO: 10; and (f) HVR-L3 comprising the amino acid sequence of SEQ ID NO: 11.

In any of the above embodiments, an anti-Ly6E antibody of an antibody-drug conjugate is humanized. In one embodiment, an anti-Ly6E antibody comprises HVRs as in any of the above embodiments, and further comprises a human acceptor framework, e.g. a human immunoglobulin framework or a human consensus framework.

In another aspect, an anti-Ly6E antibody of an antibody-drug conjugate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 8. In certain embodiments, a VH sequence having at least 90%, 91%, 92%, 93%, 94%, 95%, 96%, 97%, 98%, or 99% identity to the amino acid sequence of SEQ ID NO: 8 contains substitutions (e.g., conservative substitutions), insertions, or deletions relative to the reference sequence, but an anti-Ly6E antibody comprising that sequence retains the ability to bind to Ly6E. In certain embodiments, a total of 1 to 10 amino acids have been substituted, inserted and/or deleted in SEQ ID NO: 8. In certain embodiments, a total of 1 to 5 amino acids have been substituted, inserted and/or deleted in SEQ ID NO: 8. In certain embodiments, substitutions, insertions, or deletions occur in regions outside the HVRs (i.e., in the FRs). Optionally, the anti-Ly6E antibody comprises the VH sequence of SEQ ID NO: 8, including post-translational modifications of that sequence. In a particular embodiment, the VH comprises one, two or three HVRs selected from: (a) HVR-H1 comprising the amino acid sequence of SEQ ID NO: 12, (b) HVR-H2 comprising the amino acid sequence of SEQ ID NO: 13, and (c) HVR-H3 comprising the amino acid sequence of SEQ ID NO: 14.

In another aspect, an anti-Ly6E antibody of an antibody-drug conjugate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 7. In certain embodiments, a VL sequence having at least 90%, 91%, 92%, 93%, 94%, 95%, 96%, 97%, 98%, or 99% identity to the amino acid sequence of SEQ ID NO:7 contains substitutions (e.g., conservative substitutions), insertions, or deletions relative to the reference sequence, but an anti-Ly6E antibody comprising that sequence retains the ability to bind to Ly6E. In certain embodiments, a total of 1 to 10 amino acids have been substituted, inserted and/or deleted in SEQ ID NO: 7. In certain embodiments, a total of 1 to 5 amino acids have been substituted, inserted and/or deleted in SEQ ID NO: 7. In certain embodiments, the substitutions, insertions, or deletions occur in regions outside the HVRs (i.e., in the FRs). Optionally, the anti-Ly6E antibody comprises the VL sequence of SEQ ID NO: 7, including post-translational modifications of that sequence. In a particular embodiment, the VL comprises one, two or three HVRs selected from (a) HVR-L1 comprising the amino acid sequence of SEQ ID NO: 9; (b) HVR-L2 comprising the amino acid sequence of SEQ ID NO: 10; and (c) HVR-L3 comprising the amino acid sequence of SEQ ID NO: 11.

In another aspect, an antibody-drug conjugate comprising an anti-Ly6E antibody is provided, wherein the antibody comprises a VH as in any of the embodiments provided above, and a VL as in any of the embodiments provided above.

In one embodiment, an antibody-drug conjugate is provided, wherein the antibody comprises the VH and VL sequences in SEQ ID NO: 8 and SEQ ID NO: 7, respectively, including post-translational modifications of those sequences.

In a further aspect, provided herein are antibody-drug conjugate comprising antibodies that bind to the same epitope as an anti-Ly6E antibody provided herein. For example, in certain embodiments, an immunoconjugate is provided comprising an antibody that binds to the same epitope as an anti-Ly6E antibody comprising a VH sequence of SEQ ID NO: 8 and a VL sequence of SEQ ID NO: 7, respectively.

In a further aspect of the invention, an anti-Ly6E antibody of an antibody-drug conjugate according to any of the above embodiments is a monoclonal antibody, including a human antibody. In one embodiment, an anti-Ly6E antibody of an antibody-drug conjugate is an antibody fragment, e.g., a Fv, Fab, Fab′, scFv, diabody, or F(ab′)₂ fragment. In another embodiment, the antibody is a substantially full length antibody, e.g., an IgG1 antibody, IgG2a antibody or other antibody class or isotype as defined herein. In some embodiments, an immunconjugate (ADC) comprises an anti-Ly6E antibody comprising a heavy chain and a light chain comprising the amino acid sequences of SEQ ID NO: 16 and 15, respectively.

Table of Ly6E AntibodySequences

| SEQ ID NO | Description | Sequence |
|---|---|---|
| 7 | anti-Ly6E | DIQMTQSPSS LSASVGDRVT ITCSASQGIS NYLNWYQQKP |
| | antibody | GKTVKLLIYY TSNLHSGVPS RFSGSGSGTD YTLTISSLQP |
| | hu9B12 V12 | EDFATYYCQQ YSELPWTFGQ GTKVEIK |
| | light chain | |
| | variable region | |
| 8 | anti-Ly6E | EVQLVESGPA LVKPTQTLTL TCTVSGFSLT |
| | antibody | GYSVNWIRQPPGKAL EWLGMIWGDG STDYNSALKS |
| | hU9B12 V12 | RLTISKDTSK NQVVLTMTNM DPVDTATYYC ARDYYFNYAS |
| | heavy chain | WFAYWGQGTL VTVSS |
| | variable region | |
| 9 | anti-Ly6E | SASQGISNYLN |
| | antibody | |
| | hU9B12 V12 | |
| | HVR-L₁ | |
| 10 | anti-Ly6E | YTSNLHS |
| | antibody | |
| | hU9B12 V12 | |
| | HVR-L₂ | |
| 11 | anti-Ly6E | QQYSELPWT |
| | antibody | |
| | hugth.2 V12 | |
| | HVR-L₃ | |
| 12 | anti-Ly6E | GFSLTGYSVN |
| | antibody | |
| | hugBi2 V12 | |
| | HVR-H₁ | |
| 13 | anti-Ly6E | MIWGDGSTDY NSALKS |
| | antibody | |
| | hU9B12 V12 | |
| | HVR-H₂ | |
| 14 | anti-Ly6E | DYYVNYASWFAY |
| | antibody | |
| | hugB12 V12 | |
| | HVR-H₃ | |
| 15 | anti-Ly6E | DIQMTQSPSS LSASVGDRVT ITCSASQGIS NYLNWYQQKP |
| | antibody | GKTVKLLIYY TSNLHSGVPS RFSGSGSGTD YTLTISSLQP |
| | hU9B12 V12 | EDFATYYCQQ YSELPWTFGQ GTKVEIK RTVAAPSVFIF |
| | KiztgC kappa | PPSDEQLKSG TASVVCLLNN FYPREAKVQW CVDNALQSGN |
| | light chain | SQESVTEQDS KDSTYSLSST LTLSKADYEK HKVYACEVTH |
| | | QGLSSPVTKS FNRGEC |
| 16 | anti-Ly6E | EVQLVESGPA LVKPTQTLTL TCTVSGFSLT GYSVNWIRQP |
| | antibody | PGKALEWLGM IWGDGSTDYN SALKSRLTIS KDTSKNQVVL |
| | hu9B12 V12 | TMTNMDPVDT ATYYCARDYY FNYASWFAYW GQGTLVTVSS |
| | IgG1 heavy | ASTKGPSVFP LAPSSKSTSG GTAALGCLVK DYFPEPVTVS |
| | chain | WNSGALTSGV HTFPAVLQSS GLYSLSSVVT VPSSSLGTQT |
| | | YICNVNHKPS NTKVDKKVEP KSCDKTHTCP PCPAPELLGG |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTIPPV |
| | | LDSDGSFFLY SKLTVDKSRW QQGNVFSCSV MHEALHNHYT |
| | | QKSLSLSPGK |

Anti-HER2 Antibodies

In certain embodiments, an ADC comprises anti-HER2 antibodies. In one embodiment of the invention, an anti-HER2 antibody of an ADC of the invention comprises a humanized anti-HER2 antibody, e.g., huMAb4D5-1, huMAb4D5-2, huMAb4D5-3, huMAb4D5-4, huMAb4D5-5, huMAb4D5-6, huMAb4D5-7 and huMAb4D5-8, as described in Table 3 of U.S. Pat. No. 5,821,337, which is specifically incorporated by reference herein. Those antibodies contain human framework regions with the complementarity-determining regions of a murine antibody (4D5) that binds to HER2. The humanized antibody huMAb4D5-8 is also referred to as trastuzumab, commercially available under the tradename HERCEPTIN®. In another embodiment of the invention, an anti-HER2 antibody of an ADC of the invention comprises a humanized anti-HER2 antibody, e.g., humanized 2C4, as described in U.S. Pat. No. 7,862,817. An exemplary humanized 2C4 antibody is pertuzumab, commercially available under the tradename PERJETA®.

In another embodiment of the invention, an anti-HER2 antibody of an ADC of the invention comprises a humanized 7C2 anti-HER2 antibody. A humanized 7C2 antibody is an anti-HER2 antibody.

In some embodiments, the invention provides an antibody-drug conjugate comprising an anti-HER2 antibody comprising at least one, two, three, four, five, or six HVRs selected from (a) HVR-H1 comprising the amino acid sequence of SEQ ID NO: 22; (b) HVR-H2 comprising the amino acid sequence of SEQ ID NO: 23, 27, or 28; (c) HVR-H3 comprising the amino acid sequence of SEQ ID NO: 24 or 29; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21. In some embodiments, the invention provides an antibody-drug conjugate comprising an anti-HER2 antibody comprising at least one, two, three, four, five, or six HVRs selected from (a) HVR-H1 comprising the amino acid sequence of SEQ ID NO: 22; (b) HVR-H2 comprising the amino acid sequence of SEQ ID NO: 23; (c) HVR-H3 comprising the amino acid sequence of SEQ ID NO: 24; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21.

In one aspect, the invention provides an antibody-drug conjugate comprising an antibody that comprises at least one, at least two, or all three VH HVR sequences selected from (a) HVR-H1 comprising the amino acid sequence of SEQ ID NO: 22; (b) HVR-H2 comprising the amino acid sequence of SEQ ID NO: 23, 27, or 28; and (c) HVR-H3 comprising the amino acid sequence of SEQ ID NO: 24 or 29. In one aspect, the invention provides an immunoconjugate comprising an antibody that comprises at least one, at least two, or all three VH HVR sequences selected from (a) HVR-H1 comprising the amino acid sequence of SEQ ID NO: 22; (b) HVR-H2 comprising the amino acid sequence of SEQ ID NO: 23; and (c) HVR-H3 comprising the amino acid sequence of SEQ ID NO: 24. In a further embodiment, the antibody comprises (a) HVR-H1 comprising the amino acid sequence of SEQ ID NO: 22; (b) HVR-H2 comprising the amino acid sequence of SEQ ID NO: 23, 27, or 28; and (c) HVR-H3 comprising the amino acid sequence of SEQ ID NO: 24 or 29. In a further embodiment, the antibody comprises (a) HVR-H1 comprising the amino acid sequence of SEQ ID NO: 22; (b) HVR-H2 comprising the amino acid sequence of SEQ ID NO: 23; and (c) HVR-H3 comprising the amino acid sequence of SEQ ID NO: 24.

In another aspect, the invention provides an antibody-drug conjugate comprising an antibody that comprises at least one, at least two, or all three VL HVR sequences selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21. In one embodiment, the antibody comprises (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21.

In another aspect, an antibody-drug conjugate of the invention comprises an antibody comprising (a) a VH domain comprising at least one, at least two, or all three VH HVR sequences selected from (i) HVR-H1 comprising the amino acid sequence of SEQ ID NO: 22, (ii) HVR-H2 comprising the amino acid sequence of SEQ ID NO: 23, 27, or 28, and (iii) HVR-H3 comprising an amino acid sequence selected from SEQ ID NO: 24 or 29; and (b) a VL domain comprising at least one, at least two, or all three VL HVR sequences selected from (i) HVR-L1 comprising the amino acid sequence of SEQ ID NO: 19, (ii) HVR-L2 comprising the amino acid sequence of SEQ ID NO: 20, and (c) HVR-L3 comprising the amino acid sequence of SEQ ID NO: 21. In another aspect, an antibody-drug conjugate of the invention comprises an antibody comprising (a) a VH domain comprising at least one, at least two, or all three VH HVR sequences selected from (i) HVR-H1 comprising the amino acid sequence of SEQ ID NO: 22, (ii) HVR-H2 comprising the amino acid sequence of SEQ ID NO: 23, and (iii) HVR-H3 comprising an amino acid sequence selected from SEQ ID NO: 24; and (b) a VL domain comprising at least one, at least two, or all three VL HVR sequences selected from (i) HVR-L1 comprising the amino acid sequence of SEQ ID NO: 19, (ii) HVR-L2 comprising the amino acid sequence of SEQ ID NO: 20, and (c) HVR-L3 comprising the amino acid sequence of SEQ ID NO: 21.

In another aspect, the invention provides an antibody-drug conjugate comprising an antibody that comprises (a) HVR-H1 comprising the amino acid sequence of SEQ ID NO: 22; (b) HVR-H2 comprising the amino acid sequence of SEQ ID NO: 23, 27, or 28; (c) HVR-H3 comprising the amino acid sequence of SEQ ID NO: 24 or 29; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21. In another aspect, the invention provides an antibody-drug conjugate comprising an antibody that comprises (a) HVR-H1 comprising the amino acid sequence of SEQ ID NO: 22; (b) HVR-H2 comprising the amino acid sequence of SEQ ID NO: 23; (c) HVR-H3 comprising the amino acid sequence of SEQ ID NO: 24; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21.

In any of the above embodiments, an anti-HER2 antibody of an antibody-drug conjugate is humanized. In one embodiment, an anti-HER2 antibody of an antibody-drug conjugate comprises HVRs as in any of the above embodiments, and further comprises a human acceptor framework, e.g. a human immunoglobulin framework or a human consensus framework.

In another aspect, an anti-HER2 antibody of an antibody-drug conjugate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 18. In certain embodiments, a VH sequence having at least 90%, 91%, 92%, 93%, 94%, 95%, 96%, 97%, 98%, or 99% identity to the amino acid sequence of SEQ ID NO: 18 contains substitutions (e.g., conservative substitutions), insertions, or deletions relative to the reference sequence, but an anti-HER2 antibody comprising that sequence retains the ability to bind to HER2. In certain embodiments, a total of 1 to 10 amino acids have been substituted, inserted and/or deleted in SEQ ID NO: 18. In certain embodiments, a total of 1 to 5 amino acids have been substituted, inserted and/or deleted in SEQ ID NO: 18. In certain embodiments, substitutions, insertions, or deletions occur in regions outside the HVRs (i.e., in the FRs). Optionally, the anti-HER2 antibody comprises the VH sequence of SEQ ID NO: 18, including post-translational modifications of that sequence. In a particular embodiment, the VH comprises one, two or three HVRs selected from: (a) HVR-H1 comprising the amino acid sequence of SEQ ID NO: 22, (b) HVR-H2 comprising the amino acid sequence of SEQ ID NO: 23, and (c) HVR-H3 comprising the amino acid sequence of SEQ ID NO: 24.

In another aspect, an anti-HER2 antibody of an antibody-drug conjugate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 17. In certain embodiments, a VL sequence having at least 90%, 91%, 92%, 93%, 94%, 95%, 96%, 97%, 98%, or 99% identity to the amino acid sequence of SEQ ID NO: 17 contains substitutions (e.g., conservative substitutions), insertions, or deletions relative to the reference sequence, but an anti-HER2 antibody comprising that sequence retains the ability to bind to HER2. In certain embodiments, a total of 1 to 10 amino acids have been substituted, inserted and/or deleted in SEQ ID NO: 17. In certain embodiments, a total of 1 to 5 amino acids have been substituted, inserted and/or deleted in SEQ ID NO: 17. In certain embodiments, the substitutions, insertions, or deletions occur in regions outside the HVRs (i.e., in the FRs). Optionally, the anti-HER2 antibody comprises the VL sequence of SEQ ID NO: 17, including post-translational modifications of that sequence. In a particular embodiment, the VL comprises one, two or three HVRs selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21.

In another aspect, an antibody-drug conjugate comprising an anti-HER2 antibody is provided, wherein the antibody comprises a VH as in any of the embodiments provided above, and a VL as in any of the embodiments provided above.

In one embodiment, an antibody-drug conjugate comprising an antibody is provided, wherein the antibody comprises the VH and VL sequences in SEQ ID NO: 18 and SEQ ID NO: 17, respectively, including post-translational modifications of those sequences.

In one embodiment, an antibody-drug conjugate comprising an antibody is provided, wherein the antibody comprises the humanized 7C2.v2.2.LA (hu7C2) K149C kappa light chain sequence of SEQ ID NO: 30

In one embodiment, an antibody-drug conjugate comprising an antibody is provided, wherein the antibody comprises the Hu7C2 A118C IgG1 heavy chain sequence of SEQ ID NO: 31 In a further aspect, provided herein are antibody-drug conjugates comprising antibodies that bind to the same epitope as an anti-HER2 antibody provided herein.

For example, in certain embodiments, an immunoconjugate is provided, comprising an antibody that binds to the same epitope as an anti-HER2 antibody comprising a VH sequence of SEQ ID NO: 18 and a VL sequence of SEQ ID NO: 17, respectively.

In a further aspect of the invention, an anti-HER2 antibody of an antibody-drug conjugate according to any of the above embodiments is a monoclonal antibody, including a human antibody. In one embodiment, an anti-HER2 antibody of an immunoconjugate is an antibody fragment, e.g., a Fv, Fab, Fab′, scFv, diabody, or F(ab′)₂ fragment. In another embodiment, an immunoconjugate comprises an antibody that is a substantially full length antibody, e.g., an IgG1 antibody, IgG2a antibody or other antibody class or isotype as defined herein.

Table of humanized 7C2 anti-HER2 antibody sequences

| SEQ ID NO | Description | Sequence |
|---|---|---|
| 17 | Humanized | DIVMTQSPDS LAVSLGERAT INCRASQSVS GSRFTYMHWY QQKPGQPPKL |
| | 7C2.V2.2.LA | LIKYASILES GVPDRFSGSG SGTDFTLTIS SLQAEDVAVY YCQHSWEIPP |
| | (“hu7C2”) light | WTFGQGTKVE IK |
| | chain variable | |
| | region | |
| 18 | Humanized | EVQLVQSGAE VKKPGASVKV SCKASGYSFT GYWMNWVRQA PGQGLEWIGM |
| | 7C2.V2.2.LA | IHPLDAEIRA NQKFRDRVTI TVDTSTSTAY LELSSLRSED TAVYYCARGT |
| | (“hu7C2”) | YDGGFEYWGQ GTLVTVSS |
| | heavy chain | |
| | variable region | |
| 19 | hu7C2 HVR-L₁ | RASQSVSGSRFTYMH |
| 20 | hu7C2 HVR-L₂ | YASILES |
| 21 | hu7C2 HVR-L₃ | QHSWEIPPWT |
| 22 | hu7C2 HVR-H₁ | GYWMN |
| 23 | hu7C2 HVR-H₂ | MIHPLDAEIRANQKFRD |
| 24 | hu7C2 HVR-H₃ | GTYDGGFEY |
| 25 | Humanized | DIVMTQSPDS LAVSLGERAT INCRASQSVS GSRFTYMHWY QQKPGQPPKL |
| | 7C2.V2.2.LA | LIKYASILES GVPDRFSGSG SGTDFTLTIS SLQAEDVAVY YCQHSWEIPP |
| | (hu7C2) kappa | WTFGQGTKVE IKRTVAAPSV FIFPPSDEQL KSGTASVVCL LNNFYPREAK |
| | light chain | VQWKVDNALQ SGNSQESVTE QDSKDSTYSL SSTLTLSKAD YEKHKVYACE |
| | | VTHQGLSSPV TKSFNRGEC |
| 26 | Humanized | EVQLVQSGAE VKKPGASVKV SCKASGYSFT GYWMNWVRQA PGQGLEWIGM |
| | 7C2.V2.2.LA | IHPLDAEIRA NQKFRDRVTI TVDTSTSTAY LELSSLRSED TAVYYCARGT |
| | (hu7C2) IgG₁ | YDGGFEYWGQ GTLVTVSSAS TKGPSVFPLA PSSKSTSGGT AALGCLVKDY |
| | heavy chain | FPEPVTVSWN SGALTSGVHT FPAVLQSSGL YSLSSVVIVP SSSLGTQTYI |
| | | CNVNHKPSNT KVDKKVEPKS CDKTHTCPPC PAPELLGGPS VFLFPPKPKD |
| | | TLMISRTPEV TCVVVDVSHE DPEVKFNWYV DGVEVHNAKT KPREEQYNST |
| | | YRVVSVLTVL HQDWLNGKEY KCKVSNKALP APIEKTISKA KGQPREPQVY |
| | | TLPPSREEMT KNQVSLTCLV KGFYPSDIAV EWESNGQPEN NYKTTPPVLD |
| | | SDGSFFLYSK LTVDKSRWQQ GNVFSCSVMH EALHNHYTQK SLSLSPGK |
| 27 | Hu7C2. | MIHPMDSEIRANQKFRD |
| | V2.1.S₅₃M | |
| | HVR-H₂ | |
| 28 | Hu7C2. | MIHPLDSEIRANQKFRD |
| | V2.1.S₅₃L | |
| | HVR-H₂ | |
| 29 | Hu7C2. | GTYDGGFKY |
| | V2.1.E₁₀₁K | |
| | HVR-H₃ | |
| 30 | Humanized | DIVMTQSPDS LAVSLGERAT INCRASQSVS GSRFTYMHWY QQKPGQPPKL |
| | 7C2.V2.2.LA | LIKYASILES GVPDRFSGSG SGTDFTLTIS SLQAEDVAVY YCQHSWEIPP |
| | (hu7C2) K₁₄₉C | WTFGQGTKVE IKRTVAAPSV FIFPPSDEQL KSGTASVVCL LNNFYPREAK |
| | kappa light | VQWCVDNALQ SGNSQESVTE QDSKDSTYSL SSTLTLSKAD YEKHKVYACE |
| | chain | VTHQGLSSPV TKSFNRGEC |
| 31 | Humanized | EVQLVQSGAE VKKPGASVKV SCKASGYSFT GYWMNWVRQA PGQGLEWIGM |
| | 7C2.V2.2.LA | IHPLDAEIRA NQKFRDRVTI TVDTSTSTAY LELSSLRSED TAVYYCARGT |
| | (hu7C2) A₁₁8C | YDGGFEYWGQ GTLVTVSSCS TKGPSVFPLA PSSKSTSGGT AALGCLVKDY |
| | IgG₁ heavy | FPEPVIVSWN SGALTSGVHT FPAVLQSSGL YSLSSVVTVP SSSLGTQTYI |
| | chain | CNVNHKPSNT KVDKKVEPKS CDKTHTCPPC PAPELLGGPS VFLFPPKPKD |
| | | TLMISRTPEV TCVVVDVSHE DPEVKFNWYV DGVEVHNAKT KPREEQYNST |
| | | YRVVSVLTVL HQDWLNGKEY KCKVSNKALP APIEKTISKA KGQPREPQVY |
| | | TLPPSREEMT KNQVSLTCLV KGFYPSDIAV EWESNGQPEN NYKTTPPVLD |
| | | SDGSFFLYSK LTVDKSRWQQ GNVFSCSVMH EALHNHYTQK SLSLSPGK |

Anti-MUC16 Antibodies

In certain embodiments, an ADC comprises anti-MUC16 antibodies.

In some embodiments, the invention provides an antibody-drug conjugate comprising an anti-MUC16 antibody comprising at least one, two, three, four, five, or six HVRs selected from (a) HVR-H1 comprising the amino acid sequence of SEQ ID NO: 35; (b) HVR-H2 comprising the amino acid sequence of SEQ ID NO: 36; (c) HVR-H3 comprising the amino acid sequence of SEQ ID NO: 37; (d) HVR-L1 comprising the amino acid sequence of SEQ ID NO: 32; (e) HVR-L2 comprising the amino acid sequence of SEQ ID NO: 33 and (f) HVR-L3 comprising the amino acid sequence of SEQ ID NO: 34.

In one aspect, the invention provides an antibody-drug conjugate comprising an antibody that comprises at least one, at least two, or all three VH HVR sequences selected from (a) HVR-H1 comprising the amino acid sequence of SEQ ID NO: 35; (b) HVR-H2 comprising the amino acid sequence of SEQ ID NO: 36; (c) HVR-H3 comprising the amino acid sequence of SEQ ID NO: 37. In a further embodiment, the antibody comprises (a) HVR-H1 comprising the amino acid sequence of SEQ ID NO: 35; (b) HVR-H2 comprising the amino acid sequence of SEQ ID NO: 36; (c) HVR-H3 comprising the amino acid sequence of SEQ ID NO: 37.

In another aspect, the invention provides an antibody-drug conjugate comprising an antibody that comprises at least one, at least two, or all three VL HVR sequences selected from (a) HVR-L1 comprising the amino acid sequence of SEQ ID NO: 32; (b) HVR-L2 comprising the amino acid sequence of SEQ ID NO: 33; and (c) HVR-L3 comprising the amino acid sequence of SEQ ID NO: 34. In one embodiment, the antibody comprises (a) HVR-L1 comprising the amino acid sequence of SEQ ID NO: 32; (b) HVR-L2 comprising the amino acid sequence of SEQ ID NO: 33; and (c) HVR-L3 comprising the amino acid sequence of SEQ ID NO: 34.

In another aspect, an antibody-drug conjugate of the invention comprises an antibody comprising (a) a VH domain comprising at least one, at least two, or all three VH HVR sequences selected from (i) HVR-H1 comprising the amino acid sequence of SEQ ID NO: 35, (ii) HVR-H2 comprising the amino acid sequence of SEQ ID NO: 36, and (iii) HVR-H3 comprising an amino acid sequence selected from SEQ ID NO: 37; and (b) a VL domain comprising at least one, at least two, or all three VL HVR sequences selected from (i) HVR-L1 comprising the amino acid sequence of SEQ ID NO: 32, (ii) HVR-L2 comprising the amino acid sequence of SEQ ID NO: 33, and (c) HVR-L3 comprising the amino acid sequence of SEQ ID NO: 34.

In another aspect, the invention provides an antibody-drug conjugate comprising an antibody that comprises (a) HVR-H1 comprising the amino acid sequence of SEQ ID NO: 35 (b) HVR-H2 comprising the amino acid sequence of SEQ ID NO: 36; (c) HVR-H3 comprising the amino acid sequence of SEQ ID NO: 37; (d) HVR-L1 comprising the amino acid sequence of SEQ ID NO: 32; (e) HVR-L2 comprising the amino acid sequence of SEQ ID NO: 33; and (f) HVR-L3 comprising the amino acid sequence of SEQ ID NO: 34.

In any of the above embodiments, an anti-MUC16 antibody of an antibody-drug conjugate is humanized. In one embodiment, an anti-MUC16 antibody comprises HVRs as in any of the above embodiments, and further comprises a human acceptor framework, e.g. a human immunoglobulin framework or a human consensus framework.

In another aspect, an anti-MUC16 antibody of an antibody-drug conjugate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 39. In certain embodiments, a VH sequence having at least 90%, 91%, 92%, 93%, 94%, 95%, 96%, 97%, 98%, or 99% identity to the amino acid sequence of SEQ ID NO: 39 contains substitutions (e.g., conservative substitutions), insertions, or deletions relative to the reference sequence, but an anti-MUC16 antibody comprising that sequence retains the ability to bind to MUC16. In certain embodiments, a total of 1 to 10 amino acids have been substituted, inserted and/or deleted in SEQ ID NO: 39. In certain embodiments, a total of 1 to 5 amino acids have been substituted, inserted and/or deleted in SEQ ID NO: 39. In certain embodiments, substitutions, insertions, or deletions occur in regions outside the HVRs (i.e., in the FRs). Optionally, the anti-MUC16 antibody comprises the VH sequence of SEQ ID NO: 39, including post-translational modifications of that sequence. In a particular embodiment, the VH comprises one, two or three HVRs selected from: (a) HVR-H1 comprising the amino acid sequence of SEQ ID NO: 35, (b) HVR-H2 comprising the amino acid sequence of SEQ ID NO: 36, and (c) HVR-H3 comprising the amino acid sequence of SEQ ID NO: 37.

In another aspect, an anti-MUC16 antibody of an antibody-drug conjugate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 38. In certain embodiments, a VL sequence having at least 90%, 91%, 92%, 93%, 94%, 95%, 96%, 97%, 98%, or 99% identity to the amino acid sequence of SEQ ID NO:38 contains substitutions (e.g., conservative substitutions), insertions, or deletions relative to the reference sequence, but an anti-MUC16 antibody comprising that sequence retains the ability to bind to MUC16. In certain embodiments, a total of 1 to 10 amino acids have been substituted, inserted and/or deleted in SEQ ID NO: 38. In certain embodiments, a total of 1 to 5 amino acids have been substituted, inserted and/or deleted in SEQ ID NO: 38. In certain embodiments, the substitutions, insertions, or deletions occur in regions outside the HVRs (i.e., in the FRs). Optionally, the anti-MUC16 antibody comprises the VL sequence of SEQ ID NO: 38, including post-translational modifications of that sequence. In a particular embodiment, the VL comprises one, two or three HVRs selected from (a) HVR-L1 comprising the amino acid sequence of SEQ ID NO: 32; (b) HVR-L2 comprising the amino acid sequence of SEQ ID NO: 33; and (c) HVR-L3 comprising the amino acid sequence of SEQ ID NO: 34.

In another aspect, an antibody-drug conjugate comprising an anti-MUC16 antibody is provided, wherein the antibody comprises a VH as in any of the embodiments provided above, and a VL as in any of the embodiments provided above.

In one embodiment, an antibody-drug conjugate is provided, wherein the antibody comprises the VH and VL sequences in SEQ ID NO: 39 and SEQ ID NO: 38, respectively, including post-translational modifications of those sequences.

In a further aspect, provided herein are antibody-drug conjugate comprising antibodies that bind to the same epitope as an anti-MUC16 antibody provided herein. For example, in certain embodiments, an immunoconjugate is provided comprising an antibody that binds to the same epitope as an anti-MUC16 antibody comprising a VH sequence of SEQ ID NO: 39 and a VL sequence of SEQ ID NO: 38, respectively.

In a further aspect of the invention, an anti-MUC16 antibody of an antibody-drug conjugate according to any of the above embodiments is a monoclonal antibody, including a human antibody. In one embodiment, an anti-MUC16 antibody of an antibody-drug conjugate is an antibody fragment, e.g., a Fv, Fab, Fab′, scFv, diabody, or F(ab′)₂ fragment. In another embodiment, the antibody is a substantially full length antibody, e.g., an IgG1 antibody, IgG2a antibody or other antibody class or isotype as defined herein.

Table of MUC16 Antibody Sequences

| SEQ ID NO | Description | Sequence |
|---|---|---|
| 32 | Anti-Muc16 | KASDLIHNWL A |
| | antibody | |
| | HVR-L₁ | |
| 33 | Anti-Muc16 | YGATSLET |
| | antibody | |
| | HVR-L₂ | |
| 34 | Anti-Muc16 | QQYWITPFT |
| | antibody | |
| | HVR-L₃ | |
| 35 | Anti-Muc16 | GYSITNDYAW N |
| | antibody | |
| | HVR-H₁ | |
| 36 | Anti-Muc16 | GYISYSGYIT YNPSLKS |
| | antibody | |
| | HVR-H₂ | |
| 37 | Anti-Muc16 | ARWASGLDY |
| | antibody | |
| | HVR-H₃ | |
| 38 | Anti-Muc16 | DIQMTQSPSS LSASVGDRVT ITCKASDLIH NWLAWYQQKP |
| | antibody light | GKAPKLLIYG ATSLETGVPS RFSGSGSGTD FTLTISSLQP |
| | chain variable | EDFATYYCQQ YWITPFTFGQ GTKVEIKR |
| | region | |
| 39 | Anti-Muc16 | EVQLVESGGG LVQPGGSLRL SCAASGYSIT |
| | antibody heavy | NDYAWNWVRQ APGKGLEWVG YISYSGYTTY |
| | chain variable | NPSLKSRFTI SRDTSKNTLY LQMNSLRAED |
| | region | TAVYYCARWA SGLDYWGQGT LVTVSS |

Anti-STEAP-1 Antibodies

In certain embodiments, an ADC comprises anti-STEAP-1 antibodies.

In some embodiments, the invention provides an antibody-drug conjugate comprising an anti-STEAP-1 antibody comprising at least one, two, three, four, five, or six HVRs selected from (a) HVR-H1 comprising the amino acid sequence of SEQ ID NO: 40; (b) HVR-H2 comprising the amino acid sequence of SEQ ID NO: 41; (c) HVR-H3 comprising the amino acid sequence of SEQ ID NO: 42; (d) HVR-L1 comprising the amino acid sequence of SEQ ID NO: 43; (e) HVR-L2 comprising the amino acid sequence of SEQ ID NO: 44 and (f) HVR-L3 comprising the amino acid sequence of SEQ ID NO: 45.

In one aspect, the invention provides an antibody-drug conjugate comprising an antibody that comprises at least one, at least two, or all three VH HVR sequences selected from (a) HVR-H1 comprising the amino acid sequence of SEQ ID NO: 40; (b) HVR-H2 comprising the amino acid sequence of SEQ ID NO: 41; (c) HVR-H3 comprising the amino acid sequence of SEQ ID NO: 42. In a further embodiment, the antibody comprises (a) HVR-H1 comprising the amino acid sequence of SEQ ID NO: 40; (b) HVR-H2 comprising the amino acid sequence of SEQ ID NO: 41; (c) HVR-H3 comprising the amino acid sequence of SEQ ID NO: 42.

In another aspect, the invention provides an antibody-drug conjugate comprising an antibody that comprises at least one, at least two, or all three VL HVR sequences selected from (a) HVR-L1 comprising the amino acid sequence of SEQ ID NO: 43; (b) HVR-L2 comprising the amino acid sequence of SEQ ID NO: 44; and (c) HVR-L3 comprising the amino acid sequence of SEQ ID NO: 45. In one embodiment, the antibody comprises (a) HVR-L1 comprising the amino acid sequence of SEQ ID NO: 43; (b) HVR-L2 comprising the amino acid sequence of SEQ ID NO: 44; and (c) HVR-L3 comprising the amino acid sequence of SEQ ID NO: 45.

In another aspect, an antibody-drug conjugate of the invention comprises an antibody comprising (a) a VH domain comprising at least one, at least two, or all three VH HVR sequences selected from (i) HVR-H1 comprising the amino acid sequence of SEQ ID NO: 40, (ii) HVR-H2 comprising the amino acid sequence of SEQ ID NO: 41, and (iii) HVR-H3 comprising an amino acid sequence selected from SEQ ID NO: 42; and (b) a VL domain comprising at least one, at least two, or all three VL HVR sequences selected from (i) HVR-L1 comprising the amino acid sequence of SEQ ID NO: 43, (ii) HVR-L2 comprising the amino acid sequence of SEQ ID NO: 44, and (c) HVR-L3 comprising the amino acid sequence of SEQ ID NO: 45.

In another aspect, the invention provides an antibody-drug conjugate comprising an antibody that comprises (a) HVR-H1 comprising the amino acid sequence of SEQ ID NO: 40 (b) HVR-H2 comprising the amino acid sequence of SEQ ID NO: 41; (c) HVR-H3 comprising the amino acid sequence of SEQ ID NO: 42; (d) HVR-L1 comprising the amino acid sequence of SEQ ID NO: 43; (e) HVR-L2 comprising the amino acid sequence of SEQ ID NO: 44; and (f) HVR-L3 comprising the amino acid sequence of SEQ ID NO: 45.

In any of the above embodiments, an anti-STEAP-1 antibody of an antibody-drug conjugate is humanized. In one embodiment, an anti-STEAP-1 antibody comprises HVRs as in any of the above embodiments, and further comprises a human acceptor framework, e.g. a human immunoglobulin framework or a human consensus framework.

In another aspect, an anti-STEAP-1 antibody of an antibody-drug conjugate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 46. In certain embodiments, a VH sequence having at least 90%, 91%, 92%, 93%, 94%, 95%, 96%, 97%, 98%, or 99% identity to the amino acid sequence of SEQ ID NO: 46 contains substitutions (e.g., conservative substitutions), insertions, or deletions relative to the reference sequence, but an anti-STEAP-1 antibody comprising that sequence retains the ability to bind to STEAP-1. In certain embodiments, a total of 1 to 10 amino acids have been substituted, inserted and/or deleted in SEQ ID NO: 46. In certain embodiments, a total of 1 to 5 amino acids have been substituted, inserted and/or deleted in SEQ ID NO: 46. In certain embodiments, substitutions, insertions, or deletions occur in regions outside the HVRs (i.e., in the FRs). Optionally, the anti-STEAP-1 antibody comprises the VH sequence of SEQ ID NO: 46, including post-translational modifications of that sequence. In a particular embodiment, the VH comprises one, two or three HVRs selected from: (a) HVR-H1 comprising the amino acid sequence of SEQ ID NO: 40, (b) HVR-H2 comprising the amino acid sequence of SEQ ID NO: 41, and (c) HVR-H3 comprising the amino acid sequence of SEQ ID NO: 42.

In another aspect, an anti-STEAP-1 antibody of an antibody-drug conjugate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 47. In certain embodiments, a VL sequence having at least 90%, 91%, 92%, 93%, 94%, 95%, 96%, 97%, 98%, or 99% identity to the amino acid sequence of SEQ ID NO: 47 contains substitutions (e.g., conservative substitutions), insertions, or deletions relative to the reference sequence, but an anti-STEAP-1 antibody comprising that sequence retains the ability to bind to STEAP-1. In certain embodiments, a total of 1 to 10 amino acids have been substituted, inserted and/or deleted in SEQ ID NO: 47 In certain embodiments, a total of 1 to 5 amino acids have been substituted, inserted and/or deleted in SEQ ID NO: 47. In certain embodiments, the substitutions, insertions, or deletions occur in regions outside the HVRs (i.e., in the FRs). Optionally, the anti-STEAP-1 antibody comprises the VL sequence of SEQ ID NO: 47, including post-translational modifications of that sequence. In a particular embodiment, the VL comprises one, two or three HVRs selected from (a) HVR-L1 comprising the amino acid sequence of SEQ ID NO: 43; (b) HVR-L2 comprising the amino acid sequence of SEQ ID NO: 44; and (c) HVR-L3 comprising the amino acid sequence of SEQ ID NO: 45.

In another aspect, an antibody-drug conjugate comprising an anti-STEAP-1 antibody is provided, wherein the antibody comprises a VH as in any of the embodiments provided above, and a VL as in any of the embodiments provided above.

In one embodiment, an antibody-drug conjugate is provided, wherein the antibody comprises the VH and VL sequences in SEQ ID NO: 46 and SEQ ID NO: 47, respectively, including post-translational modifications of those sequences.

In a further aspect, provided herein are antibody-drug conjugate comprising antibodies that bind to the same epitope as an anti-STEAP-1 antibody provided herein. For example, in certain embodiments, an immunoconjugate is provided comprising an antibody that binds to the same epitope as an anti-STEAP-1 antibody comprising a VH sequence of SEQ ID NO: 46 and a VL sequence of SEQ ID NO: 47, respectively.

In a further aspect of the invention, an anti-STEAP-1 antibody of an antibody-drug conjugate according to any of the above embodiments is a monoclonal antibody, including a human antibody. In one embodiment, an anti-STEAP-1 antibody of an antibody-drug conjugate is an antibody fragment, e.g., a Fv, Fab, Fab′, scFv, diabody, or F(ab′)₂ fragment. In another embodiment, the antibody is a substantially full length antibody, e.g., an IgG1 antibody, IgG2a antibody or other antibody class or isotype as defined herein.

Table of STEAP Antibody Sequences

| SEQ ID NO | Description | Sequence |
|---|---|---|
| 40 | Anti-STEAP-₁ | GYSITSDYAW N |
| | HVR-H₁ | |
| 41 | Anti-STEAP-₁ | GYISNSGSTS YNPSLKS |
| | HVR-H₂ | |
| 42 | Anti-STEAP-₁ | ERNYDYDDYY YAMDY |
| | HVR-H₃ | |
| 43 | Anti-STEAP-1 | KSSQSLLYRS NQKNYLA |
| | HVR-L₁ | |
| 44 | Anti-STEAP-₁ | WASTRES |
| | HVR-L₂ | |
| 45 | Anti-STEAP-₁ | QQYYNYPRT |
| | HVR-L₃ | |
| 46 | Anti-STEAP-₁ | EVQLVESGGG LVQPGGSLRL SCAVSGYSIT SDYAWNWVRQ |
| | heavy chain | APGKGLEWVG YISNSGSTSY NPSLKSRFTI SRDTSKNTLY |
| | variable region | LQMNSLRAED TAVYYCARER NYDYDDYYYA MDYWGQGTLV |
| | | TVSS |
| 47 | Anti-STEAP-₁ | DIQMTQSPSS LSASVGDRVT ITCKSSQSLL YRSNQKNYLA |
| | light chain | WYQQKPGKAP KLLIYWASTR ESGVPSRFSG SGSGTDFTLT |
| | variable region | ISSLQPEDFA TYYCQQYYNY PRTFGQGTKV EIK |

Anti-NaPi2b Antibodies

In certain embodiments, an ADC comprises anti-NaPi2b antibodies. In some embodiments, the invention provides an antibody-drug conjugate comprising an anti-NaPi2b antibody comprising at least one, two, three, four, five, or six HVRs selected from (a) HVR-H1 comprising the amino acid sequence of SEQ ID NO: 48; (b) HVR-H2 comprising the amino acid sequence of SEQ ID NO: 49; (c) HVR-H3 comprising the amino acid sequence of SEQ ID NO: 50; (d) HVR-L1 comprising the amino acid sequence of SEQ ID NO: 51; (e) HVR-L2 comprising the amino acid sequence of SEQ ID NO: 52 and (f) HVR-L3 comprising the amino acid sequence of SEQ ID NO: 53.

In one aspect, the invention provides an antibody-drug conjugate comprising an antibody that comprises at least one, at least two, or all three VH HVR sequences selected from (a) HVR-H1 comprising the amino acid sequence of SEQ ID NO: 48; (b) HVR-H2 comprising the amino acid sequence of SEQ ID NO: 49; (c) HVR-H3 comprising the amino acid sequence of SEQ ID NO: 50. In a further embodiment, the antibody comprises (a) HVR-H1 comprising the amino acid sequence of SEQ ID NO: 48; (b) HVR-H2 comprising the amino acid sequence of SEQ ID NO: 49; (c) HVR-H3 comprising the amino acid sequence of SEQ ID NO: 50.

In another aspect, the invention provides an antibody-drug conjugate comprising an antibody that comprises at least one, at least two, or all three VL HVR sequences selected from (a) HVR-L1 comprising the amino acid sequence of SEQ ID NO: 51; (b) HVR-L2 comprising the amino acid sequence of SEQ ID NO: 52; and (c) HVR-L3 comprising the amino acid sequence of SEQ ID NO: 53. In one embodiment, the antibody comprises (a) HVR-L1 comprising the amino acid sequence of SEQ ID NO: 51; (b) HVR-L2 comprising the amino acid sequence of SEQ ID NO: 52; and (c) HVR-L3 comprising the amino acid sequence of SEQ ID NO: 53.

In another aspect, an antibody-drug conjugate of the invention comprises an antibody comprising (a) a VH domain comprising at least one, at least two, or all three VH HVR sequences selected from (i) HVR-H1 comprising the amino acid sequence of SEQ ID NO: 48, (ii) HVR-H2 comprising the amino acid sequence of SEQ ID NO: 49, and (iii) HVR-H3 comprising an amino acid sequence selected from SEQ ID NO: 50; and (b) a VL domain comprising at least one, at least two, or all three VL HVR sequences selected from (i) HVR-L1 comprising the amino acid sequence of SEQ ID NO: 51, (ii) HVR-L2 comprising the amino acid sequence of SEQ ID NO: 52, and (c) HVR-L3 comprising the amino acid sequence of SEQ ID NO: 53.

In another aspect, the invention provides an antibody-drug conjugate comprising an antibody that comprises (a) HVR-H1 comprising the amino acid sequence of SEQ ID NO: 48 (b) HVR-H2 comprising the amino acid sequence of SEQ ID NO: 49; (c) HVR-H3 comprising the amino acid sequence of SEQ ID NO: 50; (d) HVR-L1 comprising the amino acid sequence of SEQ ID NO: 51; (e) HVR-L2 comprising the amino acid sequence of SEQ ID NO: 52; and (f) HVR-L3 comprising the amino acid sequence of SEQ ID NO: 53.

In any of the above embodiments, an anti-NaPi2b antibody of an antibody-drug conjugate is humanized. In one embodiment, an anti-NaPi2b antibody comprises HVRs as in any of the above embodiments, and further comprises a human acceptor framework, e.g. a human immunoglobulin framework or a human consensus framework.

In another aspect, an anti-NaPi2b antibody of an antibody-drug conjugate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 54. In certain embodiments, a VH sequence having at least 90%, 91%, 92%, 93%, 94%, 95%, 96%, 97%, 98%, or 99% identity to the amino acid sequence of SEQ ID NO: 54 contains substitutions (e.g., conservative substitutions), insertions, or deletions relative to the reference sequence, but an anti-NaPi2b antibody comprising that sequence retains the ability to bind to NaPi2b. In certain embodiments, a total of 1 to 10 amino acids have been substituted, inserted and/or deleted in SEQ ID NO: 54. In certain embodiments, a total of 1 to 5 amino acids have been substituted, inserted and/or deleted in SEQ ID NO: 54. In certain embodiments, substitutions, insertions, or deletions occur in regions outside the HVRs (i.e., in the FRs). Optionally, the anti-NaPi2b antibody comprises the VH sequence of SEQ ID NO: 54, including post-translational modifications of that sequence. In a particular embodiment, the VH comprises one, two or three HVRs selected from: (a) HVR-H1 comprising the amino acid sequence of SEQ ID NO: 48, (b) HVR-H2 comprising the amino acid sequence of SEQ ID NO: 49, and (c) HVR-H3 comprising the amino acid sequence of SEQ ID NO: 50.

In another aspect, an anti-NaPi2b antibody of an antibody-drug conjugate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 55. In certain embodiments, a VL sequence having at least 90%, 91%, 92%, 93%, 94%, 95%, 96%, 97%, 98%, or 99% identity to the amino acid sequence of SEQ ID NO: 55 contains substitutions (e.g., conservative substitutions), insertions, or deletions relative to the reference sequence, but an anti-NaPi2b antibody comprising that sequence retains the ability to bind to anti-NaPi2b. In certain embodiments, a total of 1 to 10 amino acids have been substituted, inserted and/or deleted in SEQ ID NO: 55. In certain embodiments, a total of 1 to 5 amino acids have been substituted, inserted and/or deleted in SEQ ID NO: 55. In certain embodiments, the substitutions, insertions, or deletions occur in regions outside the HVRs (i.e., in the FRs). Optionally, the anti-NaPi2b antibody comprises the VL sequence of SEQ ID NO: 55, including post-translational modifications of that sequence. In a particular embodiment, the VL comprises one, two or three HVRs selected from (a) HVR-L1 comprising the amino acid sequence of SEQ ID NO: 51; (b) HVR-L2 comprising the amino acid sequence of SEQ ID NO: 52; and (c) HVR-L3 comprising the amino acid sequence of SEQ ID NO: 53.

In another aspect, an antibody-drug conjugate comprising an anti-NaPi2b antibody is provided, wherein the antibody comprises a VH as in any of the embodiments provided above, and a VL as in any of the embodiments provided above.

In one embodiment, an antibody-drug conjugate is provided, wherein the antibody comprises the VH and VL sequences in SEQ ID NO: 54 and SEQ ID NO: 55, respectively, including post-translational modifications of those sequences.

In a further aspect, provided herein are antibody-drug conjugate comprising antibodies that bind to the same epitope as an anti-NaPi2b antibody provided herein. For example, in certain embodiments, an immunoconjugate is provided comprising an antibody that binds to the same epitope as an anti-NaPi2b antibody comprising a VH sequence of SEQ ID NO: 54 and a VL sequence of SEQ ID NO: 55, respectively.

In a further aspect of the invention, an anti-NaPi2b antibody of an antibody-drug conjugate according to any of the above embodiments is a monoclonal antibody, including a human antibody. In one embodiment, an anti-NaPi2b antibody of an antibody-drug conjugate is an antibody fragment, e.g., a Fv, Fab, Fab′, scFv, diabody, or F(ab′)₂ fragment. In another embodiment, the antibody is a substantially full length antibody, e.g., an IgG1 antibody, IgG2a antibody or other antibody class or isotype as defined herein.

Table of NaPi2b Antibody Sequences

| SEQ ID NO | Description | Sequence |
|---|---|---|
| 48 | Anti-NaPi2b | GFSFSDFAMS |
| | HVR-H₁ | |
| 49 | Anti-NaPi2b | ATIGR VAFHTYYPDSMKG |
| | HVR-H₂ | |
| 50 | Anti-NaPi2b | ARHRGFDVGHFDF |
| | HVR-H₃ | |
| 51 | Anti-NaPi2b | RSSETL VHSSGNTYLE |
| | HVR-L₁ | |
| 52 | Anti-NaPi2b | RVSNRFS |
| | HVR-L₂ | |
| 53 | Anti-NaPi2b | FQGSFNPLT |
| | HVR-L₃ | |
| 54 | Anti-NaPi2b | EVQLVESGGGL VQPGGSLRLSCAASGFSFSDFAMSWV |
| | heavy chain | RQAPGKGLEWVATIGRVAFHTYYPDSMKGRFTISRDNSKN |
| | variable region | TLYLQMNSLRAEDTAVYYCARHRGFDVGHFDFWGQGTLVT |
| | | VSS |
| 55 | Anti-NaPi2b | DIQMTQSPSSLSASVGDRVTITCRSSETL VHSSGNTYLE |
| | light chain | WYQQKPGKAPKLLIYRVSNRFSGVPSRFSGSGSGTDFTLT |
| | variable region | ISSLQPEDFATYYCFQGSFNPLTFGQGTKVEIKR |

Anti-CD79b Antibodies

In certain embodiments, an ADC comprises anti-CD79b antibodies. In some embodiments, the invention provides an antibody-drug conjugate comprising an anti-CD79b antibody comprising at least one, two, three, four, five, or six HVRs selected from (a) HVR-H1 comprising the amino acid sequence of SEQ ID NO: 58; (b) HVR-H2 comprising the amino acid sequence of SEQ ID NO: 59; (c) HVR-H3 comprising the amino acid sequence of SEQ ID NO: 60; (d) HVR-L1 comprising the amino acid sequence of SEQ ID NO: 61; (e) HVR-L2 comprising the amino acid sequence of SEQ ID NO: 62; and (f) HVR-L3 comprising the amino acid sequence of SEQ ID NO: 63.

In one aspect, the invention provides an antibody-drug conjugate comprising an antibody that comprises at least one, at least two, or all three VH HVR sequences selected from (a) HVR-H1 comprising the amino acid sequence of SEQ ID NO: 58; (b) HVR-H2 comprising the amino acid sequence of SEQ ID NO: 59; and (c) HVR-H3 comprising the amino acid sequence of SEQ ID NO: 60. In a further embodiment, the antibody comprises (a) HVR-H1 comprising the amino acid sequence of SEQ ID NO: 58; (b) HVR-H2 comprising the amino acid sequence of SEQ ID NO: 59; and (c) HVR-H3 comprising the amino acid sequence of SEQ ID NO: 60.

In another aspect, the invention provides an antibody-drug conjugate comprising an antibody that comprises at least one, at least two, or all three VL HVR sequences selected from (a) HVR-L1 comprising the amino acid sequence of SEQ ID NO: 61; (b) HVR-L2 comprising the amino acid sequence of SEQ ID NO: 62; and (c) HVR-L3 comprising the amino acid sequence of SEQ ID NO: 63. In one embodiment, the antibody comprises (a) HVR-L1 comprising the amino acid sequence of SEQ ID NO: 61; (b) HVR-L2 comprising the amino acid sequence of SEQ ID NO: 62; and (c) HVR-L3 comprising the amino acid sequence of SEQ ID NO: 63.

In another aspect, an antibody-drug conjugate of the invention comprises an antibody comprising (a) a VH domain comprising at least one, at least two, or all three VH HVR sequences selected from (i) HVR-H1 comprising the amino acid sequence of SEQ ID NO: 58, (ii) HVR-H2 comprising the amino acid sequence of SEQ ID NO: 59, and (iii) HVR-H3 comprising an amino acid sequence selected from SEQ ID NO: 60; and (b) a VL domain comprising at least one, at least two, or all three VL HVR sequences selected from (i) HVR-L1 comprising the amino acid sequence of SEQ ID NO: 61, (ii) HVR-L2 comprising the amino acid sequence of SEQ ID NO: 62, and (iii) HVR-L3 comprising the amino acid sequence of SEQ ID NO: 63.

In another aspect, the invention provides an antibody-drug conjugate comprising an antibody that comprises (a) HVR-H1 comprising the amino acid sequence of SEQ ID NO: 58; (b) HVR-H2 comprising the amino acid sequence of SEQ ID NO: 59; (c) HVR-H3 comprising the amino acid sequence of SEQ ID NO: 60; (d) HVR-L1 comprising the amino acid sequence of SEQ ID NO: 61; (e) HVR-L2 comprising the amino acid sequence of SEQ ID NO: 62; and (f) HVR-L3 comprising the amino acid sequence of SEQ ID NO: 63.

In any of the above embodiments, an anti-CD79b antibody of an antibody-drug conjugate is humanized. In one embodiment, an anti-CD79b antibody comprises HVRs as in any of the above embodiments, and further comprises a human acceptor framework, e.g. a human immunoglobulin framework or a human consensus framework.

In another aspect, an anti-CD79b antibody of an antibody-drug conjugate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 56. In certain embodiments, a VH sequence having at least 90%, 91%, 92%, 93%, 94%, 95%, 96%, 97%, 98%, or 99% identity to the amino acid sequence of SEQ ID NO: 56 contains substitutions (e.g., conservative substitutions), insertions, or deletions relative to the reference sequence, but an anti-CD79b antibody comprising that sequence retains the ability to bind to CD79b. In certain embodiments, a total of 1 to 10 amino acids have been substituted, inserted and/or deleted in SEQ ID NO: 56. In certain embodiments, a total of 1 to 5 amino acids have been substituted, inserted and/or deleted in SEQ ID NO: 56. In certain embodiments, substitutions, insertions, or deletions occur in regions outside the HVRs (i.e., in the FRs). Optionally, the anti-CD79b antibody comprises the VH sequence of SEQ ID NO: 8, including post-translational modifications of that sequence. In a particular embodiment, the VH comprises one, two or three HVRs selected from: (a) HVR-H1 comprising the amino acid sequence of SEQ ID NO: 58, (b) HVR-H2 comprising the amino acid sequence of SEQ ID NO: 59, and (c) HVR-H3 comprising the amino acid sequence of SEQ ID NO: 60.

In another aspect, an anti-CD79b antibody of an antibody-drug conjugate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 57. In certain embodiments, a VL sequence having at least 90%, 91%, 92%, 93%, 94%, 95%, 96%, 97%, 98%, or 99% identity to the amino acid sequence of SEQ ID NO: 57 contains substitutions (e.g., conservative substitutions), insertions, or deletions relative to the reference sequence, but an anti-CD79b antibody comprising that sequence retains the ability to bind to CD79b. In certain embodiments, a total of 1 to 10 amino acids have been substituted, inserted and/or deleted in SEQ ID NO: 57. In certain embodiments, a total of 1 to 5 amino acids have been substituted, inserted and/or deleted in SEQ ID NO: 57. In certain embodiments, the substitutions, insertions, or deletions occur in regions outside the HVRs (i.e., in the FRs). Optionally, the anti-CD79b antibody comprises the VL sequence of SEQ ID NO: 57, including post-translational modifications of that sequence. In a particular embodiment, the VL comprises one, two or three HVRs selected from (a) HVR-L1 comprising the amino acid sequence of SEQ ID NO: 61; (b) HVR-L2 comprising the amino acid sequence of SEQ ID NO: 62; and (c) HVR-L3 comprising the amino acid sequence of SEQ ID NO: 63.

In another aspect, an antibody-drug conjugate comprising an anti-CD79b antibody is provided, wherein the antibody comprises a VH as in any of the embodiments provided above, and a VL as in any of the embodiments provided above.

In one embodiment, an antibody-drug conjugate is provided, wherein the antibody comprises the VH and VL sequences in SEQ ID NO: 56 and SEQ ID NO: 57, respectively, including post-translational modifications of those sequences.

In a further aspect, provided herein are antibody-drug conjugate comprising antibodies that bind to the same epitope as an anti-CD79b antibody provided herein. For example, in certain embodiments, an immunoconjugate is provided comprising an antibody that binds to the same epitope as an anti-CD79b antibody comprising a VH sequence of SEQ ID NO: 56 and a VL sequence of SEQ ID NO: 57, respectively.

In a further aspect of the invention, an anti-CD79b antibody of an antibody-drug conjugate according to any of the above embodiments is a monoclonal antibody, including a human antibody. In one embodiment, an anti-CD79b antibody of an antibody-drug conjugate is an antibody fragment, e.g., a Fv, Fab, Fab′, scFv, diabody, or F(ab′)₂ fragment. In another embodiment, the antibody is a substantially full length antibody, e.g., an IgG1 antibody, IgG2a antibody or other antibody class or isotype as defined herein.

Table of CD79b Antibody Sequences

| SEQ ID NO | Description | Sequence |
|---|---|---|
| 56 | anti-CD₇₉b | EVQLVESGGG LVQPGGSLRL SCAASGYTFS SYWIEWVRQA |
| | huMA79bv28 | PGKGLEWIGE ILPGGGDTNY NEIFKGRATF SADTSKNTAY |
| | heavy chain | LQMNSLRAED TAVYYCTRRV PIRLDYWGQG TLVTVSS |
| | variable region | |
| 57 | anti-CD₇₉b | DIQLTQSPSS LSASVGDRVT ITCKASQSVD YEGDSFLNWY |
| | huMA79bv28 | QQKPGKAPKL LIYAASNLES GVPSRFSGSG SGTDFTLTIS |
| | light chain | SLQPEDFATY YCQQSNEDPL TFGQGTKVEI KR |
| | variable region | |
| 58 | anti-CD₇₉b | GYTFSSYWIE |
| | huMA79bv28 | |
| | HVR-H₁ | |
| 59 | anti-CD₇₉b | GEILPGGGDTNYNEIFKG |
| | huMA79bv28 | |
| | HVR-H₂ | |
| 60 | anti-CD₇₉b | TRRVPIRLDY |
| | huMA79bv28 | |
| | HVR-H₃ | |
| 61 | anti-CD₇₉b | KASQSVDYEGDSFLN |
| | huMA79bv28 | |
| | HVR-L₁ | |
| 62 | anti-CD₇₉b | AASNLES |
| | huMA79bv28 | |
| | HVR-L₂ | |
| 63 | anti-CD₇₉b | QQSNEDPLT |
| | huMA79bv28 | |
| | HVR-L₃ | |

Human HER2 Precursor Protein

Details of an exemplary human HER2 precursor protein with signal sequences is provided below

| SEQ ID NO | Description | Sequence |
|---|---|---|
| 64 | Exemplary human | MELAALCRWG LLLALLPPGA ASTQVCTGTD MKLRLPASPE |
| | HER2 precursor | THLDMLRHLY QGCQVVQGNL ELTYLPTNAS LSFLQDIQEV |
| | protein, with | QGYVLIAHNQ VRQVPLQRLR IVRGTQLFED NYALAVLDNG |
| | signal sequence | DPLNNTTPVT GASPGGLREL QLRSLTEILK GGVLIQRNPQ |
| | | LCYQDTILWK DIFHKNNQLA LTLIDTNRSR ACHPCSPMCK |
| | | GSRCWGESSE DCQSLTRTVC AGGCARCKGP LPTDCCHEQC |
| | | AAGCTGPKHS DCLACLHFNH SGICELHCPA LVTYNTDTFE |
| | | SMPNPEGRYT FGASCVTACP YNYLSTDVGS CTLVCPLHNQ |
| | | EVTAEDGTQR CEKCSKPCAR VCYGLGMEHL REVRAVTSAN |
| | | IQEFAGCKKI FGSLAFLPES FDGDPASNTA PLQPEQLQVF |
| | | ETLEEITGYL YISAWPDSLP DLSVFQNLQV IRGRILHNGA |
| | | YSLTLQGLGI SWLGLRSLRE LGSGLALIHH NTHLCFVHTV |
| | | PWDQLFRNPH QALLHTANRP EDECVGEGLA CHQLCARGHC |
| | | WGPGPTQCVN CSQFLRGQEC VEECRVLQGL PREYVNARHC |
| | | LPCHPECQPQ NGSVTCFGPE ADQCVACAHY KDPPFCVARC |
| | | PSGVKPDLSY MPIWKFPDEE GACQPCPINC THSCVDLDDK |
| | | GCPAEQRASP LTSIISAVVG ILLVVVLGVV FGILIKRRQQ |
| | | KIRKYTMRRL LQETELVEPL TPSGAMPNQA QMRILKETEL |
| | | RKVKVLGSGA FGTVYKGIWI PDGENVKIPV AIKVLRENTS |
| | | PKANKEILDE AYVMAGVGSP YVSRLLGICL TSTVQLVTQL |
| | | MPYGCLLDHV RENRGRLGSQ DLLNWCMQIA KGMSYLEDVR |
| | | LVHRDLAARN VLVKSPNHVK ITDFGLARLL DIDETEYHAD |
| | | GGKVPIKWMA LESILRRRFT HQSDVWSYGV TVWELMTFGA |
| | | KPYDGIPARE IPDLLEKGER LPQPPICTID VYMIMFVKCWM |
| | | IDSECRPRFR ELVSEFSRMA RDPQRFVVIQ NEDLGPASPL |
| | | DSTFYRSLLE DDDMGDLVDA EEYLVPQQGF FCPDPAPGAG |
| | | GMVHHRHRSS STRSGGGDLT LGLEPSEEEA PRSPLAPSEG |
| | | AGSDVFDGDL GMGAAKGLQS LPTHDPSPLQ RYSEDPTVPL |
| | | PSETDGYVAP LTCSPQPEYV NQPDVRPQPP SPREGPLPAA |
| | | RPAGATLERP KTLSPGKNGV VKDVFAFGGA VENPEYLTPQ |
| | | GGAAPQPHPP PAFSPAFDNL YYWDQDPPER GAPPSTFKGT |
| | | PTAENPEYLG LDVPV |

Antibody Affinity

In certain embodiments, an antibody provided herein has a dissociation constant (Kd) of ≤1 μM, ≤100 nM, ≤50 nM, ≤10 nm, ≤5 nM, ≤1 nM, ≤0.1 nM, ≤0.01 nM, or ≤0.001 nM, and optionally is ≥10⁻¹³ M. (e.g. 10⁻⁸ M or less, e.g. from 10⁻⁸ M to 10⁻¹³ M, e.g., from 10⁻⁹ M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCF T-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, NJ) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethyl-aminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (−0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ, See, e.g., Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instalments) or a 8000-series SLM-AMINCO spectrophotometer (ThermoSpectronic) with a stirred cuvette.

Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9: 129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9: 129-134 (2003); and Hollinger et al., Proc. Natl. Acad. Sci. USA 90: 6444-6448 (1993). Triabodies and tetrabodies are also described in Hudson et al., Nat. Med. 9: 129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516).

Antibody fragments can be made by various techniques, including but not limited to proteolytic digestion of an intact antibody as well as production by recombinant host cells (e.g. E. coli or phage), as described herein.

Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13: 1619-1633 (2008), and are further described, e.g., in Riechmann et al., Nature 332:323-329 (1988); Queen et al., Proc. Nat'l Acad. Sci. USA 86: 10029-10033 (1989); U.S. Pat. Nos. 5,821,337, 7,527,791, 6,982,321, and 7,087,409; Kashmiri et al, Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol, 151:2623 (1993)); human mature (somatically mutated) framework regions or human germline framework regions (see, e.g., Almagro and Fransson, Front. Biosci. 13: 1619-1633 (2008)); and framework regions derived from screening FR libraries (see, e.g., Baca et al., J. Biol. Chem. 272: 10678-10684 (1997) and Rosok et al., J. Biol. Chem. 271:22611-22618 (1996)).

Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23: 1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol, 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in L1 et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, XiandaiMianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. Methods in Molecular Biology 178: 1-37 (O'Brien et al., ed., Human Press, Totowa, N J, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, Methods in Molecular Biology 248: 161-175 (Lo, ed., Human Press, Totowa, N J, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol, 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol, 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, bispecific antibodies may bind to two different epitopes of the same target. Bispecific antibodies may also be used to localize cytotoxic agents to cells which express the target. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). The term “knob-into-hole” or “KnH” technology as used herein refers to the technology directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc binding interfaces, CL:CH1 interfaces or VH/VL interfaces of antibodies (see, e.g., US 2011/0287009, US2007/0178552, WO 96/027011, WO 98/050431, Zhu et al., 1997, Protein Science 6:781-788, and WO2012/106587). In some embodiments, KnHs drive the pairing of two different heavy chains together during the manufacture of multispecific antibodies.

For example, multispecific antibodies having KnH in their Fc regions can further comprise single variable domains linked to each Fc region, or further comprise different heavy chain variable domains that pair with similar or different light chain variable domains. KnH technology can be also be used to pair two different receptor extracellular domains together or any other polypeptide sequences that comprises different target recognition sequences (e.g., including affibodies, peptibodies and other Fc fusions).

The term “knob mutation” as used herein refers to a mutation that introduces a protuberance (knob) into a polypeptide at an interface in which the polypeptide interacts with another polypeptide. In some embodiments, the other polypeptide has a hole mutation.

The term “hole mutation” as used herein refers to a mutation that introduces a cavity (hole) into a polypeptide at an interface in which the polypeptide interacts with another polypeptide. In some embodiments, the other polypeptide has a knob mutation.

A brief nonlimiting discussion is provided below.

A “protuberance” refers to at least one amino acid side chain which projects from the interface of a first polypeptide and is therefore positionable in a compensatory cavity in the adjacent interface (i.e. the interface of a second polypeptide) so as to stabilize the heteromultimer, and thereby favor heteromultimer formation over homomultimer formation, for example. The protuberance may exist in the original interface or may be introduced synthetically (e.g., by altering nucleic acid encoding the interface). In some embodiments, nucleic acid encoding the interface of the first polypeptide is altered to encode the protuberance. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The side chain volumes of the various amino residues are shown, for example, in Table 1 of US2011/0287009. A mutation to introduce a “protuberance” may be referred to as a “knob mutation.”

In some embodiments, import residues for the formation of a protuberance are naturally occurring amino acid residues selected from arginine (R), phenylalanine (F), tyrosine (Y) and tryptophan (W). In some embodiments, an import residue is tryptophan or tyrosine. In some embodiment, the original residue for the formation of the protuberance has a small side chain volume, such as alanine, asparagine, aspartic acid, glycine, serine, threonine or valine.

A “cavity” refers to at least one amino acid side chain which is recessed from the interface of a second polypeptide and therefore accommodates a corresponding protuberance on the adjacent interface of a first polypeptide. The cavity may exist in the original interface or may be introduced synthetically (e.g. by altering nucleic acid encoding the interface). In some embodiments, nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. In some embodiments, import residues for the formation of a cavity are naturally occurring amino acid residues selected from alanine (A), serine (S), threonine (T) and valine (V). In some embodiments, an import residue is serine, alanine or threonine. In some embodiments, the original residue for the formation of the cavity has a large side chain volume, such as tyrosine, arginine, phenylalanine or tryptophan. A mutation to introduce a “cavity” may be referred to as a “hole mutation.”

The protuberance is “positionable” in the cavity which means that the spatial location of the protuberance and cavity on the interface of a first polypeptide and second polypeptide respectively and the sizes of the protuberance and cavity are such that the protuberance can be located in the cavity without significantly perturbing the normal association of the first and second polypeptides at the interface. Since protuberances such as Tyr, Phe and Trp do not typically extend perpendicularly from the axis of the interface and have preferred conformations, the alignment of a protuberance with a corresponding cavity may, in some instances, rely on modeling the protuberance/cavity pair based upon a three-dimensional structure such as that obtained by X-ray crystallography or nuclear magnetic resonance (NMR). This can be achieved using widely accepted techniques in the art.

In some embodiments, a knob mutation in an IgG1 constant region is T366W (EU numbering). In some embodiments, a hole mutation in an IgG1 constant region comprises one or more mutations selected from T366S, L368A and Y407V (EU numbering). In some embodiments, a hole mutation in an IgG1 constant region comprises T366S, L368A and Y407V (EU numbering).

In some embodiments, a knob mutation in an IgG4 constant region is T366W (EU numbering). In some embodiments, a hole mutation in an IgG4 constant region comprises one or more mutations selected from T366S, L368A, and Y⁴⁰⁷V (EU numbering). In some embodiments, a hole mutation in an IgG4 constant region comprises T366S, L368A, and Y⁴⁰⁷V (EU numbering).

Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol, 148(5): 1547-1553 (1992)); using “diabody” technology for making bispecific antibody fragments (see, e.g., Hollinger et al., Proc. Natl Acad. Sci. USA, 90:6444-6448 (1993)); and using single-chain Fv (sFv) dimers (see, e.g. Gruber et al., J. Immunol, 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to the target as well as another, different antigen (see, US 2008/0069820, for example).

Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Substitution, Insertion, and Deletion Variants

In certain embodiments, antibody variants having one or more amino acid substitutions are provided. Sites of interest for substitutional mutagenesis include the HVRs and FRs. Conservative substitutions are shown below in a Table of conservative substitutions under the heading of “preferred substitutions.” More substantial changes are provided in the Table under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

Table of conservative substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207: 179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178: 1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244: 1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH₂ domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%). The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108; US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336: 1239-1249 (2004); Yamane-Ohnuki et al. Biotech. Bioeng. 87: 614 (2004). Examples of cell lines capable of producing defucosylated antibodies include Led 3 CHO cells deficient in protein fucosylation (Ripka et al. Arch. Biochem. Biophys. 249:533-545 (1986); US Pat Appl No US 2003/0157108; and WO 2004/056312, especially at Example 11), and knockout cell lines, such as alpha-1,6-fucosyl transferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

Fc Region Variants

In certain embodiments, one or more amino acid modifications may be introduced into the Fc region of an antibody provided herein, thereby generating an Fc region variant.

The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82: 1499-1502 (1985); 5,821,337 (see Bruggemann, M. et al., J. Exp. Med. 166: 1351-1361 (1987)).

Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202: 163 (1996); Cragg, M. S. et al., Blood 101: 1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12): 1759-1769 (2006)).

In some embodiments, one or more amino acid modifications may be introduced into the Fc portion of the antibody provided herein in order to increase IgG binding to the neonatal Fc receptor. In certain embodiments, the antibody comprises the following three mutations according to EU numbering: M252Y, S254T, and T256E (the “YTE mutation”) (U.S. Pat. No. 8,697,650; see also Dall'Acqua et al., Journal of Biological Chemistry 281(33):23514-23524 (2006). In certain embodiments, the YTE mutation does not affect the ability of the antibody to bind to its cognate antigen. In certain embodiments, the YTE mutation increases the antibody's serum half-life compared to the native (i.e., non-YTE mutant) antibody. In some embodiments, the YTE mutation increases the serum half-life of the antibody by 3-fold compared to the native (i.e., non-YTE mutant) antibody. In some embodiments, the YTE mutation increases the serum half-life of the antibody by 2-fold compared to the native (i.e., non-YTE mutant) antibody. In some embodiments, the YTE mutation increases the serum half-life of the antibody by 4-fold compared to the native (i.e., non-YTE mutant) antibody. In some embodiments, the YTE mutation increases the serum half-life of the antibody by at least 5-fold compared to the native (i.e., non-YTE mutant) antibody. In some embodiments, the YTE mutation increases the serum half-life of the antibody by at least 10-fold compared to the native (i.e., non-YTE mutant) antibody. See, e.g., U.S. Pat. No. 8,697,650; see also Dall'Acqua et al., Journal of Biological Chemistry 281(33):23514-23524 (2006).

In certain embodiments, the YTE mutant provides a means to modulate antibody-dependent cell-mediated cytotoxicity (ADCC) activity of the antibody. In certain embodiments, the YTEO mutant provides a means to modulate ADCC activity of a humanized IgG antibody directed against a human antigen. See, e.g., U.S. Pat. No. 8,697,650; see also Dall'Acqua et al., Journal of Biological Chemistry 281(33):23514-23524 (2006).

In certain embodiments, the YTE mutant allows the simultaneous modulation of serum half-life, tissue distribution, and antibody activity (e.g., the ADCC activity of an IgG antibody). See, e.g., U.S. Pat. No. 8,697,650; see also Dall'Acqua et al., Journal of Biological Chemistry 281(33):23514-23524 (2006).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fe mutant with substitution of residues 265 and 297 to alanine (U.S. Pat. No. 7,332,581).

In certain embodiments, the proline at position 329 (EU numbering) (P329) of a wild-type human Fc region is substituted with glycine or arginine or an amino acid residue large enough to destroy the proline sandwich within the Fc/Fc gamma receptor interface, that is formed between the P329 of the Fe and tryptophane residues W87 and W110 of FcgRIII (Sondermann et al., Nature 406, 267-273 (20 Jul. 2000)). In a further embodiment, at least one further amino acid substitution in the Fc variant is S228P, E233P, L234A, L235A, L235E, N297A, N297D, or P331S and still in another embodiment said at least one further amino acid substitution is L234A and L235A of the human IgG1 Fc region or S228P and L235E of the human IgG4 Fc region, all according to EU numbering (U.S. Pat. No. 8,969,526 which is incorporated by reference in its entirety).

In certain embodiments, a polypeptide comprises the Fc variant of a wild-type human IgG Fc region wherein the polypeptide has P329 of the human IgG Fc region substituted with glycine and wherein the Fc variant comprises at least two further amino acid substitutions at L234A and L235A of the human IgG1 Fc region or S228P and L235E of the human IgG4 Fc region, and wherein the residues are numbered according to the EU numbering (U.S. Pat. No. 8,969,526 which is incorporated by reference in its entirety). In certain embodiments, the polypeptide comprising the P329G, L234A and L235A (EU numbering) substitutions exhibit a reduced affinity to the human FcγRIIIA and FcγRIIA, for down-modulation of ADCC to at least 20% of the ADCC induced by the polypeptide comprising the wildtype human IgG Fc region, and/or for down-modulation of ADCP (U.S. Pat. No. 8,969,526 which is incorporated by reference in its entirety).

In a specific embodiment the polypeptide comprising an Fc variant of a wildtype human Fc polypeptide comprises a triple mutation: an amino acid substitution at position Pro329, a L234A and a L235A mutation according to EU numbering (P329/LALA) (U.S. Pat. No. 8,969,526 which is incorporated by reference in its entirety). In specific embodiments, the polypeptide comprises the following amino acid substitutions: P329G, L234A, and L235A according to EU numbering.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

Cysteine Engineered Antibody Variants

In certain embodiments, it may be desirable to create cysteine engineered antibodies, e.g., a “THIOMAB™” or TDC, in which one or more residues of an antibody are substituted with cysteine residues. In particular embodiments, the substituted residues occur at sites of the antibody that are available for conjugation. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: K149 (Kabat numbering) of the light chain; V205 (Kabat numbering) of the light chain; A118 (EU numbering) of the heavy chain; A140 (EU numbering) of the heavy chain; L174 (EU numbering) of the heavy chain; Y373 (EU numbering) of the heavy chain; and S400 (EU numbering) of the heavy chain Fc region. In specific embodiments, the antibodies described herein comprise the HC-A140C (EU numbering) cysteine substitution. In specific embodiments, the antibodies described herein comprise the LC-K149C (Kabat numbering) cysteine substitution. In specific embodiments, the antibodies described herein comprise the HC-A118C (EU numbering) cysteine substitution. Cysteine engineered antibodies may be generated as described, e.g., in U.S. Pat. No. 7,521,541.

In certain embodiments, the antibody comprises one of the following heavy chain cysteine substitutions:

| Chain | | EU Mutation | Kabat Mutation |
|---|---|---|---|
| (HC/LC) | Residue | Site # | Site # |
| HC | T | 114 | 110 |
| HC | A | 140 | 136 |
| HC | L | 174 | 170 |
| HC | L | 179 | 175 |
| HC | T | 187 | 183 |
| HC | T | 209 | 205 |
| HC | V | 262 | 258 |
| HC | G | 371 | 367 |
| HC | Y | 373 | 369 |
| HC | E | 382 | 378 |
| HC | S | 424 | 420 |
| HC | N | 434 | 430 |
| HC | Q | 438 | 434 |

In certain embodiments, the antibody comprises one of the following light chain cysteine substitutions:

| Chain | | EU Mutation | Kabat Mutation |
|---|---|---|---|
| (HC/LC) | Residue | Site # | Site # |
| LC | I | 106 | 106 |
| LC | R | 108 | 108 |
| LC | R | 142 | 142 |
| LC | K | 149 | 149 |
| LC | V | 205 | 205 |

A nonlimiting exemplary hu7C2.v2.2.LA light chain (LC) K149C THIOMAB™ has the heavy chain and light chain amino acid sequences of SEQ ID NOs: 26 and 30, respectively. A nonlimiting exemplary hu7C2.v2.2.LA heavy chain (HC) A118C THIOMAB™ has the heavy chain and light chain amino acid sequences of SEQ ID NOs: 31 and 25, respectively.

Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

Recombinant Methods and Compositions

Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0 Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fe effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NT, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22: 1409-1414 (2004), and L1 et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977); baby hamster kidney cells (BHK); mouse Sertoli cells (TM4 cells as described, e.g., in Mather, Biol. Reprod. 23:243-251 (1980); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

Administration & Dose

Compounds of formula I may be administered alone or in combination with one or another or with one or more pharmacologically active compounds which are different from the compounds of formula I.

Compounds of the invention may suitably be combined with various components to produce compositions of the invention. Suitably the compositions are combined with a pharmaceutically acceptable carrier or diluent to produce a pharmaceutical composition (which may be for human or animal use). Suitable carriers and diluents include isotonic saline solutions, for example phosphate-buffered saline. Useful pharmaceutical compositions and methods for their preparation may be found in standard pharmaceutical texts. See, for example, Handbook for Pharmaceutical Additives, 3rd Edition (eds. M. Ash and I. Ash), 2007 (Synapse Information Resources, Inc., Endicott, New York, USA) and Remington: The Science and Practice of Pharmacy, 21st Edition (ed. D. B. Troy) 2006 (Lippincott, Williams and Wilkins, Philadelphia, USA) which are incorporated herein by reference.

The compounds of the invention may be administered by any suitable route. Suitably the compounds of the invention will normally be administered orally or by any parenteral route, in the form of pharmaceutical preparations comprising the active ingredient, optionally in the form of a non-toxic organic, or inorganic, acid, or base, addition salt, in a pharmaceutically acceptable dosage form.

The compounds of the invention, their pharmaceutically acceptable salts, and pharmaceutically acceptable solvates of either entity can be administered alone but will generally be administered in admixture with a suitable pharmaceutical excipient diluent or carrier selected with regard to the intended route of administration and standard pharmaceutical practice.

For example, the compounds of the invention or salts or solvates thereof can be administered orally, buccally or sublingually in the form of tablets, capsules (including soft gel capsules), ovules, elixirs, solutions or suspensions, which may contain flavouring or colouring agents, for immediate-, delayed-, modified-, sustained-, controlled-release or pulsatile delivery applications. The compounds of the invention may also be administered via fast dispersing or fast dissolving dosages forms.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethyl cellulose (HPMC), hydroxypropylcellulose (HPC), sucrose, gelatin and acacia. Additionally, lubricating agents such as magnesium stearate, stearic acid, glyceryl behenate and tale may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients maybe present both within the dosage form i.e. within the matrix, and/or on the dosage form i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol.

The compounds of the invention can also be administered parenterally, for example, intravenously, intra-arterially, or they may be administered by infusion techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Suitably formulation of the invention is optimised for the route of administration e.g. oral, intravenously, etc.

Administration may be in one dose, continuously or intermittently (e.g. in divided doses at appropriate intervals) during the course of treatment. Methods of determining the most effective means and dosage are well known to a skilled person and will vary with the formulation used for therapy, the purpose of the therapy, the target cell(s) being treated, and the subject being treated. Single or multiple administrations can be carried out with the dose level and the dose regimen being selected by the treating physician, veterinarian, or clinician.

Depending upon the disorder and patient to be treated, as well as the route of administration, the compositions may be administered at varying doses. For example, a typical dosage for an adult human may be 100 ng to 25 mg (suitably about 1 micro g to about 10 mg) per kg body weight of the subject per day.

Suitably guidance may be taken from studies in test animals when estimating an initial dose for human subjects. For example when a particular dose is identified for mice, suitably an initial test dose for humans may be approx. 0.5× to 2× the mg/Kg value given to mice.

Other Forms

Unless otherwise specified, included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

Isomers, Salts and Solvates

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; alpha- and beta-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH.

A reference to a class of structures may well include structurally isomeric forms falling within that class (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not apply to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro. In some cases, the compounds of formula (I) can exist as tautomers. Suitably, the compounds of formula (I) include the keto-enol tautomers of the drawn structures.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof.

Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

Compounds of Formula (I), which include compounds specifically named above, may form pharmaceutically acceptable complexes, salts, solvates and hydrates. These salts include nontoxic acid addition salts (including di-acids) and base salts.

If the compound is cationic, or has a functional group which may be cationic (e.g. —NH₂ may be —NH₃⁺), then an acid addition salt may be formed with a suitable anion.

Examples of suitable inorganic anions include, but are not limited to, those derived from the following inorganic acids hydrochloric acid, nitric acid, nitrous acid, phosphoric acid, sulfuric acid, sulphurous acid, hydrobromic acid, hydroiodic acid, hydrofluoric acid, phosphoric acid and phosphorous acids. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfonate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate and xinofoate salts.

For example, if the compound is anionic, or has a functional group which may be anionic (e.g. —COOH may be —COO⁻), then a base salt may be formed with a suitable cation. Examples of suitable inorganic cations include, but are not limited to, metal cations, such as an alkali or alkaline earth metal cation, ammonium and substituted ammonium cations, as well as amines. Examples of suitable metal cations include sodium (Na⁺) potassium (K⁺), magnesium (Mg²⁺), calcium (Ca²⁺), zinc (Zn²⁺), and aluminum (Al³⁺). Examples of suitable organic cations include, but are not limited to, ammonium ion (i.e. NH⁴⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺. Examples of suitable amines include arginine, N,N′-dibenzylethylene-diamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine. For a discussion of useful acid addition and base salts, see S. M. Berge et al., J. Pharm. Sci. (1977) 66:1-19; see also Stahl and Wermuth, Handbook of Pharmaceutical Salts: Properties, Selection, and Use (2011)

Pharmaceutically acceptable salts may be prepared using various methods. For example, one may react a compound of Formula (I) with an appropriate acid or base to give the desired salt. One may also react a precursor of the compound of Formula (I) with an acid or base to remove an acid- or base-labile protecting group or to open a lactone or lactam group of the precursor. Additionally, one may convert a salt of the compound of Formula (I) to another salt through treatment with an appropriate acid or base or through contact with an ion exchange resin. Following reaction, one may then isolate the salt by filtration if it precipitates from solution, or by evaporation to recover the salt. The degree of ionization of the salt may vary from completely ionized to almost non-ionized.

It may be convenient or desirable to prepare, purify, and/or handle a corresponding solvate of the active compound. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent may be isotopically substituted (e.g., D₂O, acetone-d6, DMSO-d6).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions.v In such cases, non-stoichiometry will typically be observed.

Compounds of formula (I) include imine, carbinolamine and carbinolamine ether forms of the PBD or PDD. The carbinolamine or the carbinolamine ether is formed when a nucleophilic solvent (H₂O, ROH) adds across the imine bond of the PBD or PDD moiety. The balance of these equilibria between these forms depend on the conditions in which the compounds are found, as well as the nature of the moiety itself.

These compounds may be isolated in solid form, for example, by lyophilisation.

Further particular and preferred aspects are set out in the accompanying independent and dependent claims. Features of the dependent claims may be combined with features of the independent claims as appropriate, and in combinations other than those explicitly set out in the claims.

### Synthetic Strategies

The compounds of Formula (I) may be prepared using the techniques described below. Some of the schemes and examples may omit details of common reactions, including oxidations, reductions, and so on, separation techniques (extraction, evaporation, precipitation, chromatography, filtration, trituration, crystallization, and the like), and analytical procedures, which are known to persons of ordinary skill in the art of organic chemistry. The details of such reactions and techniques can be found in a number of treatises, including Richard Larock, Comprehensive Organic Transformations, A Guide to Functional Group Preparations, 2nd Ed (2010), and the multi-volume series edited by Michael B. Smith and others, Compendium of Organic Synthetic Methods (1974 et seq.). Starting materials and reagents may be obtained from commercial sources or may be prepared using literature methods. Some of the reaction schemes may omit minor products resulting from chemical transformations (e.g., an alcohol from the hydrolysis of an ester, CO₂ from the decarboxylation of a diacid, etc.). In addition, in some instances, reaction intermediates may be used in subsequent steps without isolation or purification (i.e., in situ).

In some of the reaction schemes and examples below, certain compounds can be prepared using protecting groups, which prevent undesirable chemical reaction at otherwise reactive sites. Protecting groups may also be used to enhance solubility or otherwise modify physical properties of a compound. For a discussion of protecting group strategies, a description of materials and methods for installing and removing protecting groups, and a compilation of useful protecting groups for common functional groups, including amines, carboxylic acids, alcohols, ketones, aldehydes, and so on, see T. W. Greene and P. G. Wuts, Protecting Groups in Organic Chemistry, 4th Edition, (2006) and P. Kocienski, Protective Groups, 3rd Edition (2005).

Generally, the chemical transformations described throughout the specification may be carried out using substantially stoichiometric amounts of reactants, though certain reactions may benefit from using an excess of one or more of the reactants.

Additionally, many of the reactions disclosed throughout the specification may be carried out at about room temperature (RT) and ambient pressure, but depending on reaction kinetics, yields, and so on, some reactions may be run at elevated pressures or employ higher temperatures (e.g., reflux conditions) or lower temperatures (e.g., −78° C. to 0° C.). Any reference in the disclosure to a stoichiometric range, a temperature range, a pH range, etc., whether or not expressly using the word “range,” also includes the indicated endpoints.

Many of the chemical transformations may also employ one or more compatible solvents, which may influence the reaction rate and yield. Depending on the nature of the reactants, the one or more solvents may be polar protic solvents (including water), polar aprotic solvents, non-polar solvents, or some combination. Representative solvents include saturated aliphatic hydrocarbons (e.g., n-pentane, n-hexane, n-heptane, n-octane); aromatic hydrocarbons (e.g., benzene, toluene, xylenes); halogenated hydrocarbons (e.g., methylene chloride, chloroform, carbon tetrachloride); aliphatic alcohols (e.g., methanol, ethanol, propan-1-ol, propan-2-ol, butan-1-ol, 2-methyl-propan-1-ol, butan-2-ol, 2-methyl-propan-2-ol, pentan-1-ol, 3-methyl-butan-1-ol, hexan-1-ol, 2-methoxy-ethanol, 2-ethoxy-ethanol, 2-butoxy-ethanol, 2-(2-methoxy-ethoxy)-ethanol, 2-(2-ethoxy-ethoxy)-ethanol, 2-(2-butoxy-ethoxy)-ethanol); ethers (e.g., diethyl ether, di-isopropyl ether, dibutyl ether, 1,2-dimethoxy-ethane, 1,2-diethoxy-ethane, 1-methoxy-2-(2-methoxy-ethoxy)-ethane, 1-ethoxy-2-(2-ethoxy-ethoxy)-ethane, tetrahydrofuran, 1,4-dioxane); ketones (e.g., acetone, methyl ethyl ketone); esters (methyl acetate, ethyl acetate); nitrogen-containing solvents (e.g., formamide, N,N-dimethylformamide, acetonitrile, N-methyl-pyrrolidone, pyridine, quinoline, nitrobenzene); sulfur-containing solvents (e.g., carbon disulfide, dimethyl sulfoxide, tetrahydro-thiophene-1,1,-dioxide); and phosphorus-containing solvents (e.g., hexamethylphosphoric triamide).

Further particular and preferred aspects are set out in the accompanying independent and dependent claims. Features of the dependent claims may be combined with features of the independent claims as appropriate, and in combinations other than those explicitly set out in the claims.

### BRIEF DESCRIPTION OF THE DRAWINGS

Embodiments of the present invention will now be described further, with reference to the accompanying drawings, in which:

FIG. 1 shows a snapshot of a Molecular Dynamics Simulation showing cross-linking of a PBD-Phenyl-CBI covalently bound in the intrastrand mode in the four base-pair sequence 5′-XC(G)AAT(A)X-3′, showing excellent accommodation in the minor groove with little distortion of the central base-pairing.

FIG. 2 shows a snapshot of a Molecular Dynamics Simulation showing cross-linking of a PBD-Phenyl-CBI covalently bound in the interstrand mode in the five base-pair sequence 5′-XC(G)ATTAX-3′, showing excellent accommodation in the minor groove.

FIG. 3 shows a snapshot of a Molecular Dynamics Simulation showing 27eS (21) covalently bound in the intrastrand mode in the four base-pair sequence 5′-XC(G)TTT(A)X-3′, showing excellent accommodation in the minor groove with little distortion of the central base-pairing.

FIG. 4 shows a snapshot of a Molecular Dynamics Simulation showing 27eS covalently bound in the interstrand mode in the five base-pair sequence 5′-XC(G)ATTAX-3′, showing excellent accommodation in the minor groove with little distortion of the central base-pairing.

FIG. 5 shows a snapshot of a Molecular Dynamics Simulation showing a C1-linked PBD-Phenyl-CBI dual-covalently bound to the minor groove. PBD-Phenyl-CBI covalently bound in the interstrand mode in the four base-pair sequence 5′-XC(G)ATAX-3′, showing excellent accommodation in the minor groove with little distortion of the central base-pairing.

FIG. 6 shows a snapshot of a Molecular Dynamics Simulation illustrating PBD-CBI forming an interstrand cross-link in the minor groove of DNA across the sequence 5′-XC(G)ATTAX-3′. PBD-CBI shows little distortion of the central base-pairing, suggesting excellent accommodation in the minor groove of DNA.

FIG. 7 shows a snapshot of a Molecular Dynamics Simulation illustrating PDD-CBI (C8-linked) forming an intrastrand cross-link in the minor groove of DNA across the sequence 5′-XC(G)ATT(A)X-3′. The PDD-CBI shows little distortion of the central base-pairing, suggesting excellent accommodation in the minor groove of DNA.

FIG. 8 shows a sequence of the labelled strand of the TyrT DNA fragment used in the study.

FIG. 9 shows an autoradiograph of a denaturing polyacrylamide gel showing DNA interstrand cross-linking by 13 in linear ³²P-end-labelled TyrT DNA following overnight incubation at 37° C. at various concentrations. FA=formamide.

FIG. 10 shows an autoradiograph of a denaturing polyacrylamide gel showing DNA interstrand cross-linking by the PBD dimer Talirine in linear ³²P-end-labelled TyrT DNA following overnight incubation at 37° C. at various concentrations.

FIG. 11 shows a cleavage pattern showing the interaction of 13 with TyrT DNA fragment. Ligand concentrations are shown at the top of the gel. Tracks labelled “GA” are markers for specific purines.

FIG. 12 shows a sequence of the TyrT DNA fragment showing the possible cross-linked adducts relating to the observed cleavage sites on the electrophoresis gel produced by compound 13 due to thermally-induced cleavage at the sites of adnenine alkylation by the CBI unit.

FIG. 13 shows fluorescently labelled DNA duplexes used in the FRET melting study to study the formation of interstrand (top) and intrastrand (bottom) cross-links. The labels were fluorescein (F) and dabcyl (Q).

FIG. 14 shows FRET denaturation data for the two DNA sequences shown in FIG. 13. The melting temperature of each duplex increases significantly in proportion to the concentration of 13 present, providing strong supporting evidence that the compound can produce interstrand (top panel) and intrastrand (bottom panel) cross-links.

FIG. 15 shows a sequence of a labelled strand of the HexARev DNA fragment used in a biophysical characterisation study.

FIG. 16 shows a HexARev DNA fragment cleavage assay gel. Tracks labelled “GA” are markers for specific purines and “C” is a control, and cleavage points are represented by arrows. Starting from the left, the first set of tracks labelled 1, 2, 3, 4, 5, 6 and 7 represent compounds 13, 42, 59, 99, a control, talirine and a G-alkylator control respectively at a ligand concentration of 10 μM, the second (right hand) set of tracks labelled 1, 2, 3, 4, 5, 6 and 7 represent the same components at a ligand concentration of 100 nM.

FIG. 17 shows fluorescently labelled DNA duplexes used in the FRET melting study of further compounds (such as compound 42) to study the formation of interstrand (top and middle) and intrastrand (bottom) cross-links. The labels were fluorescein (F) and dabcyl (Q).

FIG. 18 shows FRET Denaturation data for compound 42 against each of the three DNA sequences (A—top sequence; B—middle sequence; and C—bottom sequence) shown in FIG. 17 at different concentrations with a control of the respective DNA sequence.

FIG. 19 FRET Denaturation data for compound 59 when incubated with the three DNA sequences shown in FIG. 17 (A—top sequence; B—middle sequence; and C—bottom sequence) at different concentrations with a control of the respective DNA sequence. The melting temperature of does not increase in proportion to the concentration of 59 present in any of the three sequences, suggesting that the carbamate moiety on the A-alkylating unit interferes with DNA binding.

FIG. 20 FRET Denaturation data for compound 152 when incubated with the three DNA sequences shown in FIG. 17 (A—top sequence; B—middle sequence; and C—bottom sequence) at different concentrations with a control of the respective DNA sequence. The melting temperature has a limited increase in proportion to the concentration of 152 present in all three sequences, suggesting that there is a limited level of DNA stabilisation.

FIG. 21 FRET Denaturation data for compound 149 when incubated with the three DNA sequences shown in FIG. 17 (A—top sequence; B—middle sequence; and C—bottom sequence) at different concentrations with a control of the respective DNA sequence. The melting temperature has a limited increase in proportion to the concentration of 149 present in all three sequences, suggesting that there is a limited level of DNA stabilisation.

FIG. 22 FRET Denaturation data for compound 83 (A—top sequence; B—middle sequence; and C—bottom sequence) when incubated with the three DNA sequences shown in FIG. 17 (A—top sequence; B—middle sequence; and C—bottom sequence) at different concentrations with a control of the respective DNA sequence. The melting temperatures do not increase when 83 is added to the DNA sequences, suggesting 83 causes a very limited degree of DNA stabilisation.

FIG. 23 FRET Denaturation data for compound 150 (A—top sequence; B—middle sequence; and C—bottom sequence) when incubated with the three DNA sequences shown in FIG. 17 at different concentrations with a control of the respective DNA sequence. The melting temperatures do not increase when 150 is added to the DNA sequences, suggesting 150 causes a very limited degree of DNA stabilisation.

FIG. 24 SEC profile of Antibody X. 98.9% monomer, 1.0% dimer, and 0.1% LMW as indicated. The peak at about 23 minutes originates from the formulation of the antibody FIG. 25 HIC profile of Antibody X.

FIG. 26 PLRP trace of Antibody X. Heavy (Ho) and light (Lo) chain peaks as indicated.

FIG. 27 HIC profile of IgG1-165. Average DAR calculated as 2.2 with the DAR species assigned starting with DAR 0.

FIG. 28 HIC profile of IgG1-171. Average DAR calculated as 1.9 with the DAR species assigned starting with DAR 0.

FIG. 29 SEC profile of IgG1-165; 96.7% monomer, 1.9% dimer, 1.4% HMW as indicated.

FIG. 30 Free toxin linker traces of the IgG1-165 sample. No free toxin linker could be detected in the ADC trace. Red: 5 pmol 165. Blue: IgG1-165 after protein precipitation; the identified peaks show residual proteinaceous material.

### EXAMPLES

### General Remarks

Unless otherwise stated, all reagents and synthetic building blocks and reagents were purchased from standard commercial suppliers, such as Maybridge Chemicals (UK), Fluorochem (USA), ChemShuttle Inc (USA), Merck KGaA, (Germany), VWR Ltd., Avantor Inc., (USA), Fischer Scientific, Inc. (USA), and Sigma-Aldrich (UK) and used as purchased. 3-(Bromomethyl)-benzeneacetic acid methyl ester was purchased from Beta Pharma Scientific Inc. (USA). Methyl 3-(bromomethyl)-1-benzothiophene-2-carboxylate was purchased from Enamine Ltd. (Ukraine). Methyl (2S)-piperidinecarboxylate hydrochloride and methyl 2-[6-(chloromethyl)-2-pyridyl]acetate hydrochloride were purchased from Apollo Scientific Ltd. (UK). Methyl 8-bromooctanoate was purchased from Combi-Blocks, Inc. (USA). (S)-(+)-2-Indolinemethanol was purchased from Carbosynth Ltd. (UK). N-Boc O-Bn-(S)-seco-CBIN-Boc O-Bn-(S)-seco-CBI, Alloc-Val-Ala-OH and Alloc-Val-Ala-PAB-PNP were purchased from YProTech (UK) and SYNthesis Med Chem (UK). Solvents were purchased from Sigma-Aldrich (UK) and Fisher Scientific (UK). Anhydrous reactions were carried out in pre-oven-dried glassware under an inert atmosphere of nitrogen or argon. Anhydrous solvents were used as purchased without further drying. Thin Layer Chromatography (TLC) was performed on silica gel aluminium plates (Merck 60, F₂₅₄), and flash column chromatography was carried out either manually, using silica gel (Merck 9385, 230-400 mesh ASTM, 40-63 PM) (whilst monitoring by thin layer chromatography: UV (254 nm) and an aqueous alkaline solution of potassium permanganate as stain), or using a Grace Reveleris® X2 automated Flash Chromatography System, or using a Biotage Isolera Dalton 2000 (automated mass-directed flash chromatography system). A11 Nuclear Magnetic Resonance (NMR) spectra were obtained at room temperature using a Bruker DPX400 or a Varian Mercury Vx Agilent 400 MHz spectrometer, for which chemical shifts are expressed in ppm relative to the solvent and coupling constants are expressed in Hz. Microwave reactions were carried out on an Anton Paar Monowave 300 microwave synthesis reactor, or a Biotage Initiator+microwave synthesizer. High Resolution Mass Spectrometry (HRMS) was performed on a Thermo Scientific-Exactive HCD Orbitrap Mass Spectrometer. Yields refer to isolated material (homogeneous by TLC or NMR) unless otherwise stated and names are assigned according to IUPAC nomenclature.

Liquid Chromatography Mass Spectroscopy (LCMS) analysis Methods A-C were performed on a Waters Alliance 2695 with water (A) and acetonitrile (B) comprising the mobile phases. Formic acid (0.1%) was added to both acetonitrile and water to ensure acidic conditions throughout the analysis. Function type: Diode array (535 scans). Column type: Monolithic C18 50×4.60 mm. Mass spectrometry data were collected using a Waters Micromass ZQ instrument coupled to a Waters 2695 HPLC with a Waters 2996 PDA. Waters Micromass ZQ parameters used were: Capillary (kV), 3.38; Cone (V), 35; Extractor (V), 3.0; Source temperature (° C.), 100; Desolvation Temperature (° C.), 200; Cone flow rate (L/h), 50; De-solvation flow rate (L/h), 250. LCMS gradient conditions are described as follows.

Method A (10 min): from 95% A/5% B to 50% B over 3 min. Then from 50% B to 80% B over 2 min. Then from 80% B to 95% B over 1.5 min and held constant for 1.5 min. This was then reduced to 5% B over 0.2 min and maintained to 5% B for 1.8 min. The flow rate was 0.5 mL/min, 200 μL was split via a zero dead volume T piece which passed into the mass spectrometer. The wavelength range of the UV detector was 220-400 nm.

Method B (5 min): from 95% A/5% B to 90% B over 3 min. Then from 90% B to 95% B over 0.5 min and held constant for 1 min. This was then reduced to 5% B over 0.5 min. The flow rate was 1.0 mL/min, 100 μL was split via a zero dead volume T piece which passed into the mass spectrometer. The wavelength range of the UV detector was 220-500 nm.

Method C (5 min): from 95% A/5% B, which was increased to 90% B over 3 min and to 95% B over a further 0.5 min. The gradient was then held at 95% B for 1 min and then returned to 5% B over 0.5 min. The total duration of the run was 5 minutes and the solvent flow rate was 1 mL/min, 100 μL was split via a zero dead volume T piece which passed into the mass spectrometer. The wavelength range of the UV detector was 220-500 nm.

Liquid Chromatography Mass Spectrometry (LCMS) analysis Methods D-G were performed on a Shimadzu LC-20AD series, Binary Pump, Diode Array Detector. Column type: Agilent Poroshell 120 EC-C18, 2.7 μm, 4.6×50 mm. Mobile phase: A: 0.05% formic acid in water (v/v); B: 0.05% formic acid in acetonitrile (v/v). Flow Rate: 1 mL/min at 25° C. Detector: 214 nm, 254 nm. Gradient stop time: 5 min. MS: 2020, Quadrupole LC/MS, Ion Source: API-ESI, TIC: 100-1300 m/z, Drying gas flow: 15 L/min, Nebulizer pressure: 1.5 L/min, Drying gas temperature: 250° C., Vcap: 4500V. Sample preparation: samples were dissolved in methanol at 1-10 μg/mL, then filtered through a 0.22 μm filter membrane. Injection volume: 1-10 μL. Gradient conditions are described as follows.

Method D (5 min): 20% A/80% B for 0.5 min, which was increased to 100% B over 3.5 min, then held at 100% B for 0.5 min. This was then returned to 20% A/80% B for 0.5 min.

Method E (5 min): 50% A/50% B for 0.5 min, which was increased to 100% B over 3.5 min, then held at 100% B for 0.5 min. This was then returned to 50% A/50% B for 0.5 min.

Method F (5 min): 85% A/15% B for 0.5 min, which was increased to 100% B over 3.5 min, then held at 100% B for 0.5 min. This was then returned to 85% A/15% B for 0.5 min.

Method G (5 min): 97% A/3% B for 0.5 min, which was increased to 30% A/70% B over 3.5 min, then to 100% B over 0.5 min. This was then returned to 97% A/3% B for 0.5 min.

Optical rotations were measured on a SGWzz-1 automatic Polarimeter (Shanghai Shen Guang Instrument Co., Ltd.

### Example 1: Molecular Modeling

### Methodology

### Ligand Preparation

Each ligand used in the study was built using ChemBioOffice, and was energy-minimized using the MMFF94 (23) force-field. Ligand structures were then imported into AMBER (v11) (24) software, AMBER modules were loaded, and antechamber was used to convert the structures to mol2 files with the application of Gasteiger charges. Further missing parameters were then generated using parmchk, which uses the gaff.dat force-field to facilitate this process.

### DNA Preparation

DNA was built in every instance using the nuc module of AMBER. The gaff and DNA optimised parm99bsco (25) force-fields and modified DNA library were loaded for DNA. Parmbsco considers α/γ bond rotations of nucleic acids, reducing fraying of bases over long-scale MD simulations (25).

### Ligand:DNA Adduct Simulation

The AMBER module xleap was used to make initial approximate docking alignments of ligands into the minor groove of DNA (with the appropriate sequence), prior to subsequent energy minimization. The placement was done such that the N₁₀ of the PBD was within 2 Å of the intended exocyclic amino group of the reacting guanine. This was undertaken as the PBD is thought to form a reaction-mediating H-bond with the DNA, which in turn pulls the molecule into the minor groove, and non-covalent simulations allowed the investigation of initial ligand:DNA contacts.

A similar process was undertaken for covalently bound simulations where the ligand was first docked in the DNA minor groove of each individual sequence, and covalent bonds were then created. The CBI was first covalently bound to N3 of the appropriate adenine (parameters for covalent attachment derived in-house), and simulated mono-covalently bound in order to investigate DNA span of unsymmetrical dimers. A third set of simulations was also undertaken where the covalent bond was created between both the N3 of an adenine and the cyclopropane of the CBI and between the exocyclic amine of guanine and the N10-C11 imine of the PBD using the AMBER module xleap. C11S stereochemistry was maintained in every case at the binding interface of the PBD. Parameters for the covalent attachment of the PBD to DNA were created using parameters derived previously through molecular mechanics calculations (26).

Each adduct was then minimized in a stepwise manner to facilitate accommodation in the minor groove. In this procedure, positional restraints are initially used on the DNA atoms to keep their positions fixed and the ligands are then energy minimized alone, followed by full minimization of the system (without restraints) to ensure ligands are accommodated deep in the minor groove.

Production simulations were undertaken in implicit solvent, where the Generalised Born solvation method was used, which is equivalent to the Poisson Boltzmann method, but includes a surface area term to enable accuracy in the simulation of macromolecules (27). A term to allow for monovalent electrostatic ion screening was also employed to simulate the effect of Na⁺ ions in the surrounding environment.

The choice of simulation time is important, and is generally judged upon availability of hardware and time required for the simulation to equilibrate to a degree where potential energy can be considered stable over time. A valuable indicator of this is obtained through plotting conformational variability of the ligand:DNA adduct over time. This is achieved by comparing the coordinates of each frame with the first frame, finding the best RMS fit in each case. In the case of DNA macromolecule simulation (and particularly PBD:DNA adduct simulation), a simulation time of ions in duration in implicit solvent is sufficient to represent the ligand:DNA complex, as simulations of this type are well established in literature (28, 29, 30) using the protocol outlined.

Example RMSD graphs of simulations contained within this study are provided in Supporting Information, proving simulations went to equilibrium as expected. RMSD calculations were also conducted between the lowest energy snapshot of the MD simulations (derived through a ptraj script) and the ligand:DNA adduct structure post full minimization of the system, which provided a numeric measurement of DNA disorder.

### DISCUSSION

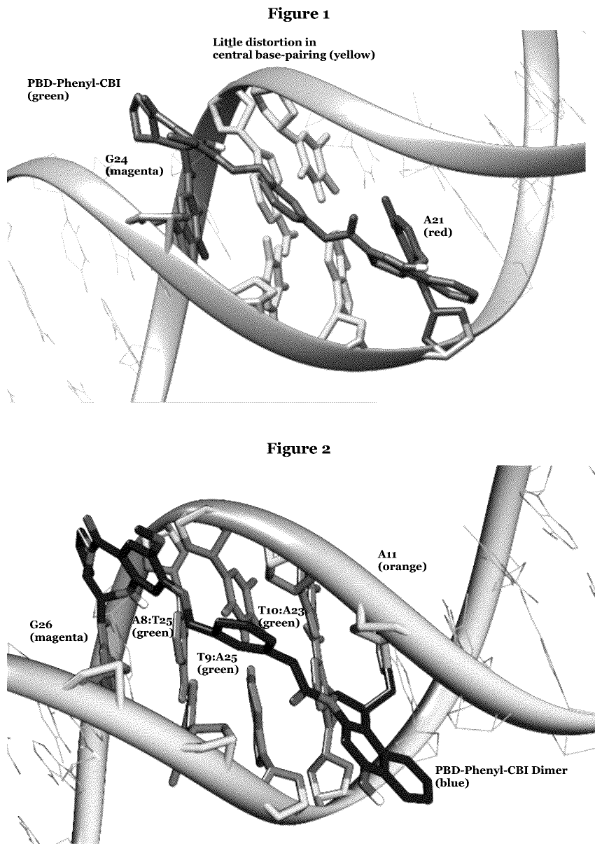

The novel hybrid molecules described herein have been designed through molecular modeling to ensure that they (a) fit snugly in the DNA minor groove, and (b) the two alkylating moieties are in the appropriate positions to cross-link GC and AT base pairs (FIGS. 1 & 2).

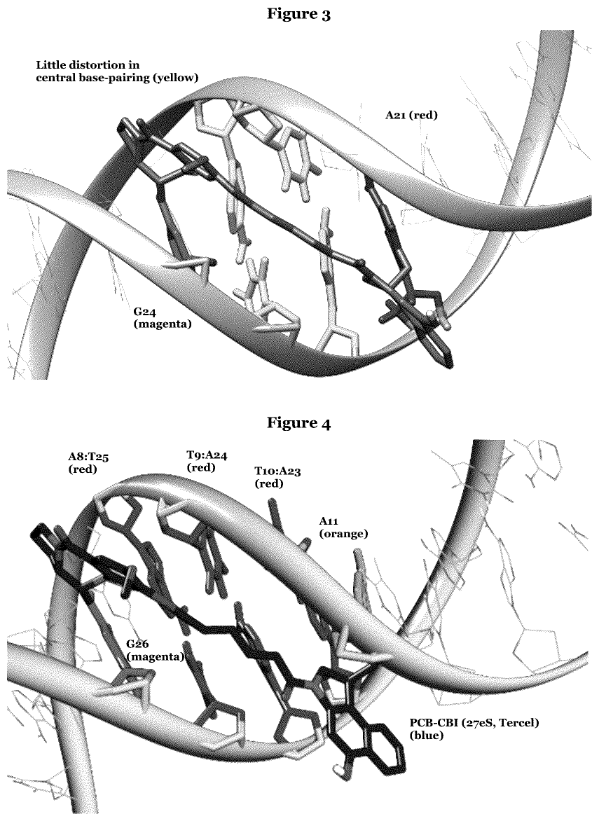

In the case of the PBD-Phenyl-CBI molecule (FIGS. 1 & 2), the central phenyl linker forms extensive van der Waals interactions with the minor groove floor, which in turn stabilizes the adduct formed. For example, the non-covalent binding ability of the dimer is reflected in free energy of binding calculations (kcal/mol) undertaken between the PBD-Phenyl-CBI ligand and DNA sequence 5′-GTATAACATTATATAC-3′, where free energy of binding results suggest strong affinity (−41.55 kcal/mol) with the minor groove. This compares favourably to the known PBD-CBI dimer 27eS (FIGS. 3 & 4) which has free energy of binding of −40.73 kcal/mol with the same sequence, suggesting the phenyl-containing molecule should stabilize DNA to a greater extent than the 27eS molecule.

Furthermore, RMSD calculations were also conducted between the lowest energy snapshot of the MD simulations (derived through a ptraj script) and the ligand:DNA adduct structure post full minimization of the system, which provided a numeric measurement of DNA disorder. In the case of the known PBD-CBI hybrid 27eS, DNA disorder was calculated to be 1.18, whereas slightly less disorder (0.80) was observed in simulations of the PBD-Phenyl-CBI hybrid, again suggesting similar potency as degree of DNA distortion is correlated with DNA-binding ability. Base-pairing was maintained throughout simulation, suggesting stable adducts were generated.

Examples of PDD-CBI molecules connected via the same linkage as the PBD-CBI molecule 27eS also suggest strong affinity with the DNA minor groove, inferring similar potency. For example, the PDD-CBI molecule containing a pentamethylene linker possesses a free energy of binding value of −40.52 kcal/mol and an RMSD value of 1.02, which suggests strong DNA-binding ability and little distortion of DNA.

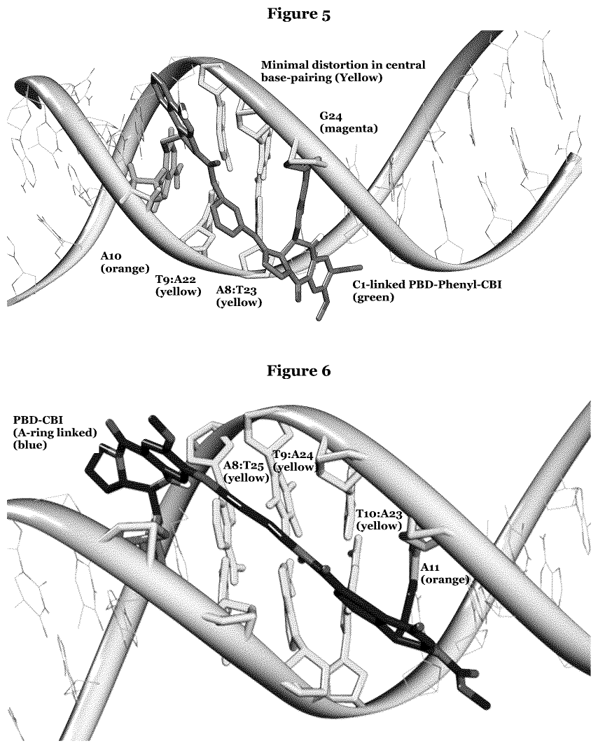

Simulations of the C1-linked PBD-CBI dimer containing a phenyl linker (suggest similar interaction with the minor groove (FIG. 5). The shape fit of the molecule is conducive to DNA minor groove interactivity (as evidenced in non-covalent simulations), and covalently bound simulations illustrate the molecule snugly bound in the minor groove, with the phenyl linker forming extensive non-covalent interactions with the minor groove floor. Furthermore, little distortion of the minor groove was evident in simulations (RMSD of 1.06), and base pairing was maintained, suggesting strong interactivity with the minor groove floor.

The PBD-CBI conjugate linked via C7 on the A-ring of the CBI also exhibits extensive interactions with the minor groove floor. The molecule fits snugly in the minor groove (in a similar manner to the PBD dimers), causing little DNA distortion. The imine of the PBD is ideally located to alkylate DNA (in this instance G26 on the reverse strand), and the CBI is also ideally situated to alkylate an adenine base four base pairs away (i.e., A11). As such, the PBD-CBI conjugate spans five base-pairs (5′-C(G)ATTA-3′), and in the example snapshot (FIG. 6) can be observed to form an interstrand cross-link in DNA. The free energy of binding calculations are similarly favourable, and suggest strong affinity for the minor groove.

Surprisingly, dimers linked via the C8 of the CBI and C8 of the PDD/PBD also suggest comparable binding affinity and accommodation in the minor groove of DNA to those linked via C7.

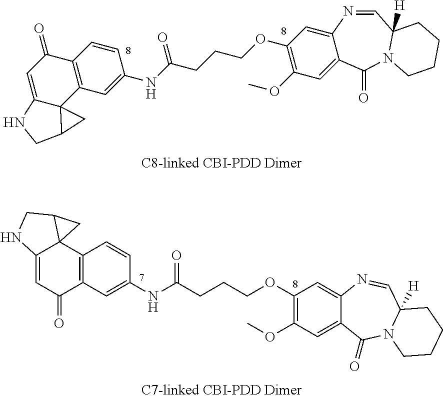 [SMILES: COc1cc2c(cc1OCCCC(=O)Nc1ccc3c(c1)C14CC1CNC4=CC3=O)N=C[C@@H]1CCCCN1C2=O ; COc1cc2c(cc1OCCCC(=O)Nc1ccc3c(c1)C(=O)C=C1NCC4CC134)N=C[C@H]1CCCCN1C2=O]

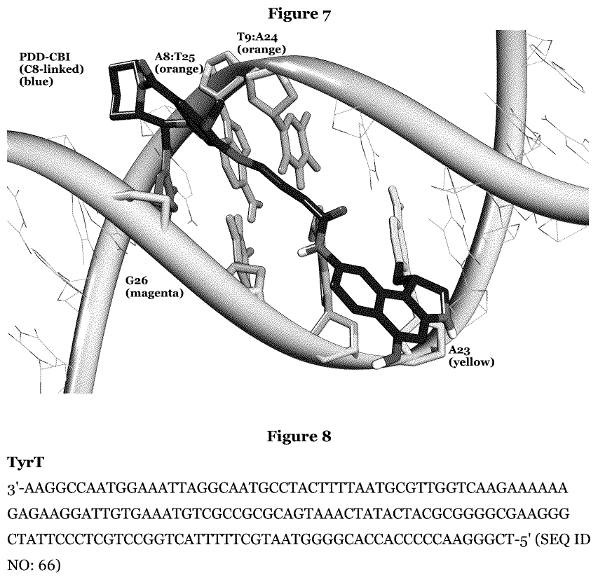

Free energy of binding calculations suggest an affinity of −37.72 kcal/mol for the DNA sequence, which is slightly less favourable than other molecules simulated (Table 1). However, both PBD/PDD and CBI are located at precise orientations in non-covalent simulations, suggesting that alkylation of the guanine (by PBD/PDD) and adenine (by the CBI) would readily occur. The example illustrated below (FIG. 7) shows an intra-strand cross-link, but simulations suggest an inter-strand cross-link is equally likely. Similarly, the central methylene linker of the dimer forms van der Waals interactions with the minor groove floor (particularly thymine residues, in this instance T25 and T9), which assist in stabilizing the adduct. Little DNA distortion occurs, and this is reflected in RMSD calculations (0.78).

TABLE 1

| | | | RMSD | |
|---|---|---|---|---|
| | | | Calculations | |
| | | Free Energy | (generated from) | Base-pairing |
| | DNA Sequence (Span of | of Binding | covalently | maintained |
| Asymmetric | moleucle in non-covalent | Calculations | bound | during |
| molecule | simulations in bold) | (kcal/mol) | simulations) | simulation |
| PBD-CBI (27eS) | 5′-GCTATAACATTATATAC-3′ | −40.73 | 1.18 | Y |
| PBD/PDD- | 5′-GCTATAACATTATATAC-3′ | −41.55 | 0.80 | Y |
| Phenyl-CBI | | | | |
| PDD-CBI | 5′-GCTATAACATTATATAC-3′ | −40.52 | 1.02 | Y |
| C₁PBD-Phenyl- | 5′-GCTATAACATTATATAC-3′ | −43.22 | 1.06 | Y |
| CBI | | | | |
| PBD/PDD-CBI | 5′-GCTATAACATTATATAC-3′ | −41.56 | 0.64 | Y |
| (C₇-linked) | | | | |
| PBD/PDD-CBI | 5′-GCTATAACATTATATAC-3′ | −37.72 | 0.78 | Y |
| (C8-linked) | | | | |

Free energy of binding, RMSD calculations and degree of base-pair maintenance of PBD-CBI and
PDD-CBI molecules in cross-linked DNA sequences (span of molecule highlighted in red).

General Synthetic Scheme for PDD Precursor

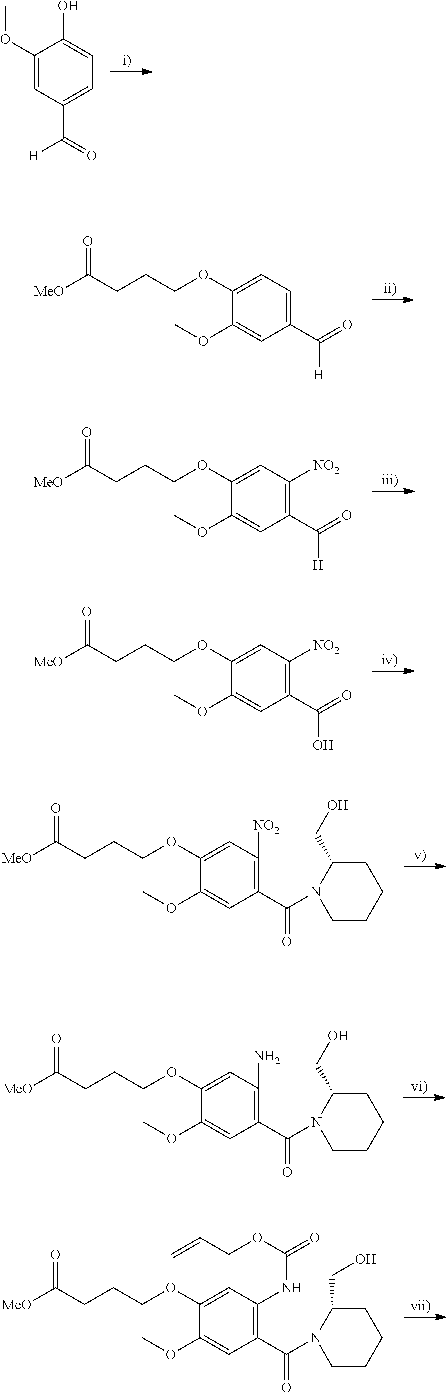

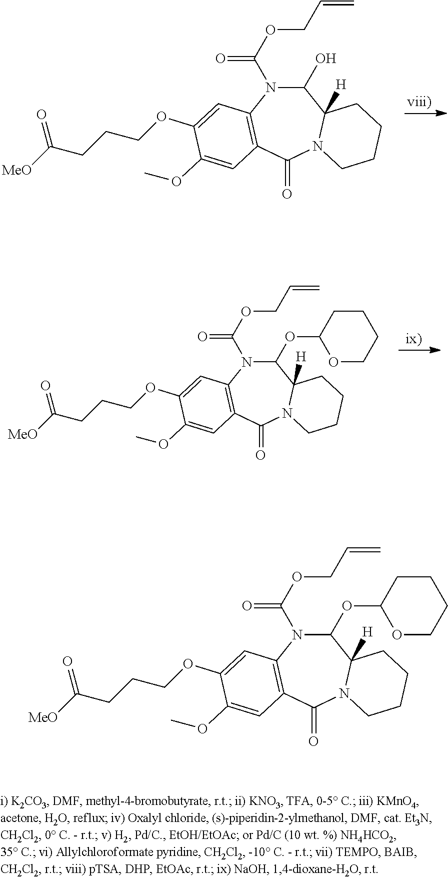 [SMILES: C=CCOC(=O)N1c2cc(OCCCC(=O)OC)c(OC)cc2C(=O)N2CCCC[C@H]2C1O ; C=CCOC(=O)N1c2cc(OCCCC(=O)OC)c(OC)cc2C(=O)N2CCCC[C@H]2C1OC1CCCCO1 ; C=CCOC(=O)N1c2cc(OCCCC(=O)OC)c(OC)cc2C(=O)N2CCCC[C@H]2C1OC1CCCCO1]

### Example 2: Methyl 4-(4-formyl-2-methoxyphenoxy)butanoate (1)

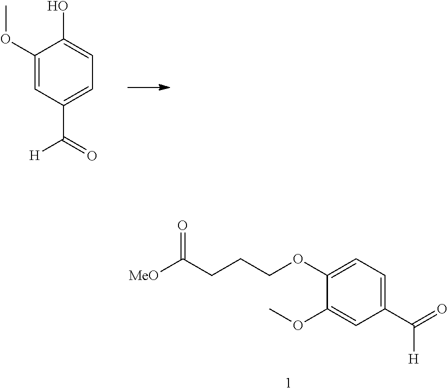 [SMILES: COc1cc(C=O)ccc1O ; COC(=O)CCCOc1ccc(C=O)cc1OC]

A mixture of vanillin (20.0 g, 131 mmol), methyl 4-bromobutanoate (17.5 mL, 139 mmol) and potassium carbonate (27.2 g, 197 mmol) in N,N-dimethylformamide (100 mL) was stirred at room temperature for 18 h. The reaction mixture was diluted with water (500 mL) and the title compound (30.2 g, 91%) was obtained by filtration as a white solid. The product was carried through to the next step without any further purification.

¹H NMR (400 MHz, CDCl₃) δ 9.84 (s, 1H), 7.46-7.37 (m, 2H), 6.98 (d, J=8.2 Hz, 1H), 16 (t, J=6.3 Hz, 2H), 3.91 (s, 3H), 3.69 (s, 3H), 2.56 (t, J=70.2 Hz, 2H), 2.20 (quin, J=6.7 Hz, 2H); ¹³C NMR (100 MHz, CDCl₃) δ 190.9, 173.4, 153.8, 149.9, 130.1, 126.8, 111.6, 109.2, 67.8, 56.0, 51.7, 30.3, 24.2; MS M/Z (EIMS)=271.9 (M+Na)+, 253 (M+H)⁺; LCMS (Method A): t_{R}=6.48 min.

### Example 3: Methyl 4-(4-formyl-2-methoxy-5-nitrophenoxy)butanoate (2)

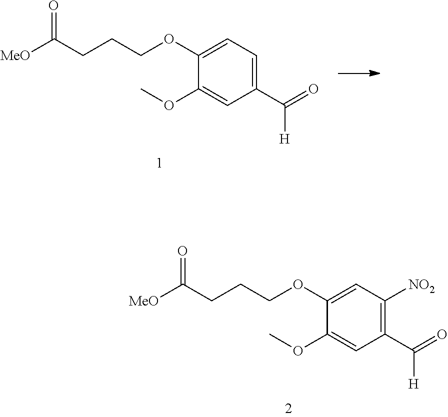 [SMILES: COC(=O)CCCOc1ccc(C=O)cc1OC ; COC(=O)CCCOc1cc([N+](=O)[O-])c(C=O)cc1OC]

To a stirring solution of potassium nitrate (10.0 g, 98.9 mmol) in TFA (50 mL) at 0° C. was added dropwise a solution of methyl 4-(4-formyl-2-methoxyphenoxy)butanoate (1) (20.0 g, 79.2 mmol) in TFA (50 mL). The reaction mixture was stirred at room temperature for 1 h. It was then concentrated in vacuo and diluted with ethyl acetate (400 mL). The organic layer was sequentially washed with brine (3×100 mL) and a saturated aqueous solution of sodium hydrogen carbonate (2×80 mL), dried over sodium sulfate, filtered and concentrated to give the title compound (23.5 g, 100%) as a yellow solid. The product was carried through to the next step without any further purification.

¹H NMR (400 MHz, CDCl₃) δ 10.42 (s, 1H), 7.60 (s, 1H), 7.39 (s, 1H), 4.21 (t, J=6.3 Hz, 2H), 3.98 (s, 3H), 3.70 (s, 3H), 2.61-2.53 (m, 2H), 2.22 (quin, J=6.6 Hz, 2H); ¹³C NMR (100 MHz, CDCl₃) δ 187.8, 173.2, 153.5, 151.7, 143.8, 125.5, 109.9, 108.1, 68.6, 56.6, 51.8, 30.2, 24.1; MS M/Z (EIMS)=298 (M+H), 296.1 (M−H)⁻; LCMS (Method A): t_{R}=6.97 min.

### Example 4: 5-Methoxy-4-(4-methoxy-4-oxobutoxy)-2-nitrobenzoic acid (3)

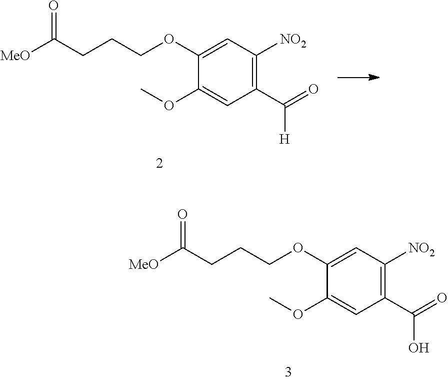 [SMILES: COC(=O)CCCOc1cc([N+](=O)[O-])c(C=O)cc1OC ; COC(=O)CCCOc1cc([N+](=O)[O-])c(C(=O)O)cc1OC]

To a solution of methyl 4-(4-formyl-2-methoxy-5-nitrophenoxy)butanoate (2) (23.0 g, 77.4 mmol) in acetone (600 mL) was quickly added a hot (70° C.) solution of potassium permanganate (46.0 g, 291 mmol) in water (400 mL). The reaction mixture was stirred at 70° C. for 3 h. The reaction mixture was cooled to room temperature and passed through celite. The cake of celite was washed with hot water (200 mL). A solution of sodium bisulfite in HCl (1 M, 200 mL) was added to the filtrate which was extracted with dichloromethane (2×400 mL). The combined organic extracts were then was dried over sodium sulfate, filtered and concentrated. The resulting residue was purified by column chromatography (silica), eluting with methanol/dichloromethane (from 0% to 50%), to give the title compound (17.0 g, 70%) as a pale yellow solid.

¹H NMR (400 MHz, MeOD) δ 7.47 (s, 1H), 7.25 (s, 1H), 4.13 (t, J=6.2 Hz, 2H), 3.94 (s, 3H), 3.68 (s, 3H), 2.54 (t, J=70.2 Hz, 2H), 2.17-2.06 (m, 2H); ¹³C NMR (100 MHz, MeOD) δ 175.3, 168.6, 153.8, 151.3, 143.1, 122.8, 112.4, 109.2, 69.6, 57.0, 52.2, 31.2, 25.5; MS M/Z (EIMS)=314 (M+H)⁺, 311.9 (M−H)⁻; LCMS (Method A): t_{R}=6.22 min.

### Example 5: Methyl (S)-4-(4-(2-(hydroxymethyl)piperidine-1-carbonyl)-2-methoxy-5-nitrophenoxy)butanoate (4)

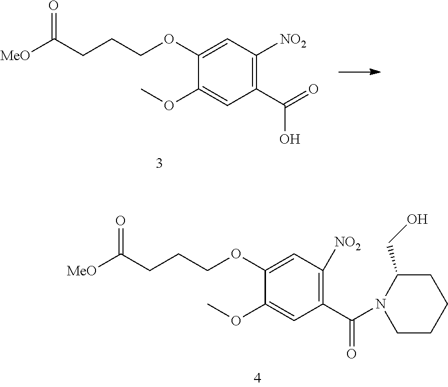 [SMILES: COC(=O)CCCOc1cc([N+](=O)[O-])c(C(=O)O)cc1OC ; COC(=O)CCCOc1cc([N+](=O)[O-])c(C(=O)N2CCCC[C@H]2CO)cc1OC]

A mixture of 5-methoxy-4-(4-methoxy-4-oxobutoxy)-2-nitrobenzoic acid (3) (8.0 g, 25.5 mmol), oxalyl chloride (6.6 mL, 77.0 mmol) and anhydrous N,N-dimethl-formamide (2 drops) in anhydrous dichloromethane (100 mL) was stirred at room temperature for 1 h. Anhydrous toluene (20 mL) was added to the reaction mixture which was then concentrated in vacuo. A solution of the resulting residue in anhydrous dichloromethane (10 mL) was added dropwise to a solution of (S)-piperidin-2-ylmethanol (3.8 g, 33.4 mmol) and triethylamine (10.7 mL, 77.0 mmol) in anhydrous dichloromethane (90 mL) at −10° C. The reaction mixture was stirred at room temperature for 2 h and then washed with hydrochloric acid (1 M, 50 mL) and brine (50 mL), dried over sodium sulfate, filtered and concentrated. The resulting residue was purified by column chromatography (silica), eluting with methanol/dichloro-methane (from 0% to 5%), to give the title compound (9.2 g, 73%) as a yellow oil.

¹H NMR (400 MHz, CDCl₃) δ 7.68-7.64 (m, 1H), 6.77-6.70 (m, 1H), 4.16-4.07 (m, 3H), 3.93-3.89 (m, 3H), 3.83 (s, 1H), 3.67 (s, 3H), 3.15 (d, J=1.4 Hz, 1H), 3.11 (s, 1H), 2.78 (s, 1H), 2.56-2.50 (m, 3H), 2.21-2.12 (m, 4H), 1.74-1.55 (m, 4H); ¹³C NMR (100 MHz, CDCl₃) δ 173.3, 168.1, 154.6, 148.2, 137.4, 127.6, 111.4, 108.3, 68.3, 60.6, 56.7, 53.5, 51.7, 43.3, 38.0, 34.9, 30.3, 24.1, 19.7; MS M/Z (EIMS)=411.0 (M+H)⁺; LCMS (Method A): t_{R}=6.28 min.

### Example 6: Methyl (S)-4-(5-amino-4-(2-(hydroxymethyl)piperidine-1-carbonyl)-2-methoxyphenoxy)butanoate (5)

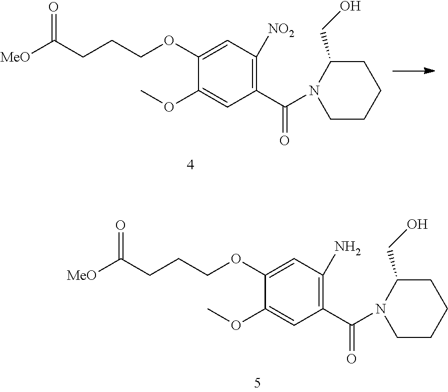 [SMILES: COC(=O)CCCOc1cc([N+](=O)[O-])c(C(=O)N2CCCC[C@H]2CO)cc1OC ; COC(=O)CCCOc1cc(N)c(C(=O)N2CCCC[C@H]2CO)cc1OC]

To a solution of methyl (S)-4-(4-(2-(hydroxymethyl)piperidine-1-carbonyl)-2-methoxy-5-nitrophenoxy)butanoate (4) (9.2 g, 22.4 mmol) in ethanol (40 mL) and ethyl acetate (10 mL) was added palladium on activated charcoal (10% wt. basis) (920 mg). The reaction mixture was hydrogenated at 35 psi for 3 h in a Parr apparatus. The reaction mixture was filtered through celite and the resulting cake was washed with ethyl acetate. The filtrate was concentrated in vacuo to give the title compound (9.0 g, 90%) as a pink solid. The product was carried through to the next step without any further purification.

¹H NMR (400 MHz, CDCl₃) δ 6.69 (s, 1H), 6.27-6.18 (m, 1H), 4.03-3.94 (m, 3H), 3.94-3.82 (m, 3H), 3.81-3.76 (m, 1H), 3.74 (s, 3H), 3.73-3.68 (m, 1H), 3.67-3.65 (m, 3H), 3.56 (d, J=40.8 Hz, 1H), 3.03 (s, 1H), 2.51 (t, J=70.2 Hz, 2H), 2.11 (quin, J=6.7 Hz, 2H), 1.68-1.59 (m, 4H), 1.55-1.40 (m, 2H); ¹³C NMR (100 MHz, CDCl₃) δ 173.6, 171.2, 150.3, 141.8, 141.1, 113.2, 112.3, 102.4, 67.5, 60.8, 60.4, 56.8, 51.6, 30.4, 25.8, 24.3, 21.0, 19.9, 14.2; MS M/Z (EIMS)=381.0 (M+H)⁺; LCMS (Method A): t_{R}=5.52 min.

### Example 7: Methyl (S)-4-(5-(((allyloxy)carbonyl)amino)-4-(2-(hydroxyl-methyl)piperidine-1-carbonyl)-2-methoxyphenoxy)butanoate (6)

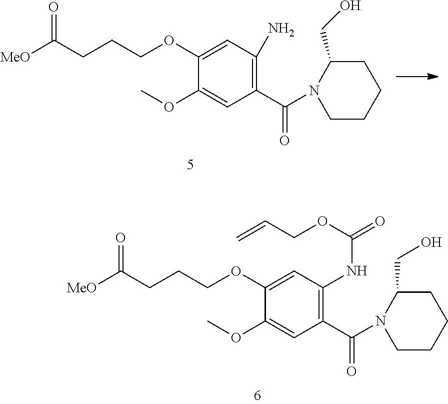 [SMILES: COC(=O)CCCOc1cc(N)c(C(=O)N2CCCC[C@H]2CO)cc1OC ; C=CCOC(=O)Nc1cc(OCCCC(=O)OC)c(OC)cc1C(=O)N1CCCC[C@H]1CO]

To a solution of methyl (S)-4-(5-amino-4-(2-(hydroxymethyl)piperidine-1-carbonyl)-2-methoxyphenoxy)butanoate (5) (9.0 g, 23.7 mmol) and pyridine (4.4 mL, 54.4 mmol) in anhydrous dichloromethane (100 mL) at −10° C. was added dropwise a solution of allylchloroformate (2.6 mL, 24.8 mmol) in anhydrous dichloromethane (20 mL). The reaction mixture was stirred at room temperature for 30 min. The reaction mixture was sequentially washed with a saturated aqueous solution of copper (II) sulfate (80 mL), water (80 mL) and a saturated aqueous solution of sodium hydrogen carbonate (80 mL). The organic layer was dried over sodium sulfate, filtered and concentrated. The resulting residue (2.0 g out of the 11.0 g crude) was purified by column chromatography (silica), eluting with methanol/dichloromethane (from 0% to 1%), to give the title compound (930 mg, 47% based on the amount purified) as a yellow oil.

¹H NMR (400 MHz, CDCl₃) δ 8.30 (br s, 1H), 7.63 (br s, 1H), 6.76 (br s, 1H), 5.92 (ddt, J=17.2, 10.6, 5.4, 5.4 Hz, 1H), 5.37-5.28 (m, 1H), 5.20 (dq, J=10.4, 1.3 Hz, 1H), 4.65-4.56 (m, 2H), 4.06 (t, J=6.2 Hz, 2H), 3.94-3.82 (m, 1H), 3.79 (s, 3H), 3.66 (s, 3H), 3.62-3.54 (m, 1H), 3.40 (br s, 1H), 3.10-2.88 (m, 1H), 2.52 (t, J=7.4 Hz, 2H), 2.22-2.04 (m, 3H), 1.64 (br s, 4H), 1.56-1.31 (m, 2H); ¹³C NMR (100 MHz, CDCl₃) δ 173.5, 170.6, 153.9, 149.7, 144.8, 132.6, 130.1, 117.6, 116.9, 110.8, 107.1, 106.0, 67.7, 65.6, 60.7, 56.3, 53.5, 51.6, 43.1, 30.5, 25.7, 24.4, 19.7; MS M/Z (EIMS)=465.1 (M+H)⁺; LCMS (Method A: t_{R}=6.47 min.

### Example 8: Allyl (6S,6aS)-6-hydroxy-2-methoxy-3-(4-methoxy-4-oxobutoxy)-12-oxo-6,6a,7,8,9,10-hexahydrobenzo[e]pyrido[1,2-a][1,4]diazepine-5(12H)-carboxylate (71

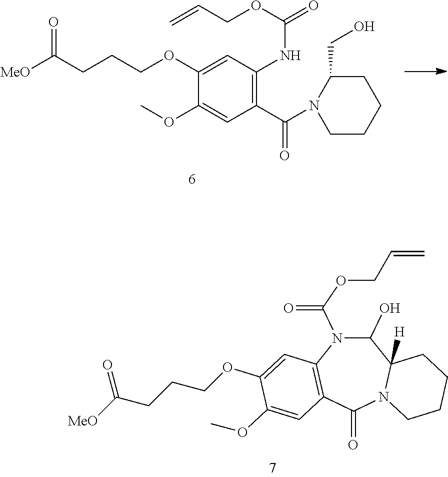 [SMILES: C=CCOC(=O)Nc1cc(OCCCC(=O)OC)c(OC)cc1C(=O)N1CCCC[C@H]1CO ; C=CCOC(=O)N1c2cc(OCCCC(=O)OC)c(OC)cc2C(=O)N2CCCC[C@H]2C1O]

To a solution of methyl (S)-4-(5-(((allyloxy)carbonyl)amino)-4-(2-(hydroxymethyl)-piperidine-1-carbonyl)-2-methoxyphenoxy)butanoate (6) (930 mg, 2.0 mmol) in dichloromethane (45 mL) was added 2,2,6,6-tetramethyl-piperidin-1-yl)oxyl (TEMPO) (32 mg, 0.20 mmol) and (diacetoxyiodo)-benzene (773 mg, 2.4 mmol). The reaction mixture was stirred at room temperature for 16 h, and was then sequentially washed with a saturated aqueous solution of sodium metabisulfite (20 mL), a saturated aqueous solution of sodium hydrogen carbonate (20 mL), water (20 mL) and brine (20 mL). The organic layer was then dried over sodium sulfate, filtered and concentrated. The resulting residue was purified by column chromatography (silica), eluting with methanol/dichloromethane (from 0% to 5%), to give the title compound (825 mg, 89%) as a cream solid.

MS M/Z (EIMS)=462.9 (M+H)⁺; LCMS (Method A): t_{R}=6.30 min.

### Example 9: Allyl (6S,6aS)-2-methoxy-3-(4-methoxy-4-oxobutoxy)-12-oxo-6-((tetrahydro-2H-pyran-2-yl)oxy)-6,6a,7,8,9,10-hexahydrobenzo[e]pyrido-[1,2-a][1,4]diazepine-5(12H)-carboxylate (8)

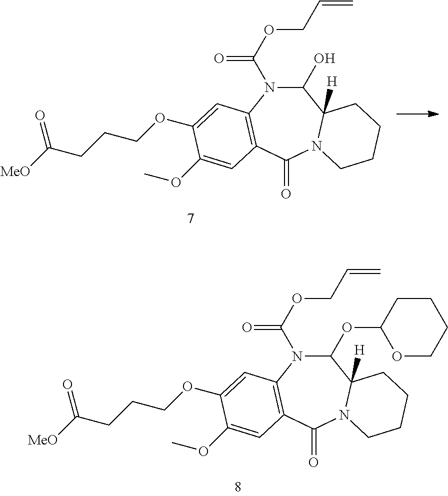 [SMILES: C=CCOC(=O)N1c2cc(OCCCC(=O)OC)c(OC)cc2C(=O)N2CCCC[C@H]2C1O ; C=CCOC(=O)N1c2cc(OCCCC(=O)OC)c(OC)cc2C(=O)N2CCCC[C@H]2C1OC1CCCCO1]

A mixture of allyl (6S,6aS)-6-hydroxy-2-methoxy-3-(4-methoxy-4-oxobutoxy)-12-oxo-6,6a,7,8,9,10-hexahydrobenzo[e]pyrido[1,2-a][1,4]diazepine-5(12H)-carboxylate (7) (825 mg, 1.8 mmol), 3,4-dihydro-2H-pyran (1.7 mL, 18.2 mmol) andp-toluenesulfonic acid monohydrate (pTSA) (8.5 mg, 1% w/w) in ethyl acetate (12 mL) was stirred at room temperature for 16 h. The reaction mixture was then diluted with ethyl acetate (50 mL) and washed with a saturated aqueous solution of sodium hydrogen carbonate (20 mL) and brine (30 mL). The organic layer was dried over sodium sulfate, filtered and concentrated. The resulting residue was purified by column chromatography (silica), eluting with methanol/dichloromethane (from 0% to 2%), to give the title compound (820 mg, 84%) as a cream solid.

MS M/Z (EIMS)=546.7 (M+H)⁺; LCMS (Method A): t_{R}=7.70 min.

### Example 10: 4-(((6S,6aS)-5-((Allyloxy)carbonyl)-2-methoxy-12-oxo-6-((tetra-hydro-2H-pyran-2-yl)oxy)-5,6,6a,7,8,9,10,12-octahydrobenzo[e]pyrido[1,2-a][1,4]diazepin-3-yl)oxy)butanoic acid (9)

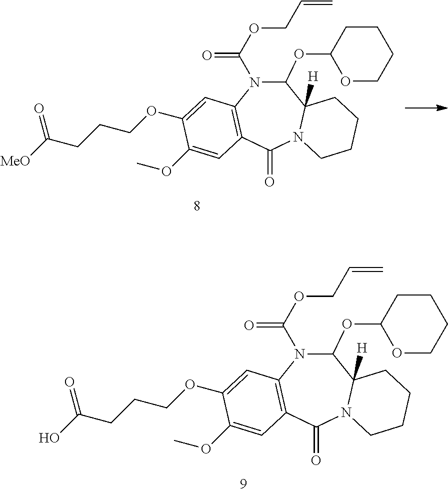 [SMILES: C=CCOC(=O)N1c2cc(OCCCC(=O)OC)c(OC)cc2C(=O)N2CCCC[C@H]2C1OC1CCCCO1 ; C=CCOC(=O)N1c2cc(OCCCC(=O)O)c(OC)cc2C(=O)N2CCCC[C@H]2C1OC1CCCCO1]

To a solution of allyl (6S,6aS)-2-methoxy-3-(4-methoxy-4-oxobutoxy)-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (8) (770 mg, 1.4 mmol) in 1,4-dioxane (10 mL) was added a 0.5 M aqueous solution of sodium hydroxide (10 mL, 5.0 mmol). The reaction mixture was stirred at room temperature for 2 h and was then concentrated in vacuo, after which water (20 mL) was added and the aqueous layer was acidified to pH=1 with an aqueous 1 M citric acid solution (5 mL). The aqueous layer was then extracted with ethyl acetate (2×50 mL). The combined organic extracts were then washed with brine (50 mL), dried over sodium sulfate, filtered and concentrated to give the title compound (700 mg, 93%) as a yellow oil. The product was carried through to the next step without any further purification.

MS M/Z (EIMS)=532.9 (M+H)⁺; LCMS (Method A): t_{R}=6.98 min.

Reaction Scheme for Preparing Compound (11)

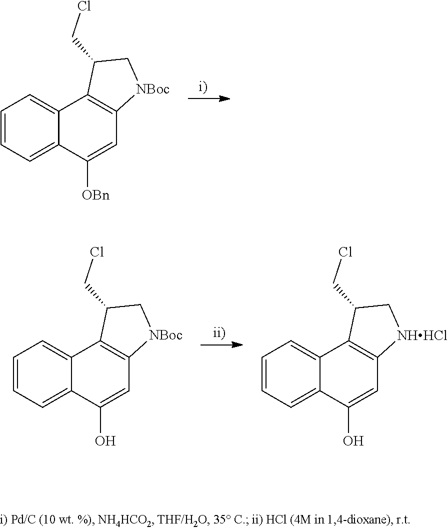

### Example 11: tert-Butyl (S)-1-(chloromethyl)-5-hydroxy-1,2-dihydro-3H-benzo[e]indole-3-carboxylate (10)

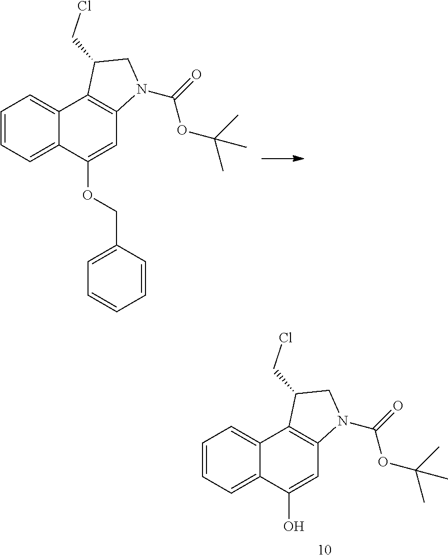 [SMILES: CC(C)(C)OC(=O)N1C[C@@H](CCl)c2c1cc(OCc1ccccc1)c1ccccc21 ; CC(C)(C)OC(=O)N1C[C@@H](CCl)c2c1cc(O)c1ccccc21]

A solution of tert-butyl (S)-5-(benzyloxy)-1-(chloromethyl)-1,2-dihydro-3H-benzo[e]-indole-3-carboxylate (460 mg, 1.09 mmol) in tetrahydrofuran (10 mL) was charged with palladium on activated charcoal (10 wt. % basis) (230 mg) and a solution of ammonium formate (547 mg, 8.68 mmol) in water (2 mL) and then heated to 35° C. under an inert atmosphere of argon. After 1 h, the mixture was allowed to cool and filtered through a pad of celite, which was then washed with ethyl acetate. After extracting the filtrate with ethyl acetate (2×50 mL), the combined organic extracts were dried over magnesium sulfate and concentrated in vacuo. The residue was then purified by recrystallisation (ethyl acetate/hexane) to give the title compound (244 mg, 67%) as a white solid.

¹H NMR (400 MHz, CDCl₃) S8.23 (d, J=8.3 Hz, 1H), 7.86 (br s, 1H), 7.63 (d, J=8.3, 1H), 7.50 (ddt, J=6.8, 1.4, 1.3 Hz, 1H), 7.38-7.33 (m, 1H), 4.27 (d, J=11.5 Hz, 1H), 4.17-4.09 (m, 1H), 3.95 (tt, J=10.0, 2.8 Hz, 2H), 3.43 (t, J=11.5 Hz, 1H), 1.64 (s, 9H); ¹³C NMR (100 MHz, CDCl₃) δ 154.1, 153.2, 141.1, 130.4, 127.6, 123.7, 122.9, 121.8, 121.7, 114.3, 99.2, 81.9, 53.2, 46.5, 41.8, 28.6; MS M/Z (ES−)=332 (M−1)⁻; LCMS (Method C): t_{R}=3.73 min.

### Example 12: (S)-1-(Chloromethyl)-2,3-dihydro-1H-benzo[e]indol-5-ol hydrochloride (11)

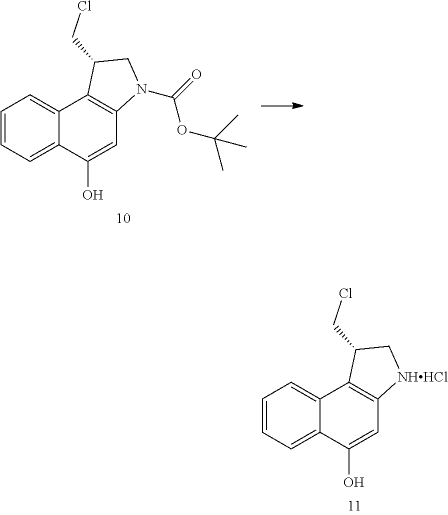

A solution of tert-butyl (S)-1-(chloromethyl)-5-hydroxy-1,2-dihydro-3H-benzo[e]-indole-3-carboxylate (10) (30 mg, 0.090 mmol) in hydrochloric acid (4 M in 1,4-dioxane) was stirred at room temperature under argon. Progress was monitored by LCMS and after approximately 1 h, the reaction mixture was concentrated in vacuo to give the title compound (24 mg, quant.) as a pale green crystalline solid (unstable), which was used immediately in the subsequent step without further purification. MS M/Z (EIMS)=234 (M+H)⁺; LCMS (Method C): t_{R}=2.62 min.

### Example 13: Synthesis of an Asymmetric Conjugate Compound (13)

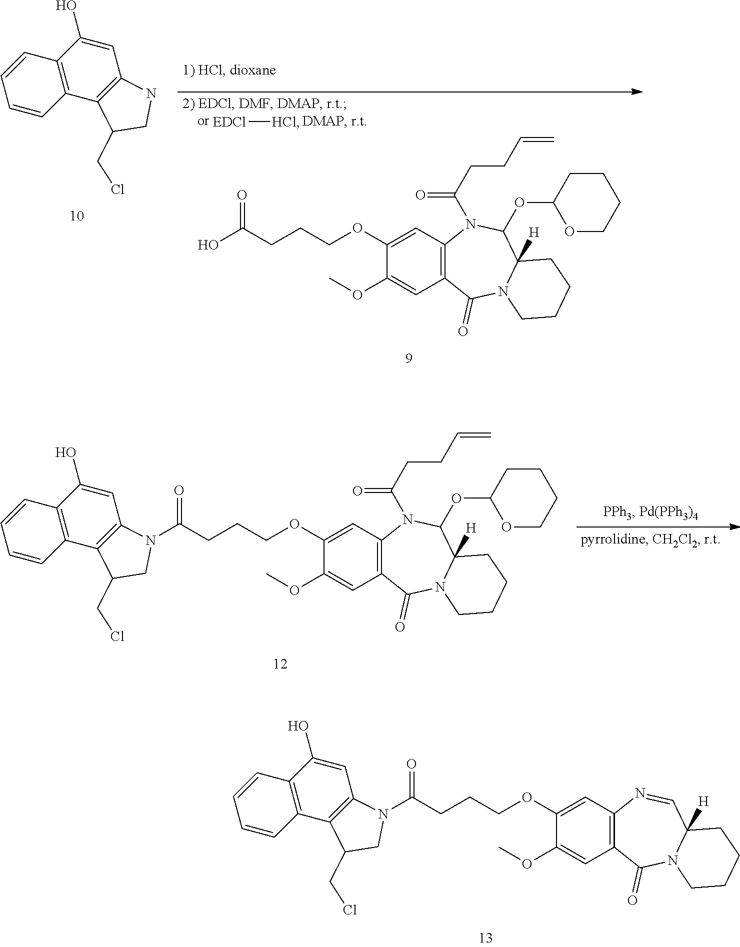

Deprotection of (S)-tert-butyl 1-(chloromethyl)-5-hydroxy-1H-benzo[e]indole-3(2H)-carboxylate (10) [Sigma-Aldrich] is carried out under acid catalysis to provide the crude hydrochloride salt (ii) which is then coupled with the protected PDD compound (9) using 1-(3-dimethylaminopropyl)-3-ethylcarbodiimide hydrochloride and 4-(dimethylamino)pyridine or 1-(3-dimethylaminopropyl)-3-ethylcarbodiimide hydrochloride in N,N-dimethylacetamide to give the protected asymmetric conjugate compound (12). Deprotection of (12) along with imine formation is achieved by reacting (12) with tetrakis(triphenylphosphine)palladium(0) in the presence of triphenyl-phosphine in pyrrolidine and dichloromethane results in (13).

Analogous compounds comprising a PBD unit can be prepared using a protected PBD compound that is equivalent to (9). The synthesis of such protected PBD compounds is disclosed in Wo 2007/039752 and WO 2013/164593.

### Example 14: Allyl (6S,6aS)-3-(4-((S)-1-(chloromethyl)-5-hydroxy-1,2-dihydro-1H-benzo[e]indol-3-yl)-4-oxobutoxy)-2-methoxy-12-oxo-6-((tetrahydro-2H-pyran-2-yl)oxy)-6,6a,7,8,9,10-hexahydrobenzo[e]-pyrido[1,2-a][1,4]diazepine-5(12H)-carboxylate (12)

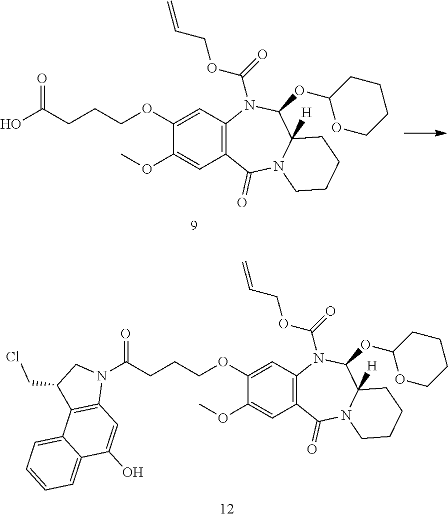 [SMILES: C=CCOC(=O)N1c2cc(OCCCC(=O)O)c(OC)cc2C(=O)N2CCCC[C@H]2[C@@H]1OC1CCCCO1 ; C=CCOC(=O)N1c2cc(OCCCC(=O)N3C[C@@H](CCl)c4c3cc(O)c3ccccc43)c(OC)cc2C(=O)N2CCCC[C@H]2[C@@H]1OC1CCCCO1]

A solution of 4-(((6S,6aS)-5-((allyloxy)carbonyl)-2-methoxy-12-oxo-6-((tetrahydro-2H-pyran-2-yl)oxy)-5,6,6a,7,8,9,10,12-octahydrobenzo[e]pyrido[1,2-a][1,4]diazepin-3-yl)oxy)butanoic acid (9) (97.0 mg, 0.183 mmol) in N,N-dimethylacetamide (2.5 mL) was charged with (S)-1-(chloromethyl)-2,3-dihydro-1H-benzo[e]indol-5-ol hydrochloride (11) (49.0 mg, 0.183 mmol) and N-(3-dimethylaminopropyl)-N′-ethylcarbodiimide hydrochloride (70.0 mg, 0.365 mmol) and stirred at room temperature under argon for 18 h. The reaction mixture was subsequently quenched with a saturated aqueous solution of sodium hydrogen carbonate, then taken up into ethyl acetate, separated and extracted with ethyl acetate (2×50 mL). The combined organic extracts were then washed with brine (50 mL), dried over magnesium sulfate and concentrated in vacuo. Column chromatography (silica), eluting with ethyl acetate/hexane (from 25% to 100%) afforded the title compound (18 mg, 14%) as a pale green oil.

¹H NMR (400 MHz, CDCl₃) δ 8.28 (d, J=8.2 Hz, 1H), 8.11 (br s, 1H), 7.65 (d, J=8.2 Hz, 1H), 7.50 (t, J=70.6 Hz, 1H), 7.36 (t, J=7.3 Hz, 1H), 7.28 (s, 1H), 7.18 (br s, 1H), 6.18 (d, J=9.3 Hz, 1H), 5.78-5.64 (m, 1H), 5.13-4.96 (m, 2H), 4.54 (d, J=11.2 Hz, 1H), 4.39 (br s, 1H), 4.26 (d, J=70.6 Hz, 2H), 4.18 (d, J=9.4 Hz, 1H), 4.02 (d, J=8.3 Hz, 1H), 3.95 (d, J=8.3 Hz, 1H), 3.89 (s, 3H), 3.86 (br s, 1H), 3.82 (d, J=90.1 Hz, 1H), 3.74-3.65 (m, 1H), 3.56-3.44 (m, 2H), 3.39 (t, J=10.7 Hz, 2H), 2.81-2.70 (m, 1H), 2.68-2.62 (m, 1H), 2.39-2.23 (m, 3H), 1.85-1.74 (m, 2H), 1.71-1.63 (m, 4H), 1.54-1.38 (m, 6H); ¹³C NMR (100 MHz, CDCl₃) δ 171.1, 169.4, 155.0, 149.1, 141.4, 138.7, 135.1, 130.0, 129.3, 127.6, 124.0, 123.3, 122.7, 122.6, 121.9, 118.8, 117.3, 114.3, 113.2, 110.5, 100.3, 94.0, 83.8, 66.7, 63.8, 56.1, 55.7, 53.2, 46.4, 45.4, 42.3, 38.8, 38.1, 35.5, 30.4, 25.2, 23.3, 22.9, 21.4, 18.2; MS M/Z (EIMS)=770 (M+Na)⁺; MS M/Z (ES−)=746 (M−1)⁻; LCMS (Method C): t_{R}=3.77 min.

### Example 15: (S)-3-(4-((S)-1-(Chloromethyl)-5-hydroxy-1,2-dihydro-3H-benzo[e]indol-3-yl)-4-oxobutoxy)-2-methoxy-7,8,9,10-tetrahydrobenzo[e]-pyrido[1,2-a][1,4]diazepin-12(6aH)-one (13)

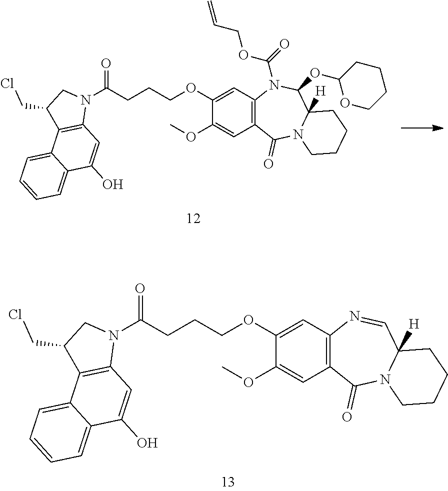 [SMILES: C=CCOC(=O)N1c2cc(OCCCC(=O)N3C[C@@H](CCl)c4c3cc(O)c3ccccc43)c(OC)cc2C(=O)N2CCCC[C@H]2[C@@H]1OC1CCCCO1 ; COc1cc2c(cc1OCCCC(=O)N1C[C@@H](CCl)c3c1cc(O)c1ccccc31)N=C[C@@H]1CCCCN1C2=O]

A solution of allyl (6S,6aS)-3-(4-((S)-1-(chloromethyl)-5-hydroxy-1,2-dihydro-3H-benzo [e]indol-3-yl)-4-oxobutoxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (12) (17.5 mg, 0.023 mmol) in dichloromethane (1 mL) was charged with tetrakis(triphenylphosphine)palladium(0) (1 mg) and pyrrolidine (10 μL) and then stirred at room temperature under argon. After approximately 1 min, the resulting mixture was concentrated in vacuo and immediately purified by column chromateography, eluting with methanol/ethyl acetate (from 0% to 10%), to give the title compound (2.5 mg, 19%) as a yellow oil.

¹H NMR (400 MHz, CDCl₃) δ 9.27 (br s, 1H), 8.28 (d, J=8.4 Hz, 1H), 8.21 (s, 1H), 7.88 (d, J=5.7 Hz, 1H), 7.64 (d, J=8.4 Hz, 1H), 7.51 (t, J=7.7 Hz, 1H), 7.41 (s, 1H), 7.38 (t, J=7.7 Hz, 1H), 6.85 (s, 1H), 4.36-4.19 (m, 4H), 4.06-4.00 (m, 1H), 3.96-3.91 (m, 2H), 3.84 (s, 3H), 3.72 (dd, J=10.0, 4.4 Hz 1H), 3.40 (dd, J=10.8, 4.8 Hz, 1H), 3.23 (ddd, J=14.1, 10.8, 4.0 Hz, 1H), 2.90-2.86 (m, 1H), 2.80-2.76 (m, 1H), 2.47-2.34 (m, 2H), 1.89-1.62 (m, 6H); ¹³C NMR (100 MHz, acetone-d₆) δ 170.6, 166.8, 164.0, 160.3, 150.8, 147.9, 142.4, 140.3, 130.4, 127.2, 123.2, 122.7, 122.4, 121.3, 114.4, 113.5, 111.7, 110.0, 100.4, 67.8, 59.6, 55.4, 52.9, 49.6, 47.0, 41.7, 39.1, 31.7, 24.2, 22.9, 18.2; MS M/Z (EIMS)=562 (M+H)⁺; LCMS (Method C): t_{R}=3.28 min, LCMS (Method A): t_{R}=6.85 min; HRMS (ESI) calculated for: [C₃₁H₃₃ClN₃O₅]⁺: 562.2103, found: 562.2098.

General Reaction Scheme for Asymmetric Conjugate Compound (24)

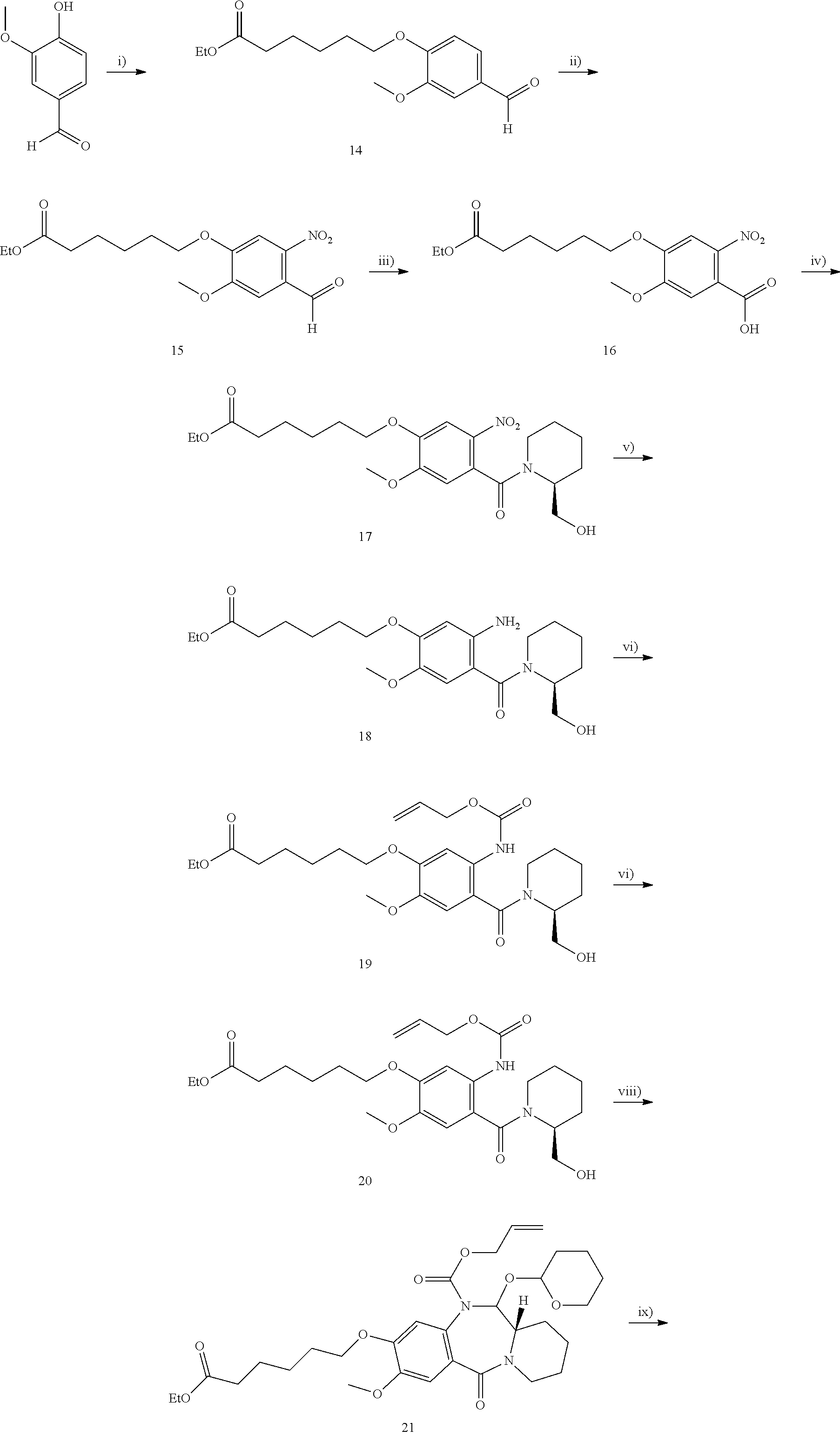

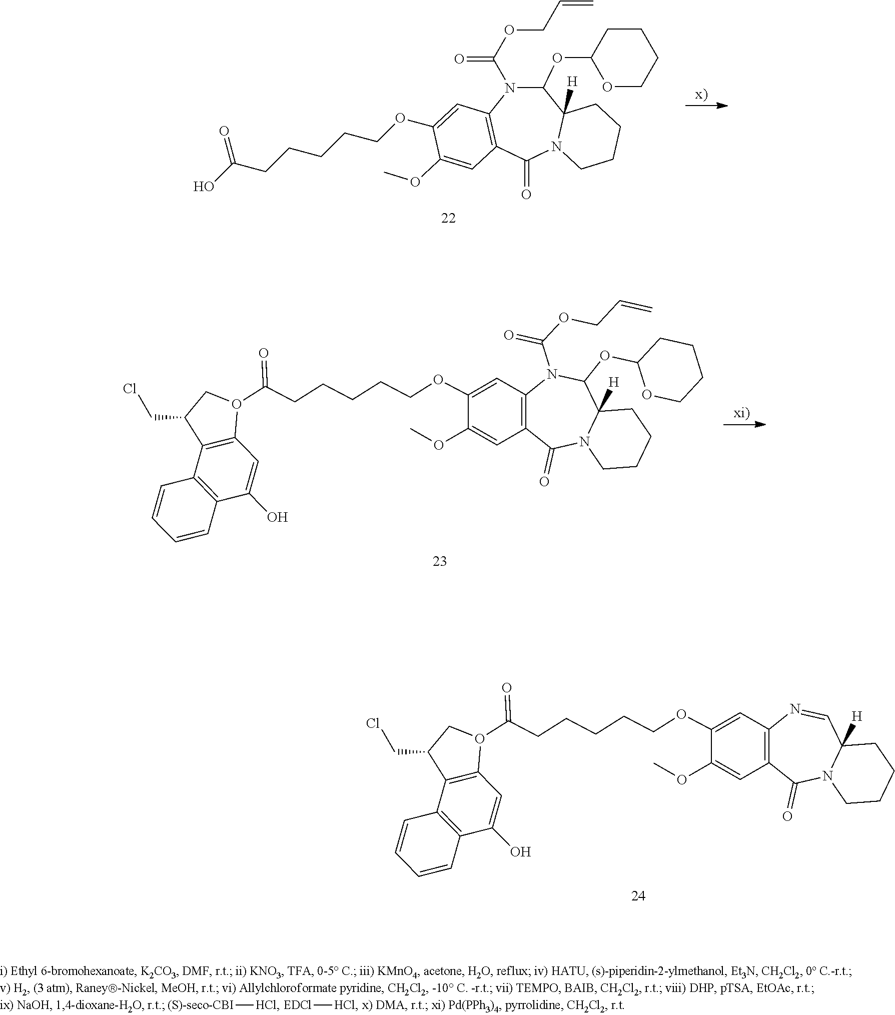

### Example 16: Ethyl 6-(4-formyl-2-methoxyphenoxy)hexanoate (14)

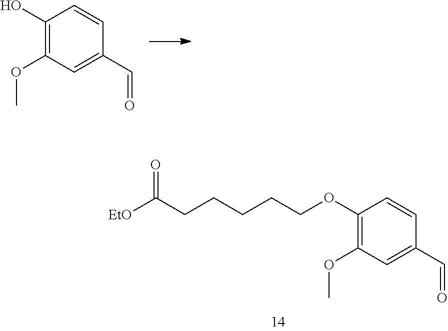 [SMILES: COc1cc(C=O)ccc1O ; CCOC(=O)CCCCCOc1ccc(C=O)cc1OC]

A solution of vanillin (6.50 g, 42.7 mmol), ethyl 6-bromohexanoate (8.00 mL, 45.0 mmol) and potassium carbonate (8.70 g, 63.0 mmol) in N,N-dimethylformamide (50 mL) was stirred at room temperature for 18 h. The reaction mixture was then diluted with water (100 mL), separated and extracted with ethyl acetate (120 mL). The combined organic extracts were sequentially washed with water (100 mL), brine (100 mL), dried over magnesium sulfate, filtered and concentrated to give the title compound (12.5 g, 99%) as a yellow oil, which was carried through to the next step without any further purification.

¹H NMR (400 MHz, CDCl₃) δ 9.84 (s, 1H), 7.43 (dd, J=8.1, 1.9 Hz, 1H), 7.40 (d, J=1.9 Hz, 1H), 6.96 (d, J=8.1 Hz, 1H), 4.08-4.15 (m, 4H), 3.92 (s, 3H), 2.34 (t, J=7.5 Hz, 2H), 1.87-1.94 (m, 2H), 1.68-1.75 (m, 2H), 1.49-1.56 (m, 2H), 1.25 (t, J=70.2 Hz, 3H); MS M/Z (EIMS)=317 (M+Na)⁺; LCMS (Method B): t_{R}=3.82 min.

### Example 17: Ethyl 6-(4-formyl-2-methoxy-5-nitrophenoxy)hexanoate (15)

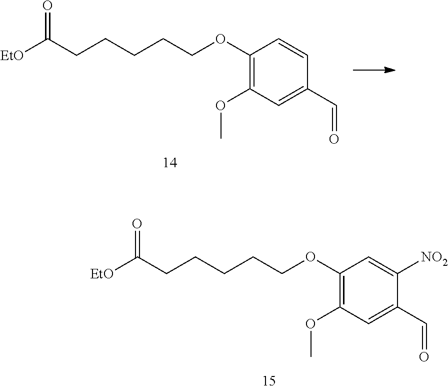 [SMILES: CCOC(=O)CCCCCOc1ccc(C=O)cc1OC ; CCOC(=O)CCCCCOc1cc([N+](=O)[O-])c(C=O)cc1OC]

A solution of potassium nitrate (5.40 g, 53.0 mmol) in trifluoroacetic acid (25 mL) at room temperature was charged slowly with a solution of ethyl 6-(4-formyl-2-methoxy-phenoxy)hexanoate (14) (12.5, 42.0 mmol) in trifluoroacetic acid (25 mL). The reaction mixture was stirred for 1 h and then concentrated in vacuo, after which the resulting residue was dissolved in ethyl acetate (200 mL). This was then washed with brine (3×50 mL) followed by a saturated aqueous solution of sodium hydrogen carbonate (2×40 mL). The organic extract was then dried over magnesium sulfate and concentrated in vacuo to give the title compound (14.4 g, 99%) as a yellow solid. This was carried through to the next step without any further purification.

¹H NMR (400 MHz, CDCl₃) δ 10.43 (s, 1H) 7.58 (s, 1H), 7.40 (s, 1H), 4.10-4.16 (m, 4H), 4.00 (s, 3H), 2.35 (t, J=7.4 Hz, 2H), 1.84-1.96 (m, 2H), 1.69-1.76 (m, 2H), 1.50-1.58 (m, 2H), 1.25 (t, J=70.2 Hz, 3H); MS M/Z (EIMS)=340 (M+H)⁺; LCMS (Method B): t_{R}=4.02 min.

### Example 18: 4-((6-Ethoxy-6-oxohexyl)oxy)-5-methoxy-2-nitrobenzoic acid (16)

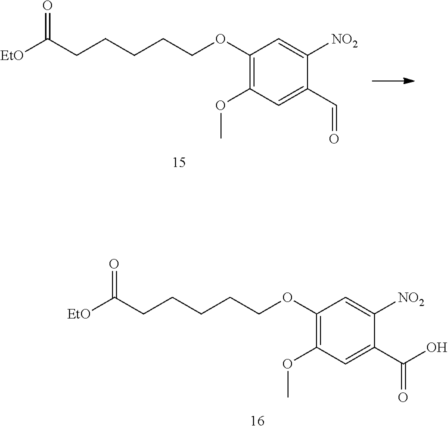 [SMILES: CCOC(=O)CCCCCOc1cc([N+](=O)[O-])c(C=O)cc1OC ; CCOC(=O)CCCCCOc1cc([N+](=O)[O-])c(C(=O)O)cc1OC]

A solution of ethyl 6-(4-formyl-2-methoxy-5-nitrophenoxy)hexanoate (15) (7.80 g, 23.0 mmol) in acetone (200 mL) was charged with a hot (70° C.) solution of potassium permanganate (13.6 g, 86.0 mmol) in water (100 m1). The resulting mixture was stirred at 70° C. for 4 h and then cooled to room temperature and filtered through a pad of celite. The filter cake was subsequently washed with hot water (100 mL). A solution of sodium bisulfite in hydrochloric acid (1 M, 100 mL) was added to the filtrate, which was then extracted with dichloromethane (2×200 mL). The combined organic extracts were dried over sodium sulfate and concentrated in vacuo to give the title compound (5.0 g, 61%) as a yellow solid which was used in the next step without further purification.

¹H NMR (400 MHz, CDCl₃) δ 7.34 (s, 1H), 7.14 (s, 1H), 3.96-4.03 (m, 4H), 3.84 (s, 3H), 2.24 (t, J=7.4 Hz, 2H), 1.70-1.77 (m, 2H), 1.55-1.62 (m, 2H), 1.39-1.45 (m, 2H), 1.13 (t, J=7.1 Hz, 3H); MS M/Z (EIMS)=354 (M−H)⁺; LCMS (Method B): t_{R}=3.63 min.

### Example 19: Ethyl (S)-6-(4-(2-(hydroxymethyl)piperidine-1-carbonyl)-2-methoxy-5-nitrophenoxy)hexanoate (17)

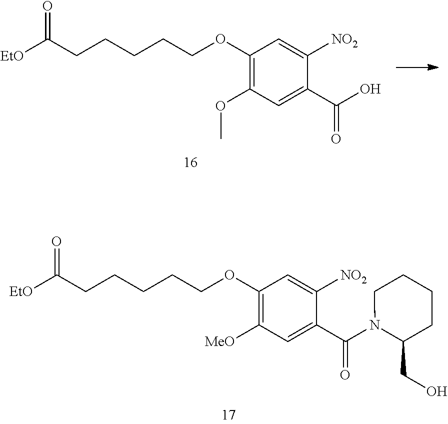 [SMILES: CCOC(=O)CCCCCOc1cc([N+](=O)[O-])c(C(=O)O)cc1OC ; CCOC(=O)CCCCCOc1cc([N+](=O)[O-])c(C(=O)N2CCCC[C@H]2CO)cc1OC]

A solution of 4-((6-ethoxy-6-oxohexyl)oxy)-5-methoxy-2-nitrobenzoic acid (16) (2.00 g, 5.60 mmol) in dichloromethane (40 mL) was charged with trimethylamine (4.70 mL, 33.8 mmol) and O-(7-azabenzotriazole-1-yl)-N,N,N,N′-tetramethyluronium hexafluoro-phosphate (2.20 g, 5.90 mmol) and the resulting mixture was stirred for 2 h at room temperature. A solution of (S)-piperidin-2-ylmethanol (647 mg, 5.63 mmol) in dichloromethane (10 mL) was then added and the resulting mixture was stirred for 16 h at room temperature. The reaction was quenched with a saturated aqueous solution of sodium hydrogen carbonate (40 mL), the phases were separated and the aqueous layer was further extracted with dichloromethane (20 mL). The combined organic extracts were washed with brine (40 mL), dried over magnesium sulfate, filtered and concentrated to give an amber oil. Purification was carried out by column chromatography (silica), eluting with ethyl acetate/hexane (from 0% to 100%), to give the title compound (1.20 g, 48%) as a colourless oil.

¹H NMR (400 MHz, CDCl₃) δ 7.63-7.60 (m, 1H), 6.77-6.75 (m, 1H), 4.13-4.02 (m, 4H), 3.93 (s, 3H), 3.78-3.70 (m, 1H), 3.68-3.39 (m, 1H), 3.18-3.11 (m, 3H), 2.32 (t, J=70.6 Hz, 2H), 1.91-1.83 (m, 2H), 1.72-1.39 (m, 11H), 1.26 (t, J=7.1 Hz, 3H); MS M/Z (EIMS)=453 (M+H)⁺; LCMS (Method B): t_{R}=3.63 min.

### Example 20: Ethyl (S)-6-(5-amino-4-(2-(hydroxymethyl)piperidine-1-carbonyl)-2-methoxyphenoxy)hexanoate (18)

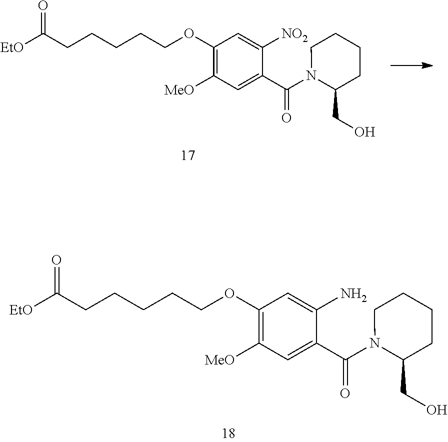 [SMILES: CCOC(=O)CCCCCOc1cc([N+](=O)[O-])c(C(=O)N2CCCC[C@H]2CO)cc1OC ; CCOC(=O)CCCCCOc1cc(N)c(C(=O)N2CCCC[C@H]2CO)cc1OC]

A solution of ethyl (S)-6-(4-(2-(hydroxymethyl)piperidine-1-carbonyl)-2-methoxy-5-nitrophenoxy) hexanoate (17) (1.20 g, 2.70 mmol) in methanol (20 mL) was charged with Raney®-Nickel (slurry in H₂O) (120 mg). The resulting mixture was hydrogenated at 4 atm for 1.5 h in a Parr apparatus, then filtered through a pad of celite and concentrated in vacuo to give the title compound (991 mg, 87%) as a grey oil that solidifies upon standing. The resulting material was carried through to the next step without further purification.

¹H NMR (400 MHz, CDCl₃) δ 6.69 (s, 1H), 6.32 (s, 1H), 4.13 (m, 4H), 3.98 (t, J=6.5 Hz, 2H), 3.79 (s, 3H), 3.67-3.57 (m, 1H), 3.22-3.19 (m, 4H), 2.87 (s, 2H), 2.36-2.32 (m, 2H), 1.89-1.82 (m, 2H), 1.73-1.65 (m, 6H), 1.55-1.47 (m, 3H), 1.27 (t, J=7.1 Hz, 3H); MS M/Z (EIMS)=423 (M+H)⁺; LCMS (Method B): t_{R}=3.23 min.

### Example 21: Ethyl (S)-6-(5-(((allyloxy)carbonyl)amino)-4-(2-(hydroxy-methyl)piperidine-1-carbonyl)-2-methoxyphenoxy)hexanoate (19)

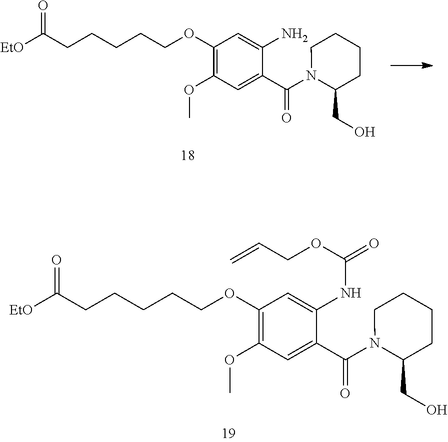 [SMILES: CCOC(=O)CCCCCOc1cc(N)c(C(=O)N2CCCC[C@H]2CO)cc1OC ; C=CCOC(=O)Nc1cc(OCCCCCC(=O)OCC)c(OC)cc1C(=O)N1CCCC[C@H]1CO]

A solution of ethyl (S)-6-(5-amino-4-(2-(hydroxymethyl)piperidine-1-carbonyl)-2-methoxyphenoxy) hexanoate (18) (1.23 g, 2.91 mmol) and anhydrous pyridine (542 μL, 6.69 mmol) in anhydrous dichloromethane (20 mL), at −10° C., was charged with a solution of allyl chloroformate (278 μL, 2.62 mmol) in dichloromethane (12 mL), dropwise. The resulting reaction mixture was stirred at room temperature for 0.5 h, quenched with a saturated aqueous solution of copper (II) sulfate (25 mL), diluted with dichloromethane (10 mL), separated and successively washed with water (20 mL), a saturated aqueous solution of sodium hydrogen carbonate (20 mL) and brine (20 mL). The organic extract was then dried over magnesium sulfate and concentrated in vacuo to give the title compound (588 mg, 40%) as an orange oil. The resulting material was carried through to the next step without further purification.

¹H NMR (400 MHz, CDCl₃) δ 8.23 (br s, 1H), 7.70 (br s, 1H), 6.78 (s, 1H), 6.00-5.90 (m, 1H), 5.38-5.33 (m, 1H), 5.24 (dd, J=10.4, 1.3 Hz, 1H), 4.63 (m, 2H), 4.12 (q, J=7.1 Hz, 2H) 4.05 (t, J=6.6 Hz, 2H), 3.83 (s, 3H), 3.72-3.64 (m, 1H), 3.12-3.02 (m, 1H), 2.33 (t, J=70.6 Hz, 2H), 1.91-1.84 (m, 2H), 1.74-1.67 (m, 10H), 1.66-1.54 (m, 4H), 1.26 (t, J=7.1 Hz, 3H); MS M/Z (EIMS)=507 (M+H)⁺; LCMS (Method B): t_{R}=3.70 min.

### Example 22: Allyl (6S,6aS)-3-((6-ethoxy-6-oxohexyl)oxy)-6-hydroxy-2-methoxy-12-oxo-6,6a,7,8,9,10-hexahydrobenzo[e]pyrido[1,2-a][1,4]-diazepine-5(12H)-carboxylate (20)

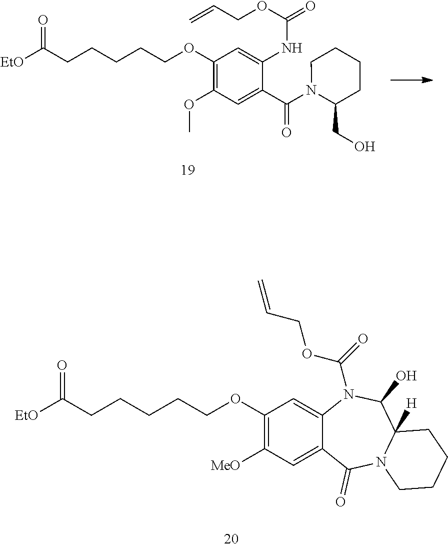 [SMILES: C=CCOC(=O)Nc1cc(OCCCCCC(=O)OCC)c(OC)cc1C(=O)N1CCCC[C@H]1CO ; C=CCOC(=O)N1c2cc(OCCCCCC(=O)OCC)c(OC)cc2C(=O)N2CCCC[C@H]2[C@@H]1O]

A solution of ethyl (S)-6-(5-(((allyloxy)carbonyl)amino)-4-(2-(hydroxymethyl)-piperidine-1-carbonyl)-2-methoxyphenoxy)hexanoate (19) (1.70 g, 3.40 mmol) in dichloromethane (80 mL) was charged with 2,2,6,6-tetramethyl-1-piperidinyloxy (53 mg, 0.30 mmol) and (diacetoxyiodo)benzene (1.30 g, 4.00 mmol). The resulting mixture was stirred at room temperature for 16 h and was then cooled in an ice bath and quenched with a saturated aqueous solution of sodium metabisulfite (35 mL). The mixture was then diluted with dichloromethane (30 mL), separated and sequentially washed with a saturated aqueous solution of sodium hydrogen carbonate (30 mL), water (30 mL) and brine (30 mL). The organic extract was then dried over magnesium sulfate and concentrated in vacuo. Purification was carried out by column chromatography (silica), eluting with ethyl acetate/hexane (from 0% to 80%) to give the desired compound (1.10 g, 66%) as a colourless oil.

¹H NMR (400 MHz, CDCl₃) δ 7.72-7.70 (m, 1H), 7.13-7.09 (m, 1H), 5.98-5.08 (m, 1H), 5.38-5.25 (m, 1H), 5.19-5.14 (m, 2H), 4.72-4.63 (m, 2H), 4.50-4.35 (m, 1H), 4.13 (q, J=7.1 Hz, 2H), 4.08-4.03 (m, 1H), 4.01-3.96 (m, 2H), 3.91 (s, 3H), 3.83-3.81 (m, 1H), 3.53-3.45 (m, 1H), 3.10-3.03 (m, 1H), 2.33 (t, J=70.6 Hz, 2H), 1.90-1.83 (m, 2H), 1.74-1.62 (m, 10H), 1.53-1.48 (m, 2H); MS M/Z (EIMS)=505 (M+H)⁺; LCMS (Method B): t_{R}=3.57 min.

### Example 23: Allyl (6S,6aS)-3-((6-ethoxy-6-oxohexyl)oxy)-2-methoxy-12-oxo-6-((tetrahydro-2H-pyran-2-yl)oxy)-6,6a,7,8,9,10-hexahydrobenzo[e]-pyrido[1,2-a][1,4]diazepine-5(12H)-carboxylate (21)

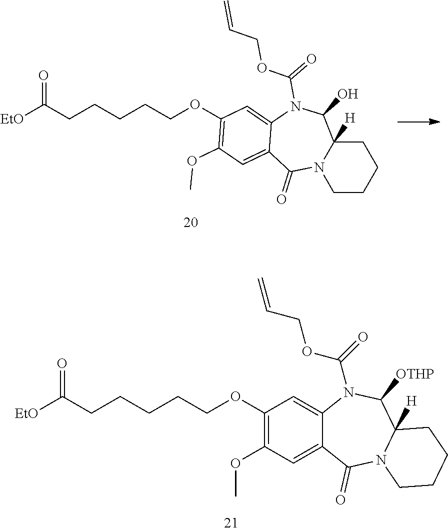

A solution of allyl (6S,6aS)-3-((6-ethoxy-6-oxohexyl)oxy)-6-hydroxy-2-methoxy-12-oxo-6,6a,7,8,9,10-hexahydrobenzo[e]pyrido[1,2-a][1,4]diazepine-5(12H)-carboxylate (20) (1.10 g, 2.20 mmol) in dichloromethane (50 mL) was charged with 3,4-dihydro-2H-pyran (2.00 mL, 22.4 mmol) and p-toluenesulfonic acid monohydrate (113 mg, 1% w/w). The resulting mixture was stirred at room temperature for 4 h. The reaction mixture was then diluted with dichloromethane (50 mL) and washed with a saturated aqueous solution of sodium hydrogen carbonate (50 mL) and brine (50 mL). The organic extract was then dried over magnesium sulfate and concentrated. Purification by column chromatography (silica), eluting with ethyl acetate/hexane (from 0% to 70%), gave the title compound (863 mg, 66%) as a yellow oil.

¹H NMR (400 MHz, CDCl₃) δ 7.16 (m, 1H), 6.50 (s, 1H), 6.10 (m, 1H), 5.81-5.76 (m, 1H), 5.14-5.03 (m, 2H), 4.69-4.57 (m, 2H), 4.47-4.37 (m, 1H), 4.34-4.26 (m, 1H), 4.12 (q, J=7.1 Hz, 2H), 4.01-3.94 (m, 3H), 3.90 (s, 3H), 3.68-3.62 (m, 1H), 3.68-3.46 (m, 2H), 3.12-3.03 (m, 1H), 2.33 (t, J=7.4 Hz, 2H), 1.89-1.66 (m, 11H), 1.57-1.47 (m, 6H), 1.25 (t, J=7.1 Hz, 3H); MS M/Z (EIMS)=589 (M+H)⁺; LCMS (Method B): t_{R}=4.32 min.

### Example 24: 6-(((6S,6aS)-5-((Allyloxy)carbonyl)-2-methoxy-12-oxo-6-((tetrahydro-2H-pyran-2-yl)oxy)-5,6,6a,7,8,9,10,12-octahydrobenzo[e]-pyrido[1,2-a][1,4]diazepin-3-yl)oxy)hexanoic acid (22)

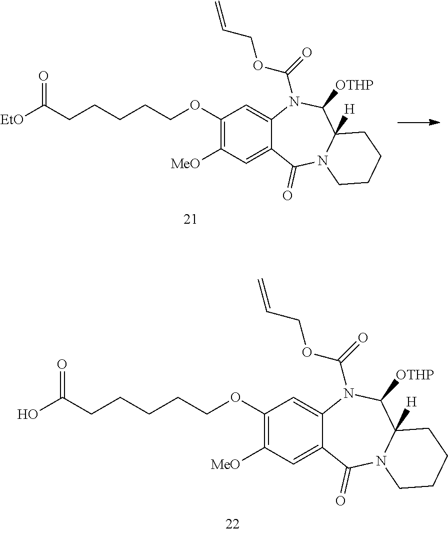

A solution of allyl (6S,6aS)-3-((6-ethoxy-6-oxohexyl)oxy)-2-methoxy-12-oxo-6-((tetrahydro-2H-pyran-2-yl)oxy)-6,6a,7,8,9,10-hexahydrobenzo[e]pyrido[1,2-a][1,4]-diazepine-5(12H)-carboxylate (21) (200 mg, 0.340 mmol) in 1,4-dioxane (3 m1) was charged with an aqueous solution of sodium hydroxide (0.5 M, 1.20 mL). The reaction mixture was stirred at room temperature for 2 h and was then concentrated in vacuo, after which water (6 m1) was added and the aqueous layer was then acidified to pH=1 with citric acid (1 M). The aqueous layer was then extracted with ethyl acetate (2×40 mL) and the combined organic extracts were then washed with brine (40 m1), dried over sodium sulfate and concentrated to give the title compound as a yellow oil (181 mg, 95%) which was used in the subsequent step without further purification.

¹H NMR (400 MHz, CDCl₃) δ 7.18 (s, 1H), 6.19 (s, 1H), 6.18-5.99 (m, 1H), 5.81-5.71 (m, 1H), 5.12-5.02 (m, 2H), 4.67-4.51 (m, 1H), 4.48-4.36 (m, 1H), 4.31-4.23 (m, 1H), 4.00-3.88 (m, 7H), 3.66-3.46 (m, 2H), 3.12-3.02 (m, 1H), 2.36 (t, J=7.4 Hz, 2H), 1.81-1.79 (m, 2H), 1.75-1.65 (m, 10H), 1.55-1.49 (m, 7H); MS M/Z (EIMS)=561 (M+H)⁺; LCMS (Method B): t_{R}=3.78 min.

### Example 25: Allyl (6S,6aS)-3-((6-((S)-1-(chloromethyl)-5-hydroxy-1,2-dihydro-3H-benzo[e]indol-3-yl)-6-oxohexyl)oxy)-2-methoxy-12-oxo-6-((tetrahydro-2H-pyran-2-yl)oxy)-6,6a,7,8,9,10-hexahydrobenzo[e]-pyrido[1,2-a][1,4]diazepine-5(12H)-carboxylate (23)

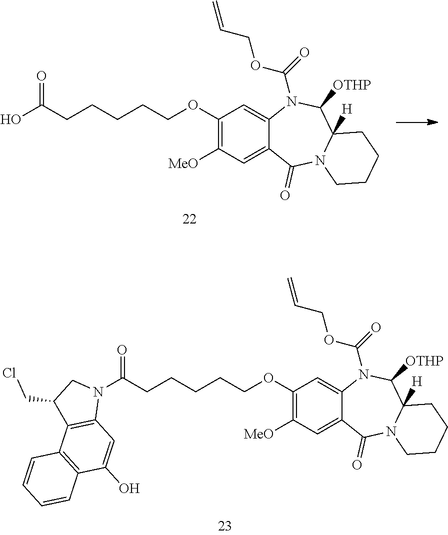

A solution of 6-(((6S,6aS)-5-((allyloxy)carbonyl)-2-methoxy-12-oxo-6-((tetrahydro-2H-pyran-2-yl)oxy)-5,6,6a,7,8,9,10,12-octahydrobenzo[e]pyrido[1,2-a][1,4]diazepin-3-yl)oxy)hexanoic acid (22) (37 mg, 0.066 mmol) in N,N-dimethylacetamide (1.0 mL) was charged with (S)-1-(chloromethyl)-2,3-dihydro-1H-benzo[e]indol-5-ol hydrochloride (11) (18 mg, 0.066 mmol) and N-(3-dimethylaminopropyl)-N′-ethylcarbodiimide hydrochloride (26.0 mg, 0.133 mmol) and stirred at room temperature under argon for 18 h. The reaction mixture was subsequently quenched with a saturated aqueous solution of sodium hydrogen carbonate, then taken up into ethyl acetate, separated and extracted with ethyl acetate (2×50 mL). The combined organic extracts were then washed with brine (50 mL), dried over magnesium sulfate and concentrated in vacuo. Column chromatography (silica), eluting with ethyl acetate/hexane (from 10% to 100%) followed by methanol (100%) afforded the title compound (11.4 mg, 22%) as a yellow oil.

¹H NMR (400 MHz, CDCl₃) δ 8.34 (d, J=70.2 Hz, 1H), 8.29 (d, J=8.2 Hz, 1H), 7.65 (d, J=8.4 Hz, 1H), 7.54-7.48 (m, 1H), 7.39-7.33 (m, 1H), 7.18 (s, 1H), 6.58 (s, 1H), 6.19 (d, J=10.0 Hz, 1H), 6.01 (d, J=10.0 Hz, 1H), 5.81-5.66 (m, 1H), 5.17-4.99 (m, 3H), 4.68-4.42 (m, 2H), 4.35-4.24 (m, 3H), 4.09-4.01 (m, 3H), 3.88 (s, 3H), 3.85-3.80 (m, 1H), 3.67-3.60 (m, 1H), 3.52-3.46 (m, 1H), 3.42 (t, J=11 Hz, 1H), 3.13-3.02 (m, 1H), 2.74-2.55 (m, 2H), 2.01-1.88 (m, 6H), 1.82-1.61 (m, 12H); ¹³C NMR (100 MHz, CDCl₃) δ 171.2, 163.7, 156.3, 155.1, 149.3, 146.6, 141.2, 131.6, 130.0, 127.6, 125.4, 123.9, 123.5, 122.7, 122.0, 117.9, 116.0, 114.6, 110.5, 108.0, 106.4, 100.4, 94.7, 69.1, 66.8, 63.0, 60.4, 56.1, 55.9, 52.3, 46.9, 46.3, 42.3, 35.7, 31.9, 29.7, 29.4, 25.5, 25.4, 25.2, 23.1, 22.7; MS M/Z (ES−)=774 (M−1)⁻; MS M/Z (EIMS)=798 (M+Na)⁺; LCMS (Method C): t_{R}=3.93 min.

### Example 26: (S)-3-((6-((S)-1-(Chloromethyl)-5-hydroxy-1,2-dihydro-3H-benzo[e]indol-3-yl)-6-oxohexyl)oxy)-2-methoxy-7,8,9,10-tetrahydrobenzo-[e]pyrido[1,2-a][1,4]diazepin-12(6aH)-one (24)

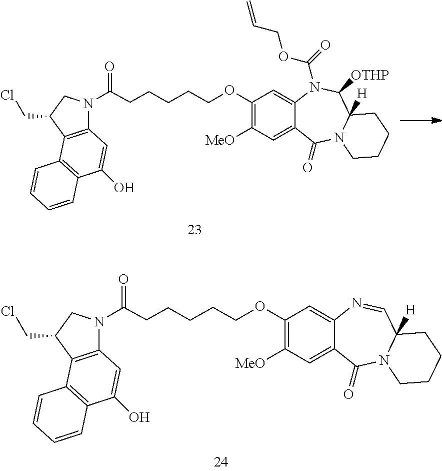

### Experiment (i)

A solution of allyl (6S,6aS)-3-((6-((S)-1-(chloromethyl)-5-hydroxy-1,2-dihydro-3H-benzo[e]indol-3-yl)-6-oxohexyl)oxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (23) (11 mg, 0.014 mmol) in dichloromethane (1 mL) was charged with tetrakis(triphenylphosphine)palladium(0) (1 mg) and pyrrolidine (10 μL) and then stirred at room temperature under argon. After approximately 1 min, the resulting mixture was concentrated in vacuo and immediately purified by column chromatography, eluting with ethyl acetate/hexane (from 50% to 100%) then with methanol/ethyl acetate (from 0% to 100%), to give the title compound (0.6 mg, 7.5%) as a yellow oil.

MS M/Z (EIMS)=590 (M+H)⁺; LCMS (Method C): t_{R}=3.80 min.

### Experiment (ii)

A solution of allyl (6S,6aS)-3-((6-((S)-1-(chloromethyl)-5-hydroxy-1,2-dihydro-3H-benzo[e]indol-3-yl)-6-oxohexyl)oxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (23) (27 mg, 0.035 mmol) in dichloromethane (4 mL) was charged with tetrakis(triphenylphosphine)palladium(0) (4 mg) and pyrrolidine (4 μL) and then stirred at room temperature under argon. After approximately 1 min, the resulting mixture was concentrated in vacuo and immediately purified by column chromatography, eluting with ethyl acetate/hexane (from 50% to 100%), to give the title compound (8 mg, 38%) as a yellow oil.

¹H NMR (400 MHz, acetone-d₆) δ 9.30 (br s, 1H), 8.21 (d, J=8.2 Hz, 1H), 8.13 (s, 1H), 7.97 (d, J=5.5 Hz, 1H), 7.80 (d, J=8.6 Hz, 1H), 7.74-7.67 (m, 1H), 7.63-7.49 (m, 1H), 7.33 (s, 1H), 6.78 (s, 1H), 4.39-4.30 (m, 2H), 4.19-4.11 (m, 3H), 4.10-4.05 (m, 1H), 4.01 (dd, J=11.0, 3.5 Hz, 1H), 3.85 (s, 3H), 3.79-3.68 (m, 2H), 3.16 (td, J=11.3, 3.1 Hz, 1H), 2.70-2.56 (m, 2H), 2.18-2.10 (m, 1H), 2.02-1.95 (m, 1H), 1.94-1.76 (m, 6H), 1.70-1.60 (m, 4H); MS (ES+): m/z=590 (M+H)⁺; LCMS (Method C): t_{R}=3.80 min, LCMS (Method A): t_{R}=7.20 min.

### Example 27: tert-Butyl (8bR,9aS)-4-oxo-9,9a-dihydro-1H-benzo[e]cyclo-propa[c]indole-2(4H)-carboxylate (25)

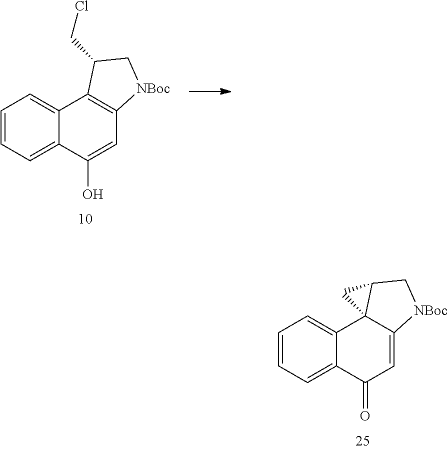 [SMILES: CC(C)(C)OC(=O)N1C[C@@H](CCl)c2c1cc(O)c1ccccc21 ; CC(C)(C)OC(=O)N1C[C@H]2C[C@@]23C1=CC(=O)c1ccccc13]

A solution of tert-butyl (S)-1-(chloromethyl)-5-hydroxy-1,2-dihydro-3H-benzo[e]-indole-3-carboxylate (10) (20 mg, 0.060 mmol) in anhydrous N,N-dimethylacetamide (1.0 mL) was cooled to 0° C. and charged with potassium carbonate (58.0 mg, 0.419 mmol) and stirred at this temperature for 25 min. The reaction mixture was then quenched (cold) with a saturated aqueous solution of sodium hydrogen carbonate and the resulting slurry extracted twice with ethyl acetate. The combined organic extracts were then dried over magnesium sulfate and concentrated in vacuo before purification was enacted by column chromatography (silica), eluting with ethyl acetate/hexane (25%, isocratic) to give the title compound (14 mg, 79%) as a yellow solid.

¹H NMR (400 MHz, CDCl₃) δ 8.22 (dd, J=70.9, 1.1 Hz, 1H), 7.49 (dd, J=70.7, 1.4 Hz, 1H), 7.39 (dt, J=70.6, 1.2 Hz, 1H), 6.86 (dd, J=70.8, 0.6 Hz, 1H), 6.82 (br s, 1H), 4.04-3.96 (m, 2H), 2.79-2.73 (m, 1H), 1.62 (dd, J=70.7, 4.4 Hz, 1H), 1.57 (s, 9H), 1.47 (t, J=4.7 Hz, 1H); ¹³C NMR (100 MHz, CDCl₃) δ 186.1, 159.7, 151.7, 140.2, 132.7, 131.8, 126.9, 126.5, 120.9, 108.7, 83.5, 52.9, 33.5, 28.2, 23.4, 14.1; MS M/Z (EIMS)=298 (M+H)⁺; LCMS (Method C): t_{R}=3.37 min.

General Synthetic Scheme to Prepare an a Group Precursor

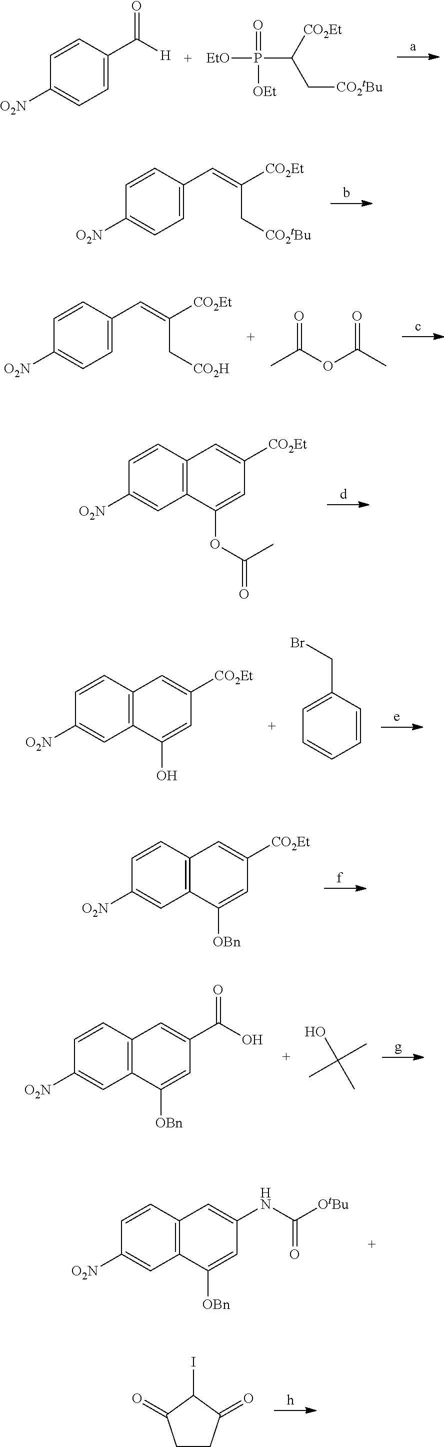

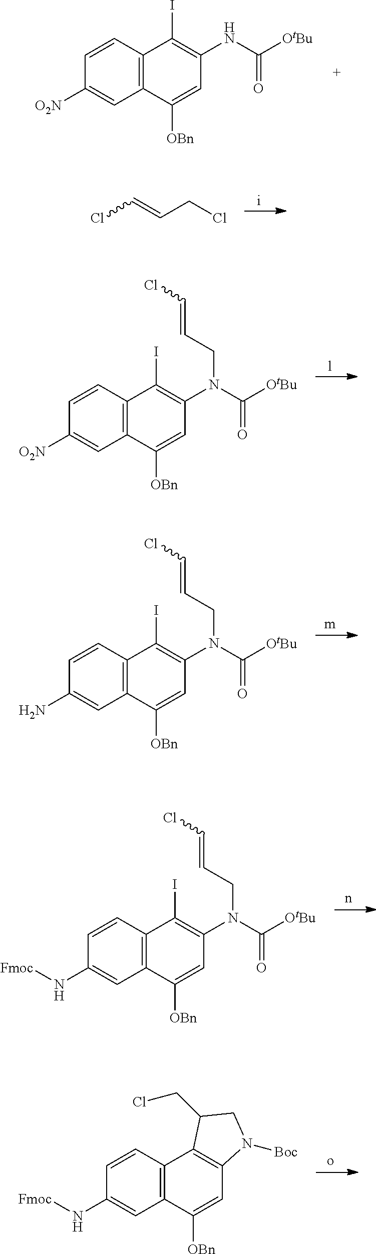

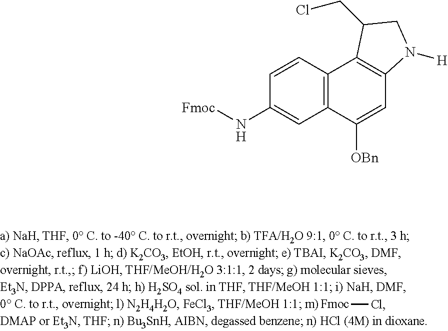

In step a, an aryl aldehyde is reacted with the phosphonate ester to produce an alkene compound. The tert-butyl protecting group is removed in step b to provide a carboxylic acid. In step c, this carboxylic acid is coupled with acetic anhydride to yield a naphthalene derivative. The acetyl group is removed in step d to produce the alcohol. In step d, the alcohol group is protected with a benzyl group. The ethyl ester is then removed in step f and the carboxylic acid is reacted with diphenylphosphoryl azide to produce an acyl azide that undergoes a Curtis rearrangement in the presence of tert-butanol to provide the tert-butyl carbamate in step g. The naphthalene ring is iodinated in step h to provide an aryl iodide derivative. In step i, the aryl carbamate is coupled with allyl chloride compound at the carbamate nitrogen. The aryl nitro group is reduced to the aryl amine in step 1, and this aryl amine is protected with a Fmoc protecting group in step m. Radical cyclisation is carried out in step n to preferentially provide the 5-membered ring exo cyclisation product. Removal of the Boc protecting group provides the A group precursor.

Further methods and experimental procedures for making A-rings and A-ring precursors have been disclosed by Jia and Lown (31) and by Elgersma et al. (32).

### Example 28: (S)-1-(Chloromethyl)-2,3-dihydro-1H-benzo[e]indol-5-ol hydrochloride (11)

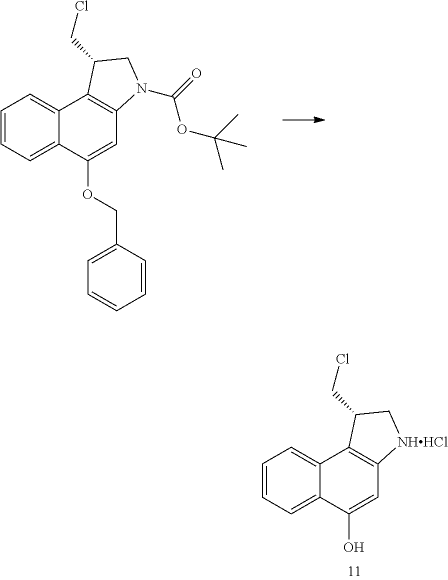

A solution of tert-butyl (S)-5-(benzyloxy)-1-(chloromethyl)-1,2-dihydro-3H-benzo[e]indole-3-carboxylate (100 mg, 0.236 mmol) in anhydrous dichloromethane (3 mL) was charged with boron trichloride (1 M solution in dichloromethane, 708 μL, 0.708 mmol), in a dropwise manner via syringe, at room temperature and under an inert atmosphere of argon. The resulting orange solution was stirred for 5 min before being quenched by cautious addition of methanol (5 mL), then concentrated in vacuo, charged again with methanol (5 mL) and re-concentrated to give the title compound (55 mg, quant.) as a pale green crystalline solid (unstable), which was used immediately in the subsequent step without further purification.

MS (ES+): m/z=234 (M+H)⁺; LCMS (Method C): t_{R}=2.62 min.

### Example 29: tert-Butyl (S)-1-(chloromethyl)-5-((4-methylpiperazine-1-carbonyl)oxy)-1,2-dihydro-3H-benzo[e]indole-3-carboxylate (26)

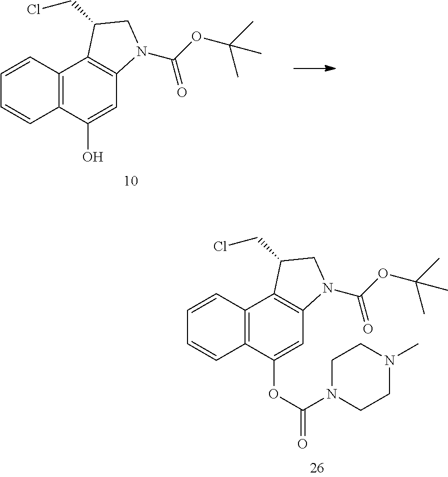 [SMILES: CC(C)(C)OC(=O)N1C[C@@H](CCl)c2c1cc(O)c1ccccc21 ; CN1CCN(C(=O)Oc2cc3c(c4ccccc24)[C@H](CCl)CN3C(=O)OC(C)(C)C)CC1]

A solution of tert-butyl (S)-1-(chloromethyl)-5-hydroxy-1,2-dihydro-3H-benzo[e]indole-3-carboxylate (10) (50 mg, 0.15 mmol) in dichloromethane (5 mL) was charged with 4-methyl-1-piperazinecarbonyl chloride hydrochloride (89 mg, 0.45 mmol), 4-(dimethylamino)pyridine (20 mg, 0.17 mmol) and triethylamine (73 μL, 0.52 mmol) and stirred at room temperature for 18 h. The reaction mixture was subsequently washed with water (2×10 mL), dried over magnesium sulfate and concentrated in vacuo, to give the title compound (59 mg, 86%) as a yellow solid.

¹H NMR (400 MHz, CDCl₃) δ 8.05 (br s, 1H), 7.82 (d, J=8.4 Hz, 1H), 7.68 (d, J=8.3 Hz, 1H), 7.51-7.45 (m, 1H), 7.38-7.33 (m, 1H), 4.28-4.21 (br, 1H), 4.15-4.07 (m, 1H), 4.03-3.96 (m, 1H), 3.94-3.88 (m, 1H), 3.72 (t, J=4.9 Hz, 2H), 3.68-3.60 (m, 2H), 3.45 (t, J=10.8 Hz, 1H), 2.61-2.50 (m, 4H), 2.31 (s, 3H), 1.57 (s, 9H); ¹³C NMR (100 MHz, CDCl₃) δ 153.4, 152.4, 148.4, 148.3, 130.2, 127.6, 124.2, 124.1, 122.6, 122.3, 120.1, 109.3, 81.2, 54.6, 54.2, 48.5, 46.3, 46.1, 45.8, 28.4; MS (ES+): m/z=460 (M+H)⁺; LCMS (Method C): t_{R}=3.00 min.

Reaction scheme for preparing compound (29)

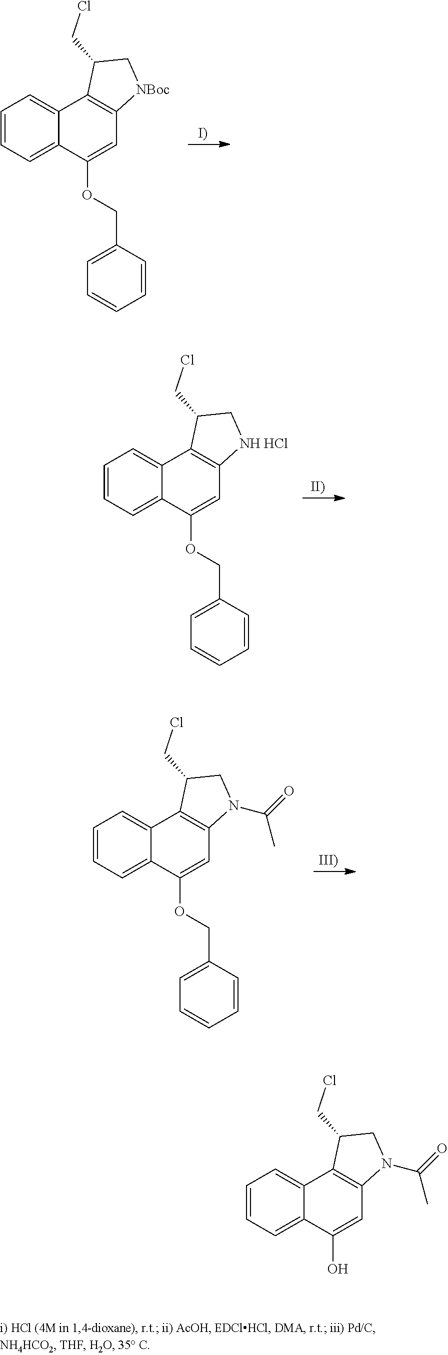

### Example 30: (S)-5-(Benzyloxy)-1-(chloromethyl)-2,3-dihydro-1H-benzo[e]indole hydrochloride (27)

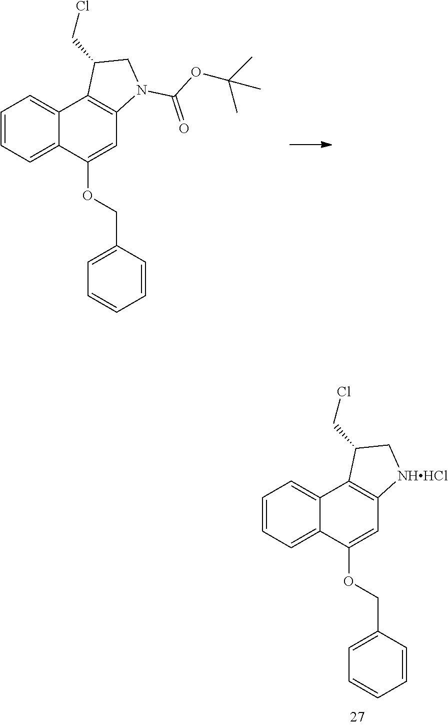

A solution of tert-butyl (S)-5-(benzyloxy)-1-(chloromethyl)-1,2-dihydro-3H-benzo[e]indole-3-carboxylate (100 mg, 0.236 mmol) in 1,4-dioxane (1 mL) was charged with hydrochloric acid (4 M in 1,4-dioxane) (2 mL) dropwise and stirred at room temperature for 2 h, whereupon it was concentrated in vacuo to give the title compound (85 mg, quant.) as a green solid (unstable), which was used immediately in the subsequent step without further purification.

MS (ES+): m/z=324 (M+H)⁺; LCMS (Method C): t_{R}=3.77 min.

### Example 31: (S)-1-(5-(Benzyloxy)-1-(chloromethyl)-1,2-dihydro-3H-benzo-[e]indol-3-yl)ethan-1-one (6)

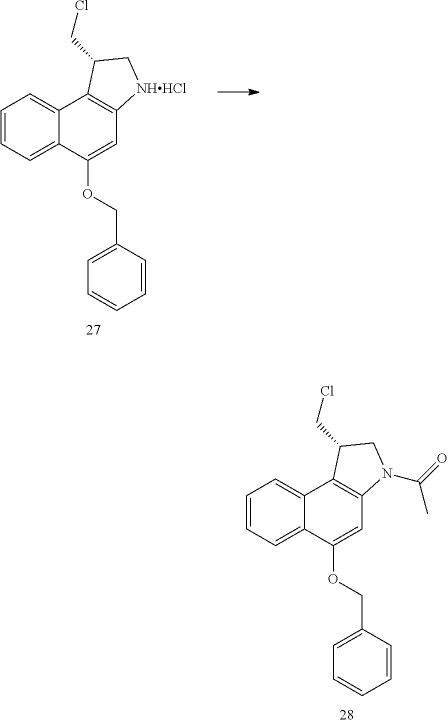

A solution of (S)-5-(benzyloxy)-1-(chloromethyl)-2,3-dihydro-1H-benzo[e]indole hydrochloride (27) (85 mg, 0.24 mmol) in N,N-dimethylacetamide (1 mL) was charged with acetic acid (100 μL), and N-(3-dimethylaminopropyl)-N′-ethylcarbodiimide hydrochloride (45 mg, 0.24 mmol) and stirred at room temperature for 18 h. The reaction mixture was subsequently quenched with a saturated aqueous solution of sodium hydrogen carbonate, and extracted with ethyl acetate (2×50 mL). The combined organic extracts were then washed with brine (50 mL), dried over magnesium sulfate and concentrated in vacuo. Column chromatography (silica), eluting with ethyl acetate/hexane (from 0% to 20%) afforded the title compound (53 mg, 62%) as a white solid.

¹H NMR (400 MHz, CDCl₃) S8.34 (d, J=8.4 Hz, 1H), 8.18 (s, 1H), 7.67 (d, J=8.3 Hz, 1H), 7.58-7.53 (m, 3H), 7.46-7.41 (m, 2H), 7.40-7.35 (m, 2H), 5.30 (dd, J=11.8, 2.0 Hz, 2H), 4.28 (br, 1H), 4.08-4.01 (m, 1H), 3.98 (dd, J=11.2, 3.0 Hz, 1H), 3.80-3.63 (m, 1H), 3.47-3.41 (m, 1H), 2.33 (s, 3H); ¹³C NMR (100 MHz, CDCl₃) δ 169.1, 155.9, 141.7, 136.8, 129.8, 128.6, 128.0, 127.6, 123.7, 123.7, 123.3, 122.0, 115.2, 110.3, 98.0, 70.4, 54.0, 46.2, 42.4, 22.7; MS (ES+): m/z=366 (M+H)⁺; LCMS (Method C): t_{R}=4.38 min.

### Example 32: (S)-1-(1-(Chloromethyl)-5-hydroxy-1,2-dihydro-3H-benzo[e]indol-3-yl)ethan-1-one (7)

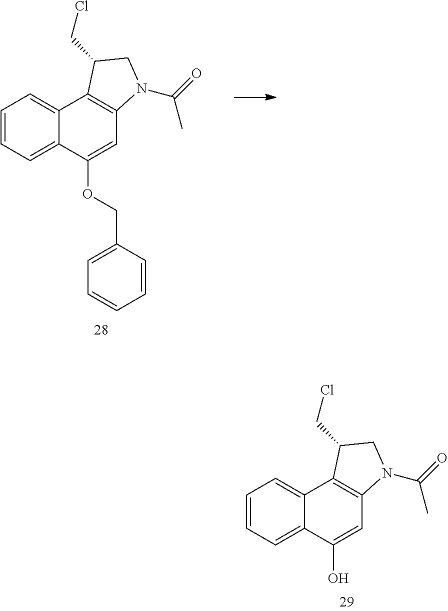 [SMILES: CC(=O)N1C[C@@H](CCl)c2c1cc(OCc1ccccc1)c1ccccc21 ; CC(=O)N1C[C@@H](CCl)c2c1cc(O)c1ccccc21]

A solution of (S)-1-(5-(benzyloxy)-1-(chloromethyl)-1,2-dihydro-3H-benzo[e]indol-3-yl)ethan-1-one (28) (24 mg, 0.066 mmol) in tetrahydrofuran (1 mL) was charged with ammonium formate (25% aqueous solution) (132 μL, 0.53 mmol) and palladium on activated charcoal (10 wt. % basis) (2 mg) and then heated to 35° C. under an inert atmosphere of argon. After 3 h, the mixture was allowed to cool and filtered through a pad of celite, washed with acetone and then concentrated in vacuo. After diluting in ethyl acetate and washing with water (50 mL) followed by brine (50 mL), the organic extract was dried over magnesium sulfate and concentrated in vacuo to give the title compound (6.3 mg, 35%) as a green solid.

¹H NMR (400 MHz, CDCl₃) S8.47 (s, 1H), 8.32 (d, J=8.1 Hz, 1H), 7.69 (d, J=8.3 Hz, 1H), 7.48 (td, J=70.6, 1.3 Hz, 1H), 7.38 (m, 1H), 4.24-4.16 (m, 1H), 3.80-3.69 (m, 4H), 2.38 (s, 3H); MS (ES+): m/z=276 (M+H)⁺; LCMS (Method C): t_{R}=3.18 min.

### Example 3: Ethyl 2-(3-(bromomethyl)phenyl)acetate (30)

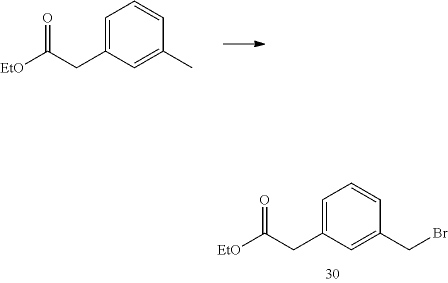

A mixture of N-bromosuccinimide (12.5 g, 71.2 mmol), azobisisobutyronitrile (366 mg, 2.30 mmol) and ethyl m-tolylacetate (10 mL, 56.6 mmol) in carbon tetrachloride (60 mL) was stirred at reflux for 3 h. The reaction mixture was then allowed to cool to room temperature, filtered, and the filtrate concentrated in vacuo. Purification by column chromatography (silica), eluting with ethyl acetate/hexane (from 0% to 9%) gave the title compound (6.7 g, 46%) as a colourless oil.

¹H NMR (400 MHz, CDCl₃) δ 7.34-7.23 (m, 4H), 4.50 (s, 2H), 4.22-4.13 (m, 2H), 3.63 (s, 2H), 1.29 (t, J=8.2 Hz, 3H); ¹³C NMR (100 MHz, CDCl₃) δ 171.3, 138.1, 134.7, 129.9, 129.3, 129.0, 128.7, 60.9, 41.1, 33.3, 14.2; MS (ES+): m/z=258 (M+H)⁺; LCMS (Method B): t_{R}=3.90 min.

Reaction scheme for preparing compound (42)

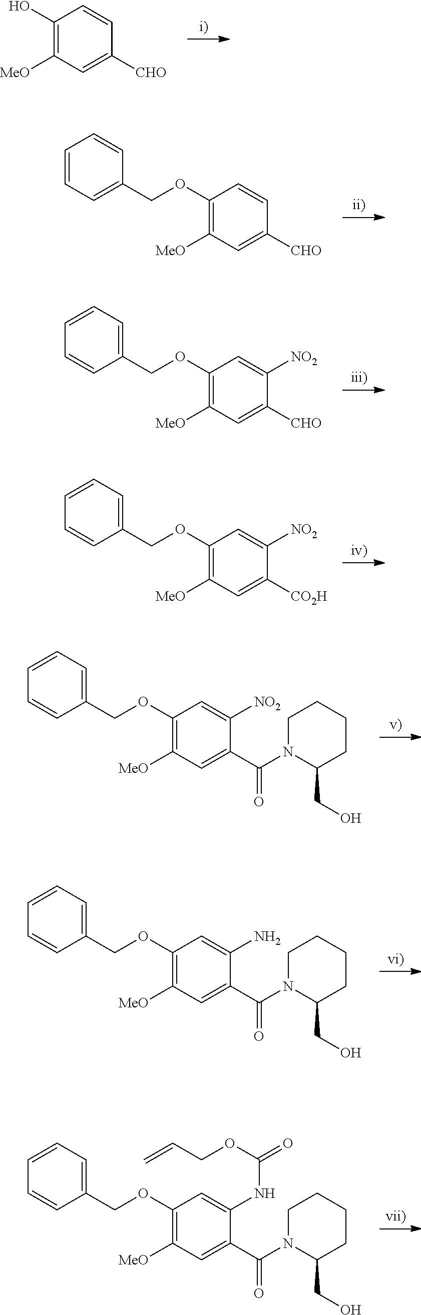

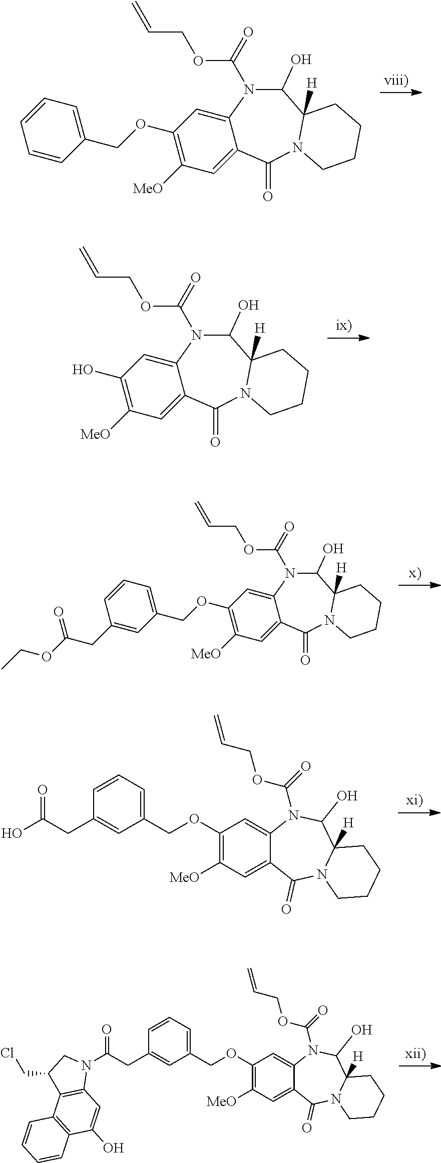 [SMILES: C=CCOC(=O)N1c2cc(OCc3ccccc3)c(OC)cc2C(=O)N2CCCC[C@H]2C1O ; C=CCOC(=O)N1c2cc(O)c(OC)cc2C(=O)N2CCCC[C@H]2C1O ; C=CCOC(=O)N1c2cc(OCc3cccc(CC(=O)OCC)c3)c(OC)cc2C(=O)N2CCCC[C@H]2C1O ; C=CCOC(=O)N1c2cc(OCc3cccc(CC(=O)O)c3)c(OC)cc2C(=O)N2CCCC[C@H]2C1O ; C=CCOC(=O)N1c2cc(OCc3cccc(CC(=O)N4C[C@@H](CCl)c5c4cc(O)c4ccccc54)c3)c(OC)cc2C(=O)N2CCCC[C@H]2C1O]

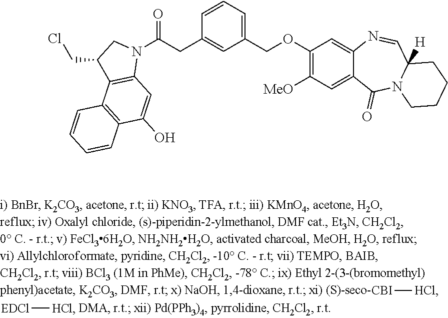

### Example 34: 4-(Benzyloxy)-3-methoxybenzaldehyde (31)

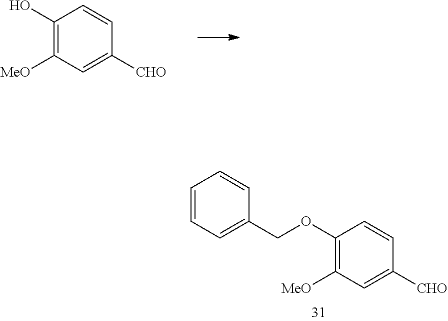 [SMILES: COc1cc(C=O)ccc1O ; COc1cc(C=O)ccc1OCc1ccccc1]

Method (i)—A mixture of vanillin (15.0 g, 99 mmol), benzyl bromide, (12.9 mL, 109 mmol) and potassium carbonate (6.7 g, 0.49 mmol) in acetone (225 mL) was stirred at room temperature for 18 h. The reaction was diluted with water (200 mL) and extracted with ethyl acetate (2×200 mL). The combined organics were then washed with water (100 mL) and brine (100 mL), dried over magnesium sulfate, filtered and concentrated to give the title compound (12.5 g, 62%) as a pale yellow solid. The product was carried through to the next step without further purification.

¹H NMR (400 MHz, CDCl₃) δ 9.84 (s, 1H), 7.45-7.31 (m, 7H), 6.98 (d, J=8.2 Hz, 1H), 5.24 (s, 2H), 3.94 (s, 3H); ¹³C NMR (100 MHz, CDCl₃) δ 190.9, 153.6, 150.1, 136.0, 130.3, 128.7, 128.2, 127.2, 126.6, 112.4, 109.4, 70.9, 56.0; MS (ES+): m/z=243 (M+H)⁺, MS (ES−): m/z=241 (M−1)⁻; LCMS (Method B): t_{R}=3.82 min; LCMS (Method A): t_{R}=7.53 min.

Method (ii)—A mixture of compound vanillin (200 g, 1.31 mol), benzyl bromide (236 g, 1.38 mol) and potassium carbonate (545 g, 3.94 mol) in methanol (1.20 L) was refluxed for 5 h. The reaction mixture was filtered, and the filtrate evaporated under reduced pressure to afford the title compound (271 g, 85%) as a pale yellow solid.

¹H NMR (400 MHz, CDCl₃) δ 9.83 (s, 1H), 7.47-7.35 (m, 6H), 7.33 (d, J=70.2 Hz, 1H), 6.98 (d, J=8.2 Hz, 1H), 5.24 (s, 2H), 3.94 (s, 3H); ¹³C NMR (100 MHz, CDCl₃) δ 191.0, 153.6, 150.1, 136.0, 130.3, 128.7, 128.2, 127.2, 126.6, 112.3, 109.3, 70.9, 56.1; MS (ES+): m/z=243 (M+H)⁺; LCMS (Method A): t_{R}=7.53 min.

### Example 5:4-(Benzyloxy)-5-methoxy-2-nitrobenzaldehyde (12)

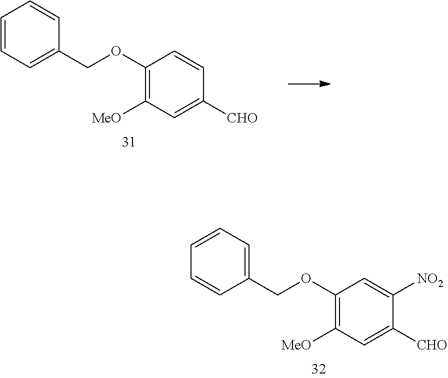 [SMILES: COc1cc(C=O)ccc1OCc1ccccc1 ; COc1cc(C=O)c([N+](=O)[O-])cc1OCc1ccccc1]

Method (i)—A solution of potassium nitrate (5.4 g, 53 mmol) in trifluoroacetic acid (25 mL) was added dropwise to a solution of 4-(benzyloxy)-3-methoxybenzaldehyde (31) (12.5 g, 42 mmol) in trifluoroacetic acid (25 mL) at room temperature. The reaction mixture was stirred for 1 h. It was then concentrated in vacuo and the residue was dissolved in ethyl acetate (200 mL). The organic layer was successively washed with brine (3×50 mL) and a saturated aqueous solution of sodium hydrogen carbonate (2×40 mL), dried over magnesium sulfate, filtered and concentrated to give the title compound (14.4 g, 100%) as a yellow solid. The product was used in the next step without further purification.

¹H NMR (400 MHz, DMSO-d₆) δ 10.21 (s, 1H), 7.84 (s, 1H), 7.50-7.38 (m, 6H), 5.33 (s, 2H), 3.96 (s, 3H); ¹³C NMR (100 MHz, CDCl₃) δ 188.6, 152.8, 150.8, 135.6, 128.6, 128.5, 128.3, 128.1, 124.9, 110.2, 108.7, 70.7, 56.5; MS (ES+): m/z=288 (M+H)⁺, MS (ES−): m/z=286 (M−1)⁻; LCMS (Method B): t_{R}=3.98 min, LCMS (Method A): t_{R}=7.67 min.

Method (ii)—A solution of 4-(benzyloxy)-3-methoxybenzaldehyde (31) (130 g, 537 mmol) in trifluoroacetic acid (600 mL) was charged with a solution of potassium nitrate (65 g, 644 mmol), in trifluoroacetic acid (600 mL) dropwise at ° C. The reaction mixture was stirred for 1 h and then diluted with water (2.40 L). The resulting precipitate was filtered and washed with cold water (500 mL×2) to afford the title compound (125 g, 81%) as a yellow solid.

¹H NMR (400 MHz, CDCl₃) δ 10.43 (s, 1H), 7.67 (s, 1H), 7.46-7.30 (m, 6H), 5.27 (s, 2H), 4.02 (s, 3H); ¹³C NMR (100 MHz, CDCl₃) δ 187.8, 153.7, 151.4, 134.85, 129.0, 128.9, 128.7, 127.6, 125.7, 110.0, 108.9, 71.6, 56.7; MS (ES−): m/z=286 (M−H)⁻; LCMS (Method A): t_{R}=7.87 min.

### Example 36: 4-(Benzyloxy)-5-methoxy-2-nitrobenzoic acid (33)

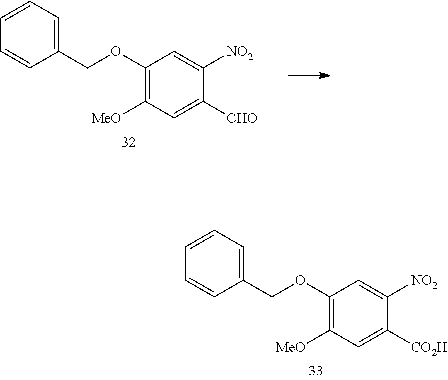 [SMILES: COc1cc(C=O)c([N+](=O)[O-])cc1OCc1ccccc1 ; COc1cc(C(=O)O)c([N+](=O)[O-])cc1OCc1ccccc1]

A solution of 4-(benzyloxy)-5-methoxy-2-nitrobenzaldehyde (32) (8.0 g, 28 mmol) in acetone (300 mL) was quickly charged with a hot (70° C.) solution of potassium permanganate (16.5 g, 104 mmol) in water (150 m1). The mixture was then stirred at 70° C. for 4 h. The reaction mixture was then allowed to cool to room temperature and passed through a pad of celite, which was then washed with hot water (120 mL). A solution of sodium bisulfite in hydrochloric acid (1 M, 120 mL) was added to the filtrate, which was then extracted with dichloromethane (2×200 mL). The combined organic extracts were subsequently dried over sodium sulfate, filtered and concentrated to give the title compound (6.7 g, 79%) as a yellow solid, which was used in the subsequent step without further purification.

¹H NMR (400 MHz, CDCl₃) δ 7.55 (s, 1H), 7.49-7.37 (m, 6H), 5.17 (s, 2H), 4.99 (br s, 1H), 3.93 (s, 3H); ¹³C NMR (100 MHz, MeOD) δ 168.6, 154.1, 151.0, 142.9, 137.3, 129.7, 129.4, 129.0, 123.2, 112.5, 110.0, 72.3, 57.1; MS (ES+): m/z=302 (M+H)⁺, MS (ES−): m/z=302 (M−1)⁻; LCMS (Method B): t_{R}=3.62 min, LCMS (Method A): t_{R}=7.02 min.

### Example 37: (S-(4-(Benzyloxy)-5-methoxy-2-nitrophenyl)(2-(hydroxy-methyl)piperidin-1-yl)methanone (34)

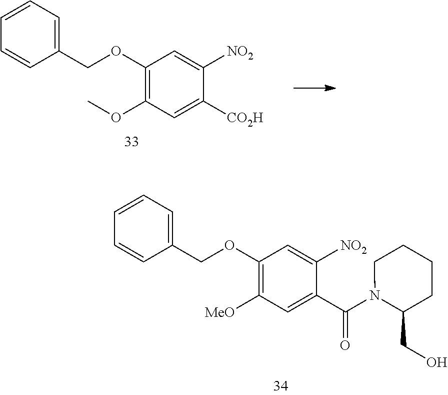 [SMILES: COc1cc(C(=O)O)c([N+](=O)[O-])cc1OCc1ccccc1 ; COc1cc(C(=O)N2CCCC[C@H]2CO)c([N+](=O)[O-])cc1OCc1ccccc1]

A solution of 4-(benzyloxy)-5-methoxy-2-nitrobenzoic acid (33) (1.00 g, 3.30 mmol) and oxalyl chloride (0.84 mL, 9.90 mmol) in anhydrous dichloromethane (10 mL) was charged with N,N-dimethylformamide (drops) at 0° C. The resulting mixture was stirred for 2 h at room temperature, and then concentrated in vacuo. Anhydrous toluene was then charged to the resulting residue and the mixture concentrated again.

After re-solubilising in anhydrous dichloromethane (10 mL), the resulting solution was then added dropwise to a solution of (S)-piperidin-2-ylmethanol (494 mg, 4.30 mmol) and triethylamine (1.4 mL, 9.9 mmol) in anhydrous dichloromethane (10 mL). The resulting mixture was stirred for 16 h at room temperature. The reaction was quenched with hydrochloric acid (1 M, 20 mL), the phases were separated and the organic extract was washed with brine (15 mL), dried over sodium sulfate, filtered and concentrated in vacuo. Purification was carried out by column chromatography (silica), eluting with ethyl acetate/hexane (from 0% to 50%), to give the title compound (974 mg, 74%) as an amber oil.

¹H NMR (400 MHz, CDCl₃) δ 7.76 (s, 1H), 7.44-7.38 (m, 5H), 6.83 (s, 1H), 5.20 (s, 2H), 4.37 (br s, 1H), 3.98 (s, 3H), 3.94-3.78 (m, 4H), 3.16 (m, 2H), 2.19-1.83 (m, 5H); MS (ES+): m/z=401 (M+H)⁺; LCMS (Method B): t_{R}=3.60 min.

### Example 38: (S)-(2-Amino-4-(benzyloxy)-5-methoxyphenyl)(2-(hydroxy-methyl)piperidin-1-yl)methanone (35)

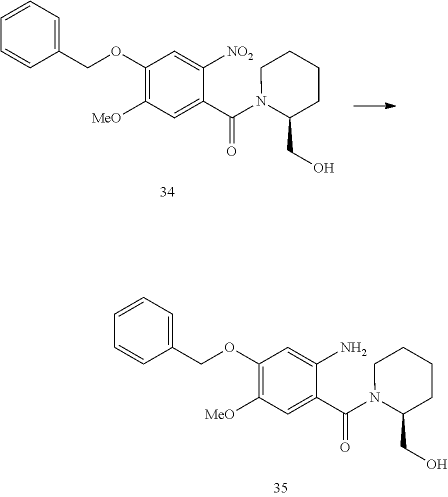 [SMILES: COc1cc(C(=O)N2CCCC[C@H]2CO)c([N+](=O)[O-])cc1OCc1ccccc1 ; COc1cc(C(=O)N2CCCC[C@H]2CO)c(N)cc1OCc1ccccc1]

A solution of(S)-(4-(benzyloxy)-5-methoxy-2-nitrophenyl)(2-(hydroxymethyl)-piperidin-1-yl)methanone (34) (1.67 g, 4.18 mmol) in methanol (60 mL) and water (60 mL) was sequentially charged with activated charcoal (2.26 g, 188 mmol), iron(III) chloride hexahydrate (678 mg, 2.51 mmol) and hydrazine monohydrate (2.51 mL, 50.2 mmol) under an inert atmosphere of nitrogen. The reaction mixture was then heated to reflux for 16 h, before cooling to room temperature, filtering through a pad of celite and concentrating in vacuo. After extracting with ethyl acetate (2×80 mL), the organic extracts were combined, washed with brine (100 m1), dried over sodium sulfate, filtered and concentrated in vacuo. Purification by column chromatography (silica), eluting with ethyl acetate/hexane (from 0 to 100%) gave the title compound (991 mg, 82%) as a yellow oil that solidifies upon standing.

MS (ES+): m/z=371 (M+H)⁺, MS (ES−): m/z=369 (M−1)⁻; LCMS (Method B): t_{R}=3.22 min.

### Example 39: Allyl (S)-(5-(benzyloxy)-2-(2-(hydroxymethyl)piperidine-1-carbonyl)-4-methoxyphenyl)carbamate (36)

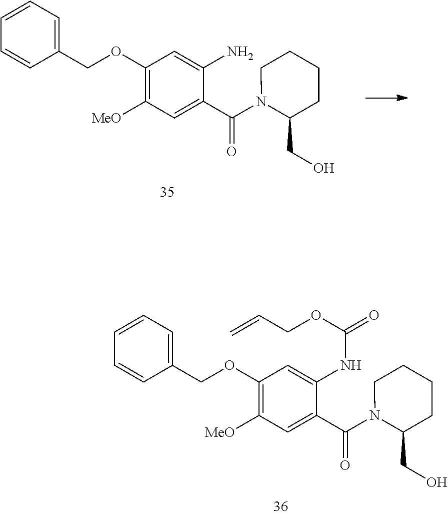 [SMILES: COc1cc(C(=O)N2CCCC[C@H]2CO)c(N)cc1OCc1ccccc1 ; C=CCOC(=O)Nc1cc(OCc2ccccc2)c(OC)cc1C(=O)N1CCCC[C@H]1CO]

A solution of(S)-(2-amino-4-(benzyloxy)-5-methoxyphenyl)(2-(hydroxymethyl)-piperidin-1-yl)methanone (35) (942 mg, 2.54 mmol) and pyridine (473 μL, 5.48 mmol) in anhydrous dichloromethane (10 mL) at −10° C., was slowly charged with a solution of allylchloroformate (243 μL, 2.29 mmol) in dichloromethane (10 mL). The resulting mixture was stirred at room temperature for 0.5 h, before diluting with dichloro-methane (10 mL) and extracting with a saturated aqueous solution of copper (II) sulfate (25 mL). The organic phase was then washed successively with water (20 mL), a saturated aqueous solution of sodium hydrogen carbonate (20 mL) and brine (20 mL), then dried over sodium sulfate, filtered and concentrated in vacuo. Purification by column chromatography (silica), eluting with ethyl acetate/hexane (from 0 to 100%) gave the title compound (789 mg, 68%) as a colourless oil that solidifies upon standing.

¹H NMR (400 MHz, CDCl₃) δ 8.30 (br s, 1H), 7.80 (br s, 1H), 7.49-7.47 (m, 2H) 7.40-7.30 (m, 3H) 6.82 (brs, 1H), 6.00-5.91 (m, 1H), 5.39-5.34 (m, 1H), 5.24 (dd, J=10.4, 1.3 Hz, 1H), 5.16 (s, 2H), 4.64 (dd, J=5.4, 1.3 Hz, 2H), 3.98-3.90 (m, 1H), 3.85 (s, 3H), 3.71-3.57 (m, 2H), 3.25-2.98 (m, 2H), 1.79-1.63 (m, 4H), 1.58-1.44 (m, 2H); MS (ES+): m/z=455 (M+H)⁺, MS (ES−): m/z=453 (M−1)⁻; LCMS (Method B): t_{R}=3.72 min.

### Example 40: Allyl (6aS)-3-(benzyloxy)-6-hydroxy-2-methoxy-12-oxo-6,6a,7,8,9,10-hexahydrobenzo[e]pyrido[1,2-a][1,4]diazepine-5(12H)-carboxylate (37)

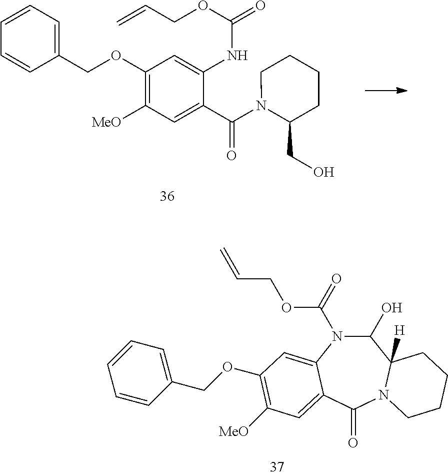 [SMILES: C=CCOC(=O)Nc1cc(OCc2ccccc2)c(OC)cc1C(=O)N1CCCC[C@H]1CO ; C=CCOC(=O)N1c2cc(OCc3ccccc3)c(OC)cc2C(=O)N2CCCC[C@H]2C1O]

A solution of allyl (S)-(5-(benzyloxy)-2-(2-(hydroxymethyl)piperidine-1-carbonyl)-4-methoxyphenyl)carbamate (36) (789 mg, 1.74 mmol) in dichloromethane (20 mL) was charged with 2,2,6,6-tetramethyl-1-piperidinyloxy (28 mg, 0.17 mmol) and (diacetoxyiodo)benzene (672 mg, 2.10 mmol). The reaction mixture was stirred at room temperature for 16 h and then placed in an ice bath before quenching with a saturated aqueous solution of sodium metabisulfite (15 mL). After extracting with dichloro-methane (20 mL), the organic layer was sequentially washed with a saturated aqueous solution of sodium hydrogen carbonate (20 mL), water (20 mL) and brine (20 mL), then dried over sodium sulfate, filtered and concentrated in vacuo. Purification was carried out by column chromatography (silica), eluting with ethyl acetate/hexane (from 0% to 100%) to give the title compound (347 mg, 44%) as a colourless oil.

¹H NMR (400 MHz, CDCl₃) δ 7.43-7.29 (m, 5H), 7.20 (s, 1H), 6.69 (br s, 1H), 5.90 (d, J=10.3 Hz, 1H), 5.29 (s, 2H), 5.17-5.05 (m, 4H), 4.50 (br s, 1H), 4.44 (br s, 1H), 4.41-4.31 (m, 1H), 3.92 (s, 3H), 3.48 (ddd, J=10.2, 6.0, 3.8 Hz, 1H), 3.11-3.00 (m, 1H), 2.07-1.99 (m, 1H), 1.82-1.55 (m, 5H); ¹³C NMR (100 MHz, CDCl₃) δ 207.1, 168.9, 156.1, 150.0, 149.2, 136.3, 131.9, 128.6, 128.1, 127.3, 125.6, 118.0, 114.2, 110.8, 82.4, 71.1, 66.7, 56.2, 55.3, 38.7, 30.9, 23.2, 23.0; MS (ES+): m/z=453 (M+H)⁺, MS (ES−): m/z=451 (M−1)⁻; LCMS (Method B): t_{R}=3.53 min.

### Example 41: Allyl (6aS)-3,6-dihydroxy-2-methoxy-12-oxo-6,6a,7,8,9,10-hexahydrobenzo[e]pyrido[1,2-a][1,4]diazepine-5(12H)-carboxylate (38)

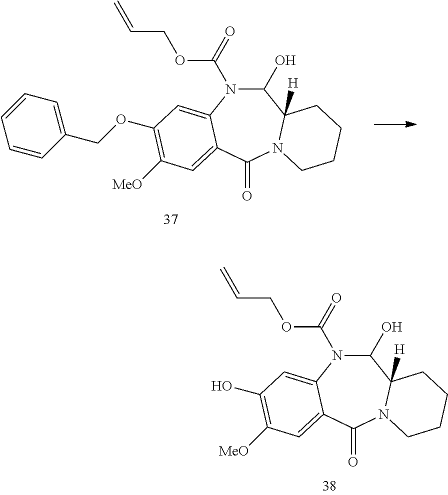 [SMILES: C=CCOC(=O)N1c2cc(OCc3ccccc3)c(OC)cc2C(=O)N2CCCC[C@H]2C1O ; C=CCOC(=O)N1c2cc(O)c(OC)cc2C(=O)N2CCCC[C@H]2C1O]

A solution of allyl (6aS)-3-(benzyloxy)-6-hydroxy-2-methoxy-12-oxo-6,6a,7,8,9,10-hexahydrobenzo[e]pyrido[1,2-a][1,4]diazepine-5(12H)-carboxylate (37) (861 mg, 1.90 mmol) in anhydrous dichloromethane (30 mL) was cooled to −78° C. and slowly charged with boron trichloride (1 M in toluene) (3.81 mL, 3.80 mmol). The resulting mixture was stirred at the same temperature for 30 min and subsequently quenched by cautious addition of water (5 mL). After diluting with dichloromethane (50 mL) and separating, the organic layer was dried over sodium sulfate, filtered and concentrated in vacuo to give the title compound (407 mg, 59%) as an orange solid, which was used in the subsequent step without further purification.

¹H NMR (400 MHz, CDCl₃) δ 7.18 (s, 1H) 6.75 (s, 1H) 6.30 (br s, 1H) 5.95-5.92 (m, 1H) 5.83-5.77 (m, 1H), 5.18-5.13 (m, 2H), 4.66-4.62 (m, 1H), 4.50-4.47 (m, 1H), 4.37-4.32 (m, 1H), 3.92 (s, 3H), 3.50-3.45 (m, 1H), 3.10-3.03 (m, 1H), 2.08-2.03 (m, 1H), 1.82-1.61 (m, 6H); MS (ES+): m/z=363 (M+H)⁺; LCMS (Method B): t_{R}=2.78 min.

### Example 42: Allyl (6aS)-3-((3-(2-ethoxy-2-oxoethyl)benzyl)oxy)-6-hydroxy-2-methoxy-12-oxo-6,6a,7,8,9,10-hexahydrobenzo[e]pyrido[1,2-a][1,4]-diazepine-5(12H)-carboxylate (39)

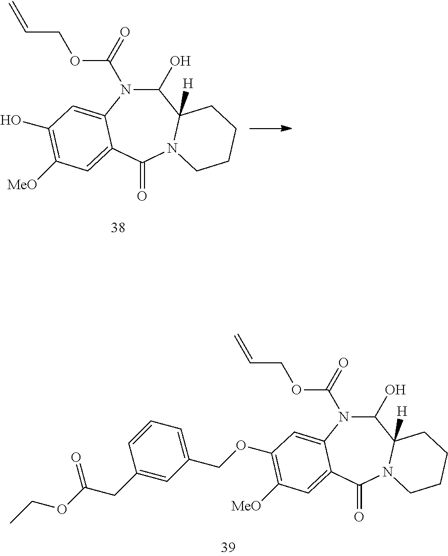 [SMILES: C=CCOC(=O)N1c2cc(O)c(OC)cc2C(=O)N2CCCC[C@H]2C1O ; C=CCOC(=O)N1c2cc(OCc3cccc(CC(=O)OCC)c3)c(OC)cc2C(=O)N2CCCC[C@H]2C1O]

A solution of allyl (6aS)-3,6-dihydroxy-2-methoxy-12-oxo-6,6a,7,8,9,10-hexahydro-benzo[e]pyrido[1,2-a][1,4]diazepine-5(12H)-carboxylate (38) (492 mg, 1.36 mmol) and ethyl 2-(3-(bromomethyl)phenyl)acetate (30) (492 mg, 1.36 mmol) in N,N-dimethyl-formamide (10 mL) was charged with potassium carbonate (282 mg, 2.04 mmol). After stirring at room temperature for 16 h, water (10 mL) was added and the mixture extracted with ethyl acetate (2×15 mL). The combined organic extracts were then washed with an aqueous solution of lithium chloride (1 M, 2×10 mL), dried over sodium sulfate, filtered and concentrated in vacuo. Purification was carried out by column chromatography (silica), eluting with ethyl acetate/hexane (from 0% to 100%) to give the title compound (385 mg, 52%) as a white solid.

¹H NMR (400 MHz, CDCl₃) δ 7.36-7.33 (m, 4H) 7.23-7.22 (m, 1H), 7.20 (s, 1H), 6.70 (br s, 1H), 5.90 (d, J=10.4 Hz, 1H), 5.72-5.70 (m, 1H), 5.14-5.12 (m, 3H), 4.54-4.36 (m, 3H), 4.15 (q, J=70.2 Hz, 2H), 3.94 (s, 3H), 3.62 (s, 2H), 3.50-3.45 (m, 1H), 3.07-3.04 (m, 1H), 2.06-2.02 (m, 1H), 1.81-1.64 (m, 5H), 1.26 (t, J=70.2 Hz, 3H); MS (ES+): m/z=539 (M+H)⁺; LCMS (Method B): t_{R}=3.68 min.

### Example 43: 2-(3-((((6aS)-5-((Allyloxy)carbonyl)-6-hydroxy-2-methoxy-12-oxo-5,6,6a,7,8,9,10,12-octahydrobenzo[e]pyrido[1,2-a][1,4]diazepin-3-yl)-oxy)methyl)phenyl)acetic acid (40)

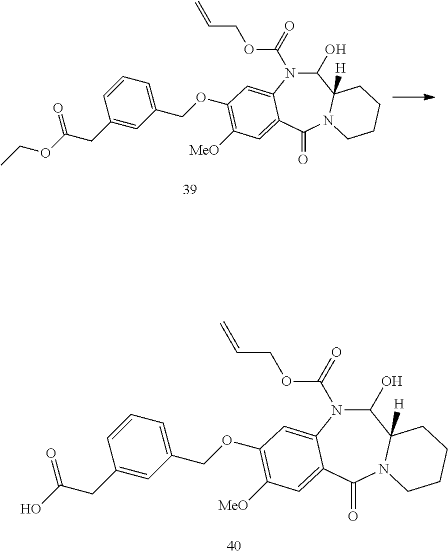 [SMILES: C=CCOC(=O)N1c2cc(OCc3cccc(CC(=O)OCC)c3)c(OC)cc2C(=O)N2CCCC[C@H]2C1O ; C=CCOC(=O)N1c2cc(OCc3cccc(CC(=O)O)c3)c(OC)cc2C(=O)N2CCCC[C@H]2C1O]

A solution of allyl (6aS)-3-((3-(2-ethoxy-2-oxoethyl)benzyl)oxy)-6-hydroxy-2-methoxy-12-oxo-6,6a,7,8,9,10-hexahydrobenzo[e]pyrido[1,2-a][1,4]diazepine-5(12H)-carboxylate (39) (273 mg, 0.51 mmol) in 1,4-dioxane (2 mL) was treated with an aqueous solution of sodium hydroxide (1 M, 2 mL). The reaction mixture was stirred at room temperature for 2 h and then concentrated in vacuo. The resulting residue was dissolved in water (5 mL), acidified with acetic acid to pH=1, and extracted with ethyl acetate (2×5 mL). The combined organics were then washed with brine, dried over sodium sulfate, filtered and concentrated in vacuo to give the title compound (206 mg, 80%) as a white solid, which was used in subsequent steps with no further purification.

¹H NMR (400 MHz, CDCl₃) δ 7.28-7.23 (m, 3H), 7.20-7.14 (m, 2H), 7.10 (s, 1H), 6.62 (br s, 1H), 5.81 (d, J=10.4 Hz, 1H), 5.65-5.57 (m, 1H), 5.05-5.01 (m, 4H), 4.46-4.25 (m, 3H), 3.81 (s, 3H), 3.55 (s, 2H), 3.43-3.37 (m, 1H), 3.01-2.94 (m, 1H), 1.98-1.93 (m, 1H), 1.72-1.51 (m, 5H); MS (ES+): m/z=511 (M+H)⁺; LCMS (Method B): t_{R}=3.22 min.

### Example 44: Allyl (6aS)-3-((3-(2-((S)-1-(chloromethyl)-5-hydroxy-1,2-dihydro-3H-benzo[e]indol-3-yl)-2-oxoethyl)benzyl)oxy)-6-hydroxy-2-methoxy-12-oxo-6,6a,7,8,9,10-hexahydrobenzo[e]pyrido[1,2-a][1,4]-diazepine-5(12H)-carboxylate (41)

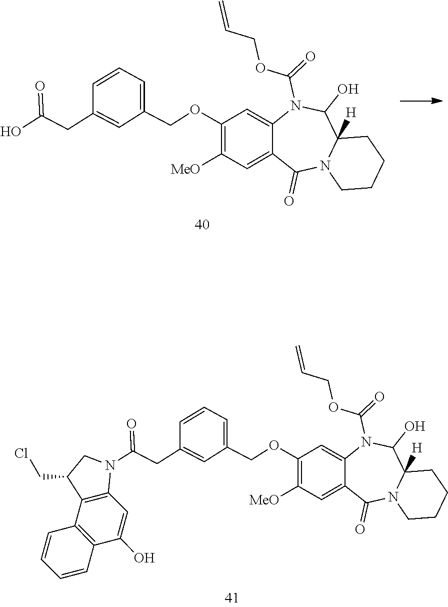 [SMILES: C=CCOC(=O)N1c2cc(OCc3cccc(CC(=O)O)c3)c(OC)cc2C(=O)N2CCCC[C@H]2C1O ; C=CCOC(=O)N1c2cc(OCc3cccc(CC(=O)N4C[C@@H](CCl)c5c4cc(O)c4ccccc54)c3)c(OC)cc2C(=O)N2CCCC[C@H]2C1O]

A solution of 2-(3-((((6aS)-5-((Allyloxy)carbonyl)-6-hydroxy-2-methoxy-12-oxo-5,6,6a,7,8,9,10,12-octahydrobenzo[e]pyrido[1,2-a][1,4]diazepin-3-yl)oxy)methyl)-phenyl)acetic acid (40) (44 mg, 0.087 mmol) in N,N-dimethylacetamide (1.0 mL) was charged with (S)-1-(chloromethyl)-2,3-dihydro-1H-benzo[e]indol-5-ol hydrochloride (2) (20 mg, 0.087 mmol) and N-(3-dimethylaminopropyl)-N′-ethylcarbodiimide hydrochloride (50.0 mg, 0.261 mmol) and stirred at room temperature under argon for 18 h. The reaction mixture was subsequently quenched with a saturated aqueous solution of sodium hydrogen carbonate, and extracted with ethyl acetate (2×50 mL).

The combined organic extracts were then washed with brine (50 mL), dried over magnesium sulfate and concentrated in vacuo. Column chromatography (silica), eluting with ethyl acetate/hexane (from 50% to 100%) afforded the title compound (44 mg, 71%) as a yellow oil.

¹H NMR (400 MHz, CDCl₃) S8.29-8.13 (m, 2H), 7.63 (t, J=90.2 Hz, 1H), 7.54-7.47 (m, 1H), 7.45-7.29 (m, 4H), 7.20 (br s, 1H), 7.15 (d, J=40.2 Hz, 1H), 6.78 (s, 1H), 5.99 (dd, J=14.7, 10.0 Hz, 1H), 5.74-5.61 (m, 1H), 5.41-5.24 (m, 1H), 5.11-4.99 (m, 3H), 4.60-4.50 (m, 1H), 4.49-4.41 (m, 1H), 4.37-4.20 (m, 3H), 4.05-3.93 (m, 2H), 3.91 (s, 3H), 3.38 (t, J=10.7 Hz, 1H), 3.32-3.22 (m, 1H), 3.08-2.93 (m, 3H), 1.98-1.83 (m, 1H), 1.79-1.69 (m, 2H), 1.68-1.60 (m, 3H); ¹³C NMR (100 MHz, CDCl₃) δ 170.3, 169.3, 156.0, 155.0, 149.4, 144.8, 141.2, 141.1, 136.7, 133.8, 131.7, 130.0, 129.4, 129.2, 127.7, 126.4, 126.0, 123.8, 123.6, 122.7, 122.6, 122.1, 117.6, 117.5, 110.3, 100.6, 100.4, 82.3, 71.2, 70.0, 66.7, 56.1, 53.3, 46.6, 46.2, 42.3, 38.9, 31.9, 29.3, 23.0; MS (ES−): m/z=724 (M−1)⁻, MS (ES+): m/z=748 (M+Na)⁺; LCMS (Method C): t_{R}=3.52 min.

### Example 45: (S)-3-((3-(2-((S)-1-(Chloromethyl)-5-hydroxy-1,2-dihydro-3H-benzo[e]indol-3-yl)-2-oxoethyl)benzyl)oxy)-2-methoxy-7,8,9,10-tetrahydrobenzo[e]pyrido[1,2-a][1,4]diazepin-12(6aH)-one (42)

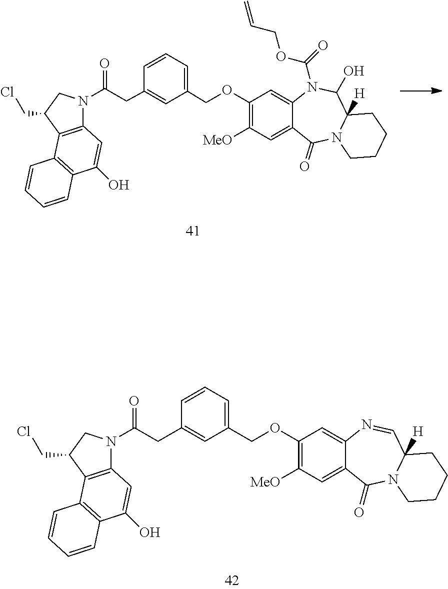 [SMILES: C=CCOC(=O)N1c2cc(OCc3cccc(CC(=O)N4C[C@@H](CCl)c5c4cc(O)c4ccccc54)c3)c(OC)cc2C(=O)N2CCCC[C@H]2C1O ; COc1cc2c(cc1OCc1cccc(CC(=O)N3C[C@@H](CCl)c4c3cc(O)c3ccccc43)c1)N=C[C@@H]1CCCCN1C2=O]

A solution of allyl (6aS)-3-((3-(2-((S)-1-(chloromethyl)-5-hydroxy-1,2-dihydro-3H-benzo [e]indol-3-yl)-2-oxoethyl)benzyl)oxy)-6-hydroxy-2-methoxy-12-oxo-6,6a,7,8,9,10-hexahydrobenzo[e]pyrido[1,2-a][1,4]diazepine-5(12H)-carboxylate (41) (44 mg, 0.061 mmol) in dichloromethane (1 mL) was charged with tetrakis(triphenyl-phosphine)palladium(0) (1 mg) and pyrrolidine (10 μL) and then stirred at room temperature under argon. After approximately 5 min, the resulting mixture was concentrated in vacuo and immediately purified by column chromatography, eluting with acetone/dichloromethane (from 30% to 100%) then with methanol/acetone (100%, isocratic) to give the title compound (19 mg, 50%) as a yellow oil.

¹H NMR (400 MHz, acetone-d₆) δ 9.34 (br s, 1H), 8.21 (d, J=8.6 Hz, 1H), 8.10 (br s, 1H), 7.92 (d, J=5.9 Hz, 1H), 7.80 (d, J=70.8 Hz, 1H), 7.75-7.67 (m, 1H), 7.65-7.59 (m, 1H), 7.57-7.50 (m, 2H), 7.40 (d, J=7.0 Hz, 1H), 7.38-7.34 (m, 2H), 6.84 (br, 1H), 5.26-5.12 (m, 1H), 5.13-5.06 (m, 1H), 4.45-4.39 (m, 2H), 4.37-4.31 (m, 2H), 4.14-4.09 (m, 2H), 4.00-3.93 (m, 2H), 3.82 (s, 3H), 3.73-3.70 (s, 1H), 3.67-3.60 (m, 1H), 2.50 (t, J=7.4 Hz, 1H), 2.32 (dt, J=70.4, 2.0 Hz, 1H), 1.82-1.77 (m, 2H), 1.64-1.55 (br, 2H); MS (ES+): m/z=624 (M+H)⁺; LCMS (Method C): t_{R}=4.02 min, LCMS (Method A): t_{R}=7.60 min.

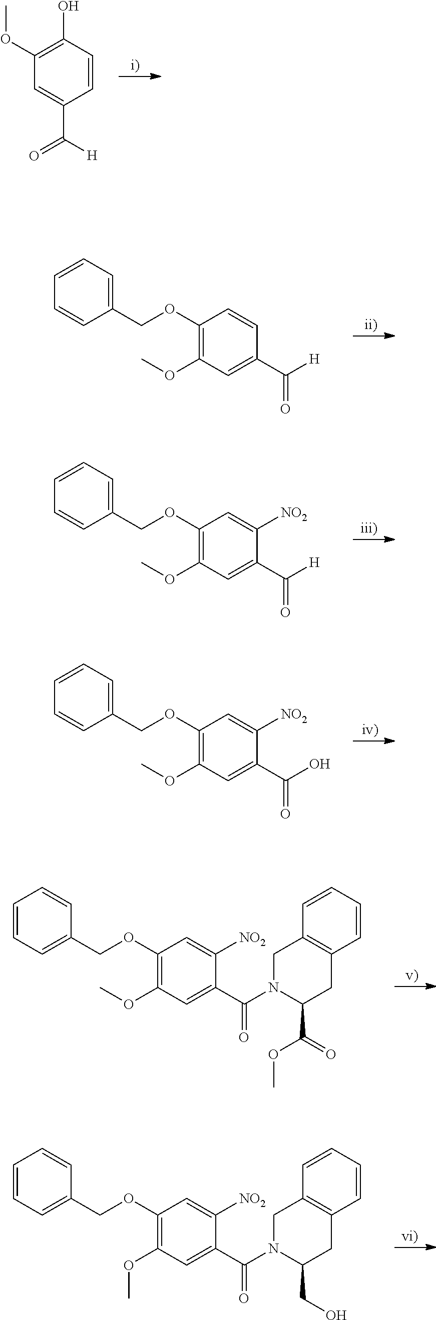 [SMILES: COc1cc(C=O)ccc1O ; COc1cc(C=O)ccc1OCc1ccccc1 ; COc1cc(C=O)c([N+](=O)[O-])cc1OCc1ccccc1 ; COc1cc(C(=O)O)c([N+](=O)[O-])cc1OCc1ccccc1 ; COC(=O)[C@@H]1Cc2ccccc2CN1C(=O)c1cc(OC)c(OCc2ccccc2)cc1[N+](=O)[O-] ; COc1cc(C(=O)N2Cc3ccccc3C[C@H]2CO)c([N+](=O)[O-])cc1OCc1ccccc1]

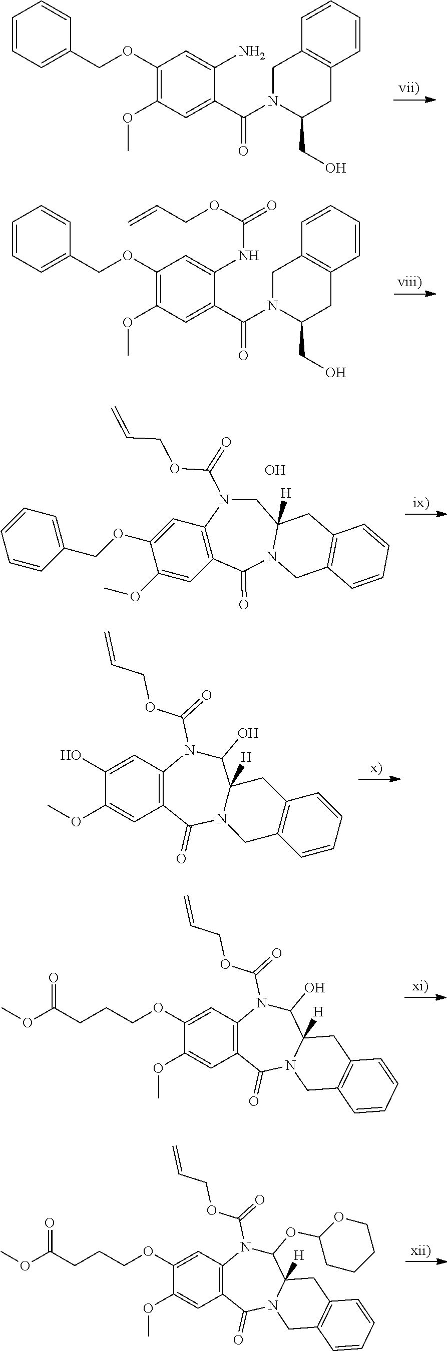

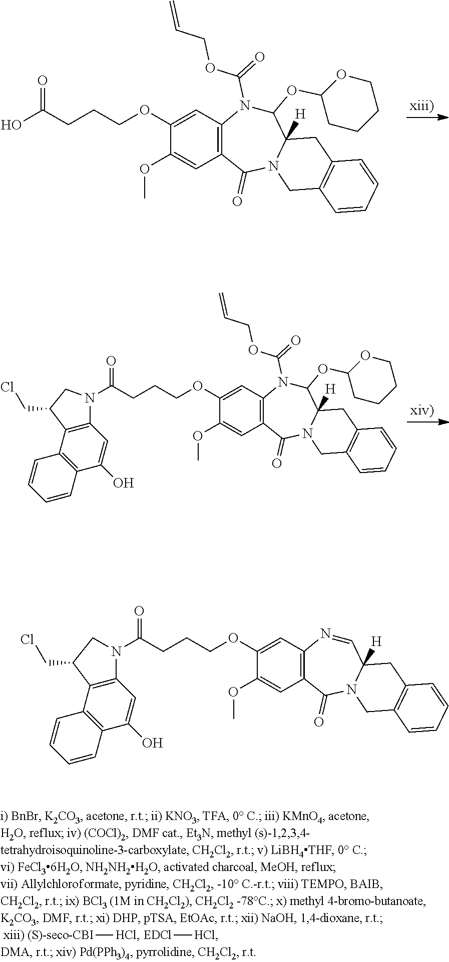

### Example 46: Methyl (S)-2-(4-(benzyloxy)-5-methoxy-2-nitrobenzoyl)-1,2,3,4-tetrahydroisoquinoline-3-carboxylate (43)

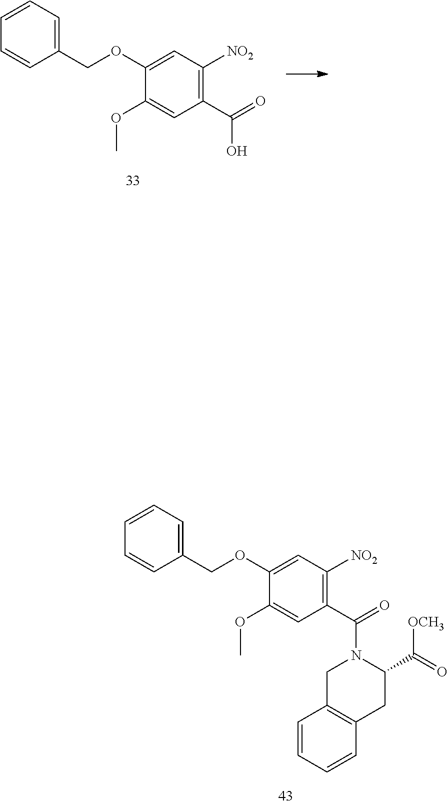 [SMILES: COc1cc(C(=O)O)c([N+](=O)[O-])cc1OCc1ccccc1 ; COC(=O)[C@@H]1Cc2ccccc2CN1C(=O)c1cc(OC)c(OCc2ccccc2)cc1[N+](=O)[O-]]

A mixture of 4-(benzyloxy)-5-methoxy-2-nitrobenzoic acid (33) (2.0 g, 6.6 mmol), oxalyl chloride (1.70 mL, 19.8 mmol) and anhydrous N,N-dimethylformamide (2 drops) in anhydrous dichloromethane (40 mL) was stirred at room temperature for 3 h. Anhydrous toluene (8 mL) was added to the reaction mixture which was then concentrated in vacuo. A solution of the resulting residue in anhydrous dichloro-methane (10 mL) was added dropwise to a solution of methyl (S)-1,2,3,4-tetrahydro-isoquinoline-3-carboxylate (1.65 g, 7.26 mmol) and triethylamine (2.0 mL, 14.5 mmol) in anhydrous dichloromethane (30 mL), at −10° C. The reaction mixture was stirred at room temperature for 2 h and then washed with hydrochloric acid (1 M, 20 mL) and brine (20 mL), dried over sodium sulfate, filtered and concentrated. The resulting residue was purified by column chromatography (silica), eluting with acetone/dichloro-methane (from 0% to 30%), to give the title compound (2.5 g, 79%) as a yellow oil.

¹H NMR (400 MHz, CDCl₃) δ 7.49-7.42 (m, 6H), 7.24-7.19 (m, 5H), 5.25 (s, 2H), 4.64-4.60 (m, 1H), 4.38-4.26 (m, 2H), 3.93 (s, 3H), 3.58 (s, 3H), 3.33-3.23 (m, 2H); ¹³C NMR (100 MHz, CDCl₃) δ 170.8, 170.3, 154.6, 148.4, 135.3, 133.5, 130.5, 130.1, 128.9, 128.8, 128.6, 128.4, 127.7, 127.4, 126.7, 109.3, 109.1, 71.4, 56.8, 52.6, 31.8, 31.0, 30.5; MS (ES+): m/z=477 (M+H)⁺; LCMS (Method B): t_{R}=4.10 min.

### Example 47: (S)-4-(Benzyloxy)-5-methoxy-2-nitrophenyl)(3-(hydroxy-methyl)-3,4-dihydroisoquinolin-2(1H)-yl)methanone (44)

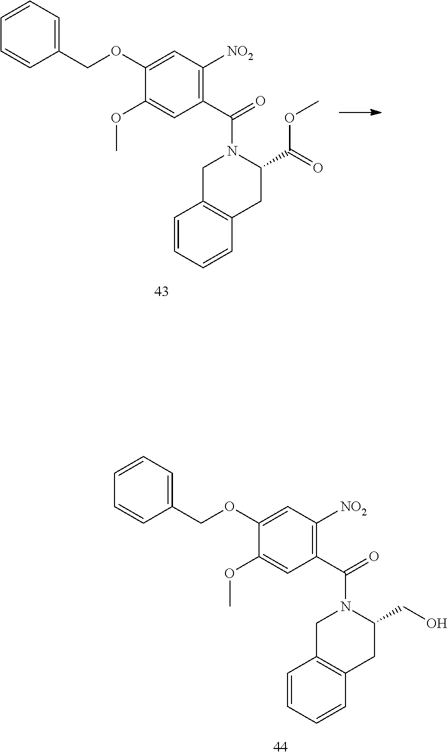 [SMILES: COC(=O)[C@@H]1Cc2ccccc2CN1C(=O)c1cc(OC)c(OCc2ccccc2)cc1[N+](=O)[O-] ; COc1cc(C(=O)N2Cc3ccccc3C[C@H]2CO)c([N+](=O)[O-])cc1OCc1ccccc1]

A solution of methyl (S)-2-(4-(benzyloxy)-5-methoxy-2-nitrobenzoyl)-1,2,3,4-tetra-hydroisoquinoline-3-carboxylate (43) (2.4 g, 5.0 mmol) in anhydrous tetrahydrofuran (48 mL) was charged with a solution of lithium borohydride (2 M in tetrahydrofuran, 3.8 mL, 8.7 mmol) at 0° C. The reaction mixture was stirred at room temperature for 3 hours. Water (150 mL) was added dropwise at 0° C. and the reaction mixture was then extracted with ethyl acetate (2×100 mL). The combined organic extracts were then concentrated in vacuo. The resulting residue was purified by column chromatography (silica), eluting with acetone/dichloromethane (from 0% to 30%), to give the title compound (2.2 g, 97%) as creamy oil.

¹H NMR (400 MHz, CDCl₃) δ 7.42-7.39 (m, 4H), 7.36-7.34 (m, 5H), 7.30 (s, 1H), 7.29 (s, 1H), 5.17 (s, 2H), 4.62 (s, 1H), 4.36-4.25 (m, 1H), 4.23-4.16 (m, 2H), 3.87 (s, 3H), 3.70-3.63 (m, 1H), 3.58-3.50 (m, 1H), 3.05-2.97 (m, 2H); ¹³C NMR (100 MHz, CDCl₃) δ 168.2, 150.2, 148.3, 133.7, 128.9, 128.9, 128.8, 128.6, 127.7, 127.6, 127.5, 127.0, 126.5, 114.4, 110.6, 108.9, 103.9, 91.6, 71.4, 65.4, 54.4, 33.3; MS (ES+): m/z=449 (M+H)⁺; LCMS (Method B): t_{R}=3.78 min.

### Example 48: (S)-(2-Amino-4-(benzyloxy)-5-methoxyphenyl)(3-(hydroxy-methyl)-3,4-dihydroisoquinolin-2(1H)-yl)methanone (45)

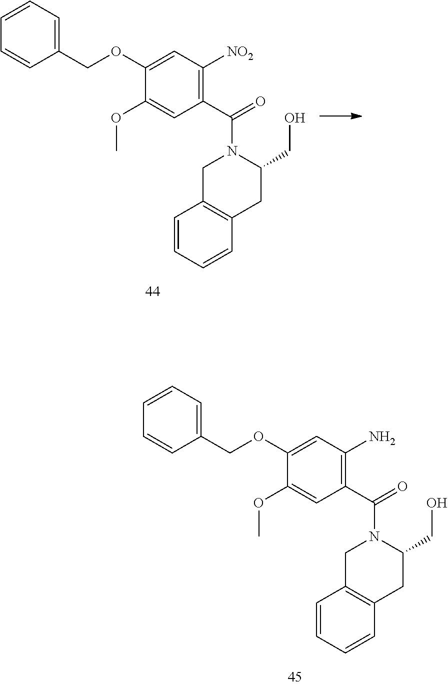 [SMILES: COc1cc(C(=O)N2Cc3ccccc3C[C@H]2CO)c([N+](=O)[O-])cc1OCc1ccccc1 ; COc1cc(C(=O)N2Cc3ccccc3C[C@H]2CO)c(N)cc1OCc1ccccc1]

A solution of (S)-(4-(benzyloxy)-5-methoxy-2-nitrophenyl)(3-(hydroxymethyl)-3,4-dihydroisoquinolin-2(1H)-yl)methanone (44) (2.20 g, 4.90 mmol) in tetrahydrofuran (50 mL) and methanol (50 mL) was charged with iron (III) chloride hexahydrate (0.80 g, 2.90 mmol), activated charcoal (2.60 g, 221 mmol) and hydrazine (2.90 mL, 58.9 mmol). The reaction mixture was then stirred at reflux (85° C.) for 16 h. The mixture was subsequently allowed to cool to room temperature and filtered through a plug of celite. The filter cake was washed with ethyl acetate and methanol and then concentrated in vacuo to give the title compound (1.7 g, 83%) as brown solid.

¹H NMR (400 MHz, MeOD) δ 7.48 (s, 1H), 7.46 (s, 1H), 7.41-7.33 (m, 4H), 7.20-7.18 (m, 3H), 6.84 (s, 1H), 6.56 (s, 1H), 5.11 (s, 2H), 4.61 (s, 1H), 4.54-4.40 (m, 1H), 3.77 (s, 3H), 3.62-3.54 (m, 2H), 3.19 (dd, J=16.2, 5.9 Hz, 2H), 2.92-2.80 (m, 2H); ¹³C NMR (100 MHz, MeOD) δ 169.1, 149.8, 141.0, 135.5, 130.7, 129.0, 128.7, 128.6, 128.5, 128.4, 128.2, 127.4, 127.0, 126.7, 110.1, 109.1, 71.0, 68.7, 64.8, 56.4, 50.3, 27.9; MS (ES+): m/z=419 (M+H)⁺; LCMS (Method B): t_{R}=3.50 min.

### Example 49: Allyl (S)-(5-(benzyloxy)-2-(3-(hydroxymethyl)-1,2,3,4-tetra-hydroisoquinoline-2-carbonyl)-4-methoxyphenyl)carbamate (46)

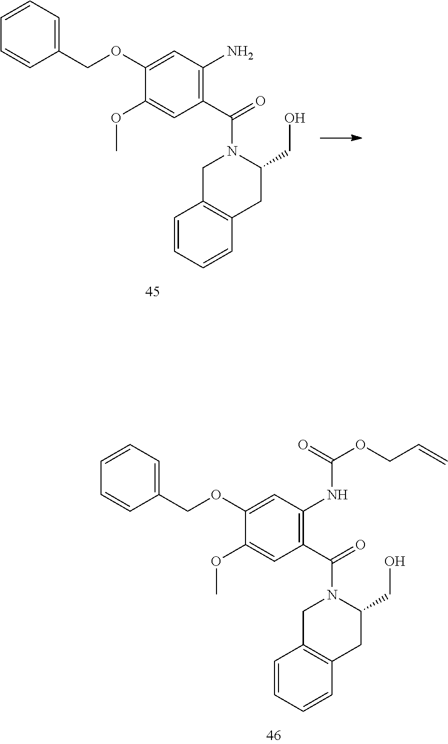 [SMILES: COc1cc(C(=O)N2Cc3ccccc3C[C@H]2CO)c(N)cc1OCc1ccccc1 ; C=CCOC(=O)Nc1cc(OCc2ccccc2)c(OC)cc1C(=O)N1Cc2ccccc2C[C@H]1CO]

A solution of (S)-(2-amino-4-(benzyloxy)-5-methoxyphenyl)(3-(hydroxymethyl)-3,4-dihydroisoquinolin-2(1H)-yl)methanone (45) (1.50 g, 3.6 mmol) and anhydrous pyridine (696 μL, 8.97 mmol) in anhydrous dichloromethane (50 mL) at −10° C. was slowly charged with a solution of allylchloroformate (343 μL, 3.23 mmol) in anhydrous dichloromethane (30 mL). The reaction mixture was stirred at room temperature for 30 min and then sequentially washed with a saturated aqueous solution of copper (II) sulfate (50 mL), water (50 mL) and a saturated aqueous solution of sodium hydrogen carbonate (50 mL). The organic layer was dried over sodium sulfate, filtered and concentrated in vacuo. The resulting residue was purified by column chromatography (silica), eluting with acetone/dichloromethane (from 0% to 20%), to give the title compound (1.47 g, 81%) as an off-white solid.

¹H NMR (400 MHz, MeOD) δ 8.14 (s, 1H), 7.81 (s, 1H), 7.51 (s, 1H), 7.49 (s, 1H), 7.42-7.32 (m, 4H), 7.23-7.17 (m, 3H), 6.82 (s, 1H), 5.97-5.87 (m, 1H), 5.33 (dq, J=17.2, 1.5 Hz, 1H), 5.22 (dq, J=10.6, 1.3 Hz, 1H), 5.19 (s, 2H), 4.68-4.64 (m, 1H), 4.61 (dd, J=5.5, 1.3 Hz, 2H), 4.44 (br. s, 2H), 3.82 (s, 3H), 3.70-3.64 (m, 1H), 3.21-3.15 (m, 1H), 2.74 (br. s, 1H); ¹³C NMR (100 MHz, CDCl₃) δ 169.4, 152.9, 148.7, 144.1, 140.1, 135.3, 131.4, 130.5, 129.1, 128.1, 127.5, 127.0, 126.7, 125.9, 125.5, 117.9, 116.8, 109.6, 105.7, 69.7, 67.4, 66.0, 64.7, 55.3, 53.8, 26.8; MS (ES+): m/z=503 (M+H)⁺; LCMS (Method B): t_{R}=3.95 min.

### Example 50: Allyl (6aS)-3-(benzyloxy)-6-hydroxy-2-methoxy-14-oxo-6,6a,7,12-tetrahydrobenzo[5,6][1,4]diazepino[1,2-b]isoquinoline-5(14H)-carboxylate (47)

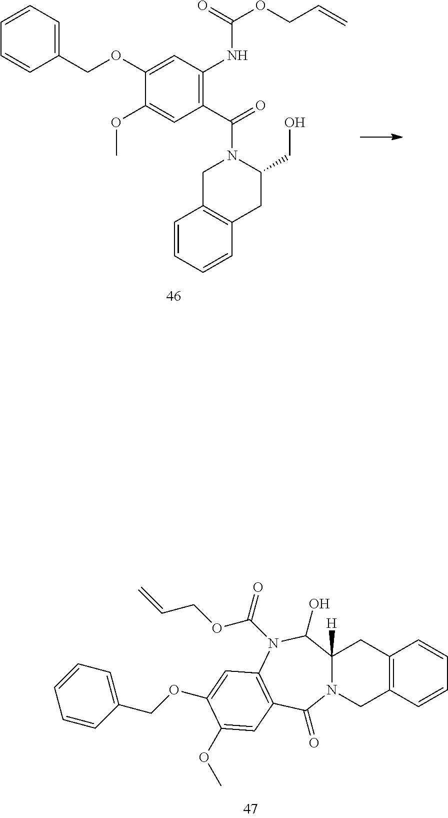 [SMILES: C=CCOC(=O)Nc1cc(OCc2ccccc2)c(OC)cc1C(=O)N1Cc2ccccc2C[C@H]1CO ; C=CCOC(=O)N1c2cc(OCc3ccccc3)c(OC)cc2C(=O)N2Cc3ccccc3C[C@H]2C1O]

A solution of allyl (S)-(5-(benzyloxy)-2-(3-(hydroxymethyl)-1,2,3,4-tetrahydro-isoquinoline-2-carbonyl)-4-methoxyphenyl)carbamate (46) (1.4 g, 2.78 mmol) in dichloromethane (80 mL) was charged with 2,2,6,6-tetramethyl-1-piperidinyloxy (44 mg, 0.28 mmol) and (diacetoxyiodo)benzene (1.0 g, 3.33 mmol). The reaction mixture was stirred at room temperature for 16 h and was then sequentially washed with a saturated aqueous solution of sodium metabisulfite (40 mL), a saturated aqueous solution of sodium hydrogen carbonate (40 mL), water (30 mL) and brine (30 mL). The organic layer was then dried over sodium sulfate, filtered and concentrated. The resulting residue was purified by column chromatography (silica), eluting with acetone/dichloromethane (from 0% to 20%), to give the title compound (1.2 g, 86%) as an off-white solid.

¹H NMR (400 MHz, CDCl₃) δ 7.44-7.31 (m, 6H), 7.28-7.26 (m, 5H), 6.72 (s, 1H), 5.70-5.61 (m, 1H), 5.31 (d, J=9.8 Hz, 1H), 5.20-5.17 (m, 1H), 5.11-5.07 (m, 3H), 4.83 (d, J=15.6 Hz, 1H), 4.58 (d, J=15.6 Hz, 1H), 4.48-4.34 (m, 2H), 3.94 (s, 3H), 3.74-3.69 (m, 1H), 3.17-3.05 (m, 2H); ¹³C NMR (100 MHz, CDCl₃) δ 169.0, 149.0, 136.2, 134.3, 133.7, 131.8, 126.7, 128.2, 127.9, 127.8, 127.3, 126.7, 118.1, 114.0, 111.2, 84.8, 71.0, 66.7, 56.2, 53.5, 50.8, 44.3, 30.2; MS (ES+): m/z=501 (M+H)⁺; LCMS (Method B): t_{R}=3.80 min.

### Example 51: Allyl (6aS)-3,6-dihydroxy-2-methoxy-14-oxo-6,6a,7,12-tetra-hydrobenzo[5,6][1,4]diazepino[1,2-b]isoquinoline-5(14H)-carboxylate (48)

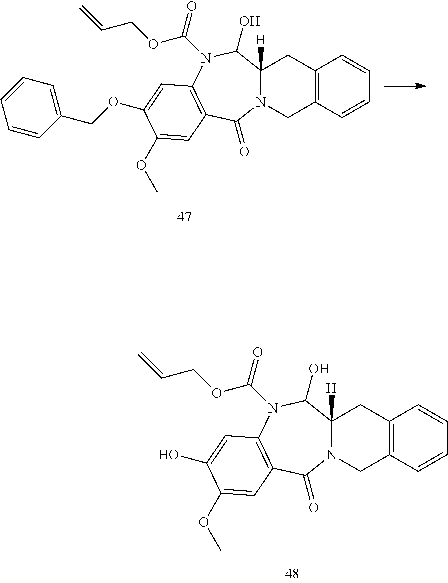 [SMILES: C=CCOC(=O)N1c2cc(OCc3ccccc3)c(OC)cc2C(=O)N2Cc3ccccc3C[C@H]2C1O ; C=CCOC(=O)N1c2cc(O)c(OC)cc2C(=O)N2Cc3ccccc3C[C@H]2C1O]

A solution of allyl (6aS)-3-(benzyloxy)-6-hydroxy-2-methoxy-14-oxo-6,6a,7,12-tetra-hydrobenzo[5,6][1,4]diazepino[1,2-b]isoquinoline-5(14H)-carboxylate (47) (1.10 g, 2.20 mmol) in anhydrous dichloromethane (20 mL) was charged with a solution of boron trichloride (1 M in hexane, 4.4 mL, 4.4 mmol) at −78° C. The resulting mixture was stirred for 5 h at −78° C. and then quenched via dropwise addition of water (5 mL).

An aqueous acetic acid solution (50 mL) was added to adjust to pH=3, and the resulting mixture was then extracted with ethyl acetate (2×60 mL). The combined organic extracts were then concentrated in vacuo. The resulting residue was purified by column chromatography (silica), eluting with acetone/dichloromethane (from 0% to 30%), to give the title compound (860 mg, 95%) as a pale yellow solid.

¹H NMR (400 MHz, CDCl₃) δ 7.29-7.26 (m, 6H), 6.76 (s, 1H), 6.02 (s, 1H), 5.84-5.75 (m, 1H), 5.34-5.31 (m, 1H), 5.17-5.13 (m, 2H), 4.83 (d, J=15.6 Hz, 1H), 4.64-4.56 (m, 2H), 4.46-4.43 (m, 1H), 3.95 (s, 3H), 3.75-3.70 (m, 1H), 3.19-3.06 (m, 2H); ¹³C NMR (100 MHz, CDCl₃) δ 169.0, 159.4, 148.0, 146.0, 134.3, 133.7, 131.8, 127.9, 127.8, 127.3, 126.7, 118.1, 115.3, 110.6, 84.8, 66.8, 56.3, 44.3, 31.0, 30.2; MS (ES+): m/z=411 (M+H)⁺; LCMS (Method B): t_{R}=3.15 min.

### Example 52: Allyl (6aS)-6-hydroxy-2-methoxy-3-(4-methoxy-4-oxobutoxy)-14-oxo-6,6a,7,12-tetrahydrobenzo[5,6][1,4]diazepino[1,2-b]isoquinoline-5(14H-carboxylate (49)

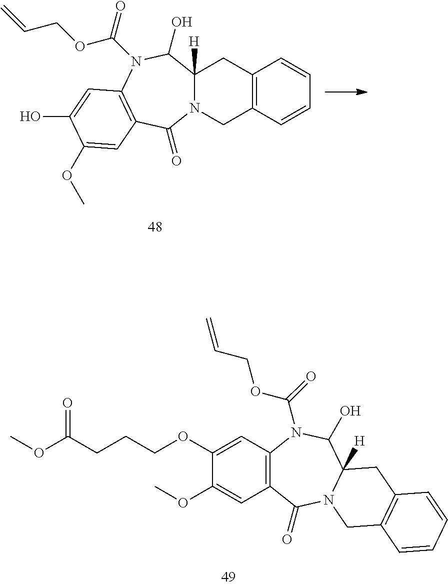 [SMILES: C=CCOC(=O)N1c2cc(O)c(OC)cc2C(=O)N2Cc3ccccc3C[C@H]2C1O ; C=CCOC(=O)N1c2cc(OCCCC(=O)OC)c(OC)cc2C(=O)N2Cc3ccccc3C[C@H]2C1O]

A solution of allyl (6aS)-3,6-dihydroxy-2-methoxy-14-oxo-6,6a,7,12-tetrahydrobenzo-[5,6][1,4]diazepino[1,2-b]isoquinoline-5(14H)-carboxylate (48) (300 mg, 0.73 mmol) in N,N-dimethylformamide (3 mL) was charged with methyl 4-bromobutanoate (166 μL, 1.31 mmol) and potassium carbonate (151 mg, 1.10 mmol) and stirred at room temperature under an inert atmosphere of argon for 20 h. The reaction mixture was diluted with water (30 mL) and extracted with ethyl acetate (3×20 mL). The combined organic extracts were then washed with brine (20 mL), dried over magnesium sulfate, filtered and concentrated in vacuo to give the title compound (368 mg, 99%) as a yellow oil, which was carried through to the subsequent step without further purification.

¹H NMR (400 MHz, CDCl₃) δ 7.30 (s, 5H), 6.75 (br. s, 1H), 5.86-5.74 (m, 1H), 5.38 (d, J=9.8 Hz, 1H), 5.13 (d, J=11.3 Hz, 2H) 4.83 (d, J=15.6 Hz, 1H), 4.43 (br. s, 1H), 4.08 (q, J=5.9 Hz, 2H), 3.94-3.91 (m, 3H), 3.71 (s, 3H), 3.50 (t, J=6.4 Hz, 2H) 3.07-3.20 (m, 2H), 2.56-2.59 (m, 2H), 2.25-2.17 (m, 4H); ¹³C NMR (100 MHz, CDCl₃) δ 173.3, 172.9, 169.0, 148.7, 134.3, 133.8, 131.9, 127.7, 127.7, 127.1, 126.6, 124.9, 117.7, 113.6, 111.1, 84.7, 67.9, 66.5, 56.0, 55.8, 51.6, 51.6, 44.2, 32.6, 30.3, 27.7, 24.2; MS (ES+): m/z=511 (M+H)⁺, MS (ES−): m/z=509 (M−1)⁻; LCMS (Method B): t_{R}=3.63 min, LCMS (Method A): t_{R}=6.97 min.

### Example 53: Allyl (6aS)-2-methoxy-3-(4-methoxy-4-oxobutoxy)-14-oxo-6-((tetrahydro-2H-pyran-2-yl)oxy)-6,6a,7,12-tetrahydrobenzo[5,6][1,4]-diazepino[1,2-b]isoquinoline-5(14H)-carboxylate (50)

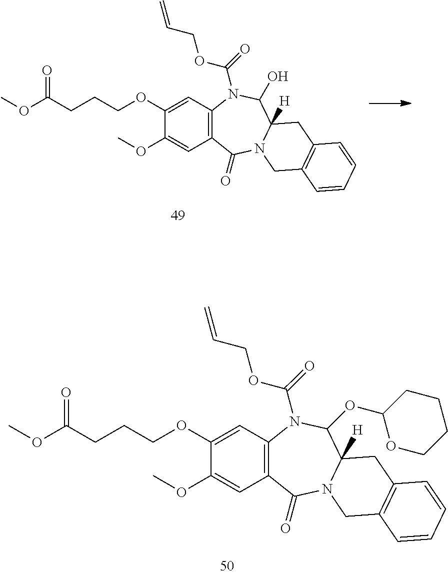 [SMILES: C=CCOC(=O)N1c2cc(OCCCC(=O)OC)c(OC)cc2C(=O)N2Cc3ccccc3C[C@H]2C1O ; C=CCOC(=O)N1c2cc(OCCCC(=O)OC)c(OC)cc2C(=O)N2Cc3ccccc3C[C@H]2C1OC1CCCCO1]

A solution of allyl (6aS)-6-hydroxy-2-methoxy-3-(4-methoxy-4-oxobutoxy)-14-oxo-6,6a,7,12-tetrahydrobenzo[5,6][1,4]diazepino[1,2-b]isoquinoline-5(14H)-carboxylate (49) (367 mg, 0.72 mmol) in ethyl acetate (2 mL) was charged with p-toluenesulfonic acid monohydrate (3.7 mg, 1% w/w) and 3,4-dihydro-2H-pyran (657 μL, 7.20 mmol). The resulting mixture was stirred at room temperature for 20 h, then diluted with ethyl acetate (15 mL) and subsequently washed with a saturated aqueous solution of sodium hydrogen carbonate (10 mL), water (15 mL) and brine (15 mL), dried over magnesium sulfate, filtered and concentrated in vacuo. Column chromatography (silica gel), eluting ethyl acetate/petroleum ether (50%, isocratic) afforded the title compound (390 mg, 91%) as a light-yellow gel.

¹H NMR (400 MHz, CDCl₃) δ 7.73-7.70 (m, 1H), 7.56-7.52 (m, 1H), 7.30-7.28 (m, 1H), 7.23 (d, J=8.2 Hz, 1H), 6.86 (s, 1H), 6.60 (s, 1H), 5.81-5.63 (m, 1H), 5.46 (d, J=9.4 Hz, 1H), 5.11-5.03 (m, 2H), 4.79 (d, J=15.6 Hz, 1H), 4.74-4.48 (m, 2H), 4.48-4.31 (m, 1H), 4.28-4.18 (m, 2H), 4.06 (q, J=6.0 Hz, 2H), 3.91 (s, 3H), 3.69 (s, 3H), 3.63-3.51, (m, 2H), 3.25-3.17 (m, 1H), 3.12-3.05 (m, 1H), 2.62-2.47 (m, 2H), 2.26-2.10 (m, 2H), 1.90-1.65 (m, 3H), 1.58 (d, J=10.9 Hz, 3H); ¹³C NMR (100 MHz, CDCl₃) δ 173.4, 169.2, 167.7, 149.2, 134.7, 132.4, 130.8, 128.8, 126.5, 117.2, 117.1, 114.5, 113.9, 111.2, 110.8, 99.9, 90.2, 67.7, 68.1, 66.3, 63.6, 56.1, 51.6, 44.2, 31.1, 30.4, 28.9, 25.2, 23.7, 23.0, 20.1, 10.9; MS (ES+): m/z=595 (M+H)⁺; LCMS (Method B): t_{R}=4.35 min, LCMS (Method A): t_{R}=8.27 min.

### Example 54: 4-(((6aS)-5-((Allyloxy)carbonyl)-2-methoxy-14-oxo-6-((tetra-hydro-2H-pyran-2-yl)oxy)-5,6,6a,7,12,14-hexahydrobenzo[5,6][1,4]-diazepino[1,2-b]isoquinolin-3-yl)oxy)butanoic acid (51)

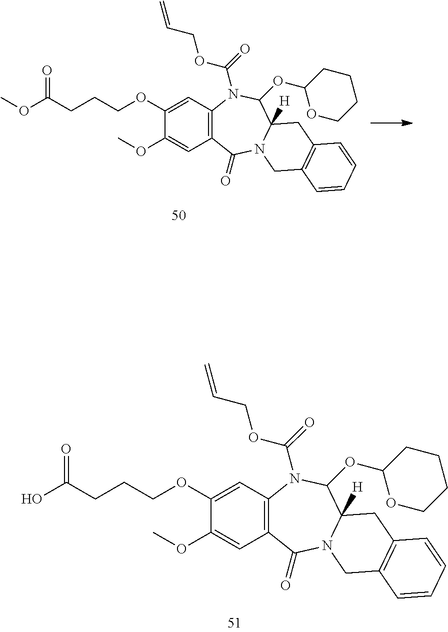 [SMILES: C=CCOC(=O)N1c2cc(OCCCC(=O)OC)c(OC)cc2C(=O)N2Cc3ccccc3C[C@H]2C1OC1CCCCO1 ; C=CCOC(=O)N1c2cc(OCCCC(=O)O)c(OC)cc2C(=O)N2Cc3ccccc3C[C@H]2C1OC1CCCCO1]

A solution of allyl (6aS)-2-methoxy-3-(4-methoxy-4-oxobutoxy)-14-oxo-6-((tetrahydro-2H-pyran-2-yl)oxy)-6,6a,7,12-tetrahydrobenzo[5,6][1,4]diazepino[1,2-b]isoquinoline-5(14H)-carboxylate (50) (332 mg, 0.55 mmol) in 1,4-dioxane (1 mL) was charged with an aqueous solution of sodium hydroxide (1 M, 1.20 mL, 1.2 mmol) and stirred at room temperature for 15 h. The reaction mixture was then concentrated in vacuo, whereupon water (10 mL) was added and the suspension was acidified to pH=1 with an aqueous solution of citric acid (1 M). The aqueous layer was then extracted with ethyl acetate (3×15 mL) and the combined organic extracts were then washed with brine (15 mL) and concentrated in vacuo to give the title compound (278 mg, 87%) as a white solid.

¹H NMR (400 MHz, CDCl₃) δ 7.78-7.69 (m, 1H), 7.60-7.53 (m, 1H), 7.32-7.30 (m, 1H), 7.30 (br. s, 1H), 6.89 (s, 1H), 6.61 (br. s, 1H), 5.82-5.62 (m, 1H), 5.47 (d, J=9.8 Hz, 1H), 5.13-5.03 (m, 2H), 4.82 (d, J=16.0 Hz, 1H), 4.73-4.55 (m, 2H), 4.30-4.20 (m, 2H), 4.18-4.06 (m, 2H), 3.99 (dd, J=10.7, 5.3 Hz, 1H), 3.93 (s, 3H), 3.80-3.68 (m, 1H), 3.60 (br. s, 1H), 3.21 (d, J=30.1 Hz, 1H), 3.15-3.08 (m, 1H), 2.61 (q, J=7.3 Hz, 2H), 2.18 (quin, J=6.6 Hz, 2H), 1.89-1.67 (m, 3H), 1.65-1.53 (m, 3H); ¹³C NMR (100 MHz, CDCl₃) δ 177.8, 169.4, 169.4, 167.8, 149.2, 149.0, 134.6, 132.4, 130.9, 128.8, 127.7, 127.5, 126.8, 126.5, 117.2, 68.1, 67.6, 66.3, 63.3, 56.1, 44.2, 38.7, 31.1, 30.3, 28.9, 25.3, 23.7, 23.0, 20.0, 14.0, 10.9; MS (ES+): m/z=581 (M+H)⁺, MS (ES−): m/z=579 (M−1)⁻; LCMS (Method B): t_{R}=3.93 min, LCMS (Method A): t_{R}=7.53 min.

### Example 55: Allyl (6aS)-3-(4-((S)-1-(chloromethyl)-5-hydroxy-1,2-dihydro-3H-benzo[e]indol-1-yl)-4-oxobutoxy)-2-methoxy-14-oxo-6-((tetrahydro-2H-pyran-2-yl)oxy)-6,6a,7,12-tetrahydrobenzo[5,6][1,4]diazepino[1,2-b]isoquinoline-5(14H-carboxylate (52)

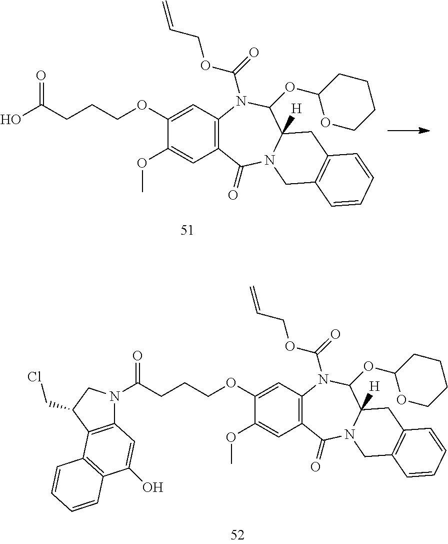 [SMILES: C=CCOC(=O)N1c2cc(OCCCC(=O)O)c(OC)cc2C(=O)N2Cc3ccccc3C[C@H]2C1OC1CCCCO1 ; C=CCOC(=O)N1c2cc(OCCCC(=O)N3C[C@@H](CCl)c4c3cc(O)c3ccccc43)c(OC)cc2C(=O)N2Cc3ccccc3C[C@H]2C1OC1CCCCO1]

A solution of 4-(((6aS)-5-((allyloxy)carbonyl)-2-methoxy-14-oxo-6-((tetrahydro-2H-pyran-2-yl)oxy)-5,6,6a,7,12,14-hexahydrobenzo[5,6][1,4]diazepino[1,2-b]isoquinolin-3-yl)oxy)butanoic acid (51) (109 mg, 0.188 mmol) in N,N-dimethylacetamide (4 mL) was charged with (S)-1-(chloromethyl)-2,3-dihydro-1H-benzo[e]indol-5-ol hydrochloride (2) (38.0 mg, 0.188 mmol) and N-(3-dimethylaminopropyl)-N′-ethylcarbodiimide hydrochloride (108 mg, 0.56 mmol) and stirred at room temperature under argon for 18 h. The reaction mixture was subsequently quenched with a saturated aqueous solution of sodium hydrogen carbonate, then extracted with ethyl acetate (3×60 mL). The combined organic extracts were then washed with brine (80 mL), dried over magnesium sulfate and concentrated in vacuo. Column chromatography (silica), eluting with ethyl acetate/hexane (from 25% to 100%) afforded the title compound (62 mg, 46%) as a grey oil.

¹H NMR (400 MHz, CDCl₃) δ 8.27 (d, J=9.0 Hz, 1H), 7.64 (d, J=8.2, 1H), 7.52-7.48 (m, 1H), 7.38-7.34 (m, 1H), 7.27-7.18 (m, 6H), 6.94 (s, 1H), 5.70-5.63 (m, 1H), 5.04-4.95 (m, 2H), 4.79-4.48 (m, 2H), 4.35-4.19 (m, 4H), 4.03-3.88 (m, 6H), 3.68 (br. s, 1H), 3.59-3.37 (m, 2H), 3.16-2.72 (m, 4H), 2.39-2.28 (m, 2H), 1.70-1.41 (m, 8H), 1.33-1.28 (m, 2H); MS (ES−): m/z=794 (M−1)⁻; LCMS (Method C): t_{R}=4.15 min.

### Example 56: (S)-3-(4-((S)-1-(Chloromethyl)-5-hydroxy-1,2-dihydro-3H-benzo[e]indol-3-yl)-4-oxobutoxy)-2-methoxy-7,12-dihydrobenzo[5,6][1,4]-diazepino[1,2-b]isoquinolin-14(6aH)-one (53)

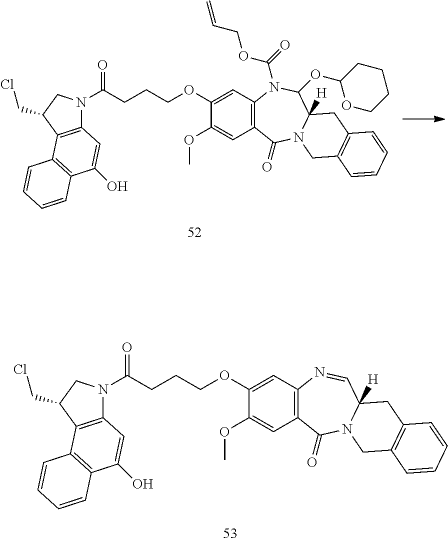 [SMILES: C=CCOC(=O)N1c2cc(OCCCC(=O)N3C[C@@H](CCl)c4c3cc(O)c3ccccc43)c(OC)cc2C(=O)N2Cc3ccccc3C[C@H]2C1OC1CCCCO1 ; COc1cc2c(cc1OCCCC(=O)N1C[C@@H](CCl)c3c1cc(O)c1ccccc31)N=C[C@@H]1Cc3ccccc3CN1C2=O]

A solution of allyl (6aS)-3-(4-((S)-1-(chloromethyl)-5-hydroxy-1,2-dihydro-3H-benzo-[e]indol-3-yl)-4-oxobutoxy)-2-methoxy-14-oxo-6-((tetrahydro-2H-pyran-2-yl)oxy)-6,6a,7,12-tetrahydrobenzo[5,6][1,4]diazepino[1,2-b]isoquinoline-5(14H)-carboxylate (52) (32 mg, 0.041 mmol) in dichloromethane (4 mL) was charged with tetrakis-(triphenylphosphine)palladium(0) (4 mg) and pyrrolidine (10 μL) and then stirred at room temperature under argon. After approximately 10 min, the resulting mixture was concentrated in vacuo and immediately purified by column chromatography, eluting with methanol/ethyl acetate (from 0% to 5%), to give the title compound (3 mg, 12%) as a yellow oil.

¹H NMR (400 MHz, acetone-d₆) δ 9.38 (br s, 1H), 8.21 (d, J=8.2 Hz, 1H), 8.14 (s, 1H), 7.80 (d, J=8.6 Hz, 1H), 7.72 (d, J=70.8 Hz, 1H), 7.69 (d, J=7.4 Hz, 1H), 7.61 (d, J=6.6 Hz, 1H), 7.58-7.48 (m, 2H), 7.44 (s, 1H), 7.38-7.28 (m, 2H), 6.84 (s, 1H), 4.89 (d, J=15.2 Hz, 1H), 4.56 (dd, J=15.2, 2.5 Hz, 1H), 4.40-4.31 (m, 2H), 4.23 (dt, J=6.3, 3.3 Hz, 2H), 4.03-3.98 (m, 1H), 3.95-3.89 (m, 1H), 3.87 (s, 3H), 3.75-3.67 (m, 1H), 3.34-3.29 (m, 2H), 2.79-2.69 (m, 1H), 2.26-2.21 (m, 2H), 1.62-1.53 (m, 1H), 1.40-1.35 (m, 1H); ¹³C NMR (100 MHz, acetone-d₆) δ 170.0, 167.4, 159.0, 155.6, 151.1, 146.8, 145.6, 134.4, 134.0, 131.9, 130.4, 128.6, 128.5, 127.9, 127.7, 127.0, 126.2, 123.3, 122.7, 122.4, 114.4, 112.1, 110.3, 110.0, 104.5, 67.8, 55.4, 52.9, 49.4, 43.2, 31.6, 30.2, 26.0; MS (ES+): m/z=610 (M+H)⁺; LCMS (Method C): t_{R}=3.55 min.

Reaction Scheme for Preparing Compound (55)

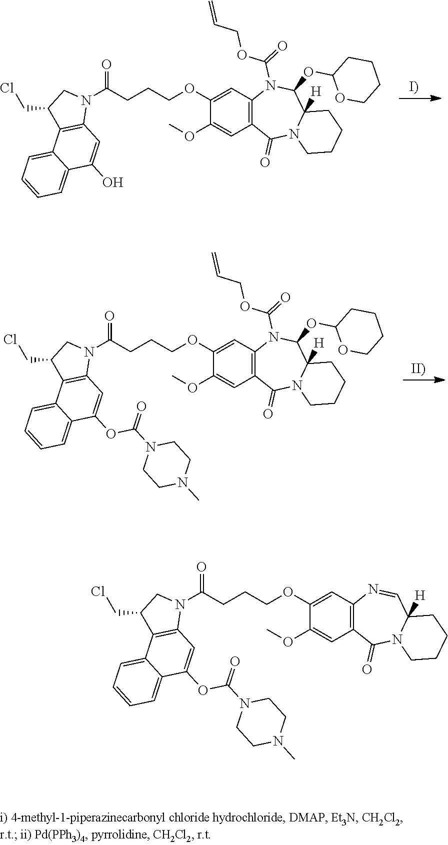

### Example 57: Allyl (6S,6aS)-3-(4-((S)-1-(chloromethyl)-5-((4-methyl-piperazine-1-carbonyl)oxy)-1,2-dihydro-3H-benzo[e]indol-3-yl)-4-oxobutoxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (54)

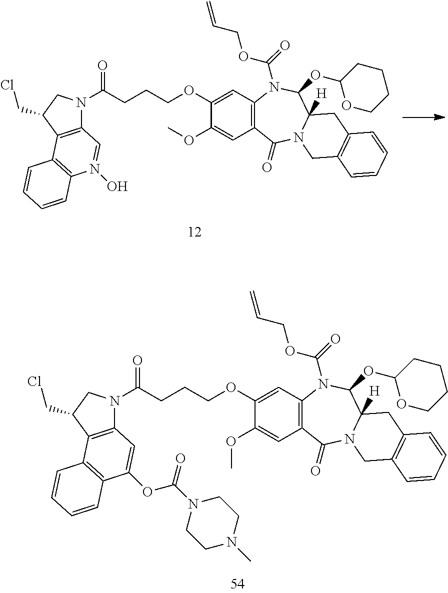

A solution of allyl (6S,6aS)-3-(4-((S)-1-(chloromethyl)-5-hydroxy-1,2-dihydro-3H-benzo[e]indol-3-yl)-4-oxobutoxy)-2-methoxy-12-oxo-6-((tetrahydro-2H-pyran-2-yl)-oxy)-6,6a,7,8,9,10-hexahydrobenzo[e]pyrido[1,2-a][1,4]diazepine-5(12H)-carboxylate (17) (177 mg, 0.237 mmol) in dichloromethane (3 mL) was charged with 4-methyl-1-piperazinecarbonyl chloride hydrochloride (141 mg, 0.710 mmol), 4-(dimethylamino)-pyridine (32 mg, 0.26 mmol) and triethylamine (115 μL, 0.83 mmol) and stirred at room temperature for 18 h. The reaction mixture was subsequently washed with water (2×10 mL), dried over magnesium sulfate and concentrated in vacuo, to give the title compound (95 mg, 46%) as a yellow oil, which was used in the subsequent step without further purification.

¹H NMR (400 MHz, CDCl₃) S8.48 (s, 1H), 7.99 (d, J=8.6 Hz, 1H), 7.84 (d, J=8.2 Hz, 1H), 7.64 (t, J=7.4 Hz, 1H), 7.56-7.50 (m, 1H), 7.30 (s, 1H), 6.78 (s, 1H), 6.62 (d, J=5.9 Hz, 1H), 6.31 (d, J=9.4 Hz, 1H), 5.95-5.80 (m, 1H), 5.26-5.14 (m, 3H), 4.81-4.56 (m, 3H), 4.49-4.35 (m, 4H), 4.34-4.18 (m, 4H), 4.10-4.05 (m, 2H), 4.01 (s, 3H), 3.99-3.94 (br, 2H), 3.74 (br, 4H), 3.64-3.57 (m, 2H), 2.72-2.59 (m, 4H), 2.50 (s, 3H), 2.45-2.38 (m, 3H), 1.94-1.73 (m, 9H); ¹³C NMR (100 MHz, CDCl₃) δ 170.7, 169.1, 157.8, 151.7, 149.3, 148.3, 141.0, 139.3, 132.0, 129.7, 127.6, 124.9, 124.8, 122.6, 122.4, 120.8, 117.2, 114.0, 110.8, 108.2, 106.5, 95.3, 84.1, 68.0, 66.4, 63.2, 57.7, 56.0, 54.7, 53.0, 46.6, 46.1, 45.7, 42.4, 31.9, 30.7, 29.2, 25.2, 23.8, 22.9, 18.1; MS (ES+): m/z=874 (M+H)⁺; LCMS (Method C): t_{R}=3.00 min.

### Example 58: (S)-1-(Chloromethyl)-3-(4-(((S)-2-methoxy-12-oxo-6a,7,8,9,10, 12-hexahydrobenzo[e]pyrido[1,2-a][1,4]diazepin-3-yl)oxy)butanoyl)-2,3-dihydro-1H-benzo[e]indol-5-yl 4-methylpiperazine-1-carboxylate (55)

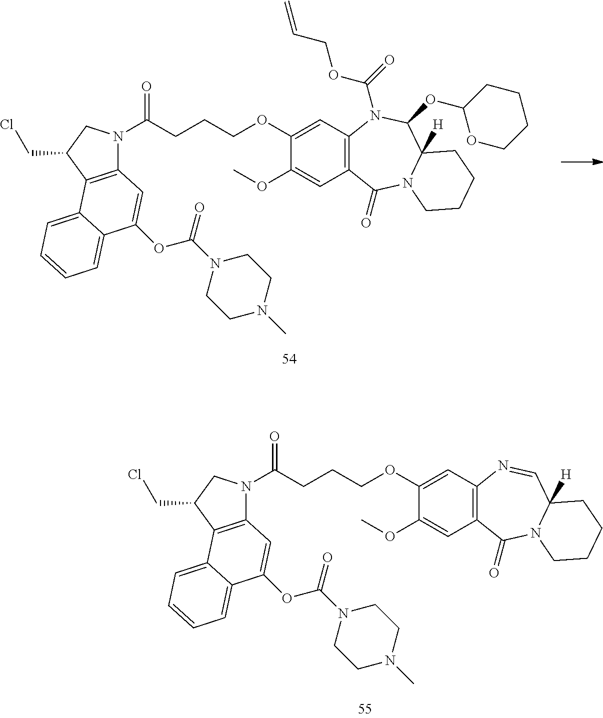 [SMILES: C=CCOC(=O)N1c2cc(OCCCC(=O)N3C[C@@H](CCl)c4c3cc(OC(=O)N3CCN(C)CC3)c3ccccc43)c(OC)cc2C(=O)N2CCCC[C@H]2[C@@H]1OC1CCCCO1 ; COc1cc2c(cc1OCCCC(=O)N1C[C@@H](CCl)c3c1cc(OC(=O)N1CCN(C)CC1)c1ccccc31)N=C[C@@H]1CCCCN1C2=O]

A solution of allyl (6S,6aS)-3-(4-((S)-1-(chloromethyl)-5-((4-methylpiperazine-1-carbonyl)oxy)-1,2-dihydro-3H-benzo[e]indol-3-yl)-4-oxobutoxy)-2-methoxy-12-oxo-6-((tetrahydro-2H-pyran-2-yl)oxy)-6,6a,7,8,9,10-hexahydrobenzo[e]pyrido[1,2-a][1,4]-diazepine-5(12H)-carboxylate (54) (95 mg, 0.11 mmol) in dichloromethane (0.5 mL) was charged with tetrakis(triphenylphosphine)palladium(0) (13 mg) and pyrrolidine (11 μL) and then stirred at room temperature under argon. After 5 min, the resulting mixture was concentrated in vacuo and purified by column chromatography (silica), eluting with ethyl acetate (100%), followed by triethylamine/ethyl acetate (3%), then triethylamine/methanol/ethyl acetate (from 3:5:95 to 3:10:90), to give the title compound (48 mg, 65%) as a creamy solid.

¹H NMR (400 MHz, acetone-d₆) δ 8.38 (s, 1H), 7.96 (d, J=5.9 Hz, 1H), 7.93-7.88 (m, 2H), 7.56-7.50 (m, 1H), 7.44-7.39 (m, 1H), 7.33 (s, 1H), 6.81 (s, 1H), 4.44-4.32 (m, 2H), 4.29-4.09 (m, 3H), 4.03 (dd, J=10.9, 3.1 Hz, 1H), 3.86 (s, 3H), 3.82-3.72 (m, 2H), 3.55 (br, 2H), 3.33-3.28 (m, 2H), 3.24-3.20 (m, 1H), 3.18-3.12 (m, 1H), 2.89-2.78 (m, 1H), 2.76-2.66 (m, 1H), 2.52 (br, 2H), 2.45 (br, 2H), 2.40-2.36 (m, 1H), 2.31 (s, 3H), 2.20 (t, J=6.6 Hz, 1H), 2.18-2.09 (m, 1H), 2.00-1.89 (m, 1H), 1.81-1.75 (m, 2H), 1.73-1.55 (m, 2H); ¹³C NMR (100 MHz, acetone-d₆) δ 170.6, 166.7, 163.9, 153.0, 150.9, 147.9, 141.6, 140.4, 130.0, 127.3, 124.6, 124.4, 122.9, 122.5, 120.9, 117.1, 111.8, 110.7, 110.0, 67.9, 55.4, 54.6, 52.8, 49.6, 47.9, 46.9, 45.7, 45.5, 45.3, 39.1, 31.5, 25.2, 24.2, 23.0, 18.2; MS (ES+): m/z=688 (M+H)⁺; LCMS (Method C): t_{R}=2.72 min.

Reaction Scheme for Preparing Compound (57)

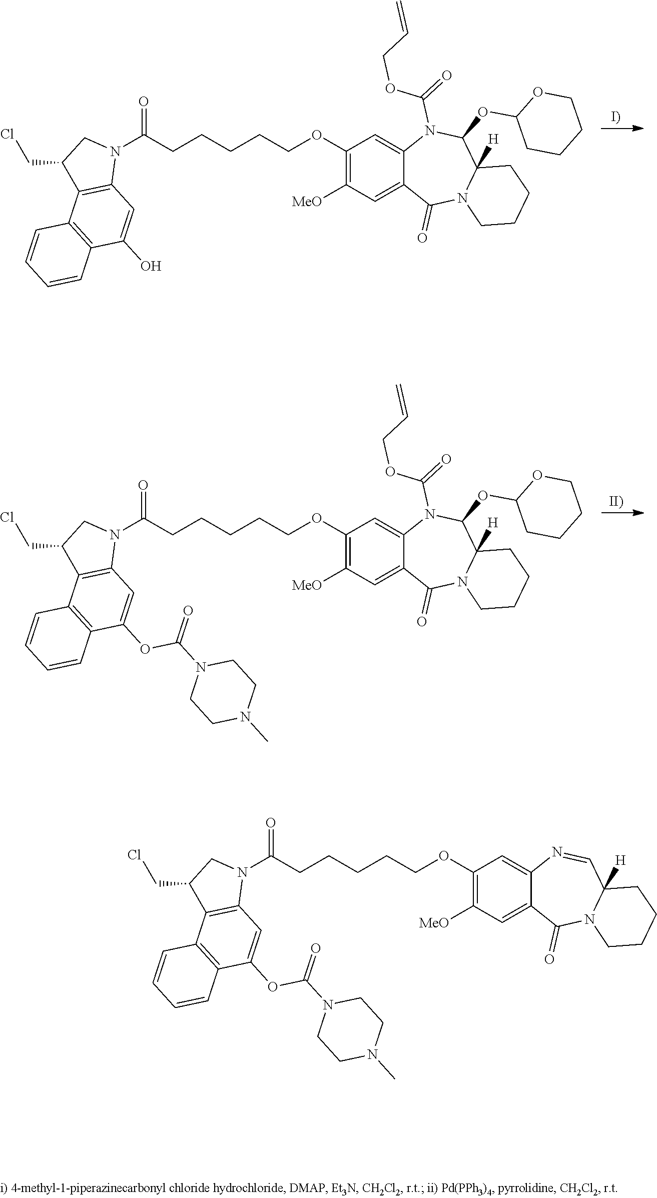

### Example 59: Allyl (6S,6aS)-3-((6-((S)-1-(chloromethyl)-5-((4-methyl-piperazine-1-carbonyl)oxy)-1,2-dihydro-3H-benzo[e]indol-3-yl)-6-oxohexyl)oxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (56)

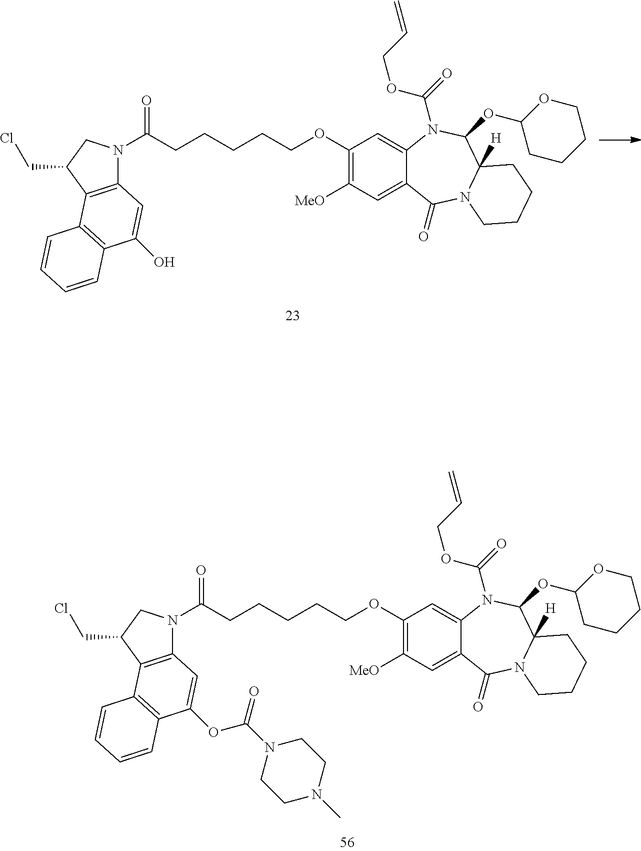 [SMILES: C=CCOC(=O)N1c2cc(OCCCCCC(=O)N3C[C@@H](CCl)c4c3cc(O)c3ccccc43)c(OC)cc2C(=O)N2CCCC[C@H]2[C@@H]1OC1CCCCO1 ; C=CCOC(=O)N1c2cc(OCCCCCC(=O)N3C[C@@H](CCl)c4c3cc(OC(=O)N3CCN(C)CC3)c3ccccc43)c(OC)cc2C(=O)N2CCCC[C@H]2[C@@H]1OC1CCCCO1]

A solution of allyl (6S,6aS)-3-((6-((S)-1-(chloromethyl)-5-hydroxy-1,2-dihydro-3H-benzo[le]indol-3-yl)-6-oxohexyl)oxy)-2-methoxy-12-oxo-6-((tetrahydro-2H-pyran-2-yl)oxy)-6,6a,7,8,9,10-hexahydrobenzo[le]pyrido[1,2-a][1,4]diazepine-5(12H)-carboxylate (23) (49 mg, 0.063 mmol) in dichloromethane (5 mL) was charged with 4-methyl-1-piperazinecarbonyl chloride hydrochloride (38 mg, 0.19 mmol), 4-(dimethyl-amino)pyridine (8.5 mg, 0.069 mmol) and triethylamine (30 μL, 0.22 mmol) and stirred at room temperature for 18 h. The reaction mixture was subsequently washed with water (2×10 mL), dried over magnesium sulfate and concentrated in vacuo, to give the title compound (45 mg, 79%) as a brown oil, which was used in the subsequent step without further purification.

¹H NMR (400 MHz, CDCl₃) δ 8.35 (s, 1H), 7.85 (d, J=8.2 Hz, 1H), 7.71 (d, J=8.2 Hz, 1H), 7.54-7-49 (m, 1H), 7.41 (t, J=70.8 Hz, 1H), 7.16 (s, 1H), 6.51 (s, 1H), 6.18 (d, J=90.4 Hz, 1H), 6.01 (d, J=10.2 Hz, 1H), 5.83-5.69 (m, 1H), 5.16-5.00 (m, 2H), 4.68-4.54 (in, 1H), 4.34-4.20 (m, 3H), 4.13-3.93 (m, 4H), 3.89 (s, 3H), 3.73 (br, 1H), 3.65 (br, 3H), 3.51-3.45 (m, 2H), 3.11-3.01 (m, 1H), 2.87 (br, 1H), 2.57 (br, 6H), 2.41 (s, 3H), 2.37-2.31 (m, 2H), 1.97-1.87 (m, 2H), 1.84-1.71 (m, 6H), 1.68-1.44 (in, 10H); ¹³C NMR (100 MHz, CDCl₃) δ 172.1, 169.2, 153.3, 149.3, 148.3, 143.9, 139.7, 133.6, 132.0, 127.6, 125.7, 124.8, 122.7, 122.4, 121.0, 120.8, 117.9, 113.7, 110.9, 108.1, 94.0, 93.6, 68.9, 66.4, 63.2, 60.1, 56.1, 54.5, 53.4, 53.1, 46.0, 42.5, 38.8, 35.7, 30.7, 28.9, 25.7, 25.3, 24.2, 23.0, 20.0, 18.2; MS (ES+): m/z=902 (M+H)⁺; LCMS (Method C): t_{R}=3.18 min.

### Example 60: (S)-1-(Chloromethyl)-3-(6-(((S)-2-methoxy-12-oxo-6a,7,8,9,10,12-hexahydrobenzo[e]pyrido[1,2-a][1,4]diazepin-3-yl)oxy)hexanoyl)-2,3-dihydro-1H-benzo[e]indol-5-yl 4-methylpiperazine-1-carboxylate (57)

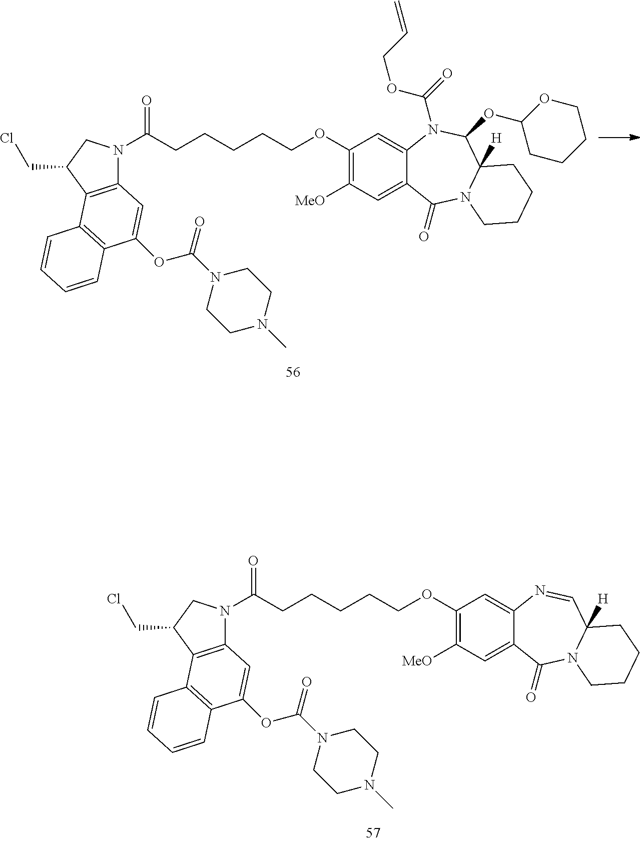 [SMILES: C=CCOC(=O)N1c2cc(OCCCCCC(=O)N3C[C@@H](CCl)c4c3cc(OC(=O)N3CCN(C)CC3)c3ccccc43)c(OC)cc2C(=O)N2CCCC[C@H]2[C@@H]1OC1CCCCO1 ; COc1cc2c(cc1OCCCCCC(=O)N1C[C@@H](CCl)c3c1cc(OC(=O)N1CCN(C)CC1)c1ccccc31)N=C[C@@H]1CCCCN1C2=O]

A solution of allyl (6S,6aS)-3-((6-((S)-1-(chloromethyl)-5-((4-methylpiperazine-1-carbonyl)oxy)-1,2-dihydro-3H-benzo[e]indol-3-yl)-6-oxohexyl)oxy)-2-methoxy-12-oxo-6-((tetrahydro-2H-pyran-2-yl)oxy)-6,6a,7,8,9,10-hexahydrobenzo[e]pyrido[1,2-a][1,4]-diazepine-5(12H)-carboxylate (56) (45 mg, 0.050 mmol) in dichloromethane (5 mL) was charged with tetrakis(triphenylphosphine)palladium(0) (6 mg) and pyrrolidine (5 μL) and then stirred at room temperature under argon. After 5 min, the resulting mixture was concentrated in vacuo and purified by column chromatography (silica), eluting with ethyl acetate (100%), followed by triethylamine/ethyl acetate (3%), then triethylamine/methanol/ethyl acetate (from 3:5:95 to 3:10:90), to give the title compound (18 mg, 50%) as a creamy solid.

¹H NMR (400 MHz, acetone-d₆) δ 8.38 (s, 1H), 7.97 (d, J=5.5 Hz, 1H), 7.92 (dd, J=8.4, 4.1 Hz, 2H), 7.55 (t, J=70.6 Hz, 1H), 7.45-7.40 (m, 1H), 7.33 (s, 1H), 6.77 (s, 1H), 4.46-4.35 (m, 2H), 4.27 (br, 1H), 4.17-4.10 (m, 2H), 4.08-4.02 (m, 2H), 3.86 (s, 3H), 3.85-3.79 (m, 2H), 3.80-3.74 (m, 2H), 3.56 (br s, 2H), 3.19-3.11 (m, 1H), 2.70-2.62 (m, 1H), 2.54 (br s, 2H), 2.46 (br s, 2H), 2.32 (s, 3H), 2.18-2.10 (m, 1H), 1.93-1.86 (m, 2H), 1.85-1.76 (m, 6H), 1.69-1.59 (m, 4H); ¹³C NMR (100 MHz, acetone-d₆) δ 172.6, 168.7, 163.9, 153.0, 151.0, 147.9, 147.0, 142.7, 140.4, 132.5, 127.3, 124.3, 122.9, 122.5, 121.2, 117.3, 116.7, 111.8, 110.0, 109.8, 68.5, 55.4, 52.8, 49.6, 49.2, 47.9, 46.9, 46.5, 45.4, 39.1, 35.1, 31.9, 25.5, 24.2, 24.0, 23.0, 18.2; MS (ES+): m/z=716 (M+H)⁺; LCMS (Method C): t_{R}=2.83 min.

Reaction Scheme for Preparing Compound (59)

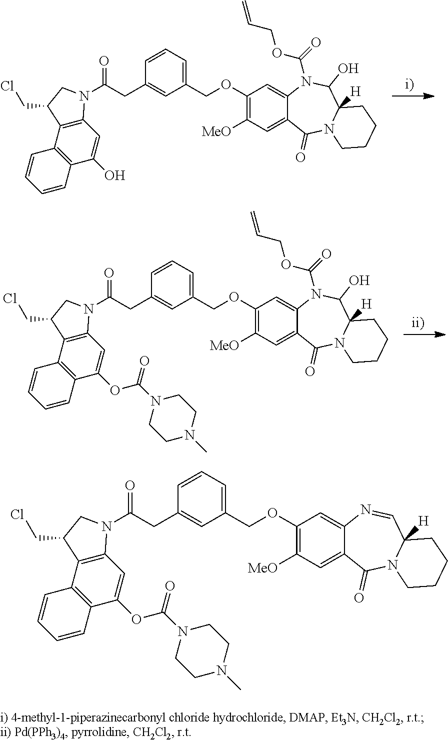

### Example 61: Allyl (6aS)-3-((3-(2-((S)-1-(chloromethyl)-5-((4-methyl-piperazine-1-carbonyl)oxy)-1,2-dihydro-3H-benzo[e]indol-3-yl)-2-oxo-ethyl)benzyl)oxy)-6-hydroxy-2-methoxy-12-oxo-6,6a,7,8,9,10-hexahydro-benzo[e]pyrido[1,2-a][1,4]diazepine-5(12H)-carboxylate (58)

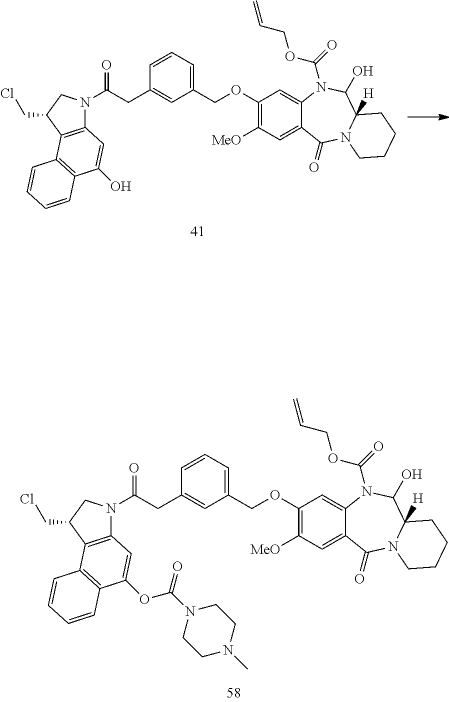 [SMILES: C=CCOC(=O)N1c2cc(OCc3cccc(CC(=O)N4C[C@@H](CCl)c5c4cc(O)c4ccccc54)c3)c(OC)cc2C(=O)N2CCCC[C@H]2C1O ; C=CCOC(=O)N1c2cc(OCc3cccc(CC(=O)N4C[C@@H](CCl)c5c4cc(OC(=O)N4CCN(C)CC4)c4ccccc54)c3)c(OC)cc2C(=O)N2CCCC[C@H]2C1O]

A solution of allyl (6aS)-3-((3-(2-((S)-1-(chloromethyl)-5-hydroxy-1,2-dihydro-3H-benzo [e]indol-3-yl)-2-oxoethyl)benzyl)oxy)-6-hydroxy-2-methoxy-12-oxo-6,6a,7,8,9,10-hexahydrobenzo[e]pyrido[1,2-a][1,4]diazepine-5(12H)-carboxylate (41) (65 mg, 0.090 mmol) in dichloromethane (5 mL) was charged with 4-methyl-1-piperazinecarbonyl chloride hydrochloride (54 mg, 0.27 mmol), 4-(dimethylamino)-pyridine (12 mg, 0.098 mmol) and triethylamine (41 μL, 0.31 mmol) and stirred at room temperature for 18 h. The reaction mixture was subsequently washed with water (2×10 mL), dried over magnesium sulfate and concentrated in vacuo, to give the title compound (50 mg, 66%) as a brown oil, which was used in the subsequent step without further purification.

¹H NMR (400 MHz, CDCl₃) δ 8.27 (s, 1H), 7.79 (d, J=8.6 Hz, 1H), 7.62 (d, J=8.2 Hz, 1H), 7.43 (t, J=70.6 Hz, 1H), 7.38-7.32 (m, 1H), 7.31-7.26 (m, 3H), 7.23-7.17 (m, 1H), 7.10 (s, 1H), 6.64 (s, 1H), 5.84 (d, J=10.2 Hz, 1H), 5.70-5.53 (m, 1H), 5.04 (br, 4H), 4.27 (d, J=9.4 Hz, 2H), 4.15-4.08 (m, 1H), 3.95 (br s, 1H), 3.80 (s, 3H), 3.69-3.64 (m, 1H), 3.57 (br s, 4H), 3.41-3.30 (m, 2H), 3.02-2.92 (m, 1H), 2.47 (br s, 4H), 2.39-2.35 (m, 3H), 2.32 (s, 3H), 1.98-1.89 (m, 2H), 1.74-1.60 (m, 2H), 1.59-1.53 (m, 3H); ¹³C NMR (100 MHz, CDCl₃) δ 169.2, 168.9, 153.3, 149.6, 149.0, 148.9, 148.2, 140.8, 140.7, 136.8, 134.1, 131.9, 129.6, 129.0, 128.9, 127.9, 127.5, 126.0, 124.9, 124.8, 122.5, 121.2, 117.6, 116.2, 114.6, 110.9, 110.6, 106.3, 101.4, 82.2, 70.8, 66.4, 56.0, 55.5, 54.5, 54.0, 53.1, 48.4, 45.9, 45.6, 38.6, 29.6, 22.9, 18.2; MS (ES+): m/z=852 (M+H)⁺; LCMS (Method C): t_{R}=2.97 min.

### Example 62: (S)-1-(Chloromethyl)-3-(2-(3-((((S)-2-methoxy-12-oxo-6a,7,8,9,10,12-hexahydrobenzo[e]pyrido[1,2-a][1,4]diazepin-3-yl)oxy)-methyl)phenyl)acetyl)-2,3-dihydro-1H-benzo[e]indol-5-yl 4-methyl-piperazine-1-carboxylate (59)

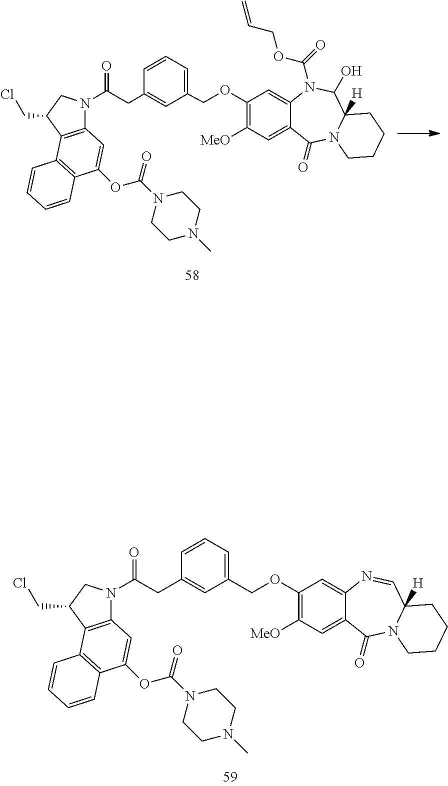 [SMILES: C=CCOC(=O)N1c2cc(OCc3cccc(CC(=O)N4C[C@@H](CCl)c5c4cc(OC(=O)N4CCN(C)CC4)c4ccccc54)c3)c(OC)cc2C(=O)N2CCCC[C@H]2C1O ; COc1cc2c(cc1OCc1cccc(CC(=O)N3C[C@@H](CCl)c4c3cc(OC(=O)N3CCN(C)CC3)c3ccccc43)c1)N=C[C@@H]1CCCCN1C2=O]

A solution of allyl (6aS)-3-((3-(2-((S)-1-(chloromethyl)-5-((4-methylpiperazine-1-carbonyl)oxy)-1,2-dihydro-3H-benzo [e]indol-3-yl)-2-oxoethyl)benzyl)oxy)-6-hydroxy-2-methoxy-12-oxo-6,6a,7,8,9,10-hexahydrobenzo[e]pyrido[1,2-a][1,4]diazepine-5(12H)-carboxylate (58) (50 mg, 0.059 mmol) in dichloromethane (5 mL) was charged with tetrakis(triphenylphosphine)palladium(0) (7 mg) and pyrrolidine (6 μL) and then stirred at room temperature under argon. After 5 min, the resulting mixture was concentrated in vacuo and purified by column chromatography (silica), eluting with ethyl acetate (100%), followed by triethylamine/ethyl acetate (3%), then triethylamine/methanol/ethyl acetate (from 3:5:95 to 3:10:90), to give the title compound (12 mg, 27%) as a creamy solid.

¹H NMR (400 MHz, acetone-d₆) δ 8.35 (s, 1H), 7.96-7.90 (m, 3H), 7.59-7.52 (m, 2H), 7.45 (d, J=8.6 Hz, 1H), 7.41 (d, J=30.5 Hz, 1H), 7.37 (br, 2H), 7.34 (d, J=2.0 Hz, 1H), 6.85 (d, J=5-5 Hz, 1H), 5.26-5.25 (m, 2H), 4.51-4.38 (m, 2H), 4.26 (br, 1H), 4.13 (d, J=12.1 Hz, 1H), 4.01 (br, 4H), 3.89-3.82 (m, 5H), 3.80-3.70 (m, 4H), 3.55 (br, 4H), 2.53 (br, 2H), 2.46 (br, 2H), 2.31 (s, 3H), 1.66-1.57 (m, 2H); ¹³C NMR (100 MHz, acetone-d₆) δ 168.1, 164.0, 157.4, 153.3, 151.3, 150.5, 148.0, 146.4, 143.4, 140.2, 135.3, 132.8, 129.1, 128.6, 127.4, 126.0, 124.5, 123.6, 123.0, 122.5, 118.4, 116.6, 111.8, 110.7, 110.6, 70.3, 55.4, 54.5, 53.1, 49.5, 47.9, 46.7, 45.6, 45.4, 45.3, 39.1, 25.2, 24.1, 18.2; MS (ES+): m/z=750 (M+H)⁺; LCMS (Method C): t_{R}=2.90 min; HRMS calculated for [C₄₂H₄₅ClN₅O₆]⁺: 750.3053, found: 750.3033.

Reaction Scheme for Preparing Compound (64)

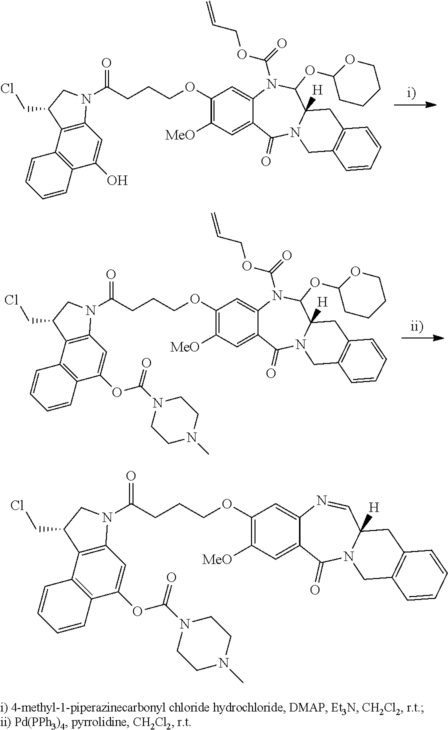 [SMILES: C=CCOC(=O)N1c2cc(OCCCC(=O)N3C[C@@H](CCl)c4c3cc(O)c3ccccc43)c(OC)cc2C(=O)N2Cc3ccccc3C[C@H]2C1OC1CCCCO1 ; C=CCOC(=O)N1c2cc(OCCCC(=O)N3C[C@@H](CCl)c4c3cc(OC(=O)N3CCN(C)CC3)c3ccccc43)c(OC)cc2C(=O)N2Cc3ccccc3C[C@H]2C1OC1CCCCO1 ; COc1cc2c(cc1OCCCC(=O)N1C[C@@H](CCl)c3c1cc(OC(=O)N1CCN(C)CC1)c1ccccc31)N=C[C@@H]1Cc3ccccc3CN1C2=O]

### Example 63: Allyl (6aS)-3-(4-((S)-1-(chloromethyl)-5-((4-methylpiperazine-1-carbonyl)oxy)-1,2-dihydro-3H-benzo[e]indol-3-yl)-4-oxobutoxy)-2-methoxy-14-oxo-6-((tetrahydro-2H-pyran-2-yl)oxy)-6,6a,7,12-tetrahydro-benzo[5,6][1,4]diazepino[1,2-b]isoquinoline-5(14H)-carboxylate (60)

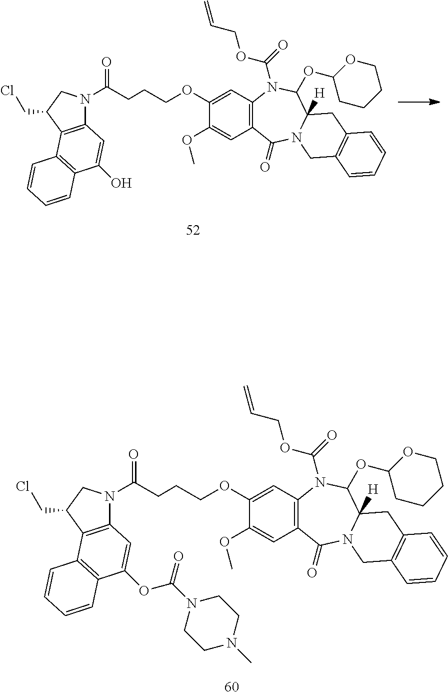 [SMILES: C=CCOC(=O)N1c2cc(OCCCC(=O)N3C[C@@H](CCl)c4c3cc(O)c3ccccc43)c(OC)cc2C(=O)N2Cc3ccccc3C[C@H]2C1OC1CCCCO1 ; C=CCOC(=O)N1c2cc(OCCCC(=O)N3C[C@@H](CCl)c4c3cc(OC(=O)N3CCN(C)CC3)c3ccccc43)c(OC)cc2C(=O)N2Cc3ccccc3C[C@H]2C1OC1CCCCO1]

A solution of allyl (6aS)-3-(4-((S)-1-(chloromethyl)-5-hydroxy-1,2-dihydro-3H-benzo-[e]indol-3-yl)-4-oxobutoxy)-2-methoxy-14-oxo-6-((tetrahydro-2H-pyran-2-yl)oxy)-6,6a,7,12-tetrahydrobenzo[5,6][1,4]diazepino[1,2-b]isoquinoline-5(14H)-carboxylate (52) (30 mg, 0.038 mmol) in dichloromethane (4 mL) was charged with 4-methyl-1-piperazinecarbonyl chloride hydrochloride (22.5 mg, 0.113 mmol), 4-(dimethylamino)-pyridine (5 mg, 0.042 mmol) and triethylamine (17 μL, 0.13 mmol) and stirred at room temperature for 18 h. The reaction mixture was subsequently washed with water (2×10 mL), dried over magnesium sulfate and concentrated in vacuo, to give the title compound (27 mg, 77%) as a brown oil, which was used in the subsequent step without further purification.

¹H NMR (400 MHz, acetone-d₆) δ 8.36 (d, J=2.7 Hz, 1H), 7.97-7.92 (m, 2H), 7.93-7.87 (m, 1H), 7.57-7.51 (m, 1H), 7.44-7.39 (m, 1H), 7.34-7.28 (m, 3H), 7.14 (d, J=70.8 Hz, 1H), 6.98 (s, 1H), 5.83-5.67 (m, 1H), 5.64-5.56 (m, 1H), 5.42 (d, J=9.4 Hz, 1H), 5.12-4.96 (m, 2H), 4.76-4.66 (m, 1H), 4.51-4.40 (m, 2H), 4.39-4.25 (m, 3H), 4.17 (br, 2H), 4.08-4.02 (m, 1H), 4.00-3.91 (m, 1H), 3.86 (s, 3H), 3.84-3.77 (m, 2H), 3.68-3.48 (m, 4H), 3.20-3.06 (m, 3H), 2.89-2.79 (m, 1H), 2.57-2.42 (m, 4H), 2.30 (s, 3H), 2.23-2.15 (m, 2H), 2.06-2.01 (m, 2H), 1.80-1.66 (m, 2H), 1.61-1.43 (m, 4H); ¹³C NMR (100 MHz, acetone-d₆) δ 171.1, 161.8, 153.0, 149.2, 149.0, 147.2, 144.6, 143.5, 136.0, 135.4, 134.8, 127.9, 127.7, 127.6, 127.3, 126.9, 126.8, 126.4, 123.0, 122.9, 122.5, 120.9, 120.7, 116.4, 114.8, 110.7, 109.6, 107.3, 102.8, 97.2, 67.9, 65.8, 63.2, 59.5, 55.5, 52.8, 49.6, 49.1, 46.9, 45.4, 43.5, 41.9, 35.2, 33.7, 31.5, 30.9, 30.1, 25.2; MS (ES+): m/z=922 (M+H)⁺; LCMS (Method C): t_{R}=3.20 min.

### Example 64: (S)-1-(Chloromethyl)-3-(4-(((S)-2-methoxy-14-oxo-6a,7,12,14-tetrahydrobenzo[5,6][1,4]diazepino[1,2-b]isoquinolin-3-yl)oxy)butanoyl)-2,3-dihydro-1H-benzo[e]indol-5-yl 4-methylpiperazine-1-carboxylate (61)

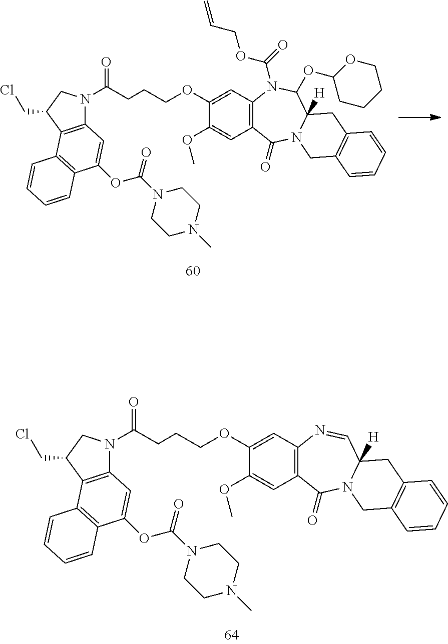 [SMILES: C=CCOC(=O)N1c2cc(OCCCC(=O)N3C[C@@H](CCl)c4c3cc(OC(=O)N3CCN(C)CC3)c3ccccc43)c(OC)cc2C(=O)N2Cc3ccccc3C[C@H]2C1OC1CCCCO1 ; COc1cc2c(cc1OCCCC(=O)N1C[C@@H](CCl)c3c1cc(OC(=O)N1CCN(C)CC1)c1ccccc31)N=C[C@@H]1Cc3ccccc3CN1C2=O]

A solution of allyl (6aS)-3-(4-((S)-1-(chloromethyl)-5-((4-methylpiperazine-1-carbonyl)oxy)-1,2-dihydro-3H-benzo[e]indol-3-yl)-4-oxobutoxy)-2-methoxy-14-oxo-6-((tetrahydro-2H-pyran-2-yl)oxy)-6,6a,7,12-tetrahydrobenzo[5,6][1,4]diazepino[1,2-b]isoquinoline-5(14H)-carboxylate (60) (27 mg, 0.029 mmol) in dichloromethane (4 mL) was charged with tetrakis(triphenylphosphine)palladium(0) (3.3 mg) and pyrrolidine (3 μL) and then stirred at room temperature under argon. After 5 min, the resulting mixture was concentrated in vacuo and purified by column chromatography (silica), eluting with ethyl acetate (100%), followed by triethylamine/ethyl acetate (3%), then triethylamine/methanol/ethyl acetate (from 3:5:95 to 3:10:90), to give the title compound (9 mg, 41%) as a brown solid.

¹H NMR (400 MHz, acetone-d₆) δ 8.38 (s, 1H), 7.96-7.89 (m, 3H), 7.56 (t, J=70.6 Hz, 1H), 7.49 (t, J=4.5 Hz, 1H), 7.47-7.42 (m, 2H), 7.36-7.27 (m, 3H), 6.84 (s, 1H), 4.88 (d, J=15.2, 1H), 4.56 (d, J=15.2 Hz, 1H), 4.48-4.36 (m, 3H), 4.33-4.19 (m, 3H), 4.15-4.03 (m, 2H), 3.94-3.89 (m, 1H), 3.87 (s, 3H), 3.84-3.76 (m, 4H), 3.55 (br, 2H), 3.32 (t, J=5.3 Hz, 1H), 2.57-2.41 (m, 4H), 2.31 (s, 3H), 2.27-2.19 (m, 2H); ¹³C NMR (100 MHz, acetone-d₆) δ 170.6, 165.6, 162.4, 153.0, 149.0, 151.1, 147.9, 145.8, 136.5, 135.3, 134.4, 128.8, 127.9, 127.7, 127.0, 126.2, 124.4, 122.9, 122.5, 112.2, 116.0, 113.9, 112.2, 110.7, 110.3, 67.9, 55.4, 54.5, 52.9, 49.9, 49.4, 45.5, 43.2, 41.9, 31.5, 30.2, 24.1; MS (ES+): m/z=736 (M+H)⁺; LCMS (Method C): t_{R}=2.88 min.

Further Reaction Schemes

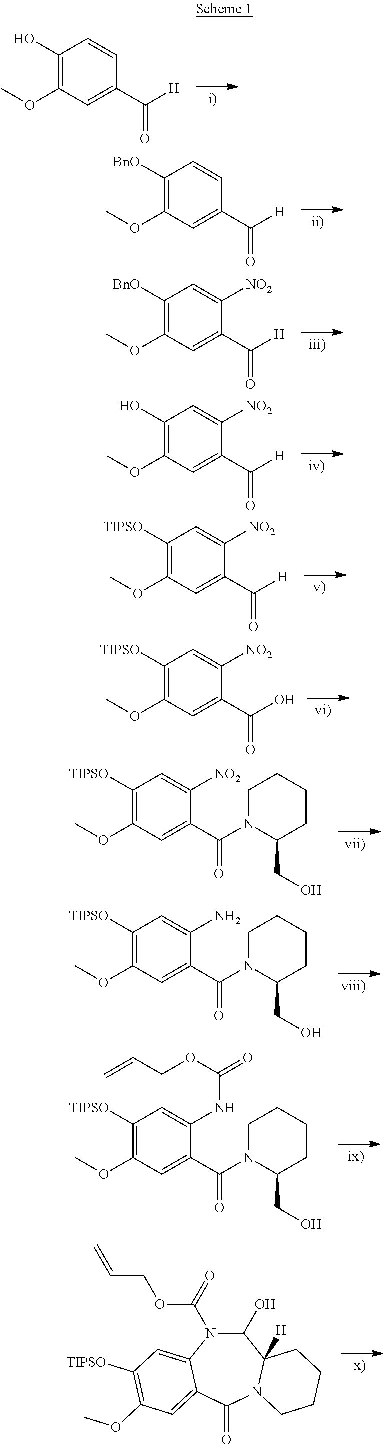

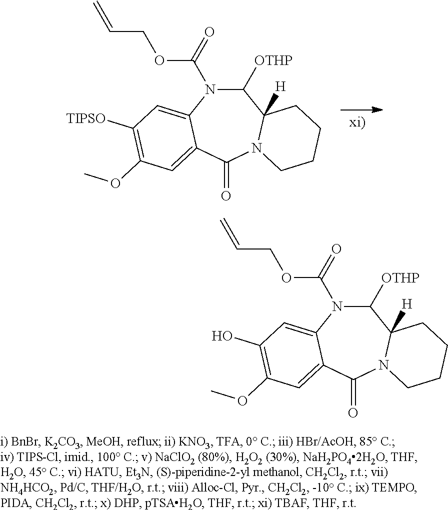

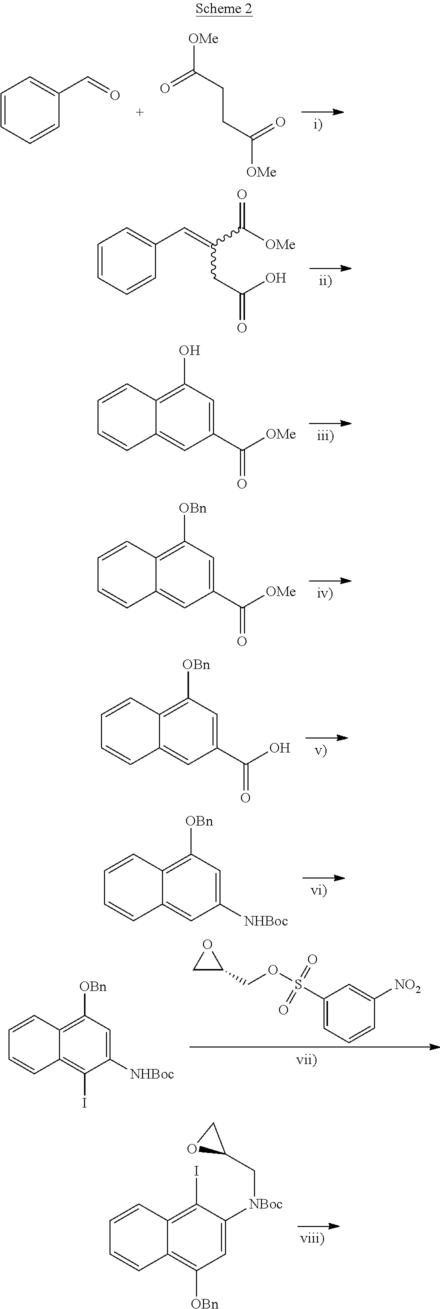

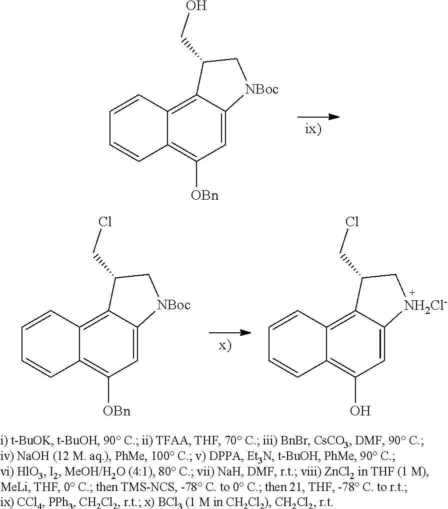

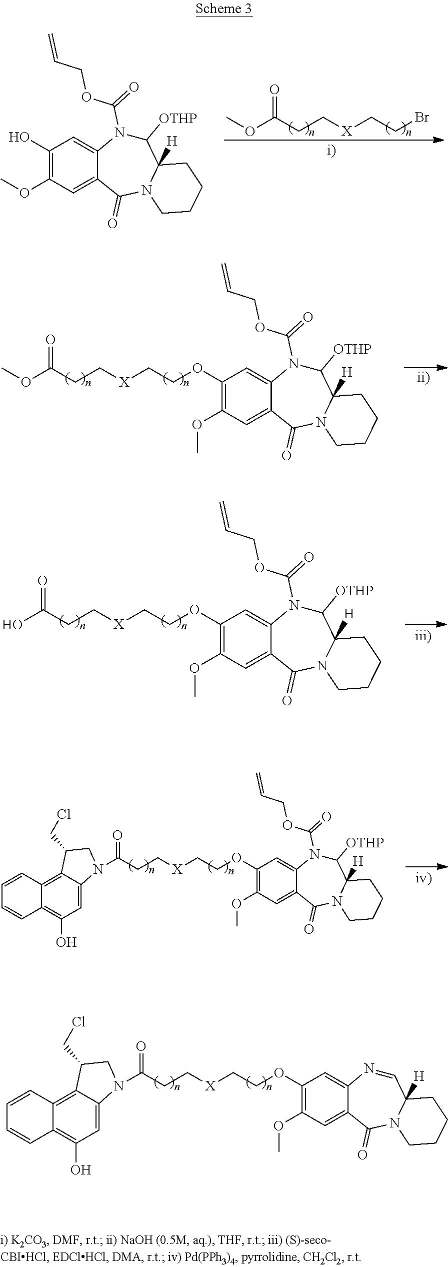

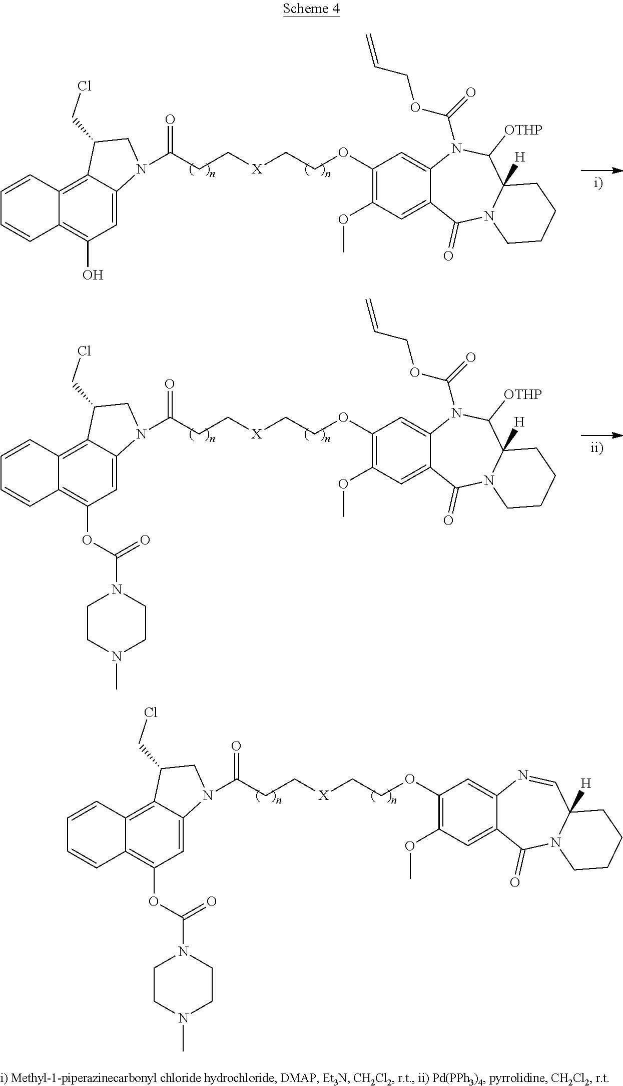

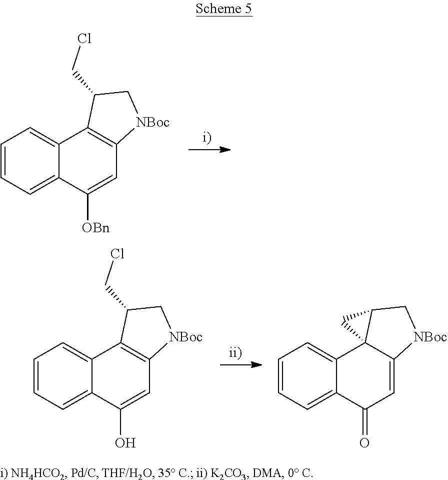

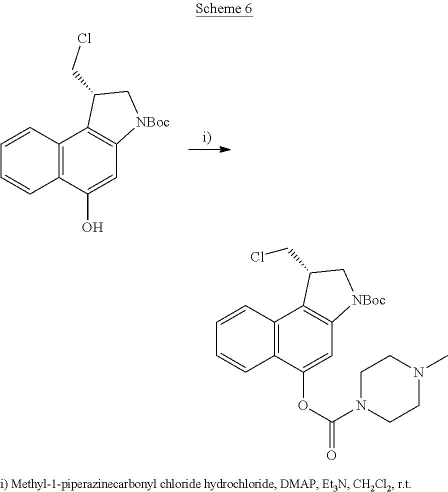 [SMILES: CC(C)(C)OC(=O)N1C[C@@H](CCl)c2c1cc(O)c1ccccc21 ; CN1CCN(C(=O)Oc2cc3c(c4ccccc24)[C@H](CCl)CN3C(=O)OC(C)(C)C)CC1]

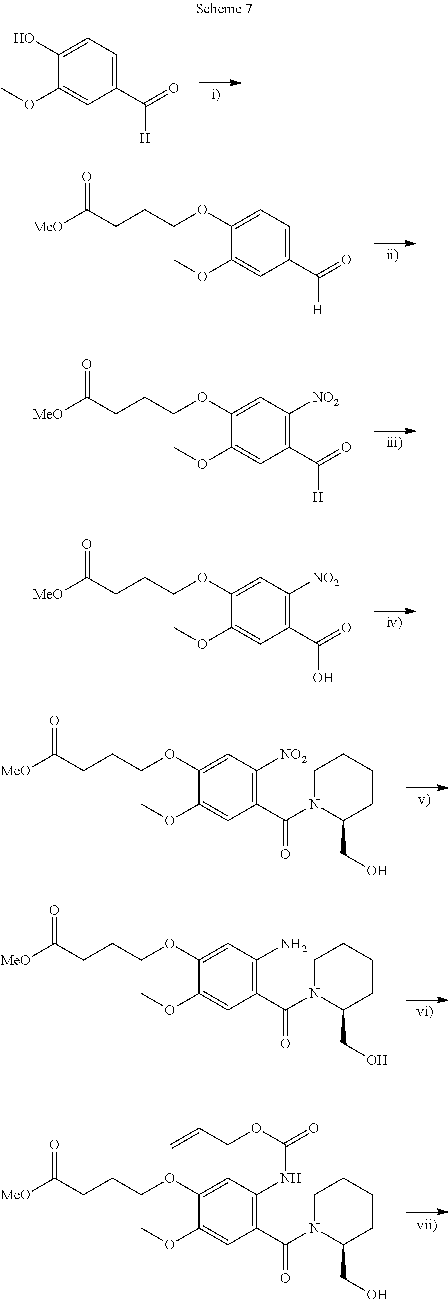

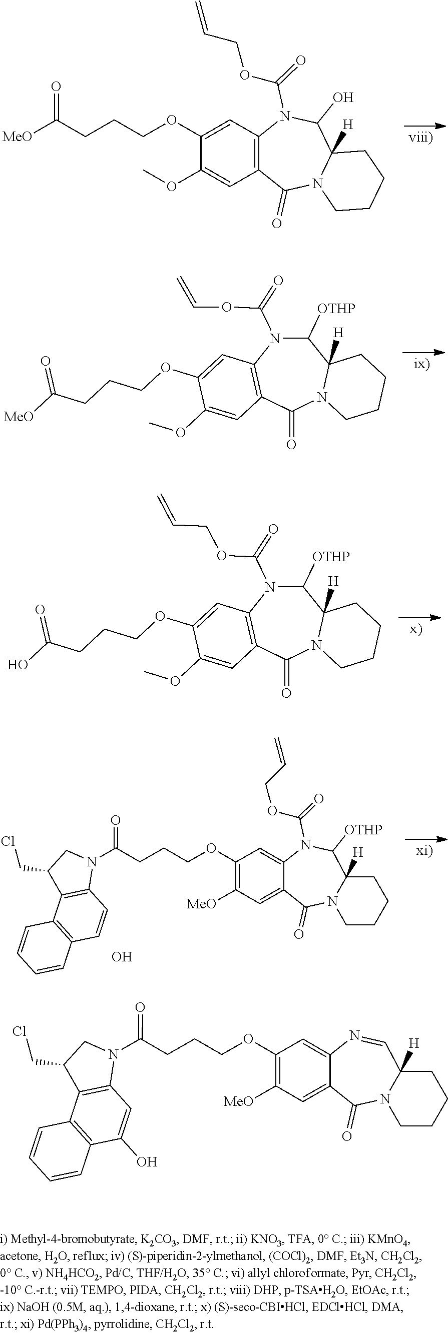

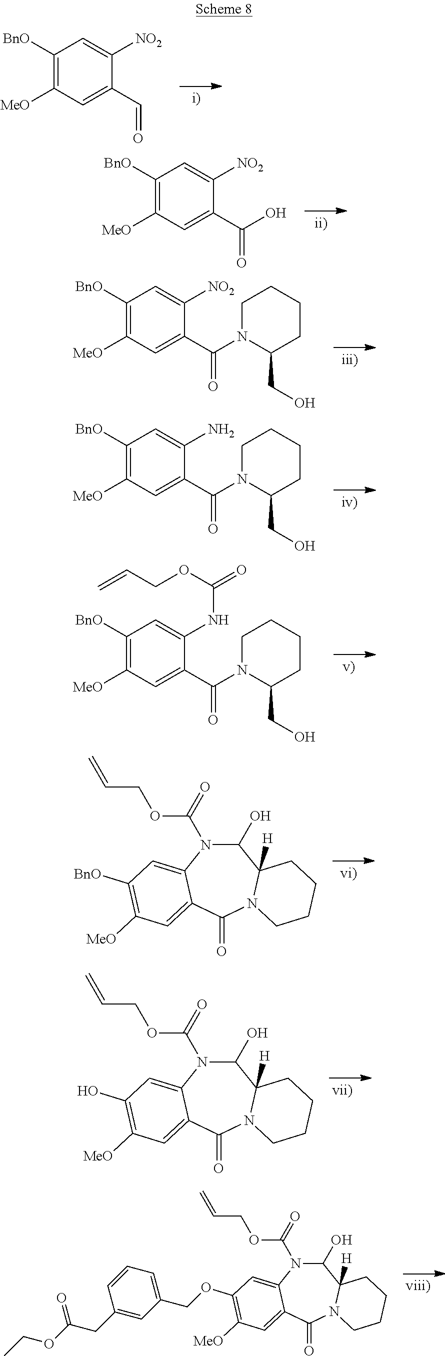

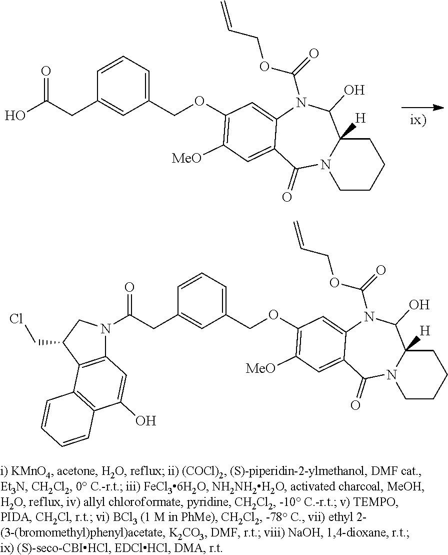

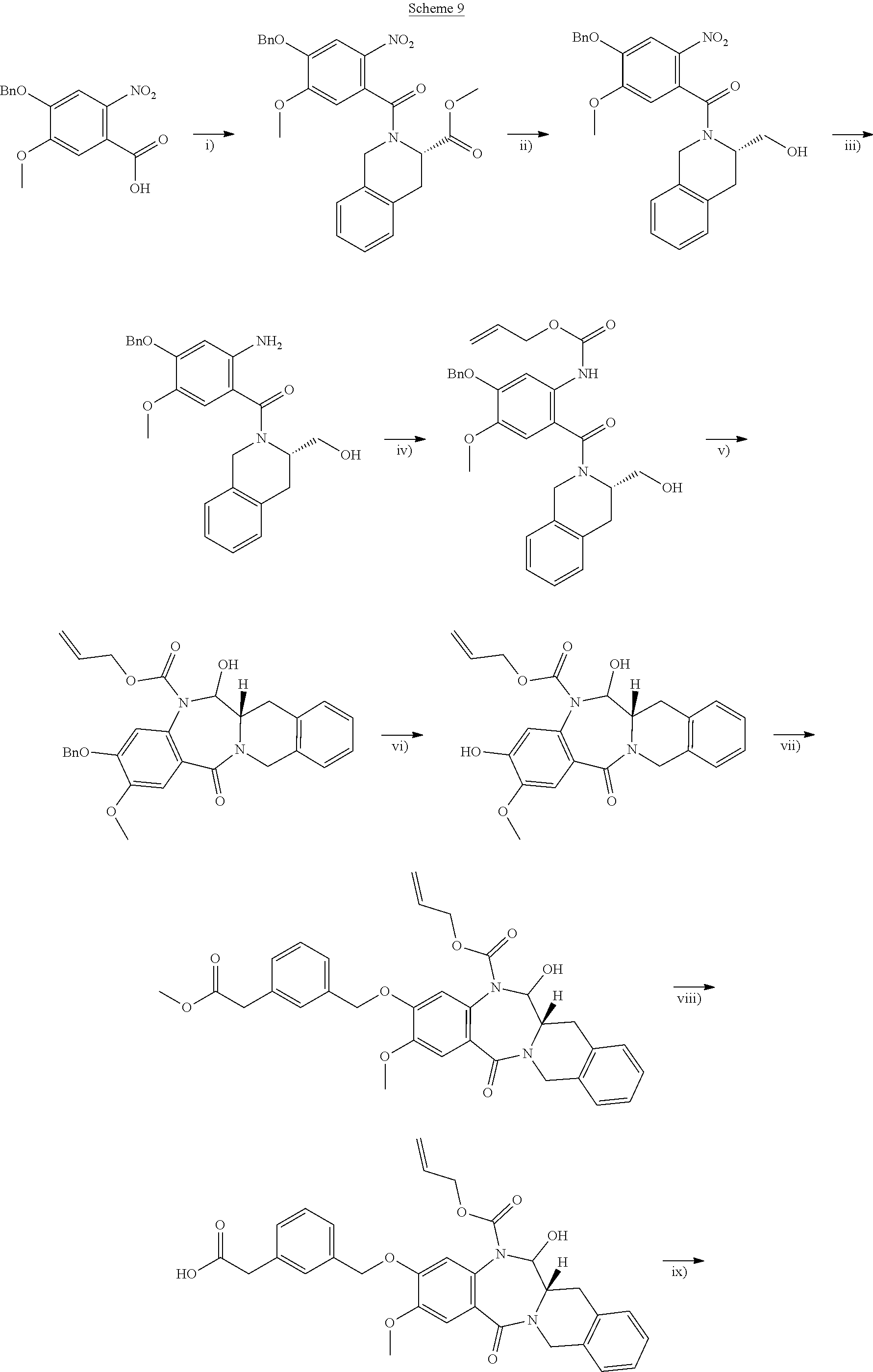

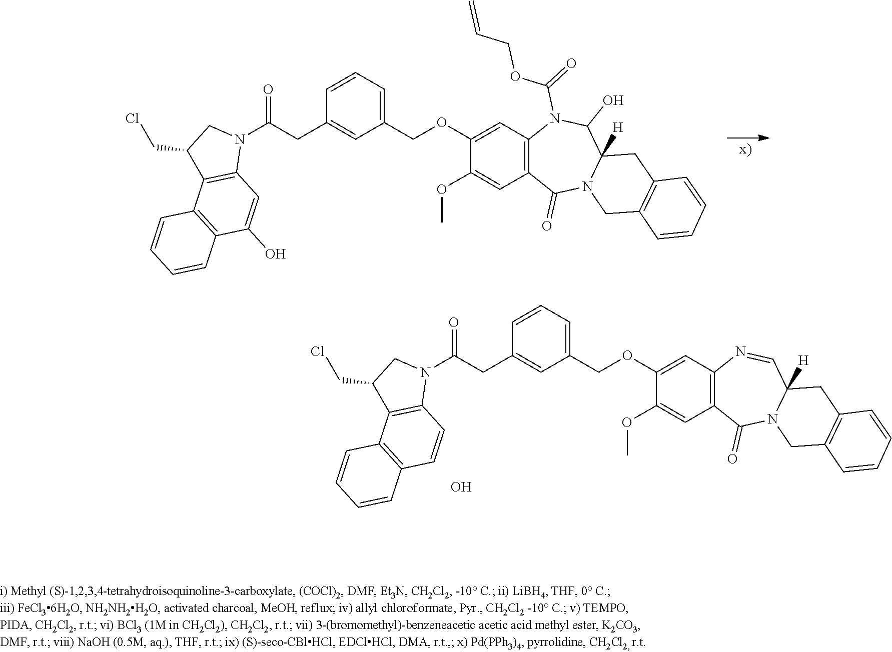

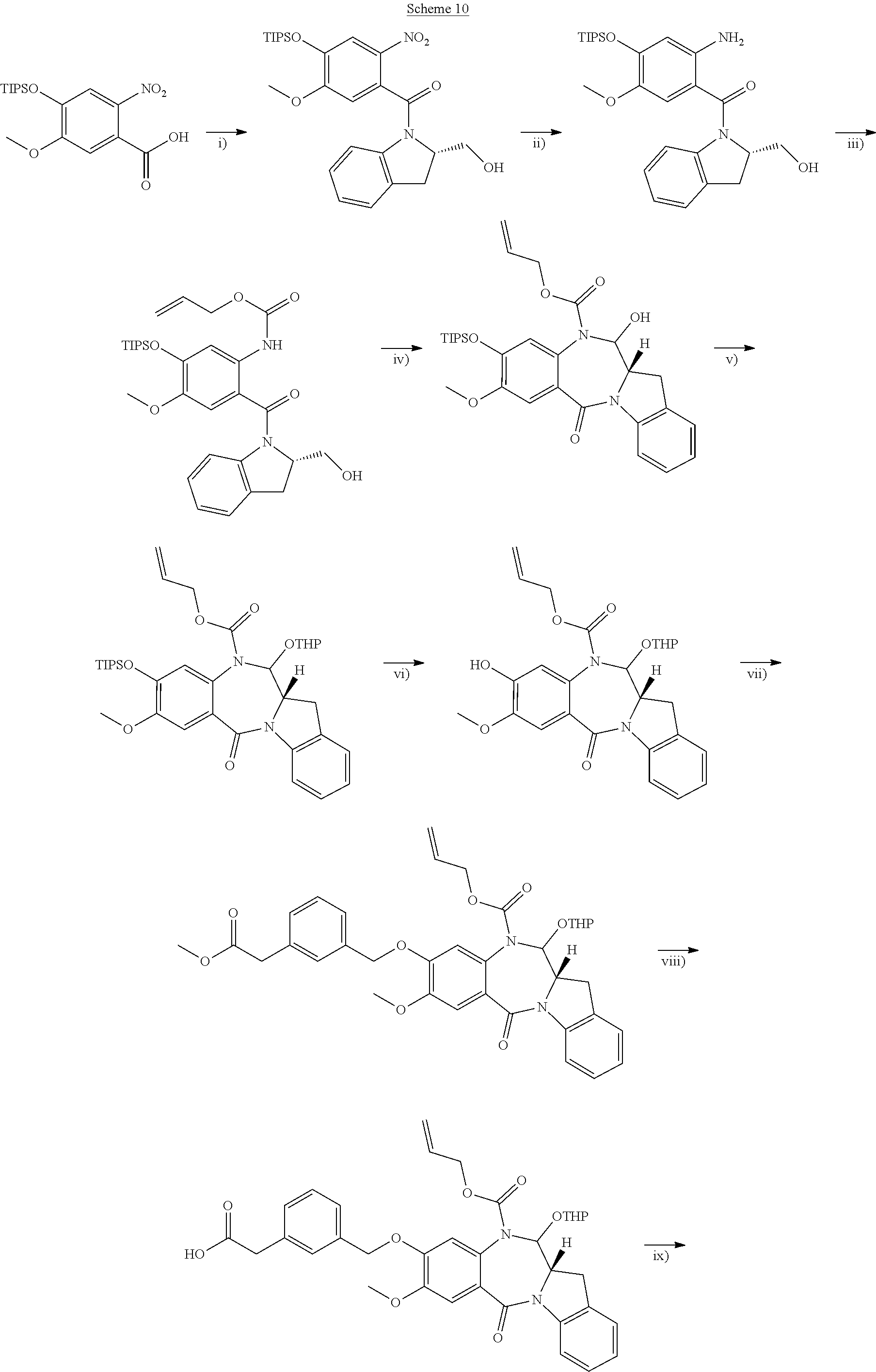

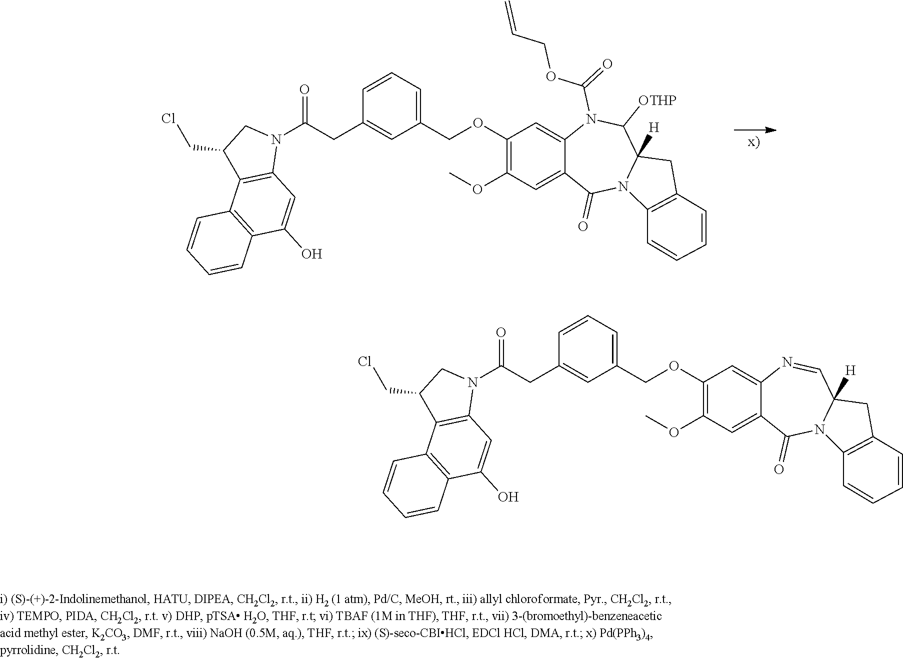

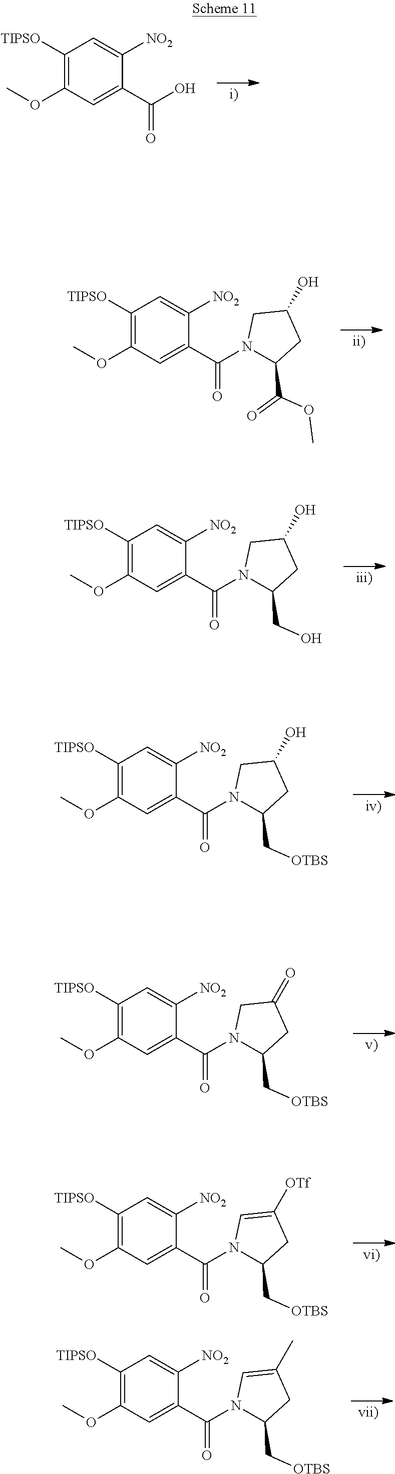

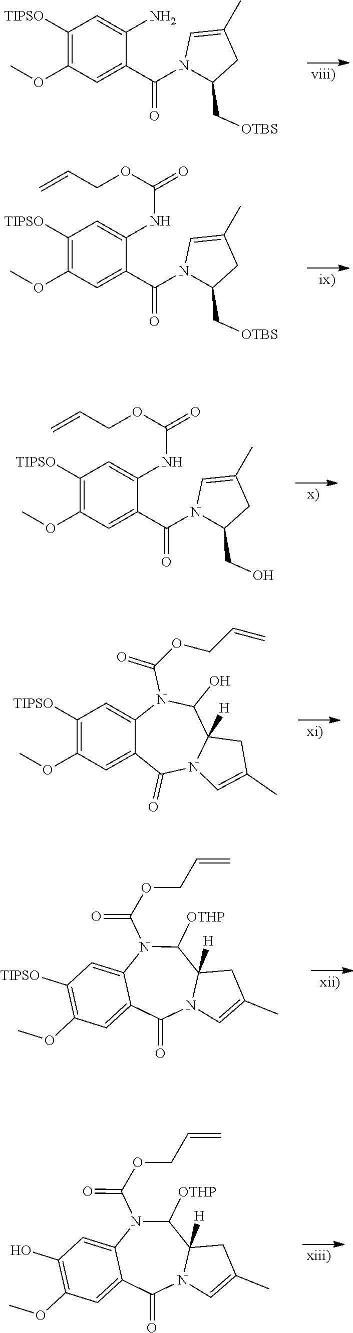

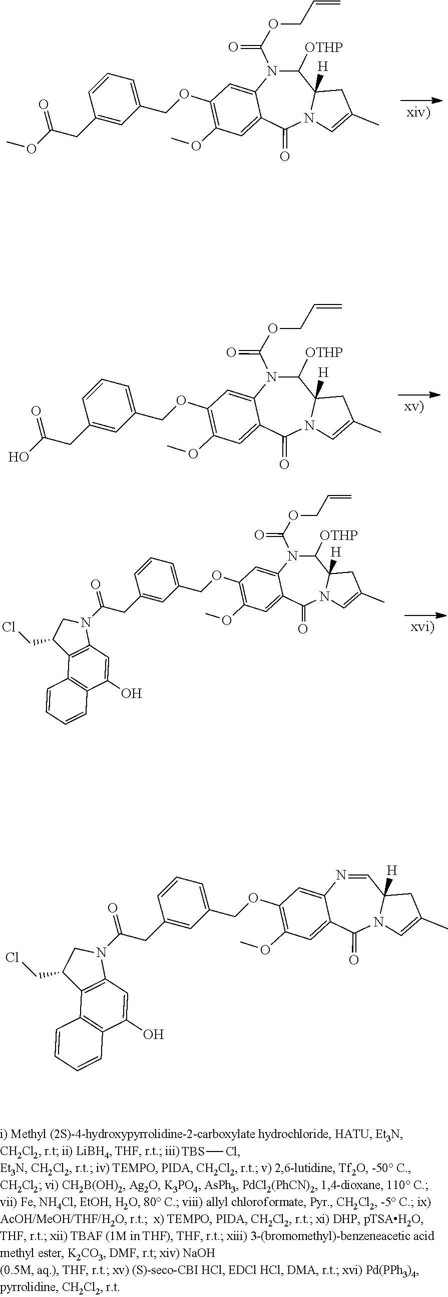

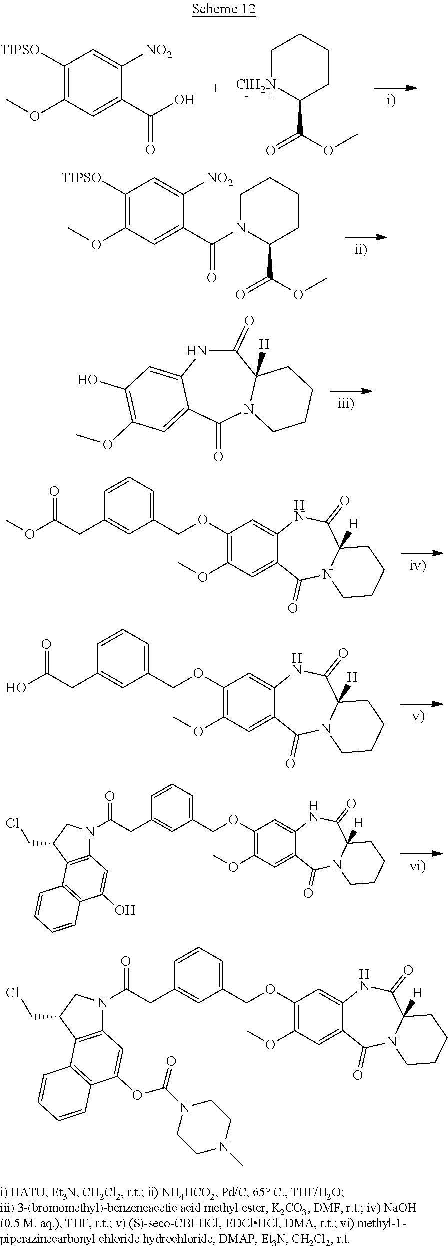

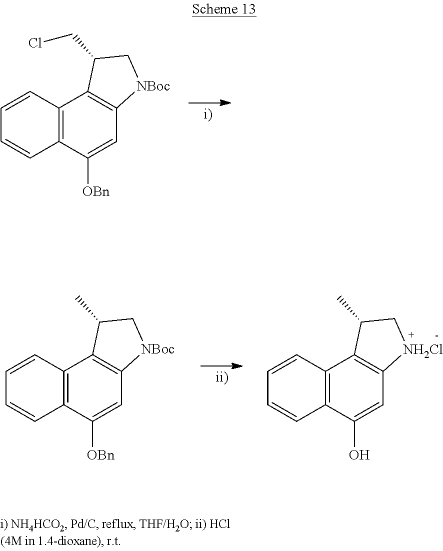

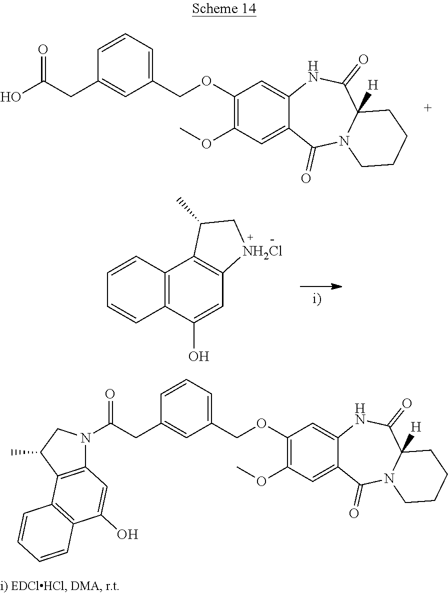

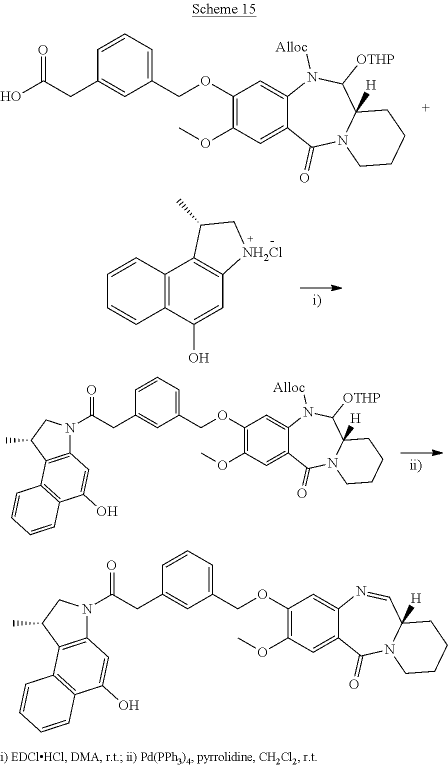

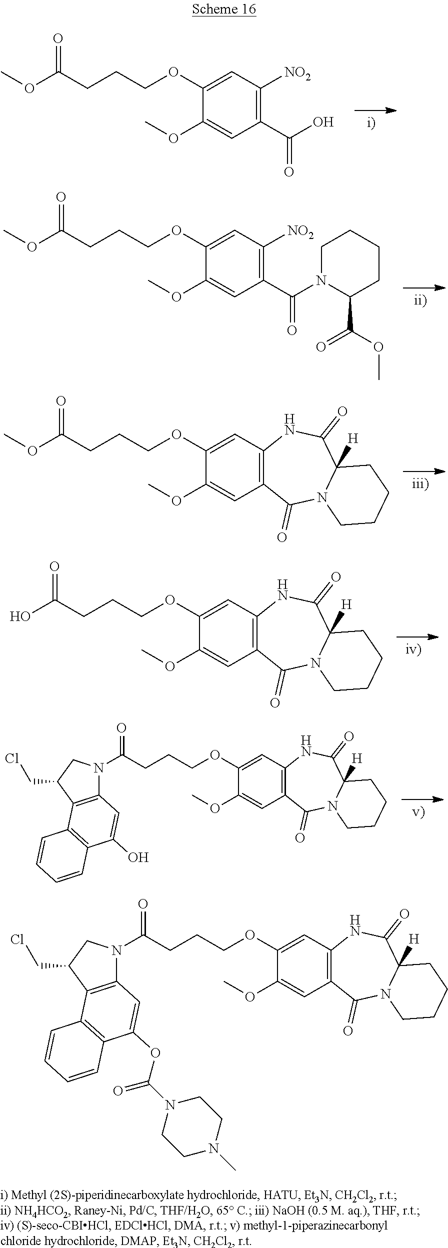

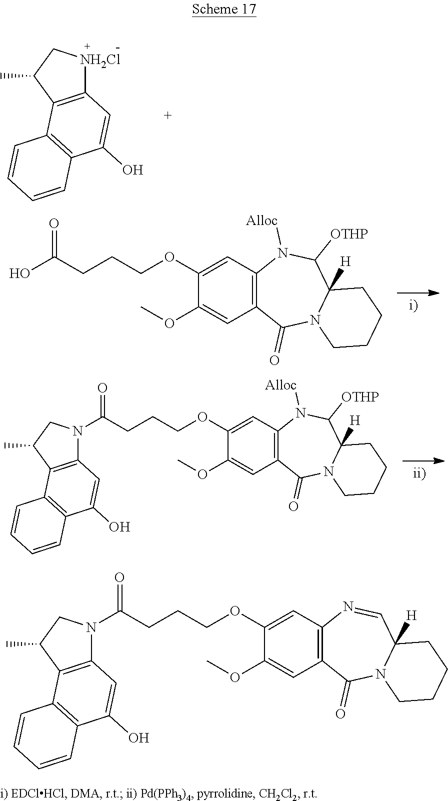

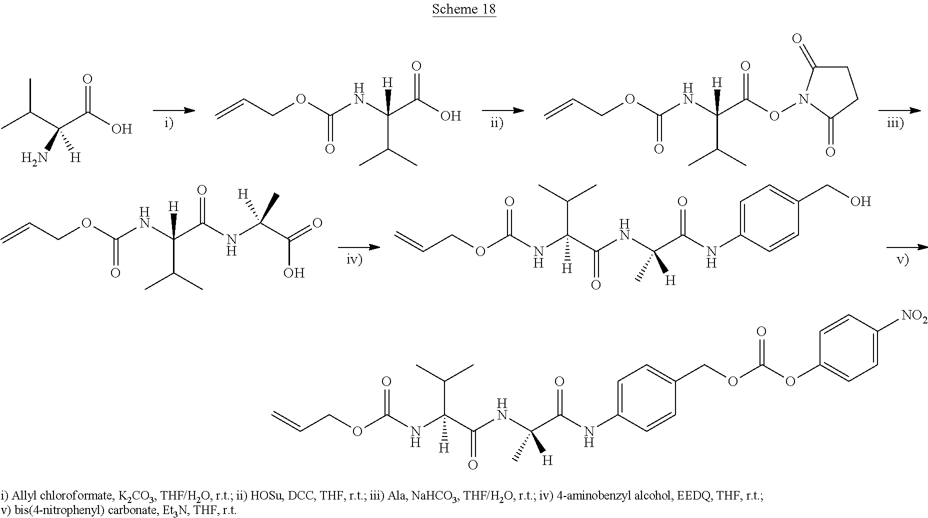

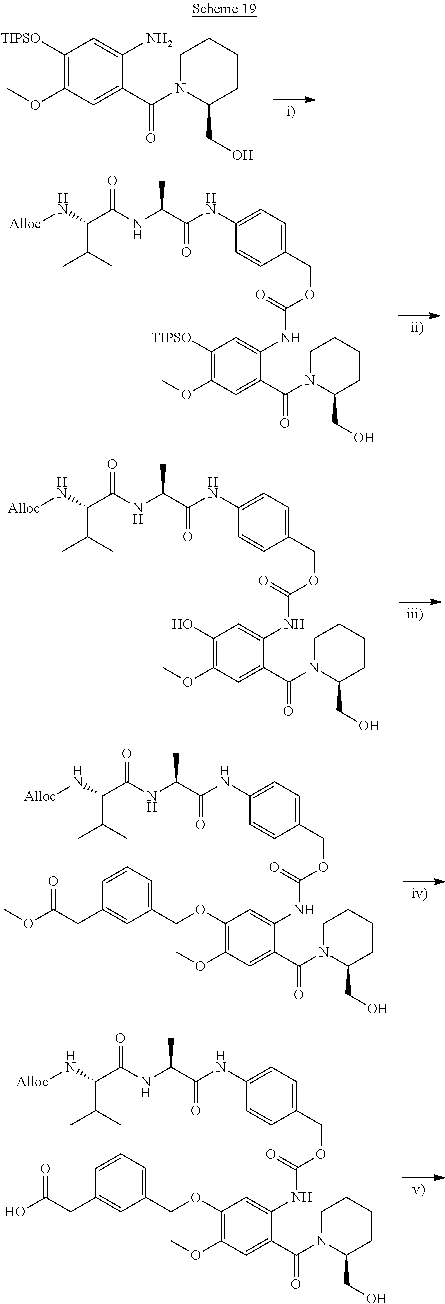

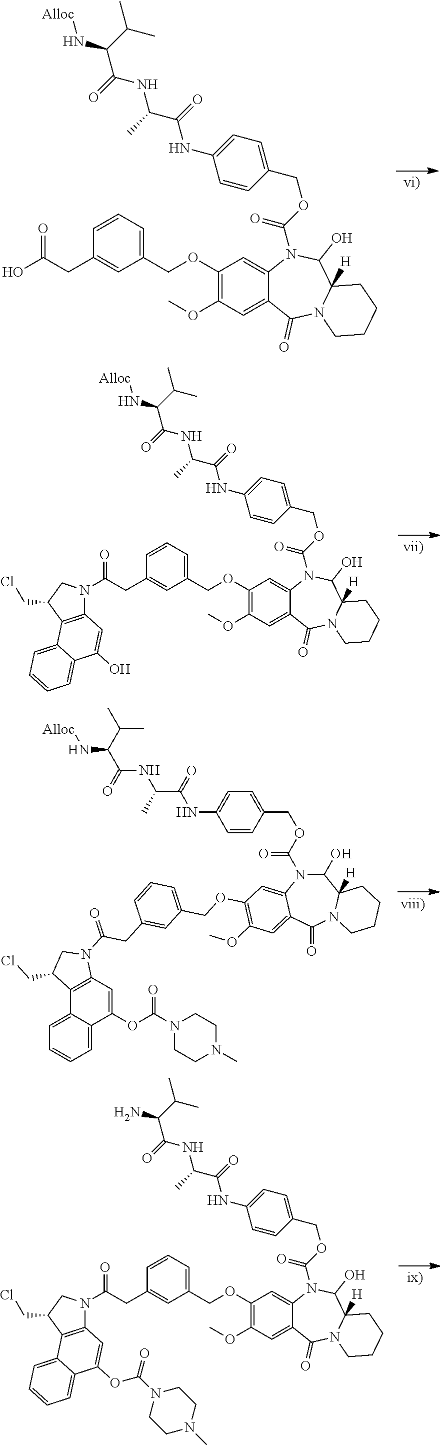

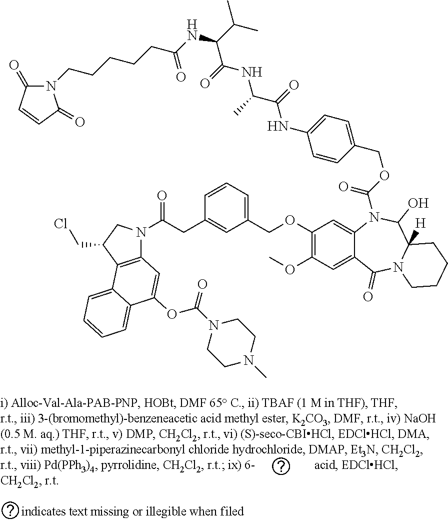 [SMILES: COc1cc2c(cc1OCc1cccc(CC(=O)N3C[C@@H](CCl)c4c3cc(OC(=O)N3CCN(C)CC3)c3ccccc43)c1)N(C(=O)OCc1ccc(NC(=O)[C@H](C)NC(=O)[C@@H](NC(=O)CCCCCN3C(=O)C=CC3=O)C(C)C)cc1)C(O)[C@@H]1CCCCN1C2=O]

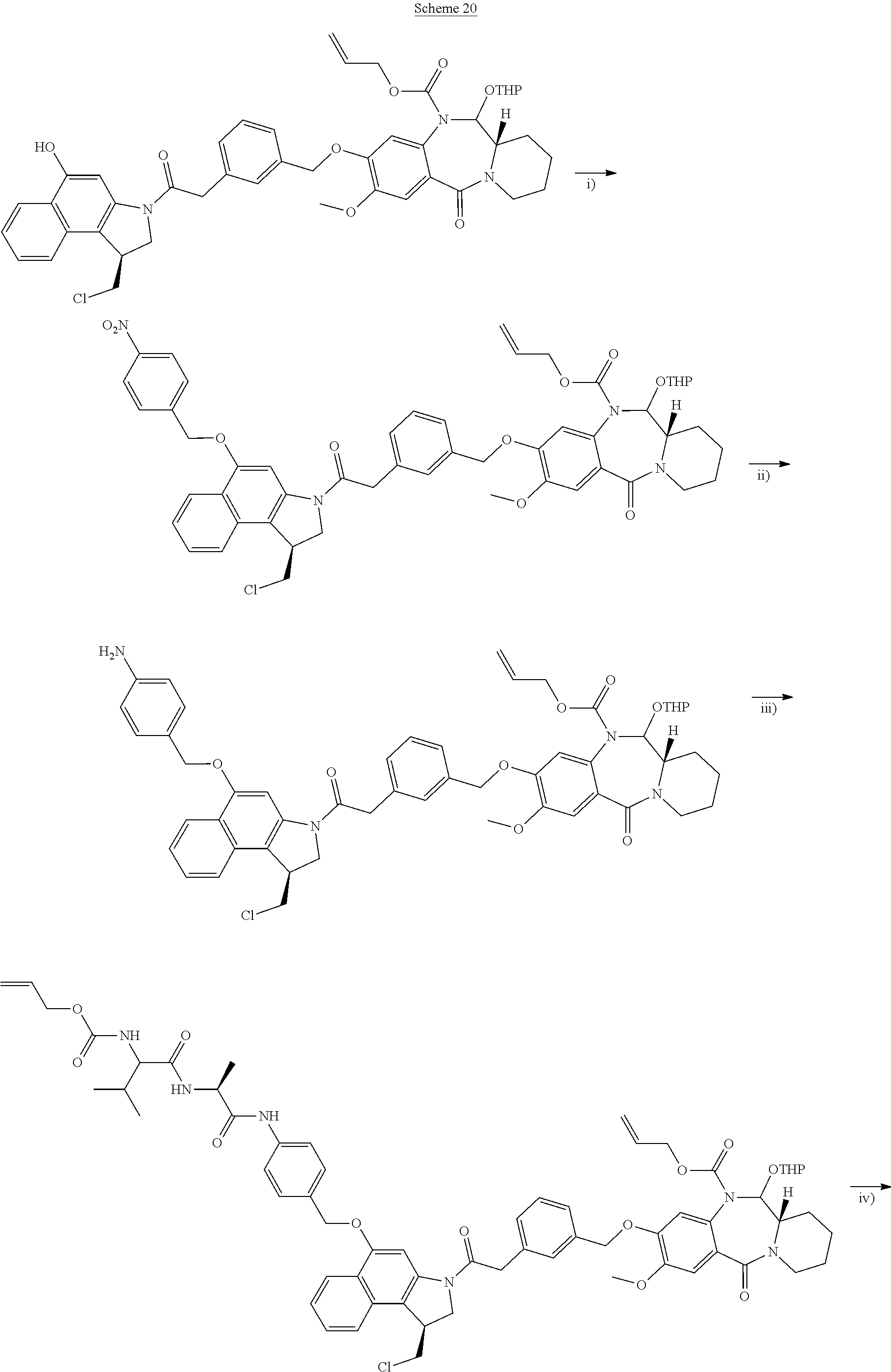

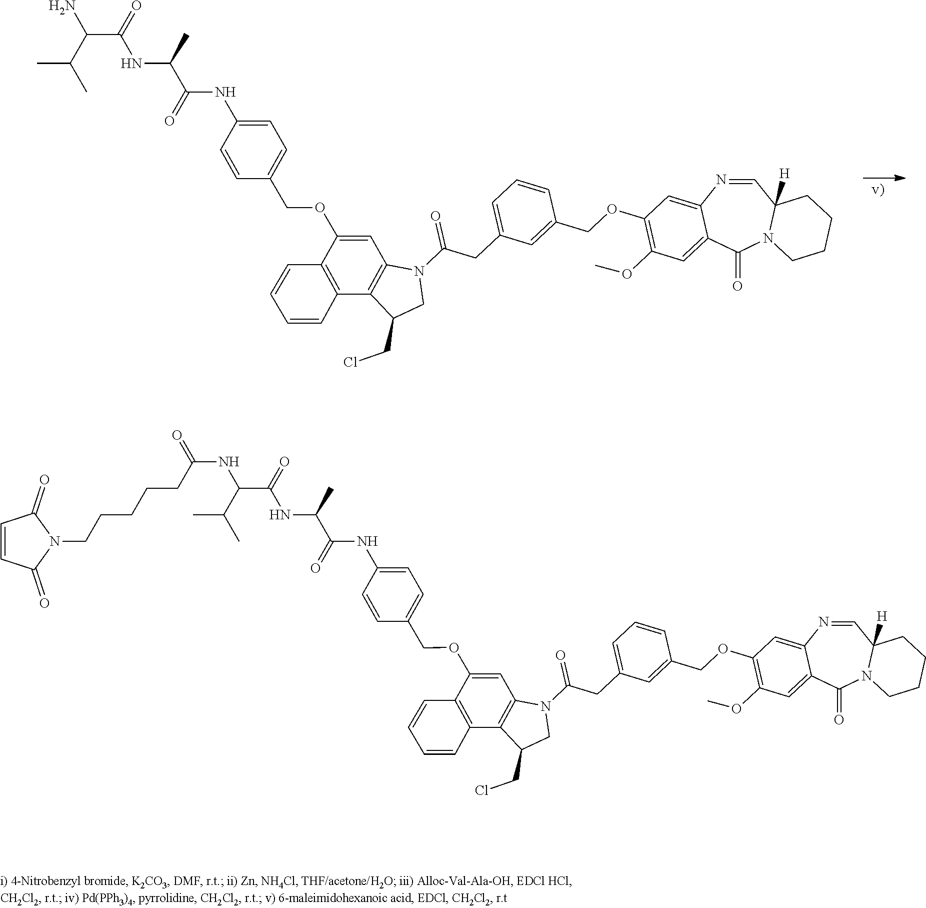 [SMILES: COc1cc2c(cc1OCc1cccc(CC(=O)N3C[C@@H](CCl)c4c3cc(OCc3ccc(NC(=O)[C@H](C)NC(=O)C(N)C(C)C)cc3)c3ccccc43)c1)N=C[C@@H]1CCCCN1C2=O ; COc1cc2c(cc1OCc1cccc(CC(=O)N3C[C@@H](CCl)c4c3cc(OCc3ccc(NC(=O)[C@H](C)NC(=O)C(NC(=O)CCCCCN5C(=O)C=CC5=O)C(C)C)cc3)c3ccccc43)c1)N=C[C@@H]1CCCCN1C2=O]

### Example 65: 4-Hydroxy-5-methoxy-2-nitrobenzaldehyde (65)

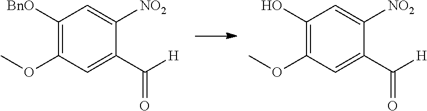 [SMILES: COc1cc(C=O)c([N+](=O)[O-])cc1OCc1ccccc1 ; COc1cc(C=O)c([N+](=O)[O-])cc1O]

A solution of 4-(benzyloxy)-5-methoxy-2-nitrobenzaldehyde (32) (100 g, 348 mmol) in glacial acetic acid (800 mL) was charged with an aqueous solution of hydrobromic acid (48% v/v, 88.0 mL, 522 mmol) and heated to 85° C., with stirring for 1 h, after which the reaction was judged to be complete by TLC. After allowing the resulting mixture to cool to room temperature, it was then diluted in water (1.60 L), and the resulting precipitate filtered, and washed with cold water (100 mL×3) to give the title compound (50.0 g, 73%) as a yellow solid, which was used immediately in the subsequent step without further purification.

¹H NMR (400 MHz, DMSO-d₆) δ 11.11 (br s, 1H), 10.15 (br s, 1H), 7.50 (s, 1H), 7.35 (s, 1H), 3.94 (s, 3H); MS (ES−): m/z=196 (M−H)⁻; LCMS (Method B): t_{R}=2.55 min.

### Example 66: 5-Methoxy-2-nitro-4-((triisopropylsilyl)oxy)benzaldehyde (66)

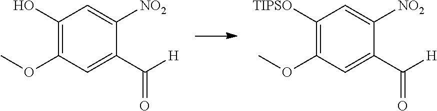

A mixture of 4-hydroxy-5-methoxy-2-nitrobenzaldehyde (65) (50.0 g, 254 mmol), triisopropylsilyl chloride (59.7 mL, 279 mmol) and imidazole (51.8 g, 761 mmol) was heated and stirred at 100° C. for 30 min. The reaction mixture was poured onto ice-water and extracted with ethyl acetate (500 mL×3). The organic extract was dried over sodium sulfate, filtered and concentrated under reduced pressure. The resulting residue was purified by flash column chromatography (silica), eluting with ethyl acetate/petroleum spirit, 40-60° C. (5%) to give the title compound (57.5 g, 64%) as a yellow solid.

¹H NMR (400 MHz, CDCl₃) δ 10.42 (s, 1H), 7.59 (s, 1H), 7.40 (s, 1H), 3.95 (s, 3H), 1.33-1.24 (m, 3H), 1.07 (s, 18H).

### Example 67: 5-Methoxy-2-nitro-4-((triisopropylsilyl)oxy)benzoic acid (67)

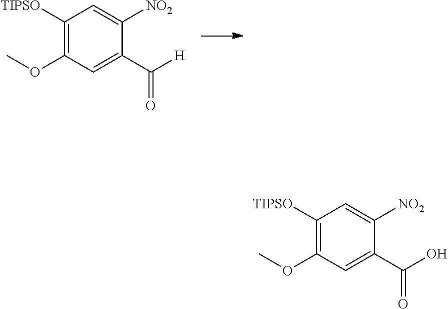

A solution of sodium chlorite (80%, 46.0 g, 407 mmol) and sodium phosphate monobasic dihydrate (35-5 g, 228 mmol) in water (200 mL) was added to a solution of 5-methoxy-2-nitro-4-((triisopropylsilyl)oxy)benzaldehyde (66) (57.5 g, 163 mmol) in tetrahydrofuran (800 mL) at room temperature. Hydrogen peroxide (30% w/w, 235 mL, 2.28 mol) was immediately added to the vigorously stirred biphasic mixture. The starting material dissolved, and the temperature of the reaction mixture rose to 45° C. After 30 min, the reaction was judged to have completed by TLC. The mixture was subsequently acidified to pH=3-4 with citric acid and extracted with ethyl acetate (500 mL×3). The combined organic extracts were washed with water (150 mL) and brine (150 mL), dried over anhydrous sodium sulfate, filtered and concentrated in vacuo. The residue was then purified by flash column chromatography (silica), eluting with ethyl acetate/petroleum spirit, 40-60° C. (10%) then methanol/dichloromethane (10%) to afford the title compound (38.0 g, 63%) as a yellow oil.

¹H NMR (400 MHz, CDCl₃) δ 9.81 (s, 1H), 7.35 (s, 1H), 7.25 (s, 1H), 3.91 (s, 3H), 1.26 (q, J=7.4 Hz, 3H), 1.09 (d, J=7.4 Hz, 18H); MS (ES−): m/z=368 (M−H)⁻; LCMS (Method D): t_{R}=4.75 min.

### Example 68: (S)-(2-(Hydroxymethyl)piperidin-1-yl)(5-methoxy-2-nitro-4((triisopropylsilyl)oxy)phenyl)methanone (68)

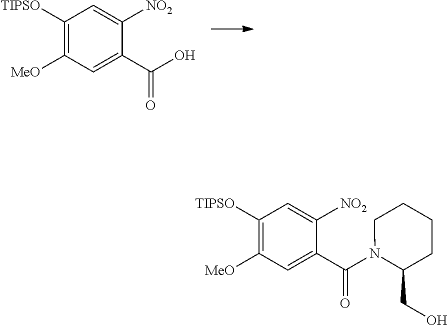

A solution of 5-methoxy-2-nitro-4-((triisopropylsilyl)oxy)benzoic acid (67) (28.0 g, 75.8 mmol), HATU (31.7 g, 83.4 mmol) and dry triethylamine (44 mL) in dry dichloromethane (300 mL) was stirred at room temperature for 30 min. (S)-Piperidin-2-ylmethanol (11.4 g, 98.5 mmol) was added and the reaction mixture was stirred at room temperature for 2 h. The reaction mixture was partitioned between dichloromethane (500 mL×2) and water (100 mL). The combined organic extracts were then dried over sodium sulfate, filtered and concentrated in vacuo. The resulting residue was purified by column chromatography (silica), eluting with ethyl acetate/petroleum spirit, 40-60° C. (from 50% to 75%), to give the title compound (20.0 g, 57%) as a yellow solid.

¹H NMR (400 MHz, CDCl₃) mixture of rotamers, δ 7.68-7.65 (m, 1H), 7.03-6.65 (m, 1H), 5.04-4.69 (m, 1H), 4.12-4.05 (m, 0.4H), 4.01-3.95 (m, 0.5H), 3.92-3.89 (m, 2.6H), 3.83-3.74 (m, 1-5H), 3.64-3.59 (m, 0.4H), 3.45-3.40 (m, 0.3H), 3.21-3.01 (m, 1.4H), 2.87-2.79 (m, 0.4H), 1.97-1.94 (m, 0.6H), 1.88-1.77 (m, 0.6H), 1.73-1.62 (m, 3H), 1.56-1.44 (m, 2H), 1.29-1.24 (m, 3H), 1.09 (d, J=7.3 Hz, 18H); MS (ES+): m/z=467 (M+H)⁺; LCMS (Method B): t_{R}=4.75 min.

### Example 69: (S)-(2-Amino-5-methoxy-4-((triisopropylsilyl)oxy)phenyl)(2-(hydroxymethyl)piperidin-1-yl)methanone (69)

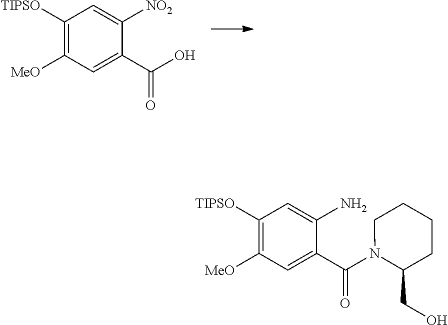

A solution of(S)-(2-(hydroxymethyl)piperidin-1-yl)(5-methoxy-2-nitro-4 ((triisopropylsilyl)oxy)phenyl)methanone (68) (1.00 g, 2.14 mmol) in tetrahydrofuran (5 mL) was charged with palladium on activated charcoal (10 wt. % basis, 100 mg), ammonium formate (1.10 g, 17.1 mmol) and water (1 mL), and stirred at room temperature, under argon, for 2 h. The resulting mixture was filtered through celite, the filter cake was washed with ethyl acetate (50 mL) and water (50 mL) and the filtrate separated. The organic phase was then extracted with brine (50 mL×2), and dried over magnesium sulfate, filtered and concentrated in vacuo. Purification by flash column chromatography (silica), eluting with ethyl acetate/petroleum spirit, 40-60° C. (from 50% to 67%), gave the title compound (892 mg, 95%) as a yellow solid.

¹H NMR (400 MHz, CDCl₃) δ 6.67 (s, 1H), 6.30 (s, 1H), 4.00-3.81 (m, 4H), 3.72 (s, 3H), 3.57 (s, 1H), 3.08 (s, 1H), 1.68-1.64 (m, 4H), 1.57-1.43 (m, 2H), 1.28-1.17 (m, 3H), 1.08 (d, J=7.4 Hz, 18H); ¹³C NMR (100 MHz, CDCl₃) δ 171.8, 147.9, 143.7, 133.2, 112.5, 110.0, 109.5, 68.7, 61.0, 56.4, 30.9, 25.8, 19.9, 17.9, 12.9; MS (ES+): m/z=437 (M+H)⁺; LCMS (Method C): t_{R}=4.07 min.

### Example 70: Allyl (S)-(2-(2-(hydroxymethyl)piperidine-1-carbonyl)-4-methoxy-5-((triisopropylsilyl)oxy)phenyl)carbamate (70)

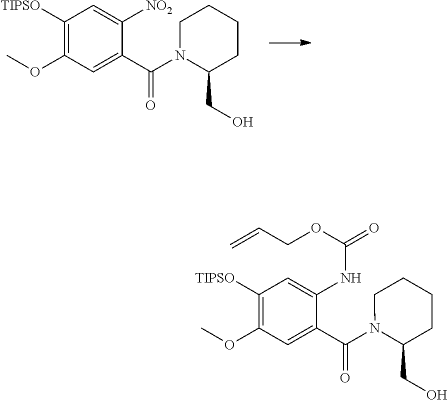

A solution of (S)-(2-amino-5-methoxy-4-((triisopropylsilyl)oxy)phenyl)(2-(hydroxylmethyl)piperidin-1-yl)methanone (69) (892 mg, 2.04 mmol) in dichloromethane (4 mL) was cooled to −10° C. and charged with pyridine (380 μL) and allyl chloroformate (228 μL, 2.14 mmol), dropwise under argon. After 35 min, the reaction mixture was diluted with dichloromethane (10 mL), then extracted with a saturated aqueous solution of copper sulfate (10 mL×2) and brine (10 mL), dried over magnesium sulfate, filtered and concentrated in vacuo. Purification by flash column chromatography (silica), eluting with ethyl acetate/petroleum spirit, 40-60° C. (from 50% to 75%), gave the title compound (907 mg, 85%) as a pale yellow solid.

¹H NMR (400 MHz, CDCl₃) δ 8.08 (s, 1H), 7.62 (s, 1H), 6.75 (s, 1H), 5.92 (ddt, J=17.2, 10.7, 55 Hz, 1H), 5.32 (dt, J=17.3, 1.7 Hz, 1H), 5.20 (dt, J=10.6, 1.4 Hz, 1H), 4.61 (dt, J=5-5, 1.5 Hz, 2H), 3.88 (t, J=10.7 Hz, 1H), 3.76 (s, 3H), 3.61-3.57 (m, 1H), 3.20-3.02 (m, 2H), 2.03 (s, 1H), 1.65-1.62 (m, 3H), 1.53-1.40 (m, 2H), 1.29-1.24 (m, 4H), 1.11-1.08 (m, 18H); MS (ES+): m/z=522 (M+H)⁺; LCMS (Method A): t_{R}=9.62 min.

### Example 71: Allyl (6aS)-6-hydroxy-2-methoxy-12-oxo-3-((triisopropyl-silyl)oxy)-6,6a,7,8,9,10-hexahydrobenzo[e]pyrido[1,2-a][1,4]diazepine-5(12H)-carboxylate (71)

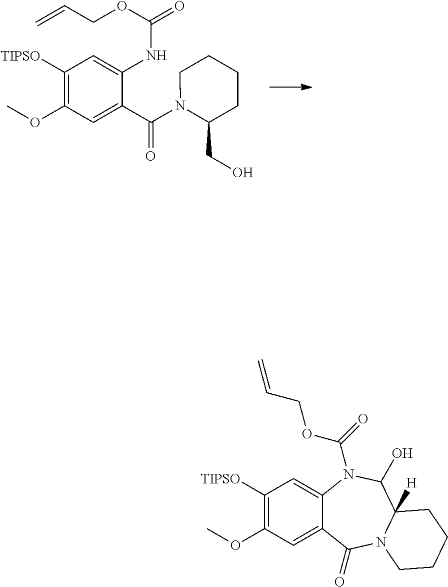

A solution of allyl (S)-(2-(2-(hydroxymethyl)piperidine-1-carbonyl)-4-methoxy-5-((triisopropylsilyl)oxy)phenyl)carbamate (70) (17.0 g, 32.7 mmol) in dichloromethane (150 mL) was charged with (diacetoxyiodo)benzene (12.6 g, 39.2 mmol) and 2,2,6,6-tetramethylpiperidine 1-oxyl (510 mg, 3.30 mmol), and stirred at room temperature for 16 h. The resulting mixture was then diluted with dichloromethane (350 mL), and sequentially washed with a saturated aqueous solution of sodium metabisulfite (100 mL) and a saturated aqueous solution of sodium hydrogen carbonate (100 mL). The organic extract was then dried over sodium sulfate, filtered and concentrated in vacuo. The resulting residue was then purified by flash column chromatography (silica), eluting with ethyl acetate/petroleum spirit, 40-60° C. (from 25% to 50%), to give the title compound (13.0 g, 77%) as a pale yellow solid.

¹H NMR (400 MHz, CDCl₃) δ 7.13 (s, 1H), 6.65 (s, 1H), 5.90 (d, J=10.3 Hz, 1H), 5.76 (s, 1H), 5.14 (t, J=12.1 Hz, 2H), 4.59 (dd, J=13.1, 5.3 Hz, 1H), 4.44 (dd, J=12.9, 5.1 Hz, 1H), 4.34 (dt, J=13.5, 4.1 Hz, 1H), 3.83 (s, 3H), 3.77 (br, 1H), 3.45 (ddd, J=10.1, 5.9, 4.0 Hz, 1H), 3.10-2.99 (m, 1H), 2.09-1.98 (m, 1H), 1.82-1.67 (m, 2H), 1.67-1.56 (m, 3H), 1.28-1.15 (m, 3H), 1.06 (dd, J=70.4, 2.5 Hz, 18H); ¹³C NMR (100 MHz, CDCl₃) δ 169.2, 156.2, 150.6, 147.6, 131.9, 127.0, 125.7, 121.2, 118.2, 110.9, 82.3, 66.9, 55.5, 55.3, 38.6, 23.2, 23.0, 18.2, 17.8, 12.8; MS (ES+): m/z=519 (M+H)⁺; LCMS (Method A): t_{R}=8.67 min.

### Example 72: Allyl (6aS)-2-methoxy-12-oxo-6-((tetrahydro-2H-pyran-2-yl)oxy)-3-((triisopropylsilyl)oxy)-6,6a,7,8,9,10-hexahydrobenzo[e]-pyrido[1,2-a][1,4]diazepine-5(12H)-carboxylate (72)

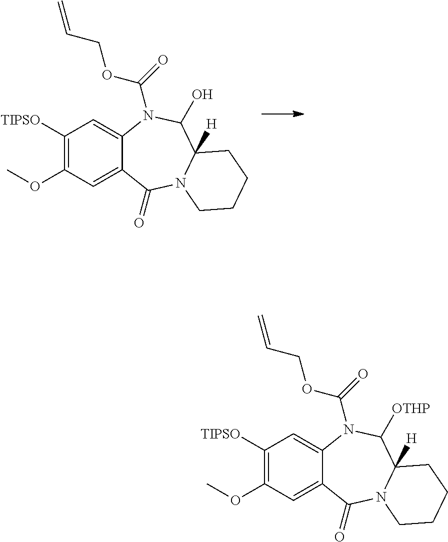

A solution of allyl (6aS)-6-hydroxy-2-methoxy-12-oxo-3-((triisopropylsilyl)oxy)-6,6a,7,8,9,10-hexahydrobenzo[e]pyrido[1,2-a][1,4]diazepine-5(12H)-carboxylate (71) (14.0 g, 27.0 mmol) in tetrahydrofuran (130 mL) was charged with 3,4-dihydro-2H-pyran (24.6 g, 270 mmol) and p-toluenesulfonic acid monohydrate (140 mg, 0.76 mmol), and stirred for 18 h at room temperature. The resulting mixture was then diluted with ethyl acetate (360 mL) and washed with a saturated aqueous solution of sodium hydrogen carbonate (200 mL) and brine (100 mL). The organic phase was dried over sodium sulfate, filtered and concentrated in vacuo. Purification by flash column chromatography (silica), eluting with ethyl acetate/petroleum spirit, 40-60° C. (17%), gave the title compound (12.5 g, 77%) as a yellow oil.

¹H NMR (400 MHz, CDCl₃), 1:1 mixture of diastereomers, δ 7.13 (s, 0.4H), 7.10 (s, 0.5H), 6.90 (s, 0.5H), 6.52 (s, 0.4H), 6.15 (d, J=10.0 Hz, 0.4H), 5.98 (d, J=10.0 Hz, 0.5H), 5.80-5.68 (m, 1H), 5.17-4.94 (m, 3H), 4.64-4.21 (m, 3H), 3.91-3.85 (m, 1H), 3.83 (d, J=1.8 Hz, 3H), 3.66-3.39 (m, 2H), 3.14-3.00 (m, 1H), 2.08-1.87 (m, 1H), 1.83-1.33 (m, 12H), 1.26-1.19 (m, 3H), 1.08-1.05 (m, 18H); MS (ES+): m/z=603 (M+H)⁺; LCMS (Method A): t_{R}=9.95 min.

### Example 73: Allyl (6aS)-3-hydroxy-2-methoxy-12-oxo-6-((tetrahydro-2H-pyran-2-yl)oxy)-6,6a,7,8,9,10-hexahydrobenzofelpyrido[1,2-a][1,4]-diazepine-5(12H)-carboxylate (3)

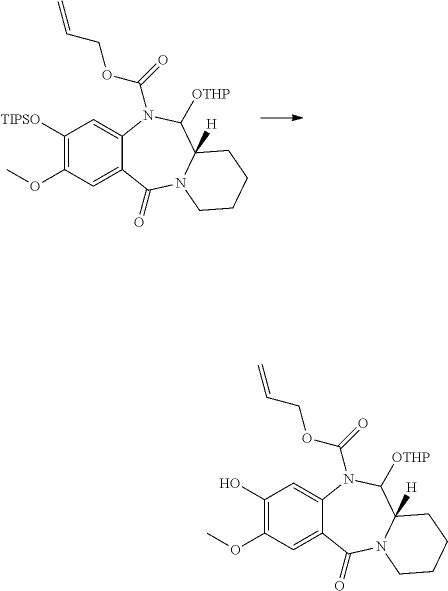

A solution of allyl (6aS)-2-methoxy-12-oxo-6-((tetrahydro-2H-pyran-2-yl)oxy)-3-((triisopropylsilyl)oxy)-6,6a,7,8,9,10-hexahydrobenzo[e]pyrido[1,2-a][1,4]diazepine-5(12H)-carboxylate (72) (10.5 g, 17.4 mmol) in tetrahydrofuran (33 mL) was charged with tetrabutylammonium fluoride (1 M in tetrahydrofuran, 26.1 mL, 26.1 mmol) at room temperature and stirred, to give an instantaneous orange colour. After 10 min, the reaction mixture was concentrated in vacuo, then immediately purified by flash column chromatography (silica), eluting with ethyl acetate/petroleum spirit, 40-60° C. (from 0% to 100%), to give the title compound (7.13 g, 92%) as a white solid.

¹H NMR (400 MHz, CDCl₃), mixture of diastereomers, δ 7.16 (s, 0.4H), 7.13 (s, 0.6H), 6.91 (br, 0.5H), 6.62 (br, 0.3H), 6.16 (d, J=10.1 Hz, 0.4H), 5.99 (d, J=10.1 Hz, 0.6H), 5.76 (ddd, J=15.8, 10.3, 5.0 Hz, 0.8H), 5.14-5.03 (m, 2H), 4.98 (br, 0.5H), 4.61 (dd, J=13.7, 4.9 Hz, 0.8H), 4.55-4.48 (m, 0.3H), 4.48 (br, 0.3H), 4.40 (dd, J=13.9, 5.2 Hz, 0.5H), 4.32-4.20 (m, 1H), 3.90 (s, 3H), 3.88-3.81 (m, 1H), 3.63 (br, 0.4H), 3.59-3.52 (m, 0.6H), 3.49-3.43 (m, 1H), 3.11-2.98 (m, 1H), 1.97-1.89 (m, 1H), 1.80-1.47 (m, 12H); ¹³C NMR (100 MHz, CDCl₃), mixture of diastereomers, δ 169.4, 169.2, 155.8, 147.9, 147.7, 146.6, 146.4, 132.2, 132.0, 128.4, 128.2, 125.9, 125.5, 117.0, 116.4, 115.8, 110.2, 109.8, 100.5, 95.3, 88.0, 84.1, 66.5, 66.3, 63.8, 63.2, 60.4, 56.2, 56.1, 55.4, 55.2, 38.8, 38.8, 31.0, 30.6, 25.2, 25.1, 23.22, 23.1, 23.0, 23.0, 20.1, 19.7, 18.4, 18.2; MS (ES+): m/z=447 (M+H)⁺; LCMS (Method A): t_{R}=6.87 min.

### Example 74: 3-(Methoxycarbonyl)-4-phenylbut-3-enoic acid (74)

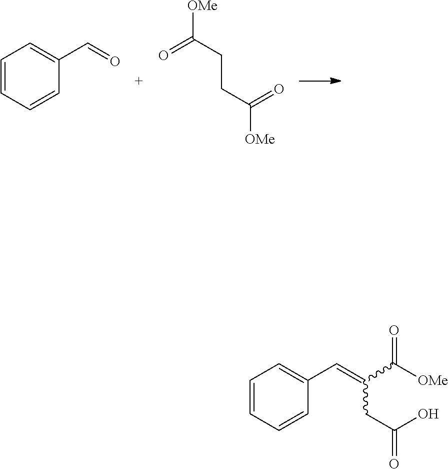

A solution of benzaldehyde (100 g, 942 mmol) and dimethyl succinate (206 g, 1.41 mol) in tert-butanol (500 mL) was added to a refluxing solution of potassium tert-butoxide (158 g, 1.41 mol) in tert-butanol (1.5 L) over 1 h. The mixture was then stirred for a further 30 min before being allowed to cool to room temperature. After concentrating in vacuo, the resulting residue was diluted with water (500 mL) and extracted with ethyl acetate (500 mL). The aqueous phase was then acidified to pH=4-5 with an aqueous solution of hydrochloric acid (6 M), then extracted with ethyl acetate (1 L). The combined organic extracts were dried over sodium sulfate, filtered and concentrated in vacuo to give the title compound (300 g, impure) as a yellow oil which was used in the subsequent step without further purification.

MS (ES+): m/z=221 (M+H)⁺; LCMS (Method F): t_{R}=3.23 min.

### Example 75: Methyl 4-hydroxy-2-naphthoate (75)

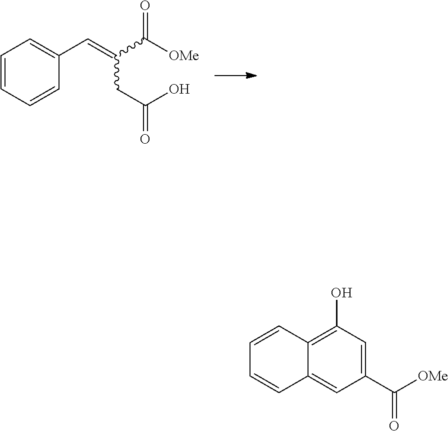

A solution of 3-(methoxycarbonyl)-4-phenylbut-3-enoic acid (74) (300 g) and trifluoroacetic anhydride (99.3 mL, 714 mmol) in tetrahydrofuran (1.5 L) was stirred at 70° C. for 5 h, after which, consumption of starting material was confirmed by TLC. The reaction mixture was then concentrated in vacuo, adjusted to pH=8-9 with an aqueous solution of sodium hydroxide (1 M) and extracted with ethyl acetate (1 L). The organic phase was then dried over sodium sulfate and concentrated in vacuo. Recrystallisation from ethyl acetate/petroleum spirit, 40-60° C. (10%) gave the title compound (100 g, 53%) as a yellow solid.

MS (ES+): m/z=202 (M+H)⁺; LCMS (Method F): t_{R}=3.55 min.

### Example 76: Methyl 4-(benzyloxy)-2-naphthoate (76)

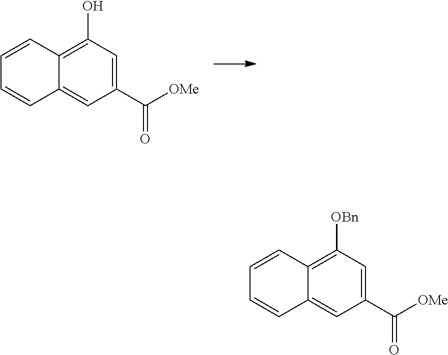 [SMILES: COC(=O)c1cc(O)c2ccccc2c1 ; COC(=O)c1cc(OCc2ccccc2)c2ccccc2c1]

A solution of methyl 4-hydroxy-2-naphthoate (75) (200 g, 990 mmol), benzyl bromide (203 g, 1.19 mol) and caesium carbonate (386 g, 1.19 mol) in N,N-dimethylformamide (800 mL) was stirred at 90° C. for 16 h, after which TLC confirmed consumption of starting material. The mixture was diluted in ethyl acetate (1.5 L), washed with water (1 L×2), then brine (500 mL), dried over sodium sulfate and concentrated in vacuo to give the title compound (250 g, 86%) as a white solid, which was used in the subsequent step without further purification.

### Example 77: 4-(Benzyloxy)-2-naphthoic acid (77)

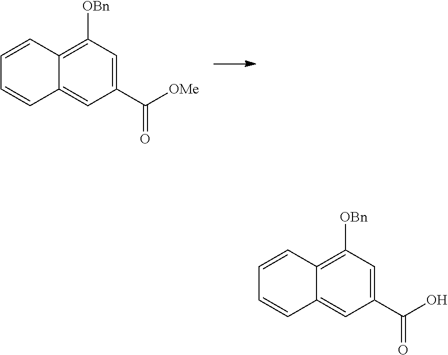 [SMILES: COC(=O)c1cc(OCc2ccccc2)c2ccccc2c1 ; O=C(O)c1cc(OCc2ccccc2)c2ccccc2c1]

A solution of methyl 4-(benzyloxy)-2-naphthoate (76) (250 g, 856 mmol) in toluene (500 mL) was charged with an aqueous solution of sodium hydroxide (12 M, 300 mL) and heated to 100° C. for 16 h, after which TLC confirmed the consumption of starting material. The organic phase was separated and concentrated in vacuo. The residue was then taken up into ethyl acetate (1.5 L) and acidified to pH=2 with an aqueous solution of hydrochloric acid (6 M). The organic phase was separated, dried over sodium sulfate and concentrated in vacuo. Recrystallization from ethyl acetate/petroleum spirit, 40-60° C. (10%) gave the title compound (90 g, 32%) as a white solid.

MS (ES+): m/z=279 (M+H)⁺; LCMS (Method F): t_{R}=4.09 min.

### Example 78: tert-Butyl (4-(benzyloxy)naphthalen-2-yl)carbamate (78)

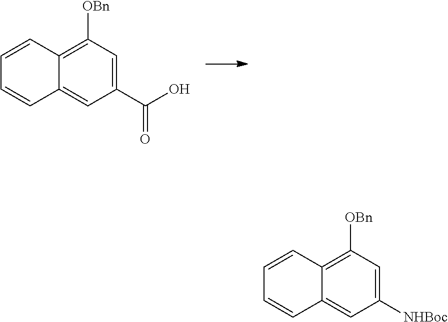 [SMILES: O=C(O)c1cc(OCc2ccccc2)c2ccccc2c1 ; CC(C)(C)OC(=O)Nc1cc(OCc2ccccc2)c2ccccc2c1]

A solution of 4-(benzyloxy)-2-naphthoic acid (77) (50.0 g, 180 mmol), diphenyl phosphoryl azide (41.5 mL, 234 mmol) and triethylamine (28.9 mL, 270 mmol) in toluene (300 mL) was stirred at room temperature for 1 h, after which TLC showed consumption of starting material. tert-Butanol (200 mL) was added and the resulting mixture was stirred at 90° C. for 17 h. This was then diluted with ethyl acetate (1.5 L) and water (500 mL). The organic phase was separated, dried over sodium sulfate, filtered and concentrated in vacuo. Recrystallization from ethyl acetate/petroleum spirit, 40-60° C. (10%) gave the title compound (35 g, 56%) as a pink solid.

MS (ES+): m/z=350 (M+H)⁺; LCMS (Method F): t_{R}=4.67 min.

### Example 79: tert-Butyl (4-(benzyloxy)-1-iodonaphthalen-2-yl)carbamate

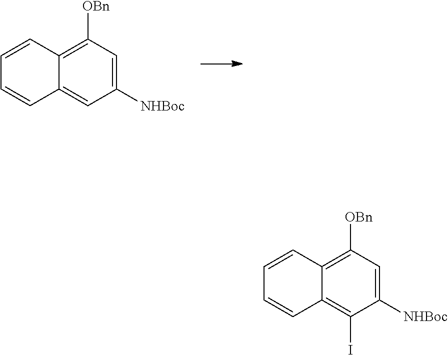 [SMILES: CC(C)(C)OC(=O)Nc1cc(OCc2ccccc2)c2ccccc2c1 ; CC(C)(C)OC(=O)Nc1cc(OCc2ccccc2)c2ccccc2c1I]

A mixture of tert-butyl (4-(benzyloxy)naphthalen-2-yl)carbamate (78) (55.0 g, 157 mmol), iodic acid (5.50 g, 31.5 mmol) and iodine (16.0 g, 63 mmol) in methanol (400 mL) and water (100 mL) was stirred at 80° C. for 5 h, after which TLC showed consumption of starting material. The mixture was diluted with water (1.0 L) and filtered. The resulting cake was washed with methanol (200 mL) and concentrated in vacuo to give the title compound (72 g, 96%) as a brown solid.

MS (ES+): m/z=476 (M+H)⁺; LCMS (Method E): t_{R}=4.91 min.

### Example 80: tert-Butyl (R)-(4-(benzyloxy)-1-iodonaphthalen-2-yl)(oxiran-2-ylmethyl)carbamate (80)

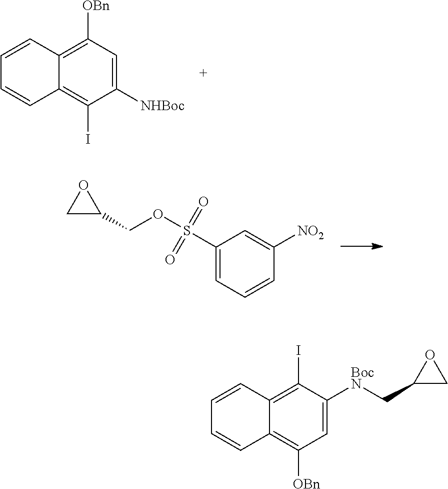

A solution of tert-butyl (4-(benzyloxy)-1-iodonaphthalen-2-yl)carbamate (79) (52 g, 109 mmol) in N,N-dimethylformamide (500 mL) was charged with sodium hydride (60% dispersion in mineral oil, 17 g, 425 mmol) and stirred at room temperature for 30 min, after which (S)-oxiran-2-ylmethyl 3-nitrobenzenesulfonate (51 g, 197 mmol) was added and the resulting mixture stirred for a further 3 h. TLC confirmed consumption of starting material. The reaction mixture was poured cautiously onto ice-water (500 mL) and extracted with ethyl acetate (1.0 L). The organic phase was separated, and washed with water (500 mL) and brine (300 mL), then dried over sodium sulfate and concentrated in vacuo to give the title compound (55 g, 95%) as a white solid.

¹H NMR (400 MHz, CDCl₃) δ 8.33-8.32 (m, 1H), 8.41-8.20 (m, 1H), 7.59-7.48 (m, 4H), 7.45-7.33 (m, 3H), 6.94-6.83 (m, 1H), 5.28 (s, 2H), 4.15-4.09 (m, 1H), 3.50-3.42 (m, 1H), 3.14-3.13 (m, 1H), 2.82-2.60 (m, 1H), 2.41 (ddd, J=12.4, 4.8, 2.8 Hz, 1H), 1.33-1.31 (m, 9H).

### Example 81: tert-Butyl (S)-5-(benzyloxy)-1-(hydroxymethyl)-1,2-dihydro-3H-benzo[e]indole-3-carboxylate (81)

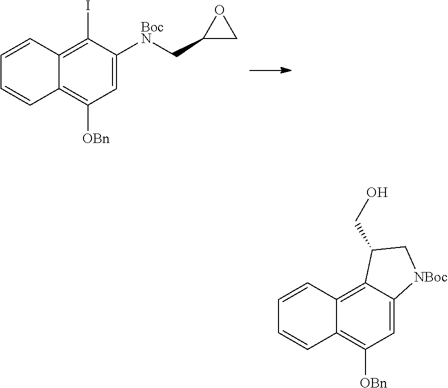 [SMILES: CC(C)(C)OC(=O)N(C[C@@H]1CO1)c1cc(OCc2ccccc2)c2ccccc2c1I ; CC(C)(C)OC(=O)N1C[C@@H](CO)c2c1cc(OCc1ccccc1)c1ccccc21]

Zinc chloride (1 M in tetrahydrofuran, 28 mL) was diluted in anhydrous tetrahydrofuran (40 mL) and cooled to 0° C., under an inert atmosphere of argon. A solution of methyl lithium (1.6 M in diethyl ether, 70.6 mL) was then added to the cooled mixture, dropwise, and stirred for 30 min, before cooling further to −78° C. (Trimethylsilyl)isothiocyanate (4 mL, 28.2 mmol) was added dropwise to the reaction mixture at −78° C., before warming to 0° C. for 30 min and then again cooling to −78° C.

A solution of tert-butyl (R)-(4-(benzyloxy)-1-iodonaphthalen-2-yl)(oxiran-2-ylmethyl)carbamate (80) (10 g, 18.8 mmol) in tetrahydrofuran (20 mL) was added dropwise to the reaction mixture at −78° C. for 30 min, then warmed to 0° C. for 1 h, followed by room temperature for 30 min. After quenching with a saturated aqueous solution of ammonium chloride, the mixture was extracted with dichloromethane (500 mL×3) and the combined organics were washed with brine (100 mL), dried over sodium sulfate and concentrated in vacuo to give the title compound (10 g, impure), which was used in the subsequent step without further purification.

¹H NMR (400 MHz, CDCl₃) δ 8.29 (d, J=8.4 Hz, 1H), 7.90 (s, 1H), 7.71 (d, J=8.2 Hz, 1H), 7.55 (d, J=6.8 Hz, 2H), 7.51-7.40 (m, 3H), 7.36-7.32 (m, 2H), 5.27 (s, 2H), 4.22 (d, J=11.4 Hz, 1H), 4.13 (t, J=10.0 Hz, 1H), 4.01-3.95 (m, 1H), 3.85 (bs, 1H), 3.81-3.73 (m, 1H), 1.60 (s, 9H); MS (ES+): m/z=406 (M+H)⁺; LCMS (Method F): t_{R}=4.69 min.

### Example 82: tert-Butyl (S)-5-(benzyloxy)-1-(chloromethyl)-1,2-dihydro-3H-benzo[e]indole-3-carboxylate (82)

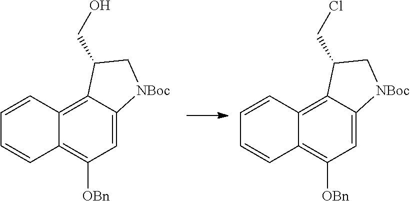 [SMILES: CC(C)(C)OC(=O)N1C[C@@H](CO)c2c1cc(OCc1ccccc1)c1ccccc21 ; CC(C)(C)OC(=O)N1C[C@@H](CCl)c2c1cc(OCc1ccccc1)c1ccccc21]

A solution of tert-butyl (S)-5-(benzyloxy)-1-(hydroxymethyl)-1,2-dihydro-3H-benzo[e]indole-3-carboxylate (81) (10.0 g, 12.4 mmol), carbon tetrachloride (30 mL) and triphenylphosphine (3.90 g, 14.8 mmol) in dichloromethane (50 mL) was stirred at room temperature for 2 h, after which, TLC showed consumption of starting material.

The reaction mixture was then concentrated in vacuo. Purification by flash column chromotography (silica), eluting with ethyl acetate/petroleum spirit, 40-60° C. (10%), followed by recrystallisation from dichloromethane/petroleum spirit, 40-60° C. (90%) gave the title compound (1.47 g, 28%) as a white solid.

[α]D²³=−14.5° (c 0.470, CH₂Cl₂); ¹H NMR (400 MHz, CDCl₃) δ 8.29 (d, J=8.4 Hz, 1H), 7.86 (s, 1H), 7.65 (d, J=8.4 Hz, 1H), 7.58-7.30 (m, 7H), 5.27 (s, 2H), 4.27-4.24 (m, 1H), 4.13 (t, J=10.6 Hz, 1H), 4.01-3.87 (m, 2H), 3.44 (t, J=10.4 Hz, 1H), 1.61 (s, 9H); MS (ES+): m/z=424 (M+H)⁺; LCMS (Method D): t_{R}=4.27 min.

### Example 83: (S)-1-(Chloromethyl)-2,3-dihydro-1H-benzo[e]indol-5-ol hydrochloride (11)

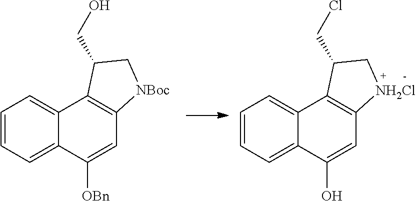

A solution of tert-butyl (S)-5-(benzyloxy)-1-(chloromethyl)-1,2-dihydro-3H-benzo[e]indole-3-carboxylate (82) (100 mg, 0.236 mmol) in anhydrous dichloromethane (3 mL) was charged with boron trichloride (1 M solution in dichloromethane, 708 μL, 0.708 mmol), in a dropwise manner via syringe, at room temperature and stirred under an inert atmosphere of argon. The resulting orange solution was stirred for 5 min before being quenched by cautious addition of methanol (5 mL), then concentrated in vacuo. The residue was charged again with methanol (5 mL) and re-concentrated in vacuo. Diethyl ether (5 mL) was then charged and the residue concentrated in vacuo once again. The residue was then subjected to high vacuum for 30 min to give the title compound (55 mg, impure) as a pale green crystalline solid (unstable), which was used immediately in the subsequent step (amide coupling) without further purification.

MS (ES+): m/z=234 (M+H)⁺; LCMS (Method C): t_{R}=2.62 min.

### Example 84: tert-Butyl (8bR,9aS)-4-oxo-9,9a-dihydro-1H-benzo[e]cyclopropa[c]indole-2(4H)-carboxylate (83)

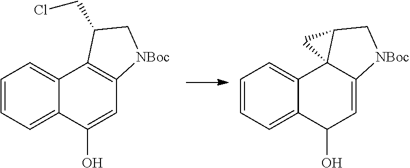 [SMILES: CC(C)(C)OC(=O)N1C[C@@H](CCl)c2c1cc(O)c1ccccc21 ; CC(C)(C)OC(=O)N1C[C@H]2C[C@@]23C1=CC(O)c1ccccc13]

A solution of tert-butyl (S)-1-(chloromethyl)-5-hydroxy-1,2-dihydro-3H-benzo[e]indole-3-carboxylate (10) (20 mg, 0.060 mmol) in anhydrous N,N-dimethylacetamide (1.0 mL) was cooled to 0° C. and charged with potassium carbonate (58.0 mg, 0.419 mmol) and stirred at this temperature for 25 min. The reaction mixture was then quenched (cold) with a saturated aqueous solution of sodium hydrogen carbonate and the resulting slurry extracted twice with ethyl acetate. The combined organic extracts were then dried over magnesium sulfate and concentrated in vacuo before purification was enacted by flash column chromatography (silica), eluting with ethyl acetate/petroleum spirit, 40-60° C. (25%, isocratic) to give the title compound (14 mg, 79%) as a yellow solid.

¹H NMR (400 MHz, CDCl₃) δ 8.22 (dd, J=70.9, 1.1 Hz, 1H), 7.49 (dd, J=70.7, 1.4 Hz, 1H), 7.39 (dt, J=70.6, 1.2 Hz, 1H), 6.86 (dd, J=70.8, 0.6 Hz, 1H), 6.82 (br s, 1H), 4.04-3.96 (m, 2H), 2.79-2.73 (m, 1H), 1.62 (dd, J=70.7, 4.4 Hz, 1H), 1.57 (s, 9H), 1.47 (t, J=4.7 Hz, 1H); ¹³C NMR (100 MHz, CDCl₃) δ 186.1, 159.7, 151.7, 140.2, 132.7, 131.8, 126.9, 126.5, 120.9, 108.7, 83.5, 52.9, 33.5, 28.2, 23.4, 14.1; MS (ES+): m/z=298 (M+H)⁺; LCMS (Method C): t_{R}=3.37 min.

### Example 85: tert-Butyl (S)-1-(chloromethyl)-5-((4-methylpiperazine-1-carbonyl)oxy)-1,2-dihydro-3H-benzo[e]indole-3-carboxylate (84)

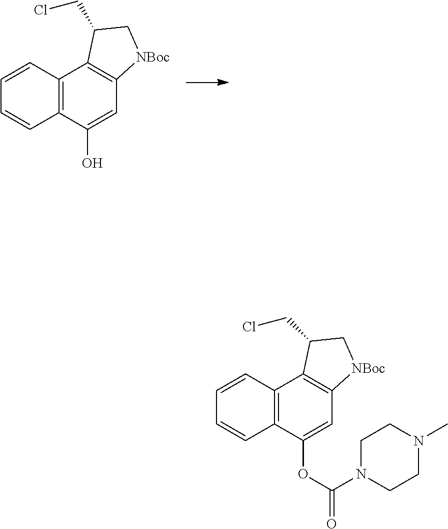 [SMILES: CC(C)(C)OC(=O)N1C[C@@H](CCl)c2c1cc(O)c1ccccc21 ; CN1CCN(C(=O)Oc2cc3c(c4ccccc24)[C@H](CCl)CN3C(=O)OC(C)(C)C)CC1]

A solution of tert-butyl (S)-1-(chloromethyl)-5-hydroxy-1,2-dihydro-3H-benzo[e]indole-3-carboxylate (to) (50 mg, 0.15 mmol) in dichloromethane (5 mL) was charged with 4-methyl-1-piperazinecarbonyl chloride hydrochloride (89 mg, 0.45 mmol), 4-(dimethylamino)pyridine (20 mg, 0.17 mmol) and triethylamine (73 μL, 0.52 mmol) and stirred at room temperature for 18 h. The reaction mixture was subsequently washed with water (2×10 mL), dried over magnesium sulfate and concentrated in vacuo, to give the title compound (59 mg, 86%) as a yellow solid.

¹H NMR (400 MHz, CDCl₃) δ 8.05 (br s, 1H), 7.82 (d, J=8.4 Hz, 1H), 7.68 (d, J=8.3 Hz, 1H), 7.51-7.45 (m, 1H), 7.38-7.33 (m, 1H), 4.28-4.21 (br, 1H), 4.15-4.07 (m, 1H), 4.03-3.96 (m, 1H), 3.94-3.88 (m, 1H), 3.72 (t, J=4.9 Hz, 2H), 3.68-3.60 (m, 2H), 3.45 (t, J=10.8 Hz, 1H), 2.61-2.50 (m, 4H), 2.31 (s, 3H), 1.57 (s, 9H); ¹³C NMR (100 MHz, CDCl₃) δ 153.4, 152.4, 148.4, 148.3, 130.2, 127.6, 124.2, 124.1, 122.6, 122.3, 120.1, 109.3, 81.2, 54.6, 54.2, 48.5, 46.3, 46.1, 45.8, 28.4; MS (ES+): m/z=460 (M+H)⁺; LCMS (Method C): t_{R}=3.00 min.

General Procedure A: O-Alkylation of pyridinobenzodiazepines

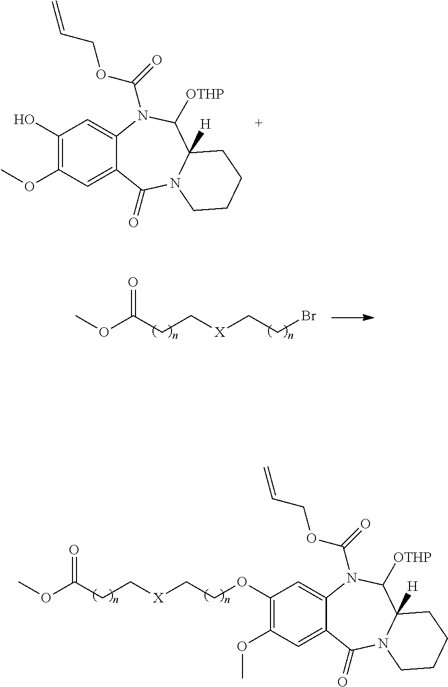

A solution of allyl (6aS)-3-hydroxy-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (73) (1 equiv.) in N,N-dimethylformamide (0.1 M) was charged with potassium carbonate (1.5 equiv.) and organohalide (1.2 equiv.) and stirred at room temperature, whilst monitoring by TLC and LCMS. Once the reaction was judged to be complete, it was diluted into ethyl acetate and washed twice with cold brine. The organic phase was dried over magnesium sulfate, filtered and concentrated in vacuo, and then purified by flash column chromatography (silica).

General Procedure B: Hydrolysis of Pyridinobenzodiazepine Methyl Esters

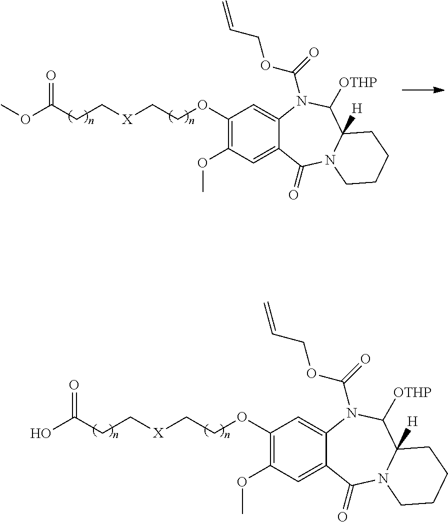

A solution of methyl ester (1 equiv.) in tetrahydrofuran (0.1 M) was charged with an aqueous solution of sodium hydroxide (0.5 M, 4 equiv.) and stirred at room temperature, whilst monitoring by TLC and LCMS. Once the reaction was judged to be complete, the mixture was then partially concentrated in vacuo (to remove tetrahydrofuran), then diluted into ethyl acetate and acidified to pH=3-4 with a saturated aqueous solution of citric acid. The organic layer was separated, and the aqueous layer washed with ethyl acetate. The combined organic extracts were then washed with brine, dried over magnesium sulfate, filtered and concentrated in vacuo. The resulting residue was used in the next step without any further purification.

General Procedure C: Amide Coupling of (S)-seco-CBI to Pyridinobenzodiazepine Carboxylic Acids

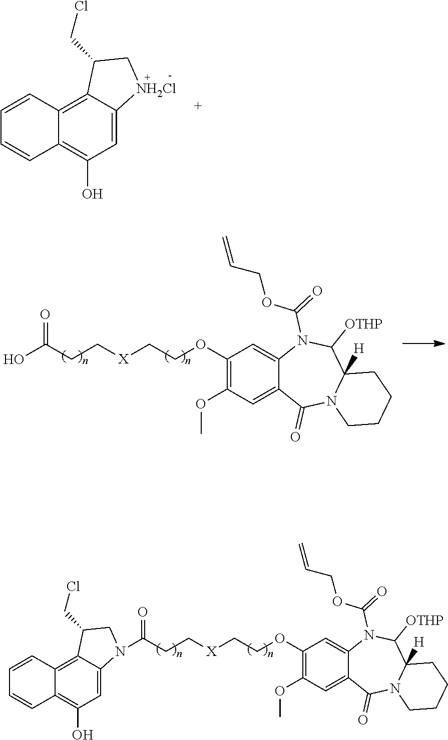

A solution of pyridinobenzodiazepine carboxylic acid (1 equiv.) in N,N-dimethylacetamide (0.1 M) was charged to (S)-1-(chloromethyl)-2,3-dihydro-1H-benzo[e]indol-5-ol hydrochloride (11) (1.4 equiv.), followed by N-(3-dimethylaminopropyl)-N′-ethylcarbodiimide hydrochloride (4 equiv.) and stirred at room temperature for 16 h. The resulting mixture was diluted into ethyl acetate and washed with cold brine (twice), then dried over magnesium sulfate, filtered and concentrated in vacuo. Purification was enacted by flash column chromatography (silica).

General Procedure D: Deprotection of N-Alloc, O-THP Pyridinobenzodiazepines

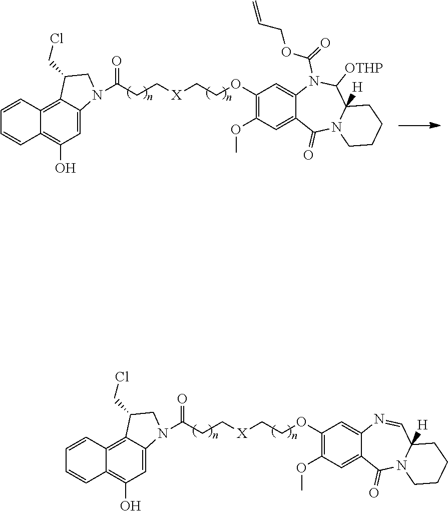

A solution of protected pyridinobenzodiazepine (1 equiv.) in dichloromethane (0.1 M) was charged with pyrrolidine (1.2 equiv.), and tetrakis(triphenylphosphine) palladium(0) (0.1 equiv.) and stirred at room temperature whilst monitoring by TLC and LCMS. After the reaction was judged to be complete (approx. 10 min), the mixture was diluted in dichloromethane and filtered through a pad of celite. The filtrate was concentrated in vacuo, then charged with diethyl ether and concentrated again. Diethyl ether was charged once more, and the residue concentrated in vacuo for a third time. Purification was enacted by flash column chromatography (silica).

### Example 86: Allyl (6aS)-2-methoxy-3-((3-(2-methoxy-2-oxoethyl)benzyl)-oxy)-12-oxo-6-((tetrahydro-2H-pyran-2-yl)oxy)-6,6a,7,8,9,10-hexahydro-benzo[e]pyrido[1,2-a][1,4]diazepine-5(12H)-carboxylate (85)

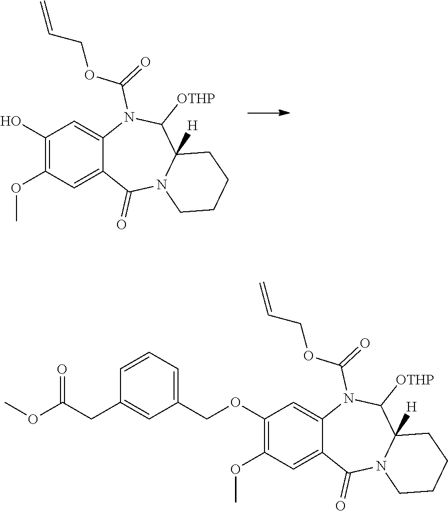

General Procedure A was followed, using allyl (6aS)-3-hydroxy-2-methoxy-12-oxo-6-((tetrahydro-2H-pyran-2-yl)oxy)-6,6a,7,8,9,10-hexahydrobenzo[e]pyrido[1,2-a][1,4]-diazepine-5(12H)-carboxylate (73) (1.00 g, 2.24 mmol), 3-(bromomethyl)-benzeneacetic acid methyl ester (653 mg, 2.69 mmol), potassium carbonate (464 mg, 3.36 mmol) and N,N-dimethylformamide (5 mL). Chromatography, eluting with ethyl acetate/petroleum spirit, 40-60° C. (66%) gave the title compound (980 mg, 74%) as a colourless gel.

¹H NMR (400 MHz, CDCl₃), mixture of diastereomers, δ 7.34-7.31 (m, 2H), 7.24-7.21 (m, 1H), 7.19 (s, 1H), 7.16 (s, 1H), 6.89 (s, 1H), 6.53 (br, 1H), 6.14 (d, J=10.4 Hz, 1H), 5.99 (d, J=10.0 Hz, 1H), 5.74-5.61 (m, 1H), 5.19-4.98 (m, 4H), 4.54 (dd, J=13.5, 4.9 Hz, 1H), 4.40-4.20 (m, 1H), 3.92-3.78 (m, 4H), 3.67 (s, 3H), 3.61 (s, 3H), 3.46 (dt, J=9.8, 4.9 Hz, 1H), 3.11-2.98 (m, 1H), 1.84-1.41 (m, 12H); ¹³C NMR (100 MHz, CDCl₃), mixture of diastereomers, δ171.8, 171.7, 169.3, 169.1, 149.9, 149.6, 149.3, 136.8, 136.7, 134.4, 134.3, 132.2, 132.0, 129.0, 128.8, 128.1, 128.0, 127.7, 126.1, 126.0, 116.9, 115.1, 114.8, 110.8, 110.4, 100.3, 95.2, 88.1, 84.2, 71.2, 70.8, 66.4, 66.2, 63.9, 63.1, 56.1, 56.1, 55.4, 55.2, 52.0, 41.1, 41.0, 38.8, 38.8, 31.0, 30.5, 25.2, 25.2, 23.2, 23.0, 22.9, 20.0, 19.6, 18.4, 18.2; MS (ES+): m/z=609 (M+H)⁺; LCMS (Method A): t_{R}=7.83 min.

### Example 87: 2-(3-((((6aS)-5-((Allyloxy)carbonyl)-2-methoxy-12-oxo-6-((tetrahydro-2H-pyran-2-yl)oxy)-5,6,6a,7,8,9,10,12-octahydrobenzo-[e]-pyrido[1,2-a][1,4]diazepin-3-yl)oxy)methyl)phenyl)acetic acid (86)

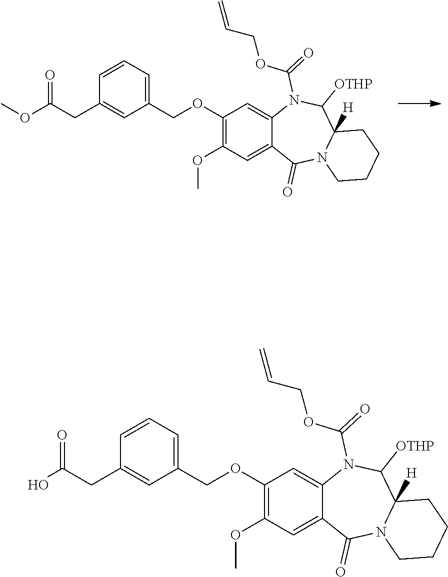

General Procedure B was followed, using allyl (6aS)-2-methoxy-3-((3-(2-methoxy-2-oxoethyl)benzyl)oxy)-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (85) (980 mg, 1.61 mmol), sodium hydroxide (0.5 M, aq., 6.4 mL, 3.22 mmol) and tetrahydrofuran (3.2 mL). The title compound (843 mg, 88%) was isolated as a white solid, which was used in the next step without further purification.

MS (ES+): m/z=595 (M+H)⁺; LCMS (Method A): t_{R}=7.12 min.

### Example 88: Allyl (6aS)-3-((3-(2-((S)-1-(chloromethyl)-5-hydroxy-1,2-dihydro-3H-benzo[e]indol-3-yl)-2-oxoethyl)benzyl)oxy)-2-methoxy-12-oxo-6-((tetrahydro-2H-pyran-2-yl)oxy)-6,6a,7,8,9,10-hexahydrobenzo[e]-pyrido[1,2-a][1,4]diazepine-5(12H)-carboxylate (87)

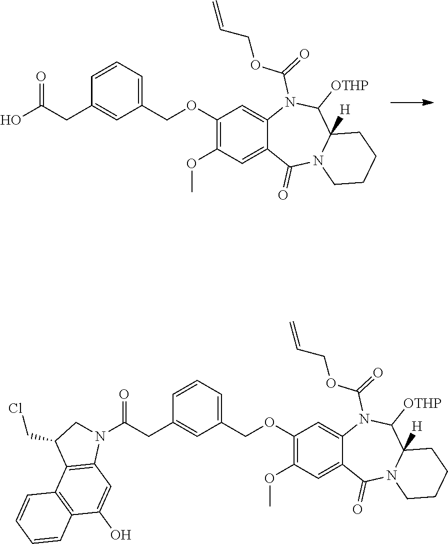

General Procedure C was followed using 2-(3-((((6aS)-5-((allyloxy)carbonyl)-2-methoxy-12-oxo-6-((tetrahydro-2H-pyran-2-yl)oxy)-5,6,6a,7,8,9,10,12-octahydrobenzo[e]pyrido[1,2-a][1,4]diazepin-3-yl)oxy)methyl)phenyl)acetic acid (29) (843 mg, 1.42 mmol), (S)-1-(Chloromethyl)-2,3-dihydro-1H-benzo[e]indol-5-ol hydrochloride (86) (536 mg, 1.98 mmol), N-(3-dimethylaminopropyl)-N′-ethylcarbodiimide hydrochloride (1.14 g, 5.95 mmol) and N,N-dimethylacetamide (2.8 mL). Chromatography, eluting with ethyl acetate/petroleum spirit, 40-60° C. (67%) gave the title compound (926 mg, 81%) as a green oil.

¹H NMR (400 MHz, CDCl₃), mixture of diastereomers, δ10.01 (br s, 1H), 8.34 (d, J=16.2 Hz, 1H), 8.23 (dd, J=8.2, 3.3 Hz, 1H), 7.60 (d, J=8.3 Hz, 1H), 7.46 (dd, J=17.7, 7.3 Hz, 2H), 7.38-7.29 (m, 4H), 7.17-7.11 (m, 1H), 6.86 (s, 0.5H), 6.56 (s, 0.5H), 6.14 (d, J=90.6 Hz, 1H), 5.97 (d, J=10.0 Hz, 1H), 5.58 (dd, J=16.4, 11.2 Hz, 1H), 5.17-4.90 (m, 5H), 4.53-4.42 (m, 0.5H), 4.39 (br, 0.5H), 4.29 (d, J=10.8 Hz, 2H), 3.96-3.89 (m, 3H), 3.82 (s, 3H), 3.79 (s, 2H), 3.60 (br, 1H) 3.52-3.36 (m, 2H), 3.27 (q, J=10.7 Hz, 1H), 3.12-2.97 (m, 1H), 1.84-1.36 (m, 12H); MS (ES+): m/z=810 (M+H)⁺; LCMS (Method A): t_{R}=8.67 min.

### Example 89: (S)-3-((3-(2-((S)-1-(Chloromethyl)-5-hydroxy-1,2-dihydro-3H-benzo[e]indol-3-yl)-2-oxoethyl)benzyl)oxy)-2-methoxy-7,8,9,10-tetrahydrobenzo[e]pyrido[1,2-a][1,4]diazepin-12(6aH)-one (42)

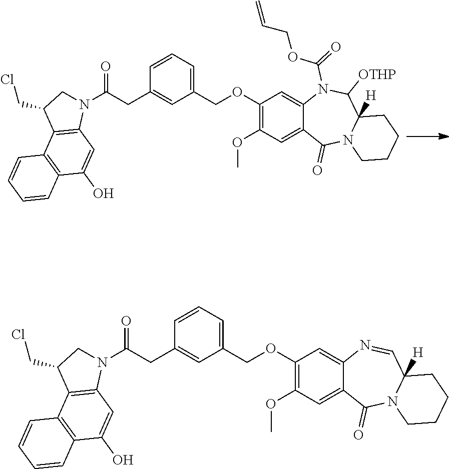

General Procedure D was followed, using allyl (6aS)-3-((3-(2-((S)-1-(chloromethyl)-5-hydroxy-1,2-dihydro-3H-benzo [e]indol-3-yl)-2-oxoethyl)benzyl)oxy)-2-methoxy-12-oxo-6-((tetrahydro-2H-pyran-2-yl)oxy)-6,6a,7,8,9,10-hexahydrobenzo [e]pyrido[1,2-a][1,4]diazepine-5(12H)-carboxylate (87) (96 mg, 0.12 mmol), tetrakis(triphenylphosphine)palladium(0) (14 mg, 0.012 mmol), pyrrolidine (12 μL, 0.14 mmol) and dichloromethane (5 mL). Chromatography, eluting with ethyl acetate/petroleum spirit, 40-60° C. (from 50% to 100%), followed by trituration from dichloromethane/diethyl ether, gave the title compound (30 mg, 41%) as a white solid.

¹H NMR (400 MHz, acetone-d₆) δ 9.34 (br s, 1H), 8.21 (d, J=8.6 Hz, 1H), 8.10 (br s, 1H), 7.92 (d, J=5.9 Hz, 1H), 7.80 (d, J=70.8 Hz, 1H), 7.75-7.67 (m, 1H), 7.65-7.59 (m, 1H), 7.57-7.50 (m, 2H), 7.40 (d, J=7.0 Hz, 1H), 7.38-7.34 (m, 2H), 6.84 (br, 1H), 5.26-5.12 (m, 1H), 5.13-5.06 (m, 1H), 4.45-4.39 (m, 2H), 4.37-4.31 (m, 2H), 4.14-4.09 (m, 2H), 4.00-3.93 (m, 2H), 3.82 (s, 3H), 3.73-3.70 (s, 1H), 3.67-3.60 (m, 1H), 2.50 (t, J=7.4 Hz, 1H), 2.32 (dt, J=70.4, 2.0 Hz, 1H), 1.82-1.77 (m, 2H), 1.64-1.55 (br, 2H); ¹³C NMR (100 MHz, acetone-d₆) δ 169.1, 166.8, 163.9, 150.5, 148.0, 140.2, 135.4, 131.8, 129.1, 128.7, 128.6, 128.5, 126.0, 123.3, 122.9, 122.4, 121.5, 114.6, 111.8, 110.6, 100.3, 70.2, 55.4, 53.2, 49.6, 46.8, 42.7, 41.7, 39.2, 29.7, 24.1, 22.9, 18.1; MS (ES+): m/z=624 (M+H)⁺; LCMS (Method C): t_{R}=4.02 min, LCMS (Method A): t_{R}=7.60 min; HRMS calculated for [C₃₆H₃₅ClN₃O₅]⁺: 624.2260, found: 624.2254;

### Example 90: Allyl (6aS)-2-methoxy-3-((2-(methoxycarbonyl)benzo[b]-thiophen-3-yl)methoxy)-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (88)

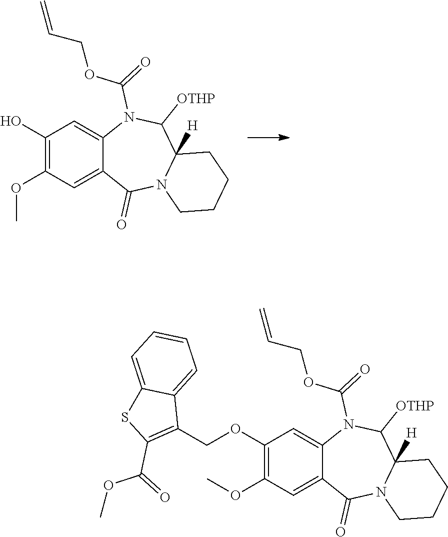

General Procedure A was followed using allyl (6aS)-3-hydroxy-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (73) (1.00 g, 2.24 mmol), methyl 3-(bromomethyl)-1-benzothiophene-2-carboxylate (766 mg, 2.69 mmol), potassium carbonate (464 mg, 3.36 mmol) and N,N-dimethylformamide (4.5 mL).

Chromatography, eluting with ethyl acetate/petroleum spirit, 40-60° C. (64%) gave the title compound (615 mg, 84%) as a white solid.

¹H NMR (400 MHz, CDCl₃), mixture of diastereomers, δ8.26 (d, J=7.9 Hz, 0.4H), 8.20 (d, J=70.8 Hz, 0.6H), 7.81 (dd, J=70.6, 5.3 Hz, 1H), 7.50-7.37 (m, 2H), 7.15 (d, J=2.9 Hz, 1H), 7.09 (s, 0.6H), 6.87 (s, 0.4H), 6.15 (d, J=10.1 Hz, 0.4H), 6.01 (d, J=9.9 Hz, 0.6H), 5.93 (d, J=12.8 Hz, 0.4H), 5.85-5.61 (m, 2.4H), 5.16-4.92 (m, 3H), 4.57 (d, J=13.7 Hz, 0.6H), 4.44 (dd, J=14.4, 9.9 Hz, 1H), 4.35-4.20 (m, 1H), 4.00-3.80 (m, 7.6H), 3.68-3.56 (m, 1H), 3.54-3.41 (m, 1H), 3.05 (t, J=11.9 Hz, 1H), 1.81-1.46 (m, 12H); ¹³C NMR (100 MHz, CDCl₃), mixture of diastereomers, δ169.3, 169.1, 163.2, 162.9, 155.8, 149.6, 140.4, 140.3, 139.1, 127.5, 127.3, 126.8, 125.4, 125.3, 125.0, 124.9, 122.4, 117.0, 115.6, 114.9, 110.5, 99.9, 95.2, 87.9, 84.0, 66.4, 66.3, 63.6, 63.4, 63.1, 62.1, 60.3, 56.1, 56.0, 55.4, 55.3, 52.5, 52.4, 38.8, 30.9, 30.6, 25.3, 25.2, 23.3, 23.2, 23.0, 22.9, 19.8, 19.7, 18.4, 18.2; MS (ES+): m/z=651 (M+H)⁺; LCMS (Method A): t_{R}=8.80 min and t_{R}=8.98.

### Example 91: 3-((((6aS)-5-((Allyloxy)carbonyl)-2-methoxy-12-oxo-6-((tetrahydro-2H-pyran-2-yl)oxy)-5,6,6a,7,8,9,10,12-octahydrobenzo[e]-pyrido[1,2-a][1,4]diazepin-1-yl)oxy)methyl)benzo[b]thiophene-2-carboxylic acid (89)

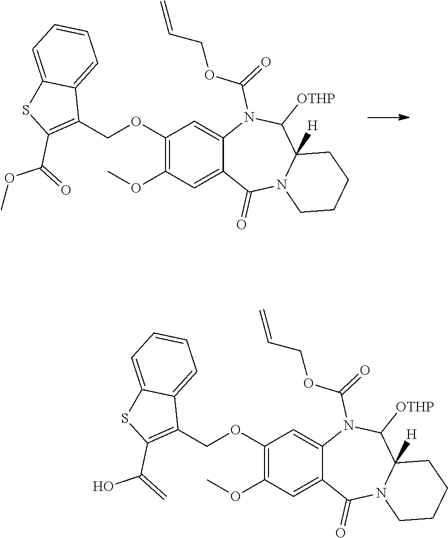

General Procedure B was followed, using allyl (6aS)-2-methoxy-3-((2-(methoxycarbonyl)benzo[b]thiophen-3-yl)methoxy)-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (88) (559 mg, 0.859 mmol), sodium hydroxide (0.5 M, aq., 3.4 mL, 1.72 mmol) and tetrahydrofuran (1.7 mL). The title compound (559 mg, impure) was isolated as a cream solid, which was used in the next step without further purification.

MS (ES+): m/z=637 (M+H)⁺; LCMS (Method A): t_{R}=7.68 min.

### Example 92: Allyl (6aS)-3-((2-((S)-1-(chloromethyl)-5-hydroxy-2,3-dihydro-1H-benzo[e]indole-3-carbonyl)benzo[b]thiophen-3-yl)methoxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (go)

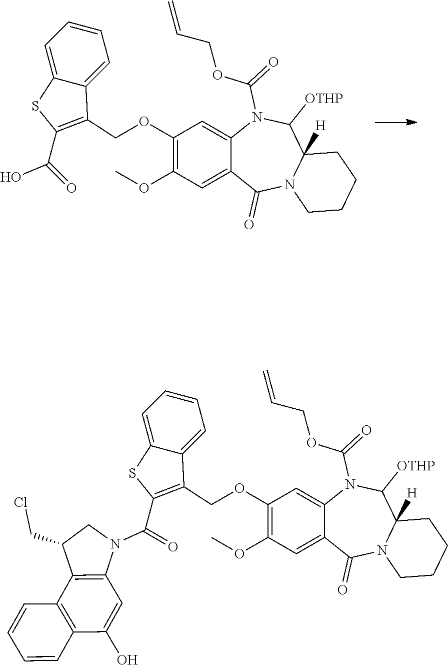

General Procedure C was followed, using 3-((((6aS)-5-((Allyloxy)carbonyl)-2-methoxy-12-oxo-6-((tetrahydro-2H-pyran-2-yl)oxy)-5,6,6a,7,8,9,10,12-octahydrobenzo[e]-pyrido[1,2-a][1,4]diazepin-3-yl)oxy)methyl)benzo[b]thiophene-2-carboxylic acid (89) (200 mg, 0.314 mmol), (S)-1-(chloromethyl)-2,3-dihydro-1H-benzo[e]indol-5-ol hydrochloride (11) (96 mg, 0.354 mmol), N-(3-dimethylaminopropyl)-N′-ethylcarbodiimide hydrochloride (203 mg, 1.06 mmol) and N,N-dimethylaectamide (1 mL). Chromatography, eluting with ethyl acetate/petroleum spirit, 40-60° C. (58%) gave the title compound (150 mg, 56%) as a green solid.

¹H NMR (400 MHz, CDCl₃) δ 9.63 (br, 1H), 8.17 (t, J=7.4 Hz, 1H), 8.00 (dd, J=11.7, 5.5 Hz, 1H), 7.83 (dd, J=12.6, 5.7 Hz, 1H), 7.58 (d, J=8.3 Hz, 1H), 7.44 (dt, J=70.4, 5.1 Hz, 3H), 7.33-7.25 (m, 1H), 7.22-6.90 (m, 3H), 6.12 (d, J=9.3 Hz, 1H), 5.94 (d, J=10.0 Hz, 1H), 5.59 (ddd, J=15.8, 10.6, 5.2 Hz, 1H), 5.46 (q, J=12.2 Hz, 2H), 5.01 (d, J=14.9 Hz, 1H), 4.92 (d, J=11.1 Hz, 1H), 4.51-4.16 (m, 4H), 3.91-3.67 (m, 3H), 3.58-3.51 (m, 3H), 3.49-3.31 (m, 3H), 3.12-2.93 (m, 1H), 1.93-1.14 (m, 12H); ¹³C NMR (100 MHz, CDCl₃) δ 169.3, 163.2, 155.8, 155.1, 149.8, 149.6, 149.5, 149.4, 14.6, 139.4, 137.9, 134.4, 132.1, 131.9, 129.9, 127.6, 125.2, 124.0, 123.7, 123.5, 123.2, 122.8, 122.1, 117.0, 116.2, 115.6, 110.8, 110.5, 100.0, 94.9, 88.1, 66.3, 64.4, 63.8, 62.9, 55.8, 55.5, 46.2, 42.1, 38.8, 30.9, 25.2, 25.0, 23.2, 22.9, 19.1; MS (ES+): m/z=852 (M+H)⁺; LCMS (Method A): t_{R}=8.93 min.

### Example 93: (S)-3-((2-((S)-1-(Chloromethyl)-5-hydroxy-2,3-dihydro-1H-benzo[e]indole-3-carbonyl)benzo[b]thiophen-3-yl)methoxy)-2-methoxy-7,8,9,10-tetrahydrobenzo[e]pyrido[1,2-a][1,4]diazepin-12(6aH)-one (91

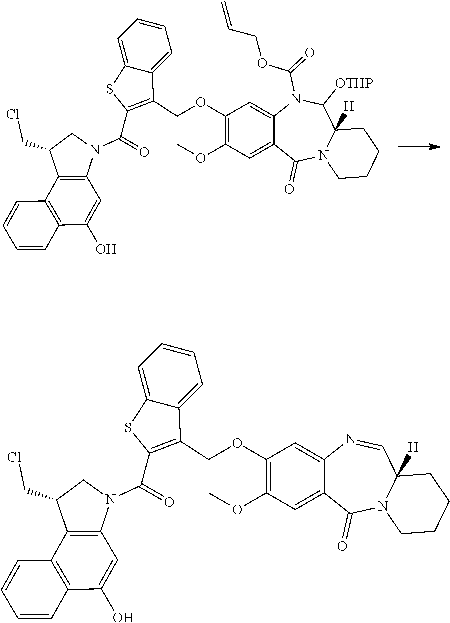

General Procedure D was followed, using allyl (6aS)-3-((2-((S)-1-(chloromethyl)-5-hydroxy-2,3-dihydro-1H-benzo[e]indole-3-carbonyl)benzo[b]thiophen-3-yl)methoxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (90) (150 mg, 0.176 mmol), tetrakis(triphenylphosphine)palladium(0) (20 mg, 0.018 mmol), pyrrolidine (18 μL, 0.211 mmol) and dichloromethane (1 mL). Chromatography, eluting with ethyl acetate/petroleum spirit, 40-60° C. (89%), followed by trituration from dichloromethane/diethyl ether, gave the title compound (43 mg, 37%) as an off-white solid.

¹H NMR (400 MHz, acetone-d₆) δ 9.37 (br, 1H), 8.28-8.16 (m, 2H), 8.09-8.04 (m, 1H), 7.85 (d, J=8.3 Hz, 2H), 7.60-7.49 (m, 4H), 7.43-7.36 (m, 1H), 7.25 (s, 1H), 6.97 (s, 1H), 5.58 (s, 2H), 4.45-4.27 (m, 2H), 4.17-4.06 (m, 2H), 4.00 (d, J=11.2 Hz, 1H), 3.84-3.73 (m, 1H), 3.63 (s, 3H), 3.51 (s, 1H), 3.10 (t, J=12.8 Hz, 1H), 1.85-1.55 (m, 6H); MS (ES+): m/z=666 (M+H)⁺; LCMS (Method A): t_{R}=7.45 min;

### Example 94: Allyl (6aS)-2-methoxy-3-((6-(2-methoxy-2-oxoethyl)pyridin-2-Y1)methoxy)-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (92)

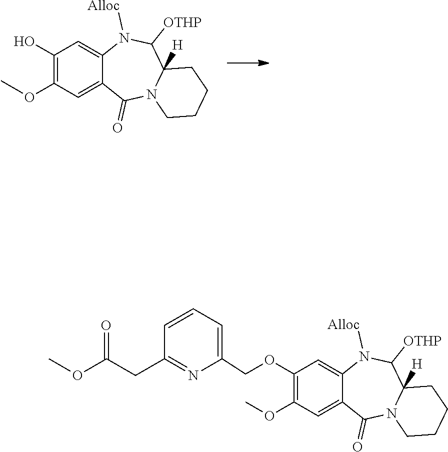

General Procedure A was followed, using allyl (6aS)-3-hydroxy-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (73) (100 mg, 0.224 mmol), methyl 2-[6-(chloromethyl)-2-pyridyl]acetate hydrochloride (46 mg, 0.195 mmol), potassium carbonate (61 mg, 0.44 mmol) and N,N-dimethylformamide (2 mL). Chromatography, eluting with ethyl acetate/petroleum spirit, 40-60° C. (84%) gave the title compound (100 mg, impure) as a white solid.

MS (ES+): m/z=610 (M+H)⁺; LCMS (Method C): t_{R}=3.93 min

### Example 95: 2-(6-((((6aS)-5-((Allyloxy)carbonyl)-2-methoxy-12-oxo-6-((tetrahydro-2H-pyran-2-yl)oxy)-5,6,6a,7,8,9,10,12-octahydrobenzo[e]-pyrido[1,2-a][1,4]diazepin-3-yl)oxy)methyl)pyridin-2-yl)acetic acid (93)

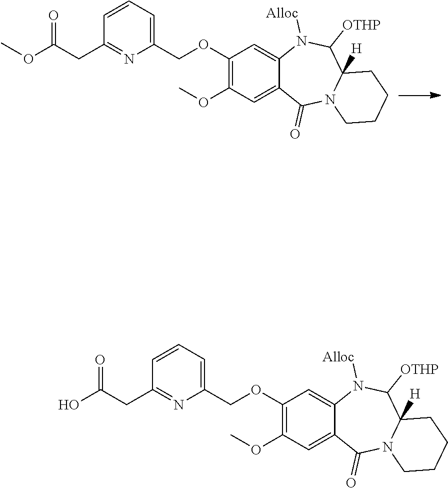

General Procedure B was followed, using allyl (6aS)-2-methoxy-3-((6-(2-methoxy-2-oxoethyl)pyridin-2-yl)methoxy)-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (92) (100 mg, 0.16 mmol), sodium hydroxide (0.5 M, aq., 0.65 mL, 0.32 mmol) and tetrahydrofuran (3 mL). The title compound (95 mg, impure) was isolated as a white solid, which was used in the next step without further purification.

MS (ES+): m/z=596 (M+H)⁺; LCMS (Method C): t_{R}=3.55 min.

### Example 96: Allyl (6aS)-3-((6-(2-((S)-1-(chloromethyl)-5-hydroxy-1,2-dihydro-3H-benzo[e]indol-3-yl)-2-oxoethyl)pyridin-2-yl)methoxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (94)

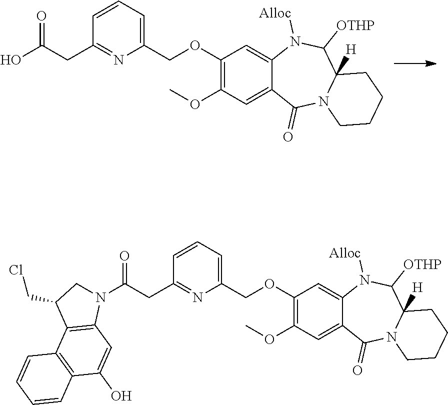

General Procedure C was followed, using 2-(6-((((6aS)-5-((allyloxy)carbonyl)-2-methoxy-12-oxo-6-((tetrahydro-2H-pyran-2-yl)oxy)-5,6,6a,7,8,9,10,12-octahydrobenzo[e]pyrido[1,2-a][1,4]diazepin-3-yl)oxy)methyl)pyridin-2-yl)acetic acid (93) (95 mg, 0.16 mmol), (S)-1-(Chloromethyl)-2,3-dihydro-1H-benzo[e]indol-5-ol hydrochloride (11) (43 mg, 0.16 mmol), N-(3-dimethylaminopropyl)-N′-ethylcarbodiimide hydrochloride (123 mg, 0.64 mmol) and N,N-dimethylaectamide (2 mL). Chromatography, eluting with ethyl acetate/petroleum spirit, 40-60° C. (87%) gave the title compound (43 mg, 33%) as a green solid.

¹H NMR (400 MHz, CDCl₃), mixture of diastereomers, δ8.26 (dd, J=14.3, 8.9 Hz, 1H), 8.00 (s, 1H), 7.68 (t, J=70.8 Hz, 1H), 7.59 (dd, J=14.1, 6.0 Hz, 1H), 7.48 (t, J=9.7 Hz, 1H), 7.42-7.31 (m, 2H), 7.20-7.14 (m, 1H), 7.04 (d, J=7.7 Hz, 1H), 6.85 (br, 1H), 6.17-6.10 (m, 1H), 6.01-5.90 (m, 1H), 5.68-5.52 (m, 1H), 5.27-5.16 (m, 2H), 5.07-4.89 (m, 2H), 4.76-4.69 (m, 1H), 4.48 (d, J=90.6 Hz, 1H), 4.36-4.24 (m, 1H), 4.22-4.02 (m, 2H), 3.96-3.84 (m, 4H), 3.85-3.67 (m, 2H), 3.58-3.33 (m, 2H), 3.28 (td, J=10.7, 4.7 Hz, 1H), 3.12-2.96 (m, 1H), 2.93 (s, 3H), 1.96-1.33 (m, 12H); ¹³C NMR (100 MHz, CDCl₃), mixture of diastereomers, δ169.3, 162.5, 156.8, 156.0, 155.7, 155.2, 149.2, 141.0, 137.9, 137.1, 132.1, 129.9, 127.5, 123.9, 123.4, 122.9, 122.4, 122.2, 122.0, 117.0, 114.9, 110.5, 100.7, 100.1, 88.0, 71.4, 66.5, 66.3, 63.0, 57.8, 56.1, 53.8, 52.2, 50.6, 46.0, 38.9, 36.5, 31.4, 30.9, 25.2, 23.2, 18.4; MS (ES+): m/z=811 (M+H)⁺; LCMS (Method C): t_{R}=4.38 min.

### Example 97: (S)-3-((6-(2-((S)-1-(Chloromethyl)-5-hydroxy-1,2-dihydro-3H-benzo[e]indol-3-yl)-2-oxoethyl)pyridin-2-yl)methoxy)-2-methoxy-7,8,9,10-tetrahydrobenzo[e]pyrido[1,2-a][1,4]diazepin-12(6aH)-one (95)

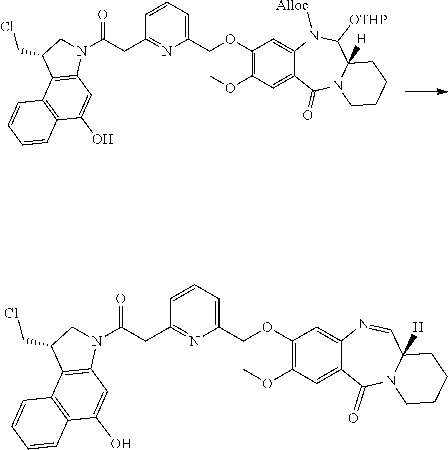

General Procedure D was followed, using allyl (6aS)-3-((6-(2-((S)-1-(chloromethyl)-5-hydroxy-1,2-dihydro-3H-benzo[e]indol-3-yl)-2-oxoethyl)pyridin-2-yl)methoxy)-2-methoxy-12-oxo-6-((tetrahydro-2H-pyran-2-yl)oxy)-6,6a,7,8,9,10-hexahydrobenzo[e]-pyrido[1,2-a][1,4]diazepine-5(12H)-carboxylate (94) (43 mg, 0.050 mmol), tetrakis(triphenylphosphine)palladium(0) (6 mg, 0.005 mmol), pyrrolidine (5 μL, 0.211 mmol) and dichloromethane (1 mL). Chromatography, eluting with ethyl acetate/petroleum spirit, 40-60° C. (from 75% to 100%), followed by ethyl acetate/methanol (7%), gave the title compound (12 mg, 36%) as a white solid.

¹H NMR (400 MHz, acetone-d₆) δ 9.43 (br, 1H), 8.21 (d, J=8.4 Hz, 1H), 8.06 (s, 1H), 7.84 (d, J=3.0 Hz, 1H), 7.83 (s, 1H), 7.81 (br, 1H), 7.55-7.52 (m, 1H), 7.51 (d, J=8.2 Hz, 1H), 7.41 (d, J=70.8 Hz, 1H), 7.37-7.32 (m, 2H), 6.84 (s, 1H), 5.32-5.23 (m, 2H), 4.69 (dd, J=10.8, 1.8 Hz, 1H), 4.50-4.43 (m, 1H), 4.13 (ddd, J=15.7, 12.9, 6.3 Hz, 4H), 3.97 (dd, J=10.9, 3.1 Hz, 1H), 3.87 (s, 3H), 3.79-3.76 (m, 1H), 3.73-3.65 (m, 2H), 1.96-1.53 (m, 6H); MS (ES+): m/z=625 (M+H)⁺; LCMS (Method A): t_{R}=6.97 min.

### Example 98: Allyl (6aS)-3-((6-ethoxy-6-oxohexyl)oxy)-2-methoxy-12-oxo-6-((tetrahydro-2H-pyran-2-yl)oxy)-6,6a,7,8,9,10-hexahydrobenzo[e]-pyrido[1,2-a][1,4]diazepine-5(12H)-carboxylate (96)

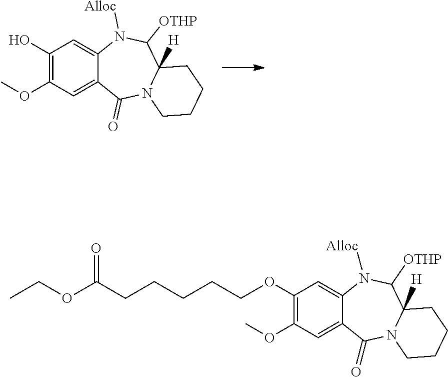

General Procedure A was followed, using allyl (6aS)-3-hydroxy-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (73) (670 mg, 1.50 mmol), ethyl 6-bromohexanoate (280 μL, 1.58 mmol), potassium carbonate (311 mg, 2.25 mmol) and N,N-dimethylformamide (2 mL). Chromatography, eluting with ethyl acetate/petroleum spirit, 40-60° C. (55%) gave the title compound (749 mg, 85%) as a yellow oil.

¹H NMR (400 MHz, CDCl₃), mixture of diastereomers, δ 7.16 (m, 1H), 6.50 (s, 1H), 6.10 (m, 1H), 5.81-5.76 (m, 1H), 5.14-5.03 (m, 2H), 4.69-4.57 (m, 2H), 4.47-4.37 (m, 1H), 4.34-4.26 (m, 1H), 4.12 (q, J=7.1 Hz, 2H), 4.01-3.94 (m, 3H), 3.90 (s, 3H), 3.68-3.62 (m, 1H), 3.68-3.46 (m, 2H), 3.12-3.03 (m, 1H), 2.33 (t, J=7.4 Hz, 2H), 1.89-1.66 (m, 11H), 1.57-1.47 (m, 6H), 1.25 (t, J=7.1 Hz, 3H); ¹³C NMR (100 MHz, CDCl₃), mixture of diastereomers, δ173.5, 173.4, 169.3, 169.1, 155.8, 150.0, 149.3, 149.0, 132.2, 132.0, 127.7, 126.2, 125.7, 116.9, 114.2, 113.7, 110.6, 110.2, 100.0, 98.9, 95.3, 88.0, 84.2, 68.8, 68.6, 66.2, 65.8, 63.9, 63.1, 60.1, 56.1, 56.0, 55.3, 55.2, 38.7, 34.2, 34.1, 31.0, 30.7, 28.6, 28.5, 25.5, 25.2, 25.0, 23.2, 23.1, 23.0, 22.9, 19.9, 19.6, 18.3, 18.1, 14.2; MS (ES+): m/z=589 (M+H)⁺; LCMS (Method B): t_{R}=4.32 min.

### Example 99: 6-(((6aS)-5-((Allyloxy)carbonyl)-2-methoxy-12-oxo-6-((tetrahydro-2H-pyran-2-yl)oxy)-5,6,6a,7,8,9,10,12-octahydrobenzo[e]-pyrido[1,2-a][1,4]diazepin-3-yl)oxy)hexanoic acid (97)

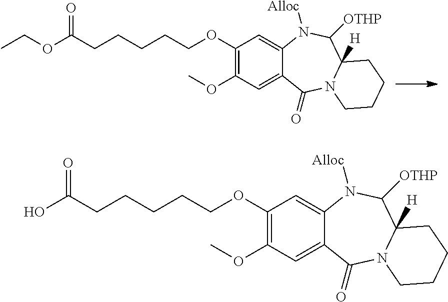

General Procedure B was followed, using allyl (6aS)-3-((6-ethoxy-6-oxohexyl)oxy)-2-methoxy-12-oxo-6-((tetrahydro-2H-pyran-2-yl)oxy)-6,6a,7,8,9,10-hexahydrobenzo[e]-pyrido[1,2-a][1,4]diazepine-5(12H)-carboxylate (96) (713 mg, 1.21 mmol), sodium hydroxide (0.5 M, aq., 6.06 mL, 3.03 mmol) and tetrahydrofuran (14 mL). The title compound (412 mg, 61%) was isolated as a white solid, which was used in the next step without further purification.

¹H NMR (400 MHz, CDCl₃) δ 7.18 (s, 1H), 6.19 (s, 1H), 6.18-5.99 (m, 1H), 5.81-5.71 (m, 1H), 5.12-5.02 (m, 2H), 4.67-4.51 (m, 1H), 4.48-4.36 (m, 1H), 4.31-4.23 (m, 1H), 4.00-3.88 (m, 7H), 3.66-3.46 (m, 2H), 3.12-3.02 (m, 1H), 2.36 (t, J=7.4 Hz, 2H), 1.81-1.79 (m, 2H), 1.75-1.65 (m, 10H), 1.55-1.49 (m, 7H); MS (ES+): m/z=561 (M+H)⁺; LCMS (Method B): t_{R}=3.78 min.

### Example 100: Allyl (6aS)-3-((6-((S)-1-(chloromethyl)-5-hydroxy-1,2-dihydro-3H-benzo[e]indol-3-yl)-6-oxohexyl)oxy)-2-methoxy-12-oxo-6-((tetrahydro-2H-pyran-2-yl)oxy)-6,6a,7,8,9,10-hexahydrobenzo[e]pyrido-[1,2-a][1,4]diazepine-5(12H)-carboxylate (98)

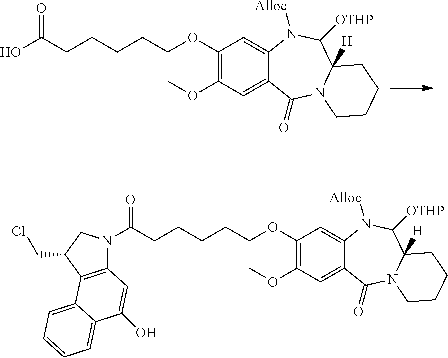

General Procedure C was followed, using 6-(((6aS)-5-((Allyloxy)carbonyl)-2-methoxy-12-oxo-6-((tetrahydro-2H-pyran-2-yl)oxy)-5,6,6a,7,8,9,10,12-octahydrobenzo[e]-pyrido[1,2-a][1,4]diazepin-3-yl)oxy)hexanoic acid (97) (400 mg, 0.713 mmol), (S)-1-(Chloromethyl)-2,3-dihydro-1H-benzo[e]indol-5-ol hydrochloride (11) (193 mg, 0.713 mmol), N-(3-dimethylaminopropyl)-N′-ethylcarbodiimide hydrochloride (537 mg, 2.80 mmol) and N,N-dimethylaectamide (3 mL). Chromatography, eluting with ethyl acetate/petroleum spirit, 40-60° C. (70%) gave the title compound (337 mg, 61%) as a grey solid.

¹H NMR (400 MHz, CDCl₃) δ 8.34 (d, J=70.2 Hz, 1H), 8.29 (d, J=8.2 Hz, 1H), 7.65 (d, J=8.4 Hz, 1H), 7.54-7.48 (m, 1H), 7.39-7.33 (m, 1H), 7.18 (s, 1H), 6.58 (s, 1H), 6.19 (d, J=10.0 Hz, 1H), 6.01 (d, J=10.0 Hz, 1H), 5.81-5.66 (m, 1H), 5.17-4.99 (m, 3H), 4.68-4.42 (m, 2H), 4.35-4.24 (m, 3H), 4.09-4.01 (m, 3H), 3.88 (s, 3H), 3.85-3.80 (m, 1H), 3.67-3.60 (m, 1H), 3.52-3.46 (m, 1H), 3.42 (t, J=11 Hz, 1H), 3.13-3.02 (m, 1H), 2.74-2.55 (m, 2H), 2.01-1.88 (m, 6H), 1.82-1.61 (m, 12H); ¹³C NMR (100 MHz, CDCl₃) δ 171.2, 163.7, 156.3, 155.1, 149.3, 146.6, 141.2, 131.6, 130.0, 127.6, 125.4, 123.9, 123.5, 122.7, 122.0, 117.9, 116.0, 114.6, 110.5, 108.0, 106.4, 100.4, 94.7, 69.1, 66.8, 63.0, 60.4, 56.1, 55.9, 52.3, 46.9, 46.3, 42.3, 35.7, 31.9, 29.7, 29.4, 25.5, 25.4, 25.2, 23.1, 22.7; MS (ES−): m/z=774 (M−1)⁻, MS (ES+): m/z=798 (M+Na)⁺; LCMS (Method C): t_{R}=3.93 min.

### Example 101: (S)-3-((6-((S)-1-(Chloromethyl)-5-hydroxy-1,2-dihydro-3H-benzo[e]indol-3-Y1)-6-oxohexyl)oxyl)-2-methoxy-7,8,9,10-tetrahydrobenzo-[e]pyrido[1,2-a][1,4]diazepin-12(6aH)-one (99)

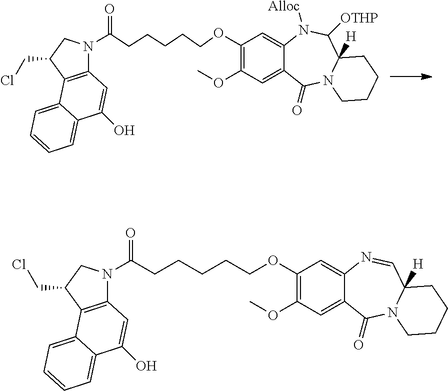

General Procedure D was followed, using allyl (6aS)-3-((6-((S)-1-(chloromethyl)-5-hydroxy-1,2-dihydro-3H-benzo [e]indol-3-yl)-6-oxohexyl)oxy)-2-methoxy-12-oxo-6-((tetrahydro-2H-pyran-2-yl)oxy)-6,6a,7,8,9,10-hexahydrobenzo[e]pyrido[1,2-a][1,4]-diazepine-5(12H)-carboxylate (98) (337 mg, 0.434 mmol), tetrakis(triphenyl-phosphine)palladium(0) (50 mg, 0.043 mmol), pyrrolidine (43 μL, 0.521 mmol) and dichloromethane (1 mL). Chromatography, eluting with ethyl acetate/petroleum spirit, 40-60° C. (from 30% to 100%), gave the title compound (161 mg, 63%) as a white solid.

¹H NMR (400 MHz, acetone-d₆) δ 9.31 (br s, 1H), 8.21 (d, J=8.2 Hz, 1H), 8.13 (s, 1H), 7.97 (d, J=5-5 Hz, 1H), 7.80 (d, J=8.6 Hz, 1H), 7.74-7.67 (m, 1H), 7.63-7.49 (m, 1H), 7.33 (s, 1H), 6.78 (s, 1H), 4.39-4.30 (m, 2H), 4.19-4.11 (m, 3H), 4.10-4.05 (m, 1H), 4.01 (dd, J=11.0, 3.5 Hz, 1H), 3.85 (s, 3H), 3.79-3.68 (m, 2H), 3.16 (td, J=11.3, 3.1 Hz, 1H), 2.70-2.56 (m, 2H), 2.18-2.10 (m, 1H), 2.02-1.95 (m, 1H), 1.94-1.76 (m, 6H), 1.70-1.60 (m, 4H); ¹³C NMR (100 MHz, acetone-d₆) δ 166.8, 163.9, 154.4, 151.0, 147.9, 140.3, 130.3, 127.2, 123.3, 122.7, 122.3, 121.1, 114.4, 111.7, 109.8, 100.4, 68.5, 55.4, 53.0, 49.6, 46.9, 41.7, 39.1, 35.3, 25.5, 24.1, 24.0, 22.9, 18.1; MS (ES+): m/z=590 (M+H)⁺; LCMS (Method A): t_{R}=7.20 min.

### Example 102: Allyl (6aS)-2-methoxy-3-((8-methoxy-8-oxooctyl)oxy)-12-oxo-6-((tetrahydro-2H-pyran-2-yl)oxy)-6,6a,7,8,9,10-hexahydrobenzo[e]-pyrido[1,2-a][1,4]diazepine-5(12H)-carboxylate (100)

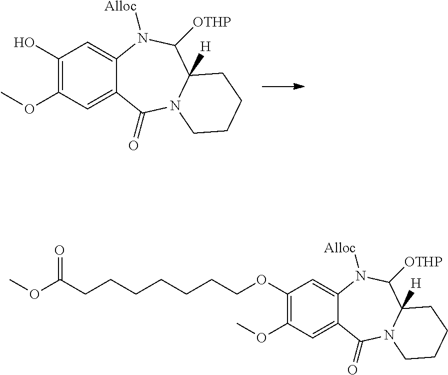

General Procedure A was followed, using allyl (6aS)-3-hydroxy-2-methoxy-12-oxo-6-((tetrahydro-2H-pyran-2-yl)oxy)-6,6a,7,8,9,10-hexahydrobenzo[e]pyrido[1,2-a][1,4]-diazepine-5(12H)-carboxylate (73) (150 mg, 0.34 mmol), methyl 8-bromooctanoate (70 μL, 0.36 mmol), potassium carbonate (70 mg, 0.51 mmol) and N,N-dimethylform-amide (2 mL). Chromatography, eluting with ethyl acetate/petroleum spirit, 40-60° C. (from 10% to 60%) gave the title compound (182 mg, 91%) as a yellow oil.

¹H NMR (400 MHz, CDCl₃), mixture of diastereomers, δ 7.14 (s, 1H), 7.11 (s, 1H), 6.80 (br, 1H), 6.16 (d, J=10.0 Hz, 1H), 5.98 (d, J=10.0 Hz, 1H), 5.74 (ddd, J=16.1, 10.4, 5.0 Hz, 1H), 5.06 (dd, J=23.1, 12.6 Hz, 3H), 4.68-4.52 (m, 3H), 4.48-4.34 (m, 2H), 4.30-4.20 (m, 1H), 3.99-3.92 (m, 4H), 3.49-3.42 (m, 1H), 3.08-2.97 (m, 2H), 2.27 (t, J=70.5 Hz, 2H), 1.86-1.31 (m, 22H); ¹³C NMR (100 MHz, CDCl₃), mixture of diastereomers, δ 174.1, 169.4, 169.2, 155.8, 150.1, 149.3, 149.0, 132.2, 132.0, 127.8, 126.1, 116.9, 114.2, 113.7, 110.6, 110.2, 100.1, 88.1, 84.2, 69.1, 68.9, 66.4, 65.8, 63.9, 63.2, 56.1, 56.0, 55.3, 51.4, 38.8, 38.7, 34.0, 33.9, 31.0, 29.0, 28.9, 28.8, 28.7, 25.8, 25.7, 25.2, 24.8, 24.7, 23.2, 23.0, 22.9, 19.9, 18.2; MS (ES+): m/z=603 (M+H)⁺; LCMS (Method C): t_{R}=4.55 min.

### Example 103: 8-(((6aS)-5-((Allyloxy)carbonyl)-2-methoxy-12-oxo-6-((tetrahydro-2H-pyran-2-yl)oxy)-5,6,6a,7,8,9,10,12-octahydrobenzo[e]pyrido[1,2-a][1,4]diazepin-3-yl)oxy)octanoic acid (101)

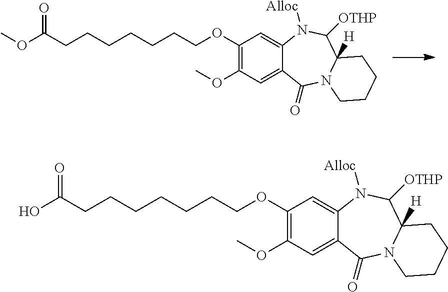

General Procedure B was followed, using allyl (6aS)-2-methoxy-3-((8-methoxy-8-oxooctyl)oxy)-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (too) (182 mg, 0.302 mmol), sodium hydroxide (0.5 M, aq., 12 mL, 0.604 mmol) and tetrahydrofuran (3 mL). The title compound (177 mg, quant.) was isolated as a white solid, which was used in the next step without further purification.

MS (ES+): m/z=589 (M+H)⁺; LCMS (Method C): t_{R}=4.03 min.

### Example 104: Allyl (6aS)-3-((8-((S)-1-(chloromethyl)-5-hydroxy-1,2-dihydro-3H-benzo[e]indol-3-yl)-8-oxooctyl)oxy)-2-methoxy-12-oxo-6-((tetrahydro-2H-pyran-2-yl)oxy)-6,6a,7,8,9,10-hexahydrobenzo[e]-pyrido[1,2-a][1,4]diazepine-5(12H)-carboxylate (102)

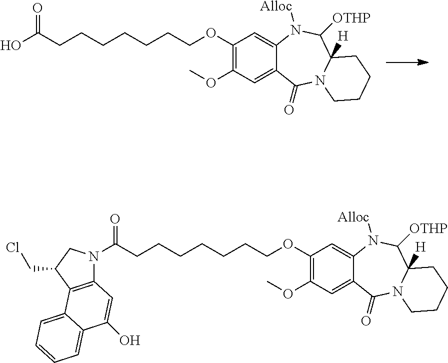

General Procedure C was followed, using 8-(((6aS)-5-((allyloxy)carbonyl)-2-methoxy-12-oxo-6-((tetrahydro-2H-pyran-2-yl)oxy)-5,6,6a,7,8,9,10,12-octahydrobenzo[e]pyrido[1,2-a][1,4]diazepin-3-yl)oxy)octanoic acid (45) (177 mg, 0.301 mmol), (S)-1-(Chloromethyl)-2,3-dihydro-1H-benzo[e]indol-5-ol hydrochloride (11) (81 mg, 0.301 mmol), N-(3-dimethylaminopropyl)-N′-ethylcarbodiimide hydrochloride (231 mg, 1.20 mmol) and N,N-dimethylaectamide (2 mL).

Chromatography, eluting with ethyl acetate/petroleum spirit, 40-60° C. (from 0% to 100%) gave the title compound (64 mg, 26%) as a grey solid.

¹H NMR (400 MHz, CDCl₃), mixture of diastereomers, δ10.16 (s, 1H), 8.39 (s, 1H), 8.29 (d, J=8.3 Hz, 1H), 7.62 (d, J=8.3 Hz, 1H), 7.55-7.45 (m, 1H), 7.43-7.31 (m, 1H), 7.17-7.12 (m, 1H), 6.84 (s, 1H), 6.50 (s, 1H), 6.18 (d, J=9.7 Hz, 1H), 6.00 (d, J=10.1 Hz, 1H), 5.80-5.68 (m, 1H), 5.17-4.98 (m, 2H), 4.70-4.51 (m, 1H), 4.50-4.13 (m, 4H), 3.95-3.88 (m, 7H), 3.66-3.43 (m, 2H), 3.39 (t, J=10.7 Hz, 1H), 3.11-3.01 (m, 1H), 2.66-2.45 (m, 2H), 1.97-1.39 (m, 22H); ¹³C NMR (100 MHz, CDCl₃), mixture of diastereomers, δ 173.1, 169.5, 169.3, 155.4, 149.4, 149.1, 141.2, 129.9, 128.9, 127.8, 127.5, 123.9, 123.3, 122.9, 121.9, 116.9, 114.5, 114.3, 100.6, 100.1, 88.1, 84.2, 69.2, 68.9, 66.5, 66.2, 63.9, 63.2, 62.6, 56.1, 53.5, 46.3, 42.2, 38.9, 36.4, 31.0, 29.7, 29.3, 29.2, 29.1, 28.9, 25.8, 25.7, 25.2, 24.9, 23.2, 23.0, 22.9, 19.9, 19.6; MS (ES+): m/z=804 (M+H)⁺; LCMS (Method C): t_{R}=4.75 min.

### Example 105: (S)-3-((8-((S)-1-(Chloromethyl)-5-hydroxy-1,2-dihydro-3H-benzo[e]indol-3-yl)-8-oxooctyl)oxy)-2-methoxy-7,8,9,10-tetrahydrobenzo[e]pyrido[1,2-a][1,4]diazepin-12(6aH)-one (103)

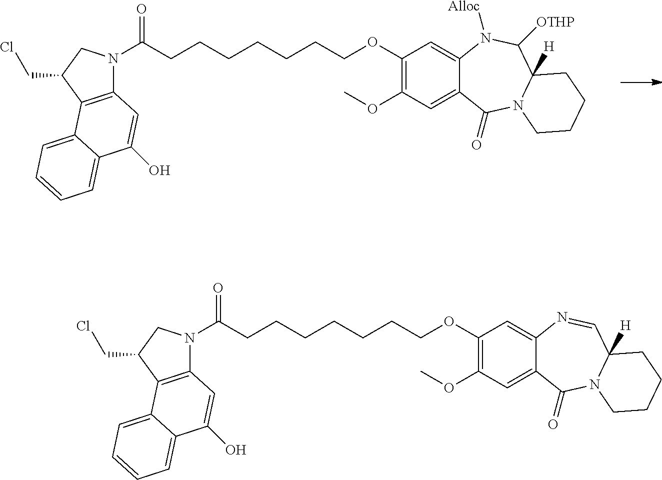

General Procedure D was followed, using allyl (6aS)-3-((8-((S)-1-(chloromethyl)-5-hydroxy-1,2-dihydro-3H-benzo [e]indol-3-yl)-8-oxooctyl)oxy)-2-methoxy-12-oxo-6-((tetrahydro-2H-pyran-2-yl)oxy)-6,6a,7,8,9,10-hexahydrobenzo[e]pyrido[1,2-a][1,4]-diazepine-5(12H)-carboxylate (102) (64 mg, 0.080 mmol), tetrakis(triphenyl-phosphine)palladium(0) (9 mg, 0.008 mmol), pyrrolidine (8 μL, 0.095 mmol) and dichloromethane (1 mL). Chromatography, eluting with ethyl acetate/petroleum spirit, 40-60° C. (97%), gave the title compound (8 mg, 16%) as a white solid.

¹H NMR (400 MHz, acetone-d₆) δ 9.36 (s, 1H), 8.20 (d, J=8.5 Hz, 1H), 8.10 (s, 1H), 7.96 (d, J=5.7 Hz, 1H), 7.78 (d, J=8.3 Hz, 1H), 7.49 (t, J=70.6 Hz, 1H), 7.32 (s, 1H), 7.31-7.27 (m, 2H), 6.75 (s, 1H), 4.34 (d, J=9.7 Hz, 2H), 4.16-4.08 (m, 2H), 4.05-3.95 (m, 2H), 3.86 (s, 3H), 3.79-3.75 (m, 1H), 3.73-3.66 (m, 1H), 3.19-3.11 (m, 1H), 2.66-2.48 (m, 2H), 1.88-1.70 (m, 8H), 1.58-1.42 (m, 8H); MS (ES+): m/z=618 (M+H)⁺; LCMS (Method A): t_{R}=7.70 min.

General Procedure E: Synthesis of Protected N-Methyl Piperazine Carbamates

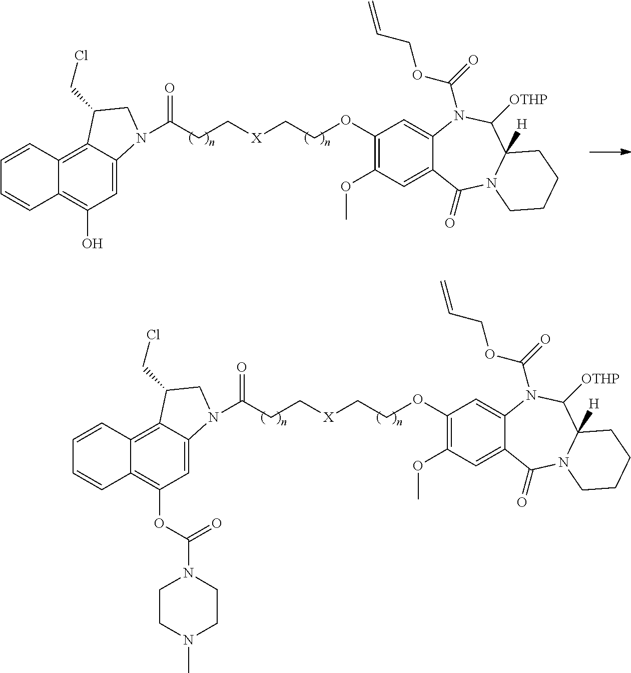

A solution of (S)-seco-CBI phenol (1 equiv.) in dichloromethane (0.1 M) was charged with 4-methyl-1-piperazinecarbonyl chloride hydrochloride (1 equiv.), followed by 4-(dimethylamino)pyridine (1.1 equiv.) and triethylamine (3.5 equiv.) and the resulting mixture stirred at room temperature. After the reaction was judged complete by TLC and LCMS (approx. 10 min), the mixture was concentrated in vacuo, then charged with diethyl ether and concentrated once again. The residue was then loaded directly onto silica and purified by flash column chromatography (eluent basified with 5% triethylamine).

General Procedure F: Deprotection of N-Alloc, O-THP pyridinobenzodiazepine N-methyl piperazine carbamates

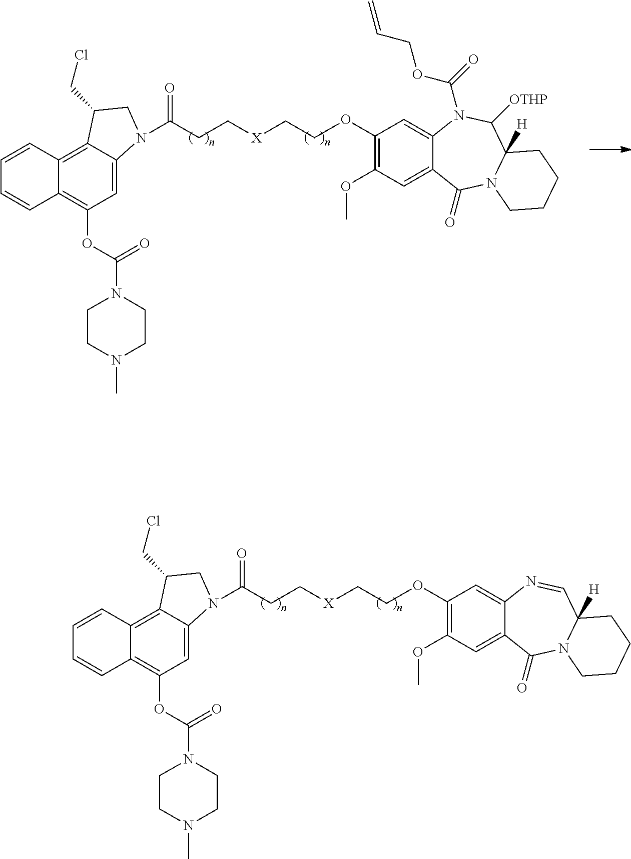

A solution of protected N-Alloc, O-THP pyridinobenzodiazepine N-methyl piperazine carbamate (1 equiv.) in dichloromethane (0.1 M) was charged with pyrrolidine (1.2 equiv.), and tetrakis(triphenylphosphine)palladium(0) (0.1 equiv.) and stirred at room temperature whilst monitoring by TLC and LCMS. After the reaction was judged to be complete (approx. 10 min), the mixture was diluted in dichloromethane and filtered through a pad of celite. The filtrate was concentrated in vacuo, then charged with diethyl ether and concentrated again. Diethyl ether was charged once more, and the residue concentrated in vacuo for a third time. The residue was then loaded directly onto silica and purified by flash column chromatography (eluent basified with approx. 5% triethylamine).

### Example 106: Allyl (6aS)-3-hydroxy-2,6-dimethoxy-14-oxo-6,6a,7,12-tetrahydrobenzo[5,6][1,4]diazepino[1,2-b]isoquinoline-5(14H)-carboxylate (104)

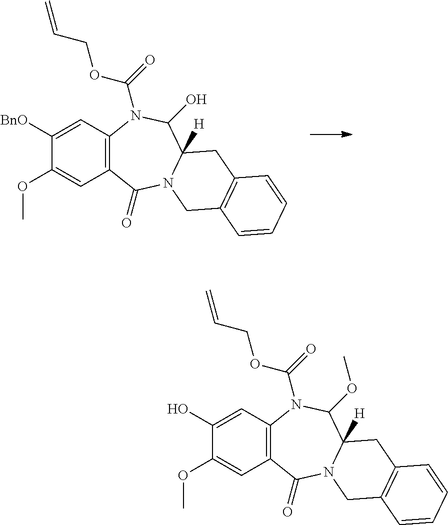 [SMILES: C=CCOC(=O)N1c2cc(OCc3ccccc3)c(OC)cc2C(=O)N2Cc3ccccc3C[C@H]2C1O ; C=CCOC(=O)N1c2cc(O)c(OC)cc2C(=O)N2Cc3ccccc3C[C@H]2C1OC]

A solution of allyl (6aS)-3-(benzyloxy)-6-hydroxy-2-methoxy-14-oxo-6,6a,7,12-tetrahydrobenzo[5,6][1,4]diazepino[1,2-b]isoquinoline-5(14H)-carboxylate (47) (100 mg, 0.199 mmol) in dichloromethane (1 mL) was charged with boron trichloride (1 M solution in dichloromethane, 600 μL, 0.600 mmol) and the resulting suspension was stirred at room temperature for 10 min, then methanol (2 mL) was added to the reaction mixture which was irradiated with microwaves 60 min at 55° C. The reaction mixture was subsequently filtered through a cotton pad that was washed with dichloromethane and concentrated in vacuo. Purification by flash column chromatography (silica), eluting with petroleum spirit 40-60° C./ethyl acetate (1:0 to 0:1) gave the title compound (40 mg, 48%) as a cream powder.

MS (ES+): m/z=424 (M+H)⁺; LCMS (Method B): t_{R}=3.53 min.

### Example 107: Allyl (6aS)-6-hydroxy-2-methoxy-3-((3-(2-methoxy-2-oxoethyl)benzyl)oxy)-14-oxo-6,6a,7,12-tetrahydrobenzo[5,6][1,4]-diazepino[1,2-b]isoquinoline-5(14H)-carboxylate (105)

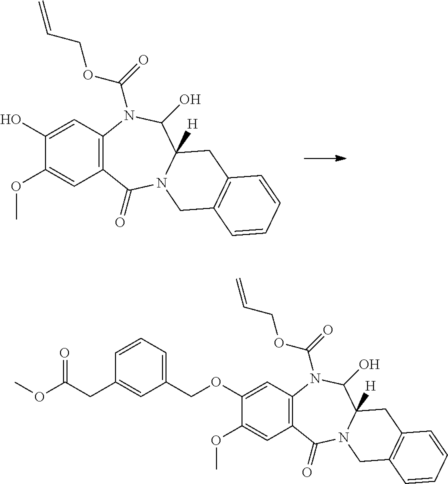 [SMILES: C=CCOC(=O)N1c2cc(O)c(OC)cc2C(=O)N2Cc3ccccc3C[C@H]2C1O ; C=CCOC(=O)N1c2cc(OCc3cccc(CC(=O)OC)c3)c(OC)cc2C(=O)N2Cc3ccccc3C[C@H]2C1O]

A solution of allyl (6aS)-3,6-dihydroxy-2-methoxy-14-oxo-6,6a,7,12-tetrahydrobenzo[5,6][1,4]diazepino[1,2-b]isoquinoline-5(14H)-carboxylate (104) (385 mg, 0.94 mmol) in N,N-dimethylformamide (1 mL) was charged with potassium carbonate (195 mg, 1.41 mmol) and 3-(bromomethyl)-benzeneacetic acid methyl ester (241 mg, 0.99 mmol) and the resulting mixture stirred for 16 h. The mixture was then diluted in ethyl acetate (50 mL) and washed with cold brine (2×20 mL), dried over magnesium sulfate, filtered, and concentrated in vacuo. Purification by flash column chromatography, eluting with ethyl acetate/petroleum spirit, 40-60° C. (70%) gave the title compound (202 mg, 32%) as a yellow solid.

¹H NMR (400 MHz, CDCl₃) δ 7.36-7.28 (m, 3H), 7.27-7.19 (m, 6H), 6.70 (s, 1H), 5.67 (br, 1H), 5.29 (dd, J=8.7, 5.0 Hz, 1H), 5.12-5.04 (m, 4H), 4.79 (d, J=15.7 Hz, 1H), 4.56 (d, J=15.5 Hz, 1H), 4.50-4.30 (m, 2H), 3.90 (s, 3H), 3.70-3.65 (m, 4H), 3.61 (d, J=7.1 Hz, 2H), 3.08 (qd, J=15.3, 4.8 Hz, 2H); ¹³C NMR (100 MHz, CDCl₃) δ 172.0, 168.9, 155.7, 150.1, 148.9, 136.5, 131.9, 129.1, 128.9, 128.2, 127.8, 127.7, 127.2, 126.6, 118.0, 114.3, 111.2, 84.8, 71.0, 66.6, 60.4, 56.2, 55.7, 52.1, 44.2, 40.9, 30.2, 21.0; MS (ES+): m/z=573 (M+H)⁺; LCMS (Method C): t_{R}=3.78 min.

### Example 108: 2-(3-((((6aS)-5-((Allyloxy)carbonyl)-6-hydroxy-2-methoxy-14-oxo-5,6,6a,7,12,14-hexahydrobenzo[5,6][1,4]diazepino[1,2-b]-isoquinolin-3-yl)oxy)methyl)phenyl)acetic acid (106)

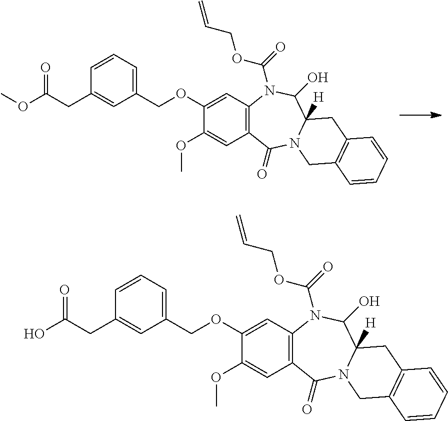 [SMILES: C=CCOC(=O)N1c2cc(OCc3cccc(CC(=O)OC)c3)c(OC)cc2C(=O)N2Cc3ccccc3C[C@H]2C1O ; C=CCOC(=O)N1c2cc(OCc3cccc(CC(=O)O)c3)c(OC)cc2C(=O)N2Cc3ccccc3C[C@H]2C1O]

A solution of allyl (6aS)-6-hydroxy-2-methoxy-3-((3-(2-methoxy-2-oxoethyl)benzyl)-oxy)-14-oxo-6,6a,7,12-tetrahydrobenzo[5,6][1,4]diazepino[1,2-b]isoquinoline-5(14H)-carboxylate (105) (200 mg, 0.35 mmol) in tetrahydrofuran (4.5 mL) was charged with an aqueous solution of sodium hydroxide (1 M, 900 μL, 0.90 mmol) and stirred for 2 h.

The mixture was then partially concentrated in vacuo, taken up into ethyl acetate (50 mL), and adjusted to pH=3-4 with an aqueous solution of citric acid (1 M). After separating the organic phase, the aqueous phase was extracted with ethyl acetate (50 mL) and the combined organic extracts dried over magnesium sulfate, filtered and concentrated in vacuo to give the title compound (175 mg, 90%) as a white solid, which was used in the subsequent step without further purification.

MS (ES+): m/z=559 (M+H)⁺; LCMS (Method C): t_{R}=3.52 min.

### Example 109: Allyl (6aS)-3-((3-(2-((S)-1-(chloromethyl)-5-hydroxy-1,2-dihydro-3H-benzo[e]indol-3-yl)-2-oxoethyl)benzyl)oxy)-6-hydroxy-2-methoxy-14-oxo-6,6a,7,12-tetrahydrobenzo [5,6][1,4]diazepino[1,2-b]-isoquinoline-5(14H)-carboxylate (107)

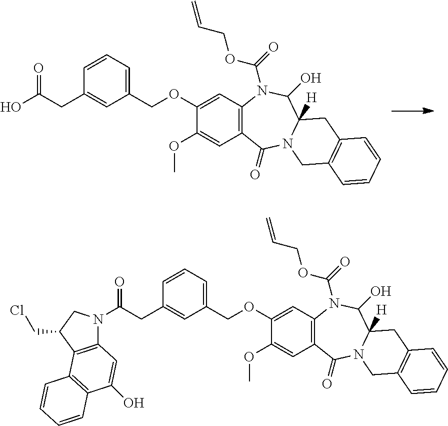 [SMILES: C=CCOC(=O)N1c2cc(OCc3cccc(CC(=O)O)c3)c(OC)cc2C(=O)N2Cc3ccccc3C[C@H]2C1O ; C=CCOC(=O)N1c2cc(OCc3cccc(CC(=O)N4C[C@@H](CCl)c5c4cc(O)c4ccccc54)c3)c(OC)cc2C(=O)N2Cc3ccccc3C[C@H]2C1O]

A solution of 2-(3-((((6aS)-5-((allyloxy)carbonyl)-6-hydroxy-2-methoxy-14-oxo-5,6,6a,7,12,14-hexahydrobenzo[5,6][1,4]diazepino[1,2-b]isoquinolin-3-yl)oxy)methyl)phenyl)acetic acid (106) (175 mg, 0.313 mmol) in N,N-dimethylacetamide (4 mL) was charged to (S)-1-(chloromethyl)-2,3-dihydro-1H-benzo[e]indol-5-ol hydrochloride (11) (95 mg, 0.352 mmol), followed by N-(3-dimethylaminopropyl)-N′-ethylcarbodiimide hydrochloride (268 mg, 1.40 mmol) and the resulting mixture stirred at room temperature for 16 h. The reaction mixture was subsequently diluted into ethyl acetate (50 mL) and washed with cold brine (2×20 mL), dried over magnesium sulfate, filtered and concentrated in vacuo. Flash column chromatography (silica), eluting with ethyl acetate/petroleum spirit, 40-60° C. (from 40% to 100%) afforded the title compound (114 mg, 42%) as an off-white solid.

¹H NMR (400 MHz, CDCl₃), mixture of diastereomers, δ9.07 (s, 1H), 8.90 (s, 1H), 8.25-8.19 (m, 2H), 8.14-8.07 (m, 1H), 7.66-7.58 (m, 1H), 7.56-7.46 (m, 1H), 7.45-7.30 (m, 4H), 7.27-7.18 (m, 4H), 7.12 (d, J=6.6 Hz, 1H), 6.75 (s, 1H), 5.63 (ddd, J=22.4, 10.4, 5.2 Hz, 1H), 5.47-5.30 (m, 1H), 5.28-5.20 (m, 1H), 5.08-4.98 (m, 3H), 4.71 (dd, J=15.6, 6.6 Hz, 1H), 4.58 (dd, J=15.3, 5.1 Hz, 1H), 4.53-4.43 (m, 1H), 4.42-4.29 (m, 1H), 4.42-4.29 (m, 2H), 4.26-4.16 (m, 1H), 3.97 (br, 1H), 3.96-3.82 (m, 5H), 3.48 (br, 1H), 3.37 (t, J=10.7 Hz, 1H), 2.96 (d, J=4.7 Hz, 2H); MS (ES+): m/z=774 (M+H)⁺; LCMS (Method C): t_{R}=4.18 min.

### Example 110: (S)-3-((3-(2-((S)-1-(Chloromethyl)-5-hydroxy-1,2-dihydro-3H-benzo[e]indol-3-yl)-2-oxoethyl)benzyl)oxy)-2-methoxy-7,12-dihydrobenzo[5,6][1,4]diazepino[1,2-b]isoquinolin-14(6aH)-one (108)

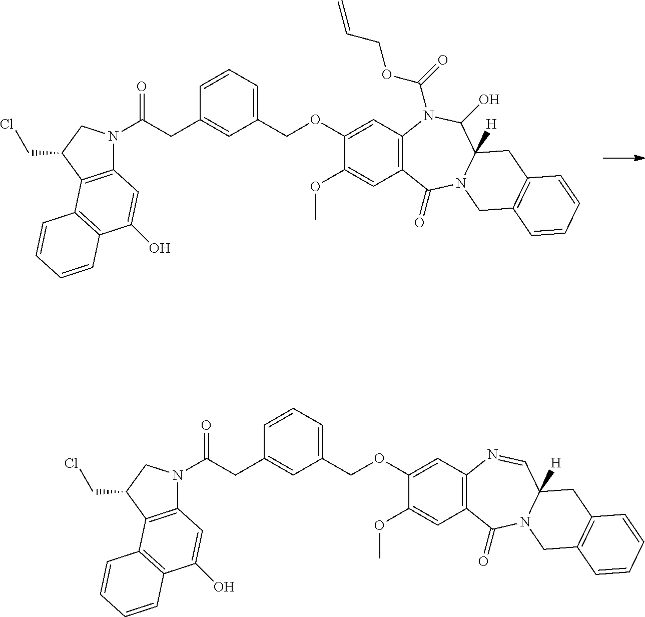 [SMILES: C=CCOC(=O)N1c2cc(OCc3cccc(CC(=O)N4C[C@@H](CCl)c5c4cc(O)c4ccccc54)c3)c(OC)cc2C(=O)N2Cc3ccccc3C[C@H]2C1O ; COc1cc2c(cc1OCc1cccc(CC(=O)N3C[C@@H](CCl)c4c3cc(O)c3ccccc43)c1)N=C[C@@H]1Cc3ccccc3CN1C2=O]

A solution of allyl (6aS)-3-((3-(2-((S)-1-(chloromethyl)-5-hydroxy-1,2-dihydro-3H-benzo[e]indol-3-yl)-2-oxoethyl)benzyl)oxy)-6-hydroxy-2-methoxy-14-oxo-6,6a,7,12-tetrahydrobenzo[5,6][1,4]diazepino[1,2-b]isoquinoline-5(14H)-carboxylate (106) (114 mg, 0.15 mmol) in dichloromethane (0.5 mL) was charged with tetrakis(triphenylphosphine)palladium(0) (17 mg, 0.015 mmol) and pyrrolidine (15 μL, 0.18 mmol) and stirred at room temperature. After the reaction was judged to be complete by TLC and LCMS, the mixture was concentrated in vacuo, charged with diethyl ether and concentrated in vacuo again. Purification by flash column chromatography (silica), eluting with ethyl acetate/petroleum spirit, 40-60° C. (from 50% to 100%) gave the title compound (39 mg, 39%) as a white solid.

¹H NMR (400 MHz, acetone-d₆) δ 9.76 (br, 1H), 8.20 (d, J=8.4 Hz, 1H), 8.06 (s, 1H), 7.80-7.74 (m, 1H), 7.52-7.49 (m, 1H), 7.47 (br, 1H), 7.47-7.45 (m, 2H), 7.44 (d, J=1.4 Hz, 1H), 7.42-7.40 (m, 2H), 7.38 (s, 1H), 7.37-7.29 (m, 3H), 7.28 (d, J=6.3 Hz, 1H), 6.87 (s, 1H), 5.21 (q, J=12.4 Hz, 2H), 4.88 (d, J=15.3 Hz, 1H), 4.59-4.49 (m, 1H), 4.41 (d, J=10.5 Hz, 1H), 4.36-4.28 (m, 1H), 4.10 (br, 1H), 4.00-3.95 (m, 1H), 3.93-3.91 (m, 1H), 3.88 (dd, J=11.3, 5.3 Hz, 1H), 3.83 (s, 3H), 3.73 (dd, J=5.3, 3.9 Hz, 1H), 3.66-3.60 (m, 1H), 3.29-3.25 (m, 2H); MS (ES+): m/z=672 (M+H)⁺; LCMS (Method A): t_{R}=7.80 min.

### Example 111: (S)-(2-(Hydroxymethyl)indolin-1-yl)(5-methoxy-2-nitro-4-((triisopropylsilyl)oxy)phenyl)methanone (109)

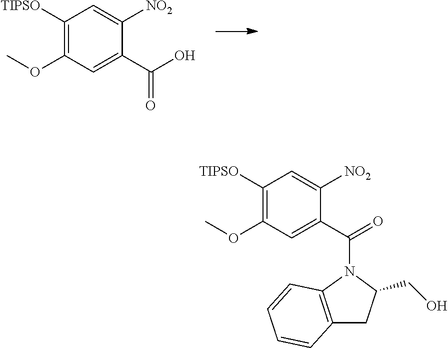

A solution of 5-methoxy-2-nitro-4-((triisopropylsilyl)oxy)benzoic acid (67) (1.00 g, 2.71 mmol) in dichloromethane (25 mL) was charged with (S)-(+)-2-indolinemethanol (404 mg, 2.71 mmol), HATU (0.54 g, 4.06 mmol) and N,N-diisopropylethylamine (875 mg, 6.77 mmol). The reaction mixture was stirred at room temperature for 3 h and then diluted with water (100 mL) and extracted with dichloromethane (100 mL×2). The combined organic extracts were then dried over anhydrous sodium sulfate, filtered and concentrated in vacuo. The residue was purified by flash column chromatography (silica), eluting with ethyl acetate/petroleum spirit, 40-60° C. (from 10% to 100%) to afford the title compound (800 mg, 58%) as a yellow oil.

¹H NMR (400 MHz, CDCl₃) δ 7.22-7.14 (m, 1H), 7.08-7.00 (m, 1H), 6.95-6.90 (m, 1H), 6.80-6.70 (m, 1H), 5.69-5.65 (m, 1H), 5.23-5.06 (m, 1H), 4.00-3.82 (m, 3H), 2.80 (s, 5H), 2.04 (s, 1H), 1.34-1.25 (m, 3H), 1.15-1.10 (m, 18H); MS (ES+): m/z=501 (M+H)⁺; LCMS (Method E): t_{R}=4.45 min.

### Example 112: (S)-(2-Amino-5-methoxy-4-((triisopropylsilyl)oxy)phenyl)(2-(hydroxymethyl)indolin-1-yl)methanone (110)

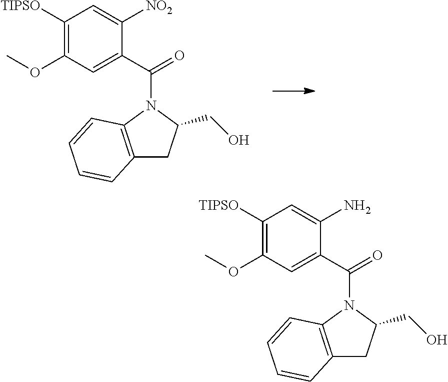

A solution of(S)-(2-(hydroxymethyl)indolin-1-yl)(5-methoxy-2-nitro-4-((triisopropylsilyl)oxy)phenyl)methanone (109) (800 mg, 1.60 mmol) in methanol (10 mL) was charged with palladium (10 wt. % loading on carbon, 80 mg). The mixture was stirred at room temperature under an atmosphere of hydrogen for 16 h then filtered through a pad of Celite. The resulting cake was then washed with ethyl acetate (50 mL) and concentrated under reduced pressure. The residue was then purified by flash column chromatography (silica), eluting with ethyl acetate/petroleum spirit, 40-60° C. (from 20% to 50%) to afford the title compound (500 mg, 66%) as a yellow oil.

¹H NMR (400 MHz, DMSO-d₆) δ 7.22 (d, J=6.8 Hz, 1H), 7.08 (s, 1H), 7.00-6.93 (m, 2H), 6.75 (s, 1H), 6.37 (d, J=2.8 Hz, 1H), 4.98-4.88 (m, 3H), 4.61-4.57 (m, 1H), 3.58 (s, 3H), 3.47-3.44 (m, 1H), 3.32-3.26 (m, 1H), 3.01-2.97 (m, 1H), 2.69 (s, 1H), 1.27-1.21 (m, 3H), 1.08 (d, J=70.2 Hz, 18H); MS (ES+): m/z=471 (M+H)⁺; LCMS (Method D): t_{R}=2.98 min.

### Example 113: Allyl (S)-(2-(2-(hydroxymethyl)indoline-1-carbonyl)-4-methoxy-5-((triisopropylsilyl)oxy)phenyl)carbamate (111)

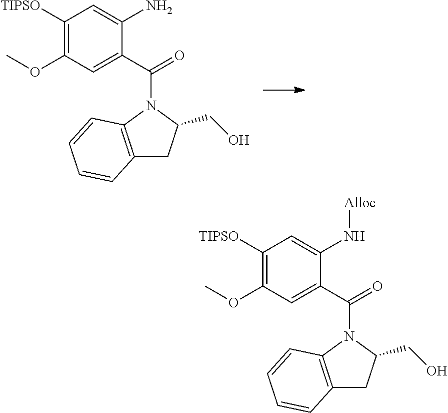

A solution of (S)-(2-amino-5-methoxy-4-((triisopropylsilyl)oxy)phenyl)(2-(hydroxymethyl)indolin-1-yl)methanone (110) (470 mg, 1.00 mmol) in dichloromethane (10 mL) at −10° C. was charged with anhydrous pyridine (158 mg, 2.00 mmol) and allyl chloroformate (127 mg, 1.05 mmol). After 30 min, the reaction was judged to be complete by TLC and was then diluted with dichloromethane (100 mL), washed with a saturated aqueous solution of copper sulfate (100 mL), water (100 mL) and a saturated aqueous solution of sodium hydrogen carbonate (10 mL). The organic layer was then dried over anhydrous sodium sulfate, filtered and concentrated in vacuo. The residue was purified by flash column chromatography (silica), eluting with ethyl acetate/petroleum spirit, 40-60° C. (5%) to afford the title compound (400 mg, 72%) as a yellow oil.

¹H NMR (400 MHz, CDCl₃) δ 8.36 (s, 1H), 7.74 (s, 1H), 7.19 (d, J=70.2 Hz, 1H), 6.97-6.87 (m, 2H), 6.72 (s, 1H), 6.40 (s, 1H), 5.98-5.87 (m, 1H), 5.31 (d, J=16.8 Hz, 1H), 5.22 (d, J=10.4 Hz, 1H), 4.94-4.91 (m, 1H), 4.60 (d, J=50.6 Hz, 2H), 3.76 (d, J=6.0 Hz, 2H), 3.54 (s, 3H), 3.45-3.38 (m, 1H), 2.81-2.76 (m, 1H), 1.35-1.28 (m, 3H), 1.12 (d, J=70.6 Hz, 18H); MS (ES+): m/z=555 (M+H)⁺; LCMS (Method D): t_{R}=2.78 min.

### Example 114: Allyl (12aS)-12-hydroxy-8-methoxy-6-oxo-A-((triisopropyl-silyl)oxy)-12a,13-dihydro-6H-benzo[5,6][1,4]diazepino[1,2-a]indole-11(12H)-carboxylate (112)

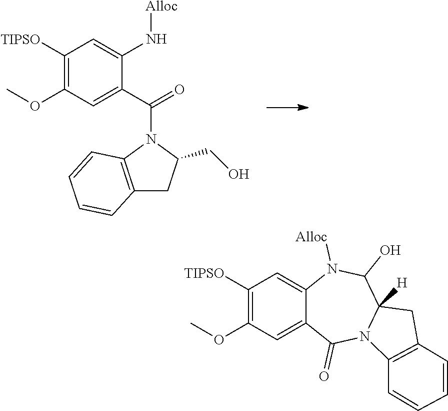

A solution of allyl (S)-(2-(2-(hydroxymethyl)indoline-1-carbonyl)-4-methoxy-5-((triisopropylsilyl)oxy)phenyl)carbamate (111) (391 mg, 0.71 mmol) in dichloromethane (13 mL) was charged with 2,2,6,6-tetramethylpiperidine 1-oxyl (11 mg, 0.07 mmol) and (diacetoxyiodo)benzene (274 mg, 0.85 mmol). The reaction mixture was stirred at room temperature for 18 h and then diluted in dichloromethane (40 mL), washed with a saturated aqueous solution of sodium metabisulfite (10 mL), then a saturated aqueous solution of sodium hydrogen carbonate (10 mL) and lastly, brine (10 mL). The organic layer was then dried over anhydrous sodium sulfate, filtered and concentrated in vacuo. The residue was purified by flash column chromatography (silica), eluting with ethyl acetate/petroleum spirit, 40-60° C. (10%) to afford the title compound (290 mg, 74%) as a yellow oil.

¹H NMR (500 MHz, CDCl₃) δ 8.19 (d, J=8.0 Hz, 1H), 7.21 (d, J=7.5 Hz, 1H), 7.08 (t, J=7.5 Hz, 1H), 6.71 (s, 1H), 5.78 (s, 1H), 5.73 (d, J=10.0 Hz, 1H), 5.20-5.14 (m, 2H), 4.62-4.58 (m, 1H), 4.46 (s, 1H), 4.15-4.06 (m, 2H), 3.86-3.84 (m, 3H), 3.49-3.43 (m, 1H), 3.21 (d, J=17.0 Hz, 1H), 2.04 (d, J=2.0 Hz, 1H), 1.28-1.21 (m, 3H), 1.09-1.08 (m, 18H); MS (ES+): m/z=553 (M+H)⁺; LCMS (Method D): t_{R}=2.68 min.

### Example 115: Allyl (12aS)-8-methoxy-6-oxo-12-((tetrahydro-2H-pyran-2-yl)oxy)-9-((triisopropylsilyl)oxy)-12a,13-dihydro-6H-benzo[5,6][1,4]-diazepino[1,2-a]indole-11(12H)-carboxylate (11)

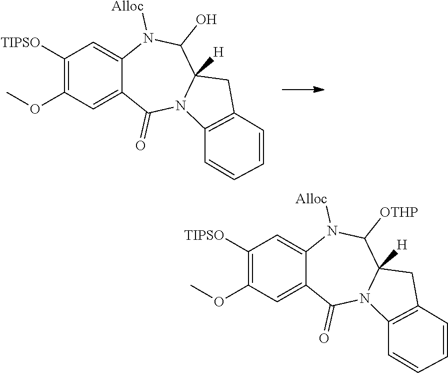

A solution of allyl (12aS)-12-hydroxy-8-methoxy-6-oxo-9-((triisopropylsilyl)oxy)-12a,13-dihydro-6H-benzo[5,6][1,4]diazepino[1,2-a]indole-11(12H)-carboxylate (112) (280 mg, 0.510 mmol) in tetrahydrofuran (5 mL) was charged with 3,4-dihydro-2H-pyran (429 mg, 5.10 mmol) and p-toluenesulfonic acid monohydrate (3 mg, 1% w/w). The reaction mixture was stirred at room temperature for 18 h and then diluted with ethyl acetate (30 mL), washed with a saturated aqueous solution of sodium hydrogen carbonate (10 mL) and brine (10 mL), then dried over anhydrous sodium sulfate, filtered and concentrated in vacuo. The residue was purified by flash column chromatography (silica), eluting with ethyl acetate/petroleum spirit, 40-60° C. (20%) to afford the title compound (300 mg, 92%) as a colourless oil.

¹H NMR (500 MHz, CDCl₃) δ 8.20-8.13 (m, 1H), 7.25-7.21 (m, 2H), 7.08-6.61 (m, 2H), 5.94 (m, 1H), 5.78-5.66 (m, 1H), 5.13-5.04 (m, 2H), 4.96-4.94 (m, 2H), 4.89 (d, J=6.0 Hz, 1H), 3.86 (d, J=2.0 Hz, 3H), 3.65-3.61 (m, 1H), 3.47-3.42 (m, 1H), 2.07-2.01 (m, 1H), 1.98-1.95 (m, 1H), 1.79-1.73 (m, 6H), 1.33-1.26 (m, 3H), 1.11-1.08 (m, 18H); MS (ES+): m/z=637 (M+H)⁺; LCMS (Method D): t_{R}=4.43 min.

### Example 116: Allyl (12aS)-Q-hydroxy-8-methoxy-6-oxo-12-((tetrahydro-2H-pyran-2-yl)oxy)-12a,13-dihydro-6H-benzo[5,6][1,4]diazepino[1,2-a]indole-11(12H)-carboxylate (114)

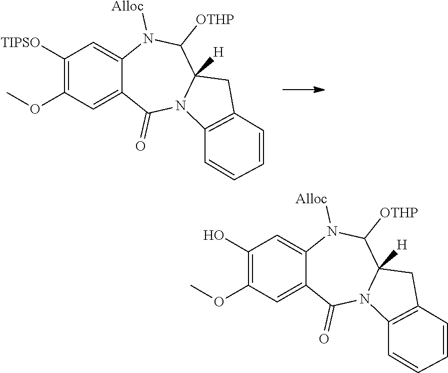

A solution of allyl (12aS)-8-methoxy-6-oxo-12-((tetrahydro-2H-pyran-2-yl)oxy)-9-((triisopropylsilyl)oxy)-12a,13-dihydro-6H-benzo[5,6][1,4]diazepino[1,2-a]indole-11(12H)-carboxylate (113) (290 mg, 0.46 mmol) in tetrahydrofuran (5 mL) under an inert atmosphere of nitrogen was charged with tetrabutylammonium fluoride (1 M in tetrahydrofuran, 0.65 mL, 0.65 mmol). The mixture was stirred at room temperature for 1 h and then charged with water (10 mL), extracted with ethyl acetate (30 mL×2) and the combined organic phases washed with brine (10 mL), dried over anhydrous sodium sulfate, filtered and concentrated in vacuo. The residue was purified by flash column chromatography (silica), eluting with ethyl acetate/petroleum spirit, 40-60° C. (20%) to afford the title compound (100 mg, 45%) as a white solid.

¹H NMR (500 MHz, DMSO-d₆) S8.16 (dd, J=21.0, 8.0 Hz, 1H), 7.28 (d, J=5.0 Hz, 1H), 7.25-7.20 (m, 1H), 7.10-7.05 (m, 1H), 6.77 (m, 1H), 6.02 (s, 1H), 5.81-5.72 (m, 1H), 5.20-5.14 (m, 1H), 5.13-4.84 (m, 1H), 4.66-4.48 (m, 2H), 4.15-4.07 (m, 1H), 3.95 (s, 3H), 3.60-3.42 (m, 2H), 3.30-3.18 (m, 1H), 1.88-1.54 (m, 7H), 1.29-1.24 (m, 1H); MS (ES+): m/z=481 (M+H)⁺; LCMS (Method E): t_{R}=2.78 min.

### Example 117: Allyl (12aS)-8-methoxy-9-((3-(2-methoxy-2-oxoethyl)benzyl)-oxy)-6-oxo-12-((tetrahydro-2H-pyran-2-yl)oxy)-12a,13-dihydro-6H-benzo-[5,6][1,4]diazepino[1,2-a]indole-11(12H)-carboxylate (15)

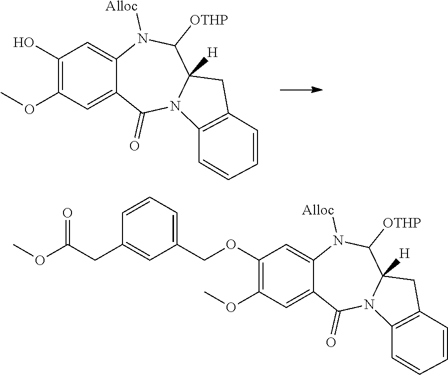

A solution of allyl (12aS)-9-hydroxy-8-methoxy-6-oxo-12-((tetrahydro-2H-pyran-2-yl)oxy)-12a,13-dihydro-6H-benzo[5,6][1,4]diazepino[1,2-a]indole-11(12H)-carboxylate (114) (250 mg, 0.52 mmol) in N,N-dimethylformamide (2 mL) was charged with potassium carbonate (108 mg, 0.78 mmol) and 3-(bromomethyl)-benzeneacetic acid methyl ester (134 mg, 0.55 mmol) and stirred at room temperature, whilst monitoring by TLC and LCMS. Once the reaction was judged to be complete, it was diluted into ethyl acetate and washed twice with cold brine. The organic phase was dried over magnesium sulfate, filtered and concentrated in vacuo, and then purified by flash column chromatography (silica), eluting with ethyl acetate/petroleum spirit, 40-60° C. (from 0% to 50%) to give the title compound (223 mg, 67%) as a white solid. ¹H NMR (400 MHz, CDCl₃), mixture of diastereomers, δ8.10 (dt, J=17.3, 8.5 Hz, 1H), 7.33-7.28 (m, 3H), 7.05-6.98 (m, 1H), 6.92 (s, 1H), 6.58 (br, 1H), 5.95 (d, J=90.2 Hz, 1H), 5.84 (d, J=90.6 Hz, 1H), 5.72-5.57 (m, 1H), 5.17-4.98 (m, 4H), 4.56-4.44 (m, 1H), 4.41 (br, 1H), 4.31 (d, J=90.6 Hz, 1H), 4.10-3.97 (m, 1H), 3.84-3.77 (m, 1H), 3.88 (s, 3H), 3.64 (s, 3H), 3.59 (s, 2H), 3.57-3.51 (m, 1H), 3.41 (dt, J=16.7, 10.9 Hz, 2H), 3.22 (d, J=17.3 Hz, 1H), 3.02 (d, J=16.2 Hz, 1H), 1.80-1.27 (m, 6H); ¹³C NMR (100 MHz, CDCl₃), mixture of diastereomers, δ171.7, 165.9, 165.8, 155.5, 150.5, 150.3, 149.5, 149.3, 136.6, 131.9, 129.9, 129.0, 128.9, 127.6, 125.0, 117.1, 115.3, 115.1, 110.9, 100.4, 96.5, 91.4, 88.3, 71.2, 70.8, 66.5, 66.3, 63.9, 61.0, 56.2, 56.1, 52.0, 41.0, 40.9, 32.5, 32.0, 31.1, 30.9, 25.2, 20.2; MS (ES+): m/z=643 (M+H)⁺; LCMS (Method A): t_{R}=8.67 min.

### Example 118: 2-(3-((((12aS)-11-((Allyloxy)carbonyl)-8-methoxy-6-oxo-12-((tetrahydro-2H-pyran-2-yl)oxy)-11,12,12a,13-tetrahydro-6H-benzo[5,6]-[1,4]diazepino[1,2-a]indol-9-yl)oxy)methyl)phenyl)acetic acid (116)

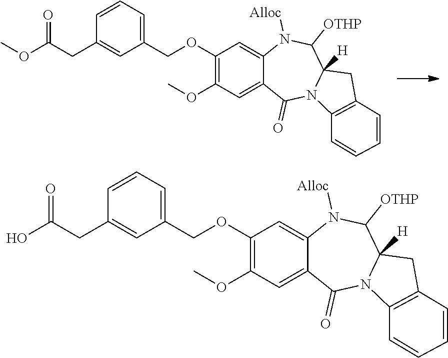

A solution of allyl (12aS)-8-methoxy-9-((3-(2-methoxy-2-oxoethyl)benzyl)oxy)-6-oxo-12-((tetrahydro-2H-pyran-2-yl)oxy)-12a,13-dihydro-6H-benzo[5,6][1,4]diazepino[1,2-a]indole-11(12H)-carboxylate (115) (223 mg, 0.36 mmol) in tetrahydrofuran (4 mL) was charged with an aqueous solution of sodium hydroxide (1 M, 720 μL, 0.72 mmol) and stirred at room temperature, whilst monitoring by TLC and LCMS. Once the reaction was judged to be complete, the mixture was then partially concentrated in vacuo (to remove tetrahydrofuran), then diluted into ethyl acetate and acidified to pH=3-4 with a saturated aqueous solution of citric acid. The organic layer was separated, and the aqueous layer washed with ethyl acetate. The combined organic extracts were then washed with brine, dried over magnesium sulfate, filtered and concentrated in vacuo. The resulting white solid was used in the next step without any further purification.

MS (ES+): m/z=629 (M+H)⁺; LCMS (Method A): t_{R}=7.97 min.

### Example 119: Allyl (12aS)-Q-((3-(2-((S)-1-(chloromethyl)-5-hydroxy-1,2-dihydro-3H-benzo[e]indol-3-yl)-2-oxoethyl)benzyl)oxy)-8-methoxy-6-oxo-12-((tetrahydro-2H-pyran-2-yl)oxy)-12a,13-dihydro-6H-benzo[5,6][1,4]-diazepino[1,2-a]indole-11(12H)-carboxylate (117)

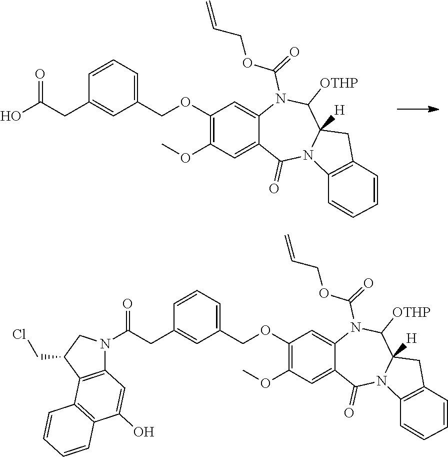

A solution of 2-(3-((((12aS)-11-((allyloxy)carbonyl)-8-methoxy-6-oxo-12-((tetrahydro-2H-pyran-2-yl)oxy)-11,12,12a,13-tetrahydro-6H-benzo[5,6][1,4]diazepino[1,2-a]indol-9-yl)oxy)methyl)phenyl)acetic acid (116) (85 mg, 0.14 mmol) in N,N-dimethylacetamide (1 mL) was charged to (S)-1-(chloromethyl)-2,3-dihydro-1H-benzo[e]indol-5-ol hydrochloride (11) (38 mg, 0.14 mmol), followed byN-(3-dimethylaminopropyl)-N′-ethylcarbodiimide hydrochloride (107 mg, 0.56 mmol) and stirred at room temperature for 16 h. The resulting mixture was diluted into ethyl acetate and washed with cold brine (twice), then dried over magnesium sulfate, filtered and concentrated in vacuo. Purification was enacted by flash column chromatography (silica), eluting with ethyl acetate/petroleum spirit, 40-60° C. (56%), to give the title compound (68 mg, 60%) as a grey solid.

¹H NMR (400 MHz, CDCl₃), mixture of diastereomers, δ10.03 (br, 0.5H), 9.75 (br, 0.5H), 8.40 (d, J=17.5 Hz, 1H), 8.24 (d, J=30.8 Hz, 1H), 8.15 (d, J=70.8 Hz, 1H), 7.62 (d, J=8.3 Hz, 1H), 7.50 (dd, J=12.9, 5.7 Hz, 2H), 7.36 (dd, J=90.6, 5.3 Hz, 3H), 7.27-7.22 (m, 2H), 7.19 (t, J=6.9 Hz, 1H), 7.07 (t, J=7.4 Hz, 1H), 6.95 (s, 0.5H), 6.65 (s, 0.5H), 6.04-5.94 (m, 1H), 5.86 (d, J=9.4 Hz, 1H), 5.66-5.51 (m, 1H), 5.21-5.09 (m, 3H), 5.00 (d, J=13.4 Hz, 2H), 4.52-4.39 (m, 1H), 4.32 (t, J=10.0 Hz, 1H), 4.17-3.99 (m, 3H), 3.98-3.87 (m, 4H), 3.87-3.81 (m, 5H), 3.58-3.51 (m, 1H), 3.51-3.36 (m, 1H), 3.35-3.18 (m, 1H), 1.77-1.30 (m, 6H); MS (ES+): m/z=845 (M+H)⁺; LCMS (Method A): t_{R}=9.00 min.

### Example 120: (S)-Q-((3-(2-((S)-1-(Chloromethyl)-5-hydroxy-1,2-dihydro-3H-benzo[e]indol-1-yl)-2-oxoethyl)benzyl)oxy)-8-methoxy-12a,13-dihydro-6H-benzo[5,6][1,4]diazepino[1,2-a]indol-6-one (118)

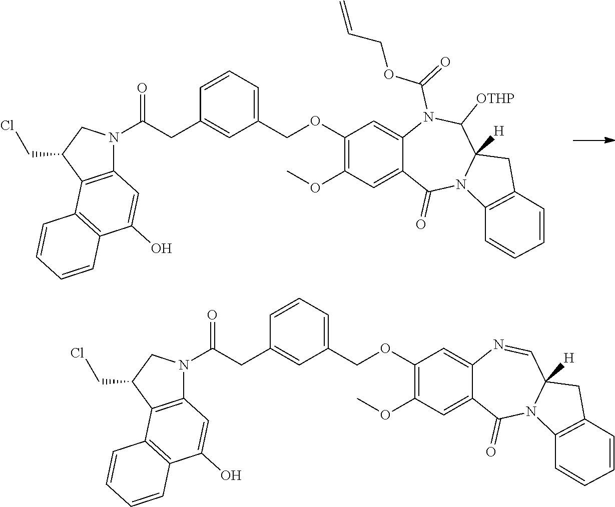

A solution of allyl (12aS)-9-((3-(2-((S)-1-(chloromethyl)-5-hydroxy-1,2-dihydro-3H-benzo [e]indol-3-yl)-2-oxoethyl)benzyl)oxy)-8-methoxy-6-oxo-12-((tetrahydro-2H-pyran-2-yl)oxy)-12a,13-dihydro-6H-benzo[5,6][1,4]diazepino[1,2-a]indole-11(12H)-carboxylate (117) (68 mg, 0.081 mmol) in dichloromethane (1 mL) was charged with pyrrolidine (11 μL, 0.18 mmol), and tetrakis(triphenylphosphine)palladium(0) (9 mg, 0.008 mmol) and stirred at room temperature whilst monitoring by TLC and LCMS. After the reaction was judged to be complete (15 min), the mixture was diluted in dichloromethane and filtered through a pad of celite. The filtrate was concentrated in vacuo, then charged with diethyl ether and concentrated again. Diethyl ether was charged once more, and the residue concentrated in vacuo for a third time. Purification was enacted by flash column chromatography (silica), eluting with ethyl acetate/petroleum spirit, 40-60° C. (from 50% to 100%), to give the title compound (30 mg, 58%) as a pale yellow solid.

¹H NMR (400 MHz, DMSO-d₆) δ 10.31 (s, 1H), 8.07 (d, J=8.0 Hz, 1H), 8.03 (d, J=8.2 Hz, 1H), 7.95 (d, J=4.5 Hz, 1H), 7.91 (s, 1H), 7.72 (d, J=8.2 Hz, 1H), 7.43 (t, J=70.6 Hz, 1H), 7.38-7.32 (m, 4H), 7.30-7.24 (m, 2H), 7.23-7.11 (m, 2H), 7.09-7.01 (m, 1H), 6.92 (s, 1H), 5.20 (d, J=11.9 Hz, 1H), 5.12 (d, J=11.8 Hz, 1H), 5.00 (br, 1H), 4.48 (dt, J=10.1, 5.1 Hz, 1H), 4.32-4.20 (m, 2H), 4.05 (br, 2H), 3.89 (s, 3H), 3.78 (s, 2H), 3.73-3.63 (m, 1H), 3.55-3.50 (m, 1H); MS (ES+): m/z=658 (M+H)⁺; LCMS (Method A): t_{R}=7.82 min.

### Example 121: Methyl (2S,4R)-4-hydroxy-1-(s-methoxy-2-nitro-4-((triisopropylsilyl)oxy)benzoyl)pyrrolidine-2-carboxylate (119)

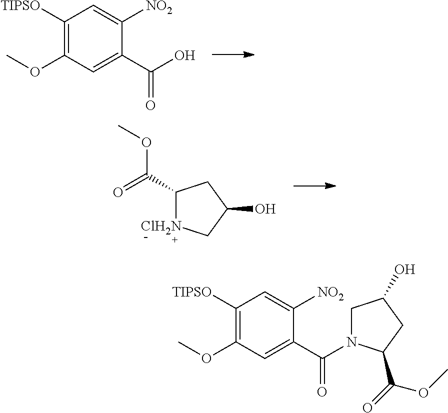

A solution of 5-methoxy-2-nitro-4-((triisopropylsilyl)oxy)benzoic acid (67) (136 g, 0.37 mol) in dichloromethane (1 L) was charged with methyl (2S,4R)-4-hydroxypyrrolidine-2-carboxylate hydrochloride (67.2 g, 0.37 mol), HATU (271 g, 0.74 mol) and N,N-diisopropylethylamine (166.4 g, 1.29 mol) and the resulting mixture stirred for 1 h. Water (2 L) was then added, and dichloromethane (1.5 L). The phases were separated, and the aqueous layer extracted with dichloromethane (1.5 L). The combined organic phases were then dried over sodium sulfate, filtered and concentrated in vacuo. Purification by flash column chromatography (silica), eluting with ethyl acetate/petroleum spirit, 40-60° C. (50%), gave the title compound (114 g, 62%) as a yellow solid.

¹H NMR (400 MHz, CDCl₃) δ 7.68 (s, 1H), 6.82-6.76 (m, 1H), 4.86 (t, J=8.0 Hz, 1H), 4.60-4.43 (m, 1H), 3.92-3.87 (m, 3H), 3.81-3.79 (m, 3H), 3.56-3.48 (m, 2H), 3.19-3.15 (m, 1H), 2.46-2.34 (m, 1H), 2.20-2.14 (m, 1H), 1.32-1.22 (m, 3H), 1.10-1.07 (m, 18H); MS (ES+): m/z=497 (M+H)⁺; LCMS (Method D): t_{R}=2.17 min.

### Example 122: ((2S,4R)-4-Hydroxy-2-(hydroxymethyl)pyrrolidin-1-yl)(5-methoxy-2-nitro-4-((triisopropylsilyl)oxy)phenyl)methanone (120)

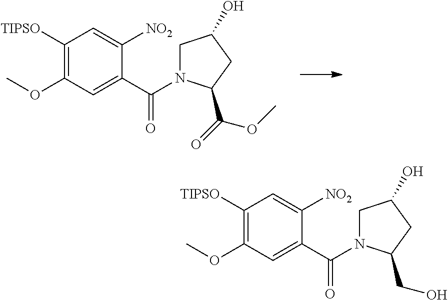

A solution of methyl (2S,4R)-4-hydroxy-1-(5-methoxy-2-nitro-4-((triisopropylsilyl)oxy)benzoyl)pyrrolidine-2-carboxylate (119) (114 g, 0.23 mol) in tetrahydrofuran (1.1 L) was charged slowly with lithium borohydride (2 M in tetrahydrofuran, 460 mL, 0.92 mol) at 0° C. under nitrogen and stirred for 2 h. The reaction was then quenched by cautious addition of water (2 L) and extracted with ethyl acetate (2×1.5 L). The combined organic phases were then dried over magnesium sulfate, filtered and concentrated in vacuo. Purification by flash column chromatography (silica), eluting with ethyl acetate/petroleum spirit, 40-60° C. (67%), gave the title compound (89.2 g, 83%) as a yellow oil.

¹H NMR (400 MHz, CDCl₃) δ 7.68 (s, 1H), 6.82 (s, 1H), 4.60-4.51 (m, 1H), 4.36 (s, 1H), 4.02-3.99 (m, 1H), 3.92 (s, 3H), 3.83-3.72 (m, 1H), 3.35-3.29 (m, 1H), 3.16-3.13 (m, 1H), 2.22-2.17 (m, 1H), 2.00-1.88 (m, 1H), 1.29-1.24 (m, 3H), 1.12-1.05 (m, 18H); MS (ES+): m/z=469 (M+H)⁺; LCMS (Method D): t_{R}=1.75 min.

### Example 123: ((2S,4R)-2-(((tert-Butyldimethylsilyl)oxy)methyl)-4-hydroxypyrrolidin-1-yl)(5-methoxy-2-nitro-4-((triisopropylsilyl)-oxy)phenyl)methanone (121)

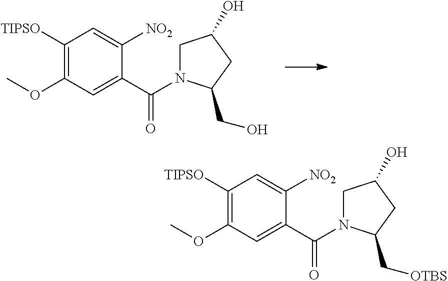

A solution of ((2S,4R)-4-hydroxy-2-(hydroxymethyl)pyrrolidin-1-yl)(5-methoxy-2-nitro-4-((triisopropylsilyl)oxy)phenyl)methanone (120) (75.0 g, 0.16 mol) in dichloromethane (750 mL) was charged with triethylamine (194.5 g, 1.92 mol) and tert-butyldimethylsilyl chloride (169.0 g, 1.12 mol). The reaction mixture was stirred at room temperature for 2 h, then diluted with water (2 L) and extracted with dichloromethane (2×1 L). The combined organic phases were dried over sodium sulfate, filtered and concentrated in vacuo. The residue was purified by flash column chromatography (silica), eluting with ethyl acetate/petroleum spirit, 40-60° C. (50%), to give the title compound (57-7 g, 62%) as a yellow solid.

¹H NMR (400 MHz, CDCl₃) δ 7.70-7.66 (m, 1H), 6.79-6.73 (m, 1H), 4.54 (s, 1H), 4.41 (s, 1H), 3.89-3.87 (m, 5H), 3.79-3.78 (m, 1H), 3.37-3.34 (m, 1H), 3.09-3.07 (m, 1H), 2.35-2.30 (m, 1H), 2.12-2.08 (m, 1H), 1.29-1.27 (m, 3H), 1.10-1.08 (m, 18H), 0.91 (s, 9H), 0.10 (d, J=4.0 Hz, 6H); MS (ES+): m/z=605 (M+Na)⁺; LCMS (Method D): t_{R}=3.90 min.

### Example 124: (S)-5-(((tert-Butyldimethylsilyl)oxy)methyl)-1-(5-methoxy-2-nitro-4-((triisopropylsilyl)oxy)benzoyl)pyrrolidin-3-one (122)

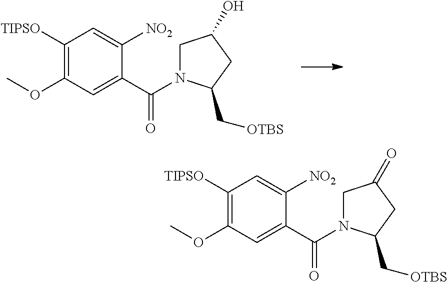

A solution of ((2S,4R)-2-(((tert-butyldimethylsilyl)oxy)methyl)-4-hydroxypyrrolidin-1-yl)(5-methoxy-2-nitro-4-((triisopropylsilyl)oxy)phenyl)methanone (121) (50.0 g, 85.8 mmol) in dichloromethane (500 mL) was charged with 2,2,6,6-tetramethyl-1-piperidinyloxy (1.34 g, 8.60 mmol) and (diacetoxyiodo)benzene (33.2 g, 103.0 mmol) and the resulting mixture stirred at room temperature for 12 h. The mixture was then diluted into water (2 L) and extracted with dichloromethane (1×2 L). The combined organic phases were then dried over sodium sulfate, filtered and concentrated in vacuo.

Purification by flash column chromatography (silica), eluting with ethyl acetate/petroleum spirit, 40-60° C. (25%), gave the title compound (48.8 g, 98%) as a yellow oil.

¹H NMR (400 MHz, CDCl₃) δ 7.72-7.68 (m, 1H), 6.79-6.71 (m, 1H), 4.97 (d, J=70.6 Hz, 1H), 4.28 (d, J=8.0 Hz, 1H), 3.88 (s, 3H), 3.71 (d, J=8.4 Hz, 1H), 3.65-3.61 (m, 1H), 3.44 (d, J=14.0 Hz, 1H), 2.80-2.74 (m, 1H), 2.56-2.52 (m, 1H), 1.26-1.25 (m, 3H), 1.10-1.09 (m, 18H), 0.85 (s, 9H), 0.08 (d, J=10.0 Hz, 6H); MS (ES+): m/z=603 (M+Na)⁺; LCMS (Method D): t_{R}=4.17 min.

### Example 125: (S)-5-(((tert-Butyldimethylsilyl)oxy)methyl)-1-(5-methoxy-2-nitro-4-((triisopropylsilyl)oxy)benzoyl)-4,5-dihydro-1H-pyrrol-3-yl trifluoromethanesulfonate (123)

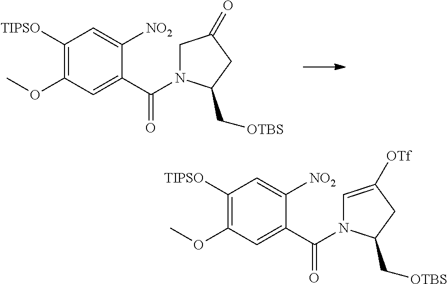

A solution of (S)-5-(((tert-butyldimethylsilyl)oxy)methyl)-1-(5-methoxy-2-nitro-4-((triisopropylsilyl)oxy)benzoyl)pyrrolidin-3-one (122) (30.0 g, 51.7 mmol) in dichloromethane (300 mL) was cooled to −50° C. and charged with 2,6-lutidine (33.2 g, 310.1 mmol) under nitrogen. Triflic anhydride (43.8 g, 155.1 mmol) was then added and the resulting mixture stirred at the same temperature for 1.5 h, after which water (1 L) was added. The mixture was then extracted with dichloromethane (2×500 mL), and the combined organic phases dried over sodium sulfate, filtered and concentrated in vacuo. Purification by flash column chromatography (silica), eluting with ethyl acetate/petroleum spirit, 40-60° C. (10%), gave the title compound (30.0 g, 81%) as a brown oil.

¹H NMR (400 MHz, CDCl₃) δ 7.71 (s, 1H), 6.75 (s, 1H), 6.05 (s, 1H), 4.78 (s, 1H), 3.93-3.88 (m, 5H), 3.21-3.11 (m, 1H), 3.01-2.96 (m, 1H), 1.29-1.27 (m, 3H), 1.11 (s, 9H), 1.10 (s, 9H), 0.91 (s, 9H), 0.11 (d, J=1.6 Hz, 6H); MS (ES+): m/z=735 (M+Na)⁺; LCMS (Method D): t_{R}=2.45 min.

### Example 126: (S)-(2-(((tert-Butyldimethylsilyl)oxy)methyl)-4-methyl-2,3-dihydro-1H-pyrrol-1-yl)(5-methoxy-2-nitro-4-((triisopropylsilyl)oxy)-phenyl)methanone (124)

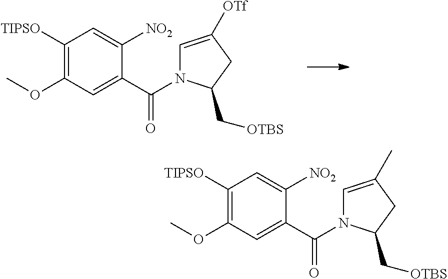

A solution of (S)-5-(((tert-butyldimethylsilyl)oxy)methyl)-1-(5-methoxy-2-nitro-4-((triisopropylsilyl)oxy)benzoyl)-4,5-dihydro-1H-pyrrol-3-yl trifluoromethanesulfonate (123) (30.0 g, 42.1 mmol) in 1,4-dioxane (300 mL) was charged with methylboronic acid (8.82 g, 147.0 mmol), silver (I) oxide (39.0 g, 168.0 mmol), potassium phosphate (54.0 g, 252.0 mmol), triphenylarsine (5.16 g, 16.8 mmol) and bis(benzonitrile)palladium(II) chloride (1.62 g, 4.21 mmol). The reaction mixture was stirred at 110° C. for 10 min under nitrogen. It was then filtered and concentrated in vacuo. The residue was purified by flash column chromatography (silica), eluting with ethyl acetate/petroleum spirit, 40-60° C. (17%), to afford the title compound (15.8 g, 65%) as a brown oil.

¹H NMR (400 MHz, CDCl₃) δ 7.70 (s, 1H), 6.77 (s, 1H), 5.51-5.50 (m, 1H), 4.67-4.65 (m, 1H), 3.92-3.86 (m, 5H), 2.82-2.72 (m, 1H), 2.57-2.52 (m, 1H), 1.62 (d, J=1.2 Hz, 3H), 1.30-1.26 (m, 3H), 1.11 (s, 9H), 1.09 (s, 9H), 0.90 (s, 9H), 0.10 (d, J=2.4 Hz, 6H); MS (ES+): m/z=601 (M+Na)⁺; LCMS (Method D): t_{R}=2.44 min.

### Example 127: (S)-(2-Amino-5-methoxy-4-((triisopropylsilyl)oxy)phenyl)(2-(((tert-butyldimethylsilyl)oxy)methyl)-4-methyl-2,3-dihydro-1H-pyrrol-1-yl)methanone (125)

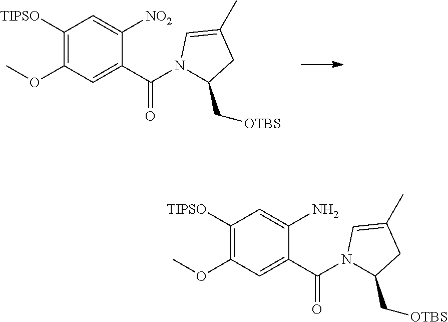

A solution of (S)-(2-(((tert-butyldimethylsilyl)oxy)methyl)-4-methyl-2,3-dihydro-1H-pyrrol-1-yl)(5-methoxy-2-nitro-4-((triisopropylsilyl)oxy)phenyl)methanone (124) (15.0 g, 25.9 mmol) in ethanol (160 mL) and water (40 mL) was charged with iron (7.24 g, 129.7 mmol) and ammonium chloride (6.93 g, 129.7 mmol). The reaction mixture was stirred at 80° C. for 2 h under nitrogen and then filtered and concentrated in vacuo. The residue was purified by flash column chromatography (silica), eluting with ethyl acetate/petroleum spirit, 40-60° C. (10%), to afford the title compound (10.1 g, 76%) as a yellow oil.

¹H NMR (400 MHz, CDCl₃) δ 6.72 (s, 1H), 6.26 (s, 1H), 6.15 (s, 1H), 4.63-4.61 (m, 1H), 3.93-3.87 (m, 1H), 3.78-3.77 (m, 1H), 3.70 (s, 3H), 2.76-2.66 (m, 1H), 2.55-2.50 (m, 1H), 1.67 (s, 3H), 1.25-1.23 (m, 3H), 1.09-1.07 (m, 18H), 0.88 (s, 9H), 0.06-0.04 (m, 6H); MS (ES+): m/z=549 (M+H)⁺; LCMS (Method D): t_{R}=2.56 min.

### Example 128: Allyl (S)-(2-(2-(((tert-butyldimethylsilyl)oxy)methyl)-4-methyl-2,3-dihydro-1H-pyrrole-1-carbonyl)-4-methoxy-5-((triisopropylsilyl)oxy)phenyl)carbamate (126)

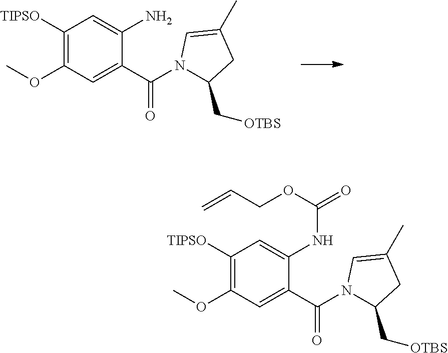

A solution of (S)-(2-amino-5-methoxy-4-((triisopropylsilyl)oxy)phenyl)(2-(((tert-butyldimethylsilyl)oxy)methyl)-4-methyl-2,3-dihydro-1H-pyrrol-1-yl)methanone (125) (10.0 g, 18.2 mmol) in dichloromethane (100 mL) was charged with pyridine (2.88 g, 36.5 mmol). Allyl chloroformate (2.30 g, 19.1 mmol) was then added at −5° C. The reaction mixture was stirred at −5° C. for 30 min and then diluted with water (500 mL) and extracted with dichloromethane (2×300 mL). The combined organic phases were then dried over sodium sulfate, filtered and concentrated in vacuo. The residue was purified by flash column chromatography (silica), eluting with ethyl acetate/petroleum spirit, 40-60° C. (10%), to afford the title compound (10.7 g, 93%) as a yellow oil.

¹H NMR (400 MHz, DMSO-d₆) δ 8.96 (s, 1H), 7.20 (s, 1H), 6.81 (s, 1H), 6.02 (s, 1H), 5.95, 5.86 (m, 1H), 5.31-5.27 (m, 1H), 5.21-5.17 (m, 1H), 4.57-4.42 (m, 3H), 3.83-3.67 (m, 5H), 2.75-2.64 (m, 1H), 2.43-2.35 (m, 1H), 1.62 (s, 3H), 1.25-1.21 (m, 3H), 1.06-1.03 (m, 18H), 0.86 (s, 9H), 0.05-0.03 (m, 6H); MS (ES+): m/z=633 (M+H)⁺; LCMS (Method D): t_{R}=3.57 min.

### Example 129: Allyl (S)-(2-(2-(hydroxymethyl)-4-methyl-2,3-dihydro-1H-pyrrole-1-carbonyl)-4-methoxy-5-((triisopropylsilyl)oxy)phenyl)carbamate (127)

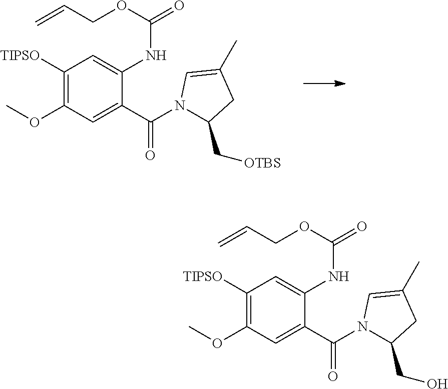

A solution of allyl (S)-(2-(2-(((tert-butyldimethylsilyl)oxy)methyl)-4-methyl-2,3-dihydro-1H-pyrrole-1-carbonyl)-4-methoxy-5-((triisopropylsilyl)oxy)phenyl)carbamate (126) (9.70 g, 15.3 mmol) in acetic acid/methanol/tetrahydrofuran/water (7:1:1:2, 110 mL) was stirred at room temperature for 2 h. The reaction mixture was diluted with a saturated aqueous solution of sodium hydrogen carbonate (400 mL) and extracted with ethyl acetate (2×300 mL). The combined organic phases were then dried over sodium sulfate, filtered and concentrated in vacuo. The residue was purified by flash column chromatography (silica), eluting with ethyl acetate/petroleum spirit, 40-60° C. (25%), to afford the title compound (7.60 g, 95%) as a colourless oil.

¹H NMR (400 MHz, DMSO-d₆) δ 8.93 (s, 1H), 7.18 (s, 1H), 6.87 (s, 1H), 5.95-5.86 (m, 2H), 5.34-5.26 (m, 1H), 5.21-5.17 (m, 1H), 4.84-4.81 (m, 1H), 4.55-4.39 (m, 3H), 3.74 (s, 3H), 3.69-3.60 (m, 1H), 3.56-3.48 (m, 1H), 2.73-2.62 (m, 1H), 2.45-2.37 (m, 1H), 1.62 (s, 3H), 1.25-1.22 (m, 3H), 1.06-1.04 (m, 18H); MS (ES+): m/z=519 (M+H)⁺; LCMS (Method D): t_{R}=2.67 min.

### Example 130: Allyl (11aS)-11-hydroxy-7-methoxy-2-methyl-5-oxo-8-((triisopropylsilyl)oxy)-11,11a-dihydro-1H-benzo[e]pyrrolo[1,2-a][1,4]diazepine-10(5H)-carboxylate (128)

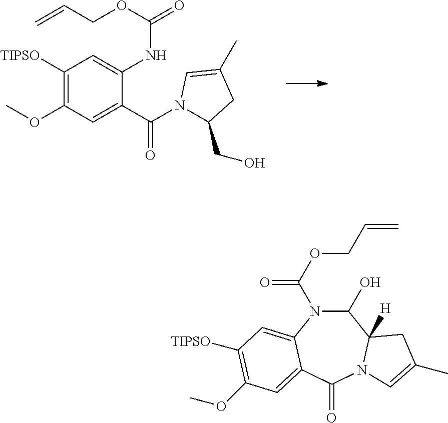

A solution of allyl (S)-(2-(2-(hydroxymethyl)-4-methyl-2,3-dihydro-1H-pyrrole-1-carbonyl)-4-methoxy-5-((triisopropylsilyl)oxy)phenyl)carbamate (127) (7.60 g, 14.7 mmol) in dichloromethane (80 mL) was charged with 2,2,6,6-tetramethyl-1-piperidinyloxy (0.23 g, 1.47 mmol) and (diacetoxyiodo)benzene (5.19 g, 16.1 mmol). The reaction mixture was stirred at room temperature for 12 h and then diluted with water (300 mL) and extracted with dichloromethane (2×300 mL). The combined organic phases were then dried over sodium sulfate, filtered and concentrated in vacuo. The residue was purified by flash column chromatography (silica), eluting with ethyl acetate/petroleum spirit, 40-60° C. (25%) to afford the title compound (3.90 g, 51%) as a light-green oil.

¹H NMR (400 MHz, CDCl₃) δ 7.19 (s, 1H), 6.72-6.67 (m, 2H), 5.81-5.71 (m, 2H), 5.23-5.08 (m, 2H), 4.61-4.56 (m, 1H), 4.49-4.42 (m, 1H), 3.84 (s, 3H), 3.66-3.55 (m, 1H), 3.02-2.90 (m, 1H), 2.63-2.54 (m, 1H), 2.29-2.21 (m, 1H), 1.79-1.75 (m, 3H), 1.22-1.20 (m, 3H), 1.08-1.06 (m, 18H); MS (ES+): m/z=517 (M+H)⁺; LCMS (Method D): t_{R}=2.09 min.

### Example 131: Allyl (11aS)-7-methoxy-2-methyl-5-oxo-11-((tetrahydro-2H-pyran-2-yl)oxy)-8-((triisopropylsilyl)oxy)-11,11a-dihydro-1H-benzo[e]pyrrolo[1,2-a][1,4]diazepine-10(5H)-carboxylate (129)

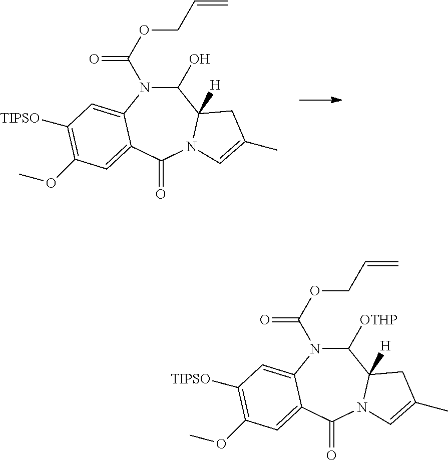

A solution of allyl (11aS)-11-hydroxy-7-methoxy-2-methyl-5-oxo-8-((triisopropylsilyl)oxy)-11,11a-dihydro-1H-benzo[e]pyrrolo[1,2-a][1,4]diazepine-10(5H)-carboxylate (128) (3.90 g, 7.55 mmol) in tetrahydrofuran (50 mL) was charged with 3,4-dihydro-2H-pyran (6.35 g, 75.5 mmol) and p-toluenesulfonic acid (0.13 g, 0.75 mmol). The reaction mixture was stirred at room temperature for 12 h and then diluted into water (300 mL) and extracted with ethyl acetate (2×300 mL). The combined organic phases were then dried over sodium sulfate, filtered and concentrated in vacuo. The residue was purified by flash column chromatography (silica), eluting with ethyl acetate/petroleum spirit, 40-60° C. (20%) to afford the title compound (4.30 g, 95%) as a colourless oil.

¹H NMR (400 MHz, CDCl₃) δ 7.19-7.16 (m, 1H), 6.93-6.57 (m, 2H), 6.01-5.86 (m, 1H), 5.75-5.71 (m, 1H), 5.20-4.97 (m, 3H), 4.62-4.52 (m, 1H), 4.47-4.29 (m, 1H), 3.96-3.88 (m, 1H), 3.84 (d, J=2.0 Hz, 3H), 3.80-3.73 (m, 1H), 3.65-3.50 (m, 1H), 2.99-2.89 (m, 1H), 2.65-2.41 (m, 1H), 1.80-1.71 (m, 5H), 1.58-1.46 (m, 4H), 1.25-1.17 (m, 3H), 1.10-1.05 (m, 18H); MS (ES+): m/z=601 (M+H)⁺; LCMS (Method D): t_{R}=3.90 min.

### Example 132: Allyl (11aS)-8-hydroxy-7-methoxy-2-methyl-5-oxo-11-((tetrahydro-2H-pyran-2-yl)oxy)-11,11a-dihydro-1H-benzo[e]pyrrolo[1,2-a][1,4]diazepine-10(5H)-carboxylate (130)

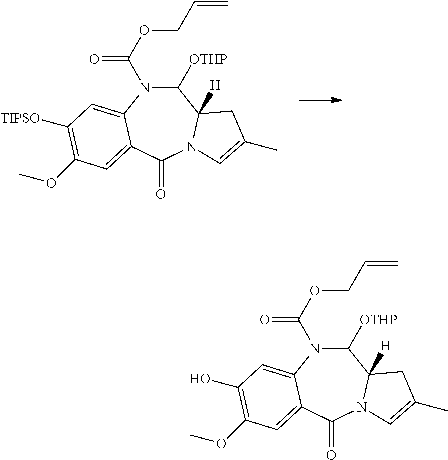

A solution of allyl (11aS)-7-methoxy-2-methyl-5-oxo-11-((tetrahydro-2H-pyran-2-yl)oxy)-8-((triisopropylsilyl)oxy)-11,11a-dihydro-1H-benzo [e]pyrrolo [1,2-a][1,4]diazepine-10(5H)-carboxylate (129) (4.30 g, 7.16 mmol) in tetrahydrofuran (35 mL) was charged with tetrabutylammonium fluoride (1 M in tetrahydrofuran, 9.3 mL, 9.31 mmol). The reaction mixture was stirred at room temperature for 1 h under nitrogen and then diluted into water (200 mL) and extracted with ethyl acetate (2×200 mL). The combined organic phases were dried over sodium sulfate, filtered and concentrated in vacuo. The residue was then purified by flash column chromatography (silica), eluting with ethyl acetate/petroleum spirit, 40-60° C. (50%) to afford the title compound (2.50 g, 78%) as a white solid.

¹H NMR (400 MHz, CDCl₃) δ 7.23-7.20 (m, 1H), 6.99-6.66 (m, 2H), 6.07-5.86 (m, 2H), 5.83-5.68 (m, 1H), 5.19-4.98 (m, 3H), 4.68-4.54 (m, 1H), 4.50-4.35 (m, 1H), 3.97-3.89 (m, 4H), 3.85-3.75 (m, 1H), 3.65-3.53 (m, 1H), 2.99-2.90 (m, 1H), 2.70-2.40 (m, 1H), 1.80-1.72 (m, 5H), 1.58-1.49 (m, 4H); MS (ES+): m/z=445 (M+H)⁺; LCMS (Method D): t_{R}=1.00 min.

### Example 133: Allyl (11aS)-7-methoxy-8-((3-(2-methoxy-2-oxoethyl)benzyl)-oxy)-2-methyl-5-oxo-11-((tetrahydro-2H-pyran-2-yl)oxy)-11,1a-dihydro-1H-benzo[e]pyrrolo[1,2-a][1,4]diazepine-10(5H)-carboxylate (131)

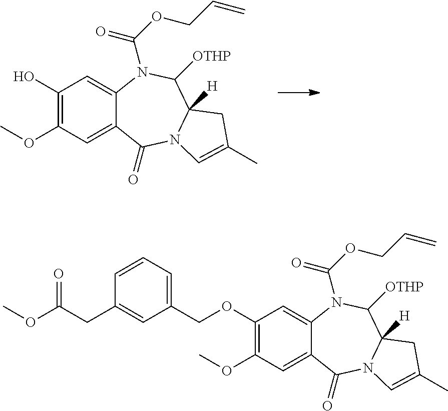

A solution of allyl (11aS)-8-hydroxy-7-methoxy-2-methyl-5-oxo-11-((tetrahydro-2H-pyran-2-yl)oxy)-11,11a-dihydro-1H-benzo[e]pyrrolo[1,2-a][1,4]diazepine-10(5H)-carboxylate (130) (444 mg, 0.999 mmol) in N,N-dimethylformamide (2 mL) was charged with potassium carbonate (207 mg, 1.50 mmol) and 3-(bromomethyl)-benzeneacetic acid methyl ester (466 mg, 1.92 mmol) and stirred at room temperature overnight. The reaction mixture was then diluted into ethyl acetate (200 mL) and washed with cold brine (2×100 mL). The organic phase was then dried over magnesium sulfate, filtered and concentrated in vacuo. Purification by flash column chromatography (silica), eluting with ethyl acetate/petroleum spirit, 40-60° C. (from 0% to 100%) gave the title compound (452 mg, 75%) as a white solid.

¹H NMR (400 MHz, CDCl₃), mixture of diastereomers, δ 7.32-7.30 (m, 2H), 7.30 (d, J=1.9 Hz, 1H), 7.24 (d, J=2.5 Hz, 1H), 7.23-7.19 (m, 1H), 6.91 (s, 1H), 6.66 (s, 1H), 5.98 (d, J=9.5 Hz, 1H), 5.86 (d, J=9.3 Hz, 1H), 5.75-5.57 (m, 1H), 5.15-4.95 (m, 4H), 4.52 (d, J=13.5 Hz, 1H), 4.33 (d, J=14.1 Hz, 1H), 3.89 (s, 3H), 3.88-3.72 (m, 2H), 3.66 (s, 3H), 3.61 (s, 2H), 3.48-3.40 (m, 1H), 2.99-2.86 (m, 1H), 2.61 (d, J=15.8 Hz, 0.5H), 2.43 (d, J=16.9 Hz, 0.5H), 1.74 (s, 3H), 1.73-1.68 (m, 1H), 1.56-1.46 (m, 4H), 1.39-1.30 (m, 1H); ¹³C NMR (100 MHz, CDCl₃), mixture of diastereomers, δ 171.7, 163.2, 163.1, 149.4, 149.2, 136.7, 136.6, 134.4, 134.3, 132.2, 132.1, 131.9, 129.0, 128.9, 128.8, 128.1, 126.2, 126.1, 123.5, 123.3, 121.1, 116.9, 115.4, 115.2, 110.6, 100.2, 92.2, 70.8, 66.3, 63.8, 63.6, 59.5, 56.2, 56.1, 52.0, 41.0, 39.3, 38.8, 31.0, 25.2, 20.0, 19.9, 13.7, 13.6; MS (ES+): m/z=607 (M+H)⁺; LCMS (Method A): t_{R}=8.18 min.

### Example 134: 2-(3-((((11aS)-10-((Allyloxy)carbonyl)-7-methoxy-2-methyl-5-oxo-11-((tetrahydro-2H-pyran-2-yl)oxy)-5,10,11,11a-tetrahydro-1H-benzo-[e]pyrrolo[1,2-a][1,4]diazepin-8-yloxy)methyl)phenyl)acetic acid (132)

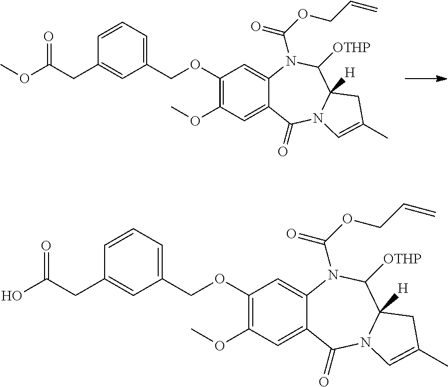

A solution of allyl (11aS)-7-methoxy-8-((3-(2-methoxy-2-oxoethyl)benzyl)oxy)-2-methyl-5-oxo-11-((tetrahydro-2H-pyran-2-yl)oxy)-11,11a-dihydro-1H-benzo[e]pyrrolo[1,2-a][1,4]diazepine-10(5H)-carboxylate (131) (450 mg, 0.74 mmol) in tetrahydrofuran (18 mL) was charged with an aqueous solution of sodium hydroxide (1 M, 4 mL) and stirred at room temperature for 4 h. A saturated aqueous solution of citric acid was then added until pH=4 and the resulting mixture was extracted with ethyl acetate (2×100 mL). The combined organic phases were then dried over magnesium sulfate, filtered and concentrated in vacuo. The resulting residue was then used in the subsequent step without further purification.

MS (ES+): m/z=593 (M+H)⁺; LCMS (Method A): t_{R}=7.42 min.

### Example 135: Allyl (11aS)-8-((3-(2-((S)-1-(chloromethyl)-5-hydroxy-1,2-dihydro-3H-benzo[e]indol-3-yl)-2-oxoethyl)benzyl)oxy)-7-methoxy-2-methyl-5-oxo-11-((tetrahydro-2H-pyran-2-yl)oxy)-11,11a-dihydro-1H-benzo[e]pyrrolo[1,2-a][1,4]diazepine-10(5H)-carboxylate (133)

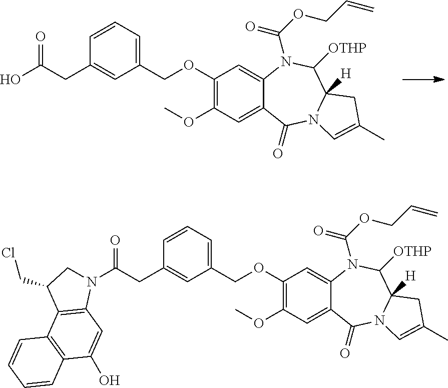

A solution of 2-(3-((((11aS)-10-((allyloxy)carbonyl)-7-methoxy-2-methyl-5-oxo-11-((tetrahydro-2H-pyran-2-yl)oxy)-5,10,11,11a-tetrahydro-1H-benzo[e]pyrrolo[1,2-a][1,4]diazepin-8-yl)oxy)methyl)phenyl)acetic acid (132) (439 mg, 0.74 mmol) in N,N-dimethylacetamide (3 mL) was charged to (S)-1-(chloromethyl)-2,3-dihydro-1H-benzo[e]indol-5-ol hydrochloride (11) (200 mg, 0.74 mmol), followed immediately by N-(3-dimethylaminopropyl)-N′-ethylcarbodiimide hydrochloride (567 mg, 2.96 mmol) and the resulting mixture stirred at room temperature for 16 h. The reaction mixture was subsequently diluted into ethyl acetate (100 mL) and washed with cold brine (2×20 mL), dried over magnesium sulfate, filtered and concentrated in vacuo. Flash column chromatography (silica), eluting with ethyl acetate/petroleum spirit, 40-60° C. (from 50% to 100%), gave the title compound (369 mg, 62%) as a grey solid.

¹H NMR (400 MHz, DMSO-d₆), mixture of diastereomers, δ10.31 (s, 1H), 8.03 (d, J=8.3 Hz, 1H), 7.91 (s, 1H), 7.72 (d, J=8.3 Hz, 1H), 7.43 (t, J=70.6 Hz, 1H), 7.39-7.23 (m, 5H), 7.04 (s, 2H), 7.04 (s, 2H), 6.95 (s, 0.5H), 6.87 (s, 0.5H), 6.58 (s, 1H), 5.74 (dd, J=19.2, 13.1 Hz, 2H), 5.13-4.87 (m, 4H), 4.43 (br, 1H), 4.27 (dt, J=19.7, 12.8 Hz, 2H), 4.07 (br, 1H), 3.88 (d, J=30.6 Hz, 2H), 3.77-3.73 (m, 4H), 3.69 (dt, J=10.9, 6.9 Hz, 2H), 3.42 (dd, J=19.8, 13.3 Hz, 1H), 2.95-2.80 (m, 1H), 2.50 (d, J=15.5 Hz, 0.5H), 2.40 (d, J=19.8 Hz, 0.5H), 1.69 (s, 3H), 1.64-1.23 (m, 6H); MS (ES+): m/z=809 (M+H)⁺; LCMS (Method A): t_{R}=8.77 min.

### Example 136: (S)-8-((I-(2-((S)-1-(Chloromethyl)-5-hydroxy-1,2-dihydro-3H-benzo[e]indol-3-yl)-2-oxoethyl)benzyl)oxy)-7-methoxy-2-methyl-1,11a-dihydro-5H-benzo[e]pyrrolo[1,2-a][1,4]diazepin-5-one (134)

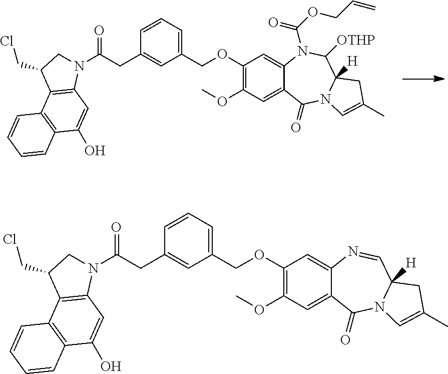

A solution of allyl (11aS)-8-((3-(2-((S)-1-(chloromethyl)-5-hydroxy-1,2-dihydro-3H-benzo [e]indol-3-yl)-2-oxoethyl)benzyl)oxy)-7-methoxy-2-methyl-5-oxo-11-((tetrahydro-2H-pyran-2-yl)oxy)-11,11a-dihydro-1H-benzo[e]pyrrolo[1,2-a][1,4]diazepine-10(5H)-carboxylate (133) (100 mg, 0.12 mmol) in dichloromethane (2 mL) was charged with pyrrolidine (22 μL, 0.264 mmol) and tetrakis(triphenylphosphine)palladium(0) (14 mg, 0.012 mmol) and the resulting mixture stirred for 10 min, after which it was diluted into dichloromethane (10 mL), filtered through a plug of celite. The filter cake was then washed with dichloromethane and the filtrate concentrated in vacuo. Diethyl ether (50 mL) was then charged and the mixture concentrated again. Purification by flash column chromatography (silica), eluting with ethyl acetate/petroleum spirit, 40-60° C. (from 50% to 100%), gave the title compound (45 mg, 58%) as a yellow solid.

¹H NMR (400 MHz, CDCl₃) δ 10-31 (s, 1H), 8.03 (d, J=8.4 Hz, 1H), 7.90 (s, 1H), 7.83 (d, J=4.0 Hz, 1H), 7.72 (d, J=8.2 Hz, 1H), 7.61-7.53 (m, 1H), 7.53-7.47 (m, 1H), 7.44 (t, J=7.5 Hz, 1H), 7.38-7.22 (m, 5H), 6.87 (s, 1H), 6.66 (s, 1H), 5.18-5.06 (m, 1H), 4.97 (d, J=5.8 Hz, 1H), 4.34-4.16 (m, 2H), 4.08 (br, 1H), 3.88 (s, 3H), 3.77-3.65 (m, 2H), 3.64-3.61 (m, 1H), 3.59-3.57 (m, 1H), 3.33 (q, J=7.0 Hz, 1H), 2.95 (br, 1H), 1.69 (s, 3H); MS (ES+): m/z=623 (M+H)⁺; LCMS (Method A): t_{R}=7.37 min.

### Example 137: Methyl (S)-1-(5-methoxy-2-nitro-4-((triisopropylsilyl)oxy)-benzoyl)piperidine-2-carboxylate (135)

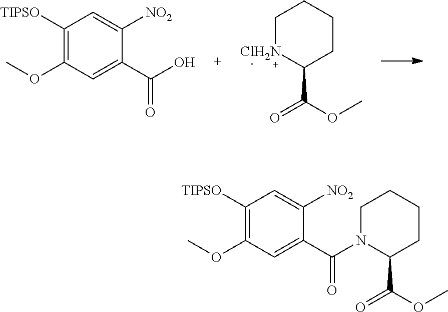

A solution of 5-methoxy-2-nitro-4-((triisopropylsilyl)oxy)benzoic acid (67) (2.00 g, 5.41 mmol) in dichloromethane (12 mL) was charged with triethylamine (3.2 mL, 22.9 mmol) and HATU (2.16 g, 5.68 mmol) and stirred for 5 min at room temperature before methyl (2S)-piperidinecarboxylate hydrochloride (972 mg, 5.41 mmol) was added. The resulting mixture was stirred for 16 h before diluting into ethyl acetate (200 mL) and washing with a saturated aqueous solution of sodium hydrogen carbonate (100 mL×2), followed by an aqueous solution of acetic acid (1% v/v, 100 mL) and brine (100 mL). After drying over magnesium sulfate, filtering and concentrating in vacuo, the residue was purified by flash column chromatography (silica), eluting with ethyl acetate/petroleum spirit, 40-60° C. (from 10% to 50%) to give the title compound (1.27 g, 95%) as a yellow solid.

¹H NMR (400 MHz, CDCl₃), mixture of rotamers, δ7.66 (s, 0.7H), 7.64 (s, 0.3H), 6.77 (s, 0.3H), 6.76 (s, 0.5H), 5.60 (d, J=4.7 Hz, 0.5H), 4.76 (d, J=12.8 Hz, 0.3H), 3.89 (s, 3H), 3.81 (s, 1.3H), 3.78 (s, 1.7H), 3.71-3.69 (m, 1H), 3.28-3.13 (m, 1-5H), 2.84 (td, J=13.1, 3.5 Hz, 0.3H), 2.30 (d, J=12.6 Hz, 0.7H), 1.93-1.82 (m, 0.7H), 1.80-1.63 (m, 2.4H), 1.34-1.17 (m, 4.8H), 1.07 (dd, J=6.5, 5.0 Hz, 18H); ¹³C NMR (100 MHz, CDCl₃), mixture of rotamers, δ 171.6, 171.1, 167.6, 167.0, 156.3, 156.0, 145.7, 137.5, 137.3, 127.2, 126.9, 116.0, 115.9, 109.6, 109.5, 60.3, 58.2, 56.1, 56.0, 52.4, 52.3, 51.9, 45.4, 39.6, 30.9, 27.1, 26.1, 25.0, 24.1, 21.4, 21.2, 17.8, 17.7; MS (ES+): m/z=495 (M+H)⁺; LCMS (Method A): t_{R}=9.32 min.

### Example 138: (S)-3-Hydroxy-2-methoxy-7,8,9,10-tetrahydrobenzo[e]-pyrido[1,2-a][1,4]diazepine-6,12(5H,6aH)-dione (136)

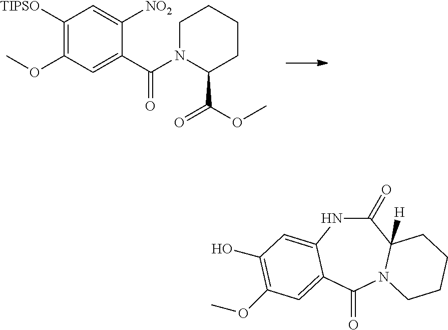

A solution of methyl (S)-1-(5-methoxy-2-nitro-4-((triisopropylsilyl)oxy)benzoyl)-piperidine-2-carboxylate (135) (1.20 g, 2.43 mmol) in tetrahydrofuran (10 mL) was charged with ammonium formate (1.25 g, 19.9 mmol), followed by palladium on activated charcoal (10 wt. % basis, 125 mg) and water (2 mL). The resulting mixture was heated to 65° C., under argon, for 16 h. When the reaction was judged to be complete by TLC and LCMS, it was diluted into ethyl acetate (200 mL) and filtered over celite. The filter cake was washed with ethyl acetate (100 mL) and water (100 mL) and the filtrate phases separated. The organic phase was dried over magnesium sulfate, filtered and concentrated in vacuo. The aqueous phase was frozen and lyophilised to dryness, then charged with ethyl acetate (100 mL), sonicated for 10 min, filtered and the filtrate concentrated in vacuo. The combined red residues were purified by flash column chromatography (silica), eluting with ethyl acetate/petroleum spirit, 40-60° C. (from 10% to 100%), to give the title compound (268 mg, 40%) as a cream solid.

¹H NMR (400 MHz, MeOD) δ 7.26 (s, 1H), 6.51 (s, 1H), 4.41 (dt, J=13.2, 3.6 Hz, 1H), 4.20 (dd, J=6.3, 3.5 Hz, 1H), 3.87 (s, 3H), 2.93 (ddd, J=13.5, 12.1, 3.9 Hz, 1H), 2.23-2.12 (m, 1H), 1.99-1.86 (m, 1H), 1.86-1.76 (m, 1H), 1.75-1.63 (m, 2H), 1.62-1.55 (m, 1H); ¹³C NMR (100 MHz, MeOD) δ 171.6, 169.2, 150.7, 145.2, 131.6, 117.7, 111.9, 106.8, 55.2, 51.5, 39.9, 22.9, 22.1, 18.7; MS (ES+): m/z=277 (M+H)⁺; LCMS (Method A): t_{R}=4.87 min.

### Example 139: Methyl (S)-2-(3-(((2-methoxy-6,12-dioxo-5,6,6a,7,8,9,10,12-octahydrobenzo[e]pyrido[1,2-a][1,4]diazepin-3-yl)oxy)methyl)phenyl)-acetate (137)

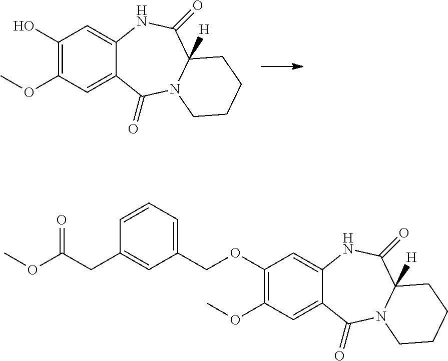 [SMILES: COc1cc2c(cc1O)NC(=O)[C@@H]1CCCCN1C2=O ; COC(=O)Cc1cccc(COc2cc3c(cc2OC)C(=O)N2CCCC[C@H]2C(=O)N3)c1]

A solution of (S)-3-hydroxy-2-methoxy-7,8,9,10-tetrahydrobenzo[e]pyrido[1,2-a][1,4]diazepine-6,12(5H,6aH)-dione (136) (167 mg, 0.604 mmol) in N,N-dimethylformamide (1.2 mL) was charged with potassium carbonate (100 mg, 0.725 mmol) and 3-(bromomethyl)-benzeneacetic acid methyl ester (147 mg, 0.604 mmol). The resulting mixture was stirred for 4 h, before it was diluted into ethyl acetate (100 mL) and washed with cold brine (2×50 mL). The organic phase was dried over magnesium sulfate, filtered and concentrated in vacuo. Purification by flash column chromatography (silica), eluting with ethyl acetate/petroleum spirit, 40-60° C. (from 0% to 100%), gave the title compound (197 mg, 47%) as a white solid.

¹H NMR (400 MHz, CDCl₃) δ 8.80 (s, 1H), 7.33 (s, 1H), 7.31-7.25 (m, 3H), 7.19-7.14 (m, 1H), 6.50 (s, 1H), 5.02 (s, 2H), 4.45 (dt, J=13.6, 3.8 Hz, 1H), 4.11-4.06 (m, 1H), 3.85 (s, 3H), 3.65 (s, 3H), 3.59 (s, 2H), 2.92 (td, J=13.5, 3.9 Hz, 1H), 2.17 (dt, J=14.9, 7.1 Hz, 1H), 1.97-1.82 (m, 1H), 1.82-1.71 (m, 1H), 1.71-1.60 (m, 2H), 1.61-1.47 (m, 1H); ¹³C NMR (100 MHz, CDCl₃) δ 172.1, 171.5, 168.2, 151.1, 146.8, 136.2, 134.3, 130.3, 129.2, 129.0, 128.7, 126.3, 119.7, 112.6, 105.6, 70.8, 56.2, 52.2, 51.3, 40.8, 40.2, 23.1, 22.7, 19.1; MS (ES+): m/z=439 (M+H)⁺; LCMS (Method A): t_{R}=6.30 min.

### Example 140: (S)-2-(3-(((2-Methoxy-6,12-dioxo-5,6,6a,7,8,9,10,12-octa-hydrobenzo[e]pyrido[1,2-a][1,4]diazepin-3-yl)oxy)methyl)phenyl)acetic acid (138)

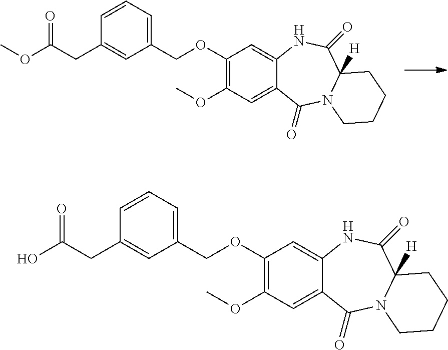 [SMILES: COC(=O)Cc1cccc(COc2cc3c(cc2OC)C(=O)N2CCCC[C@H]2C(=O)N3)c1 ; COc1cc2c(cc1OCc1cccc(CC(=O)O)c1)NC(=O)[C@@H]1CCCCN1C2=O]

A solution of methyl (S)-2-(3-(((2-methoxy-6,12-dioxo-5,6,6a,7,8,9,10,12-octahydrobenzo[e]pyrido[1,2-a][1,4]diazepin-3-yl)oxy)methyl)phenyl)acetate (137) (115 mg, 0.262 mmol) in tetrahydrofuran (1 mL) was charged with an aqueous solution of sodium hydroxide (0.5 M, 1 mL, 0.525 mmol) and the resulting mixture stirred at room temperature for 1.5 h. After the reaction was judged to be complete by TLC and LCMS, it was adjusted to pH=4 with a saturated aqueous solution of citric acid and extracted with ethyl acetate (2×100 mL). The combined organic extracts were then dried over magnesium sulfate, filtered and concentrated in vacuo. The resulting white solid was employed in the subsequent step without further purification. MS (ES+): m/z=425 (M+H)⁺; LCMS (Method A): t_{R}=5.80 min.

### Example 141: (S)-3-((3-(2-((S)-1-(Chloromethyl)-5-hydroxy-1,2-dihydro-3H-benzo[e]indol-3-yl)-2-oxoethyl)benzyl)oxy)-2-methoxy-7,8,9,10-tetrahydrobenzo[e]pyrido[1,2-a][1,4]diazepine-6,12(5H,6aH)-dione (139)

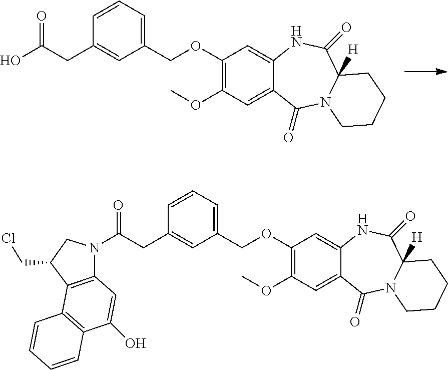 [SMILES: COc1cc2c(cc1OCc1cccc(CC(=O)O)c1)NC(=O)[C@@H]1CCCCN1C2=O ; COc1cc2c(cc1OCc1cccc(CC(=O)N3C[C@@H](CCl)c4c3cc(O)c3ccccc43)c1)NC(=O)[C@@H]1CCCCN1C2=O]

A solution of (S)-2-(3-(((2-Methoxy-6,12-dioxo-5,6,6a,7,8,9,10,12-octahydrobenzo[e]pyrido[1,2-a][1,4]diazepin-3-yl)oxy)methyl)phenyl)acetic acid (138) (111 mg, 0.262 mmol) in N,N-dimethylacetamide (1 mL) was charged to (S)-1-(chloromethyl)-2,3-dihydro-1H-benzo[e]indol-5-ol hydrochloride (11) (127 mg, 0.472 mmol), followed by N-(3-dimethylaminopropyl)-N′-ethylcarbodiimide hydrochloride (271 mg, 1.42 mmol) and the resulting mixture stirred at room temperature for 16 h. The reaction mixture was subsequently diluted into ethyl acetate (100 mL) and washed with cold brine (2×20 mL), dried over magnesium sulfate, filtered and concentrated in vacuo. Flash column chromatography (silica), eluting with ethyl acetate/petroleum spirit, 40-60° C. (90%), followed by trituration in dichloromethane/diethyl ether afforded the title compound (71 mg, 43%) as a cream solid.

¹H NMR (400 MHz, CDCl₃) δ 8.97 (s, 1H), 8.87 (s, 1H), 8.41 (s, 1H), 8.26 (d, J=8.2 Hz, 1H), 7.53 (d, J=8.3 Hz, 1H), 7.47-7.39 (m, 2H), 7.39-7.31 (m, 3H), 7.19 (dd, J=9.7, 5.4 Hz, 1H), 6.91 (d, J=70.6 Hz, 1H), 6.65 (s, 1H), 5.45 (d, J=13.1 Hz, 1H), 5.07 (d, J=13.1 Hz, 1H), 4.45 (dd, J=9.8, 3.7 Hz, 1H), 4.22 (d, J=10.5 Hz, 1H), 4.18-4.12 (m, 1H), 4.11-3.90 (m, 4H), 3.87 (s, 3H), 3.83 (d, J=2.8 Hz, 1H), 3.43 (d, J=16.8 Hz, 1H), 2.92 (td, J=13.5, 3.8 Hz, 1H), 2.25 (d, J=13.0 Hz, 1H), 1.91-1.48 (m, 5H); ¹³C NMR (100 MHz, CDCl₃) δ 171.3, 169.1, 168.0, 155.1, 150.0, 147.4, 141.2, 135.5, 133.4, 132.7, 129.9, 129.6, 129.5, 129.3, 126.5, 123.7, 123.4, 122.4, 122.0, 119.9, 114.4, 112.2, 108.4, 100.5, 71.5, 64.3, 56.2, 53.3, 51.2, 46.5, 42.2, 40.3, 23.2, 22.8, 19.2; MS (ES+): m/z=640 (M+H)⁺; LCMS (Method A): t_{R}=7.25 min.

### Example 142: (S)-1-(Chloromethyl)-3-(2-(3-((((S)-2-methoxy-6,12-dioxo-5,6,6a,7,8,9,10,12-octahydrobenzo[e]pyrido[1,2-a][1,4]diazepin-1-yl)oxy)methyl)phenyl)acetyl)-2,3-dihydro-1H-benzo[e]indol-5-yl 4-methylpiperazine-1-carboxylate (140)

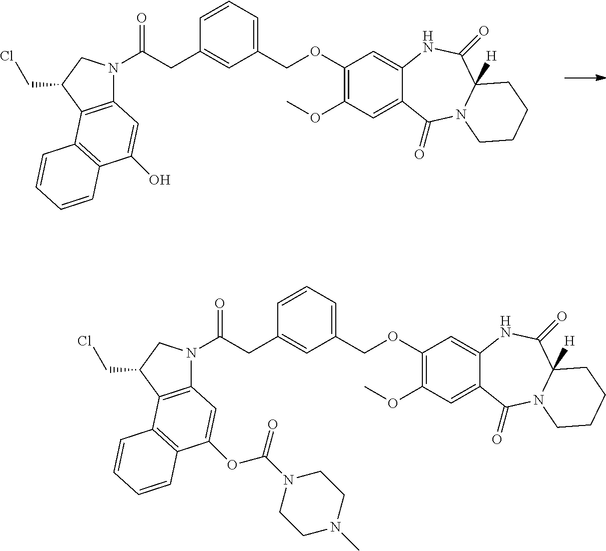 [SMILES: COc1cc2c(cc1OCc1cccc(CC(=O)N3C[C@@H](CCl)c4c3cc(O)c3ccccc43)c1)NC(=O)[C@@H]1CCCCN1C2=O ; COc1cc2c(cc1OCc1cccc(CC(=O)N3C[C@@H](CCl)c4c3cc(OC(=O)N3CCN(C)CC3)c3ccccc43)c1)NC(=O)[C@@H]1CCCCN1C2=O]

A solution of (S)-3-((3-(2-((S)-1-(chloromethyl)-5-hydroxy-1,2-dihydro-3H-benzo [e]indol-3-yl)-2-oxoethyl)benzyl)oxy)-2-methoxy-7,8,9,10-tetrahydrobenzo-[e]pyrido[1,2-a][1,4]diazepine-6,12(5H,6aH)-dione (139) (40 mg, 0.062 mmol) in dichloromethane (1 mL) was charged with 4-methyl-1-piperazinecarbonyl chloride hydrochloride (12 mg, 0.062 mmol), 4-(dimethylamino)pyridine (8.4 mg, 0.069 mmol) and then triethylamine (30 μL, 0.22 mmol). The resulting mixture was stirred at room temperature for 1 h, after which it was concentrated in vacuo, then charged with diethyl ether and concentrated again, then charged with diethyl ether once more and concentrated once again, to give a white solid residue, which was purified by flash column chromatography (silica), eluting with ethyl acetate (100%), then triethylamine/ethyl acetate (5%), and finally with triethylamine/methanol/ethyl acetate (5:5:90) to give the title compound (20 mg, 42%) as a cream solid.

¹H NMR (400 MHz, CDCl₃) δ 8.35 (s, 1H), 8.25 (br, 1H), 7.84 (d, J=8.4 Hz, 1H), 7.66 (d, J=8.4 Hz, 1H), 7.47 (t, J=7.5 Hz, 1H), 7.40 (d, J=8.0 Hz, 1H), 7.36 (t, J=6.0 Hz, 1H), 7.34-7.27 (m, 3H), 7.25-7.21 (m, 1H), 6.41 (s, 1H), 5.08 (s, 2H), 4.45 (d, J=13.3 Hz, 1H), 4.31 (d, J=10.7 Hz, 1H), 4.19-4.12 (m, 1H), 4.05 (t, J=6.6 Hz, 2H), 3.87 (s, 3H), 3.85-3.78 (m, 4H), 3.61 (br, 2H), 3.37 (t, J=10.6 Hz, 1H), 3.02-2.84 (m, 2H), 2.59-2.43 (m, 4H), 2.37 (s, 3H), 2.20-2.12 (m, 1H), 1.91-1.83 (m, 1H), 1.81-1.73 (m, 1H), 1.71-1.50 (m, 3H); ¹³C NMR (100 MHz, CDCl₃) δ 171.0, 169.2, 168.2, 153.5, 151.0, 148.3, 146.7, 140.8, 136.5, 134.3, 134.2, 130.3, 129.7, 129.1, 126.1, 125.1, 122.6, 122.5, 121.2, 119.7, 118.4, 122.6, 111.0, 105.6, 70.8, 57.7, 56.2, 53.2, 51.2, 46.1, 46.0, 45.7, 44.6, 42.4, 40.1, 23.2, 22.6, 19.1; MS (ES+): m/z=766 (M+H)⁺; LCMS (Method A): t_{R}=5.97 min.

### Example 143: tert-Butyl (S)-5-hydroxy-1-methyl-1,2-dihydro-3H-benzo[e]indole-3-carboxylate (141)

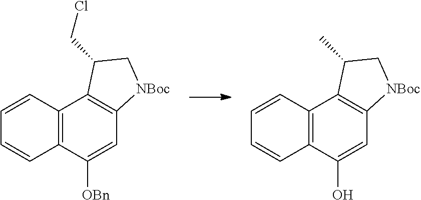 [SMILES: CC(C)(C)OC(=O)N1C[C@@H](CCl)c2c1cc(OCc1ccccc1)c1ccccc21 ; C[C@@H]1CN(C(=O)OC(C)(C)C)c2cc(O)c3ccccc3c21]

A solution of tert-Butyl (S)-5-(benzyloxy)-1-(chloromethyl)-1,2-dihydro-3H-benzo[e]indole-3-carboxylate (82) (100 mg, 0.236 mmol) in tetrahydrofuran (4 mL) was charged with ammonium formate (119 mg, 1.89 mmol) and palladium on activated charcoal (10 wt. % basis, 100 mg), followed by water (1 mL). The resulting mixture was heated to 65° C., under argon, for 3 h, after which TLC and LCMS showed completion of the reaction. After allowing the mixture to cool to ambient conditions, it was filtered through a pad of celite. The resulting filter cake was then washed with ethyl acetate (100 mL), water (100 mL) and methanol (10 mL). The organic phase was separated, and the aqueous phase was extracted with ethyl acetate (100 mL). The combined organic phases were then dried over magnesium sulfate, filtered and concentrated in vacuo to give a green solid residue, which was recrystallised from ethyl acetate/petroleum spirit, 40-60° C. to give the title compound (68 mg, 96%) as a white solid. This was then used in the subsequent step without any further purification.

¹H NMR (400 MHz, CDCl₃) δ 8.23-8.15 (m, 1H), 7.80 (br, 1H), 7.68 (d, J=8.4 Hz, 1H), 7.52-7.42 (m, 2H), 7.37-7.28 (m, 1H), 4.03-3.88 (m, 1H), 3.83-3.65 (m, 1H), 3.42 (t, J=11.0 Hz, 1H), 1.58 (s, 9H), 1.38 (d, J=6.8 Hz, 3H); MS (ES−): m/z=298 (M−1)⁻; LCMS (Method A): t_{R}=7.80 min.

### Example 144: (S)-1-Methyl-2,3-dihydro-1H-benzo[e]indol-5-ol hydrochloride (142)

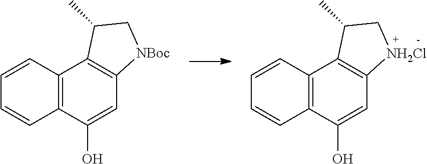

A solution of tert-butyl (S)-5-hydroxy-1-methyl-1,2-dihydro-3H-benzo[e]indole-3-carboxylate (141) (68 mg, 0.227 mmol) in 1,4-dioxane (1 mL) was charged with hydrochloric acid (4 M in 1,4-dioxane, 1 mL) and stirred at room temperature for 4 h, after which LCMS confirmed completion of reaction. The resulting mixture was concentrated in vacuo, then charged with diethyl ether and concentrated again, then subjected to strong vacuum for 1 h. The resulting navy blue residue (unstable) was used in the next step without further purification.

MS (ES+): m/z=200 (M+H)⁺; LCMS (Method A): t_{R}=4.70 min.

### Example 145: (S)-3-((3-(2-((S)-5-Hydroxy-1-methyl-1,2-dihydro-3H-benzo[e]indol-3-yl)-2-oxoethyl)benzyl)oxy)-2-methoxy-7,8,9,10-tetrahydrobenzo[e]pyrido[1,2-a][1,4]diazepine-6,12(5H,6aH)-dione (143)

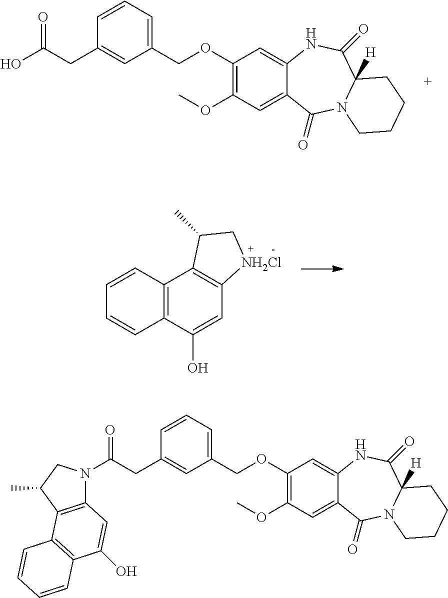

A solution of (S)-2-(3-(((2-methoxy-6,12-dioxo-5,6,6a,7,8,9,10,12-octahydrobenzo[e]pyrido[1,2-a][1,4]diazepin-3-yl)oxy)methyl)phenyl)acetic acid (138) (74 mg, 0.174 mmol) in N,N-dimethylacetamide (1 mL) was charged to (S)-1-methyl-2,3-dihydro-1H-benzo[e]indol-5-ol hydrochloride (142) (54 mg, 0.227 mmol), followed immediately by N-(3-dimethylaminopropyl)-N′-ethylcarbodiimide hydrochloride (100 mg, 0.523 mmol), and the resulting blue-green solution was stirred at room temperature for 16 h, whereupon it was diluted into ethyl acetate (100 mL) and washed with cold brine (2×50 mL), then dried over magnesium sulfate, filtered and concentrated in vacuo. Purification by flash column chromatography (silica), eluting with ethyl acetate/petroleum spirit, 40-60° C. (98%) gave the title compound (35 mg, 33%) as a white solid.

¹H NMR (400 MHz, CDCl₃) δ 9.05 (s, 1H), 8.65 (s, 1H), 8.46 (s, 1H), 8.27 (d, J=8.3 Hz, 1H), 7.64 (d, J=8.2 Hz, 1H), 7.48 (s, 1H), 7.45-7.39 (m, 1H), 7.35 (td, J=8.2, 1.2 Hz, 2H), 7.30 (s, 1H), 7.22 (t, J=70.6 Hz, 1H), 6.97 (d, J=7.4 Hz, 1H), 6.68 (s, 1H), 5.50 (d, J=13.1 Hz, 1H), 5.10 (d, J=13.1 Hz, 1H), 4.46 (dd, J=13.6, 3.7 Hz, 1H), 4.18 (dt, J=11.0, 9.5 Hz, 2H), 3.93-3.89 (m, 4H), 3.85-3.76 (m, 3H), 3.50 (d, J=16.9 Hz, 1H), 2.93 (td, J=13.4, 3.8 Hz, 1H), 2.27 (d, J=12.8 Hz, 1H), 1.89-1.51 (m, 4H), 1.40 (d, J=6.7 Hz, 3H); ¹³C NMR (100 MHz, CDCl₃) δ 171.3, 168.8, 168.0, 153.8, 149.9, 147.4, 139.6, 135.4, 133.7, 133.0, 129.9, 129.7, 129.3, 126.5, 123.4, 123.1, 122.7, 120.6, 119.9, 112.2, 108.8, 100.5, 71.6, 57.4, 56.2, 51.2, 41.9, 40.3, 33.7, 23.2, 22.9, 21.8, 19.3; MS (ES+): m/z=606 (M+H)⁺; LCMS (Method A): t_{R}=7.27 min.

### Example 146: Allyl (6aS)—R—((R-(2-((S)-5-hydroxy-1-methyl-1,2-dihydro-3H-benzo[e]indol-3-yl)-2-oxoethyl)benzyl)oxy)-2-methoxy-12-oxo-6-((tetra-hydro-2H-pyran-2-yl)oxy)-6,6a,7,8,9,10-hexahydrobenzo[e]pyrido[1,2-a][1,4]diazepine-5(12H)-carboxylate (144)

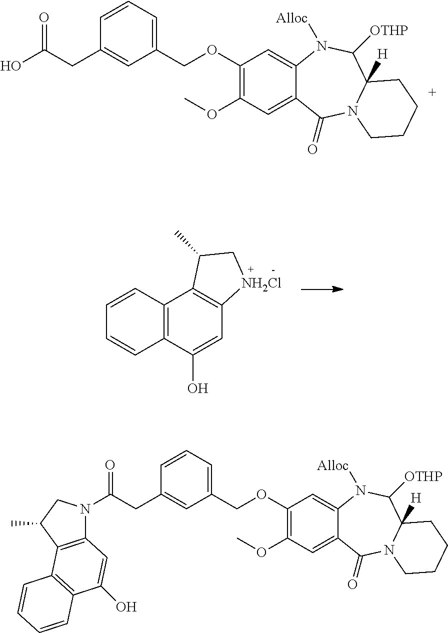

A solution of 2-(3-((((6aS)-5-((allyloxy)carbonyl)-2-methoxy-12-oxo-6-((tetrahydro-2H-pyran-2-yl)oxy)-5,6,6a,7,8,9,10,12-octahydrobenzo[e]pyrido[1,2-a][1,4]diazepin-3-yl)oxy)methyl)phenyl)acetic acid (86) (167 mg, 0.28 mmol) in N,N-dimethylacetamide (2 mL) was charged to (S)-1-methyl-2,3-dihydro-1H-benzo[e]indol-5-ol hydrochloride (142) (65 mg, 0.28 mmol), followed immediately by N-(3-dimethylaminopropyl)-N′-ethylcarbodiimide hydrochloride (215 mg, 1.12 mmol), and the resulting mixture was stirred at room temperature for 5 h. TLC and LCMS showed completion of reaction, whereupon the mixture was diluted into ethyl acetate (100 mL), and washed with cold brine (2×50 mL). The organic phase was then dried over magnesium sulfate, filtered and concentrated in vacuo. Purification was enacted by flash column chromatography (silica), eluting with ethyl acetate/petroleum spirit, 40-60° C. (80%) to give the title compound (116 mg, 53%) as a grey solid.

MS (ES+): m/z=776 (M+H)⁺; LCMS (Method A): t_{R}=8.18 min.

### Example 147: (S)-3-((3-(2-((S)-5-Hydroxy-1-methyl-1,2-dihydro-3H-benzo[e]indol-3-yl)-2-oxoethyl)benzyl)oxy)-2-methoxy-7,8,9,10-tetrahydrobenzo[e]pyrido[1,2-a][1,4]diazepin-12(6aH)-one (145)

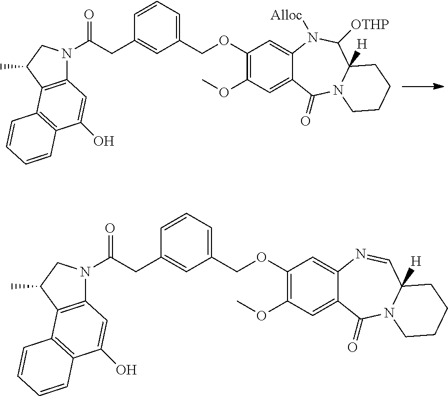

A solution of allyl (6aS)-3-((3-(2-((S)-5-hydroxy-1-methyl-1,2-dihydro-3H-benzo [e]indol-3-yl)-2-oxoethyl)benzyl)oxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (144) (116 mg, 0.15 mmol) in dichloromethane (1 mL) was charged with pyrrolidine (10 L) and tetrakis(triphenylphosphine)palladium(0) (3 mg) and the resulting mixture stirred for 25 min, after which it was diluted into dichloromethane (10 mL), filtered through a plug of celite, wash with dichloromethane and concentrated in vacuo. Diethyl ether (50 mL) was then charged and the mixture concentrated again. Purification by flash column chromatography (silica), eluting with methanol/ethyl acetate (3%) gave the title compound (54 mg, 61%) as a white solid.

¹H NMR (400 MHz, CDCl₃) δ 9.91 (br, 1H), 8.35 (s, 1H), 8.22 (d, J=8.3 Hz, 1H), 7.76 (d, J=5.7 Hz, 1H), 7.64 (d, J=8.3 Hz, 1H), 7.47-7.40 (m, 3H), 7.37-7.29 (m, 4H), 6.75 (s, 1H), 5.15 (q, J=12.7 Hz, 2H), 4.24-4.10 (m, 2H), 3.90 (d, J=3.0 Hz, 2H), 3.85 (s, 3H), 3.76 (dd, J=14.0, 3.7 Hz, 1H), 3.73-3.61 (m, 2H), 3.25-3.14 (m, 1H), 1.97-1.59 (m, 6H), 1.28 (d, J=6.8 Hz, 3H); ¹³C NMR (100 MHz, CDCl₃) δ 170.0, 167.5, 163.1, 154.0, 150.2, 148.2, 139.7, 136.9, 134.3, 129.8, 129.3, 128.7, 127.9, 126.7, 126.1, 123.7, 123.1, 123.0, 122.6, 121.4, 120.9, 111.7, 110.9, 100.8, 70.5, 57.7, 56.1, 49.5, 43.7, 39.7, 33.6, 24.4, 22.9, 21.4, 18.3; MS (ES+): m/z=590 (M+H)⁺; LCMS (Method A): t_{R}=7.13 min.

### Example 148: Methyl (S)-1-(5-methoxy-4-(4-methoxy-4-oxobutoxy)-2-nitrobenzoyl)piperidine-2-carboxylate (146)

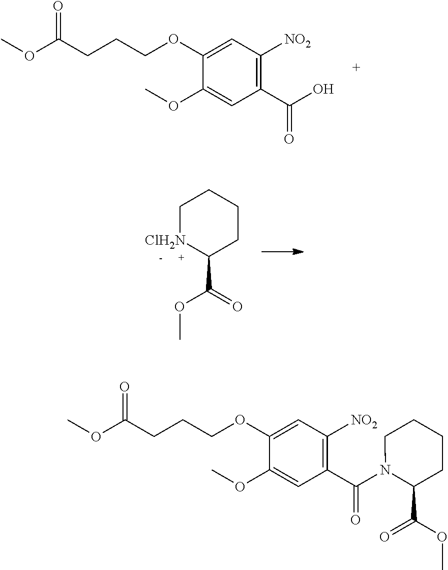

A slurry of 5-methoxy-4-(4-methoxy-4-oxobutoxy)-2-nitrobenzoic acid (3) (520 mg, 1.66 mmol) in dichloromethane (3.3 mL) was charged with triethylamine (972 μL, 6.97 mmol) and stirred. The resulting yellow solution was then charged with HATU (663 mg, 1.74 mmol) and stirred at room temperature for 5 min before adding methyl (2s)-piperidinecarboxylate hydrochloride (298 mg, 1.66 mmol) and stirring at room temperature for 4 h, after which the mixture was diluted into dichloromethane (100 mL) and washed with a saturated aqueous solution of sodium hydrogen carbonate (50 mL) followed by an aqueous solution of acetic acid (1% v/v, 100 mL), then dried over magnesium sulfate, filtered and concentrated in vacuo, to give the title compound (653 mg, 90%) as a yellow solid, which was used in the subsequent step without further purification.

MS (ES+): m/z=439 (M+H)⁺; LCMS (Method A): t_{R}=7.25 min.

### Example 14A: Methyl (S)-4-((2-methoxy-6,12-dioxo-5,6,6a,7,8,9,10,12-octahydrobenzo[e]pyrido[1,2-a][1,4]diazepin-3-yl)oxy)butanoate (147)

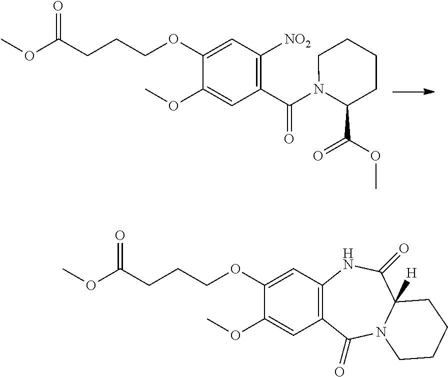

A solution of methyl (S)-1-(5-methoxy-4-(4-methoxy-4-oxobutoxy)-2-nitrobenzoyl)piperidine-2-carboxylate (146) (621 mg, 1.42 mmol) in tetrahydrofuran (3 mL) was charged with ammonium formate (715 mg, 11.3 mmol) and palladium on activated charcoal (10 wt. % basis, 62 mg), followed by water (1 mL) and the resulting mixture stirred under argon at 65° C. for 16 h. The mixture was then filtered through a pad of celite, and the filter cake washed with ethyl acetate (100 mL) and water (100 mL). The organic phase was then separated, washed twice with brine (50 mL), dried over magnesium sulfate, filtered and concentrated in vacuo. Purification by flash column chromatography (silica), eluting with ethyl acetate/petroleum spirit, 40-60° C. (from 50% to 100%), followed by methanol/ethyl acetate (from 0% to 65%) gave the title compound (52 mg, 10%) as an amber oil.

¹H NMR (400 MHz, CDCl₃) δ 8.77 (s, 1H), 7.30 (s, 1H), 6.49 (s, 1H), 4.45 (d, J=13.6 Hz, 1H), 4.10-4.06 (m, 1H), 3.99 (t, J=6.3 Hz, 2H), 3.82 (s, 3H), 3.71 (d, J=7.3 Hz, 1H), 3.63 (s, 3H), 2.98-2.86 (m, 1H), 2.48 (t, J=7.1 Hz, 2H), 2.23-2.14 (m, 1H), 1.95-1.82 (m, 1H), 1.82-1.46 (m, 5H); ¹³C NMR (100 MHz, CDCl₃) δ 173.4, 171.4, 168.3, 151.3, 146.6, 130.4, 119.5, 112.5, 104.7, 67.8, 56.1, 51.6, 51.3, 40.1, 38.6, 30.2, 24.1, 23.1, 22.7; MS (ES+): m/z=377 (M+H)⁺; LCMS (Method C): t_{R}=2.93 min.

### Example 150: (S)-4-((2-Methoxy-6,12-dioxo-5,6,6a,7,8,9,10,12-octahydrobenzo[e]pyrido[1,2-a][1,4]diazepin-3-yl)oxy)butanoic acid (148)

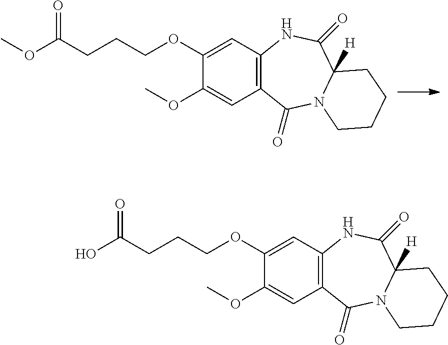 [SMILES: COC(=O)CCCOc1cc2c(cc1OC)C(=O)N1CCCC[C@H]1C(=O)N2 ; COc1cc2c(cc1OCCCC(=O)O)NC(=O)[C@@H]1CCCCN1C2=O]

A solution of methyl (S)-4-((2-methoxy-6,12-dioxo-5,6,6a,7,8,9,10,12-octahydrobenzo[e]pyrido[1,2-a][1,4]diazepin-3-yl)oxy)butanoate (147) (55 mg, 0.146 mmol) in tetrahydrofuran (1 mL) was charged with an aqueous solution of sodium hydroxide (0.5 M, 0.58 mL, 0.29 mmol) and stirred at room temperature for 2 h, upon which TLC and LCMS showed completion of reaction. The reaction mixture was then concentrated in vacuo, and taken up into water (50 mL) and ethyl acetate (50 mL), then acidified to pH=1 with an aqueous solution of hydrochloric acid (1 M). The phases were separated, and the aqueous extract was washed with ethyl acetate (100 mL). The combined organic extracts were then dried over magnesium sulfate, filtered, and concentrated in vacuo, the residue of which was then used in the subsequent step without further purification.

MS (ES+): m/z=363 (M+H)⁺; LCMS (Method C): t_{R}=2.55 min.

### Example 151: (S)-3-(4-((S)-1-(Chloromethyl)-5-hydroxy-1,2-dihydro-3H-benzo[e]indol-3-yl)-4-oxobutoxy)-2-methoxy-7,8,9,10-tetrahydrobenzo[e]-pyrido[1,2-a][1,4]diazepine-6,12(5H,6aH)-dione (14)

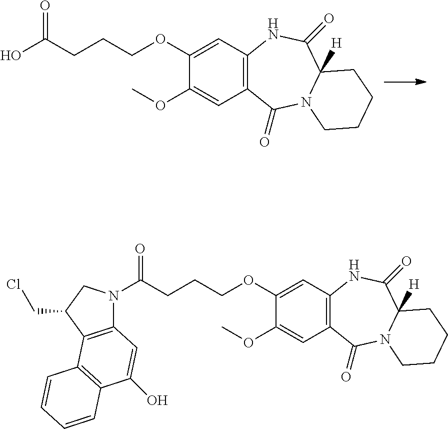 [SMILES: COc1cc2c(cc1OCCCC(=O)O)NC(=O)[C@@H]1CCCCN1C2=O ; COc1cc2c(cc1OCCCC(=O)N1C[C@@H](CCl)c3c1cc(O)c1ccccc31)NC(=O)[C@@H]1CCCCN1C2=O]

A solution of (S)-4-((2-methoxy-6,12-dioxo-5,6,6a,7,8,9,10,12-octahydrobenzo[e]pyrido[1,2-a][1,4]diazepin-3-yl)oxy)butanoic acid (148) (53 mg, 0.146 mmol) in N,N-dimethylacetamide (1 mL) was charged to (S)-1-(chloromethyl)-2,3-dihydro-H-benzo[e]indol-5-ol hydrochloride (11) (64 mg, 0.236 mmol), followed immediately by N-(3-dimethylaminopropyl)-N′-ethylcarbodiimide hydrochloride (84 mg, 0.439 mmol) and the resulting mixture stirred at room temperature for 16 h. The mixture was then taken up into ethyl acetate (100 mL) and washed with cold brine (2×50 mL), then dried over magnesium sulfate, filtered and concentrated in vacuo.

Purification by flash column chromatography (silica), eluting with ethyl acetate/petroleum spirit, 40-60° C. (from 15% to 100%) gave the title compound (5.6 mg, 7%) as a green solid.

¹H NMR (400 MHz, CDCl₃) δ 8.27 (br, 2H), 8.11 (s, 1H), 7.61 (d, J=8.6 Hz, 1H), 7.53-7.47 (m, 1H), 7.39-7.32 (m, 1H), 7.27 (s, 1H), 7.26 (br, 1H), 6.39 (s, 1H), 4.45 (d, J=14.1 Hz, 1H), 4.18-4.04 (m, 2H), 3.98 (br, 1H), 3.89 (d, J=13.2 Hz, 2H), 3.73 (s, 3H), 3.35 (t, J=11.5 Hz, 1H), 2.96-2.85 (m, 2H), 2.69 (br, 1H), 2.40-2.20 (m, 1H), 2.16-2.08 (m, 1H), 1.96-1.73 (m, 4H), 1.70-1.48 (m, 4H); MS (ES+): m/z=578 (M+H)⁺; LCMS (Method C): t_{R}=3.42 min.

### Example 152: (S)-1-(Chloromethyl)-3-(4-(((S)-2-methoxy-6,12-dioxo-5,6,6a,7,8,9,10,12-octahydrobenzo[e]pyrido[1,2-a][1,4]diazepin-3-yl)oxy)butanoyl)-2,3-dihydro-1H-benzo[e]indol-5-yl 4-methylpiperazine-1-carboxylate (150)

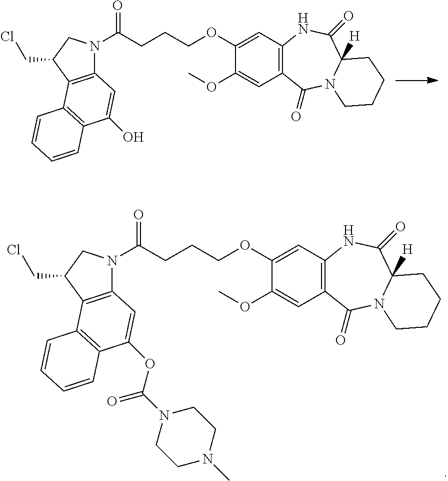 [SMILES: COc1cc2c(cc1OCCCC(=O)N1C[C@@H](CCl)c3c1cc(O)c1ccccc31)NC(=O)[C@@H]1CCCCN1C2=O ; COc1cc2c(cc1OCCCC(=O)N1C[C@@H](CCl)c3c1cc(OC(=O)N1CCN(C)CC1)c1ccccc31)NC(=O)[C@@H]1CCCCN1C2=O]

A solution of (S)-3-(4-((S)-1-(chloromethyl)-5-hydroxy-1,2-dihydro-3H-benzo[e]indol-3-yl)-4-oxobutoxy)-2-methoxy-7,8,9,10-tetrahydrobenzo [e]pyrido[1,2-a][1,4]diazepine-6,12(5H,6aH)-dione (149) (2.7 mg, 0.0047 mmol) in dichloromethane (100 L) was charged with 4-methyl-1-piperazinecarbonyl chloride hydrochloride (2.8 mg, 0.014 mmol), 4-(dimethylamino)pyridine (1.2 mg, 0.0098 mmol) and triethylamine (2.3 μL, 0.016 mmol) and stirred at room temperature for 1.5 h. The reaction mixture was subsequently diluted into dichloromethane (10 mL) washed with a saturated aqueous solution of sodium hydrogen carbonate (2×5 mL), dried over magnesium sulfate and concentrated in vacuo. The residue was then purified by flash column chromatography (silica), eluting with ethyl acetate (100%), followed by triethylamine/methanol/ethyl acetate (from 5:0:1 to 5:1:0), to give the title compound (1.6 mg, 48%) as a white solid.

¹H NMR (400 MHz, MeOD) δ 8.23 (s, 1H), 8.06 (d, J=6.3 Hz, 1H), 7.86 (dd, J=17.9, 8.4 Hz, 1H), 7.58 (t, J=7.5 Hz, 1H), 7.46 (t, J=8.0 Hz, 1H), 7.26 (s, 1H), 6.77 (d, J=6.6 Hz, 1H), 6.66 (s, 1H), 4.42-4.36 (m, 3H), 4.21-4.16 (m, 3H), 3.89 (br, 2H), 3.78 (s, 3H), 3.65-3.59 (m, 2H), 2.96-2.80 (m, 2H), 2.75-2.70 (m, 1H), 2.65-2.50 (m, 4H), 2.38 (s, 3H), 2.25 (t, J=6.4 Hz, 2H), 2.19-2.11 (m, 1H), 1.85-1.55 (m, 8H); MS (ES+): m/z=704 (M+H)⁺; LCMS (Method C): t_{R}=2.83 min.

### Example 153: Allyl (6aS)-3-(4-((S)-5-hydroxy-1-methyl-1,2-dihydro-3H-benzo[e]indol-3-yl)-4-oxobutoxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (151)

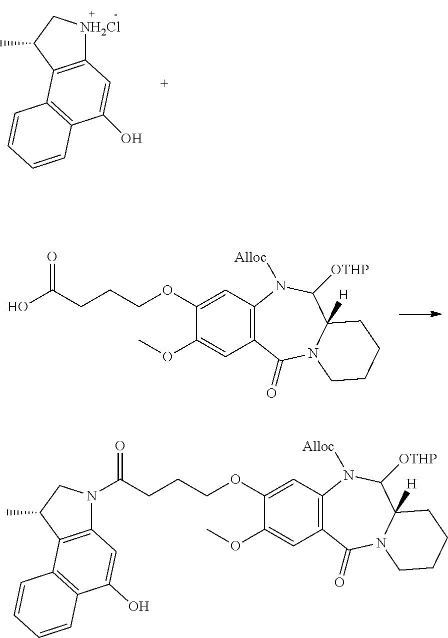

A solution of 4-(((6aS)-5-((allyloxy)carbonyl)-2-methoxy-12-oxo-6-((tetrahydro-2H-pyran-2-yl)oxy)-5,6,6a,7,8,9,10,12-octahydrobenzo[e]pyrido[1,2-a][1,4]diazepin-3-yl)oxy)butanoic acid (9) (100 mg, 0.188 mmol) in N,N-dimethylacetamide (0.5 mL) was charged to (S)-1-methyl-2,3-dihydro-1H-benzo[e]indol-5-ol hydrochloride (142) (54 mg, 0.227 mmol), followed immediately by N-(3-dimethylaminopropyl)-N′-ethylcarbodiimide hydrochloride (87 mg, 0.454 mmol) and the resulting mixture stirred at room temperature overnight. The reaction mixture was then diluted into ethyl acetate (100 mL) and washed with cold brine (2×50 mL), dried over magnesium sulfate, filtered and concentrated in vacuo. Purification by flash column chromatography (silica), eluting with ethyl acetate/petroleum spirit, 40-60° C. (from 10% to 100%) gave the title compound (36 mg, 22%) as a green solid.

¹H NMR (400 MHz, CDCl₃) δ 9.53 (br, 1H), 8.28-8.23 (m, 2H), 7.66 (d, J=8.4 Hz, 1H), 7.45 (t, J=70.6 Hz, 1H), 7.33 (t, J=70.6 Hz, 1H), 7.18 (s, 1H), 7.03 (s, 1H), 6.15 (d, J=11.0 Hz, 1H), 5.97 (d, J=9.7 Hz, 1H), 5.70 (br, 1H), 5.26-4.93 (m, 3H), 4.60-4.45 (m, 1H), 4.24 (d, J=13.1 Hz, 3H), 4.15 (d, J=10.3 Hz, 1H), 3.87 (s, 3H), 3.80 (d, J=10.5 Hz, 3H), 3.65 (br, 1H), 3.47 (dd, J=10.1, 6.1 Hz, 1H), 3.08 (d, J=13.2 Hz, 1H), 2.79-2.57 (m, 2H), 2.30 (dd, J=13.3, 7.0 Hz, 2H), 1.82-1.36 (m, 12H), 1.35 (d, J=6.6 Hz, 3H); ¹³C NMR (100 MHz, CDCl₃) δ 171.3, 169.3, 156.2, 153.7, 149.2, 143.1, 139.7, 132.0, 129.9, 126.8, 123.0, 122.6, 120.7, 117.2, 114.3, 110.6, 110.3, 100.5, 94.6, 83.9, 68.1, 62.8, 60.4, 57.4, 56.1, 55.7, 38.8, 33.6, 30.9, 30.4, 25.2, 23.3, 21.7, 21.0, 18.2; MS (ES+): m/z=714 (M+H)⁺; LCMS (Method C): t_{R}=3.92 min.

### Example 154: (S)-3-(4-((S)—S-Hydroxy-1-methyl-1,2-dihydro-3H-benzo[e]indol-3-yl)-4-oxobutoxy)-2-methoxy-7,8,9,10-tetrahydrobenzo[e]pyrido[1,2-a][1,4]diazepin-12(6aH)-one (152)

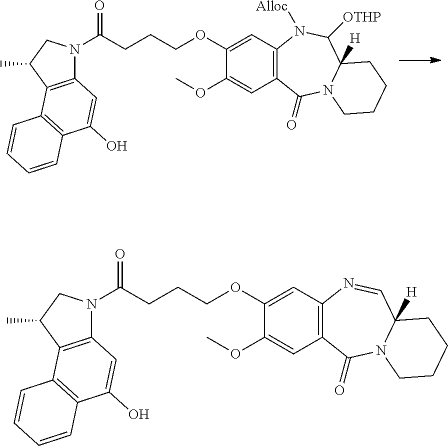

A solution of allyl (6aS)-3-(4-((S)-5-hydroxy-1-methyl-1,2-dihydro-3H-benzo[e]indol-3-yl)-4-oxobutoxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (151) (36 mg, 0.050 mmol) in dichloromethane (1 mL) was charged with pyrrolidine (5 μL, 0.061 mmol), and tetrakis(triphenylphosphine)palladium(0) (5.8 mg, 0.005 mmol) and stirred at room temperature for 40 min, whereupon it was concentrated in vacuo.

Purification by flash column chromatography (silica), eluting with acetone/dichloromethane (from 0% to 100%) gave the title compound (16 mg, 59%) as a cream solid.

¹H NMR (400 MHz, acetone-d₆) δ 9.20 (s, 1H), 8.19 (d, J=8.4 Hz, 1H), 8.13 (s, 1H), 7.97 (d, J=5.7 Hz, 1H), 7.75 (d, J=8.3 Hz, 1H), 7.46 (t, J=7.5 Hz, 1H), 7.34 (s, 1H), 7.33-7.28 (m, 1H), 6.82 (s, 1H), 4.36 (t, J=90.5 Hz, 1H), 4.26-4.05 (m, 3H), 3.93 (d, J=10.2 Hz, 1H), 3.86 (s, 3H), 3.82 (br, 1H), 3.79-3.71 (m, 2H), 3.21-3.11 (m, 1H), 2.73-2.62 (m, 1H), 2.22 (t, J=6.6 Hz, 2H), 2.00-1.55 (m, 6H), 1.34 (d, J=6.8 Hz, 3H); MS (ES+): m/z=528 (M+H)⁺; LCMS (Method A): t_{R}=7.02 min.

### Example 155: ((Allyloxy)carbonyl)-L-valine (153)

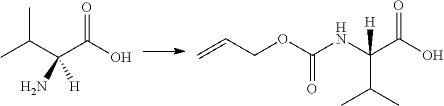

A solution of L-valine (33.0 g, 282 mmol) and potassium carbonate (58.4 g, 423 mmol) in tetrahydrofuran (500 mL) and water (500 mL) was charged with allyl chloroformate (40.75 g, 338 mmol) dropwise, and stirred at room temperature for 18h. The resulting mixture was partially concentrated in vacuo, then extracted with diethyl ether (300 mL). The aqueous phase was acidified to pH=2 with concentrated hydrochloric acid, then extracted with dichloromethane (3×300 mL). The combined organic phases were then washed with brine, dried over magnesium sulfate, filtered and then concentrated in vacuo to give the title compound (53.0 g, 94%) as a colourless oil.

¹H NMR (400 MHz, DMSO-d₆) δ 12.55 (s, 1H), 7.40 (d, J=8.6 Hz, 1H), 5.91 (ddt, J=16.2, 10.6, 5.2 Hz, 1H), 5.30 (dd, J=17.2, 1.8 Hz, 1H), 5.18 (dd, J=10.6, 1.8 Hz, 1H), 4.52-4.44 (m, 2H), 3.85 (dd, J=8.6, 6.0 Hz, 1H), 2.09-1.99 (m, 1H), 0.90-0.86 (m, 6H); MS (ES+): m/z=202 (M+H)⁺; LCMS (Method F): t_{R}=3.10 min.

### Example 156: 2,5-Dioxopyrrolidin-1-yl ((allyloxy)carbonyl)-L-valinate (154)

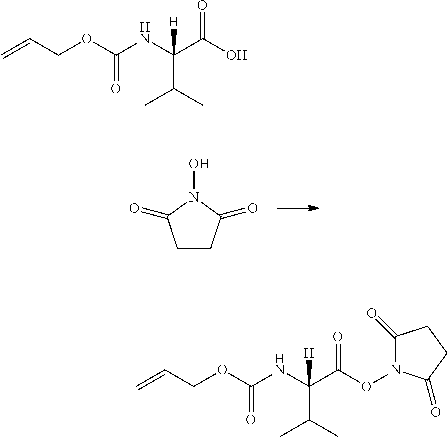 [SMILES: C=CCOC(=O)N[C@H](C(=O)O)C(C)C ; O=C1CCC(=O)N1O ; C=CCOC(=O)N[C@H](C(=O)ON1C(=O)CCC1=O)C(C)C]

A solution of ((allyloxy)carbonyl)-L-valine (153) (53.0 g, 263 mmol) and N-hydroxysuccinimide (30.3 g, 263 mmol) in anhydrous tetrahydrofuran (1 L) was charged with N,N-dicyclohexylcarbodiimide (54.4 g, 263 mmol) and the resulting mixture stirred at room temperature for 18 h. The mixture was then filtered, and the residue washed with tetrahydrofuran (300 mL). The combined filtrate was then concentrated in vacuo. Dichloromethane was then added to the residue and the resulting slurry was left to stand at 0° C. The suspension was filtered and washed with cold dichloromethane. The filtrate was then concentrated in vacuo to afford the title compound (60.0 g, 76%) as a viscous, colourless oil.

¹H NMR (400 MHz, DMSO-d₆) δ 7.99 (d, J=8.2 Hz, 1H), 5.93 (ddt, J=16.2, 10.6, 5.2 Hz, 1H), 5.38-5.26 (m, 1H), 5.19 (d, J=10.6 Hz, 1H), 4.53 (d, J=5.4 Hz, 2H), 4.33 (dd, J=8.2, 6.2 Hz, 1H), 2.81 (s, 4H), 2.19 (q, J=6.8 Hz, 1H), 1.01 (d, J=6.8 Hz, 6H); MS (ES+): m/z=299 (M+H)⁺; LCMS (Method F): t_{R}=3.57 min.

### Example 157: ((Allyloxy)carbonyl)-L-valyl-L-alanine (155)

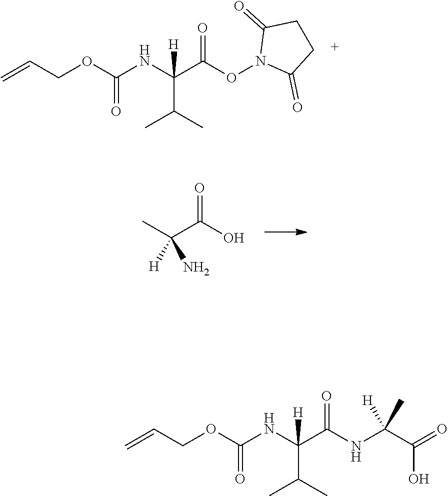

A solution of L-alanine (18.8 g, 211 mmol) and sodium hydrogen carbonate (18.6 g, 221 mmol) in tetrahydrofuran (100 mL) and water (200 mL) was charged with a solution of 2,5-dioxopyrrolidin-1-yl ((allyloxy)carbonyl)-L-valinate (154) (60.0 g, 201 mmol) in tetrahydrofuran (100 mL). The resulting mixture was stirred for 72 h and then partially concentrated in vacuo. A saturated aqueous solution of citric acid was used to acidify to pH=3-4, and the mixture was then extracted with ethyl acetate (6×150 mL). The combined organic extracts were washed with water (200 mL), brine (200 mL) and dried over magnesium sulfate, then filtered and concentrated in vacuo to afford a white solid, which was triturated with diethyl ether, giving the title compound (38.0 g, 69%) as a white powder.

¹H NMR (400 MHz, DMSO-d₆) δ 12.47 (s, 1H), 8.17 (d, J=7.0 Hz, 1H), 7.15 (d, J=9.0 Hz, 1H), 5.93-5.85 (m, 1H), 5.29 (d, J=17.2 Hz, 1H), 5.17 (d, J=10.4 Hz, 1H), 4.46 (d, J=50.2 Hz, 2H), 4.22-4.15 (m, 1H), 3.87 (t, J=8.0 Hz, 1H), 1.95 (dd, J=14.6, 7.6 Hz, 1H), 1.26 (d, J=70.2 Hz, 3H), 0.88 (d, J=6.8 Hz, 3H), 0.83 (d, J=6.8 Hz, 3H); MS (ES+): m/z=273 (M+H)⁺; LCMS (Method F): t_{R}=2.67 min.

### Example 158: Allyl ((S)-1-(((S)-1-((4-(hydroxymethyl)phenyl)amino)-1-oxopropan-2-yl)amino)-3-methyl-1-oxobutan-2-yl)carbamate (156)

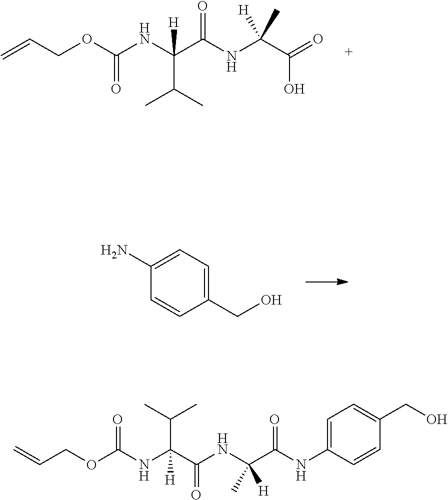 [SMILES: C=CCOC(=O)N[C@H](C(=O)N[C@@H](C)C(=O)O)C(C)C ; Nc1ccc(CO)cc1 ; C=CCOC(=O)N[C@H](C(=O)N[C@@H](C)C(=O)Nc1ccc(CO)cc1)C(C)C]

A solution of ((allyloxy)carbonyl)-L-valyl-L-alanine (155) (38.0 g, 140 mmol) in anhydrous tetrahydrofuran (800 mL) was charged with (4-aminophenyl)methanol (18.1 g, 147 mmol) and 2-ethoxy-1-ethoxycarbonyl-1,2-dihydroquinoline (36.2 g, 147 mmol). The resulting mixture was stirred at room temperature for 72 h. The solvent was then evaporated in vacuo to give a pale brown solid. This residue was then triturated with diethyl ether and filtered, washed with an excess of diethyl ether to afford the title compound (40.0 g, 76%) as a white solid.

¹H NMR (400 MHz, DMSO-d₆) δ 9.89 (s, 1H), 8.13 (d, J=70.2 Hz, 1H), 7.53 (d, J=8.0 Hz, 2H), 7.24 (dd, J=8.8, 2.6 Hz, 3H), 5.91 (td, J=10.8, 5.2 Hz, 1H), 5.30 (d, J=17.0 Hz, 1H), 5.17 (d, J=10.4 Hz, 1H), 5.08 (d, J=5.6 Hz, 1H), 4.52-4.45 (m, 2H), 4.43 (d, J=30.6 Hz, 3H), 3.94-3.82 (m, 1H), 1.98 (d, J=6.8 Hz, 1H), 1.30 (d, J=7.0 Hz, 3H), 0.89 (d, J=6.8 Hz, 3H), 0.84 (d, J=6.8 Hz, 3H); MS (ES+): m/z=378 (M+H)⁺; LCMS (Method F): t_{R}=2.98 min.

### Example 159: Allyl ((S)-1-methyl-1-(((S)-1-((4-((((4-nitrophenoxy)-carbonyl)oxy)methyl)phenyl)amino)-1-oxopropan-2-yl)amino)-1-oxobutan-2-yl)carbamate (157)

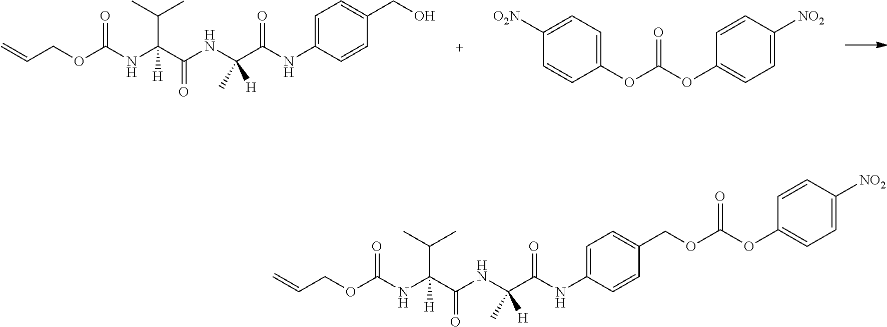 [SMILES: C=CCOC(=O)N[C@H](C(=O)N[C@@H](C)C(=O)Nc1ccc(CO)cc1)C(C)C ; O=C(Oc1ccc([N+](=O)[O-])cc1)Oc1ccc([N+](=O)[O-])cc1 ; C=CCOC(=O)N[C@H](C(=O)N[C@@H](C)C(=O)Nc1ccc(COC(=O)Oc2ccc([N+](=O)[O-])cc2)cc1)C(C)C]

A solution of allyl ((S)-1-(((S)-1-((4-(hydroxymethyl)phenyl)amino)-1-oxopropan-2-yl)amino)-3-methyl-1-oxobutan-2-yl)carbamate (156) (40.0 g, 106 mmol) in anhydrous tetrahydrofuran (1 L) was charged with bis(4-nitrophenyl) carbonate (64.5 g, 212 mmol) and triethylamine (21.5 g, 212 mmol). The resulting mixture was stirred at room temperature for 1.5 h and then concentrated in vacuo. The mixture was triturated with ethyl acetate (2×100 mL) and methanol/dichloromethane (10%, 3×50 mL) to afford the title compound (20.0 g, 35%) as a white solid.

¹H NMR (400 MHz, DMSO-d₆) δ 10.07 (s, 1H), 8.31 (d, J=8.6 Hz, 2H), 8.17 (d, J=6.8 Hz, 1H), 7.64 (d, J=8.2 Hz, 2H), 7.56 (d, J=8.6 Hz, 2H), 7.41 (d, J=8.0 Hz, 2H), 7.24 (d, J=8.8 Hz, 1H), 5.94-5.86 (m, 1H), 5.30 (d, J=17.0 Hz, 1H), 5.24 (s, 2H), 5.17 (d, J=10.4 Hz, 1H), 4.48 (d, J=50.2 Hz, 2H), 4.45-4.32 (m, 1H), 3.90 (t, J=70.8 Hz, 1H), 1.98 (dd, J=8.6, 5.2 Hz, 1H), 1.32 (d, J=7.0 Hz, 3H), 0.89 (d, J=6.8 Hz, 3H), 0.84 (d, J=6.8 Hz, 3H); MS (ES+): m/z=543 (M+H)⁺; LCMS (Method F): t_{R}=3.83 min.

### Example 160: 4-((S)-2-((S)-2-(((Allyloxy)carbonyl)amino)-3-methylbutan-amido)propanamido)benzyl (2-((S)-2-(hydroxymethyl)piperidine-1-carbonyl)-4-methoxy-5-((triisopropylsilyl)oxy)phenyl)carbamate (158)

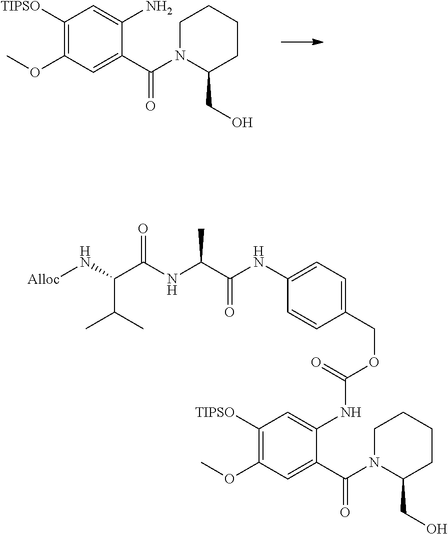

A solution of (S)-(2-amino-5-methoxy-4-((triisopropylsilyl)oxy)phenyl)(2-(hydroxymethyl)piperidin-1-yl)methanone (69) (5.0 g, 11.5 mmol) in N,N-dimethylformamide (23 mL) was charged with allyl ((S)-3-methyl-1-(((S)-1-((4-((((4-nitrophenoxy)carbonyl)oxy)methyl)phenyl)amino)-1-oxopropan-2-yl)amino)-1-oxobutan-2-yl)carbamate (157) (6.2 g, 11.5 mmol) and 1-hydroxybenzotriazole hydrate (1.5 g, 11.5 mmol), and the resulting mixture was heated to 60° C. under argon, for 16 h. The mixture was allowed to cool to room temperature, then diluted into ethyl acetate (500 mL) and washed with cold brine (3×100 mL). The organic phase was dried over magnesium sulfate, filtered and concentrated in vacuo. Purification by flash column chromatography (silica), eluting with ethyl acetate/petroleum spirit, 40-60° C. (from 10% to 90%) gave the title compound (7.49 g, 78%) as a beige solid.

¹H NMR (400 MHz, CDCl₃) δ 9.12 (br, 1H), 8.13 (br, 1H), 7.96 (s, 2H), 7.47 (d, J=8.3 Hz, 2H), 7.22 (d, J=8.0 Hz, 2H), 6.73 (s, 1H), 5.80 (d, J=8.2 Hz, 1H), 5.24 (d, J=17.3 Hz, 1H), 5.14 (d, J=10.2 Hz, 1H), 5.04 (q, J=12.4 Hz, 2H), 4.67-4.58 (m, 1H), 4.55-4.44 (m, 2H), 4.09 (dd, J=12.3, 6.6 Hz, 1H), 3.80 (t, J=10.6 Hz, 1H), 3.71 (s, 3H), 3.53 (br, 1H), 2.14-1.98 (m, 1H), 1.65-1.50 (m, 4H), 1.34 (d, J=6.9 Hz, 3H), 1.28-1.15 (m, 4H), 1.05 (d, J=7.4 Hz, 22H), 0.87 (dd, J=11.4, 6.8 Hz, 6H); ¹³C NMR (100 MHz, CDCl₃) δ 171.9, 170.6, 170.5, 162.6, 156.5, 153.9, 137.9, 137.1, 132.5, 132.1, 128.7, 127.5, 120.1, 119.8, 117.7, 111.0, 66.2, 65.9, 60.3, 56.3, 55.9, 49.5, 36.5, 31.4, 31.2, 25.6, 19.8, 19.6, 19.2, 17.9, 17.8, 12.8; MS (ES+): m/z=840 (M+H)⁺; LCMS (Method A): t_{R}=9.53 min.

### Example 161: 4-((S)-2-((S)-2-(((Allyloxy)carbonyl)amino)-3-methylbutanamido)propanamido)benzyl (5-hydroxy-2-((S)-2-(hydroxymethyl)piperidine-1-carbonyl)-4-methoxyphenyl)carbamate (159)

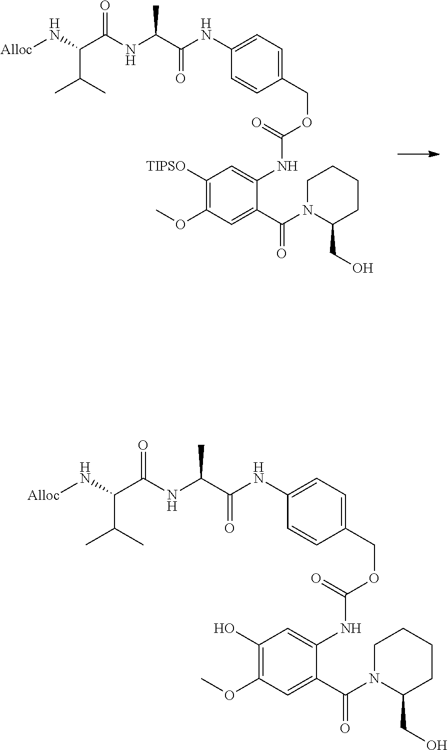

A solution of 4-((S)-2-((S)-2-(((allyloxy)carbonyl)amino)-3-methylbutanamido)-propanamido)benzyl (2-((S)-2-(hydroxymethyl)piperidine-1-carbonyl)-4-methoxy-5-((triisopropylsilyl)oxy)phenyl)carbamate (158) (1.89 g, 2.25 mmol) in tetrahydrofuran (6 mL) was charged with tetrabutylammonium fluoride (1 M in tetrahydrofuran, 3.38 mL, 3.38 mmol) and stirred at room temperature for 10 min. The reaction mixture was subsequently concentrated in vacuo to give an orange solid, which was purified by flash column chromatography (silica), eluting with methanol/ethyl acetate (from 0% to 10%) to give the title compound (1.41 g, 92%) as a cream solid.

¹H NMR (400 MHz, CDCl₃) δ 9.18 (s, 1H), 8.25 (s, 1H), 7.66 (d, J=6.9 Hz, 1H), 7.46 (s, 1H), 7.36 (d, J=8.0 Hz, 2H), 7.14 (d, J=8.2 Hz, 2H), 6.73 (s, 1H), 5.85 (ddd, J=16.2, 11.2, 6.6 Hz, 2H), 5.25 (d, J=17.3 Hz, 1H), 5.15 (d, J=10.6 Hz, 1H), 5.08-4.94 (m, 2H), 4.73-4.61 (m, 1H), 4.59-4.43 (m, 2H), 4.11 (t, J=70.2 Hz, 1H), 3.97 (br, 1H), 3.88-3.76 (m, 1H), 3.74 (s, 3H), 3.51 (ddd, J=17.5, 10.7, 9.8 Hz, 1H), 2.93 (br, 1H), 2.48 (br, 1H), 2.03 (dd, J=12.7, 7.3 Hz, 2H), 1.59 (br, 4H), 1.35 (d, J=6.8 Hz, 5H), 0.87 (apparent t, J=6.7 Hz, 6H); ¹³C NMR (100 MHz, CDCl₃) δ 172.1, 170.9, 170.5, 156.6, 154.3, 147.6, 143.5, 137.7, 132.5, 132.2, 129.5, 128.6, 120.1, 117.8, 110.2, 66.3, 65.9, 60.3, 56.2, 50.6, 49.6, 31.3, 25.6, 19.5, 19.1, 17.9, 17.8; MS (ES+): m/z=684 (M+H)⁺; LCMS (Method A): t_{R}=6.13 min.

### Example 162: Methyl 2-(3-((5-((((4-((S)-2-((S)-2-(((allyloxy)carbonyl)-amino)-3-methylbutanamido)propanamido)benzyl)oxy)carbonyl)amino)-4-((S)-2-(hydroxymethyl)piperidine-1-carbonyl)-2-methoxyphenoxy)methyl)phenyl)acetate (160)

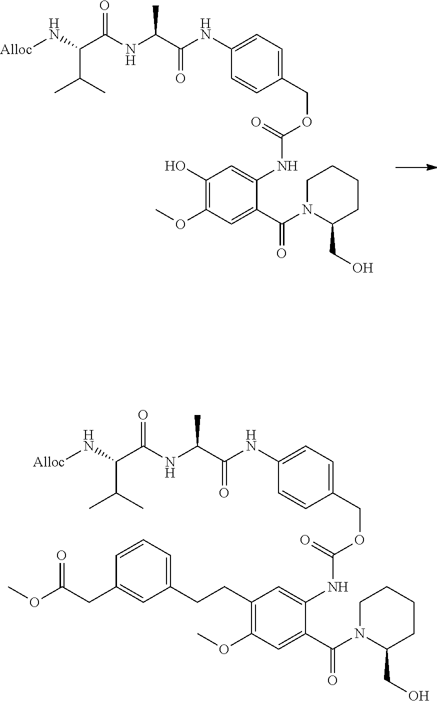

A solution of 4-((S)-2-((S)-2-(((allyloxy)carbonyl)amino)-3-methylbutanamido)propanamido)benzyl (5-hydroxy-2-((S)-2-(hydroxymethyl)piperidine-1-carbonyl)-4-methoxyphenyl)carbamate (159) (2.18 g, 3.19 mmol) in N,N-dimethylformamide (7 mL) was charged with potassium carbonate (661 mg, 4.78 mmol) and 3-(bromomethyl)-benzeneacetic acid methyl ester (813 mg, 3.35 mmol) and the resulting mixture stirred at room temperature for 16 h. The mixture was then diluted into ethyl acetate (500 mL) and washed with cold brine (2×100 mL), then dried over magnesium sulfate, filtered and concentrated in vacuo. Purification by flash column chromatography (silica), eluting with methanol/ethyl acetate (from 0% to 10%) gave the title compound (2.03 g, 75%) as a pale yellow solid.

¹H NMR (400 MHz, CDCl₃) δ 9.01 (s, 1H), 8.27 (s, 1H), 7.67 (s, 1H), 7.48 (d, J=8.2 Hz, 2H), 7.44-7.18 (m, 7H), 6.80 (s, 1H), 5.93-5.80 (m, 1H), 5.74 (d, J=8.1 Hz, 1H), 5.26 (d, J=14.5 Hz, 1H), 5.17 (d, J=10.2 Hz, 1H), 5.06 (q, J=12.4 Hz, 3H), 4.69-4.60 (m, 1H), 4.60-4.46 (m, 2H), 3.83 (br, 1H), 3.79 (s, 3H), 3.66 (s, 3H), 3.61 (s, 2H), 3.51 (br, 1H), 2.93 (br, 1H), 2.09-2.04 (m, 1H), 1.66-1.54 (m, 4H), 1.40 (br, 1H), 1.36 (d, J=6.9 Hz, 3H), 0.89 (dd, J=10.0, 6.8 Hz, 6H); ¹³C NMR (100 MHz, CDCl₃) δ 171.9, 171.2, 170.5, 156-5, 154.1, 149.5, 145.3, 137.9, 136.7, 134.2, 132.5, 132.1, 129.0, 128.8, 128.7, 128.6, 126.6, 119.9, 117.9, 70.6, 66.4, 66.0, 60.4, 57.7, 56.4, 52.1, 49.6, 41.1, 31.2, 25.7, 21.0, 19.6, 19.2, 17.9, 17.8; MS (ES+): m/z=846 (M+H)⁺; LCMS (Method A): t_{R}=7.10 min.

### Example 163: 2-(3-((5-((((4-((S)-2-((S)-2-(((Allyloxy)carbonyl)amino)-3-methylbutanamido)propanamido)benzyl)oxy)carbonyl)amino)-4-((S)-2-(hydroxymethyl)piperidine-1-carbonyl)-2-methoxyphenoxy)methyl)-phenyl)acetic acid (161)

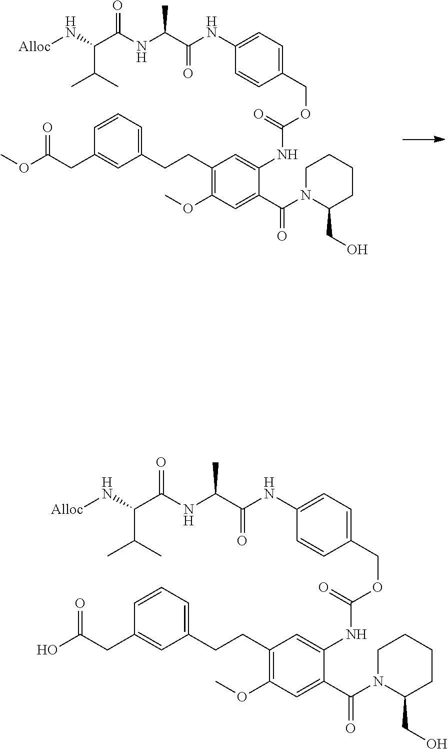

A solution of methyl 2-(3-((5-((((4-((S)-2-((S)-2-(((allyloxy)carbonyl)amino)-3-methylbutanamido)propanamido)benzyl)oxy)carbonyl)amino)-4-((S)-2-(hydroxymethyl)piperidine-1-carbonyl)-2-methoxyphenoxy)methyl)phenyl)acetate (160) (2.03 g, 2.40 mmol) in tetrahydrofuran (34 mL) was charged with an aqueous solution of sodium hydroxide (0.5 M, 9.6 mL, 4.80 mmol) dropwise, and stirred at room temperature. The reaction progress was closely monitored by LCMS and after 2 h, quenched by cautious addition of a saturated aqueous solution of citric acid (adjusted to pH=3-4). The resulting mixture was partially concentrated in vacuo, then extracted with ethyl acetate (2×250 mL). The combined organic phases were washed with brine (100 mL), then dried over magnesium sulfate, filtered and concentrated in vacuo. Purification by flash column chromatography (silica), eluting with methanol/ethyl acetate (from 2% to 10%) gave the title compound (1.19 g, 59%) as a pale yellow solid.

¹H NMR (400 MHz, CDCl₃) δ 8.96 (s, 1H), 8.07 (s, 1H), 7.43 (d, J=70.8 Hz, 2H), 7.34 (d, J=7.5 Hz, 1H), 7.29 (d, J=7.4 Hz, 1H), 7.27-7.20 (m, 4H), 7.17 (d, J=70.8 Hz, 1H), 6.74 (s, 1H), 5.92-5.81 (m, 1H), 5.67-5.57 (m, 1H), 5.27 (d, J=17.1 Hz, 1H), 5.18 (d, J=10.8 Hz, 1H), 5.11-4.94 (m, 4H), 4.71-4.57 (m, 1H), 4.53 (t, J=50.1 Hz, 2H), 4.05 (t, J=6.6 Hz, 1H), 3.89-3.81 (m, 1H), 3.76 (s, 3H), 3.65-3.53 (m, 3H), 2.94 (br, 1H), 2.75-2.15 (br, 6H), 2.03 (dd, J=16.9, 11.5 Hz, 2H), 1.59-1.42 (br, 3H), 1.35 (d, J=7.0 Hz, 3H), 0.88 (apparent t, J=7.4 Hz, 6H); MS (ES+): m/z=832 (M+H)⁺; LCMS (Method A): t_{R}=6.75 min.

### Example 164: 2-(3-((((6aS)-5-(((4-((S)-2-((S)-2-(((Allyloxy)carbonyl)-amino)-3-methylbutanamido)propanamido)benzyl)oxy)carbonyl)-6-hydroxy-2-methoxy-12-oxo-5,6,6a,7,8,9,10,12-octahydrobenzo[e]-pyrido[1,2-a][1,4]diazepin-3-yl)oxy)methyl)phenyl)acetic acid (162)

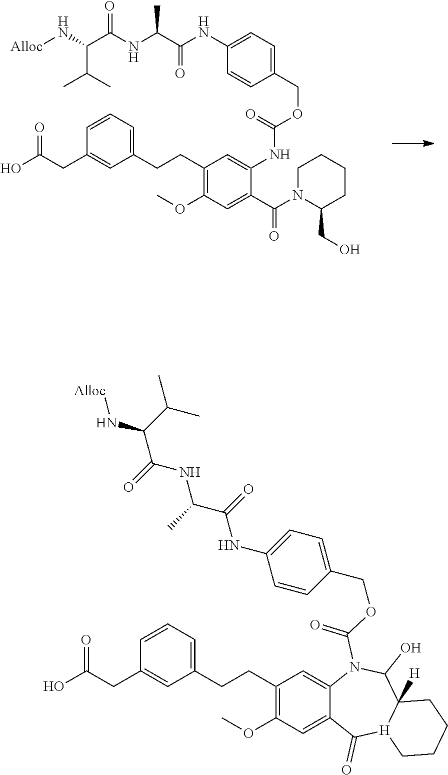

A solution of 2-(3-((5-((((4-((S)-2-((S)-2-(((allyloxy)carbonyl)amino)-3-methylbutanamido)propanamido)benzyl)oxy)carbonyl)amino)-4-(S)-2-(hydroxymethyl)piperidine-1-carbonyl)-2-methoxyphenoxy)methyl)phenyl)acetic acid (161) (670 mg, 0.805 mmol) in anhydrous dichloromethane (5 mL) was cooled to −5° C. and charged with Dess-Martin periodinane (678 mg, 1.60 mmol). After 15 min, the reaction mixture was allowed to warm to room temperature and stirred for a further 1 h, whilst monitoring the reaction progress by LCMS. The reaction was quenched by addition of a saturated aqueous solution of sodium metabisulfite, then extracted with dichloromethane (2×100 mL). The combined organic phases were dried over magnesium sulfate and concentrated in vacuo. The resulting residue was then purified by flash column chromatography (silica), eluting with methanol/ethyl acetate (from 0% to 10%) to give the title compound (431 mg, 65%) as an off-white solid. MS (ES−): m/z=828 (M−1)⁻, MS (ES+): m/z=830 (M+H)⁺; LCMS (Method A): t_{R}=6.63 min.

### Example 165: 4-((S)-2-((S)-2-(((Allyloxy)carbonyl)amino)-3-methylbutanamido)propanamido)benzyl (6aS)-3-((3-(2-((S)-1-(chloromethyl)-5-hydroxy-1,2-dihydro-3H-benzo[e]indol-3-yl)-2-oxoethyl)benzyl)oxy)-6-hydroxy-2-methoxy-12-oxo-6,6a,7,8,9,10-hexahydrobenzo[e]pyrido[1,2-a][1,4]diazepine-5(12H)-carboxylate (163)

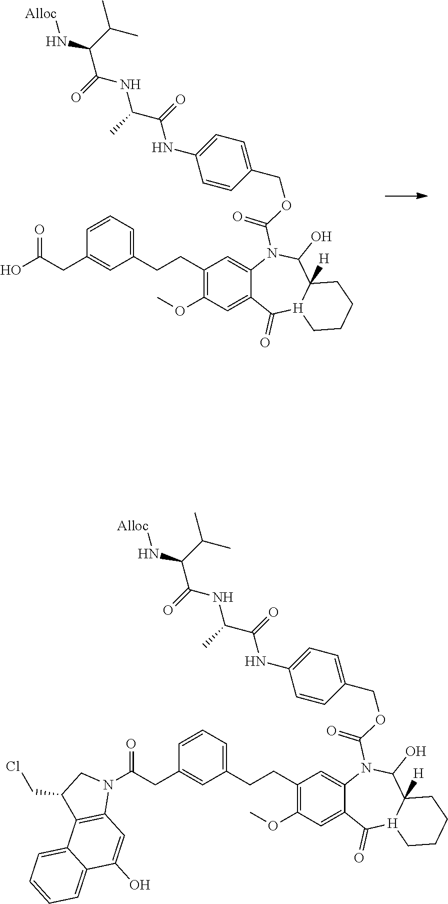

A solution of 2-(3-((((6aS)-5-(((4-((S)-2-((S)-2-(((allyloxy)carbonyl)amino)-3-methylbutanamido)propanamido)benzyl)oxy)carbonyl)-6-hydroxy-2-methoxy-12-oxo-5,6,6a,7,8,9,10,12-octahydrobenzo[e]pyrido[1,2-a][1,4]diazepin-3-yl)oxy)methyl)phenyl)acetic acid (162) (253 mg, 0.305 mmol) in N,N-dimethylacetamide (1 mL) was charged to (S)-1-(chloromethyl)-2,3-dihydro-1H-benzo[e]indol-5-ol hydrochloride (11) (115 mg, 0.427 mmol), followed immediately by N-(3-dimethylaminopropyl)-N′-ethylcarbodiimide hydrochloride (263 mg, 1.37 mmol) and the resulting mixture was sonicated, then stirred at room temperature for 3 h. The mixture was then diluted into ethyl acetate (100 mL) and washed with cold brine (2×20 mL), then dried over magnesium sulfate, filtered and concentrated in vacuo. Purification by flash column chromatography (silica), eluting with ethyl acetate/petroleum spirit, 40-60° C. (from 50% to 100%) gave the title compound (195 mg, 61%) as a pale green solid.

MS (ES+): m/z=1045 (M+H)⁺; LCMS (Method A): t_{R}=7.78 min.

### Example 166: 4-((S)-2-((S)-2-(((Allyloxy)carbonyl)amino)-3-methylbutan-amido)propanamido)benzyl (6aS)-3-((3-(2-((S)-1-(chloromethyl)-5-((4-methylpiperazine-1-carbonyl)oxy)-1,2-dihydro-3H-benzo[e]indol-3-yl)-2-oxoethyl)benzyl)oxy)-6-hydroxy-2-methoxy-12-oxo-6,6a,7,8,9,10-hexa-hydrobenzo[e]pyrido[1,2-a][1,4]diazepine-5(12H)-carboxylate (164)

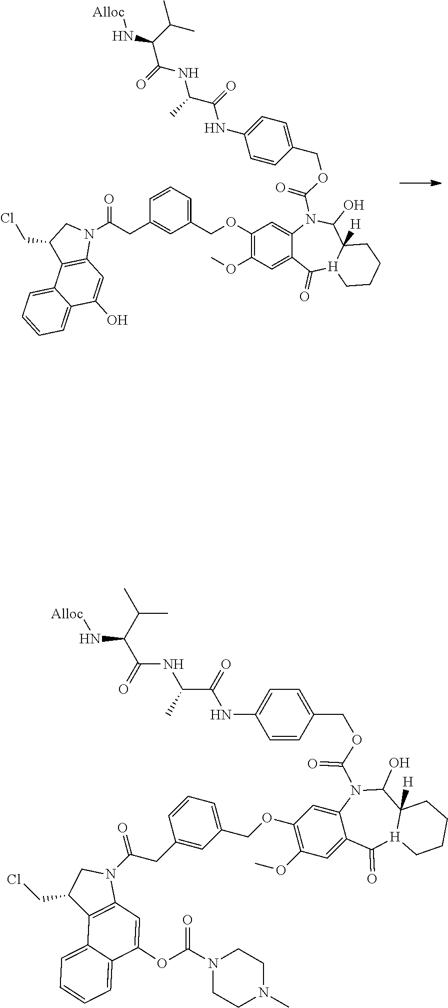

A solution of 4-((S)-2-((S)-2-(((allyloxy)carbonyl)amino)-3-methylbutanamido)-propanamido)benzyl (6aS)-3-((3-(2-((S)-1-(chloromethyl)-5-hydroxy-1,2-dihydro-3H-benzo [e]indol-3-yl)-2-oxoethyl)benzyl)oxy)-6-hydroxy-2-methoxy-12-oxo-6,6a,7,8,9,10-hexahydrobenzo[e]pyrido[1,2-a][1,4]diazepine-5(12H)-carboxylate (163) (153 mg, 0.146 mmol) in dichloromethane (2 mL) was charged with 4-methyl-1-piperazinecarbonyl chloride hydrochloride (29 mg, 0.146 mmol), 4-(dimethylamino)-pyridine (20 mg, 0.16 mmol) and triethylamine (71 μL, 0.51 mmol) and stirred at room temperature for 18 h. The reaction mixture was subsequently concentrated in vacuo, then charged with diethyl ether (10 mL) and concentrated again. Purification by flash column chromatography (silica), eluting with ethyl acetate (100%), then triethylamine/ethyl acetate (5%), then methanol/triethylamine/ethyl acetate (from 0:5:95 to 10:5:90), gave the title compound (133 mg, 78%) as a white solid.

¹H NMR (400 MHz, acetone-d₆) δ 9.42 (s, 1H), 8.40 (s, 1H), 7.92 (apparent t, J=7.3 Hz, 2H), 7.76 (s, 1H), 7.62 (d, J=8.3 Hz, 2H), 7.55 (t, J=70.6 Hz, 1H), 7.55 (t, J=70.6 Hz, 1H), 7.46-7.41 (m, 1H), 7.36-7.30 (m, 4H), 7.22 (d, J=7.3 Hz, 2H), 7.08 (s, 1H), 6.73 (s, 1H), 6.52 (d, J=7.4 Hz, 1H), 5.97 (d, J=10.2 Hz, 1H), 5.89 (ddd, J=15.9, 10.5, 5.3 Hz, 1H), 5.27 (d, J=17.2 Hz, 1H), 5.12 (d, J=10.6 Hz, 1H), 4.95-4.83 (m, 1H), 4.75 (d, J=11.5 Hz, 1H), 4.54-4.44 (m, 5H), 4.30-4.21 (m, 2H), 4.07-3.97 (m, 4H), 3.82 (br, 2H), 3.80 (s, 3H), 3.55 (br, 2H), 3.40-3.32 (m, 2H), 3.30 (s, 2H), 2.54-2.37 (m, 5H), 2.28 (s, 3H), 1.79-1.48 (m, 6H), 1.33 (d, J=6.7 Hz, 3H), 0.93 (dd, J=13.7, 7.0 Hz, 6H); MS (ES+): m/z=1171 (M+H)⁺; LCMS (Method A): t_{R}=6.33 min.

### Example 167: 4-((S)-2-((S)-2-Amino-3-methylbutanamido)propanamido)-benzyl (6aS)-3-((3-(2-((S)-1-(chloromethyl)-5-((4-methylpiperazine-1-carbonyl)oxy)-1,2-dihydro-3H-benzo[e]indol-3-yl)-2-oxoethyl)benzyl)oxy)-6-hydroxy-2-methoxy-12-oxo-6,6a,7,8,9,10-hexahydrobenzo[e]pyrido[1,2-a][1,4]diazepine-5(12H)-carboxylate (165)

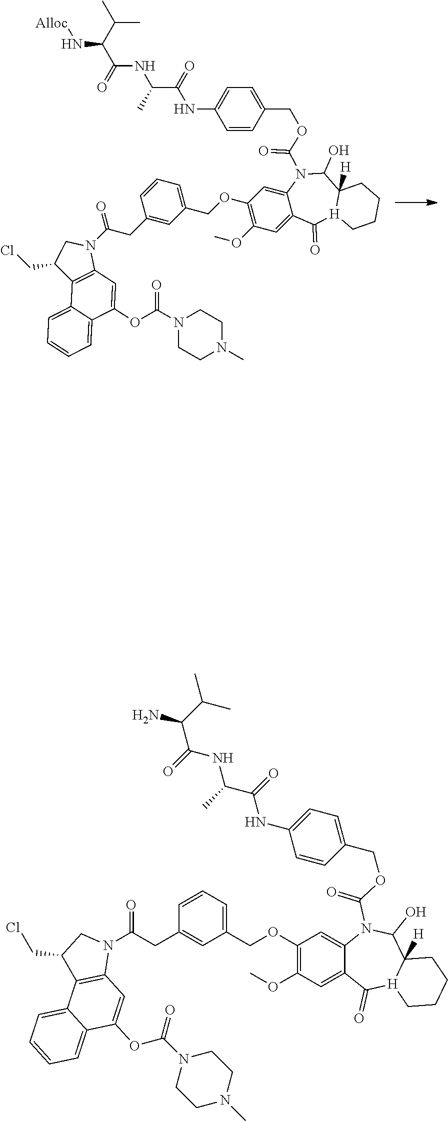

A solution of 4-((S)-2-((S)-2-(((allyloxy)carbonyl)amino)-3-methylbutanamido)propanamido)benzyl (6aS)-3-((3-(2-((S)-1-(chloromethyl)-5-((4-methylpiperazine-1-carbonyl)oxy)-1,2-dihydro-3H-benzo[e]indol-3-yl)-2-oxoethyl)benzyl)oxy)-6-hydroxy-2-methoxy-12-oxo-6,6a,7,8,9,10-hexahydrobenzo[e]pyrido[1,2-a][1,4]diazepine-5(12H)-carboxylate (164) (100 mg, 0.085 mmol) in dichloromethane (5 mL) was charged with pyrrolidine (8 μL, 0.094 mmol) and tetrakis(triphenylphosphine)palladium(0) (10 mg, 0.009 mmol) and the resulting mixture stirred for 40 min, whilst monitoring the reaction progress by LCMS. The mixture was then diluted into dichloromethane (15 mL) and filtered through a plug of celite. The filter cake was washed with dichloromethane (20 mL) and ethyl acetate (20 mL) and the combined filtrates concentrated in vacuo. The resulting residue was then charged with diethyl ether (20 mL) and concentrated again, then subjected to strong vacuum for 1 h, giving the title compound as a white solid, which was employed in the subsequent step immediately, without further purification.

¹H NMR (400 MHz, MeOD) δ 8.28 (s, 1H), 7.85 (d, J=90.2 Hz, 2H), 7.55 (d, J=70.8 Hz, 1H), 7.47 (s, 3H), 7.36-7.22 (m, 4H), 7.18-7.06 (m, 2H), 6.68 (s, 1H), 5.97-5.91 (m, 1H), 5.14 (br, 1H), 4.90 (br, 1H), 4.52-4.30 (m, 2H), 4.26-4.15 (m, 2H), 3.93 (d, J=90.1 Hz, 2H), 3.84 (dd, J=14.7, 7.4 Hz, 3H), 3.79 (s, 3H), 3.68-3.52 (m, 3H), 3.39-3.32 (m, 1H), 3.19-3.11 (m, 1H), 2.60-2.46 (m, 4H), 2.36 (s, 3H), 1.99 (dd, J=11.3, 3.8 Hz, 4H), 1.90 (d, J=6.0 Hz, 3H), 1.79-1.45 (m, 5H), 1.40-1.34 (m, 3H), 1.28 (br, 1H), 0.97-0.80 (m, 7H); MS (ES+): m/z=1087 (M+H)⁺; LCMS (Method A): t_{R}=5.45 min.

### Example 168: 4-((S)-2-((S)-2-(6-(2,5-Dioxo-2,5-dihydro-1H-pyrrol-1-yl)hexanamido)-3-methylbutanamido)propanamido)benzyl (6aS)-3-((3-(2-((S)-1-(chloromethyl)-5-((4-methylpiperazine-1-carbonyl)oxy)-1,2-dihydro-3H-benzo[e]indol-3-yl)-2-oxoethyl)benzyl)oxy)-6-hydroxy-2-methoxy-12-oxo-6,6a,7,8,9,10-hexahydrobenzo[e]pyrido[1,2-a][1,4]diazepine-5(12H)-carboxylate (166)

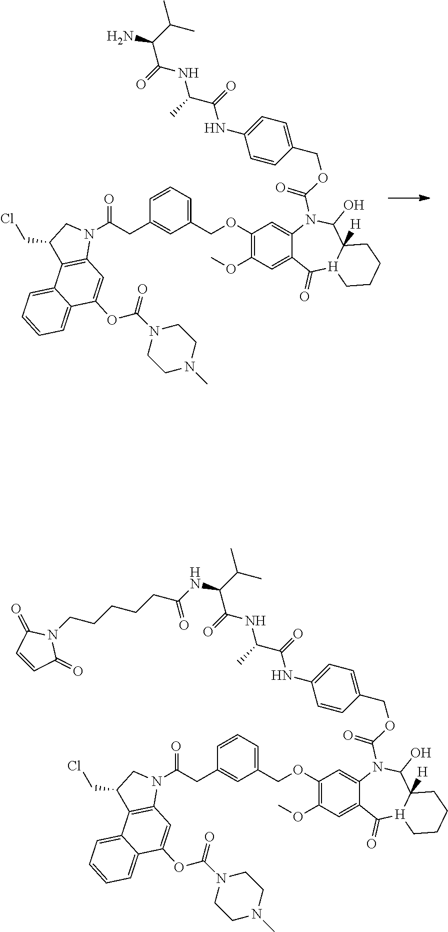

A solution of 4-((S)-2-((S)-2-amino-3-methylbutanamido)propanamido)benzyl (6aS)-3-((3-(2-((S)-1-(chloromethyl)-5-((4-methylpiperazine-1-carbonyl)oxy)-1,2-dihydro-3H-benzo [e]indol-3-yl)-2-oxoethyl)benzyl)oxy)-6-hydroxy-2-methoxy-12-oxo-6,6a,7,8,9,10-hexahydrobenzo[e]pyrido[1,2-a][1,4]diazepine-5(12H)-carboxylate (165) (93 mg, 0.085 mmol) in dichloromethane (10 mL) was charged with 6-maleimidohexanoic acid (18 mg, 0.085 mmol) and N-(3-dimethylaminopropyl)-N′-ethylcarbodiimide hydrochloride (16 mg, 0.085 mmol) and stirred at room temperature for 4 h. The resulting mixture was then concentrated partially in vacuo, and then loaded directly onto silica. Purification by flash column chromatography (silica), eluting with ethyl acetate (100%), then triethylamine/ethyl acetate (5%), then methanol/triethylamine/ethyl acetate (from 0:5:95 to 10:5:90), gave the title compound (66 mg, 61% over two steps) as a white solid.

¹H NMR (400 MHz, acetone-d₆) δ 9.38 (s, 1H), 8.37 (s, 1H), 7.91 (apparent t, J=70.5 Hz, 2H), 7.77 (s, 1H), 7.66 (d, J=8.4 Hz, 2H), 7.54 (t, J=7.7 Hz, 1H), 7.45-7.37 (m, 3H), 7.30 (br, 4H), 7.22 (d, J=7.7 Hz, 2H), 7.07 (s, 1H), 6.80 (s, 2H), 6.73 (s, 1H), 5.96 (d, J=10.2 Hz, 1H), 5.24 (d, J=11.5 Hz, 1H), 4.89 (d, J=12.9 Hz, 1H), 4.74 (d, J=12.1 Hz, 1H), 4.47 (br, 3H), 4.30-4.21 (m, 3H), 4.06-3.93 (m, 3H), 3.81 (br, 2H), 3.79 (s, 3H), 3.53 (br, 2H), 3.40 (t, J=7.1 Hz, 2H), 3.36 (br, 1H), 2.54-2.36 (m, 4H), 2.26 (s, 3H), 2.08 (s, 4H), 1.77-1.46 (m, 9H), 1.32 (d, J=6.4 Hz, 3H), 1.29-1.22 (m, 2H), 1.21-1.17 (m, 1H), 1.06-1.01 (m, 1H), 0.91 (dd, J=90.7, 7.0 Hz, 6H); ¹³C NMR (100 MHz, acetone-d₆) δ 173.5, 171.3, 170.9, 170.8, 168.5, 153.2, 151.2, 149.0, 148.2, 141.4, 139.0, 136.9, 135.1, 134.2, 131.7, 129.9, 129.2, 128.9, 128.6, 127.4, 126.1, 124.6, 123.0, 122.5, 119.2, 115.0, 81.9, 70.5, 66.7, 58.9, 56.0, 55.4, 54.7, 54.4, 53.2, 49.6, 46.9, 45.4, 41.8, 38.3, 37.2, 35.4, 30.4, 29.7, 26.2, 25.1, 23.1, 23.0, 18.8, 17.7; MS (ES+): m/z=1280 (M+H)⁺; LCMS (Method A): t_{R}=6.28 min;

### Example 16q: Allyl (6aS)-3-((3-(2-((S)-1-(chloromethyl)-5-((4-nitrobenzyl)-oxy)-1,2-dihydro-3H-benzo[e]indol-3-yl)-2-oxoethyl)benzyl)oxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (167)

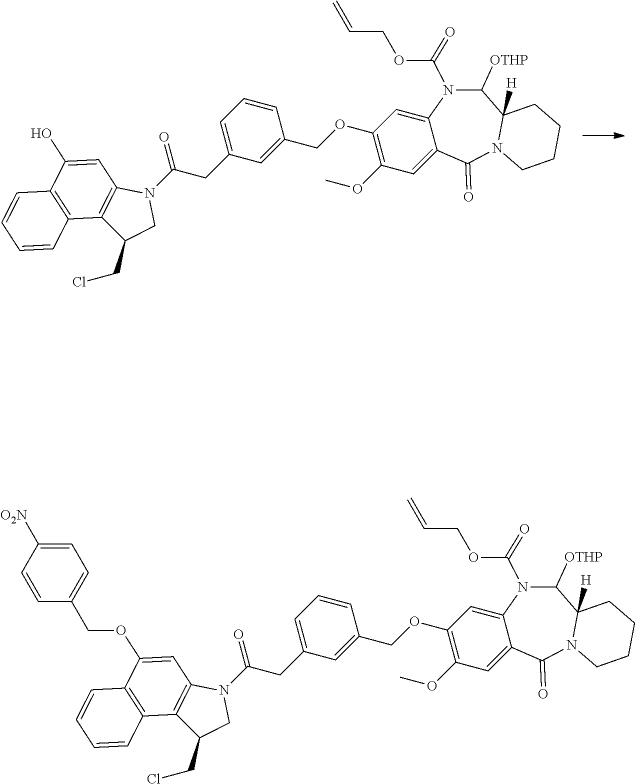

A solution of allyl (6aS)-3-((3-(2-((S)-1-(chloromethyl)-5-hydroxy-1,2-dihydro-3H-benzo[e]indol-3-yl)-2-oxoethyl)benzyl)oxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (87) (468 mg, 0.578 mmol) in N,N-dimethylformamide (2 mL) was charged with 4-nitrobenzyl bromide (151 mg, 0.700 mmol) and potassium carbonate (160 mg, 1.16 mmol) and the resulting mixture stirred at room temperature for 3 h. The mixture was then diluted into ethyl acetate (100 mL) and washed with cold brine (2×20 mL). After drying over magnesium sulfate, filtering and concentrating in vacuo, the residue was purified by flash column chromatography (silica), eluting with ethyl acetate/petroleum spirit, 40-60° C. (from 30% to 100%) to give the title compound (323 mg, 59%) as a yellow solid.

MS (ES+): m/z=945 (M+H)⁺; LCMS (Method A): t_{R}=9.75 min.

### Example 170: Allyl (6aS)-3-((3-(2-((S)-5-((4-aminobenzyl)oxy)-1-(chloro-methyl)-1,2-dihydro-3H-benzo[e]indol-3-yl)-2-oxoethyl)benzyl)oxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (168)

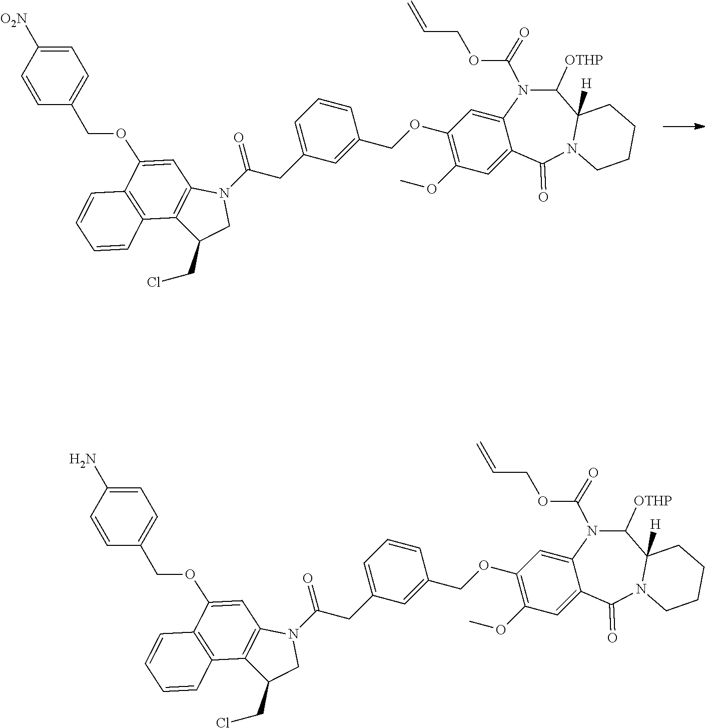

A solution of allyl (6aS)-3-((3-(2-((S)-1-(chloromethyl)-5-((4-nitrobenzyl)oxy)-1,2-dihydro-3H-benzo[e]indol-3-yl)-2-oxoethyl)benzyl)oxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (167) (61 mg, 0.065 mmol) in tetrahydrofuran (3 mL) and acetone (2 mL) was charged with water (1 mL), ammonium chloride (207 mg, 3.87 mmol) and zinc powder (127 mg, 1.94 mmol) and the resulting mixture stirred rapidly, at room temperature, under argon, for 1 h. The mixture was then filtered through a pad of celite and the filter cake washed with dichloromethane (100 mL) and water (100 mL). The resulting filtrate was separated, and the organic phase washed with brine (2×50 mL), dried over magnesium sulfate, filtered and concentrated in vacuo, to give a white solid, which was used in the subsequent step without further purification.

MS (ES+): m/z=915 (M+H)⁺; LCMS (Method A): t_{R}=8.78 min.

### Example 171: Allyl (6aS)-3-((3-(2-((S)-5-((4-((S)-2-((S)-2-(((allyloxy)-carbonyl)amino)-3-methylbutanamido)propanamido)benzyl)oxy)-1-(chloromethyl)-1,2-dihydro-3H-benzo[e]indol-3-yl)-2-oxoethyl)benzyl)-oxyl)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (169)

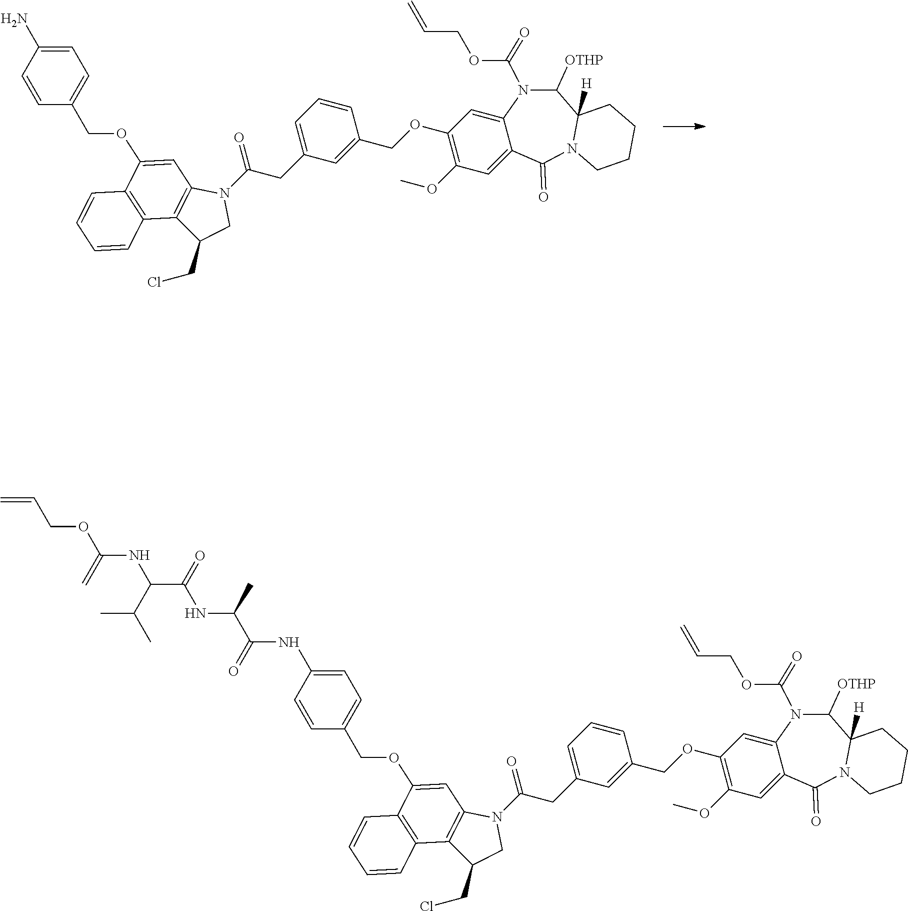

A solution of allyl (6aS)-3-((3-(2-((S)-5-((4-aminobenzyl)oxy)-1-(chloromethyl)-1,2-dihydro-3H-benzo[e]indol-3-yl)-2-oxoethyl)benzyl)oxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (168) (59 mg, 0.065 mmol) in dichloromethane (1 mL) was charged with ((allyloxy)carbonyl)-L-valyl-L-alanine (155) (18 mg, 0.065 mmol) and N-(3-dimethylaminopropyl)-N′-ethylcarbodiimide hydrochloride (12 mg, 0.065 mmol) and the resulting mixture stirred at room temperature for 16 h. The reaction mixture was subsequently concentrated in vacuo and then loaded directly onto silica, and purified by flash column chromatography, eluting with ethyl acetate/petroleum spirit, 40-60° C. (from 0% to 100%) to give the title compound (38 mg, 51% over two steps) as a cream solid.

¹H NMR (400 MHz, CDCl₃) δ 8.89 (d, J=14.6 Hz, 1H), 8.24 (d, J=8.1 Hz, 1H), 8.14 (s, 1H), 7.60 (apparent dd, J=16.5, 8.0 Hz, 3H), 7.51-7.44 (m, 1H), 7.44-7.38 (m, 3H), 7.37-7.28 (m, 5H), 7.15 (d, J=10.4 Hz, 2H), 6.90 (s, 1H), 6.14 (d, J=9.5 Hz, 1H), 5.98 (d, J=10.0 Hz, 1H), 5.94-5.78 (m, 1H), 5.71-5.55 (m, 1H), 5.33-4.90 (m, 8H), 4.76-4.61 (m, 1H), 4.59-4.47 (m, 4H), 4.33 (t, J=9.9 Hz, 2H), 4.19 (t, J=10.3 Hz, 1H), 4.05 (t, J=6.7 Hz, 1H), 3.98 (br, 1H), 3.91-3.86 (m, 3H), 3.84 (s, 3H), 3.83-3.79 (m, 1H), 3.64-3.42 (m, 2H), 3.36 (t, J=10.7 Hz, 1H), 3.04 (t, J=12.2 Hz, 1H), 2.17-2.06 (m, 1H), 1.99-1.89 (m, 2H), 1.82-1.56 (m, 7H), 1.53-1.34 (m, 6H), 1.00-0.89 (m, 6H); ¹³C NMR (100 MHz, CDCl₃) δ 171.8, 171.1, 169.4, 169.1, 155.8, 149.6, 149.3, 141.7, 137.7, 137.0, 136.9, 134.5, 132.4, 129.7, 129.1, 128.4, 128.3, 127.7, 126.3, 123.8, 123.6, 123.3, 122.0, 120.4, 120.1, 120.0, 118.0, 117.0, 115.5, 115.1, 100.3, 98.0, 88.1, 84.2, 70.0, 66.0, 64.3, 60.4, 56.1, 53.4, 49.5, 46.1, 43.4, 42.3, 38.8, 31.0, 30.6, 25.2, 23.2, 23.0, 21.0, 19.2, 19.1; MS (ES+): m/z=1169 (M+H)⁺; LCMS (Method A): t_{R}=9.20 min.

### Example 172: (S)-2-Amino-N—((S)-1-((4-((((S)-1-(chloromethyl)-3-(2-(3-((((S)-2-methoxy-12-oxo-6a,7,8,9,10,12-hexahydrobenzo[e]pyrido[1,2-a][1,4]diazepin-3-yl)oxy)methyl)phenyl)acetyl)-2,3-dihydro-1H-benzo[e]indol-5-yl)oxy)methyl)phenyl)amino)-1-oxopropan-2-yl)-3-methylbutanamide (170)

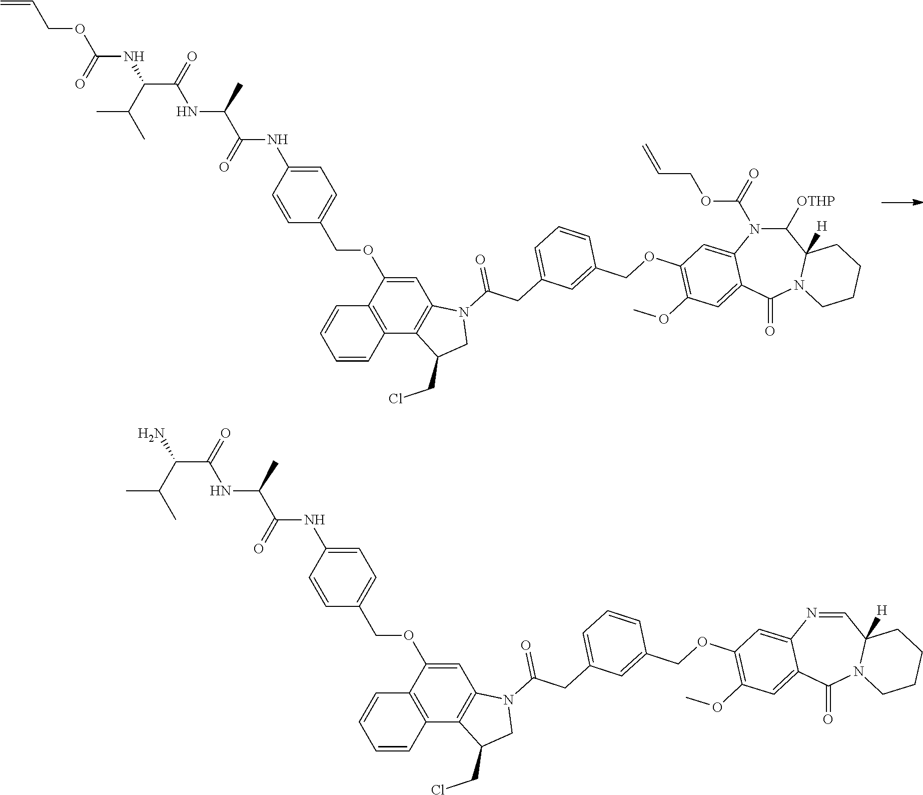

A solution of allyl (6aS)-3-((3-(2-((S)-5-((4-((S)-2-((S)-2-(((allyloxy)carbonyl)amino)-3-methylbutanamido)propanamido)benzyl)oxy)-1-(chloromethyl)-1,2-dihydro-3H-benzo[e]indol-3-yl)-2-oxoethyl)benzyl)oxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (169) (40 mg, 0.034 mmol) in dichloromethane (1 mL) was charged with tetrakis(triphenylphosphine)palladium(0) (4 mg), and pyrrolidine (6.3 μL, 0.075 mmol) and stirred at room temperature for 10 min. The resulting mixture was then diluted into dichloromethane (10 mL) and filtered through a pad of celite. The filter cake was then washed with dichloromethane (10 mL) and the filtrate concentrated in vacuo. Diethyl ether (10 mL) was charged to the residue and the resulting mixture concentrated in vacuo again. Strong vacuum was then applied to the residue for 20 min, before it was employed in the subsequent step without further purification.

MS (ES+): m/z=899 (M+H)⁺; LCMS (Method A): t_{R}=6.43 min.

### Example 173: N—((S)-1-(((S)-1-((4-((((S)-1-(Chloromethyl)-3-(2-(3-((((S)-2-methoxy-12-oxo-6a,7,8,9,10,12-hexahydrobenzo[e]pyrido[1,2-a][1,4]diazepin-3-yl)oxy)methyl)phenyl)acetyl)-2,3-dihydro-1H-benzo[e]indol-5-yl)oxy)methyl)phenyl)amino)-1-oxopropan-2-yl)amino)-3-methyl-1-oxobutan-2-yl)-6-(2,5-dioxo-2,5-dihydro-1-H-pyrrol-1-yl)hexanamide (171)

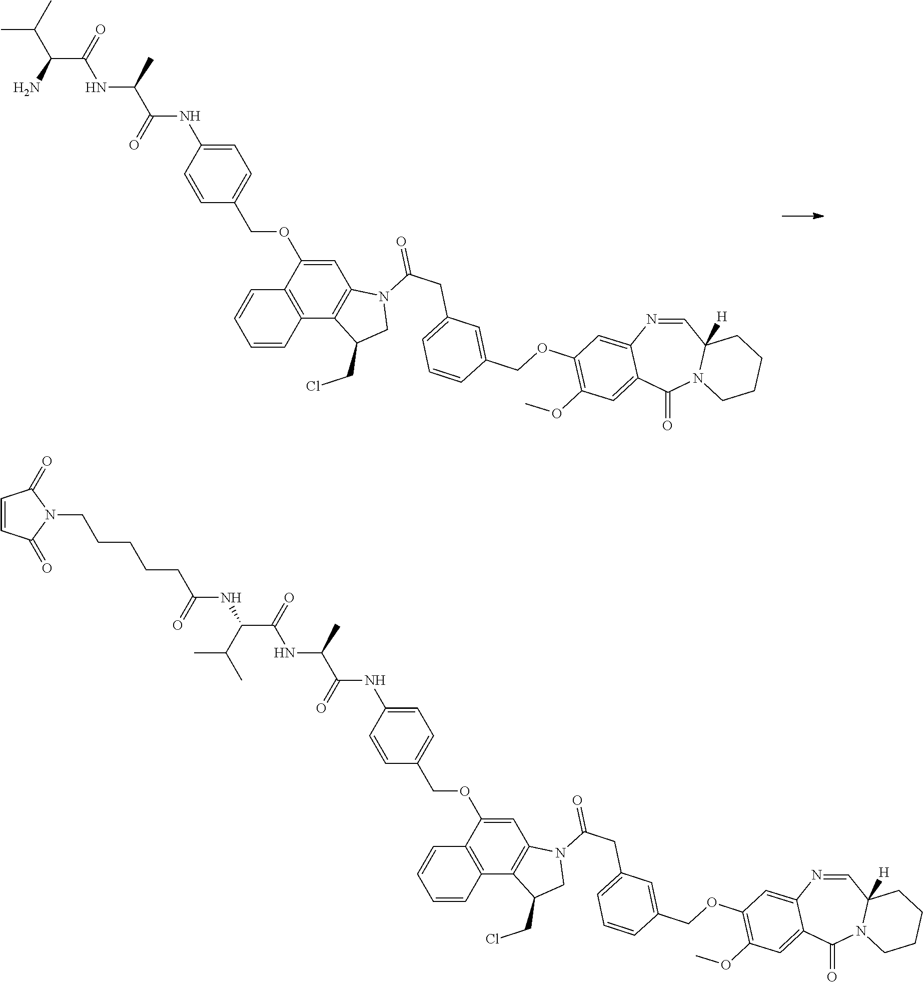 [SMILES: COc1cc2c(cc1OCc1cccc(CC(=O)N3C[C@@H](CCl)c4c3cc(OCc3ccc(NC(=O)[C@H](C)NC(=O)[C@@H](N)C(C)C)cc3)c3ccccc43)c1)N=C[C@@H]1CCCCN1C2=O ; COc1cc2c(cc1OCc1cccc(CC(=O)N3C[C@@H](CCl)c4c3cc(OCc3ccc(NC(=O)[C@H](C)NC(=O)[C@@H](NC(=O)CCCCCN5C(=O)C=CC5=O)C(C)C)cc3)c3ccccc43)c1)N=C[C@@H]1CCCCN1C2=O]

A solution of (S)-2-amino-N—((S)-1-((4-((((S)-1-(chloromethyl)-3-(2-(3-((((S)-2-methoxy-12-oxo-6a,7,8,9,10,12-hexahydrobenzo[e]pyrido[1,2-a][1,4]diazepin-3-yl)oxy)methyl)phenyl)acetyl)-2,3-dihydro-1H-benzo [e]indol-5-yl)oxy)methyl)phenyl)amino)-1-oxopropan-2-yl)-3-methylbutanamide (170) (31 mg, 0.034 mmol) in dichloromethane (1 mL) was charged with 6-maleimidohexanoic acid (7.1 mg, 0.034 mmol) and N-(3-dimethylaminopropyl)-N′-ethylcarbodiimide hydrochloride (6.6 mg, 0.034 mmol) and stirred at room temperature for 1.5 h. More 6-maleimidohexanoic acid (7.1 mg, 0.034 mmol) and N-(3-dimethylaminopropyl)-N′-ethylcarbodiimide hydrochloride (6.6 mg, 0.034 mmol) were added, and the mixture stirred for a further 2.5 h. The resulting mixture was then concentrated in vacuo, and then loaded directly onto silica. Purification by flash column chromatography, eluting with ethyl acetate/petroleum spirit, 40-60° C. (from 0% to 100%) then with methanol/ethyl acetate (from 0% to 20%) gave the title compound (16 mg, 43% over two steps) as a yellow solid.

¹H NMR (400 MHz, acetone-d₆) δ 9.30 (d, J=11.6 Hz, 1H), 8.23 (d, J=4.5 Hz, 2H), 7.90 (d, J=30.6 Hz, 2H), 7.80 (t, J=8.6 Hz, 2H), 7.70 (d, J=6.9 Hz, 1H), 7.55-7.45 (m, 5H), 7.43-7.29 (m, 6H), 6.84 (d, J=30.8 Hz, 1H), 6.78 (s, 1H), 6.72 (s, 1H), 5.25-5.17 (m, 4H), 4.59-4.51 (m, 1H), 4.47-4.33 (m, 2H), 4.22 (t, J=6.6 Hz, 1H), 4.18-4.09 (m, 2H), 4.01-3.91 (m, 4H), 3.83 (s, 3H), 3.75-3.65 (m, 3H), 3.12 (dd, J=17.4, 7.8 Hz, 1H), 2.32-2.21 (m, 3H), 1.84-1.74 (m, 4H), 1.67-1.54 (m, 7H), 1.33-1.26 (m, 3H), 0.97 (dd, J=6.9, 3.4 Hz, 6H); ¹³C NMR (100 MHz, DMSO-d₆) δ 171.7, 171.4, 171.3, 169.6, 166.7, 165.1, 164.2, 155.0, 150.4, 147.6, 142.4, 140.1, 136.9, 135.8, 134.8, 134.7, 130.1, 129.5, 128.8, 128.6, 124.0, 123.1, 122.6, 121.3, 119.7, 119.5, 116.0, 111.8, 110.4, 98.2, 70.4, 69.8, 56.0, 53.3, 49.7, 48.0, 47.9, 42.4, 41.2, 37.4, 37.3, 30.8, 28.2, 28.1, 26.2, 25.3, 24.1, 23.0, 18.1; MS (ES+): m/z=1092 (M+H)⁺; LCMS (Method A): t_{R}=7.90 min.

### Example 174: Biological and Biophysical Characterisation

Cytotoxicity in Cell Lines

The cytotoxicity of compounds 13, 24, 42, 53, 55, 57, 59 and 61 were evaluated in the FaDu (head and neck cancer) and PC3 (prostate) cell lines using the standard MTT assay for a 72 hour incubation period (Table 1). Some of the CBI-PDD dimers were extremely cytotoxic reaching IC₅₀ values of 10 picomolar in some cell lines (e.g., Compound 42 in FaDu). As anticipated, the prodrug forms of the molecules were significantly less active (e.g., Compound 59=4.5 micromolar in FaDu).

TABLE 1
Cytotoxicity of CBI-PDD analogues in the FaDu and PC3 cell lines.

| | Cytotoxicity | |
|---|---|---|
| Compound | FaDu | PC3 |
| Number | (nM, 72 hour) | (nM, 72 hour) |
| 13 | 1.17 | 1.43 |
| 24 | 1.83 | 0.12 |
| 42 | 0.01 | 0.03 |
| 53 | 0.65 | 0.02 |
| 55 | 1.5 | 0.02 |
| 57 | >10 | 3 |
| 59 | 4500 | 900 |
| 61 | >10 | >10 |

The cytotoxicity of a further series of compounds was evaluated in the SW48, LIM1215 and SW620 cell-lines (gastric) using the standard MTT assay for a 72 hour incubation period (Table 2). Some of the compounds were extremely cytotoxic reaching IC₅₀ values of 4 picomolar in some cell lines (e.g., compound 108 in SW48).

TABLE 2
Cytotoxicity of CBI-PDD analogues in the SW48,
LIM1215 and SW620 cell lines.

| | Cytotoxicity | | |
|---|---|---|---|
| Compound | SW48 | LIM1215 | SW620 |
| Number | (72 hrs) | (72 hours) | (72 hours) |
| 10 | 2916 | 1271 | 2002 |
| 83 | 4392 | 2890 | 3510 |
| 91 | 2317 | 2213 | 2612 |
| 99 | 0.035 | 0.039 | 0.045 |
| 13 | 0.28 | 0.68 | 0.42 |
| 57 | 0.042 | 0.41 | 1.1 |
| 108 | 0.004 | 0.78 | 0.045 |
| 139 | 0.95 | 12 | 13.5 |
| 140 | Not active | 18.1 | Not active |
| 143 | Not active | Not active | Not active |
| 145 | 32.1 | 136.9 | 350 |

Biophysical Characterisation

The ability of 13 to cross-link DNA was determined using an assay involving a linear double-stranded TyrT fragment (FIG. 8). The PBD dimer Talirine (SGD1882) was used as a positive control, as PBD dimers have previously been shown to cross-link DNA (33).

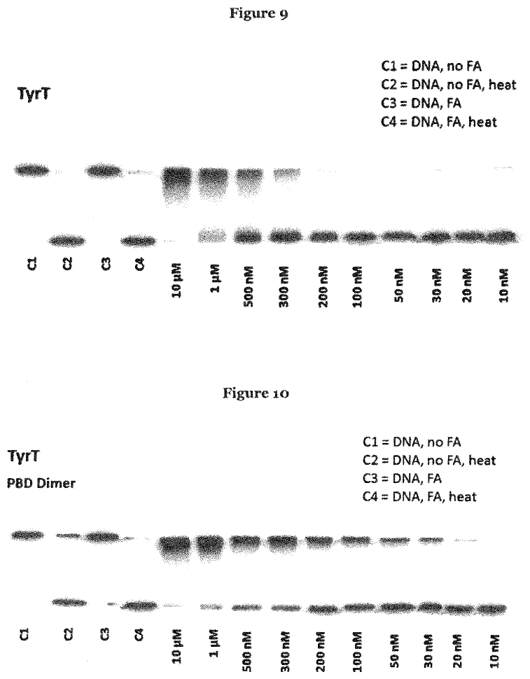

Following denaturation conditions (treatment with formamide and heating at 65° C. for 5 min) the DNA strands were completely separated (see controls C2 and C4, FIG. 9). The presence of an interstrand cross-link holds the denatured strands in close proximity, and cross-linked adducts therefore run as double-stranded DNA on polyacrylamide gel.

Each compound was tested at 10 different concentrations, and the assay was repeated twice. The cross-linking ability of 13 is shown in FIG. 9. Cross-links are clearly detectable at concentrations of 10 μM, 1 μM, 500 nM and 300 nM, and are visible at concentrations as low as 10 nM also. Using the same assay, the PBD dimer Talirine was also shown to cross-link DNA down to a concentration of 10 nM (FIG. 10). These results demonstrate that 13 can produce DNA cross-links at concentrations comparable to the PBD dimer.

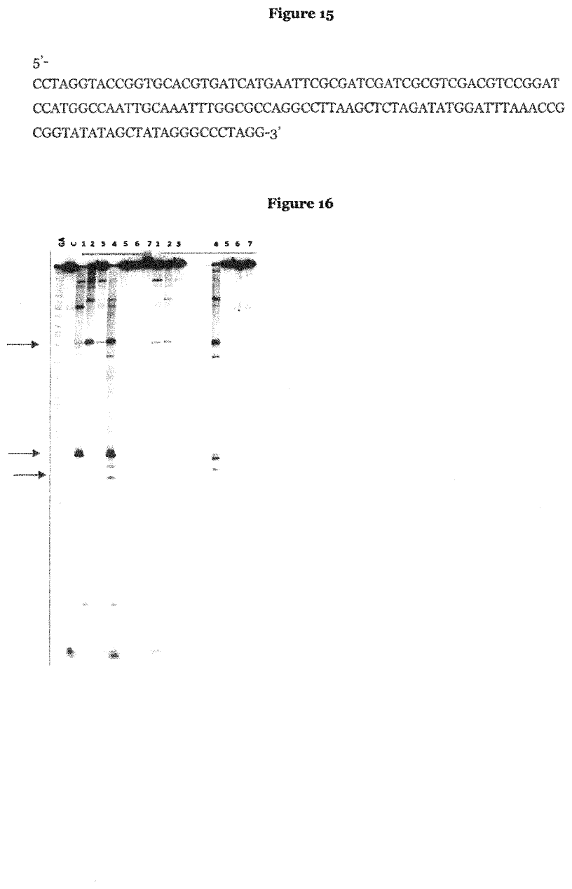

The ability of a further series of compounds 13, 42, 59, 99 and the PBD dimer Talirine to cross-link DNA was determined using an assay involving a linear double-stranded HexARev fragment (FIG. 15). The PBD dimer Talirine (SGD1882) (purchased from Aurum Pharmatech LLC) was used as a positive control, as PBD dimers have previously been shown to cross-link DNA (35).

The cleavage pattern of three molecules is evident in FIG. 15. Molecules 13, 42 and 99 (all containing active A-alkylating units) cleave DNA, whereas 59 (containing a carbamate protecting group on the A-alkylating unit) exhibits limited cleavage.

Cleavage Assay

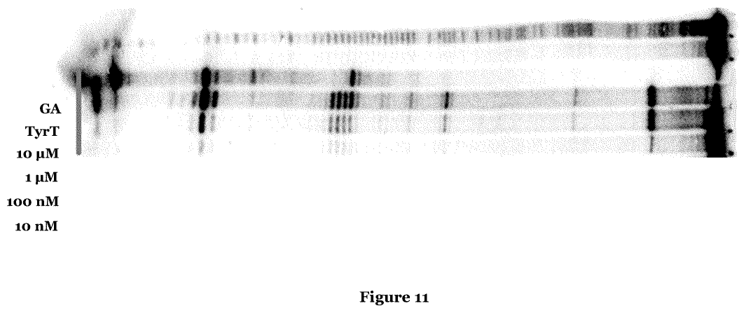

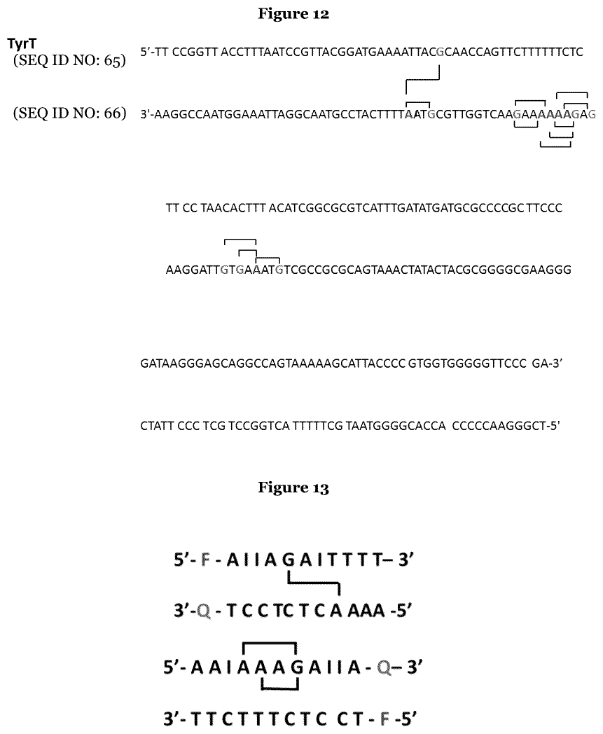

The ability of 13 and of a series of compounds to cleave double-stranded DNA was investigated using a modification of the previously established DNA footprinting assay (34). Following an overnight incubation of the ligand-DNA complexes, the mixture was mixed with strand separation buffer containing 10 mM EDTA, 10 mM NaOH, 0.1% bromophenol blue, 80% formamide and incubated at 100° C. for 3 min. The mixture was then immediately cooled on ice and run on an 8% denaturing gel. Examination of the obtained gel (FIG. 11) shows distinct cleavage patterns produced by 13 (black arrows). Using the GA marker, these additional bands were identified as cleavage products produced by 13 at certain A sites of the DNA fragment. Furthermore, the TyrT DNA fragment contains multiple potential binding sites for 13 (i.e., multiple examples of potential G-A cross-linking sites), but surprisingly only three preferred sites were observed during this experiment. This suggests that the molecule acts in a highly sequence selective manner. The possible adducts formed within the TyrT sequence are shown in FIG. 12.

Similarly, examination of the obtained gel (FIG. 16) shows distinct cleavage patterns produced by 77 (black arrows). Using the GA marker, these additional bands were identified as cleavage products produced by the series at certain A sites of the DNA fragment. Furthermore, the HexARev DNA fragment contains multiple potential binding sites for the series (i.e., multiple examples of potential G-A cross-linking sites), but surprisingly only a few preferred sites were observed during this experiment. This suggests that the molecules act in a highly sequence selective manner.

FRET DNA Melting

FRET DNA melting studies were undertaken on 13 using two fluorescently labelled sequences. The sequences (FIG. 13) were designed to provide additional evidence that 13 can form inter- and intrastrand cross-links. Inosines were inserted in place of traditional DNA bases to limit the number of binding sites available for 13 to interact with.

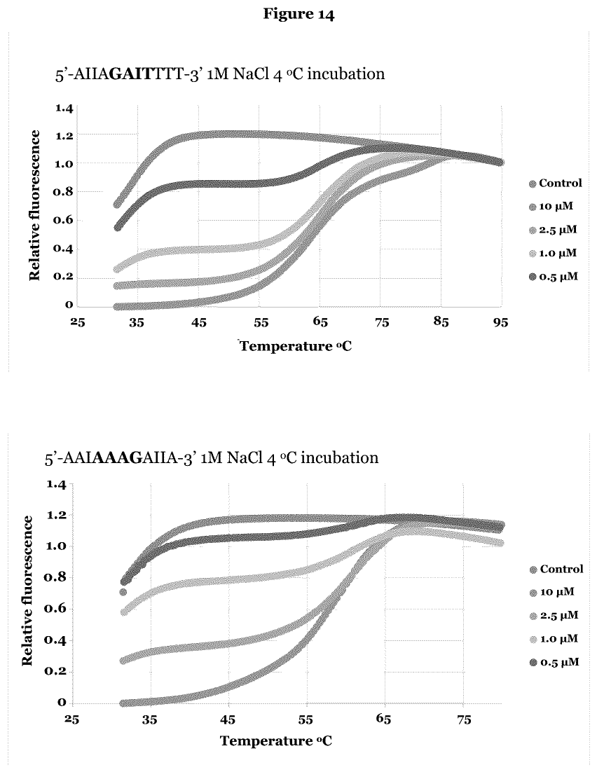

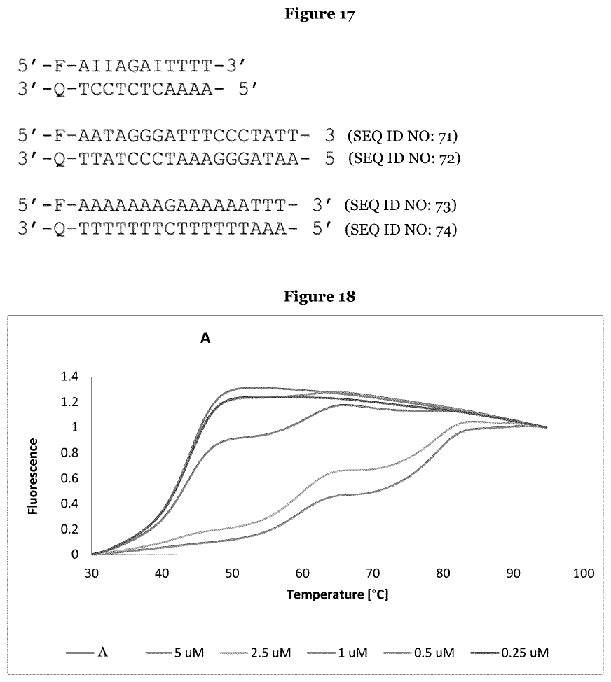

Further FRET DNA melting studies were undertaken on a number of molecules using three fluorescently labelled sequences. The sequences (FIG. 17) were designed to provide additional evidence that the series can form inter- and intrastrand cross-links. Inosines were inserted in place of traditional DNA bases in some cases to limit the number of binding sites available for the compounds to interact with. The short duplexes used in this FRET study are relatively unstable in the duplex form with a melting temperature below 30° C. so that, in the absence of ligand, a large part of the melting occurs below the starting temperature of the experiment. However, the inter- and intrastrand cross-links formed by 13 stabilizes the duplex form, producing very large increases in melting temperature with Tₘ values of ˜65° C. for 5′-AIIAGAITTTT-3′ (FIG. 14, top panel) suggesting interstrand cross-link formation and 60° C. for 5′-AAIAAAGAIIA-3′ (FIG. 14, bottom panel) suggesting intra-strand cross-link formation. Increasing concentrations of 13 cause a greater amount of the melting transition to appear at the higher temperature (i.e. a greater number of the DNA molecules are cross-linked). A greater effect for 5′-AIIAGAITTTI′-3′ can be observed at lower concentrations of 13. Furthermore, the interstrand cross-links formed by 42 stabilizes the duplex form, producing large increases in melting temperature with Tₘ values of ˜80° C. for 5′-AIIAGAITTTI-3′ and 5′-AATAGGGATTTCCCTATT-3′ (FIG. 17) suggesting interstrand cross-link formation.

Interestingly, the curves are biphasic, suggesting at least two cross-linked DNA adducts are formed in both sequences. As the sequences contain multiple G-A binding sites (e.g., 5′-GAIT-3′ in the top sequence in FIGS. 17 and 5′-GATT-3′ in the middle sequence in FIG. 17), this is an interesting observation.

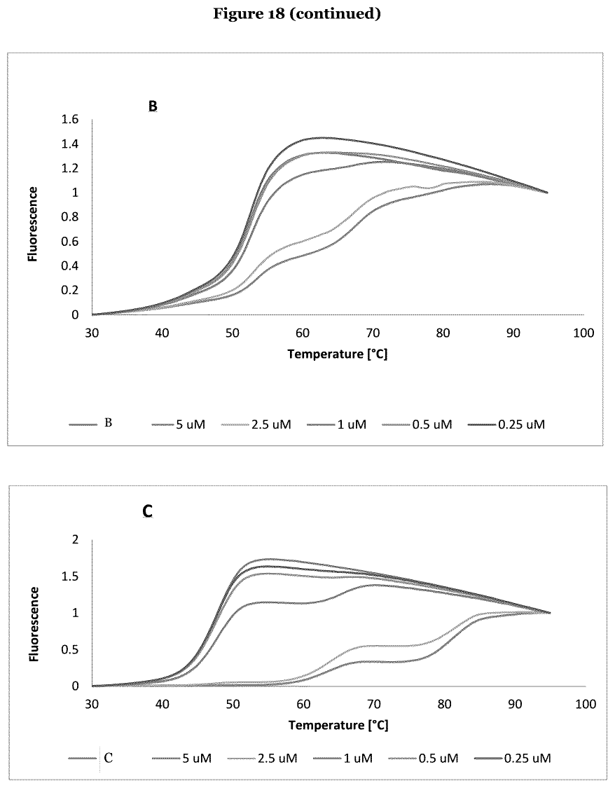

Compound 42 can also form intra-strand cross-links. This is evident in the bottom sequence in FIG. 17 (5′-AAAAAAAGAAAAAATIT-3′) where a Tₘ of ˜80° C. at higher concentrations is observed. In a similar manner to the top and middle sequences in FIG. 17, the curves for the FRET denaturation data shown in FIG. 18 is biphasic, suggesting more than one G-A cross-linked adduct. Analysis of the sequence indicates more than one binding site is present (e.g., 5′-GAAA-3′, 5′-GAAAA-3′ or 5′-AAAG-3′).

The melting temperature of each duplex increases significantly in proportion to the concentration of 42 present, providing strong supporting evidence that the compound can produce interstrand (FIGS. 18 A and B) and intrastrand (FIG. 18 C) cross-links.

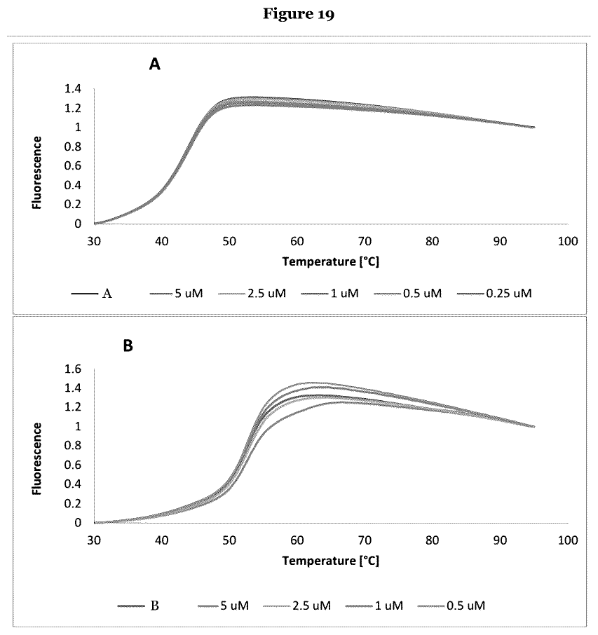

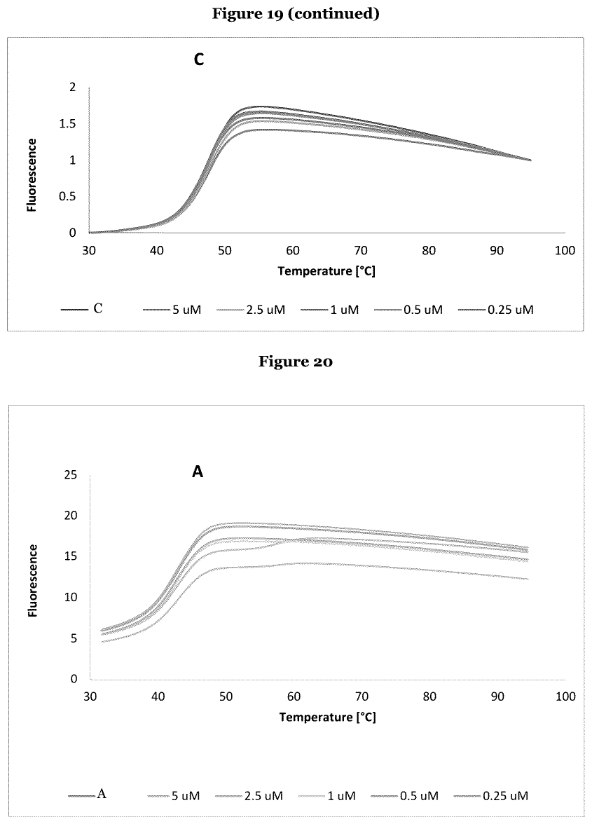

A number of other compounds were assessed to understand the contribution of each alkylating unit to the observed melting temperatures for the bis-alkylating molecules. Compound 59 (FIG. 19) does not impact on the stabilisation temperature of the parent DNA in the case of the three sequences shown in FIG. 17 suggesting that the presence of the carbamate protecting group both prevents the A-alkylating moiety from alkylating DNA, and inhibits the G-alkylating moiety from binding to DNA through steric interference.

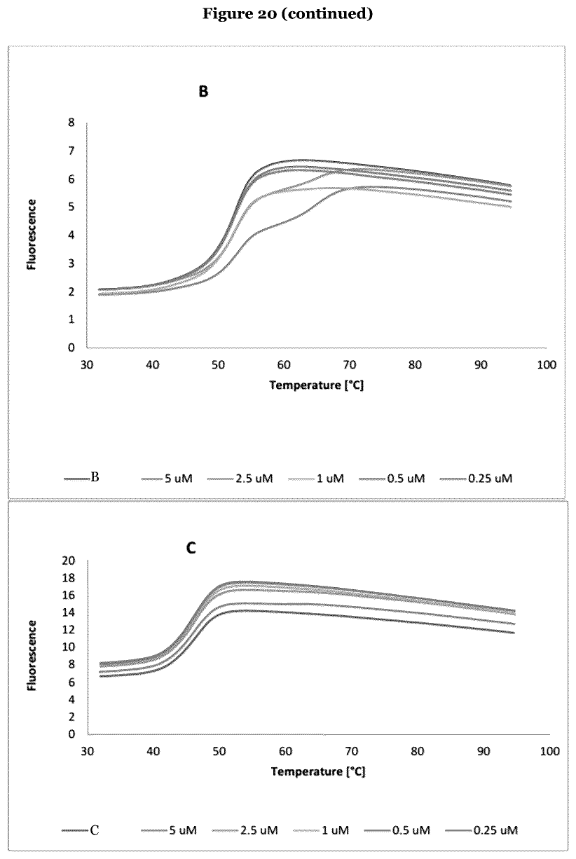

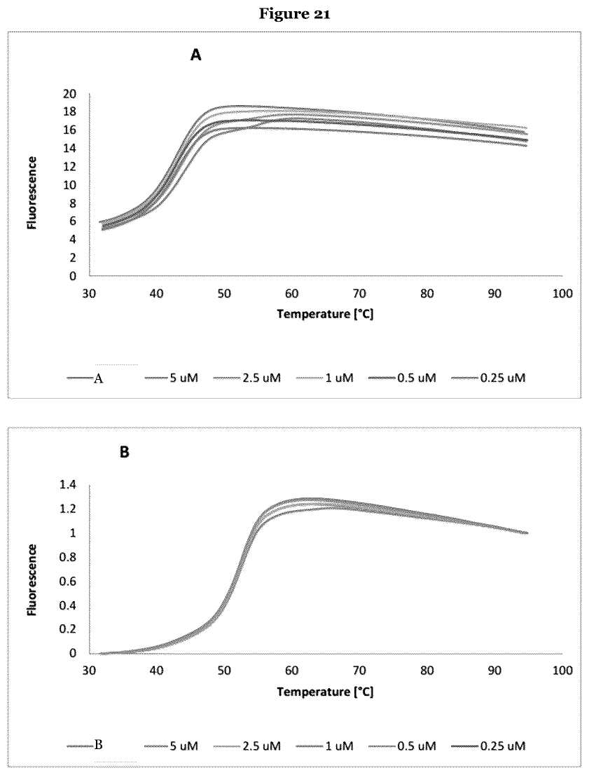

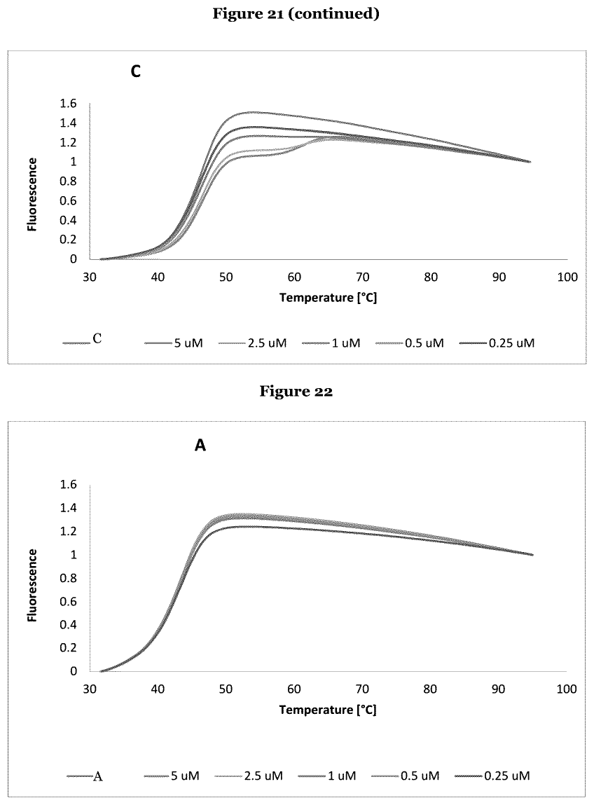

Compound 152 (FIG. 20) contains an active G-alkylating moiety and an inactive A-alkylating moiety. The relative contribution of the A-alkylating moiety to the potent DNA stabilisation observed in the G-A cross-linkers can be seen in the observed Tₘ values, where the ablation of A-alkylating ability results in a large decrease in the ΔTₘ value when compared to the active G-A cross-linker 42. Similarly, in the case of compound 149 (FIG. 21), removal of the ability of the compound to cross-link G-A sequences whilst retaining the ability to alkylate a guanine base results in a similar drop in ΔTₘ and change in profile of the fluorescence vs temperature curve when compared to the G-A cross-linker 42.

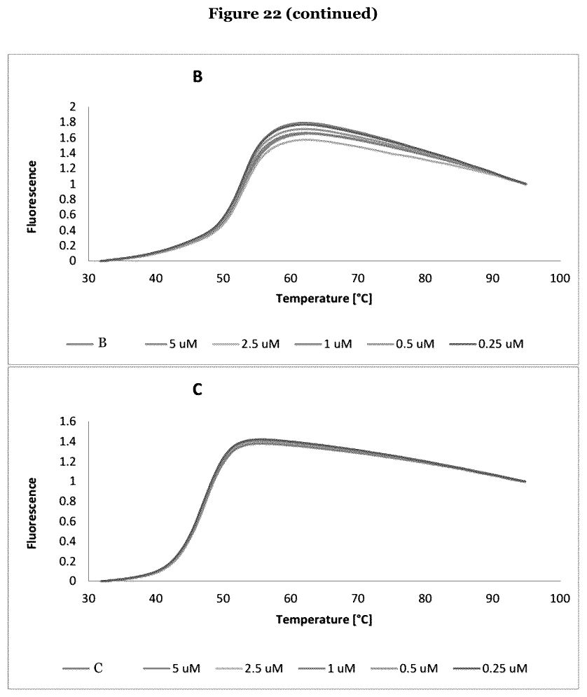

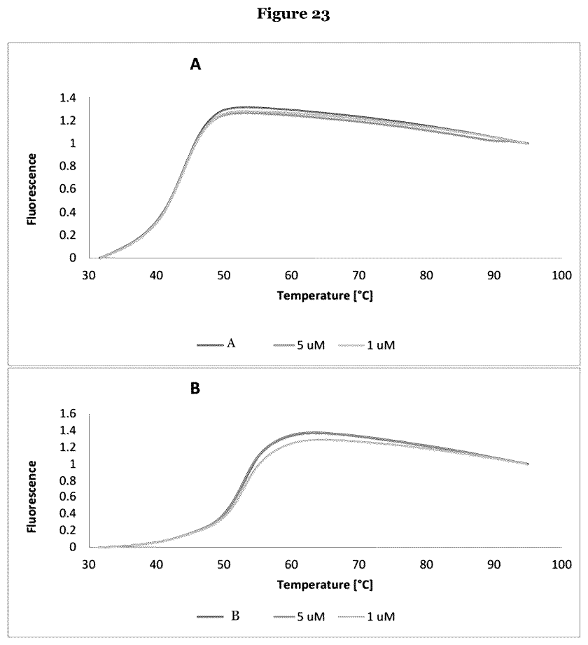

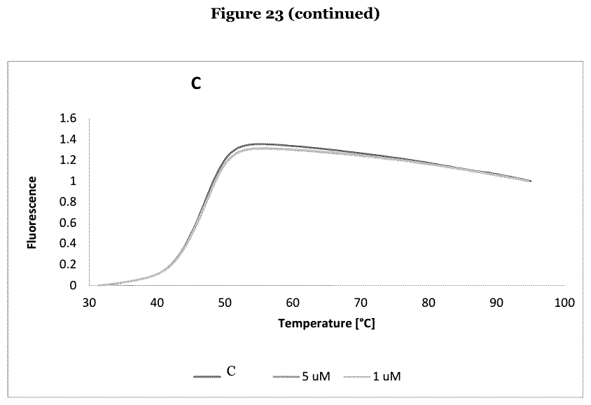

Finally, compounds 83 and 150 were also evaluated for ability to stabilise DNA. Compound 150 possesses a carbamate and inactive G-alkylating moiety, and was therefore expected to cause a very limited degree of DNA stabilisation (see FIG. 23), and compound 83 is a short A-alkylating unit (see FIG. 22). Compound 83 causes a very small degree of stabilisation in the case of the middle sequence and bottom sequence in FIG. 17 (both AT-rich) whereas 150 results in negligible DNA stabilisation.

Summary of Cross-Linking Data

Taken together, the cross-linking data presented above provide strong evidence that 13, 42 and 99 produce both intrastrand and interstrand cross-links which appear to form with a high degree of sequence-specificity (e.g., FIGS. 11 and 12). Furthermore, the importance of the contribution of both the G-alkylating and A-alkylating moieties to the stabilisation of DNA has been established, where the presence of both moieties results in a large degree of DNA stabilisation (and consequently potent cytotoxicity), whereas the absence/inhibition of binding of one moiety results in a large fall in DNA stabilising ability. It is possible that the compound may also form mono-alkylated adducts with guanine and adenine bases (see FIGS. 20 and 21). Together, this population of DNA adduct types may account for the cytotoxicity of this family of compounds in cells.

Conjugation

Stochastic Conjugation

Conjugation of 165 and 171 to IgG1 antibody (forming ADC1)

Compounds 165 and 171 were conjugated to an IgG1 antibody targeted to Antigen X in a stochastic manner.

Antibody QC

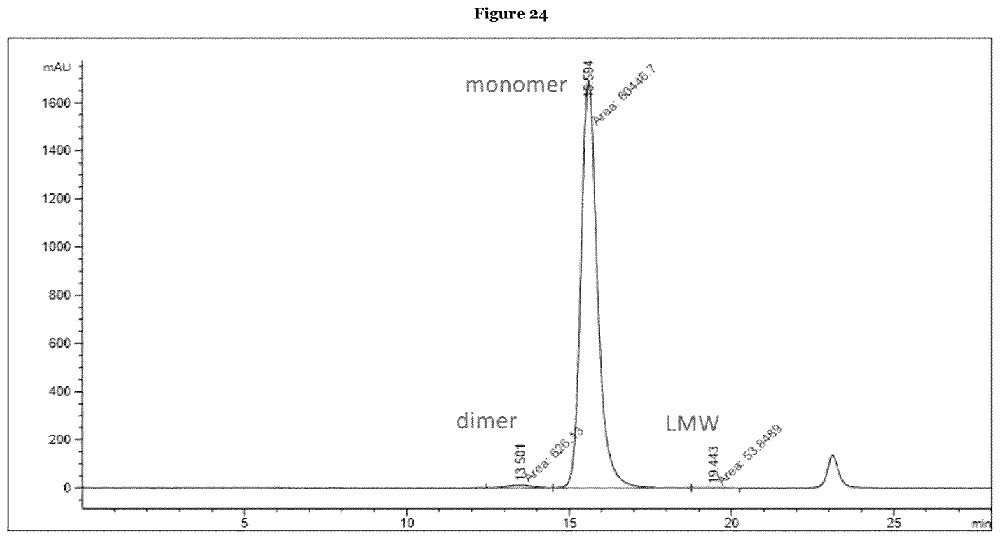

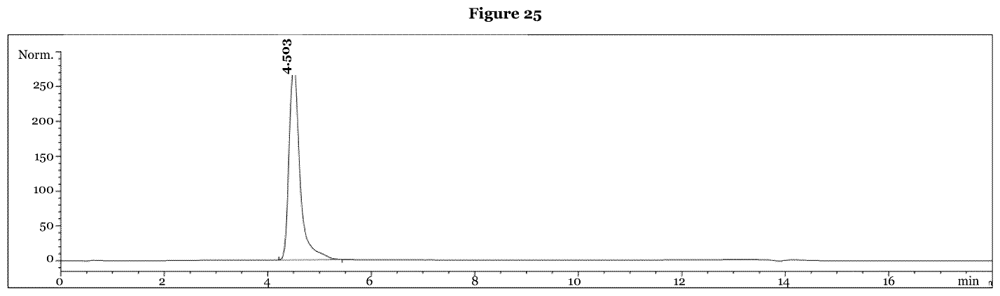

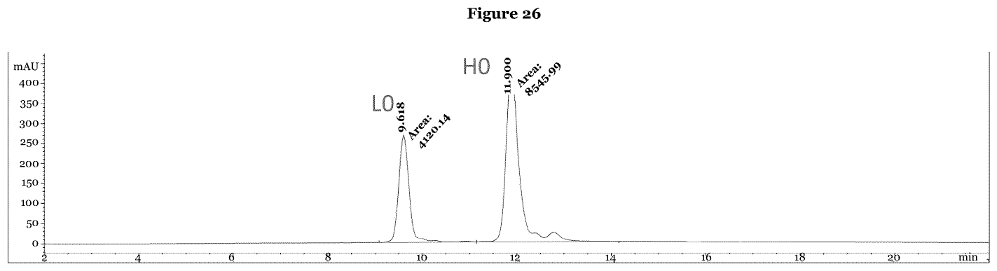

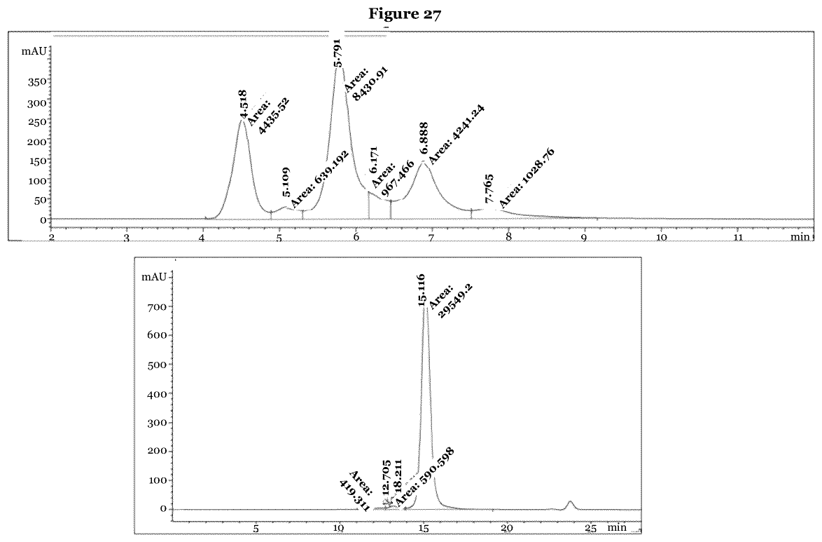

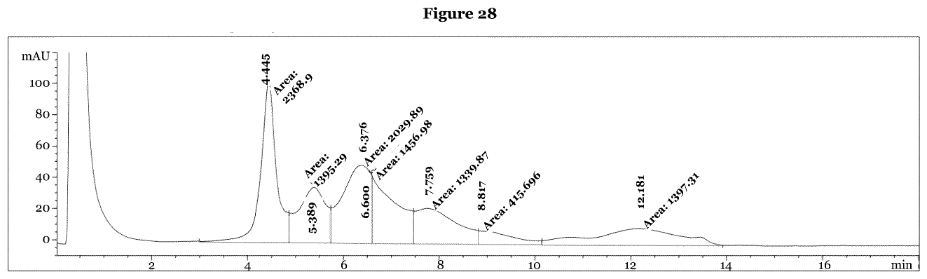

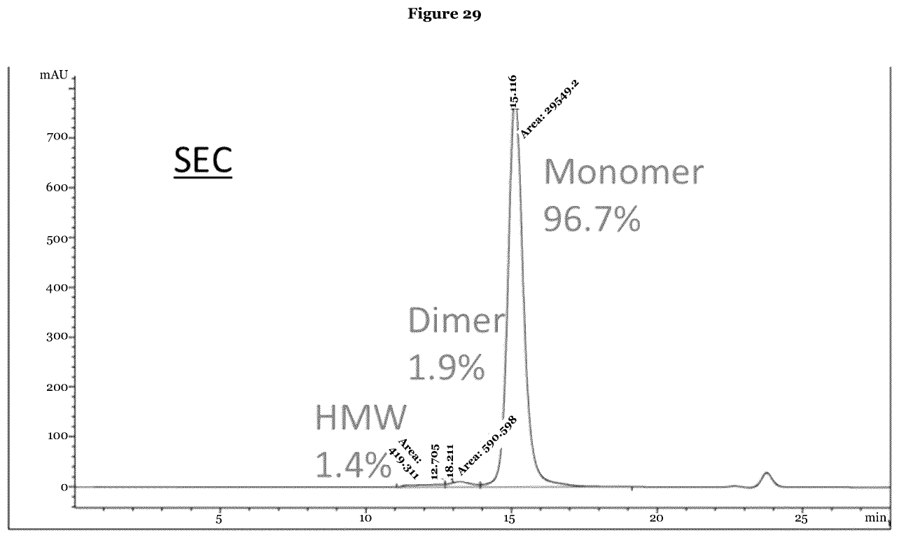

The antibody was of good quality with 98.9% monomer content (FIG. 24) and a single peak with a small shoulder on HIC (FIG. 25). PLRP showed the expected pattern for reduced Light and Heavy chain. The minor peaks eluting after the main Lo and Ho are likely the result of intrachain disulphide reduction (FIG. 26).

Conjugation of 165 and 171 to IgG1 Antibody

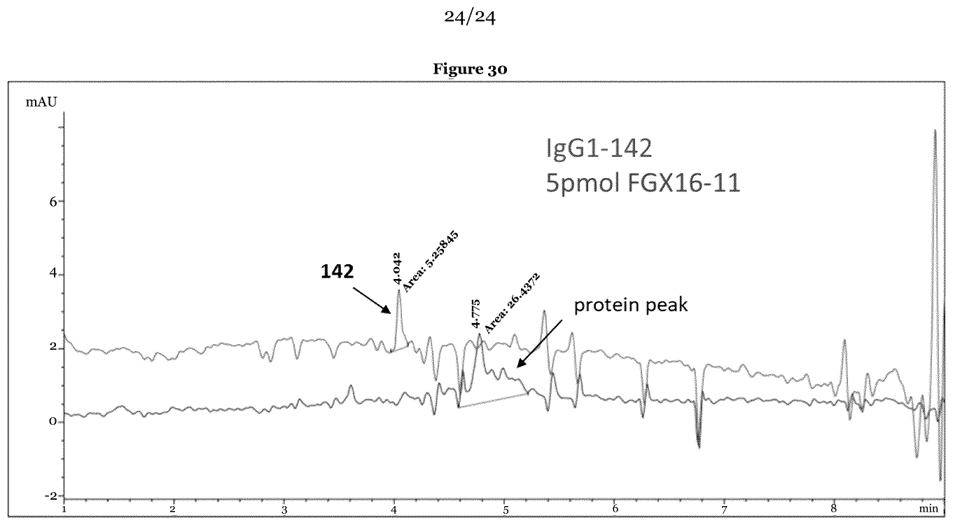

The conjugation process caused no significant aggregation compared to the starting antibody and contained 96.7% monomer in the case of 165. No free toxin linker could be detected in the ADC sample (see FIG. 30).

Biophysical Characterisation Methodology

1. Material

1.1. DNA Fragment

The preparation of the TyrT DNA fragment (FIG. 8) and HexARev DNA fragment (FIG. 15) have been previously described (34). Briefly, the sequence which had been cloned into the BamHI site of pUC18 was obtained by cutting with HindIII and EcoRI. Radiolabelled DNA fragments were prepared by filling in the 3′-end of the HindIII site with [α-³²P]dATP using Klenow DNA polymerase (exo-).

The radiolabelled DNA fragment was separated from the remainder of the plasmid DNA on a 6% non-denaturing polyacrylamide gel. The gel (20 cm long, 0.3 mm thick) was run at 400 V in 1×TBE running buffer for about 1-2 h, until the bromophenol blue had run most of the way down the gel. The glass plates were separated and the position of the labelled DNA fragment was established by short (1 min) exposure to an X-ray film. The relevant band was then cut from the gel and the radiolabelled DNA eluted by adding 300 μL 10 mM Tris-HCl, pH 7.5 containing 0.1 mM EDTA and gently agitating overnight at room temperature. The eluted DNA was finally precipitated with ethanol and re-suspended in a suitable volume of 10 mM Tris-HCl, pH 7.5 containing 0.1 mM EDTA buffer so as to give at least 10 counts per second/μL on a hand-held Geiger counter. With fresh plasmid and α-³²P-dATP this process typically generated about 150 μL of radiolabelled fragment DNA. The absolute concentration of the DNA is not important, and it is typically lower than 10 nM.

### 1.2. Compounds

Compounds such as 13 were synthesised as described above and the PBD dimer Talirine was obtained from Aurum Pharmatech LLC. Stock solution was prepared by dissolving the ligands in DMSO to give a concentration of 10 mM. From this stock solution, working solutions of the desired concentration were prepared by diluting with 10 mM Tris-HCl, pH 7.5 containing 10 mM NaCl.

2. Cleavage Assay

2.1. Preparation of Ligand-DNA Complexes

Radiolabelled DNA (1.5 μL) was mixed with 1.5 μL ligand solution of various concentrations (10 μM-10 nM) and incubated overnight at 37° C.

2.2. Preparation of GA Marker

Labelled DNA (1.5 μL) was mixed with 20 μL sterile water and 5 μL of denaturing loading solution (80% formamide containing 10 mM EDTA, 10 mM NaOH, 0.01% bromophenol blue). The sample was then incubated at 100° C. for 20 min with the micro-centrifuge tube cap open to allow evaporation.

2.3. Cleavage Assay

Loading solution (4.5 μL) was added to samples from Section 2.1. The digestion products were boiled for 3 min at 100° C. and quickly cooled on ice prior to electrophoresis. Separation was performed on an 8% denaturing polyacrylamide gel (40 cm long, 0.3 mm thick) at 1500V for about 2 h until the dye reached the bottom of the gel. The gel plates were then separated, the gels fixed by immersing in 10% (v/v) acetic acid, followed by transfer to Whatmann 3MM paper and drying under vacuum at 80° C. The dried gel was then exposed to a phosphorimager screen overnight before being scanned using a Typhon FLA 7000 instrument.

3. Cross-Linking Assay

3.1. Preparation of Ligand-DNA Complexes

Radiolabelled DNA (1.5 μL) was mixed with 1.5 μL ligand solution of various concentrations (10 μM-10 nM) and incubated overnight at 37° C.

3.2 Cross-Linking Assay

After overnight incubation, the samples were mixed with 7 μL loading solution (80% formamide containing 10 mM EDTA, 10 mM NaOH, 0.1% bromophenol blue) and incubated at 65° C. for 5 min. Control 1 (C1) for native double-stranded DNA consisted of 1.5 μL labelled DNA, 1.5 μL 10 mM Tris-HCl, pH 7.5 containing 0.1 mM EDTA and 7 μL 1× loading dye. Control 2 (C2) for denatured native single-stranded DNA was composed of 1.5 μL labelled DNA, 1.5 μL 10 mM Tris-HCl, pH 7.5 containing 0.1 mM EDTA which was incubated at 65° C. for 5 min. Control 3 (C3) for native double-stranded DNA consisted of 1.5 μL labelled DNA, 1.5 μL 10 mM Tris-HCl, pH 7.5 containing 0.1 mM EDTA and 7 μL SSB. Control 4 (C4) for denatured native single-stranded DNA was composed of 1.5 μL labelled DNA, 1.5 μL 10 mM Tris-HCl, pH 7.5 containing 0.1 mM EDTA and 7 μL SSB which was incubated at 65° C. for 5 min. Separation was performed on a 7.5% denaturing polyacrylamide gel (20 cm long, 0.3 mm thick) at 500V for about 4 h until the dye reached the bottom of the gel. The gel plates were then separated, the gels fixed by immersing in 10% (v/v) acetic acid, followed by transfer to Whatmann 3MM paper and drying under vacuum at 80° C. The dried gel was then exposed to a phosphorimager screen overnight before scanning using a Typhon FLA 7000 instrument.

FRET Studies Methodology

1. General

1.1. Oligonucleotides

Oligonucleotides were obtained from ATDbio (Southampton, UK) in lyophilised form. They were labelled with a fluorophore molecule (F=fluorescein) at the 5′-end and a quencher molecule (Q=dabcyl) at the 3′-end of the complementary strand. Each oligonucleotide was dissolved in distilled H₂O to form stock solutions of 100 PM. Working solutions of 5 μM were prepared by diluting the stock solution with distilled H₂O.

1.2. Buffers

The following buffers were used: 250 mM phosphate buffer pH 7.4 (consisting of sodium dihydrogen phosphate and sodium phosphate diluted in distilled H₂O) and 5 M sodium chloride buffer. A11 buffers and distilled H₂O were filtered through a 0.2 μM filter prior to use.

1.3. Compound

For the FRET experiments a stock solutions were prepared by dissolving compound (such as compound 13) in DMSO to give a concentration of 10 mM. From this stock solution, working solutions of the desired concentration were prepared by diluting the stock solution with distilled H₂O.

1.4. Preparation of Ligand-DNA Complexes

The reaction mixture was comprised of 4 μL of 250 mM phosphate buffer (final concentration of 50 mM), 4 μL flourophor and 4 μL quencher molecule of the appropriate oligonucleotide for a final concentration of 0.2 PM, 4 μL 5 M sodium chloride (final concentration of 1 M NaCl), and 4 μL of distilled H₂O. This mixture was heated in an Eppendorf tube at 90° C. for 1 min and slowly cooled down to room temperature. This process was carried out to anneal the single strands to double-stranded DNA. Following this, 4 μL of the ligand was added in the desired concentration and the mixture incubated overnight either at room temperature or 4° C. A control sample of DNA only was prepared by mixing 4 μL 250 mM phosphate buffer (final concentration of 50 mM) with 4 μL fluorophore-labelled and 4 μL quencher-labelled oligonucleotides (of the appropriate sequence) to give a final concentration of 0.2 PM, 4 μL 5 M sodium chloride (final concentration of 1 M NaCl) and 4 μL distilled H₂O. This mixture was analysed without prior annealing.

1.5. Fluorescence Melting

Fluorescence melting profiles were measured using a Roche LightCycler using a total reaction volume of 20 μL. Initially, the samples were denatured by heating to 95° C. at a rate of 1° C. min⁻¹. The samples were then maintained at 95° C. for 5 min before annealing by cooling to 25° C. at 1° C. min⁻¹. The samples were then held at 25° C. for a further 5 min and finally melted by heating to 95° C. at 1° C. min⁻¹. Annealing steps and melting steps were all recorded and changes in fluorescence were measured at 520 nm.

1.6. Data Analysis

Tₘ values were obtained from the first derivates of the melting profiles using the Roche LightCycler software.

MTT Cytotoxicity Methodology

Tumor cell lines were maintained in RPMI1640 medium supplemented with 10% heat-inactivated fetal bovine serum, 2 mM L-glutamine and 1 mM sodium pyruvate. 1800 cells per well were seeded in a volume of 180 μl in a 96-well flat bottom polystyrene plate. The cells were allowed to adhere overnight at 37° C. in a CO₂ incubator. Ligands were initially formulated in DMSO, and stocks stored at −80° C. They were then further formulated at lox concentration in RPMI1640 medium. 20 ul of diluted samples were added into each treatment well. On each plate, blank wells with no cells, and untreated wells containing cells, were included. Plates were then cultured at 37° C. in a CO₂ incubator for 72 hrs. Cytotoxicity was evaluated using a tetrazolium salt-based assay, the MTT assay. After 72 hours, the supernatant was removed from each well and 200 μl of a sterile filtered 500 μg/ml MTT solution in water added to each well. The plates were then incubated at 37° C. in a CO₂ incubator for 4 hrs. The supernatant was then removed and the formazan crystals formed solubilized by adding 150 μl of DMSO to each well. The plate was then read on a plate reader at 540 nm, and percentage cell survival calculated as follows: ((mean absorbance treated wells at concentration x−mean absorbance blank wells)±(mean absorbance untreated wells at concentration x−mean absorbance blank wells))×100. Data were plotted as concentration in nM vs. % cell survival in Microsoft Excel, and IC₅₀ values (concentration where cell survival is reduced by a half) were determined from the graph.

Conjugation Methodology

All ADC conjugations were completed using a similar methodology, an example of which is provided below. 21.5 mg IgG1 antibody (8.0 mg/ml in PBS) were charged with EDTA to a final concentration of 2 mM. Reduction was attained by adding 1.27 molar equivalents TCEP (10 mM in water) and incubating for 2 hours at 20° C. After 1.5 hours, a reduction in-process test conjugation with Mal-vcMMAE was performed, and analyzed by HIC to test for the reduction level. As the target reduction level had not been reached, another 0.1 molar equivalents TCEP were added and the reduction time extended by 1 hour. After 0.5 hours, a second in-process test was run. After confirmation of the desired reduction level, 20% (v/v) Propylene glycol was added to the reduced antibody followed by 6.4 molar equivalents 165/171 (10 mM stock in DMSO). The solution was incubated for 1 hour at rt. The reaction was quenched by adding 6.4 molar equivalents N-Acetylcysteine (10 mM in water). The ADC was buffer exchanged via G25 into PBS and washed by dead-end filtration (Vivaspin-20, 30 kDa MWCO, 0.0006 m²) for 10 DVs. Samples were taken for analysis by HIC, SEC, PLRP, free toxin linker, Endosafe, and the concentration was determined using a SEC calibration curve. Aliquotting was carried out under laminar flow, and the product was stored at −80° C. Only disposable, sterile and pyrogen/DNA/RNA-free plasticware was used.

### REFERENCES

1. Antonow, D., and Thurston, D. E. (2011) Chem Rev 111, 2815-2864.
2. Cipolla, L., Araujo, A. C., Airoldi, C., and Bini, D. (2009) Anticancer Agents Med Chem 9, 1-31.
3. Gerratana, B. (2012) Med Res Rev 32, 254-293.
4. Hartley, J. A. (2011) Expert Opin Investig Drugs 20, 733-744.
5. Kamal, A., Reddy, K. L., Devaiah, V., Shankaraiah, N., and Reddy, D. R. (2006) Mini Rev Med Chem 6, 53-69.
6. Hurley, L. H., Reck, T., Thurston, D. E., Langley, D. R., Holden, K. G., Hertzberg, R. P., Hoover, J. R., Gallagher, G., Jr., Faucette, L. F., Mong, S. M., (1988) Chem Res Toxicol 1, 258-268.
7. Wells, G., Martin, C. R., Howard, P. W., Sands, Z. A., Laughton, C. A., Tiberghien, A., Woo, C. K., Masterson, L. A., Stephenson, M. J., Hartley, J. A., Jenkins, T. C., Shnyder, S. D., Loadman, P. M., Waring, M. J., and Thurston, D. E. (2006) J Med Chem 49, 5442-5461.
8. Brucoli, F., Hawkins, R. M., James, C. H., Jackson, P. J., Wells, G., Jenkins, T. C., Ellis, T., Kotecha, M., Hochhauser, D., Hartley, J. A., Howard, P. W., and Thurston, D. E. (2013) J Med Chem 56, 6339-6351.
9. Kotecha, M., Kluza, J., Wells, G., O'Hare, C. C., Forni, C., Mantovani, R., Howard, P. W., Morris, P., Thurston, D. E., Hartley, J. A., and Hochhauser, D. (2008) Mol Cancer Ther 7, 1319-1328.
10. Puvvada, M. S., Hartley, J. A., Jenkins, T. C., and Thurston, D. E. (1993) Nucleic Acids Res 21, 3671-3675.
11. Clingen, P. H., De Silva, I. U., McHugh, P. J., Ghadessy, F. J., Tilby, M. J., Thurston, D. E., and Hartley, J. A. (2005) Nucleic Acids Res 33, 3283-3291.
12. Puvvada, M. S., Forrow, S. A., Hartley, J. A., Stephenson, P., Gibson, I., Jenkins, T. C., and Thurston, D. E. (1997) Biochemistry 36, 2478-2484.
13. Barkley, M. D., Cheatham, S., Thurston, D. E., and Hurley, L. H. (1986) Biochemistry 25, 3021-3031.
14. Seifert, J., Pezeshki, S., Kamal, A., and Weisz, K. (2012) Organic & Biomolecular Chemistry 10, 6850-6860.
15. Smellie, M., Bose, D. S., Thompson, A. S., Jenkins, T. C., Hartley, J. A., and Thurston, D. E. (2003) Biochemistry 42, 8232-8239.
16. Kopka, M. L., Goodsell, D. S., Baikalov, I., Grzeskowiak, K., Cascio, D., and Dickerson, R. E. (1994) Biochemistry 33, 13593-13610.
17. Kizu, R., Draves, P. H., and Hurley, L. H. (1993) Biochemistry 32, 8712-8722.
18. Wilkinson, G. P., Loadman, P. M., Taylor, J. P., Jenkins, T. C., Double, J. A., Gregson, S. J., Howard, P. W., and Thurston, D. E. (2002) European Journal of Cancer 38, S28-S29.
19. Schwartz, G. H., Patnaik, A., Hammond, L. A., Rizzo, J., Berg, K., Von Hoff, D. D., and Rowinsky, E. K. (2003)Annals of Oncology 14, 775-782.
20. Zhou, Q., Duan, W. H., Simmons, D., Shayo, Y., Raymond, M. A., Dorr, R. T., and Hurley, L. H. (2001) J. Am. Chem. Soc. 123, 4865-4866.
21. Tercel, M., Stribbling, S. M., Sheppard, H., Sum, B. G., Wu, K., Pullen, S. M., Botting, K. J., Wilson, W. R., and Denny, W. A. (2003) J. Med. Chem. 46, 2132-2151.
22. Purnell, B., Sato, A., O'Kelley, A., Price, C., Summerville, K., Hudson, S., O'Hare, C., Kiakos, K., Asao, T., Lee, M., and Hartley, J. A. (2006) Bioorganic & Medicinal Chemistry Letters 16, 5677-5681.
23. T. A. Halgren, (1996) Journal of Computational Chemistry, 17, 490-519.
24. D. A. Case, Darden, T. A., Cheatham III, T. E., Simmerling, C. L., Wang, J., Duke, R. E., Luo, R., Walker, R. C., Zhang, W., Merz, K. M., Roberts, B., Wang, B., Hayik, S., Roitberg, A., Seabra, G., Kolossváry, I., Wong, K. F., Paesani, F., Vanicek, J., Liu, J., Wu, X., Brozell, S. R., Steinbrecher, T., Gohlke, H., Cai, Q., Ye, X., Wang, J., Hsieh, M.-J., Cui, G., Roe, D. R., Mathews, D. H., Seetin, M. G., Sagui, C., Babin, V., Luchko, T., Gusarov, S., Kovalenko, A., Kollman, P. A., AMBER 11, University of California, San Francisco, 2010, 2010.
25. A. Perez, I. Marchan, D. Svozil, J. Sponer, T. E. Cheatham, 3rd, C. A. Laughton and M. Orozco, (2007) Biophys J, 92, 3817-3829.
26. S. N. Rao, U. C. Singh and P. A. Kollman, (1986) Journal of Medicinal Chemistry, 29, 2484-2492.
27. V. Tsui and D. A. Case, (2000) Biopolymers, 56, 275-291.
28. F. Brucoli, R. M. Hawkins, C. H. James, P. J. Jackson, G. Wells, T. C. Jenkins, T. Ellis, M. Kotecha, D. Hochhauser, J. A. Hartley, P. W. Howard and D. E. Thurston, (2013) Journal of Medicinal Chemistry, 56, 6339-6351.
29. K. M. Rahman, P. J. M. Jackson, C. H. James, B. P. Basu, J. A. Hartley, M. de la Fuente, A. Schatzlein, M. Robson, R. B. Pedley, C. Pepper, K. R. Fox, P. W. Howard and D. E. Thurston, (2013) Journal of Medicinal Chemistry, 56, 2911-2935.
30. P. J. Jackson, C. H. James, T. C. Jenkins, K. M. Rahman and D. E. Thurston, (2014) ACS Chem Biol, 9, 2432-2440.
31. G. Jai, J. W. Lown, (2000) Bioorganic & Medicinal Chemistry, 8, 1607-1617.
32. R. C. Elgersma, et al., (2015) Mol. Pharmaceutics, 12, 1813-1835.
33. K. M. Rahman, A. S. Thompson, C. H. James, M. Narayanaswamy, D. E. Thurston, Journal of the American Chemical Society 2009, 131, 13756-13766.
34. Drew, H. R. and A. A. Travers, DNA structural variations in the E. coli tyrT promoter. Cell, 1984. 37 (2): p. 491-502.
35. Hampshire, A. J., Rusling, D. A., Broughton-Head, V. J., and Fox, K. R. (2007) Methods 42, 128-140.

What is claimed:

1. A method of treatment of a patient suffering from a proliferative disease, comprising administering to said patient a therapeutically effective amount of an antibody-drug conjugate, wherein the drug is a compound of formula (I):
A-X₁-L-X₂-B (I)
and salts, solvates and tautomers thereof,
wherein;
A is a group selected from: 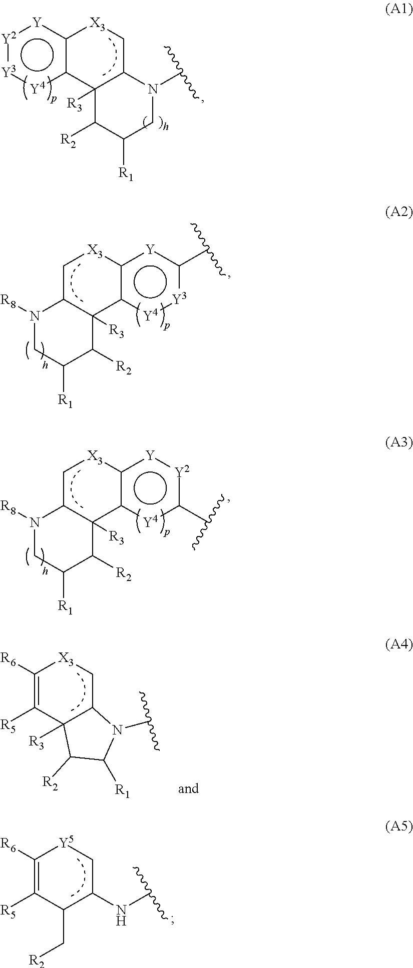
h is 0 or 1;
R₁ is selected from H and halogen;
either R₂ is selected from —CH₂-halogen, C₁₋₆ alkyl and H, and R₃ is H; or R₂ and R₃ together with the carbon atoms to which they are attached form a cyclopropyl ring;
p is 0 or 1; and when p is 1 then Y is C—R₇, Y² is C—R₆, Y³ is C—R₅ and Y⁴ is C—R₄; and for (A1) and (A2) when p is 0 either (a) Y is selected from N—R₁₉, O and S; Y² is selected from C—R₆ and N; and Y³ is C—R₅; or (b) Y³ is selected from N—R₁₉, O and S; Y² is selected from C—R₆ and N; and Y is C—R₇; and for (A3) when p is 0, Y is selected from N—R₁₉, O and S; and Y² is selected from C—R₆ and N;
R₄, R₅, R₆ and R₇ are each independently selected from H and R₂₀, or one of R₄ and R₅, or R₅ and R₆, or R₆ and R₇ together with the carbon atoms to which they are attached form a 6-membered aryl, or a 5- or 6-membered cyclic, heterocyclic, or heteroaryl ring optionally substituted with up to three independently selected optional R₂₀ groups;
R₈ is selected from selected from H, nitrogen protecting groups and R₂₀;
X₃ is selected from C═O, C—OH and C—R′″; or Y⁵ is selected from C═O, C—OH, C—NH₂ and C—R′″; with the carbon forming part of the ring; and
when X₃ or Y⁵ is C═O then [symbol] represents an α,β-unsaturated double bond conjugated with the C═O; and when X₃ is C—OH or C—R′″ or Y⁵ is C—OH, C—NH₂ or C—R′″ then [symbol] represents the double bonds of an aromatic 6-membered ring and R₃ is absent;
wherein R′″ is a prodrug moiety containing carbonyl, carbamoyl, glycosyl, O-amino, O-acylamino, para-aminobenzyl ether, peptidyl or phosphate groups;
X₁ is selected from O, S, NR₂₁, CR₂₁R₂₂, CR₂₁R₂₂O, C(═O), C(═O)NR₂₁, NR₂₁C(═O), C(O)—R^{A}—C(O)—NH, C(O)—R^{A}—NH—C(O), C(O)—NH—R^{A}—C(O), NH—C(O)—R^{A}—C(O), NH—C(O)—R^{A}—C(O)—NH, NH—C(O)—R^{A}—NH—C(O), C(O)—NH—R^{A}—NH—C(O), C(O)—NH—R^{A}—C(O)—NH, O—C(O) and C(O)—O or is absent;
L is selected from an amino acid, a peptide chain having from 2 to 12 amino acids, a paraformaldehyde chain —(OCH₂)₁₋₂₄—, a polyethylene glycol chain —(OCH₂CH₂)₁₋₁₂— and —(CH₂)ₘ—Y⁶—(CH₂)ₙ— wherein
m is an integer selected from 0 to 12,
n is an integer selected from 0 to 12, and
Y⁶ is selected from —(CH₂)_{z}— and a group (L1) that is selected from arylene, monocyclic heteroarylene, monocyclic cycloalkylene, monocyclic cycloalkenylene and monocyclic heterocyclylene groups optionally substituted with up to three independently selected optional R₂₀ groups;
z is an integer selected from 1 to 5;
X₂ is selected from O, S, NR₂₃, CR₂₃R₂₄, CR₂₃R₂₄O, C(═O), C(═O)NR₂₃, NR₂₄C(═O), C(O)—R^{A}—C(O)—NH, C(O)—R^{A}—NH—C(O), C(O)—NH—R^{A}—C(O), NH—C(O)—R^{A}—C(O), NH—C(O)—R^{A}—C(O)—NH, NH—C(O)—R^{A}—NH—C(O), C(O)—NH—R^{A}—NH—C(O), C(O)—NH—R^{A}—C(O)—NH, O—C(O) and C(O)—O or is absent;
B is a polycyclic group selected from: 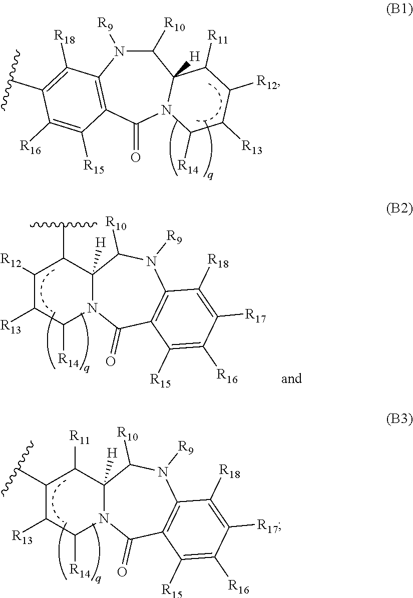
the dotted lines indicate the optional presence of one or more double bonds;
q is 0 or 1;
and R₉ and R₁₀ are selected such that either:
(i) R₉ and R₁₀ together form a double bond;
(ii) R₉ is H and R₁₀ is OH;
(iii) R₉ is H and R₁₀ is OC₁₋₆ alkyl;
(iv) R₉ is selected from SO₃H, nitrogen protecting groups and R₂₀; and
R₁₀ is H; or
(v) R₉ is H or C₁₋₆ alkyl, and R₁₀ is oxo or H;
R₁₁, R₁₂, R₁₃ and R₁₄ are independently selected from H, R₂₀, R₂₅, ═CH₂, ═CH—(CH₂)ₛ—CH₃, ═CH—(CH₂)ₛ—R₂₅, ═O, (CH₂)ₛ—OR₂₅, (CH₂)ₛ—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)ₛ—SO₂R₂₅, O—SO₂R₂₅, (CH₂)ₛ—C(O)R₂₅ and (CH₂)ₛ—C(O)NR₂₅R₂₆;
or one of R₁₁ and R₁₂, R₁₂ and R₁₃, or R₁₃ and R₁₄ together with the carbon atoms to which they are attached form a 6-membered aryl, or a 5- or 6-membered cyclic, heterocyclic, or heteroaryl ring optionally substituted with up to three independently selected optional R₂₀ groups;
each s is an integer independently selected from 0 to 6;
each t is an integer independently selected from 1 to 6;
R₁₅, R₁₆, R₁₇ and R₁₈ are independently selected from H and R₂₀;
each R₂₀ is independently selected from (CH₂)ⱼ—OH, C₁₋₆ alkyl, OC₁₋₆ alkyl, OCH₂Ph, (CH₂)ⱼ—CO₂R₂₇, O—(CH₂)ₖ—NR₂₇R₂₈, (CH₂)ⱼ—NR₂₇R₂₈, C(═O)—NH—(CH₂)ₖ—NR₂₇R₂₈, C(═O)—NH—C₆H₄—(CH₂)ⱼ—R₂₇ and C(═O)—NH—(CH₂)ₖ—C(═NH)NR₂₇R₂₈;
each j is an integer independently selected from 0 to 6;
each k is an integer independently selected from 1 to 6;
each R₁₉, R₂₁, R₂₂, R₂₃, R₂₄, R₂₆, R₂₇ and R₂₈ is independently selected from H and C₁₋₆ alkyl; and
each R₂₅ is independently selected from H, C₁₋₁₂ alkyl, C₅₋₉ heteroaryl, C₆₋₁₅ heteroarylalkyl, phenyl and C₇₋₁₂ aralkyl groups; wherein the heteroaryl, heteroarylalkyl, phenyl and aralkyl groups are optionally substituted with up to three independently selected optional R₂₀ groups;
each R^{A} is independently selected from:
—NR^{B}-T¹—NR^{C}— where R^{B} and R^{c} are each independently selected from H and C₁₋₈ alkyl, or together R^{B} and R^{c} join to form a ring and together are (CH₂)₂₋₃, where T¹ is selected from —C(O), —C(O)(CH₂)₀₋₅₀C(O)—, —C(O)PhC(O)— where Ph is 1,3- or 1,4-phenylene;
-het- wherein het is a mono-, bi-, or tricyclic heteroarylene of 5 to 12 members, containing one, two, or three heteroatoms independently selected from O, N, S, P and B, wherein het is optionally substituted up to three independently selected optional R₂₀ groups;
—X^{A}—T²—X^{A}—, where T² is: 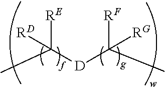
wherein each X^{A} is independently selected from a bond, —NH—, —N(C₁₋₈ alkyl)-, —O— and —S—,
each R^{D}, R^{E}, R^{F}, and R^{G} are each independently H or R₂₀, or R^{D} and R^{E} form a ring system, or R^{F} and R^{G} form a ring system, or both R^{D} and R^{E}, and R^{F} and R^{G} independently form ring systems, where said ring systems are independently selected from —C₁-C₁₀ heterocyclyl or —C₃-C₈ carbocyclycl, or R^{D}, R^{E}, R^{F}, and R^{G} are each bonds to different carbons on D, wherein f and g are each independently an integer from 0 to 50 and w is an integer from 1 to 50, and wherein D is a bond or is selected from the group consisting of —S—, —C₁-C₈ alkylene-, —C₆-C₁₄ arylene-, —C₆-C₁₄ heteroarylene-, —C₁-C₈ heteroalkylene-, —C₇-C₂₂ aralkylene, —C₁-C₁₀ heterocyclo and —C₃-C₈ carbocyclo, where said —C₁-C₈ alkylene-, —C₆-C₁₄ arylene-, —C₆-C₁₄ heteroarylene-, —C₁-C₈ heteroalkylene-, —C₇-C₂₂ aralkylene, —C₁-C₁₀ heterocyclo and —C₃-C₈ carbocyclo are optionally substituted up to three independently selected optional R₂₀ groups;
with the proviso that when the compound is: 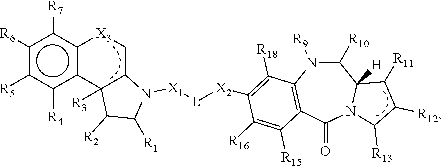
that at least one of R₁₁, R₁₂ and R₁₃ is independently selected from C₅₋₉ heteroaryl, C₆₋₁₅ heteroarylalkyl, phenyl and C₇₋₁₂ aralkyl groups and these groups are optionally substituted with up to three independently selected optional R₂₀ groups, or that one of R₁₁ and R₁₂ or R₁₂ and R₁₃, or R₁₃ together with the carbon atoms to which they are attached form a 6-membered aryl, or a 5- or 6-membered cyclic, heterocyclic, or heteroaryl ring optionally substituted with up to three independently selected optional R₂₀ groups;
with the proviso that R₅ and R₆ are each independently selected from H and R₂₀ when B, q and A are selected as (B1), 0 and (A4) respectively;
with the proviso that when R₂ is C₁₋₆ alkyl or H, that R₉ and R₁₀ are selected from options (i), (ii), (iii) or (iv); and
with the proviso that when (v) R₉ is H or C₁₋₆ alkyl, and R₁₁ is oxo or H; then either R₂ is —CH₂-halogen and R₃ is H;
or R₂ and R₃ together with the carbon atoms to which they are attached form a cyclopropyl ring.

2. A method of treatment according to claim 1, wherein one of R₁₈ and R₁₂, R₁₂ and R₁₃, or R₁₃ and R₁₄ together with the carbon atoms to which they are attached form a 6-membered aryl, or a 5- or 6-membered cyclic, heterocyclic, or heteroaryl ring optionally substituted with up to three independently selected optional R₂₀ groups.

3. A compound of formula (I):
A-X₁-L-X₂-B (I)
and salts, solvates and tautomers thereof,
wherein;
A is a group selected from: 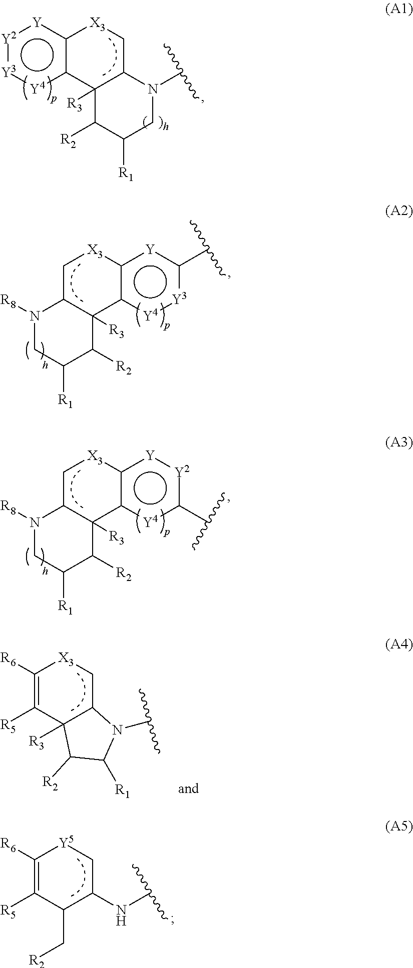
h is 0 or 1;
R, is selected from H and halogen;
either R₂ is selected from —CH₂-halogen, C₁₋₆ alkyl and H, and R₃ is H; or R₂ and R₃ together with the carbon atoms to which they are attached form a cyclopropyl ring;
p is 0 or 1; and when p is 1 then Y is C—R₇, Y² is C—R₆, Y³ is C—R₅ and Y⁴ is C—R₄; and for (A1) and (A2) when p is 0 either (a) Y is selected from N—R₁₉, O and S; Y² is selected from C—R₆ and N; and Y³ is C—R₅; or (b) Y³ is selected from N—R₁₉, O and S; Y² is selected from C—R₆ and N; and Y is C—R₇; and for (A3) when p is 0, Y is selected from N—R₁₉, O and S; and Y² is selected from C—R₆ and N;
R₄, R₅, R₆ and R₇ are each independently selected from H and R₂₀, or one of R₄ and R₅, or R₅ and R₆, or R₆ and R₇ together with the carbon atoms to which they are attached form a 6-membered aryl, or a 5- or 6-membered cyclic, heterocyclic, or heteroaryl ring optionally substituted with up to three independently selected optional R₂₀ groups;
R₈ is selected from selected from H, nitrogen protecting groups and R₂₀;
X₃ is selected from C═O, C—OH and C—R′″; or Y⁵ is selected from C═O, C—OH, C—NH₂ and C—R′″; with the carbon forming part of the ring; and
when X₃ or Y⁵ is C═O then [symbol] represents an α,β-unsaturated double bond conjugated with the C═O; and when X₃ is C—OH or C—R′″; or Y⁵ is C—OH, C—NH₂ or C—R′″ then [symbol] represents the double bonds of an aromatic 6-membered ring and R₃ is absent;
wherein R′″ is a prodrug moiety containing carbonyl, carbamoyl, glycosyl, O-amino, O-acylamino, para-aminobenzyl ether, peptidyl or phosphate groups
X₁ is selected from O, S, NR₂₁, CR₂₁R₂₂, CR₂₁R₂₂O, C(═O), C(═O)NR₂₁, NR₂₁C(═O), C(O)—R^{A}—C(O)—NH, C(O)—R^{A}—NH—C(O), C(O)—NH—R^{A}—C(O), NH—C(O)—R^{A}—C(O), NH—C(O)—R^{A}—C(O)—NH, NH—C(O)—R^{A}—NH—C(O), C(O)—NH—R^{A}—NH—C(O), C(O)—NH—R^{A}—C(O)—NH, O—C(O) and C(O)—O or is absent;
L is selected from an amino acid, a peptide chain having from 2 to 12 amino acids, a paraformaldehyde chain —(OCH₂)₁₋₂₄—, a polyethylene glycol chain —(OCH₂CH₂)₁₋₁₂— and —(CH₂)ₘ—Y⁶—(CH₂)ₙ— wherein
m is an integer selected from 0 to 12,
n is an integer selected from 0 to 12, and
Y⁶ is selected from —(CH₂)_{z}— and a group (L1) that is selected from arylene, monocyclic heteroarylene, monocyclic cycloalkylene, monocyclic cycloalkenylene and monocyclic heterocyclylene groups optionally substituted with up to three independently selected optional R₂₀ groups;
z is an integer selected from 1 to 5;
X₂ is selected from O, S, NR₂₃, CR₂₃R₂₄, CR₂₃R₂₄O, C(═O), C(═O)NR₂₃, NR₂₄C(═O), C(O)—R^{A}—C(O)—NH, C(O)—R^{A}—NH—C(O), C(O)—NH—R^{A}—C(O), NH—C(O)—R^{A}—C(O), NH—C(O)—R^{A}—C(O)—NH, NH—C(O)—R^{A}—NH—C(O), C(O)—NH—R^{A}—NH—C(O), C(O)—NH—R^{A}—C(O)—NH, O—C(O) and C(O)—O or is absent;
B is a polycyclic group selected from: 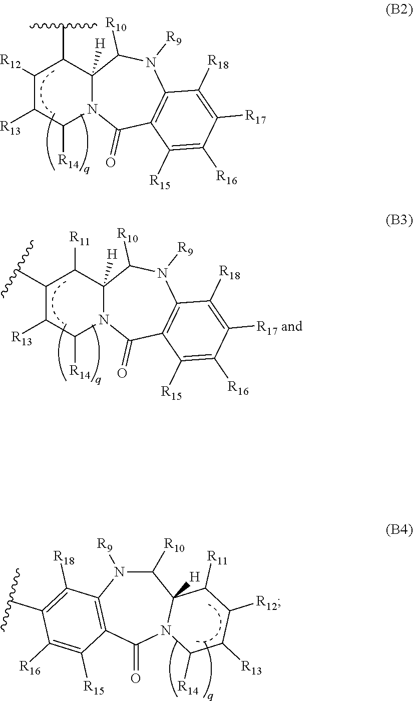
the dotted lines indicate the optional presence of one or more double bonds;
q is 0 or 1;
and R₉ and R₁₀ are selected such that either:
(i) R₉ and R₁₀ together form a double bond;
(ii) R₉ is H and R₁₀ is OH;
(iii) R₉ is H and R₁₀ is OC₁₋₆ alkyl;
(iv) R₉ is selected from SO₃H, nitrogen protecting groups and R₂₀; and
R₁₀ is H; or
(v) R₉ is H or C₁₋₆ alkyl, and R₁₀ is oxo or H R₁₁, R₁₂, R₁₃ and R₁₄ are independently selected from H, R₂₀, R₂₅, ═CH₂, ═CH—(CH₂)ₛ—CH₃, ═CH—(CH₂)ₛ—R₂₅, ═O, (CH₂)ₛ—OR₂₅, (CH₂)ₛ—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)ₛ—SO₂R₂₅, O—SO₂R₂₅, (CH₂)ₛ—C(O)R₂₅ and (CH₂)ₛ—C(O)NR₂₅R₂₆;
or one of R₁₁ and R₁₂, R₁₂ and R₁₃, or R₁₃ and R₁₄ together with the carbon atoms to which they are attached form a 6-membered aryl, or a 5- or 6-membered cyclic, heterocyclic, or heteroaryl ring optionally substituted with up to three independently selected optional R₂₀ groups;
each s is an integer independently selected from 0 to 6;
each t is an integer independently selected from 1 to 6;
R₁₅, R₁₆, R₁₇ and R₁₈ are independently selected from H and R₂₀;
each R₂₀ is independently selected from (CH₂)ⱼ—OH, C₁₋₆ alkyl, OC₁₋₆ alkyl, OCH₂Ph, (CH₂)ⱼ—CO₂R₂₇, O—(CH₂)ₖ—NR₂₇R₂₈, (CH₂)ⱼ—NR₂₇R₂₈, C(═O)—NH—(CH₂)ₖ—NR₂₇R₂₈; C(═O)—NH—C₆H₄—(CH₂)ⱼ—R₂₇ and C(═O)—NH—(CH₂)ₖ—C(═NH)NR₂₇R₂₈;
each j is an integer independently selected from 0 to 6;
each k is an integer independently selected from 1 to 6;
each R₁₉, R₂₁, R₂₂, R₂₃, R₂₄, R₂₆, R₂₇ and R₂₈ is independently selected from H and C₁₋₆ alkyl; and
each R₂₅ is independently selected from H, C₁₋₂ alkyl, C₅₋₉ heteroaryl, C₆₋₁₅ heteroarylalkyl, phenyl and C₇₋₁₂ aralkyl groups; wherein the heteroaryl, heteroarylalkyl, phenyl and aralkyl groups are optionally substituted with up to three independently selected optional R₂₀ groups;
each R^{A} is independently selected from:
—NR^{B}—T¹—NR^{C}— where R^{B} and R^{c} are each independently selected from H or C₁₋₈ alkyl, or together R^{B} and R^{c} join to form a ring and together are (CH₂)₂₋₃, where T¹ is selected from —C(O), —C(O)(CH₂)₀₋₅₀C(O)—, —C(O)PhC(O)— where Ph is 1,3- or 1,4-phenylene;
-het- wherein het is a mono-, bi-, or tricyclic heteroarylene of 5 to 12 members, containing one, two, or three heteroatoms independently selected from O, N, S, P and B, wherein het is optionally substituted up to three independently selected optional R₂₀ groups;
—X^{A}—T²—X^{A}—, where T² is: 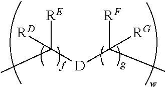
wherein each X^{A} is independently selected from a bond, —NH—, —N(C₁₋₈ alkyl)-, —O— and —S—,
each R^{D}, R^{E}, R^{F}, and R^{G} are each independently H or R₂₀, or R^{D} and R^{E} form a ring system, or R^{F} and R^{G} form a ring system, or both R^{D} and R^{E}, and R^{F} and R^{G} independently form ring systems, where said ring systems are independently selected from —C₁-C₁₀ heterocyclyl or —C₃-C₈ carbocyclycl, or R_{D}, R^{E}, R_{F}, and R^{G} are each bonds to different carbons on D, wherein f and g are each independently an integer from 0 to 50 and w is an integer from 1 to 50, and wherein D is a bond or is selected from the group consisting of —S—, —C₁-C₈ alkylene-, —C₆-C₁₄ arylene-, —C₆-C₁₄ heteroarylene-, —C₁-C₈ heteroalkylene-, —C₇-C₂₂ aralkylene, —C₁-C₁₀ heterocyclo and —C₃-C₈ carbocyclo, where said —C₁-C₈ alkylene-, —C₆-C₁₄ arylene-, —C₆-C₁₄ heteroarylene-, —C₁-C₈ heteroalkylene-, —C₇-C₂₂ aralkylene, —C₁-C₁₀ heterocyclo and —C₃-C₈ carbocyclo are optionally substituted up to three independently selected optional R₂₀ groups;
with the proviso that when R₂ is C₁₋₆ alkyl or H, that R₉ and R₁₀ are selected from options (i), (ii), (iii) or (iv); and
with the proviso that when (v) R₉ is H or C₁₋₆ alkyl, and R₁₀ is oxo or H; then either R₂ is —CH₂-halogen and R₃ is H;
or R₂ and R₃ together with the carbon atoms to which they are attached form a cyclopropyl ring.

4. A compound of formula (I) and salts, solvates and tautomers thereof according to claim 3, wherein A is (A1).

5. A compound of formula (I) according to claim 3, wherein the compound has the formula (III): 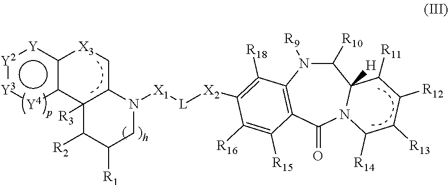
and salts, solvates and tautomers thereof.

6. A compound of formula (I) according to claim 3, wherein the compound has the formula (VI): 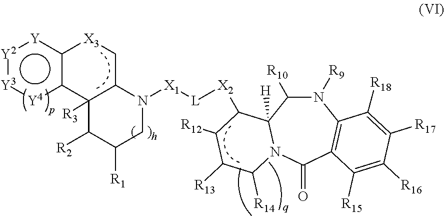
and salts, solvates and tautomers thereof.

7. A compound of formula (I) according to claim 3, wherein the compound has the formula (IX): 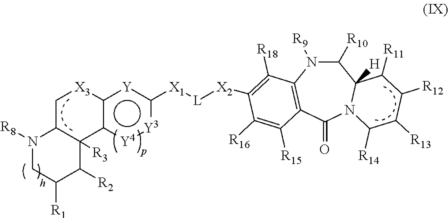
and salts, solvates and tautomers thereof.

8. A compound of formula (I) according to claim 3, wherein the compound has the formula (XII): 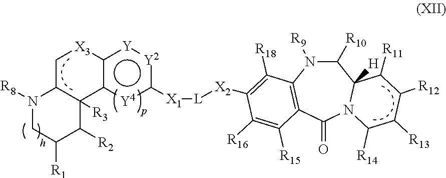
and salts, solvates and tautomers thereof.

9. A compound of formula (I) according to claim 3, wherein the compound has the formula (XIV): 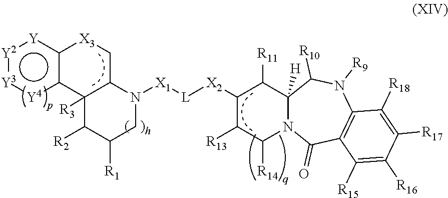
and salts, solvates and tautomers thereof.

10. A compound of formula (I) and salts, solvates and tautomers thereof according to claim 3, wherein one of R₁₁ and R₁₂, R₁₂ and R₁₃, or R₁₃ and R₁₄ together with the carbon atoms to which they are attached form a 6-membered aryl, or a 5- or 6-membered cyclic, heterocyclic, or heteroaryl ring optionally substituted with up to three independently selected optional R₂₀ groups.

11. A compound of formula (I) and salts, solvates and tautomers thereof according to claim 3, wherein X₁ is selected from C(═O) and NHC(═O).

12. A compound of formula (I) and salts, solvates and tautomers thereof according to claim 3, wherein X₂ is selected from O and CH₂, or is absent.

13. A compound of formula (I) and salts, solvates and tautomers thereof according to claim 3, wherein L is selected from —(CH₂)ₘ—(CH₂)_{z}—(CH₂)ₙ—, 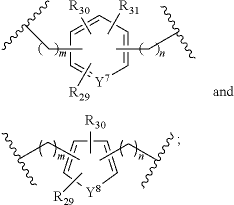
and
R₂₉, R₃₀ and R₃₁ are each independently selected from H and R₂₀.

14. A compound of formula (I) and salts, solvates and tautomers thereof according to claim 3, wherein L is —(CH₂)₃—.

15. A pharmaceutical composition comprising a compound of formula (I) and salts, solvates and tautomers thereof of claim 3 and a pharmaceutically acceptable carrier or diluent.

16. A method of treatment of a patient suffering from a proliferative disease, comprising administering to said patient a therapeutically effective amount of a compound of formula (I) and salts, solvates and tautomers thereof of claim 3.

17. A method of treatment according to claim 16, wherein the proliferative disease is selected from bladder cancer, bone cancer, bowel cancer, brain cancer, breast cancer, cervical cancer, colon cancer, head and neck cancer, leukemia, liver cancer, lung cancer, lymphoma, melanoma, oesophageal cancer, oral cancer, ovarian cancer, pancreatic cancer, prostate cancer, rectal cancer, renal cancer, retinoblastoma, sarcoma, skin cancer, stomach cancer, testicular cancer, thyroid cancer and uterine cancer.
